(19) United States
(12) Patent Application Publication
(10) Pub. No.: US 2020/0239896 A1
(43) Pub. Date: Jul. 30, 2020

(54) GENETIC KNOCKOUTS IN WOOD-LJUNGDAHL MICROORGANISMS

(71) Applicant: LanzaTech, Inc., Skokie, IL (US)

(72) Inventor: James Daniell, Auckland (NZ)

(21) Appl. No.: 16/650,437

(22) PCT Filed: Sep. 28, 2018
(86) PCT No.: PCT/US2018/053587

§ 371 Date: Mar. 25, 2020

Related U.S. Application Data
(60) Provisional application No. 62/565,000, filed on Sep. 28, 2017.

(51) Int. Cl. C12N 15/74; C12P 7/00

(52) U.S. Cl. CPC C12N 15/74; C12Y 203/01009; C12P 7/00

(57) ABSTRACT

The invention provides genetically engineered Wood-Ljungdahl microorganisms comprising one or more disrupted genes to strategically divert carbon flux away from nonessential or undesirable products and towards products of interest. The expression strategies of the invention enable the production of useful fuels and chemicals from gaseous substrates, such as carbon monoxide, carbon dioxide, and/or hydrogen.

### CROSS REFERENCE TO RELATED APPLICATIONS

This application is the U.S. national phase of International Patent Application No. PCT/US2018/053587 filed on Sep. 28, 2018, which claims the benefit of U.S. Provisional Patent Application No. 62/565,000 filed Sep. 28, 2017, both of which are incorporated by reference in their entirety.

### BACKGROUND OF THE INVENTION

It has long been recognized that catalytic processes, such as the Fischer-Tropsch process, may be used to convert gases containing carbon dioxide (CO₂), carbon monoxide (CO), and/or hydrogen (H₂), such as industrial waste gas or syngas, into a variety of fuels and chemicals. Recently, however, gas fermentation has emerged as an alternative platform for the biological fixation of such gases. In particular, C1-fixing microorganisms have been demonstrated to convert gases containing CO₂, CO, and/or H₂ into products such as ethanol and 2,3-butanediol. Efficient production of such products may be limited, however, by slow microbial growth, limited gas uptake, sensitivity to toxins, or diversion of carbon substrates into undesired byproducts. Accordingly, there remains a need for genetically engineered microorganisms having improved characteristics.

### DESCRIPTION OF THE FIGURES

FIG. 1 is a diagram showing key production pathways and key metabolic nodes (indicated with boxes) in Wood-Ljungdahl microorganisms. Improving carbon flux through these nodes, e.g. by disrupting expression of certain genes, improves production of downstream products.

### DESCRIPTION OF THE INVENTION

The invention provides non-naturally occurring microorganisms comprising at least one disrupted gene. In the microorganisms of the invention, carbon flux is strategically diverted away from nonessential or undesirable products and towards products of interest. In certain embodiments, these disrupted genes divert carbon flux away from nonessential or undesirable metabolic nodes and through target metabolic nodes to improve production of products downstream of those target metabolic nodes.

The microorganisms of the invention are derived from parental bacteria such as Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, or Thermoanaerobacter kiuvi. In a preferred embodiment, the parental bacterium is Clostridium autoethanogenum, Clostridium ljungdahlii, or Clostridium ragsdalei. In a particularly preferred embodiment, the parental bacterium is Clostridium autoethanogenum.

In one embodiment, the invention provides a non-naturally occurring Wood-Ljungdahl bacterium comprising a heterologous thiolase and a disruptive mutation in one or more genes encoding, for example, one or more of NAD-dependent electron-bifurcating [FeFe]-hydrogenase, glutamate synthase, citramalate synthase, acetolactate decarboxylase, lactate dehydrogenase, acetate kinase, phosphate transacetylase, and aldehyde dehydrogenase, wherein the non-naturally occurring bacterium has improved carbon flux through acetoacetyl-CoA compared to a parental bacterium. Specifically, the expression of the one or more genes is decreased or eliminated compared to the parental bacterium.

In such an embodiment, the the non-naturally occurring bacterium may produce a product such as acetone, isopropanol, 3-hydroxyisovaleryl-CoA, 3-hydroxyisovalerate, isobutylene, isopentenyl pyrophosphate, dimethylallyl pyrophosphate, isoprene, farnesene, 3-hydroxybutyryl-CoA, crotonyl-CoA, 3-hydroxybutyrate, 3-hydroxybutyrylaldehyde, 1,3-butanediol, 2-hydroxyisobutyryl-CoA, 2-hydroxyisobutyrate, butyryl-CoA, butyrate, butanol, caproate, hexanol, octanoate, octanol, 1,3-hexanediol, 2-buten-1-ol, isovaleryl-CoA, isovalerate, or isoamyl alcohol.

For example, when the parental bacterium is Clostridium autoethanogenum, (a) the NAD-dependent electron-bifurcating [FeFe]-hydrogenase may be selected from the group consisting of CAETHG_1576, CAETHG_1578, CAETHG_3569, CAETHG_3570, and CAETHG_3571, (b) the glutamate synthase may be selected from the group consisting of CAETHG_0477, CAETHG_1580, CAETHG_3850, and CAETHG_3851, (c) the citramalate synthase may be CAETHG_2751, (d) the acetolactate decarboxylase may be CAETHG_2932, (e) the lactate dehydrogenase may be CAETHG_1147, (f) the acetate kinase may be CAETHG_3359, (g) the phosphate transacetylase may be CAETHG_3358, or (h) the aldehyde dehydrogenase may be selected from the group consisting of CAETHG_1819, CAETHG_3287, and CAETHG_1830.

In another embodiment, the invention provides a non-naturally occurring Wood-Ljungdahl bacterium comprising a disruptive mutation in one or more genes, wherein the non-naturally occurring bacterium has improved carbon flux through chorismate compared to a parental bacterium.

The one or more genes encode, for example, one or more of purine-nucleoside phosphorylase, lactate permease, cystathionine gamma-lyase, adenine phosphoribosyltransferase, 5′-nucleotidase/3′-nucleotidase/exopolyphosphatase, small conductance mechanosensitive channel, arginine deiminase, LL-diaminopimelate aminotransferase apoenzyme, and phosphopentomutase. Specifically, the expression of the one or more genes is decreased or eliminated compared to the parental bacterium.

In such an embodiment, the the non-naturally occurring bacterium may produce a product such as chorismate, para-hydroxybenzoic acid, salicylate, 2-aminobenzoate, dihydroxybenzoate, 4-hydroxycyclohexane carboxylic acid, and salts and ions thereof.

For example, when the parental bacterium is Clostridium autoethanogenum, the one or more genes may encode one or more of CAETHG_0160, CAETHG_0248, CAETHG_0498, CAETHG_1270, CAETHG_1371, CAETHG_2107, CAETHG_3021, CAETHG_3510, and CAETHG_3924; when the parental bacterium is Clostridium ljungdahlii, the one or more genes may encode one or more of CLJU_c20750, CLJU_c21610, CLJU_c24380, CLJU_c33720, CLJU_c34740, CLJU_c42810, CLJU_c09270, CLJU_c14280, and CLJU_c18150; and when the parental bacterium is Clostridium ragsdalei and the one or more genes may encode one or more of CLRAG_19250, CLRAG_31200, CLRAG_25120, CLRAG_24560, CLRAG_14800, CLRAG_25620, CLRAG_09600, or CLRAG_00520.

The invention also provides methods of producting products by culturing the microorganism of the invention in the presence of a substrate, such as a gaseous substrate comprising one or more of CO, CO₂, and/or H₂.

The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refers to a microorganism containing such a genetic modification, genetic alteration, or genetic engineering. These terms may be used to differentiate a lab-generated microorganism from a naturally-occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization.

“Recombinant” indicates that a nucleic acid, protein, or microorganism is the product of genetic modification, engineering, or recombination. Generally, the term “recombinant” refers to a nucleic acid, protein, or microorganism that contains or is encoded by genetic material derived from multiple sources, such as two or more different strains or species of microorganisms.

“Wild type” refers to the typical form of an organism, strain, gene, or characteristic as it occurs in nature, as distinguished from mutant or variant forms.

“Endogenous” refers to a nucleic acid or protein that is present or expressed in the wild-type or parental microorganism from which the microorganism of the invention is derived. For example, an endogenous gene is a gene that is natively present in the wild-type or parental microorganism from which the microorganism of the invention is derived. In one embodiment, the expression of an endogenous gene may be controlled by an exogenous regulatory element, such as an exogenous promoter.

“Exogenous” refers to a nucleic acid or protein that originates outside the microorganism of the invention. For example, an exogenous gene or enzyme may be artificially or recombinantly created and introduced to or expressed in the microorganism of the invention. An exogenous gene or enzyme may also be isolated from a heterologous microorganism and introduced to or expressed in the microorganism of the invention. Exogenous nucleic acids may be adapted to integrate into the genome of the microorganism of the invention or to remain in an extra-chromosomal state in the microorganism of the invention, for example, in a plasmid. “Heterologous” refers to a nucleic acid or protein that is derived from a different strain or species and introduced to or expressed in the microorganism of the invention.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides or nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene products.”

The terms “polypeptide”, “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

“Enzyme activity,” or simply “activity,” refers broadly to enzymatic activity, including, but not limited, to the activity of an enzyme, the amount of an enzyme, or the availability of an enzyme to catalyze a reaction. Accordingly, “increasing” enzyme activity includes increasing the activity of an enzyme, increasing the amount of an enzyme, or increasing the availability of an enzyme to catalyze a reaction. Similarly, “decreasing” enzyme activity includes decreasing the activity of an enzyme, decreasing the amount of an enzyme, or decreasing the availability of an enzyme to catalyze a reaction.

“Mutated” refers to a nucleic acid or protein that has been modified in the microorganism of the invention compared to the wild-type or parental microorganism from which the microorganism of the invention is derived. In one embodiment, the mutation may be a deletion, insertion, or substitution in a gene encoding an enzyme. In another embodiment, the mutation may be a deletion, insertion, or substitution of one or more amino acids in an enzyme.

“Disrupted gene” refers to a gene that has been modified in some way to reduce or eliminate expression of the gene, regulatory activity of the gene, or activity of an encoded protein or enzyme. The disruption may partially inactivate, fully inactivate, or delete the gene or enzyme. The disruption may be a knockout (KO) mutation that fully eliminates the expression or activity of a gene, protein, or enzyme. The disruption may also be a knock-down that reduces, but does not entirely eliminate, the expression or activity of a gene, protein, or enzyme. The disruption may be be anything that reduces, prevents, or blocks the biosynthesis of a product produced by an enzyme. The disruption may include, for example, a mutation in a gene encoding a protein or enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, RNAi, TALEN, siRNA, CRISPR, or CRISPRi) or protein which inhibits the expression of a protein or enzyme. The disruption may be introduced using any method known in the art. For the purposes of the present invention, disruptions are laboratory-generated, not naturally occurring.

“Codon optimization” refers to the mutation of a nucleic acid, such as a gene, for optimized or improved translation of the nucleic acid in a particular strain or species. Codon optimization may result in faster translation rates or higher translation accuracy. In a preferred embodiment, the genes of the invention are codon optimized for expression in Clostridium, particularly Clostridium autoethanogenum, Clostridium ljungdahlii, or Clostridium ragsdalei. In a further preferred embodiment, the genes of the invention are codon optimized for expression in Clostridium autoethanogenum LZ1561, which is deposited under DSMZ accession number DSM23693.

“Overexpressed” refers to an increase in expression of a nucleic acid or protein in the microorganism of the invention compared to the wild-type or parental microorganism from which the microorganism of the invention is derived. Overexpression may be achieved by any means known in the art, including modifying gene copy number, gene transcription rate, gene translation rate, or enzyme degradation rate.

The term “variants” includes nucleic acids and proteins whose sequence varies from the sequence of a reference nucleic acid and protein, such as a sequence of a reference nucleic acid and protein disclosed in the prior art or exemplified herein. The invention may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like. Homologous genes from other microorganisms are also examples of functionally equivalent variants. These include homologous genes in species such as Clostridium acetobutylicum, Clostridium beijerinckii, or Clostridium ljungdahlii, the details of which are publicly available on websites such as Genbank or NCBI. Functionally equivalent variants also include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

Nucleic acids may be delivered to a microorganism of the invention using any method known in the art. For example, nucleic acids may be delivered as naked nucleic acids or may be formulated with one or more agents, such as liposomes. The nucleic acids may be DNA, RNA, cDNA, or combinations thereof, as is appropriate. Restriction inhibitors may be used in certain embodiments. Additional vectors may include plasmids, viruses, bacteriophages, cosmids, and artificial chromosomes. In a preferred embodiment, nucleic acids are delivered to the microorganism of the invention using a plasmid. By way of example, transformation (including transduction or transfection) may be achieved by electroporation, ultrasonication, polyethylene glycol-mediated transformation, chemical or natural competence, protoplast transformation, prophage induction, or conjugation. In certain embodiments having active restriction enzyme systems, it may be necessary to methylate a nucleic acid before introduction of the nucleic acid into a microorganism.

Furthermore, nucleic acids may be designed to comprise a regulatory element, such as a promoter, to increase or otherwise control expression of a particular nucleic acid. The promoter may be a constitutive promoter or an inducible promoter. Ideally, the promoter is a Wood-Ljungdahl pathway promoter, a ferredoxin promoter, a pyruvate:ferredoxin oxidoreductase promoter, an Rnf complex operon promoter, an ATP synthase operon promoter, or a phosphotransacetylase/acetate kinase operon promoter.

A “microorganism” is a microscopic organism, especially a bacterium, archea, virus, or fungus. The microorganism of the invention is typically a bacterium. Herein, recitation of “microorganism” should be taken to encompass “bacterium.”

A “parental microorganism” is a microorganism used to generate a microorganism of the invention. The parental microorganism may be a naturally-occurring microorganism (i.e., a wild-type microorganism) or a microorganism that has been previously modified (i.e., a mutant or recombinant microorganism). The microorganism of the invention may be modified to express or overexpress one or more enzymes that were not expressed or overexpressed in the parental microorganism. Similarly, the microorganism of the invention may be modified to contain one or more genes that were not contained by the parental microorganism. The microorganism of the invention may also be modified to not express or to express lower amounts of one or more enzymes that were expressed in the parental microorganism. In one embodiment, the parental microorganism is Clostridium autoethanogenum, Clostridium ljungdahlii, or Clostridium ragsdalei. In a preferred embodiment, the parental microorganism is Clostridium autoethanogenum LZ1561, which was deposited on Jun. 7, 2010 with Deutsche Sammlung von Mikroorganismen und Zellkulturen GmbH (DSMZ) located at InhoffenstraB 7B, D-38124 Braunschwieg, Germany on Jun. 7, 2010 under the terms of the Budapest Treaty and accorded accession number DSM23693. This strain is described in International Patent Application No. PCT/NZ2011/000144, which published as WO 2012/015317.

The term “derived from” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes. Generally, the microorganism of the invention is derived from a parental microorganism. In one embodiment, the microorganism of the invention is derived from Clostridium autoethanogenum, Clostridium ljungdahlii, or Clostridium ragsdalei. In a preferred embodiment, the microorganism of the invention is derived from Clostridium autoethanogenum LZ1561, which is deposited under DSMZ accession number DSM23693.

The microorganism of the invention may be further classified based on functional characteristics. For example, the microorganism of the invention may be or may be derived from a C1-fixing microorganism, an anaerobe, an acetogen, an ethanologen, a carboxydotroph, and/or a methanotroph. Table 1 provides a representative list of microorganisms and identifies their functional characteristics.

TABLE 1

| | Wood-Ljungdahl | C1-fixing | Anaerobe | Acetogen | Ethanologen | Autotroph | Carboxydotroph |
|---|---|---|---|---|---|---|---|
| Acetobacterium woodii | + | + | + | + | +/−¹ | − | − |
| Alkalibaculum bacchii | + | + | + | + | + | + | + |
| Blautia producta | + | + | + | + | − | + | + |
| Butyribacterium methylotrophicum | + | + | + | + | + | + | + |
| Clostridium aceticum | + | + | + | + | − | + | + |
| Clostridium autoethanogenum | + | + | + | + | + | + | + |
| Clostridium carboxidivorans | + | + | + | + | + | + | + |
| Clostridium coskatii | + | + | + | + | + | + | + |
| Clostridium drakei | + | + | + | + | − | + | + |
| Clostridium formicoaceticum | + | + | + | + | − | + | + |
| Clostridium ljungdahlii | + | + | + | + | + | + | + |
| Clostridium magnum | + | + | + | + | − | + | +/−² |
| Clostridium ragsdalei | + | + | + | + | + | + | + |
| Clostridium scatologenes | + | + | + | + | − | + | + |
| Eubacterium limosum | + | + | + | + | − | + | + |
| Moorella thermautotrophica | + | + | + | + | + | + | + |
| Moorella thermoacetica (formerly | + | + | + | + | −³ | + | + |
| Clostridium thermoaceticum) | | | | | | | |
| Oxobacter pfennigii | + | + | + | + | − | + | + |
| Sporomusa ovata | + | + | + | + | − | + | +/−⁴ |
| Sporomusa silvacetica | + | + | + | + | − | + | +/−⁵ |
| Sporomusa sphaeroides | + | + | + | + | − | + | +/−⁶ |
| Thermoanaerobacter kiuvi | + | + | + | + | − | + | − |

¹Acetobacterium woodi can produce ethanol from fructose, but not from gas.
²It has not been investigated whether Clostridium magnum can grow on CO.
³One strain of Moorella thermoacetica, Moorella sp. HUC22-1, has been reported to produce ethanol from gas.
⁴It has not been investigated whether Sporomusa ovata can grow on CO.
⁵It has not been investigated whether Sporomusa silvacetica can grow on CO.
⁶It has not been investigated whether Sporomusa sphaeroides can grow on CO.

“Wood-Ljungdahl” refers to the Wood-Ljungdahl pathway of carbon fixation as described, e.g., by Ragsdale, Biochim Biophys Acta, 1784: 1873-1898, 2008. “Wood-Ljungdahl microorganism” refers, predictably, to a microorganism containing the Wood-Ljungdahl pathway. The microorganism of the invention is a Wood-Ljungdahl microorganism, usually a Wood-Ljungdahl bacterium. Generally, the microorganism of the invention contains a native Wood-Ljungdahl pathway. Herein, a Wood-Ljungdahl pathway may be a native, unmodified Wood-Ljungdahl pathway or it may be a Wood-Ljungdahl pathway with some degree of genetic modification (e.g., overexpression, heterologous expression, knockout, etc.) so long as it still functions to convert CO, CO₂, and/or H₂ to acetyl-CoA.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the invention. For example, a C1-carbon source may comprise one or more of CO, CO₂, CH₄, CH₃OH, or CH₂O₂. Preferably, the C1-carbon source comprises one or both of CO and CO₂. A “C1-fixing microorganism” is a microorganism that has the ability to produce one or more products from a C1-carbon source. Typically, the microorganism of the invention is a C1-fixing bacterium. In a preferred embodiment, the microorganism of the invention is derived from a C1-fixing microorganism identified in Table 1.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen). Typically, the microorganism of the invention is an anaerobe. In a preferred embodiment, the microorganism of the invention is derived from an anaerobe identified in Table 1.

An “acetogen” is a microorganism that produces or is capable of producing acetate (or acetic acid) as a product of anaerobic respiration. Typically, acetogens are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). Acetogens use the acetyl-CoA pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, N.Y., 2006). All naturally occurring acetogens are C1-fixing, anaerobic, autotrophic, and non-methanotrophic. Typically, the microorganism of the invention is an acetogen. In a preferred embodiment, the microorganism of the invention is derived from an acetogen identified in Table 1.

An “ethanologen” is a microorganism that produces or is capable of producing ethanol. Typically, the microorganism of the invention is an ethanologen. In a preferred embodiment, the microorganism of the invention is derived from an ethanologen identified in Table 1.

An “autotroph” is a microorganism capable of growing in the absence of organic carbon. Instead, autotrophs use inorganic carbon sources, such as CO and/or CO₂. Typically, the microorganism of the invention is an autotroph. In a preferred embodiment, the microorganism of the invention is derived from an autotroph identified in Table 1.

A “carboxydotroph” is a microorganism capable of utilizing CO as a sole source of carbon and energy. Typically, the microorganism of the invention is a carboxydotroph. In a preferred embodiment, the microorganism of the invention is derived from a carboxydotroph identified in Table 1.

A “methanotroph” is a microorganism capable of utilizing methane as a sole source of carbon and energy. In certain embodiments, the microorganism of the invention is a methanotroph or is derived from a methanotroph. In other embodiments, the microorganism of the invention is not a methanotroph or is not derived from a methanotroph.

More broadly, the microorganism of the invention may be derived from any genus or species identified in Table 1. For example, the microorganism may be a member of a genus selected from the group consisting of Acetobacterium, Alkalibaculum, Blautia, Butyribacterium, Clostridium, Eubacterium, Moorella, Oxobacter, Sporomusa, and Thermoanaerobacter. In particular, the microorganism may be derived from a parental bacterium selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi.

In a preferred embodiment, the microorganism of the invention is derived from the cluster of Clostridia comprising the species Clostridium autoethanogenum, Clostridium ljungdahlii, and Clostridium ragsdalei. These species were first reported and characterized by Abrini, Arch Microbiol, 161: 345-351, 1994 (Clostridium autoethanogenum), Tanner, Int J System Bacteriol, 43: 232-236, 1993 (Clostridium ljungdahlii), and Huhnke, WO 2008/028055 (Clostridium ragsdalei).

These three species have many similarities. In particular, these species are all C1-fixing, anaerobic, acetogenic, ethanologenic, and carboxydotrophic members of the genus Clostridium. These species have similar genotypes and phenotypes and modes of energy conservation and fermentative metabolism. Moreover, these species are clustered in clostridial rRNA homology group I with 16S rRNA DNA that is more than 99% identical, have a DNA G+C content of about 22-30 mol %, are gram-positive, have similar morphology and size (logarithmic growing cells between 0.5-0.7×3-5 μm), are mesophilic (grow optimally at 30-37° C.), have similar pH ranges of about 4-7.5 (with an optimal pH of about 5.5-6), lack cytochromes, and conserve energy via an Rnf complex. Also, reduction of carboxylic acids into their corresponding alcohols has been shown in these species (Perez, Biotechnol Bioeng, 110:1066-1077, 2012). Importantly, these species also all show strong autotrophic growth on CO-containing gases, produce ethanol and acetate (or acetic acid) as main fermentation products, and produce small amounts of 2,3-butanediol and lactic acid under certain conditions.

However, these three species also have a number of differences. These species were isolated from different sources: Clostridium autoethanogenum from rabbit gut, Clostridium ljungdahlii from chicken yard waste, and Clostridium ragsdalei from freshwater sediment. These species differ in utilization of various sugars (e.g., rhamnose, arabinose), acids (e.g., gluconate, citrate), amino acids (e.g., arginine, histidine), and other substrates (e.g., betaine, butanol). Moreover, these species differ in auxotrophy to certain vitamins (e.g., thiamine, biotin). These species have differences in nucleic and amino acid sequences of Wood-Ljungdahl pathway genes and proteins, although the general organization and number of these genes and proteins has been found to be the same in all species (Köpke, Curr Opin Biotechnol, 22: 320-325, 2011).

Thus, in summary, many of the characteristics of Clostridium autoethanogenum, Clostridium ljungdahlii, or Clostridium ragsdalei are not specific to that species, but are rather general characteristics for this cluster of C1-fixing, anaerobic, acetogenic, ethanologenic, and carboxydotrophic members of the genus Clostridium. However, since these species are, in fact, distinct, the genetic modification or manipulation of one of these species may not have an identical effect in another of these species. For instance, differences in growth, performance, or product production may be observed.

The microorganism of the invention may also be derived from an isolate or mutant of Clostridium autoethanogenum, Clostridium ljungdahlii, or Clostridium ragsdalei. Isolates and mutants of Clostridium autoethanogenum include JAl-1 (DSM10061) (Abrini, Arch Microbiol, 161: 345-351, 1994), LBS1560 (DSM19630) (WO 2009/064200), and LZ1561 (DSM23693) (WO 2012/015317). Isolates and mutants of Clostridium ljungdahlii include ATCC 49587 (Tanner, Int J Syst Bacteriol, 43: 232-236, 1993), PETCT (DSM13528, ATCC 55383), ERI-2 (ATCC 55380) (U.S. Pat. No. 5,593,886), C-01 (ATCC 55988) (U.S. Pat. No. 6,368,819), 0-52 (ATCC 55989) (U.S. Pat. No. 6,368,819), and OTA-1 (Tirado-Acevedo, Production of bioethanol from synthesis gas using Clostridium ljungdahlii, PhD thesis, North Carolina State University, 2010). Isolates and mutants of Clostridium ragsdalei include PI 1 (ATCC BAA-622, ATCC PTA-7826) (WO 2008/028055).

“Substrate” refers to a carbon and/or energy source for the microorganism of the invention. Typically, the substrate is gaseous and comprises a C1-carbon source, for example, CO, CO₂, and/or CH₄. Preferably, the substrate comprises a C1-carbon source of CO or CO+CO₂. The substrate may further comprise other non-carbon components, such as H₂, N₂, or electrons.

The substrate generally comprises at least some amount of CO, such as about 1, 2, 5, 10, 20, 30, 40, 50, 60, 70, 80, 90, or 100 mol % CO. The substrate may comprise a range of CO, such as about 20-80, 30-70, or 40-60 mol % CO. Preferably, the substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the substrate may comprise a relatively low amount of CO, such as about 1-10 or 1-20 mol % CO. The microorganism of the invention typically converts at least a portion of the CO in the substrate to a product. In some embodiments, the substrate comprises no or substantially no (<1 mol %) CO.

The substrate may comprise some amount of H₂. For example, the substrate may comprise about 1, 2, 5, 10, 15, 20, or 30 mol % H₂. In some embodiments, the substrate may comprise a relatively high amount of H₂, such as about 60, 70, 80, or 90 mol % H₂. In further embodiments, the substrate comprises no or substantially no (<1 mol %) H₂.

The substrate may comprise some amount of CO₂. For example, the substrate may comprise about 1-80 or 1-30 mol % CO₂. In some embodiments, the substrate may comprise less than about 20, 15, 10, or 5 mol % CO₂. In another embodiment, the substrate comprises no or substantially no (<1 mol %) CO₂.

Although the substrate is typically gaseous, the substrate may also be provided in alternative forms. For example, the substrate may be dissolved in a liquid saturated with a CO-containing gas using a microbubble dispersion generator. By way of further example, the substrate may be adsorbed onto a solid support.

The substrate and/or C1-carbon source may be a waste gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the substrate and/or C1-carbon source may be captured from the industrial process before it is emitted into the atmosphere, using any convenient method.

The substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The composition of the substrate may have a significant impact on the efficiency and/or cost of the reaction. For example, the presence of oxygen (02) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

In certain embodiments, the fermentation is performed in the absence of carbohydrate substrates, such as sugar, starch, lignin, cellulose, or hemicellulose.

The microorganism of the invention may be cultured to produce one or more products. For instance, the microorganism of the invention may produce or may be engineered to produce ethanol (WO 2007/117157), acetate (WO 2007/117157), butanol (WO 2008/115080 and WO 2012/053905), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), isoprene (WO 2013/180584), fatty acids (WO 2013/191567), 2-butanol (WO 2013/185123), 1,2-propanediol (WO 2014/036152), 1-propanol (WO 2014/0369152), chorismate-derived products (WO 2016/191625), 3-hydroxybutyrate (WO 2017/066498), and 1,3-butanediol (WO 2017/0066498). In addition to one or more target products, the microorganism of the invention may also produce ethanol, acetate, and/or 2,3-butanediol. In certain embodiments, microbial biomass itself may be considered a product.

A “native product” is a product produced by a genetically unmodified microorganism. For example, ethanol, acetate, and 2,3-butanediol are native products of Clostridium autoethanogenum, Clostridium ljungdahlii, and Clostridium ragsdalei. A “non-native product” is a product that is produced by a genetically modified microorganism, but is not produced by a genetically unmodified microorganism from which the genetically modified microorganism is derived.

Herein, reference to an acid (e.g., acetic acid or 2-hydroxyisobutyric acid) should be taken to also include the corresponding salt (e.g., acetate or 2-hydroxyisobutyrate).

“Selectivity” refers to the ratio of the production of a target product to the production of all fermentation products produced by a microorganism. The microorganism of the invention may be engineered to produce products at a certain selectivity or at a minimum selectivity. In one embodiment, a target product account for at least about 5%, 10%, 15%, 20%, 30%, 50%, or 75% of all fermentation products produced by the microorganism of the invention. In one embodiment, the target product accounts for at least 10% of all fermentation products produced by the microorganism of the invention, such that the microorganism of the invention has a selectivity for the target product of at least 10%. In another embodiment, the target product accounts for at least 30% of all fermentation products produced by the microorganism of the invention, such that the microorganism of the invention has a selectivity for the target product of at least 30%.

“Increasing the efficiency,” “increased efficiency,” and the like include, but are not limited to, increasing growth rate, product production rate or volume, product volume per volume of substrate consumed, or product selectivity. Efficiency may be measured relative to the performance of parental microorganism from which the microorganism of the invention is derived.

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Preferably the aqueous culture medium is an anaerobic microbial growth medium, such as a minimal anaerobic microbial growth medium. Suitable media are well known in the art.

The culture/fermentation should desirably be carried out under appropriate conditions for production of the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, it is generally preferable to perform the culture/fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

In certain embodiments, the fermentation is performed in the absence of light or in the presence of an amount of light insufficient to meet the energetic requirements of photosynthetic microorganisms. In certain embodiments, the microorganism of the invention is a non-photosynthetic microorganism.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably returned to the bioreactor. The cell-free permeate remaining after target products have been removed is also preferably returned to the bioreactor. Additional nutrients (such as B vitamins) may be added to the cell-free permeate to replenish the medium before it is returned to the bioreactor.

The microorganism of the invention contains at least one disrupted gene. In some embodiments, the microorganism of the invention contains more than one disrupted genes, e.g., 2, 3, 4, 5, 6, 7, 8, 9, 10, 11, 12, 13, 14, 15, 16, 17, 18, 19, 20, 25, 30, 35, 40, 45, 50, 75, 100, or 200 disrupted genes. For example, the disrupted gene may be selected from Table 2. Although representative accession numbers are provided for C. autoethanogenum, C. ljungdahlii, and C. ragsdalei, a person of ordinary skill in the art would be capable of readily identifying homologs in other Wood-Ljungdahl microorganisms.

TABLE 2

| | | | Clostridium | Clostridium | Clostridium |
|---|---|---|---|---|---|
| | | | autoethanogenum | ljungdahlii | ragsdalei |
| # | Name | EC No. | Gene(s) | Gene(s) | Gene(s) |
| 1 | Isopropylmalate/homocitrate/citramalate | 2.3.1.182 | CAETHG_2751 | CLJU_c06610 | CLRAG_18420 |
| | synthases | | | | |
| 2 | [NiFe]-hydrogenase I apoprotein, large subunit | | CAETHG_0861 | CLJU_c28660 | CLRAG_34740 |
| 3 | [NiFe]-hydrogenase I apoprotein, small subunit | | CAETHG_0862 | CLJU_c28670 | CLRAG_34750 |
| 4 | ribosomal-protein-alanine N-acetyltransferase | | CAETHG_1676 | CLJU_c38200 | CLRAG_20660 |
| 5 | 1-(5-phosphoribosyl)-5-[(5- | 5.3.1.16 | CAETHG_3262 | CLJU_c11710 | CLRAG_11830 |
| | phosphoribosylamino)methylideneamino] | | | | |
| | imidazole-4-carboxamide isomerase | | | | |
| 6 | 1-acyl-sn-glycerol-3-phosphate acyltransferase | 2.3.1.51 | CAETHG_1773 | CLJU_c39280 | CLRAG_21490 |
| 7 | 1-acyl-sn-glycerol-3-phosphate acyltransferase | 2.3.1.51 | CAETHG_2750 | CLJU_c06600 | CLRAG_18410 |
| 8 | 1-deoxy-D-xylulose 5-phosphate reductoisomerase | 1.1.1.267 | CAETHG_3391 | CLJU_c13080 | CLRAG_10710 |
| 9 | 1-deoxy-D-xylulose-5-phosphate synthase | 2.2.1.7, | CAETHG_3205 | CLJU_c11160 | CLRAG_12300 |
| | | 2.2.1.1 | | | |
| 10 | 1,2-diacylglycerol 3-alpha-glucosyltransferase | | CAETHG_0046 | CLJU_c19690 | CLRAG_39450 |
| 11 | Alcohol dehydrogenase, class IV | 1.1.1.1, | CAETHG_1078 | CLJU_c30740 | CLRAG_16180 |
| | | 1.1.1.72, | | | |
| | | 1.1.1.21, | | | |
| | | 1.1.1.2 | | | |
| 12 | alcohol dehydrogenase | 1.1.1.1, | CAETHG_1500 | CLJU_c35930 | CLRAG_06430 |
| | | 1.1.1.72, | | | |
| | | 1.1.1.21, | | | |
| | | 1.1.1.2 | | | |
| 13 | Alcohol dehydrogenase, class IV | 1.1.1.1, | CAETHG_3604 | CLJU_c15000 | CLRAG_24350 |
| | | 1.1.1.72, | | | |
| | | 1.1.1.21, | | | |
| | | 1.1.1.2 | | | |
| 14 | chaperonin GroES | | CAETHG_1573 | CLJU_c37200 | CLRAG_36640 |
| 15 | 16S rRNA (guanine527-N7)-methyltransferase | | CAETHG_2116 | CLJU_c42900 | CLRAG_25710 |
| 16 | HSP20 family protein | | CAETHG_2094, | CLJU_c42700, | CLRAG_25500 |
| | | | CAETHG_2095 | CLJU_c42690 | |
| 17 | 2-C-methyl-D-erythritol 2,4-cyclodiphosphate | 4.6.1.12 | CAETHG_2263 | CLJU_c01570 | CLRAG_27230 |
| | synthase | | | | |
| 18 | 2-C-methyl-D-erythritol 4-phosphate | 2.7.7.60 | CAETHG_1969 | CLJU_c41280 | CLRAG_23470 |
| | cytidylyltransferase | | | | |
| 19 | 2-isopropylmalate synthase | 2.3.3.13, | CAETHG_2999 | CLJU_c09050 | CLRAG_13980 |
| | | 4.1.3.12 | | | |
| 20 | 2-keto-3-deoxy-phosphogluconate aldolase | 4.1.2.14, | CAETHG_3254 | CLJU_c11630 | CLRAG_25070 |
| | | 4.1.3.16, | | | |
| | | 4.1.1.3 | | | |
| 21 | 2-phosphosulfolactate phosphatase | | CAETHG_2017 | CLJU_c41880 | CLRAG_04990 |
| 22 | 2,3,4,5-tetrahydropyridine-2,6-dicarboxylate N- | 2.3.1.89 | CAETHG_1357 | CLJU_c34610 | CLRAG_14690 |
| | acetyltransferase | | | | |
| 23 | 23S rRNA m(2)A-2503 methyltransferase | | CAETHG_3342 | CLJU_c12600 | CLRAG_11200 |
| 24 | 3-dehydroquinate dehydratase | 4.2.1.10 | CAETHG_0871 | CLJU_c28760 | CLRAG_34840 |
| 25 | 3-dehydroquinate synthase | 4.2.3.4, | CAETHG_0908 | CLJU_c29160 | CLRAG_35160 |
| | | 4.6.1.3 | | | |
| 26 | 3-deoxy-D-arabinoheptulosonate-7-phosphate | 2.5.1.54, | CAETHG_0910 | CLJU_c29180 | CLRAG_35180 |
| | synthase | 4.1.2.15 | | | |
| 27 | 3-deoxy-D-arabinoheptulosonate-7-phosphate | 2.5.1.54, | CAETHG_3578 | CLJU_c14780 | CLRAG_20330 |
| | synthase | 4.1.2.15 | | | |
| 28 | 3-hydroxyacyl-[acyl-carrier-protein] dehydratase | 4.2.1.61, | CAETHG_2043 | CLJU_c42130 | CLRAG_05240 |
| | | 4.2.1.58, | | | |
| | | 2.3.1.86, | | | |
| | | 4.2.1.59, | | | |
| | | 4.2.1.60, | | | |
| | | 2.3.1.85, | | | |
| | | 4.2.1.0 | | | |
| 29 | 3-hydroxyacyl-CoA dehydrogenase | 1.1.1.157 | CAETHG_0420, | CLJU_c37300, | CLRAG_17610 |
| | | | CAETHG_1586 | CLJU_c23560 | |
| 30 | 3-isopropylmalate dehydratase, large subunit | 4.2.1.33 | CAETHG_3000 | CLJU_c09060 | CLRAG_13970 |
| 31 | 3-isopropylmalate/(R)-2-methylmalate | 4.2.1.33 | CAETHG_3001 | CLJU_c09070 | CLRAG_13960 |
| | dehydratase small subunit | | | | |
| 32 | 3-isopropylmalate dehydrogenase | 1.1.1.85, | CAETHG_1795, | CLJU_c39500, | CLRAG_13950 |
| | | | CAETHG_3002 | CLJU_c09080 | |
| 33 | 3-oxoacyl-[acyl-carrier-protein] reductase | 2.3.1.85, | CAETHG_1392, | CLJU_c42160, | CLRAG_26180 |
| | | 2.3.1.86, | CAETHG_2046 | CLJU_c34940 | |
| | | 1.1.1.100, | | | |
| | | 1.1.1.0, | | | |
| 34 | 3-oxoacyl-[acyl-carrier-protein] synthase II | 2.3.1.0, | CAETHG_2045 | CLJU_c42150 | CLRAG_05260 |
| | | 2.3.1.41, | | | |
| | | 2.3.1.180, | | | |
| | | 2.3.1.86, | | | |
| | | 2.3.1.38, | | | |
| | | 2.3.1.85, | | | |
| | | 2.3.1.179 | | | |
| 35 | 3-oxoacyl-[acyl-carrier-protein] synthase-3 | 2.3.1.0, | CAETHG_2050 | CLJU_c42190 | CLRAG_05300 |
| | | 2.3.1.41, | | | |
| | | 2.3.1.180, | | | |
| | | 2.3.1.86, | | | |
| | | 2.3.1.38, | | | |
| | | 2.3.1.85, | | | |
| | | 2.3.1.179 | | | |
| 36 | 3-phosphoshikimate 1-carboxyvinyltransferase | 2.5.1.19 | CAETHG_0907 | CLJU_c29150 | CLRAG_35150 |
| 37 | 5′-nucleotidase/3′-nucleotidase/ | 3.1.3.5 | CAETHG_1371 | CLJU_c34740 | CLRAG_14800 |
| | exopolyphosphatase | | | | |
| 38 | SSU ribosomal protein S10P | | CAETHG_1948 | CLJU_c41050 | CLRAG_23240 |
| 39 | SSU ribosomal protein S12P | | CAETHG_1952 | CLJU_c41090 | CLRAG_23280 |
| 40 | small subunit ribosomal protein S13 | | CAETHG_1923 | CLJU_c40800 | CLRAG_22990 |
| 41 | small subunit ribosomal protein S19 | | CAETHG_1943 | CLJU_c41000 | CLRAG_23190 |
| 42 | small subunit ribosomal protein S3 | | CAETHG_1941 | CLJU_c40980 | CLRAG_23170 |
| 43 | small subunit ribosomal protein S4 | | CAETHG_1921 | CLJU_c40780 | CLRAG_22970 |
| 44 | small subunit ribosomal protein S5 | | CAETHG_1930 | CLJU_c40870 | CLRAG_23060 |
| 45 | SSU ribosomal protein S6P | | CAETHG_2105 | CLJU_c42790 | CLRAG_25600 |
| 46 | small subunit ribosomal protein S7 | | CAETHG_1951 | CLJU_c41080 | CLRAG_23270 |
| 47 | small subunit ribosomal protein S8 | | CAETHG_1933 | CLJU_c40900 | CLRAG_23090 |
| 48 | 4-amino-4-deoxychorismate lyase | 4.1.3.38 | CAETHG_1508 | CLJU_c36000 | CLRAG_06500 |
| 49 | 4-aminobutyrate aminotransferase/(S)-3-amino- | 2.6.1.19 | CAETHG_0129 | CLJU_c20470 | CLRAG_19550 |
| | 2-methylpropionate transaminase | | | | |
| 50 | 4-diphosphocytidyl-2-C-methyl-D-erythritol kinase | 2.7.1.148 | CAETHG_2316 | CLJU_c02110 | CLRAG_27710 |
| 51 | 4-hydroxy-3-methylbut-2-en-1-yl diphosphate | | CAETHG_3393 | CLJU_c13100 | CLRAG_10690 |
| | synthase | | | | |
| 52 | 4-hydroxy-3-methylbut-2-enyl diphosphate | 1.17.1.2 | CAETHG_0218 | CLJU_c21320 | CLRAG_30880 |
| | reductase | | | | |
| 53 | 4-hydroxythreonine-4-phosphate dehydrogenase | 1.1.1.262 | CAETHG_2447 | CLJU_c03850 | CLRAG_28920 |
| 54 | 5-(carboxyamino)imidazole ribonucleotide mutase | 4.1.1.21 | CAETHG_2948 | CLJU_c08540 | CLRAG_07950 |
| 55 | 5-formyltetrahydrofolate cyclo-ligase | 6.3.3.2 | CAETHG_0286 | CLJU_c21900 | CLRAG_31440 |
| 56 | Vitamin B12 dependent methionine synthase | | CAETHG_2959 | CLJU_c08650 | CLRAG_07840 |
| | activation region | | | | |
| 57 | large subunit ribosomal protein L1 | | CAETHG_1958 | CLJU_c41150 | CLRAG_23340 |
| 58 | large subunit ribosomal protein L18 | | CAETHG_1931 | CLJU_c40880 | CLRAG_23070 |
| 59 | large subunit ribosomal protein L2 | | CAETHG_1944 | CLJU_c41010 | CLRAG_23200 |
| 60 | large subunit ribosomal protein L23 | | CAETHG_1945 | CLJU_c41020 | CLRAG_23210 |
| 61 | large subunit ribosomal protein L3 | | CAETHG_1947 | CLJU_c41040 | CLRAG_23230 |
| 62 | large subunit ribosomal protein L31 | | CAETHG_2328 | CLJU_c02230 | CLRAG_27830 |
| 63 | large subunit ribosomal protein L35 | | CAETHG_1345 | CLJU_c34450 | CLRAG_14530 |
| 64 | large subunit ribosomal protein L5 | | CAETHG_1935 | CLJU_c40920 | CLRAG_23110 |
| 65 | large subunit ribosomal protein L6 | | CAETHG_1932 | CLJU_c40890 | CLRAG_23080 |
| 66 | large subunit ribosomal protein L7/L12 | | CAETHG_1956 | CLJU_c41130 | CLRAG_23320 |
| 67 | 6-phosphofructokinase | 2.7.1.11, | CAETHG_0648, | CLJU_c03250, | CLRAG_18670 |
| | | 2.7.1.145, | CAETHG_2439 | CLJU_c25790 | |
| | | 2.7.1.144, | | | |
| | | 2.7.1.56 | | | |
| 68 | 6,7-dimethyl-8-ribityllumazine synthase | 2.5.1.9 | CAETHG_0304 | CLJU_c22060 | CLRAG_31580 |
| 69 | chaperonin GroEL | | CAETHG_1572 | CLJU_c37190 | CLRAG_36630 |
| 70 | ATP-binding cassette, subfamily B | | CAETHG_3619 | CLJU_c15170 | CLRAG_24180 |
| 71 | acetaldehyde dehydrogenase | 1.2.1.10 | CAETHG_1819, | CLJU_c39730, | CLRAG_21980 |
| | | | CAETHG_3287 | CLJU_c11960 | |
| 72 | acetaldehyde dehydrogenase/alcohol | 1.1.1.1, | CAETHG_3747, | CLJU_c16520, | CLRAG_33310 |
| | dehydrogenase | 1.1.1.72, | CAETHG_3748 | CLJU_c16510 | |
| | | 1.1.1.21, | | | |
| | | 1.1.1.2 | | | |
| 73 | acetate kinase | 2.7.2.1, | CAETHG_3359 | CLJU_c12780 | CLRAG_11030 |
| | | 2.7.2.15 | | | |
| 74 | acetolactate synthase-1/2/3 large subunit | 2.2.1.6, | CAETHG_1740 | CLJU_c38920 | CLRAG_21100 |
| | | 4.1.3.18 | | | |
| 75 | acetolactate synthase, large subunit | 2.2.1.6, | CAETHG_0124 | CLJU_c20420 | CLRAG_25870 |
| | | 4.1.1.1, | | | |
| | | 4.1.3.18, | | | |
| | | 1.2.4.1 | | | |
| 76 | acetolactate synthase, large subunit | 2.2.1.6, | CAETHG_0406 | CLJU_c23420 | CLRAG_01330 |
| | | 4.1.1.1, | | | |
| | | 4.1.3.18, | | | |
| | | 1.2.4.1 | | | |
| 77 | acetolactate synthase, small subunit | 2.2.1.6, | CAETHG_0125 | CLJU_c20430 | CLRAG_25860 |
| | | 4.1.1.1, | | | |
| | | 4.1.3.18, | | | |
| | | 1.2.4.1 | | | |
| 78 | acetyl-CoA carboxylase carboxyl transferase | 6.4.1.2 | CAETHG_2040 | CLJU_c42100 | CLRAG_05210 |
| | subunit alpha | | | | |
| 79 | acetyl-CoA carboxylase carboxyl transferase | 6.4.1.2 | CAETHG_2041 | CLJU_c42110 | CLRAG_05220 |
| | subunit beta | | | | |
| 80 | acetylornithine/N-succinyldiaminopimelate | 2.6.1.11 | CAETHG_0238 | CLJU_c21510 | CLRAG_31070 |
| | aminotransferase | | | | |
| 81 | aconitate hydratase | | CAETHG_0478 | CLJU_c24200 | CLRAG_24890 |
| 82 | ACT domain-containing protein | | CAETHG_0917 | CLJU_c29240 | CLRAG_35250 |
| 83 | FMN-dependent NADH-azoreductase | | CAETHG_0583 | CLJU_c25150 | CLRAG_03490 |
| 84 | Adenine deaminase | | CAETHG_0460 | CLJU_c23940 | CLRAG_17220 |
| 85 | Adenine deaminase | 3.5.4.2 | CAETHG_0681 | CLJU_c26120 | CLRAG_04200 |
| 86 | Adenine deaminase | 3.5.4.2 | CAETHG_0989 | CLJU_c29900 | CLRAG_35900 |
| 87 | adenine phosphoribosyltransferase | 2.4.2.7, | CAETHG_1270 | CLJU_c33720 | CLRAG_24560 |
| | | 2.4.2.8 | | | |
| 88 | adenosine deaminase | 3.5.4.4 | CAETHG_0825 | CLJU_c28280 | CLRAG_34360 |
| 89 | alpha-ribazole phosphatase | | CAETHG_1462 | CLJU_c35540 | CLRAG_06070 |
| 90 | adenosylcobinamide kinase/adenosylcobinamide- | 2.7.7.62, | CAETHG_1460 | CLJU_c35520 | CLRAG_06050 |
| | phosphate guanylyltransferase | 2.7.1.156 | | | |
| 91 | adenosylcobyric acid synthase (glutamine- | | CAETHG_1130 | CLJU_c32020 | CLRAG_02650 |
| | hydrolysing) | | | | |
| 92 | S-adenosylmethionine decarboxylase | 4.1.1.50 | CAETHG_0217 | CLJU_c21310 | CLRAG_30870 |
| 93 | Adenylate kinase | 2.7.4.11, | CAETHG_1926 | CLJU_c40830 | CLRAG_23020 |
| | | 2.7.4.3 | | | |
| 94 | adenylosuccinate lyase | 4.3.2.2 | CAETHG_3420 | CLJU_c13370 | CLRAG_10420 |
| 95 | Adenylosuccinate synthetase | 6.3.4.4 | CAETHG_2059 | CLJU_c42350 | CLRAG_05460 |
| 96 | ADP-ribose pyrophosphatase | 3.6.1.13 | CAETHG_3214 | CLJU_c11240 | CLRAG_12220 |
| 97 | carbon-monoxide dehydrogenase small subunit | 1.1.1.204, | CAETHG_0424 | CLJU_c23600 | CLRAG_17570 |
| | | 1.17.1.4 | | | |
| 98 | carbon-monoxide dehydrogenase medium subunit | 1.1.1.204, | CAETHG_0425 | CLJU_c23610 | CLRAG_17560 |
| | | 1.17.1.4 | | | |
| 99 | agmatine deiminase | 3.5.3.12 | CAETHG_2074 | CLJU_c42490 | CLRAG_09010 |
| 100 | accessory gene regulator B | | CAETHG_0843 | CLJU_c28480, | CLRAG_34560 |
| | | | | CLJU_c27530 | |
| 101 | alanine racemase | 5.1.1.1 | CAETHG_1140 | CLJU_c32120, | CLRAG_02750 |
| | | | | CLJU_c12010, | |
| | | | | CLJU_c40390 | |
| 102 | alanyl-tRNA synthetase | | CAETHG_3297 | CLJU_c12150 | CLRAG_11650 |
| 103 | Cysteine-rich domain-containing protein | | CAETHG_0470 | CLJU_c24120, | CLRAG_17130 |
| | | | | CLJU_c24040 | |
| 104 | aldehyde oxidoreductase | 2.3.1.169 | CAETHG_0471 | CLJU_c24050, | CLRAG_17120 |
| | | | | CLJU_c24130 | |
| 105 | aldose 1-epimerase | 5.1.3.3 | CAETHG_2227 | CLJU_c01190 | CLRAG_30230 |
| 106 | Allophanate hydrolase subunit 1 | | CAETHG_0130 | CLJU_c20480 | CLRAG_19540 |
| 107 | biotin-dependent carboxylase uncharacterized | 3.5.1.54 | CAETHG_0131 | CLJU_c20490 | CLRAG_19530 |
| | domain-containing protein | | | | |
| 108 | acetolactate decarboxylase | 4.1.1.5 | CAETHG_2932 | CLJU_c08380 | CLRAG_08070 |
| 109 | alpha-N-arabinofuranosidase | 3.2.1.55 | CAETHG_2233 | CLJU_c01240 | CLRAG_30180 |
| 110 | amidophosphoribosyltransferase | 2.4.2.14 | CAETHG_2950 | CLJU_c08560 | CLRAG_07930 |
| 111 | polar amino acid transport system ATP-binding | | CAETHG_2759 | CLJU_c06690 | CLRAG_18490 |
| | protein | | | | |
| 112 | amino acid ABC transporter membrane protein, | | CAETHG_1212, | CLJU_c06680, | CLRAG_15160 |
| | PAAT family | | CAETHG_2758 | CLJU_c33140 | |
| 113 | amino acid ABC transporter substrate-binding | | CAETHG_0569, | CLJU_c06670, | CLRAG_17770 |
| | protein, PAAT family | | CAETHG_2757 | CLJU_c25010 | |
| 114 | amino acid ABC transporter substrate-binding | | CAETHG_1211 | CLJU_c33130 | CLRAG_15170 |
| | protein, PAAT family | | | | |
| 115 | Amino acid transporter | | CAETHG_0009, | CLJU_c19320, | CLRAG_33220 |
| | | | CAETHG_3736 | CLJU_c16420 | |
| 116 | basic amino acid/polyamine antiporter, APA family | | CAETHG_0058 | CLJU_c19780 | CLRAG_39360 |
| 117 | amino acid/polyamine/organocation transporter, | | CAETHG_0165 | CLJU_c20800 | CLRAG_19200 |
| | APC superfamily | | | | |
| 118 | amino acid/polyamine/organocation transporter, | | CAETHG_0231, | CLJU_c21450, | CLRAG_31010 |
| | APC superfamily | | CAETHG_3020 | CLJU_c09260 | |
| 119 | basic amino acid/polyamine antiporter, APA family | | CAETHG_0407, | CLJU_c23440, | CLRAG_01320 |
| | | | CAETHG_0408 | CLJU_c23430 | |
| 120 | Amino acid transporter | | CAETHG_0483, | CLJU_c08730, | CLRAG_24920 |
| | | | CAETHG_2967 | CLJU_c24250 | |
| 121 | amino acid/polyamine/organocation transporter, | | CAETHG_0491 | CLJU_c24320 | CLRAG_24990 |
| | APC superfamily | | | | |
| 122 | amino acid/polyamine/organocation transporter, | | CAETHG_1802, | CLJU_c07120, | CLRAG_21780 |
| | APC superfamily | | CAETHG_2803 | CLJU_c39570 | |
| 123 | amino acid/polyamine/organocation transporter, | | CAETHG_2547, | CLJU_c04760, | CLRAG_38130 |
| | APC superfamily | | CAETHG_2548 | CLJU_c04750 | |
| 124 | amino acid/polyamine/organocation transporter, APC superfamily | | CAETHG_3898 | CLJU_c17900 | CLRAG_00730 |
| 125 | para-aminobenzoate synthetase component 1 | 2.6.1.85 | CAETHG_1509 | CLJU_c36010 | CLRAG_06510 |
| 126 | aminomethyltransferase | | CAETHG_0476 | CLJU_c24180 | CLRAG_24850 |
| 127 | aminopeptidase | | CAETHG_3684 | CLJU_c15760 | CLRAG_32920 |
| 128 | ammonium transporter | | CAETHG_2467 | CLJU_c04040 | CLRAG_29120 |
| 129 | anaerobic sulfite reductase subunit A | | CAETHG_0442 | CLJU_c23770 | CLRAG_17400 |
| 130 | Dissimilatory sulfite reductase (desulfoviridin), | 1.8.7.1 | CAETHG_1629 | CLJU_c37920 | CLRAG_37310 |
| | alpha and beta subunits | | | | |
| 131 | anaerobic sulfite reductase subunit B | | CAETHG_0441 | CLJU_c23760 | CLRAG_17410 |
| 132 | anaerobic sulfite reductase subunit C | | CAETHG_0440 | CLJU_c23750 | CLRAG_17420 |
| 133 | carbon-monoxide dehydrogenase catalytic subunit | 1.2.7.4 | CAETHG_3005 | CLJU_c09110 | CLRAG_13910 |
| 134 | carbon-monoxide dehydrogenase iron sulfur | | CAETHG_3004 | CLJU_c09100 | CLRAG_13920 |
| | subunit | | | | |
| 135 | Pyridine nucleotide-disulphide oxidoreductase | | CAETHG_3003 | CLJU_c09090 | CLRAG_13930 |
| 136 | anthranilate phosphoribosyltransferase | 2.4.2.18 | CAETHG_3703 | CLJU_c16090 | CLRAG_33060 |
| 137 | anthranilate synthase component 1 | 4.1.3.27 | CAETHG_3701 | CLJU_c16070 | CLRAG_33040 |
| 138 | para-aminobenzoate synthetase component 2 | 2.6.1.85, | CAETHG_1510, | CLJU_c16080, | CLRAG_06520 |
| | | 4.1.3.27 | CAETHG_3702 | CLJU_c36020 | |
| 139 | anti-anti-sigma regulatory factor, SpollAA | | CAETHG_1295 | CLJU_c33970 | CLRAG_14120 |
| 140 | anti-sigma-28 factor, FlgM family | | CAETHG_3044 | CLJU_c09490 | CLRAG_13610 |
| 141 | arginase | 3.5.3.1 | CAETHG_0290 | CLJU_c21930 | CLRAG_31480 |
| 142 | Arginine/lysine/ornithine decarboxylase | 4.1.1.18, | CAETHG_2244 | CLJU_c01380 | CLRAG_27040 |
| | | 4.1.1.19 | | | |
| 143 | argininosuccinate lyase | 4.3.2.1 | CAETHG_2762 | CLJU_c06710 | CLRAG_18510 |
| 144 | argininosuccinate synthase | 6.3.4.5 | CAETHG_2761 | CLJU_c06700 | CLRAG_18500 |
| 145 | arginyl-tRNA synthetase | | CAETHG_0257 | CLJU_c21700 | CLRAG_31290 |
| 146 | arsenite-transporting ATPase | | CAETHG_3665 | CLJU_c15660 | CLRAG_32720 |
| 147 | asparagine synthase (glutamine-hydrolysing) | 6.3.5.4 | CAETHG_0753 | CLJU_c26720 | CLRAG_08590 |
| 148 | asparagine synthase (glutamine-hydrolysing) | 6.3.5.4 | CAETHG_3879 | CLJU_c17710 | CLRAG_01020 |
| 149 | asparaginyl-tRNA synthetase | | CAETHG_2033 | CLJU_c42030 | CLRAG_05140 |
| 150 | aspartate aminotransferase | 2.6.1.23, | CAETHG_0215 | CLJU_c21290 | CLRAG_30850 |
| | | 2.6.1.1 | | | |
| 151 | aspartate aminotransferase | 2.6.1.23, | CAETHG_3417 | CLJU_c13340 | CLRAG_10450 |
| | | 2.6.1.1 | | | |
| 152 | aspartate ammonia-lyase | 4.2.1.2, | CAETHG_2062 | CLJU_c42370 | CLRAG_05490 |
| | | 4.3.1.1, | | | |
| | | 3.5.1.38 | | | |
| 153 | aspartate ammonia-lyase | 4.2.1.2, | CAETHG_2479 | CLJU_c04170 | CLRAG_26890 |
| | | 4.3.1.1, | | | |
| | | 3.5.1.38 | | | |
| 154 | aspartate carbamoyltransferase | 2.1.3.2 | CAETHG_1481 | CLJU_c35730 | CLRAG_06260 |
| 155 | aspartate carbamoyltransferase regulatory subunit | 2.1.3.2 | CAETHG_1480 | CLJU_c35720 | CLRAG_06250 |
| 156 | aspartate kinase | | CAETHG_1187 | CLJU_c32890 | CLRAG_15440 |
| 157 | aspartate kinase | | CAETHG_1690 | CLJU_c38320 | CLRAG_20790 |
| 158 | aspartate racemase | | CAETHG_0938 | CLJU_c29440 | CLRAG_35430 |
| 159 | aspartate semialdehyde dehydrogenase | | CAETHG_1353 | CLJU_c34570 | CLRAG_14650 |
| 160 | asparaginyl-tRNA synthetase | | CAETHG_2765 | CLJU_c06740 | CLRAG_18540 |
| 161 | aspartyl aminopeptidase | | CAETHG_2066 | CLJU_c42410 | CLRAG_05550 |
| 162 | aspartyl-tRNA synthetase | | CAETHG_1264 | CLJU_c33660 | CLRAG_24620 |
| 163 | aspartyl/glutamyl-tRNA(Asn/Gln) | | CAETHG_1553 | CLJU_c36920 | CLRAG_36450 |
| | amidotransferase subunit A | | | | |
| 164 | aspartyl/glutamyl-tRNA(Asn/Gln) | | CAETHG_1552 | CLJU_c36910 | CLRAG_36440 |
| | amidotransferase subunit B | | | | |
| 165 | aspartyl/glutamyl-tRNA(Asn/Gln) | | CAETHG_1554 | CLJU_c36930 | CLRAG_36460 |
| | amidotransferase subunit C | | | | |
| 166 | ATP phosphoribosyltransferase | 2.4.2.17 | CAETHG_3258 | CLJU_c11670 | CLRAG_11870 |
| 167 | ATP phosphoribosyltransferase regulatory subunit | 2.4.2.17 | CAETHG_3257 | CLJU_c11660 | CLRAG_11880 |
| 168 | F-type H+-transporting ATPase subunit a | 3.6.3.14 | CAETHG_2343 | CLJU_c02370 | CLRAG_27980 |
| 169 | ATP synthase F0 subcomplex B subunit | 3.6.3.14 | CAETHG_2345 | CLJU_c02390 | CLRAG_28000 |
| 170 | ATP synthase F0 subcomplex C subunit | 3.6.3.14 | CAETHG_2344 | CLJU_c02380 | CLRAG_27990 |
| 171 | ATP synthase F1 subcomplex alpha subunit | 3.6.3.14 | CAETHG_2347 | CLJU_c02410 | CLRAG_28020 |
| 172 | ATP synthase F1 subcomplex beta subunit | 3.6.3.14 | CAETHG_2349 | CLJU_c02430 | CLRAG_28040 |
| 173 | ATP synthase F1 subcomplex delta subunit | 3.6.3.14 | CAETHG_2346 | CLJU_c02400 | CLRAG_28010 |
| 174 | ATP-dependent Clp protease ATP-binding subunit | | CAETHG_1471 | CLJU_c35630 | CLRAG_06160 |
| | ClpX | | | | |
| 175 | ATP-dependent Clp protease ATP-binding subunit | | CAETHG_0538 | CLJU_c24730 | CLRAG_18090 |
| | ClpA | | | | |
| 176 | ATP-dependent Clp protease, protease subunit | | CAETHG_1192, | CLJU_c35640, | CLRAG_15390 |
| | | | CAETHG_1472 | CLJU_c32940 | |
| 177 | DNA helicase-2/ATP-dependent DNA helicase | | CAETHG_1559 | CLJU_c36980 | CLRAG_36520 |
| | PcrA | | | | |
| 178 | ATP-dependent DNA helicase RecG | | CAETHG_3351 | CLJU_c12700 | CLRAG_11110 |
| 179 | ATP-dependent DNA helicase RecQ | | CAETHG_0594 | CLJU_c25260 | CLRAG_03610 |
| 180 | cell division protease FtsH | | CAETHG_1987 | CLJU_c41530 | CLRAG_04660 |
| 181 | ATP-dependent Clp protease adaptor protein ClpS | | CAETHG_0539 | CLJU_c24740 | CLRAG_18080 |
| 182 | ATP-dependent Lon protease | | CAETHG_2097 | CLJU_c42720 | CLRAG_25530 |
| 183 | Predicted ATP-dependent protease | | CAETHG_3140 | CLJU_c10500 | CLRAG_12870 |
| 184 | ATP-dependent RNA helicase DbpA | | CAETHG_2474 | CLJU_c04110 | CLRAG_26940 |
| 185 | DNA-binding protein HU-beta | | CAETHG_1996 | CLJU_c41670 | CLRAG_04800 |
| 186 | peptide chain release factor 2 | | CAETHG_2365 | CLJU_c02640 | CLRAG_28240 |
| 187 | translation initiation factor IF-2 | | CAETHG_3398 | CLJU_c13150 | CLRAG_10640 |
| 188 | CubicO group peptidase, beta-lactamase class C | | CAETHG_1431, | CLJU_c08840, | CLRAG_05740 |
| | family | | CAETHG_2979 | CLJU_c35230 | |
| 189 | phosphoribosylaminoimidazolecarboxamide | 3.5.4.10, | CAETHG_0319 | CLJU_c22210 | CLRAG_31790 |
| | formyltransferase/IMP cyclohydrolase | 2.1.2.3 | | | |
| 190 | BirA family transcriptional regulator, biotin operon | 6.3.4.14 | CAETHG_0747 | CLJU_c26660 | CLRAG_08530 |
| | repressor/biotin-[acetyl-CoA-carboxylase] ligase | | | | |
| 191 | riboflavin kinase/FMN adenylyltransferase | 2.7.7.2, | CAETHG_3402 | CLJU_c13190 | CLRAG_10600 |
| | | 2.7.1.26 | | | |
| 192 | mannose-1-phosphate guanylyltransferase/ | 2.7.7.22 | CAETHG_2615, | CLJU_c05540, | CLRAG_38830 |
| | mannose-6-phosphate isomerase | | CAETHG_2637 | CLJU_c05310 | |
| 193 | flagellar biosynthetic protein FliR/FlhB | | CAETHG_3126 | CLJU_c10360 | CLRAG_13010 |
| 194 | dihydroneopterin aldolase/2-amino-4-hydroxy-6- | 4.1.2.25, | CAETHG_2732 | CLJU_c06370 | CLRAG_30460 |
| | hydroxymethyldihydropteridine diphosphokinase | 2.7.6.3 | | | |
| 195 | bifunctional UDP-N-acetylglucosamine | 2.3.1.4, | CAETHG_2007 | CLJU_c41780 | CLRAG_04910 |
| | pyrophosphorylase/Glucosamine-1-phosphate N- | 2.7.7.23, | | | |
| | acetyltransferase | 2.3.1.157 | | | |
| 196 | phosphoribosylaminoimidazolecarboxamide | 3.5.4.10, | CAETHG_2953 | CLJU_c08590 | CLRAG_07900 |
| | formyltransferase/IMP cyclohydrolase | 2.1.2.3 | | | |
| 197 | tRNA nucleotidyltransferase (CCA-adding enzyme) | | CAETHG_3219 | CLJU_c11280 | CLRAG_12170 |
| 198 | Biotin carboxylase C-terminal domain-containing | | CAETHG_0127 | CLJU_c20450 | CLRAG_19570 |
| | protein | | | | |
| 199 | acetyl-CoA carboxylase, biotin carboxylase subunit | 6.3.4.14 | CAETHG_2042 | CLJU_c42120 | CLRAG_05230 |
| 200 | branched-chain amino acid:cation transporter, | | CAETHG_3882 | CLJU_c17740 | CLRAG_00980 |
| | LIVCS family | | | | |
| 201 | branched-chain amino acid aminotransferase | 2.6.1.67, | CAETHG_3032 | CLJU_c09370 | CLRAG_13730 |
| | | 2.6.1.42, | | | |
| | | 2.6.1.6 | | | |
| 202 | carbamate kinase | 1.3.99.1, | CAETHG_0445, | CLJU_c23800, | CLRAG_17370 |
| | | 2.7.2.2 | CAETHG_3025 | CLJU_c09300 | |
| 203 | carbamate kinase | 2.7.2.2 | CAETHG_2081 | CLJU_c42550 | CLRAG_09000 |
| 204 | carbamoyl-phosphate synthase large subunit | 6.3.5.5 | CAETHG_0589, | CLJU_c04410, | CLRAG_03560 |
| | | | CAETHG_2510 | CLJU_c25210 | |
| 205 | carbamoyl-phosphate synthase small subunit | 6.3.5.5 | CAETHG_0590, | CLJU_c04400, | CLRAG_03570 |
| | | | CAETHG_2508 | CLJU_c25220 | |
| 206 | carbohydrate ABC transporter substrate-binding protein, CUT1 | | CAETHG_1309 | CLJU_c34110 | CLRAG_14260 |
| | family | | | | |
| 207 | carbohydrate ABC transporter substrate-binding | | CAETHG_1464 | CLJU_c35560 | CLRAG_06090 |
| | protein, CUT1 family | | | | |
| 208 | multiple sugar transport system substrate-binding | | CAETHG_2301 | CLJU_c01980 | CLRAG_27580 |
| | protein | | | | |
| 209 | carbon-monoxide dehydrogenase catalytic subunit | 1.2.7.4 | CAETHG_3899 | CLJU_c17910 | CLRAG_00720 |
| 210 | carbon starvation protein | | CAETHG_1590, | CLJU_c37350, | CLRAG_36780 |
| | | | CAETHG_1591 | CLJU_c37340 | |
| 211 | carbon storage regulator, CsrA | | CAETHG_3064 | CLJU_c09690 | CLRAG_13480 |
| 212 | 4-carboxymuconolactone decarboxylase | 4.1.1.44 | CAETHG_0634 | CLJU_c25650 | CLRAG_03850 |
| 213 | CDP-diacylglycerol--glycerol-3-phosphate 3- | 2.7.8.5 | CAETHG_3410 | CLJU_c13270 | CLRAG_10520 |
| | phosphatidyltransferase | | | | |
| 214 | cell division protein FtsA | | CAETHG_3311 | CLJU_c12290 | CLRAG_11510 |
| 215 | cell division protein FtsA | | CAETHG_3846 | CLJU_c17330 | CLRAG_29250 |
| 216 | cell division protein FtsQ | | CAETHG_3151 | CLJU_c10610 | CLRAG_12730 |
| 217 | cell division transport system permease protein | | CAETHG_2423 | CLJU_c03080 | CLRAG_28690 |
| 218 | cell division protein FtsZ | | CAETHG_3312 | CLJU_c12300 | CLRAG_11500 |
| 219 | ATP-dependent Clp protease ATP-binding subunit | | CAETHG_2717 | CLJU_c06170 | CLRAG_07450 |
| | ClpB | | | | |
| 220 | two-component system, chemotaxis family, sensor | | CAETHG_3038 | CLJU_c09430 | CLRAG_13670 |
| | kinase CheA | | | | |
| 221 | chemotaxis protein MotA | | CAETHG_2251 | CLJU_c01450 | CLRAG_27110 |
| 222 | purine-binding chemotaxis protein CheW | | CAETHG_3034 | CLJU_c09390 | CLRAG_13710 |
| 223 | chemotaxis protein CheD | | CAETHG_3035 | CLJU_c09400 | CLRAG_13700 |
| 224 | purine-binding chemotaxis protein CheW | | CAETHG_3041 | CLJU_c09460 | CLRAG_13640 |
| 225 | chemotaxis protein CheC | | CAETHG_3039 | CLJU_c09440 | CLRAG_13660 |
| 226 | chemotaxis protein methyltransferase CheR | | CAETHG_3037 | CLJU_c09420 | CLRAG_13680 |
| 227 | two-component system, chemotaxis family, | | CAETHG_3036 | CLJU_c09410 | CLRAG_13690 |
| | response regulator CheB | | | | |
| 228 | chloramphenicol O-acetyltransferase type A | | CAETHG_0663 | CLJU_c25940 | CLRAG_04080 |
| 229 | chorismate mutase | 4.2.1.91, | CAETHG_0905 | CLJU_c29130 | CLRAG_35130 |
| | | 4.2.1.51 | | | |
| 230 | chorismate synthase | 4.2.3.5 | CAETHG_0906 | CLJU_c29140 | CLRAG_35140 |
| 231 | chromosomal replication initiator protein DnaA | | CAETHG_2124 | CLJU_c00010 | CLRAG_25790 |
| 232 | [citrate (pro-3S)-lyase] ligase | 2.3.3.1 | CAETHG_1898 | CLJU_c40550 | CLRAG_22740 |
| 233 | citrate lyase subunit gamma (acyl carrier protein) | 2.3.3.1 | CAETHG_1901, | CLJU_c40580 | CLRAG_22770 |
| | | | CAETHG_2481 | | |
| 234 | citrate lyase subunit alpha/citrate CoA- | 2.3.3.1 | CAETHG_1899, | CLJU_c40560 | CLRAG_22750 |
| | transferase | | CAETHG_2483 | | |
| 235 | citrate lyase subunit beta/citryl-CoA lyase | 2.3.3.1 | CAETHG_1900, | CLJU_c40570 | CLRAG_22760 |
| | | | CAETHG_2482 | | |
| 236 | CO-methylating acetyl-CoA synthase corrinoid | 2.3.1.169 | CAETHG_1610 | CLJU_c37570 | CLRAG_36980 |
| | iron-sulfur protein large subunit precursor | | | | |
| 237 | CO-methylating acetyl-CoA synthase corrinoid | 2.3.1.169 | CAETHG_1611 | CLJU_c37580 | CLRAG_36990 |
| | iron-sulfur protein small subunit precursor/acetyl- | | | | |
| | CoA decarbonylase/synthase delta subunit | | | | |
| 238 | CO-methylating acetyl-CoA synthase precursor/ | 2.3.1.169 | CAETHG_1608 | CLJU_c37550 | CLRAG_36960 |
| | acetyl-CoA decarbonylase/synthase beta subunit | | | | |
| 239 | cob(l)yrinic acid a,c-diamide adenosyltransferase | 2.5.1.17 | CAETHG_1110 | CLJU_c31820 | CLRAG_02450 |
| 240 | adenosylcobinamide-phosphate synthase | | CAETHG_1129 | CLJU_c32010 | CLRAG_02640 |
| 241 | cobalamin-5′-phosphate synthase | 2.7.8.26 | CAETHG_1461 | CLJU_c35530 | CLRAG_06060 |
| 242 | cobalt-precorrin 3 C17-methyltransferase | 2.1.1.131 | CAETHG_1114 | CLJU_c31860 | CLRAG_02490 |
| 243 | cobalt-precorrin 4 C11-methyltransferase | 2.1.1.133 | CAETHG_1116 | CLJU_c31880 | CLRAG_02510 |
| 244 | cobalt-precorrin 5A acetaldehyde-lyase | | CAETHG_1115 | CLJU_c31870 | CLRAG_02500 |
| 245 | cobalt-precorrin 5B C1-methyltransferase | | CAETHG_1120 | CLJU_c31920 | CLRAG_02550 |
| 246 | cobalt-precorrin-6B (C15)-methyltransferase | 2.1.1.132 | CAETHG_1118 | CLJU_c31900 | CLRAG_02530 |
| 247 | precorrin-8X methylmutase/cobalt-precorrin 8 | 5.4.1.2 | CAETHG_1121 | CLJU_c31930 | CLRAG_02560 |
| | methylmutase | | | | |
| 248 | precorrin-6A/cobalt-precorrin-6A reductase | 1.3.1.54 | CAETHG_1112 | CLJU_c31840 | CLRAG_02470 |
| 249 | CobW/HypB/UreG, nucleotide-binding domain | | CAETHG_0147 | CLJU_c20640 | CLRAG_19340 |
| 250 | cold-shock DNA-binding protein family | | CAETHG_0027, | CLJU_c19580, | CLRAG_39610 |
| | | | CAETHG_0035 | CLJU_c19500 | |
| 251 | competence/damage-inducible protein cinA | | CAETHG_1770 | CLJU_c39260 | CLRAG_21470 |
| 252 | condensin subunit ScpA | | CAETHG_3220 | CLJU_c11290 | CLRAG_12160 |
| 253 | segregation and condensation protein B | | CAETHG_3221 | CLJU_c11300 | CLRAG_12150 |
| 254 | condensin subunit Smc | | CAETHG_3367 | CLJU_c12850 | CLRAG_10950 |
| 255 | Cu+-exporting ATPase | | CAETHG_0557 | CLJU_c24900 | CLRAG_17880 |
| 256 | 16S rRNA (cytidinel402-2′-O)-methyltransferase | | CAETHG_2254 | CLJU_c01480 | CLRAG_27140 |
| 257 | CTP synthase | 6.3.4.2 | CAETHG_2325 | CLJU_c02200 | CLRAG_27800 |
| 258 | superoxide dismutase, Cu—Zn family | 1.15.1.1 | CAETHG_0977 | CLJU_c29780 | CLRAG_35780 |
| 259 | cyanophycin synthetase | | CAETHG_2315 | CLJU_c02100 | CLRAG_27700 |
| 260 | cyanophycinase | | CAETHG_2314 | CLJU_c02090 | CLRAG_27690 |
| 261 | Cellobiose phosphorylase | | CAETHG_1687 | CLJU_c38300 | CLRAG_20770 |
| 262 | cyclopropane-fatty-acyl-phospholipid synthase | | CAETHG_0840 | CLJU_c28420 | CLRAG_34500 |
| 263 | cystathionine gamma-lyase | | CAETHG_0498 | CLJU_c24380 | CLRAG_25120 |
| 264 | cysteine desulfurase | | CAETHG_0833 | CLJU_c28360 | CLRAG_34440 |
| 265 | Selenocysteine lyase/Cysteine desulfurase | | CAETHG_1227 | CLJU_c33280 | CLRAG_14980 |
| 266 | cysteine desulfurase family protein | | CAETHG_1218 | CLJU_c33190 | CLRAG_15070 |
| 267 | cysteine synthase A | | CAETHG_0497 | CLJU_c24370 | CLRAG_25110 |
| 268 | cysteine synthase A | 2.5.1.47, | CAETHG_1776 | CLJU_c39310 | CLRAG_21520 |
| | | 2.5.1.65, | | | |
| | | 4.2.99.8 | | | |
| 269 | Cysteine synthase | | CAETHG_2922 | CLJU_c08270 | CLRAG_08120 |
| 270 | cysteinyl-tRNA synthetase | | CAETHG_0170 | CLJU_c20850 | CLRAG_19150 |
| 271 | cysteinyl-tRNA synthetase | | CAETHG_1968 | CLJU_c41270 | CLRAG_23460 |
| 272 | dCMP deaminase | 3.5.4.12 | CAETHG_2339 | CLJU_c02330 | CLRAG_27940 |
| 273 | cytidine deaminase | | CAETHG_3921 | CLJU_c18120 | CLRAG_00540 |
| 274 | cytidylate kinase | 2.7.4.14, | CAETHG_0219 | CLJU_c21330 | CLRAG_30890 |
| | | 2.7.1.48 | | | |
| 275 | cytosine deaminase | 3.5.4.1 | CAETHG_4058 | CLJU_c19230 | CLRAG_39840 |
| 276 | D-3-phosphoglycerate dehydrogenase | 1.1.1.95 | CAETHG_2211 | CLJU_c00970 | CLRAG_19610 |
| 277 | D-alanine-D-alanine ligase | 6.3.2.4 | CAETHG_1139 | CLJU_c32110 | CLRAG_02740 |
| 278 | D-alanyl-D-alanine carboxypeptidase | | CAETHG_2836 | CLJU_c07440 | CLRAG_32250 |
| 279 | D-alanyl-D-alanine carboxypeptidase (penicillin- | | CAETHG_3218 | CLJU_c11270 | CLRAG_12180 |
| | binding protein 5/6) | | | | |
| 280 | D-alanyl-D-alanine carboxypeptidase | | CAETHG_3425 | CLJU_c13410 | CLRAG_10380 |
| 281 | D-alanyl-D-alanine carboxypeptidase | | CAETHG_3680 | CLJU_c15720 | CLRAG_32880 |
| 282 | D-alanyl-D-alanine carboxypeptidase | | CAETHG_3224 | CLJU_c11330 | CLRAG_12120 |
| 283 | D-glucarate dehydratase | 4.2.1.40 | CAETHG_0817 | CLJU_c28130, | CLRAG_09100 |
| | | | | CLJU_c28170 | |
| 284 | g-D-glutamyl-meso-diaminopimelate peptidase | | CAETHG_2777 | CLJU_c06860 | CLRAG_18700 |
| 285 | dihydropyrimidinase | | CAETHG_0444 | CLJU_c23790 | CLRAG_17380 |
| 286 | D-serine/D-alanine/glycine:proton symporter, AAT family | | CAETHG_2928 | CLJU_c08330 | CLRAG_08080 |
| 287 | xylose isomerase | 5.3.1.5 | CAETHG_3932 | CLJU_c18240 | CLRAG_00370 |
| 288 | EDD domain protein, DegV family | | CAETHG_3256 | CLJU_c11650 | CLRAG_11890 |
| 289 | DNA-binding transcriptional regulator, MerR family | | CAETHG_3906 | CLJU_c17970 | CLRAG_00660 |
| 290 | deoxyribose-phosphate aldolase | | CAETHG_3922 | CLJU_c18130 | CLRAG_00530 |
| 291 | dUTP pyrophosphatase | 3.6.1.19, | CAETHG_3104 | CLJU_c10140 | CLRAG_13230 |
| | | 3.6.1.23 | | | |
| 292 | dephospho-CoA kinase | 2.7.1.24 | CAETHG_1258 | CLJU_c33600 | CLRAG_24680 |
| 293 | diacylglycerol kinase (ATP) | 2.7.1.107 | CAETHG_2904 | CLJU_c08090 | CLRAG_08260 |
| 294 | diaminohydroxyphosphoribosylaminopyrimidine | 3.5.4.26, | CAETHG_0307 | CLJU_c22090 | CLRAG_31610 |
| | deaminase/5-amino-6-(5- | 1.1.1.193 | | | |
| | phosphoribosylamino)uracil reductase | | | | |
| 295 | diaminopimelate decarboxylase | 4.1.1.20 | CAETHG_1688 | CLJU_c38310 | CLRAG_20780 |
| 296 | diaminopimelate epimerase | 5.1.1.7 | CAETHG_3166 | CLJU_c10760 | CLRAG_12580 |
| 297 | diaminopropionate ammonia-lyase | | CAETHG_0451 | CLJU_c23860 | CLRAG_17310 |
| 298 | diguanylate cyclase (GGDEF) domain-containing | | CAETHG_1216 | CLJU_c33170 | CLRAG_15120 |
| | protein | | | | |
| 299 | dihydrodipicolinate reductase | 1.3.1.26 | CAETHG_1351 | CLJU_c34550 | CLRAG_14630 |
| 300 | dihydrodipicolinate reductase | 1.3.1.26 | CAETHG_3914 | CLJU_c18050 | CLRAG_00600 |
| 301 | 4-hydroxy-tetrahydrodipicolinate synthase | 4.2.1.52 | CAETHG_0823 | CLJU_c28230 | CLRAG_09180 |
| 302 | 4-hydroxy-tetrahydrodipicolinate synthase | 4.2.1.52 | CAETHG_1352, | CLJU_c04300, | CLRAG_14640 |
| | | | CAETHG_2498 | CLJU_c34560 | |
| 303 | dihydrofolate reductase | | CAETHG_0509 | CLJU_c24490 | CLRAG_30080 |
| 304 | dihydrolipoamide dehydrogenase | 1.8.1.4 | CAETHG_1613 | CLJU_c37600 | CLRAG_37010 |
| 305 | dihydroorotase | 3.5.2.3 | CAETHG_1585 | CLJU_c37290 | CLRAG_36730 |
| 306 | dihydroorotate dehydrogenase (NAD+) catalytic | 1.3.3.1, | CAETHG_1477 | CLJU_c35690 | CLRAG_06220 |
| | subunit | 1.3.99.11 | | | |
| 307 | dihydroorotate dehydrogenase electron transfer | 1.3.3.1, | CAETHG_1478 | CLJU_c35700 | CLRAG_06230 |
| | subunit | 1.3.99.11 | | | |
| 308 | dihydropteroate synthase | 2.5.1.15 | CAETHG_2729 | CLJU_c06340 | CLRAG_30490 |
| 309 | dihydropyrimidinase | 3.5.2.2 | CAETHG_1496 | CLJU_c35890 | CLRAG_06390 |
| 310 | dihydropyrimidine dehydrogenase (NAD+) subunit | 1.3.1.2 | CAETHG_1494 | CLJU_c35870 | CLRAG_06370 |
| | PreA | | | | |
| 311 | dihydroxy-acid dehydratase | 4.2.1.9 | CAETHG_0123 | CLJU_c20410 | CLRAG_25880 |
| 312 | 16S rRNA (adeninel518-N6/adenine1519-N6)- | | CAETHG_2279 | CLJU_c01770 | CLRAG_27370 |
| | dimethyltransferase | | | | |
| 313 | DNA gyrase subunit A | | CAETHG_2130 | CLJU_c00070 | CLRAG_25850 |
| 314 | topoisomerase-4 subunit A | | CAETHG_3014 | CLJU_c09200 | CLRAG_13830 |
| 315 | DNA gyrase subunit B | | CAETHG_2129 | CLJU_c00060 | CLRAG_25840 |
| 316 | DNA helicase/exodeoxyribonuclease V, subunit A | | CAETHG_1215 | CLJU_c33160 | CLRAG_15130 |
| 317 | DNA helicase/exodeoxyribonuclease V, subunit B | | CAETHG_2788 | CLJU_c06980 | CLRAG_18820 |
| 318 | DNA ligase (NAD+) | | CAETHG_1558 | CLJU_c36970 | CLRAG_36510 |
| 319 | DNA mismatch repair protein MutS2 | | CAETHG_1338 | CLJU_c34380 | CLRAG_14460 |
| 320 | DNA mismatch repair protein MutL | | CAETHG_0209 | CLJU_c21230 | CLRAG_30810 |
| 321 | DNA mismatch repair protein MutS | | CAETHG_0210 | CLJU_c21240 | CLRAG_30820 |
| 322 | DNA polymerase I | | CAETHG_1259 | CLJU_c33610 | CLRAG_24670 |
| 323 | DNA polymerase-3 subunit alpha | | CAETHG_2438 | CLJU_c03240 | CLRAG_28840 |
| 324 | DNA polymerase-3 subunit alpha | | CAETHG_1073 | CLJU_c30690 | CLRAG_16120 |
| 325 | DNA polymerase-3 subunit beta | | CAETHG_2125 | CLJU_c00020 | CLRAG_25800 |
| 326 | DNA polymerase-3 subunit gamma/tau | | CAETHG_2199 | CLJU_c00850 | CLRAG_19730 |
| 327 | DNA polymerase III, delta subunit | | CAETHG_2882 | CLJU_c07890 | CLRAG_25370 |
| 328 | DNA polymerase-4 | | CAETHG_0189 | CLJU_c21040 | CLRAG_18980 |
| 329 | DNA polymerase-3 subunit delta′ | | CAETHG_2247 | CLJU_c01410 | CLRAG_27070 |
| 330 | DNA primase | | CAETHG_2916 | CLJU_c08210 | CLRAG_08180 |
| 331 | DNA repair protein RadA/Sms | | CAETHG_1973 | CLJU_c41320 | CLRAG_23510 |
| 332 | DNA replication and repair protein RadC | | CAETHG_2813 | CLJU_c07210 | CLRAG_26630 |
| 333 | DNA replication and repair protein RecF | | CAETHG_2127 | CLJU_c00040 | CLRAG_25820 |
| 334 | DNA replication and repair protein RecN | | CAETHG_3209 | CLJU_c11200 | CLRAG_12260 |
| 335 | DNA replication and repair protein RecO | | CAETHG_2906 | CLJU_c08110 | CLRAG_08240 |
| 336 | DNA replication and repair protein RecR | | CAETHG_2201 | CLJU_c00870 | CLRAG_19710 |
| 337 | DNA topoisomerase-3 | | CAETHG_0360 | CLJU_c22980 | CLRAG_01800 |
| 338 | DNA topoisomerase-3 | | CAETHG_0411 | CLJU_c23470 | CLRAG_17640 |
| 339 | DNA topoisomerase-1 | | CAETHG_3383 | CLJU_c13000 | CLRAG_10790 |
| 340 | topoisomerase-4 subunit B | | CAETHG_3013 | CLJU_c09190 | CLRAG_13840 |
| 341 | DNA segregation ATPase FtsK/SpoIIIE, S-DNA-T | | CAETHG_3408 | CLJU_c13250 | CLRAG_10540 |
| | family | | | | |
| 342 | endonuclease-3 | | CAETHG_1771 | CLJU_c39270 | CLRAG_21480 |
| 343 | DNA-directed RNA polymerase subunit alpha | | CAETHG_1920 | CLJU_c40770 | CLRAG_22960 |
| 344 | DNA-directed RNA polymerase subunit beta | | CAETHG_1955 | CLJU_c41120 | CLRAG_23310 |
| 345 | DNA-directed RNA polymerase subunit beta′ | | CAETHG_1954 | CLJU_c41110 | CLRAG_23300 |
| 346 | DNA-directed RNA polymerase subunit omega | | CAETHG_3335 | CLJU_c12530 | CLRAG_11270 |
| 347 | dTDP-4-dehydrorhamnose 3,5-epimerase | 5.1.3.13 | CAETHG_2619, | CLJU_c05350, | CLRAG_06720 |
| | | | CAETHG_2641 | CLJU_c05580 | |
| 348 | dTDP-4-dehydrorhamnose reductase | 1.1.1.133, | CAETHG_2618, | CLJU_c05570, | CLRAG_06710 |
| | | | CAETHG_2640 | CLJU_c05340 | |
| 349 | dTDP-glucose 4,6-dehydratase | 4.2.1.46 | CAETHG_2616, | CLJU_c05550, | CLRAG_06690 |
| | | | CAETHG_2638 | CLJU_c05320 | |
| 350 | glucose-1-phosphate thymidylyltransferase | 2.7.7.33, | CAETHG_2617, | CLJU_c05330, | CLRAG_06700 |
| | | 2.7.7.24 | CAETHG_2639 | CLJU_c05560 | |
| 351 | electron transfer flavoprotein alpha subunit | | CAETHG_0116, | CLJU_c13890, | CLRAG_25950 |
| | apoprotein | | CAETHG_3472 | CLJU_c20340 | |
| 352 | electron transfer flavoprotein alpha subunit | | CAETHG_0245 | CLJU_c21580 | CLRAG_31170 |
| | apoprotein | | | | |
| 353 | electron transfer flavoprotein alpha subunit | | CAETHG_1785 | CLJU_c39400 | CLRAG_21610 |
| | apoprotein | | | | |
| 354 | electron transfer flavoprotein beta subunit | | CAETHG_0115, | CLJU_c13880, | CLRAG_25960 |
| | | | CAETHG_3471 | CLJU_c20330 | |
| 355 | electron transfer flavoprotein beta subunit | | CAETHG_0246 | CLJU_c21590 | CLRAG_31180 |
| 356 | electron transfer flavoprotein beta subunit | | CAETHG_1786 | CLJU_c39410 | CLRAG_21620 |
| 357 | electron transport complex protein RnfA | 1.18.1.3 | CAETHG_3231 | CLJU_c11400 | CLRAG_12050 |
| 358 | electron transport complex protein RnfD | 1.18.1.3 | CAETHG_3228 | CLJU_c11370 | CLRAG_12080 |
| 359 | electron transport complex protein RnfE | 1.18.1.3 | CAETHG_3230 | CLJU_c11390 | CLRAG_12060 |
| 360 | elongation factor P | | CAETHG_3190 | CLJU_c11010 | CLRAG_12450 |
| 361 | elongation factor Ts | | CAETHG_3386 | CLJU_c13030 | CLRAG_10760 |
| 362 | elongation factor Tu | | CAETHG_1949, | CLJU_c41200, | CLRAG_23390 |
| | | | CAETHG_1963 | CLJU_c41060 | |
| 363 | Endonuclease IV | | CAETHG_0108 | CLJU_c20270 | CLRAG_26020 |
| 364 | 2-enoate reductase | | CAETHG_0983 | CLJU_c29840 | CLRAG_35850 |
| 365 | 2,4-dienoyl-CoA reductase | | CAETHG_1079 | CLJU_c30750 | CLRAG_16190 |
| 366 | 2-enoate reductase | | CAETHG_1247 | CLJU_c33480 | CLRAG_32290 |
| 367 | enolase | 4.2.1.11 | CAETHG_1756 | CLJU_c39110 | CLRAG_21260 |
| 368 | enoyl-[acyl-carrier protein] reductase II | | CAETHG_2049 | CLJU_c42180 | CLRAG_05290 |
| 369 | Alcohol dehydrogenase, class IV | 1.1.1.1, | CAETHG_1813 | CLJU_c39670 | CLRAG_21920 |
| | | 1.1.1.72, | | | |
| | | 1.1.1.21, | | | |
| | | 1.1.1.2 | | | |
| 370 | Alcohol dehydrogenase, class IV | 1.1.1.1, | CAETHG_3279 | CLJU_c11880 | CLRAG_03030 |
| | | 1.1.1.72, | | | |
| | | 1.1.1.21, | | | |
| | | 1.1.1.2 | | | |
| 371 | excinuclease ABC subunit A | | CAETHG_2427 | CLJU_c03120 | CLRAG_28730 |
| 372 | Excinuclease ABC subunit B | | CAETHG_2426 | CLJU_c03110 | CLRAG_28720 |
| 373 | Excinuclease ABC subunit C | | CAETHG_2432 | CLJU_c03170 | CLRAG_28780 |
| 374 | Exodeoxyribonuclease I subunit D | | CAETHG_0112 | CLJU_c20310 | CLRAG_25980 |
| 375 | exodeoxyribonuclease V alpha subunit | | CAETHG_2359 | CLJU_c02590 | CLRAG_28190 |
| 376 | Exodeoxyribonuclease VII large subunit | | CAETHG_3202 | CLJU_c11130 | CLRAG_12330 |
| 377 | Exodeoxyribonuclease VII small subunit | | CAETHG_3203 | CLJU_c11140 | CLRAG_12320 |
| 378 | single-stranded-DNA-specific exonuclease | | CAETHG_3018 | CLJU_c09240 | CLRAG_13790 |
| 379 | F-type H+-transporting ATPase subunit epsilon | 3.6.3.14 | CAETHG_2350 | CLJU_c02440 | CLRAG_28050 |
| 380 | F-type H+-transporting ATPase subunit gamma | 3.6.3.14 | CAETHG_2348 | CLJU_c02420 | CLRAG_28030 |
| 381 | ATP synthase protein I | 3.6.3.14 | CAETHG_2342 | CLJU_c02360 | CLRAG_27970 |
| 382 | [FeFe] hydrogenase, group A | 1.12.1.4, | CAETHG_2798, | CLJU_c17280, | CLRAG_18920 |
| | | 1.1.99.33 | CAETHG_3841 | CLJU_c07070 | |
| 383 | electron transport protein HydN | | CAETHG_0083, | CLJU_c20030, | CLRAG_32510 |
| | | | CAETHG_3840 | CLJU_c17270 | |
| 384 | ferrous iron transport protein A | | CAETHG_3480 | CLJU_c13970 | CLRAG_09280 |
| 385 | 4Fe—4S dicluster domain-containing protein | | CAETHG_2250 | CLJU_c01440 | CLRAG_27100 |
| 386 | ferredoxin | | CAETHG_2285 | CLJU_c01820 | CLRAG_27420 |
| 387 | Fur family transcriptional regulator, ferric uptake | | CAETHG_3301 | CLJU_c12190 | CLRAG_11610 |
| | regulator | | | | |
| 388 | ferritin | | CAETHG_0026 | CLJU_c19490 | CLRAG_39620 |
| 389 | ferrous iron transport protein B | | CAETHG_3481 | CLJU_c13980 | CLRAG_09290 |
| 390 | ferrous iron transport protein A | | CAETHG_3479 | CLJU_c13960 | CLRAG_09270 |
| 391 | flagellar assembly protein FliH | | CAETHG_3114 | CLJU_c10240 | CLRAG_13130 |
| 392 | flagellar basal-body rod protein FlgB | | CAETHG_3109 | CLJU_c10190 | CLRAG_13180 |
| 393 | flagellar basal-body rod protein FlgG | | CAETHG_3134 | CLJU_c10440 | CLRAG_12930 |
| 394 | flagellar basal-body rod protein FlgG | | CAETHG_3135 | CLJU_c10450 | CLRAG_12920 |
| 395 | flagellar basal-body rod protein FlgC | | CAETHG_3110 | CLJU_c10200 | CLRAG_13170 |
| 396 | flagellar FliJ protein | | CAETHG_3116 | CLJU_c10260 | CLRAG_13110 |
| 397 | flagellar biosynthesis protein FlhA | | CAETHG_3127 | CLJU_c10370 | CLRAG_13000 |
| 398 | flagellar biosynthesis protein FlhF | | CAETHG_3128 | CLJU_c10380 | CLRAG_12990 |
| 399 | flagellar FliL protein | | CAETHG_3122 | CLJU_c10320 | CLRAG_13050 |
| 400 | flagellar biosynthetic protein FliP | | CAETHG_3124 | CLJU_c10340 | CLRAG_13030 |
| 401 | flagellar biosynthetic protein FliQ | | CAETHG_3125 | CLJU_c10350 | CLRAG_13020 |
| 402 | flagellar hook-associated protein 1 FlgK | | CAETHG_3046 | CLJU_c09510 | CLRAG_13590 |
| 403 | flagellar hook-associated protein 2 | | CAETHG_3056 | CLJU_c09610 | CLRAG_13530 |
| 404 | flagellar hook-basal body complex protein FliE | | CAETHG_3111 | CLJU_c10210 | CLRAG_13160 |
| 405 | flagellar M-ring protein FliF | | CAETHG_3112 | CLJU_c10220 | CLRAG_13150 |
| 406 | flagellar motor switch protein FliM | | CAETHG_3042 | CLJU_c09470 | CLRAG_13630 |
| 407 | flagellar motor switch protein FliG | | CAETHG_3113 | CLJU_c10230 | CLRAG_13140 |
| 408 | flagellar protein FliS | | CAETHG_3052 | CLJU_c09570 | CLRAG_13540 |
| 409 | flagellin | | CAETHG_3108 | CLJU_c10180 | CLRAG_13190 |
| 410 | foldase protein PrsA | | CAETHG_2000 | CLJU_c41710 | CLRAG_04840 |
| 411 | dihydrofolate synthase/folylpolyglutamate | 6.3.2.12, | CAETHG_1365 | CLJU_c34680 | CLRAG_14760 |
| | synthase | 6.3.2.17 | | | |
| 412 | formate dehydrogenase major subunit | 1.2.1.43, | CAETHG_2790, | CLJU_c08930, | CLRAG_18840 |
| | | 1.1.99.33 | CAETHG_2988 | CLJU_c06990 | |
| 413 | Formate-tetrahydrofolate ligase | 3.5.4.9, | CAETHG_1618 | CLJU_c37650 | CLRAG_37060 |
| | | 6.3.4.3 | | | |
| 414 | formiminotetrahydrofolate cyclodeaminase | 4.3.1.4 | CAETHG_1728 | CLJU_c38800 | CLRAG_21060 |
| 415 | formiminoglutamase | 3.5.3.8 | CAETHG_0228 | CLJU_c21420 | CLRAG_30980 |
| 416 | Formiminotetrahydrofolate cyclodeaminase | 4.3.1.4 | CAETHG_0230 | CLJU_c21440 | CLRAG_31000 |
| 417 | formylmethanofuran dehydrogenase subunit E | | CAETHG_2994 | CLJU_c38060, | CLRAG_07510 |
| | | | | CLJU_c09000 | |
| 418 | Formiminotetrahydrofolate cyclodeaminase | 3.5.4.9, | CAETHG_1617 | CLJU_c37640 | CLRAG_37050 |
| | | 4.3.1.4, | | | |
| | | 6.3.4.3 | | | |
| 419 | formyltetra hydrofolate-dependent | 2.1.2.2 | CAETHG_2952 | CLJU_c08580 | CLRAG_07910 |
| | phosphoribosylglycinamide formyltransferase | | | | |
| 420 | 1-phosphofructokinase | 2.7.1.11, | CAETHG_0143 | CLJU_c20600 | CLRAG_19380 |
| | | 2.7.1.145, | | | |
| | | 2.7.1.144, | | | |
| | | 2.7.1.56 | | | |
| 421 | fructose-1,6-bisphosphatase-3 | 3.1.3.11 | CAETHG_0897 | CLJU_c29050 | CLRAG_35050 |
| 422 | probable phosphoglycerate mutase | | CAETHG_0464 | CLJU_c23980 | CLRAG_17180 |
| 423 | fructose-bisphosphate aldolase, class II | 4.1.2.13 | CAETHG_2184 | CLJU_c00660 | CLRAG_19910 |
| 424 | fructose-bisphosphate aldolase | 4.1.2.13 | CAETHG_2382 | CLJU_c02810 | CLRAG_28410 |
| 425 | fumarase, class I alpha subunit | 4.2.1.81, | CAETHG_1903 | CLJU_c40600 | CLRAG_22790 |
| | | 4.2.1.2, | | | |
| | | 4.2.1.32 | | | |
| 426 | fumarate hydratase subunit beta | 4.2.1.81, | CAETHG_1902 | CLJU_c40590 | CLRAG_22780 |
| | | 4.2.1.2, | | | |
| | | 4.2.1.32 | | | |
| 427 | 2-keto-4-pentenoate hydratase/2-oxohepta-3-ene- | | CAETHG_0592 | CLJU_c25240 | CLRAG_03590 |
| | 1,7-dioic acid hydratase (catechol pathway) | | | | |
| 428 | gamma-glutamyltransferase 2. Threonine | 2.3.2.2, | CAETHG_4037 | CLJU_c19030 | CLRAG_40010 |
| | peptidase. MEROPS family T03 | 3.4.11.4 | | | |
| 429 | geranylgeranyl diphosphate synthase, type II | 2.5.1.29, | CAETHG_3204 | CLJU_c11150 | CLRAG_12310 |
| | | 2.5.1.1, | | | |
| | | 2.5.1.10 | | | |
| 430 | spore protease | | CAETHG_2884 | CLJU_c07910 | CLRAG_25390 |
| 431 | diguanylate cyclase (GGDEF) domain-containing | | CAETHG_0847 | CLJU_c28520 | CLRAG_34600 |
| | protein | | | | |
| 432 | diguanylate cyclase (GGDEF) domain-containing | | CAETHG_0678 | CLJU_c26090 | CLRAG_04180 |
| | protein | | | | |
| 433 | gluconate permease GntP | | CAETHG_2180, | CLJU_c11600, | CLRAG_19960 |
| | | | CAETHG_3251 | CLJU_c00620 | |
| 434 | tRNA uridine 5-carboxymethylaminomethyl | | CAETHG_2117 | CLJU_c42910 | CLRAG_25720 |
| | modification enzyme | | | | |
| 435 | glucose-6-phosphate isomerase | 5.3.1.9, | CAETHG_1568 | CLJU_c37130 | CLRAG_36590 |
| | | 5.1.3.15 | | | |
| 436 | glutamate 5-kinase | 2.7.2.11 | CAETHG_2697 | CLJU_c05990 | CLRAG_07220 |
| 437 | glutamate formiminotransferase | 2.1.2.5 | CAETHG_0237 | CLJU_c21500 | CLRAG_31060 |
| 438 | conserved hypothetical protein | | CAETHG_1906 | CLJU_c40630 | CLRAG_22820 |
| 439 | Glutamate mutase subunit E | | CAETHG_1905 | CLJU_c40620 | CLRAG_22810 |
| 440 | glutamate mutase subunit S | | CAETHG_1907 | CLJU_c40640 | CLRAG_22830 |
| 441 | glutamate N-acetyltransferase | 2.3.1.1, | CAETHG_0240 | CLJU_c21530 | CLRAG_31090 |
| | | 2.3.1.35 | | | |
| 442 | glutamate racemase | 5.1.1.3 | CAETHG_2023 | CLJU_c41940 | CLRAG_05050 |
| 443 | glutamate synthase (NADPH/NADH) small chain | 1.4.1.13, | CAETHG_1580 | CLJU_c37240 | CLRAG_36680 |
| | | 1.6.99.3 | | | |
| 444 | glutamate synthase (NADPH/NADH) large chain | 1.4.1.13 | CAETHG_3850 | CLJU_c17370 | CLRAG_29210 |
| 445 | glutamate synthase (NADH) small subunit | 1.4.1.13 | CAETHG_3851 | CLJU_c17380 | CLRAG_29200 |
| 446 | glutamate-1-semialdehyde 2,1-aminomutase | 5.4.3.8 | CAETHG_2521 | CLJU_c04490 | CLRAG_37850 |
| 447 | glutamate-5-semialdehyde dehydrogenase | 1.2.1.41 | CAETHG_2698 | CLJU_c06000 | CLRAG_07230 |
| 448 | glutamine synthetase | 6.3.1.2 | CAETHG_2024 | CLJU_c41950 | CLRAG_05060 |
| 449 | glucosamine-fructose-6-phosphate | 2.6.1.16 | CAETHG_1885 | CLJU_c40420 | CLRAG_22610 |
| | aminotransferase (isomerizing) | | | | |
| 450 | NAD+ synthase (glutamine-hydrolysing) | 6.3.1.5, | CAETHG_2782 | CLJU_c06920 | CLRAG_18760 |
| | | 6.3.5.1 | | | |
| 451 | glutaminyl-tRNA synthetase | | CAETHG_0755 | CLJU_c26740 | CLRAG_08610 |
| 452 | glutamyl-tRNA reductase | 1.2.1.70 | CAETHG_2520 | CLJU_c04480 | CLRAG_37840 |
| 453 | glutamyl-tRNA synthetase | 6.1.1.17, | CAETHG_3423 | CLJU_c13390 | CLRAG_10400 |
| | | 6.1.1.24 | | | |
| 454 | Glutathionylspermidine synthase | | CAETHG_3949 | CLJU_c18420 | CLRAG_00260 |
| 455 | glyceraldehyde-3-phosphate dehydrogenase | 1.2.1.59, | CAETHG_1760, | CLJU_c13400, | CLRAG_21300 |
| | (NAD+) | 1.2.1.12, | CAETHG_3424 | CLJU_c39150 | |
| | | 1.2.1.72 | | | |
| 456 | glycerol 3-phosphate dehydrogenase (NAD(P)+) | 1.1.1.94, | CAETHG_3330 | CLJU_c12480 | CLRAG_11320 |
| | | 1.1.1.261, | | | |
| | | 1.1.1.8 | | | |
| 457 | glycerol-3-phosphate dehydrogenase | | CAETHG_1600 | CLJU_c37480 | CLRAG_36890 |
| 458 | Glycerophosphoryl diester phosphodiesterase | | CAETHG_0269 | CLJU_c21800 | CLRAG_31340 |
| | family protein | | | | |
| 459 | glycine dehydrogenase (decarboxylating) alpha | | CAETHG_0474 | CLJU_c24160 | CLRAG_24830 |
| | subunit | | | | |
| 460 | glycine dehydrogenase subunit 2 | | CAETHG_0473 | CLJU_c24150 | CLRAG_24820 |
| 461 | glycyl-tRNA synthetase | | CAETHG_1981 | CLJU_c41460 | CLRAG_04590 |
| 462 | GMP synthase (glutamine-hydrolysing) | 6.3.5.2 | CAETHG_1570 | CLJU_c37170 | CLRAG_36610 |
| 463 | GTP cyclohydrolase I | 3.5.4.16 | CAETHG_2730 | CLJU_c06350 | CLRAG_30480 |
| 464 | GTP pyrophosphokinase | 2.7.6.5 | CAETHG_1269 | CLJU_c33710 | CLRAG_24570 |
| 465 | guanine deaminase | | CAETHG_0462 | CLJU_c23960 | CLRAG_17200 |
| 466 | guanylate kinase | 2.7.4.8, | CAETHG_3334 | CLJU_c12520 | CLRAG_11280 |
| | | 2.7.4.12 | | | |
| 467 | molecular chaperone HtpG | | CAETHG_0057 | CLJU_c19770 | CLRAG_39370 |
| 468 | heat-inducible transcription repressor HrcA | | CAETHG_2889 | CLJU_c07960 | CLRAG_25440 |
| 469 | hemerythrin | | CAETHG_0273 | CLJU_c21830 | CLRAG_31370 |
| 470 | hemerythrin-like metal-binding domain protein | | CAETHG_1518 | CLJU_c36090 | CLRAG_06600 |
| 471 | hemolysin III | | CAETHG_1262 | CLJU_c33640 | CLRAG_24640 |
| 472 | heptaprenyl diphosphate synthase | 2.5.1.29, | CAETHG_3233 | CLJU_c11420 | CLRAG_12030 |
| | | 2.5.1.30, | | | |
| | | 2.5.1.33 | | | |
| 473 | histidine ammonia-lyase | 4.3.1.3 | CAETHG_1182 | CLJU_c32840 | CLRAG_15490 |
| 474 | histidine ammonia-lyase | 4.3.1.3 | CAETHG_0232 | CLJU_c21460 | CLRAG_31020 |
| 475 | histidinol dehydrogenase | 1.1.1.23 | CAETHG_3259 | CLJU_c11680 | CLRAG_11860 |
| 476 | histidinol-phosphatase (PHP family) | 3.1.3.15 | CAETHG_3272 | CLJU_c11810 | CLRAG_11730 |
| 477 | histidinol-phosphate aminotransferase | 2.6.1.9, | CAETHG_3263 | CLJU_c11720 | CLRAG_11820 |
| | | 2.6.1.58, | | | |
| | | 2.6.1.57, | | | |
| | | 2.6.1.5, | | | |
| | | 2.6.1.1 | | | |
| 478 | histidyl-tRNA synthetase | | CAETHG_1265 | CLJU_c33670 | CLRAG_24610 |
| 479 | Holliday junction DNA helicase subunit RuvA | | CAETHG_1281 | CLJU_c33830 | CLRAG_24450 |
| 480 | Holliday junction DNA helicase subunit RuvB | | CAETHG_1280 | CLJU_c33820 | CLRAG_24460 |
| 481 | holo-[acyl-carrier-protein] synthase | 2.7.8.7 | CAETHG_2415 | CLJU_c03000 | CLRAG_28610 |
| 482 | homocitrate synthase NifV | 4.1.3.21, | CAETHG_2575 | CLJU_c04980 | CLRAG_38370 |
| | | 2.3.3.14 | | | |
| 483 | homocitrate synthase NifV | | CAETHG_2574 | CLJU_c04970 | CLRAG_38360 |
| 484 | cysteine desulfurase | 4.1.99.1, | CAETHG_0403, | CLJU_c12110, | CLRAG_01360 |
| | | 4.4.1.8, | CAETHG_3293 | CLJU_c23390 | |
| | | 4.4.1.1 | | | |
| 485 | homoserine dehydrogenase | 1.1.1.3 | CAETHG_2807 | CLJU_c07150 | CLRAG_26690 |
| 486 | homoserine dehydrogenase | 1.1.1.3 | CAETHG_3099 | CLJU_c10090 | CLRAG_13280 |
| 487 | homoserine kinase | 2.7.1.39 | CAETHG_2808 | CLJU_c07160 | CLRAG_26680 |
| 488 | homoserine O-succinyltransferase | | CAETHG_0492 | CLJU_c24330 | CLRAG_25000 |
| 489 | Hpr(Ser) kinase/phosphatase | | CAETHG_0287 | CLJU_c21910 | CLRAG_31450 |
| 490 | Hydrogenase maturation protein HypC | | CAETHG_0371 | CLJU_c23080 | CLRAG_01730 |
| 491 | Hydrogenase maturation protein HypD | | CAETHG_0370 | CLJU_c23070 | CLRAG_01740 |
| 492 | Hydrogenase maturation protein, carbamoyl | | CAETHG_0369 | CLJU_c23060 | CLRAG_01750 |
| | dehydratase HypE | | | | |
| 493 | hydrogenase expression/formation protein HypE | 2.7.4.16 | CAETHG_1548 | CLJU_c36870 | CLRAG_36400 |
| 494 | Hydrogenase maturation protein, | | CAETHG_0372 | CLJU_c23090 | CLRAG_01720 |
| | carbamoyltransferase HypF | | | | |
| 495 | cobyrinic acid a,c-diamide synthase | 6.3.1.— | CAETHG_1123 | CLJU_c31950 | CLRAG_02580 |
| 496 | hypothetical protein | | CAETHG_1730 | CLJU_c38820 | CLRAG_08740 |
| 497 | Uncharacterized conserved protein YgbK, DUF1537 | | CAETHG_2185 | CLJU_c00670 | CLRAG_19900 |
| | family | | | | |
| 498 | hydroxyethylthiazole kinase | 2.7.1.50 | CAETHG_1203 | CLJU_c33050 | CLRAG_15280 |
| 499 | hydroxyethylthiazole kinase | 2.7.1.50 | CAETHG_1415 | CLJU_c35060 | CLRAG_26320 |
| 500 | hydroxymethylbilane synthase | 2.5.1.61, | CAETHG_1126 | CLJU_c31980 | CLRAG_02610 |
| | | 4.3.1.8 | | | |
| 501 | glycerate dehydrogenase | 1.1.1.95 | CAETHG_0004, | CLJU_c19280, | CLRAG_39780 |
| | | | CAETHG_1243 | CLJU_c33430 | |
| 502 | protein of unknown function (DUF4163) | | CAETHG_0010 | CLJU_c19330 | CLRAG_39760 |
| 503 | NADPH-dependent FMN reductase | | CAETHG_0012 | CLJU_c19350 | CLRAG_39740 |
| 504 | hypothetical protein | | CAETHG_0013 | CLJU_c19360 | CLRAG_39730 |
| 505 | DNA-binding transcriptional regulator, XRE-family | | CAETHG_0015 | CLJU_c19380 | CLRAG_39710 |
| | HTH domain | | | | |
| 506 | hypothetical protein | | CAETHG_0016 | CLJU_c19390 | CLRAG_39700 |
| 507 | rarD protein | | CAETHG_0020 | CLJU_c19430 | CLRAG_39680 |
| 508 | protein of unknown function (DUF1848) | | CAETHG_0021 | CLJU_c19440 | CLRAG_39670 |
| 509 | hypothetical protein | | CAETHG_0024 | CLJU_c19470 | CLRAG_39640 |
| 510 | hypothetical protein | | CAETHG_0045 | CLJU_c19680 | CLRAG_39460 |
| 511 | Hypothetical protein | | CAETHG_0050 | CLJU_c19730 | CLRAG_39410 |
| 512 | Uncharacterized conserved protein, contains | | CAETHG_0054 | CLJU_c19750 | CLRAG_39390 |
| | FIST_N domain | | | | |
| 513 | hypothetical protein | | CAETHG_0060 | CLJU_c19800 | CLRAG_39280 |
| 514 | Ala-tRNA(Pro) deacylase | | CAETHG_0063 | CLJU_c19830 | CLRAG_39250 |
| 515 | L-cysteine desulfidase | | CAETHG_0067 | CLJU_c19870 | CLRAG_39210 |
| 516 | hypothetical protein | | CAETHG_0074 | CLJU_c19940 | CLRAG_39120 |
| 517 | hypothetical protein | | CAETHG_0075, | CLJU_c02730, | CLRAG_39090 |
| | | | CAETHG_2375 | CLJU_c19950 | |
| 518 | putative GTP pyrophosphokinase | 2.7.6.5 | CAETHG_0076, | CLJU_c30620, | CLRAG_39080 |
| | | | CAETHG_1066 | CLJU_c19960 | |
| 519 | hypothetical protein | | CAETHG_0081, | CLJU_c20010, | CLRAG_29290 |
| | | | CAETHG_3842 | CLJU_c17290 | |
| 520 | MOSC domain containing protein | | CAETHG_0100, | CLJU_c20190, | CLRAG_29730 |
| | | | CAETHG_0572 | CLJU_c25040 | |
| 521 | DNA binding domain-containing protein, | | CAETHG_0106 | CLJU_c20250 | CLRAG_26040 |
| | excisionase family | | | | |
| 522 | transporter family protein | | CAETHG_0107 | CLJU_c20260 | CLRAG_26030 |
| 523 | hypothetical protein (DUF2334) | | CAETHG_0111 | CLJU_c20300 | CLRAG_25990 |
| 524 | hypothetical protein | | CAETHG_0126, | CLJU_c28630, | CLRAG_19580 |
| | | | CAETHG_0858 | CLJU_c20440 | |
| 525 | UPF0271 protein | | CAETHG_0133 | CLJU_c20510 | CLRAG_19510 |
| 526 | Protein of unknown function (DUF1097) | | CAETHG_0134 | CLJU_c20520 | CLRAG_19500 |
| 527 | Predicted metal-binding protein | | CAETHG_0136 | CLJU_c20540 | CLRAG_19460 |
| 528 | Protein of unknown function (DUF1638) | | CAETHG_0146 | CLJU_c20630 | CLRAG_19350 |
| 529 | hypothetical protein | | CAETHG_0148 | CLJU_c20650 | CLRAG_19330 |
| 530 | Predicted metal-binding protein | | CAETHG_0149 | CLJU_c20660 | CLRAG_19320 |
| 531 | protein of unknown function (DUF4445) | | CAETHG_0156 | CLJU_c20720 | CLRAG_19280 |
| 532 | hypothetical protein | | CAETHG_0167 | CLJU_c20820 | CLRAG_19180 |
| 533 | hypothetical protein | | CAETHG_0168 | CLJU_c20830 | CLRAG_19170 |
| 534 | Ion channel | | CAETHG_0171 | CLJU_c20860 | CLRAG_19140 |
| 535 | HutD protein | | CAETHG_0172 | CLJU_c20870 | CLRAG_19130 |
| 536 | Protein of unknown function (DUF1657) | | CAETHG_0174 | CLJU_c20890 | CLRAG_19120 |
| 537 | Uncharacterized membrane protein YcaP, DUF421 | | CAETHG_0175 | CLJU_c20900 | CLRAG_19110 |
| | family | | | | |
| 538 | Protein of unknown function (DUF1657) | | CAETHG_0179 | CLJU_c20950 | CLRAG_19070 |
| 539 | Protein of unknown function (DUF3006) | | CAETHG_0181 | CLJU_c20970 | CLRAG_19050 |
| 540 | hypothetical protein | | CAETHG_0187 | CLJU_c21020 | CLRAG_19000 |
| 541 | Cys-tRNA(Pro) deacylase | | CAETHG_0201 | CLJU_c21150 | CLRAG_30710 |
| 542 | uroporphyrinogen decarboxylase | | CAETHG_0204 | CLJU_c21180 | CLRAG_19410 |
| 543 | Hemerythrin-like domain-containing protein | | CAETHG_0243 | CLJU_c21560 | CLRAG_31140 |
| 544 | Nickel-dependent lactate racemase | | CAETHG_0247, | CLJU_c04620, | CLRAG_31190 |
| | | | CAETHG_2534 | CLJU_c21600 | |
| 545 | RNase_H superfamily protein | | CAETHG_0250 | CLJU_c21630 | CLRAG_31220 |
| 546 | HAD-superfamily subfamily IB hydrolase, | 3.1.3.3 | CAETHG_0251 | CLJU_c21640 | CLRAG_31230 |
| | TIGR01490 | | | | |
| 547 | Virus attachment protein pl2 family protein | | CAETHG_0254 | CLJU_c21670 | CLRAG_31260 |
| 548 | CBS domain-containing protein | | CAETHG_0255 | CLJU_c21680 | CLRAG_31270 |
| 549 | hypothetical protein | | CAETHG_0256 | CLJU_c21690 | CLRAG_31280 |
| 550 | putative membrane protein | | CAETHG_0265 | CLJU_c21770 | CLRAG_31310 |
| 551 | hypothetical protein | | CAETHG_0266 | CLJU_c21780 | CLRAG_31320 |
| 552 | Uncharacterized membrane protein YvlD, DUF360 | | CAETHG_0274 | CLJU_c21840 | CLRAG_31380 |
| | family | | | | |
| 553 | hypothetical protein | | CAETHG_0275 | CLJU_c21850 | CLRAG_31390 |
| 554 | protein of unknown function (DUF896) | | CAETHG_0288 | CLJU_c21920 | CLRAG_31460 |
| 555 | hypothetical protein | | CAETHG_0297 | CLJU_c21990 | CLRAG_31520 |
| 556 | hypothetical protein | | CAETHG_0299 | CLJU_c22010 | CLRAG_31540 |
| 557 | hypothetical protein | | CAETHG_0301 | CLJU_c22030 | CLRAG_31560 |
| 558 | Soluble P-type ATPase | | CAETHG_0317 | CLJU_c22190 | CLRAG_31770 |
| 559 | cobalt/nickel transport protein | | CAETHG_0320 | CLJU_c22220 | CLRAG_31800 |
| 560 | Xylose isomerase-like TIM barrel | | CAETHG_0322 | CLJU_c22240 | CLRAG_31820 |
| 561 | RND family efflux transporter, MFP subunit | | CAETHG_0324, | CLJU_c05950, | CLRAG_31840 |
| | | | CAETHG_2692 | CLJU_c22260 | |
| 562 | PucR C-terminal helix-turn-helix domain- | | CAETHG_0338 | CLJU_c22760 | CLRAG_02030 |
| | containing protein | | | | |
| 563 | L-2-amino-thiazoline-4-carboxylic acid hydrolase | | CAETHG_0343 | CLJU_c22810 | CLRAG_01980 |
| 564 | succinate dehydrogenase/fumarate reductase | 1.3.99.1 | CAETHG_0344 | CLJU_c22820 | CLRAG_01970 |
| | iron-sulfur subunit | | | | |
| 565 | Uncharacterized SAM-binding protein YcdF, | | CAETHG_0346 | CLJU_c22840 | CLRAG_01950 |
| | DUF218 family | | | | |
| 566 | hypothetical protein | | CAETHG_0347 | CLJU_c22850 | CLRAG_01940 |
| 567 | 3′,5′-cyclic AMP phosphodiesterase CpdA | | CAETHG_0348 | CLJU_c22860 | CLRAG_01930 |
| 568 | protein of unknown function (DUF326) | | CAETHG_0349 | CLJU_c22870 | CLRAG_01920 |
| 569 | CRISPR-associated protein Csh1 | | CAETHG_0359 | CLJU_c22970 | CLRAG_01810 |
| 570 | Nitrogenase component 1 type Oxidoreductase | | CAETHG_0366 | CLJU_c23030 | CLRAG_01780 |
| 571 | Nitrogenase component 1 type Oxidoreductase | | CAETHG_0367 | CLJU_c23040 | CLRAG_01770 |
| 572 | Nitrogenase component 1 type Oxidoreductase | | CAETHG_0373 | CLJU_c23100 | CLRAG_01710 |
| 573 | Putative multidrug resistance efflux transporter | | CAETHG_0376 | CLJU_c23130 | CLRAG_01680 |
| 574 | hypothetical protein | | CAETHG_0380 | CLJU_c23170 | CLRAG_01650 |
| 575 | 3D (Asp-Asp-Asp) domain-containing protein | | CAETHG_0383 | CLJU_c23200 | CLRAG_01530 |
| 576 | ABC-2 type transport system permease protein | | CAETHG_0389 | CLJU_c23260 | CLRAG_01470 |
| 577 | Ca-activated chloride channel family protein | | CAETHG_0395 | CLJU_c23310 | CLRAG_01430 |
| 578 | hypothetical protein | 2.1.1.45 | CAETHG_0396 | CLJU_c23320 | CLRAG_01420 |
| 579 | hypothetical protein | | CAETHG_0404 | CLJU_c23400 | CLRAG_01350 |
| 580 | ECF transporter S component, folate family | | CAETHG_0405 | CLJU_c23410 | CLRAG_01340 |
| 581 | Vitamin B12 dependent methionine synthase | | CAETHG_0409 | CLJU_c23450 | CLRAG_17660 |
| | activation region | | | | |
| 582 | hypothetical protein | | CAETHG_0428 | CLJU_c23640 | CLRAG_17530 |
| 583 | Protein of unknown function (DUF1116) | | CAETHG_0430 | CLJU_c23660 | CLRAG_17510 |
| 584 | Protein of unknown function (DUF2877) | | CAETHG_0432 | CLJU_c23680 | CLRAG_17490 |
| 585 | Uncharacterized protein, pyridoxamine 5′- | | CAETHG_0434, | CLJU_c24290, | CLRAG_17470 |
| | phosphate oxidase (PNPOx-like) family | | CAETHG_0487 | CLJU_c23700 | |
| 586 | C_GCAxxG_C_C family probable redox protein | | CAETHG_0468 | CLJU_c24090, | CLRAG_30590 |
| | | | | CLJU_c24020 | |
| 587 | hypothetical protein | | CAETHG_0469 | CLJU_c24110, | CLRAG_17140 |
| | | | | CLJU_c24030 | |
| 588 | hypothetical protein | 2.3.1.0, | CAETHG_0488 | CLJU_c24300 | CLRAG_24970 |
| | | 2.3.1.86, | | | |
| | | 2.3.1.39, | | | |
| | | 2.3.1.3, | | | |
| | | 2.3.1.2, | | | |
| | | 2.3.1.1, | | | |
| | | 2.3.1.85 | | | |
| 589 | Regulator of protease activity HflC, | | CAETHG_0490 | CLJU_c24310 | CLRAG_24980 |
| | stomatin/prohibitin superfamily | | | | |
| 590 | epoxyqueuosine reductase | | CAETHG_0495 | CLJU_c24350 | CLRAG_25090 |
| 591 | hypothetical protein | | CAETHG_0500 | CLJU_c24400 | CLRAG_25130 |
| 592 | hypothetical protein | | CAETHG_0508 | CLJU_c24480 | CLRAG_30090 |
| 593 | Uncharacterized membrane protein | | CAETHG_0510 | CLJU_c24500 | CLRAG_30070 |
| 594 | Anti-sigma factor N-terminus | | CAETHG_0512 | CLJU_c24520 | CLRAG_30050 |
| 595 | RNA polymerase sigma factor | | CAETHG_0513 | CLJU_c24530 | CLRAG_30040 |
| 596 | Permease of the drug/metabolite transporter | | CAETHG_0533 | CLJU_c24680 | CLRAG_18150 |
| | (DMT) superfamily | | | | |
| 597 | Methyltransferase domain-containing protein | | CAETHG_0535 | CLJU_c24700 | CLRAG_35650 |
| 598 | hypothetical protein | | CAETHG_0536 | CLJU_c24710 | CLRAG_18110 |
| 599 | Uncharacterized membrane protein | | CAETHG_0537 | CLJU_c24720 | CLRAG_18100 |
| 600 | Protein of unknown function (DUF2975) | | CAETHG_0546 | CLJU_c24810 | CLRAG_17970 |
| 601 | hypothetical protein | | CAETHG_0550 | CLJU_c24830 | CLRAG_17950 |
| 602 | DNA-binding transcriptional regulator, FrmR family | | CAETHG_0558 | CLJU_c24910 | CLRAG_17870 |
| 603 | WG containing repeat-containing protein | | CAETHG_0560 | CLJU_c24930 | CLRAG_17850 |
| 604 | integral membrane protein, YjbE family | | CAETHG_0561 | CLJU_c24940 | CLRAG_17840 |
| 605 | EDD domain protein, DegV family | | CAETHG_0563 | CLJU_c24960 | CLRAG_17820 |
| 606 | PucR C-terminal helix-turn-helix domain- | | CAETHG_0568 | CLJU_c25000 | CLRAG_17780 |
| | containing protein | | | | |
| 607 | hypothetical protein | | CAETHG_0570 | CLJU_c25020 | CLRAG_17760 |
| 608 | hypothetical protein | | CAETHG_0575 | CLJU_c25070 | CLRAG_17710 |
| 609 | ABC-2 family transporter protein | | CAETHG_0579 | CLJU_c25110 | CLRAG_03450 |
| 610 | hypothetical protein | | CAETHG_0582 | CLJU_c25140 | CLRAG_03480 |
| 611 | bla regulator protein blaR1 | | CAETHG_0585 | CLJU_c25170 | CLRAG_03510 |
| 612 | Putative amidase domain-containing protein | | CAETHG_0586 | CLJU_c25180 | CLRAG_03520 |
| 613 | D-alanyl-D-alanine carboxypeptidase | | CAETHG_0587 | CLJU_c25190 | CLRAG_03530 |
| 614 | hypothetical protein | | CAETHG_0596 | CLJU_c25270 | CLRAG_03620 |
| 615 | hypothetical protein | | CAETHG_0597 | CLJU_c25280 | CLRAG_03630 |
| 616 | Uncharacterized membrane protein YczE | | CAETHG_0621 | CLJU_c25520 | CLRAG_03730 |
| 617 | hypothetical protein | | CAETHG_0622 | CLJU_c25530 | CLRAG_03740 |
| 618 | PAP2 superfamily protein | | CAETHG_0625 | CLJU_c25550 | CLRAG_03750 |
| 619 | D-glucuronyl C5-epimerase C-terminus | | CAETHG_0628, | CLJU_c25590, | CLRAG_03790 |
| | | | CAETHG_0762 | CLJU_c26810 | |
| 620 | Alpha/beta hydrolase family protein | | CAETHG_0630 | CLJU_c25610 | CLRAG_03810 |
| 621 | ABC-2 family transporter protein | | CAETHG_0638 | CLJU_c25690 | CLRAG_03890 |
| 622 | ABC-2 family transporter protein | | CAETHG_0639 | CLJU_c25700 | CLRAG_03900 |
| 623 | hypothetical protein | | CAETHG_0643 | CLJU_c25740 | CLRAG_03970 |
| 624 | ABC-2 family transporter protein | | CAETHG_0657 | CLJU_c25880 | CLRAG_04020 |
| 625 | Putative zinc-finger | | CAETHG_0659 | CLJU_c25900 | CLRAG_04040 |
| 626 | Uncharacterized conserved protein, DUF2164 | | CAETHG_0661 | CLJU_c25920 | CLRAG_04060 |
| | family | | | | |
| 627 | Uncharacterized protein YjbI, contains | | CAETHG_0662 | CLJU_c25930 | CLRAG_04070 |
| | pentapeptide repeats | | | | |
| 628 | Protein of unknown function (DUF4003) | | CAETHG_0702 | CLJU_c26250 | CLRAG_04370 |
| 629 | hypothetical protein | | CAETHG_0714 | CLJU_c26330 | CLRAG_04420 |
| 630 | Conserved protein containing a Zn-ribbon-like | | CAETHG_0715 | CLJU_c26340 | CLRAG_04430 |
| | motif, possibly RNA-binding | | | | |
| 631 | Sulfite exporter TauE/SafE | | CAETHG_0723 | CLJU_c26420 | CLRAG_04510 |
| 632 | 4Fe—4S binding domain-containing protein | | CAETHG_0724 | CLJU_c26430 | CLRAG_04520 |
| 633 | hypothetical protein | | CAETHG_0725 | CLJU_c26440 | CLRAG_04530 |
| 634 | hypothetical protein | | CAETHG_0726 | CLJU_c26450 | CLRAG_04540 |
| 635 | hypothetical protein | | CAETHG_0731 | CLJU_c26500 | CLRAG_08380 |
| 636 | hypothetical protein | | CAETHG_0737 | CLJU_c26560 | CLRAG_08440 |
| 637 | spore photoproduct lyase | 4.1.99.— | CAETHG_0740 | CLJU_c26590 | CLRAG_08460 |
| 638 | Nucleoside phosphorylase | 3.2.2.16, | CAETHG_0741 | CLJU_c26600 | CLRAG_08470 |
| | | 3.2.2.9 | | | |
| 639 | Predicted nuclease of restriction endonuclease-like | | CAETHG_0744 | CLJU_c26630 | CLRAG_08500 |
| | (RecB) superfamily, DUF1016 family | | | | |
| 640 | 4Fe—4S dicluster domain-containing protein | | CAETHG_0749 | CLJU_c26680 | CLRAG_08550 |
| 641 | hypothetical protein | | CAETHG_0751 | CLJU_c26700 | CLRAG_08570 |
| 642 | uncharacterized protein | | CAETHG_0752 | CLJU_c26710 | CLRAG_08580 |
| 643 | membrane protein insertase, YidC/Oxa1 family, C- | | CAETHG_0756 | CLJU_c26750 | CLRAG_08620 |
| | terminal domain-containing protein | | | | |
| 644 | hypothetical protein | | CAETHG_0757 | CLJU_c26760 | CLRAG_08630 |
| 645 | hypothetical protein | | CAETHG_0761 | CLJU_c26800 | CLRAG_08670 |
| 646 | Acetyltransferase (GNAT) family protein | | CAETHG_0768 | CLJU_c26840 | CLRAG_08720 |
| 647 | Uncharacterized metal-binding protein | | CAETHG_0773 | CLJU_c37970, | CLRAG_37360 |
| | | | | CLJU_c26890 | |
| 648 | Uncharacterized protein family (UPF0051) | | CAETHG_0774, | CLJU_c37930, | CLRAG_37320 |
| | | | CAETHG_1630 | CLJU_c26900 | |
| 649 | Predicted permease | | CAETHG_0776 | CLJU_c26920 | CLRAG_08770 |
| 650 | Predicted permease | | CAETHG_0777 | CLJU_c26930 | CLRAG_08780 |
| 651 | ABC-2 family transporter protein | | CAETHG_0789 | CLJU_c27060 | CLRAG_08820 |
| 652 | Protoporphyrinogen IX oxidase, menaquinone- | | CAETHG_0795 | CLJU_c27100 | CLRAG_08880 |
| | dependent (flavodoxin domain) | | | | |
| 653 | hypothetical protein | | CAETHG_0800 | CLJU_c27140 | CLRAG_20090 |
| 654 | ABC-2 family transporter protein | | CAETHG_0801 | CLJU_c27150 | CLRAG_20080 |
| 655 | hypothetical protein | | CAETHG_0802, | CLJU_c00830, | CLRAG_20070 |
| | | | CAETHG_2198 | CLJU_c27160 | |
| 656 | hypothetical protein | | CAETHG_0811, | CLJU_c27250, | CLRAG_16540 |
| | | | CAETHG_3994 | CLJU_c18750 | |
| 657 | Spo0E like sporulation regulatory protein | | CAETHG_0824 | CLJU_c28270 | CLRAG_34350 |
| 658 | Serine phosphatase RsbU, regulator of sigma | | CAETHG_0827 | CLJU_c28300 | CLRAG_34380 |
| | subunit | | | | |
| 659 | protein of unknown function (DUF1987) | | CAETHG_0828 | CLJU_c28310 | CLRAG_34390 |
| 660 | hypothetical protein | | CAETHG_0829 | CLJU_c28320 | CLRAG_34400 |
| 661 | protein of unknown function (DUF4317) | | CAETHG_0830 | CLJU_c28330 | CLRAG_34410 |
| 662 | HesB-like selenoprotein | | CAETHG_0831 | CLJU_c28340 | CLRAG_34420 |
| 663 | IDEAL domain-containing protein | | CAETHG_0835 | CLJU_c28380 | CLRAG_34460 |
| 664 | PD-(D/E)XK nuclease superfamily protein | | CAETHG_0836 | CLJU_c28390 | CLRAG_34470 |
| 665 | hypothetical protein | | CAETHG_0839 | CLJU_c28410 | CLRAG_34490 |
| 666 | putative hemolysin | | CAETHG_0841 | CLJU_c28430 | CLRAG_34510 |
| 667 | Spo0E like sporulation regulatory protein | | CAETHG_0842 | CLJU_c28470 | CLRAG_34550 |
| 668 | hypothetical protein | | CAETHG_0846 | CLJU_c28510 | CLRAG_34590 |
| 669 | Uncharacterized membrane protein | | CAETHG_0850, | CLJU_c28560, | CLRAG_34630 |
| | | | CAETHG_4038 | CLJU_c19040 | |
| 670 | hypothetical protein | | CAETHG_0852 | CLJU_c28570 | CLRAG_34640 |
| 671 | hypothetical protein | | CAETHG_0857 | CLJU_c28620 | CLRAG_34700 |
| 672 | hypothetical protein | | CAETHG_0873 | CLJU_c28780 | CLRAG_34860 |
| 673 | Putative Flp pilus-assembly TadE/G-like | | CAETHG_0880 | CLJU_c28840 | CLRAG_34920 |
| 674 | pilus assembly protein Flp/PilA | | CAETHG_0882 | CLJU_c28860 | CLRAG_34940 |
| 675 | hypothetical protein | | CAETHG_0886 | CLJU_c28900 | CLRAG_34980 |
| 676 | Tryptophan transporter TrpP | | CAETHG_0892 | CLJU_c29000 | CLRAG_35000 |
| 677 | CBS domain-containing protein | 1.1.1.205 | CAETHG_0893 | CLJU_c29010 | CLRAG_35010 |
| 678 | hypothetical protein | | CAETHG_0894 | CLJU_c29020 | CLRAG_35020 |
| 679 | HNH endonuclease | | CAETHG_0895 | CLJU_c29030 | CLRAG_35030 |
| 680 | 40-residue YVTN family beta-propeller repeat- | | CAETHG_0900 | CLJU_c29080 | CLRAG_35080 |
| | containing protein | | | | |
| 681 | L,D-transpeptidase catalytic domain | | CAETHG_0901 | CLJU_c29090 | CLRAG_35090 |
| 682 | Uncharacterized conserved protein YukE | | CAETHG_0902 | CLJU_c29100 | CLRAG_35100 |
| 683 | hypothetical protein | | CAETHG_0911 | CLJU_c29190 | CLRAG_35190 |
| 684 | Uncharacterized conserved protein YgiM, contains | 3.5.1.28 | CAETHG_0912 | CLJU_c29200 | CLRAG_35200 |
| | N-terminal SH3 domain, DUF1202 family | | | | |
| 685 | hypothetical protein | | CAETHG_0918 | CLJU_c29250 | CLRAG_35260 |
| 686 | HD-GYP domain, c-di-GMP phosphodiesterase | | CAETHG_0919 | CLJU_c29260 | CLRAG_35270 |
| | class II (or its inactivated variant) | | | | |
| 687 | 2′-5′ RNA ligase | | CAETHG_0921 | CLJU_c29280 | CLRAG_35290 |
| 688 | hypothetical protein | | CAETHG_0922 | CLJU_c29290 | CLRAG_35300 |
| 689 | Protein of unknown function (DUF2000) | | CAETHG_0925 | CLJU_c29320 | CLRAG_35330 |
| 690 | protein of unknown function (DUF3787) | | CAETHG_0929 | CLJU_c29350 | CLRAG_35370 |
| 691 | SEC-C motif-containing protein | | CAETHG_0941 | CLJU_c29470 | CLRAG_35460 |
| 692 | hypothetical protein | | CAETHG_0942 | CLJU_c29480 | CLRAG_35470 |
| 693 | C_GCAxxG_C_C family probable redox protein | | CAETHG_0946 | CLJU_c29520 | CLRAG_35510 |
| 694 | hypothetical protein | | CAETHG_0965 | CLJU_c29670 | CLRAG_35670 |
| 695 | hypothetical protein | | CAETHG_0967 | CLJU_c29690 | CLRAG_35690 |
| 696 | hypothetical protein | | CAETHG_0973 | CLJU_c29740 | CLRAG_35740 |
| 697 | GDSL-like Lipase/Acylhydrolase | | CAETHG_0975 | CLJU_c29760 | CLRAG_35760 |
| 698 | Protein of unknown function (DUF3189) | | CAETHG_0976 | CLJU_c29770 | CLRAG_35770 |
| 699 | hypothetical protein | | CAETHG_0979 | CLJU_c29800 | CLRAG_35810 |
| 700 | hypothetical protein | | CAETHG_0981 | CLJU_c29820 | CLRAG_35830 |
| 701 | PrcB C-terminal | | CAETHG_0985 | CLJU_c29860 | CLRAG_35870 |
| 702 | protein of unknown function (DUF4367) | | CAETHG_0986 | CLJU_c29870 | CLRAG_35880 |
| 703 | Predicted transcriptional regulator YheO, contains | | CAETHG_1000 | CLJU_c30010 | CLRAG_15650 |
| | PAS and DNA-binding HTH domains | | | | |
| 704 | probable DNA metabolism protein | | CAETHG_1035, | CLJU_c30390, | CLRAG_15920 |
| | | | CAETHG_1044 | CLJU_c30280 | |
| 705 | YoaP-like | | CAETHG_1063 | CLJU_c30590 | CLRAG_16000 |
| 706 | hypothetical protein | | CAETHG_1075 | CLJU_c30710 | CLRAG_16150 |
| 707 | hypothetical protein | | CAETHG_1076 | CLJU_c30720 | CLRAG_16160 |
| 708 | hypothetical protein | | CAETHG_1083 | CLJU_c30790 | CLRAG_16230 |
| 709 | hypothetical protein | | CAETHG_1084 | CLJU_c30800 | CLRAG_16240 |
| 710 | Putative amidase domain-containing protein | | CAETHG_1091, | CLJU_c32160, | CLRAG_16290 |
| | | | CAETHG_1144 | CLJU_c30880 | |
| 711 | DNA-binding transcriptional regulator, FrmR family | | CAETHG_1142 | CLJU_c32140 | CLRAG_02770 |
| 712 | hypothetical protein | | CAETHG_1145 | CLJU_c32170 | CLRAG_02800 |
| 713 | Protein of unknown function (DUF3892) | | CAETHG_1148 | CLJU_c32200 | CLRAG_02830 |
| 714 | Peptidase MA superfamily | | CAETHG_1149 | CLJU_c32210 | CLRAG_02840 |
| 715 | Uncharacterized membrane protein | | CAETHG_1150 | CLJU_c32220 | CLRAG_03040 |
| 716 | YvrJ protein family protein | | CAETHG_1166, | CLJU_c32550, | CLRAG_37650 |
| | | | CAETHG_1657 | CLJU_c32360 | |
| 717 | Protein of unknown function (DUF1659) | | CAETHG_1167, | CLJU_c32560, | CLRAG_29820 |
| | | | CAETHG_1656 | CLJU_c32370 | |
| 718 | Protein of unknown function (DUF2922) | | CAETHG_1168, | CLJU_c32380, | CLRAG_29830 |
| | | | CAETHG_1655 | CLJU_c32570 | |
| 719 | protein of unknown function (DUF3786) | | CAETHG_1174, | CLJU_c32760, | CLRAG_06590 |
| | | | CAETHG_1517 | CLJU_c36080 | |
| 720 | Protein of unknown function (DUF2992) | | CAETHG_1180 | CLJU_c32820 | CLRAG_15510 |
| 721 | hypothetical protein | | CAETHG_1183 | CLJU_c32850 | CLRAG_15480 |
| 722 | hypothetical protein | | CAETHG_1184 | CLJU_c32860 | CLRAG_15470 |
| 723 | Phospholipid methyltransferase | | CAETHG_1188 | CLJU_c32900 | CLRAG_15430 |
| 724 | lia operon protein LiaG | | CAETHG_1189 | CLJU_c32910 | CLRAG_15420 |
| 725 | hypothetical protein | | CAETHG_1190 | CLJU_c32920 | CLRAG_15410 |
| 726 | Dolichyl-phosphate-mannose-protein | | CAETHG_1196, | CLJU_c32980, | CLRAG_15350 |
| | mannosyltransferase | | CAETHG_3415 | CLJU_c13320 | |
| 727 | hypothetical protein | | CAETHG_1197 | CLJU_c32990 | CLRAG_15340 |
| 728 | ABC-2 type transport system permease protein | | CAETHG_1198 | CLJU_c33000 | CLRAG_15330 |
| 729 | Haloacid Dehalogenase Superfamily Class | | CAETHG_1205 | CLJU_c33070 | CLRAG_15260 |
| | (subfamily) IIA/haloacid dehalogenase | | | | |
| | superfamily, subfamily IA, variant 1 with third | | | | |
| | motif having Dx(3-4)D or Dx(3-4)E | | | | |
| 730 | TIGR00659 family protein | | CAETHG_1206 | CLJU_c33080 | CLRAG_15250 |
| 731 | holin-like protein | | CAETHG_1207 | CLJU_c33090 | CLRAG_15240 |
| 732 | hypothetical protein | | CAETHG_1222 | CLJU_c33230 | CLRAG_15030 |
| 733 | hypothetical protein | | CAETHG_1228 | CLJU_c33290 | CLRAG_14970 |
| 734 | Uncharacterized membrane protein | | CAETHG_1231 | CLJU_c33320 | CLRAG_14940 |
| 735 | Uncharacterized spore protein YtfJ | | CAETHG_1234 | CLJU_c33340 | CLRAG_14910 |
| 736 | Protein of unknown function (DUF2953) | | CAETHG_1235 | CLJU_c33350 | CLRAG_14900 |
| 737 | hypothetical protein | | CAETHG_1236 | CLJU_c33360 | CLRAG_14890 |
| 738 | Lecithin retinol acyltransferase | | CAETHG_1237 | CLJU_c33370 | CLRAG_14880 |
| 739 | Spore coat protein CotF | | CAETHG_1239 | CLJU_c33390 | CLRAG_14860 |
| 740 | Spore coat associated protein JA (CotJA) | | CAETHG_1240 | CLJU_c33400 | CLRAG_14850 |
| 741 | hypothetical protein | | CAETHG_1244 | CLJU_c33440 | CLRAG_32320 |
| 742 | Protein of unknown function (DUF3100) | | CAETHG_1245 | CLJU_c33450 | CLRAG_32310 |
| 743 | hypothetical protein | | CAETHG_1254 | CLJU_c33550 | CLRAG_24730 |
| 744 | hypothetical protein | | CAETHG_1260 | CLJU_c33620 | CLRAG_24660 |
| 745 | MEDS: MEthanogen/methylotroph, DcmR Sensory | | CAETHG_1261 | CLJU_c33630 | CLRAG_24650 |
| | domain | | | | |
| 746 | hypothetical protein | | CAETHG_1263 | CLJU_c33650 | CLRAG_24630 |
| 747 | oxygen-independent coproporphyrinogen-3 | | CAETHG_1266 | CLJU_c33680 | CLRAG_24600 |
| | oxidase | | | | |
| 748 | six-cysteine peptide SCIFF | | CAETHG_1275 | CLJU_c33770 | CLRAG_24510 |
| 749 | putative membrane protein, TIGR04086 family | | CAETHG_1276 | CLJU_c33780 | CLRAG_24500 |
| 750 | preprotein translocase subunit YajC | | CAETHG_1277 | CLJU_c33790 | CLRAG_24490 |
| 751 | hypothetical protein | | CAETHG_1282 | CLJU_c33840 | CLRAG_24440 |
| 752 | UPF0755 protein | | CAETHG_1284 | CLJU_c33860 | CLRAG_24420 |
| 753 | DNA-binding regulatory protein, YebC/PmpR | | CAETHG_1285 | CLJU_c33870 | CLRAG_14020 |
| | family | | | | |
| 754 | uncharacterized protein, YigZ family | | CAETHG_1286 | CLJU_c33880 | CLRAG_14030 |
| 755 | hypothetical protein | | CAETHG_1288 | CLJU_c33900 | CLRAG_14050 |
| 756 | sporulation protein YunB | | CAETHG_1291 | CLJU_c33930 | CLRAG_14080 |
| 757 | O-antigen ligase | | CAETHG_1304 | CLJU_c34060 | CLRAG_14210 |
| 758 | oligosaccharide repeat unit polymerase | | CAETHG_1312 | CLJU_c34140 | CLRAG_14290 |
| 759 | hypothetical protein | | CAETHG_1324, | CLJU_c34320, | CLRAG_14380 |
| | | | CAETHG_1332 | CLJU_c34250 | |
| 760 | Uncharacterized conserved protein YbbK, DUF523 | | CAETHG_1337 | CLJU_c34370 | CLRAG_14450 |
| | family | | | | |
| 761 | cell division protein ZapA | | CAETHG_1340 | CLJU_c34400 | CLRAG_14480 |
| 762 | putative sporulation protein YtxC | | CAETHG_1348 | CLJU_c34480 | CLRAG_14560 |
| 763 | Putative peptidoglycan binding domain-containing | | CAETHG_1349 | CLJU_c34530 | CLRAG_14610 |
| | protein | | | | |
| 764 | hypothetical protein | | CAETHG_1359 | CLJU_c34630 | CLRAG_14710 |
| 765 | protein of unknown function (DUF4364) | | CAETHG_1361 | CLJU_c34650 | CLRAG_14730 |
| 766 | 40-residue YVTN family beta-propeller repeat- | | CAETHG_1363 | CLJU_c34660 | CLRAG_14740 |
| | containing protein | | | | |
| 767 | uncharacterized protein TIGR03905 | | CAETHG_1364 | CLJU_c34670 | CLRAG_14750 |
| 768 | hypothetical protein | | CAETHG_1367 | CLJU_c34700 | CLRAG_14780 |
| 769 | NitT/TauT family transport system substrate- | | CAETHG_1368 | CLJU_c34710 | CLRAG_14790 |
| | binding protein | | | | |
| 770 | Protein of unknown function (DUF2812) | | CAETHG_1381 | CLJU_c34830 | CLRAG_26070 |
| 771 | protein CcmA, bactofilin family | | CAETHG_1389 | CLJU_c34910 | CLRAG_26150 |
| 772 | Protein of unknown function (DUF4004) | | CAETHG_1390 | CLJU_c34920 | CLRAG_26160 |
| 773 | Polyketide cyclase/dehydrase | | CAETHG_1407 | CLJU_c34980 | CLRAG_26250 |
| 774 | AAA domain-containing protein | | CAETHG_1408 | CLJU_c34990 | CLRAG_26260 |
| 775 | energy coupling factor transporter S component | | CAETHG_1414 | CLJU_c35050 | CLRAG_26310 |
| | ThiW | | | | |
| 776 | DNA-binding transcriptional regulator, PadR family | | CAETHG_1421 | CLJU_c35120 | CLRAG_26370 |
| 777 | ABC-2 type transport system permease protein | | CAETHG_1428 | CLJU_c35200 | CLRAG_05710 |
| 778 | ABC-2 type transport system permease protein | | CAETHG_1429 | CLJU_c35210 | CLRAG_05720 |
| 779 | Uncharacterized conserved protein YurZ, | | CAETHG_1430 | CLJU_c35220 | CLRAG_05730 |
| | alkylhydroperoxidase/carboxymuconolactone | | | | |
| | decarboxylase family | | | | |
| 780 | putative membrane protein | | CAETHG_1433 | CLJU_c35250 | CLRAG_05760 |
| 781 | NitT/TauT family transport system substrate- | | CAETHG_1441 | CLJU_c35320 | CLRAG_05850 |
| | binding protein | | | | |
| 782 | Tetratricopeptide repeat-containing protein | | CAETHG_1446 | CLJU_c35380 | CLRAG_05920 |
| 783 | Uncharacterized membrane-anchored protein YitT, | | CAETHG_1454 | CLJU_c35460 | CLRAG_05990 |
| | contains DUF161 and DUF2179 domains | | | | |
| 784 | hybrid cluster protein-associated redox disulfide | | CAETHG_1457, | CLJU_c35500, | CLRAG_06020 |
| | domain-containing protein | | CAETHG_3809, | CLJU_c35490, | |
| | | | CAETHG_1458 | CLJU_c16990 | |
| 785 | Repeat domain-containing protein | | CAETHG_1465 | CLJU_c35570 | CLRAG_06100 |
| 786 | hypothetical protein | | CAETHG_1467 | CLJU_c35590 | CLRAG_06120 |
| 787 | CAAX protease self-immunity | | CAETHG_1468 | CLJU_c35600 | CLRAG_06130 |
| 788 | hypothetical protein | | CAETHG_1475 | CLJU_c35670 | CLRAG_06200 |
| 789 | Protein of unknown function (DUF3343) | | CAETHG_1483 | CLJU_c35750 | CLRAG_06280 |
| 790 | Uncharacterized membrane-anchored protein YitT, | | CAETHG_1487 | CLJU_c35790 | CLRAG_06320 |
| | contains DUF161 and DUF2179 domains | | | | |
| 791 | Probable zinc-ribbon domain-containing protein | | CAETHG_1488, | CLJU_c38240, | CLRAG_06330 |
| | | | CAETHG_1680 | CLJU_c35800 | |
| 792 | dihydropyrimidine dehydrogenase (NAD+) subunit | | CAETHG_1495 | CLJU_c35880 | CLRAG_06380 |
| | PreT | | | | |
| 793 | hypothetical protein | | CAETHG_1505 | CLJU_c35970 | CLRAG_06470 |
| 794 | hypothetical protein | | CAETHG_1515 | CLJU_c36060 | CLRAG_06550 |
| 795 | YvrJ protein family protein | | CAETHG_1516 | CLJU_c36070 | CLRAG_06560 |
| 796 | hypothetical protein | | CAETHG_1522 | CLJU_c36120 | CLRAG_06650 |
| 797 | hypothetical protein | | CAETHG_1528 | CLJU_c36180 | CLRAG_24000 |
| 798 | hypothetical protein | | CAETHG_1529 | CLJU_c36190 | CLRAG_23990 |
| 799 | hypothetical protein | | CAETHG_1540 | CLJU_c36320 | CLRAG_23840 |
| 800 | hypothetical protein | | CAETHG_1549 | CLJU_c36880 | CLRAG_36410 |
| 801 | hypothetical protein | | CAETHG_1551 | CLJU_c36900 | CLRAG_36430 |
| 802 | hypothetical protein | | CAETHG_1557 | CLJU_c36960 | CLRAG_36500 |
| 803 | hypothetical protein | | CAETHG_1564 | CLJU_c37090 | CLRAG_36570 |
| 804 | hypothetical protein | | CAETHG_1565 | CLJU_c37100 | CLRAG_36580 |
| 805 | Uncharacterized membrane protein YdjX, | | CAETHG_1574 | CLJU_c37210 | CLRAG_36650 |
| | TVP38/TMEM64 family, SNARE-associated domain | | | | |
| 806 | uncharacterized protein, MTH1187 family | | CAETHG_1587 | CLJU_c37310 | CLRAG_36750 |
| 807 | protein of unknown function (DUF1836) | | CAETHG_1597 | CLJU_c37450 | CLRAG_36860 |
| 808 | CxxC motif-containing protein | | CAETHG_1598 | CLJU_c37460 | CLRAG_36870 |
| 809 | protein of unknown function (DUF3786) | | CAETHG_1603 | CLJU_c37500 | CLRAG_36910 |
| 810 | hypothetical protein | | CAETHG_1604 | CLJU_c22610, | CLRAG_36920 |
| | | | | CLJU_c37510 | |
| 811 | Predicted RNA-binding protein | | CAETHG_1605 | CLJU_c37520 | CLRAG_36930 |
| 812 | Methylene-tetrahydrofolate reductase C terminal | 1.5.1.20 | CAETHG_1615 | CLJU_c37620 | CLRAG_37030 |
| 813 | hypothetical protein | | CAETHG_1626 | CLJU_c37790 | CLRAG_37210 |
| 814 | conserved hypothetical integral membrane protein | | CAETHG_1628 | CLJU_c37810 | CLRAG_37230 |
| 815 | MOSC domain containing protein | | CAETHG_1632 | CLJU_c38010 | CLRAG_37380 |
| 816 | hypothetical protein | | CAETHG_1635 | CLJU_c38110 | CLRAG_37480 |
| 817 | hypothetical protein | | CAETHG_1639 | CLJU_c38130 | CLRAG_37530 |
| 818 | Protein of unknown function (DUF1653) | | CAETHG_1644, | CLJU_c08310 | CLRAG_08090 |
| | | | CAETHG_2926 | | |
| 819 | DnaD and phage-associated domain-containing | | CAETHG_1665 | CLJU_c32520 | CLRAG_36090 |
| | protein | | | | |
| 820 | hypothetical protein | | CAETHG_1678 | CLJU_c38220 | CLRAG_20680 |
| 821 | Predicted transcriptional regulator, contains HTH | | CAETHG_1679 | CLJU_c38230 | CLRAG_20690 |
| | domain | | | | |
| 822 | Uncharacterized membrane protein | | CAETHG_1681 | CLJU_c38250 | CLRAG_20710 |
| 823 | GxGYxY sequence motif-containing protein | | CAETHG_1695 | CLJU_c38370 | CLRAG_20840 |
| 824 | hypothetical protein | | CAETHG_1699 | CLJU_c38440 | CLRAG_20880 |
| 825 | hypothetical protein | | CAETHG_1751 | CLJU_c31340 | CLRAG_21210 |
| 826 | preprotein translocase subunit SecG | | CAETHG_1755 | CLJU_c39100 | CLRAG_21250 |
| 827 | hypothetical protein | | CAETHG_1766 | CLJU_c39210 | CLRAG_21360 |
| 828 | Threonine/homoserine efflux transporter RhtA | | CAETHG_1767 | CLJU_c39220 | CLRAG_21430 |
| 829 | Ig-like domain-containing protein | | CAETHG_1778 | CLJU_c39330 | CLRAG_21540 |
| 830 | hypothetical protein | | CAETHG_1783 | CLJU_c39380 | CLRAG_21590 |
| 831 | hypothetical protein | | CAETHG_1792 | CLJU_c39470 | CLRAG_21680 |
| 832 | hypothetical protein | | CAETHG_1793 | CLJU_c39480 | CLRAG_21690 |
| 833 | hypothetical protein | | CAETHG_1794 | CLJU_c39490 | CLRAG_21700 |
| 834 | hypothetical protein | | CAETHG_1798 | CLJU_c39530 | CLRAG_21730 |
| 835 | homoserine kinase | | CAETHG_1811 | CLJU_c39650 | CLRAG_21900 |
| 836 | Transcriptional regulator PadR-like family protein | | CAETHG_1833, | CLJU_c39890, | CLRAG_22120 |
| | | | CAETHG_1835 | CLJU_c39870 | |
| 837 | Multidrug resistance efflux transporter | | CAETHG_1834, | CLJU_c39900, | CLRAG_22130 |
| | | | CAETHG_1836 | CLJU_c39880 | |
| 838 | membrane protein DedA, SNARE-associated | | CAETHG_1842 | CLJU_c39960 | CLRAG_22190 |
| | domain | | | | |
| 839 | Putative amidase domain-containing protein | | CAETHG_1849 | CLJU_c40020 | CLRAG_22250 |
| 840 | Mor transcription activator family protein | | CAETHG_1856 | CLJU_c40070 | CLRAG_22330 |
| 841 | phosphopantothenoylcysteine decarboxylase/ | 4.1.1.36, | CAETHG_1863 | CLJU_c40140 | CLRAG_22370 |
| | phosphopantothenate--cysteine ligase | 6.3.2.5 | | | |
| 842 | protein of unknown function (DUF4177) | | CAETHG_1886 | CLJU_c40430 | CLRAG_22620 |
| 843 | YbbR domain-containing protein | | CAETHG_1889 | CLJU_c40460 | CLRAG_22650 |
| 844 | diadenylate cyclase | | CAETHG_1890 | CLJU_c40470 | CLRAG_22660 |
| 845 | protein of unknown function (DUF4652) | | CAETHG_1895 | CLJU_c40520 | CLRAG_22710 |
| 846 | ribonuclease-3 family protein | | CAETHG_1967 | CLJU_c41260 | CLRAG_23450 |
| 847 | Uncharacterized conserved protein YacL, contains | | CAETHG_1970 | CLJU_c41290 | CLRAG_23480 |
| | PIN and TRAM domains | | | | |
| 848 | hypothetical protein | | CAETHG_1971 | CLJU_c41300 | CLRAG_23490 |
| 849 | hypothetical protein | | CAETHG_1978 | CLJU_c41370 | CLRAG_23560 |
| 850 | hypothetical protein | 2.6.1.11 | CAETHG_1984 | CLJU_c41500 | CLRAG_04630 |
| 851 | cell division protein FtsL | | CAETHG_1992 | CLJU_c41630 | CLRAG_04760 |
| 852 | spore cortex biosynthesis protein YabQ | | CAETHG_1993 | CLJU_c41640 | CLRAG_04770 |
| 853 | sporulation protein YabP | | CAETHG_1994 | CLJU_c41650 | CLRAG_04780 |
| 854 | tetrapyrrole methylase family protein/MazG | | CAETHG_1997 | CLJU_c41680 | CLRAG_04810 |
| | family protein | | | | |
| 855 | hypothetical protein | | CAETHG_2011 | CLJU_c41820 | CLRAG_04950 |
| 856 | hypothetical protein | | CAETHG_2016 | CLJU_c41870 | CLRAG_04980 |
| 857 | hypothetical protein | | CAETHG_2019 | CLJU_c41900 | CLRAG_05010 |
| 858 | Uncharacterized membrane protein YkvI | | CAETHG_2020 | CLJU_c41910 | CLRAG_05020 |
| 859 | 2′-5′ RNA ligase superfamily protein | | CAETHG_2025 | CLJU_c41960 | CLRAG_05070 |
| 860 | protein of unknown function (DUF1540) | | CAETHG_2026 | CLJU_c41970 | CLRAG_05080 |
| 861 | CxxH/CxxC protein, BA_5709 family | | CAETHG_2029 | CLJU_c42000 | CLRAG_05110 |
| 862 | SEC-C motif-containing protein | | CAETHG_2031 | CLJU_c42010 | CLRAG_05120 |
| 863 | F5/8 type C domain-containing protein | | CAETHG_2032 | CLJU_c42020 | CLRAG_05130 |
| 864 | putative iron-only hydrogenase system regulator | | CAETHG_2034 | CLJU_c42040 | CLRAG_05150 |
| 865 | Dimeric dUTPase, all-alpha-NTP-PPase (MazG) | | CAETHG_2035 | CLJU_c42050 | CLRAG_05160 |
| | superfamily | | | | |
| 866 | transporter family-2 protein | | CAETHG_2036 | CLJU_c42060 | CLRAG_05170 |
| 867 | NAD(P)H-dependent flavin oxidoreductase YrpB, | | CAETHG_2052 | CLJU_c42210 | CLRAG_05320 |
| | nitropropane dioxygenase family | | | | |
| 868 | Predicted CoA-binding protein | | CAETHG_2056 | CLJU_c42320 | CLRAG_05430 |
| 869 | Cupin domain-containing protein | | CAETHG_2068 | CLJU_c42430 | CLRAG_05570 |
| 870 | Putative ABC-transporter type IV | | CAETHG_2072 | CLJU_c42470 | CLRAG_05580 |
| 871 | MazG-like family protein | | CAETHG_2102 | CLJU_c42760 | CLRAG_25570 |
| 872 | hypothetical protein | | CAETHG_2106 | CLJU_c42800 | CLRAG_25610 |
| 873 | Protein of unknown function (DUF3343) | | CAETHG_2108 | CLJU_c42820 | CLRAG_25630 |
| 874 | sporulation integral membrane protein YtvI | | CAETHG_2109 | CLJU_c42830 | CLRAG_25640 |
| 875 | putative sporulation protein YyaC | | CAETHG_2110 | CLJU_c42840 | CLRAG_25650 |
| 876 | Uncharacterised protein family (UPF0180) | | CAETHG_2111 | CLJU_c42850 | CLRAG_25660 |
| 877 | Protein of unknown function (DUF4446) | | CAETHG_2112 | CLJU_c42860 | CLRAG_25670 |
| 878 | hypothetical protein | | CAETHG_2121 | CLJU_c42950 | CLRAG_25760 |
| 879 | ribosome-associated protein | | CAETHG_2126 | CLJU_c00030 | CLRAG_25810 |
| 880 | protein of unknown function (DUF370) | | CAETHG_2128 | CLJU_c00050 | CLRAG_25830 |
| 881 | HDIG domain-containing protein | | CAETHG_2131 | CLJU_c00120 | CLRAG_20200 |
| 882 | hypothetical protein | | CAETHG_2133 | CLJU_c00140 | CLRAG_20180 |
| 883 | Protein of unknown function (DUF1667) | | CAETHG_2136 | CLJU_c00160 | CLRAG_20160 |
| 884 | hypothetical protein | | CAETHG_2173 | CLJU_c00550 | CLRAG_20020 |
| 885 | Mannose-6-phosphate isomerase, cupin | | CAETHG_2181 | CLJU_c00630 | CLRAG_19950 |
| | superfamily | | | | |
| 886 | Polyhydroxyalkanoate synthesis regulator phasin | | CAETHG_2191 | CLJU_c00740 | CLRAG_19840 |
| 887 | hypothetical protein | | CAETHG_2200 | CLJU_c00860 | CLRAG_19720 |
| 888 | Protein of unknown function (DUF2508) | | CAETHG_2202 | CLJU_c00880 | CLRAG_19700 |
| 889 | inhibitor of the pro-sigma K processing machinery | | CAETHG_2203 | CLJU_c00890 | CLRAG_19690 |
| 890 | hypothetical protein | | CAETHG_2206, | CLJU_c06660, | CLRAG_19660 |
| | | | CAETHG_2756 | CLJU_c00920 | |
| 891 | 4Fe—4S dicluster domain-containing protein | | CAETHG_2207 | CLJU_c00930 | CLRAG_19650 |
| 892 | hypothetical protein | | CAETHG_2208 | CLJU_c00940 | CLRAG_19640 |
| 893 | hypothetical protein | | CAETHG_2209 | CLJU_c00950 | CLRAG_19630 |
| 894 | hypothetical protein | | CAETHG_2240 | CLJU_c01340 | CLRAG_27000 |
| 895 | Inhibitor of sigma-G Gin | | CAETHG_2243 | CLJU_c01370 | CLRAG_27030 |
| 896 | Uncharacterized protein YaaQ | | CAETHG_2246 | CLJU_c01400 | CLRAG_27060 |
| 897 | Copper chaperone CopZ | | CAETHG_2249 | CLJU_c01430 | CLRAG_27090 |
| 898 | Cell wall-associated hydrolase, NlpC family | | CAETHG_2255, | CLJU_c01500, | CLRAG_27150 |
| | | | CAETHG_2256 | CLJU_c01490 | |
| 899 | hypothetical protein | | CAETHG_2259 | CLJU_c01530 | CLRAG_27190 |
| 900 | protein of unknown function (DUF1836) | | CAETHG_2262 | CLJU_c01560 | CLRAG_27220 |
| 901 | hypothetical protein | | CAETHG_2264 | CLJU_c01580 | CLRAG_27240 |
| 902 | hypothetical protein | | CAETHG_2270 | CLJU_c01680 | CLRAG_27280 |
| 903 | hypothetical protein | | CAETHG_2271 | CLJU_c01690 | CLRAG_27290 |
| 904 | protein of unknown function (DUF348) | | CAETHG_2277 | CLJU_c01750 | CLRAG_27350 |
| 905 | ribonuclease M5 | | CAETHG_2278 | CLJU_c01760 | CLRAG_27360 |
| 906 | hypothetical protein | | CAETHG_2281 | CLJU_c01780 | CLRAG_27380 |
| 907 | hypothetical protein | | CAETHG_2282 | CLJU_c01790 | CLRAG_27390 |
| 908 | hypothetical protein | | CAETHG_2283 | CLJU_c01800 | CLRAG_27400 |
| 909 | hypothetical protein | | CAETHG_2286 | CLJU_c01830 | CLRAG_27430 |
| 910 | membrane protein DedA, SNARE-associated | | CAETHG_2294 | CLJU_c01910 | CLRAG_27510 |
| | domain | | | | |
| 911 | Part of AAA domain-containing protein | | CAETHG_2295 | CLJU_c01920 | CLRAG_27520 |
| 912 | putative endonuclease | | CAETHG_2297 | CLJU_c01940 | CLRAG_27540 |
| 913 | hypothetical protein | | CAETHG_2299 | CLJU_c01960 | CLRAG_27560 |
| 914 | Uncharacterized protein Veg | | CAETHG_2312 | CLJU_c02070 | CLRAG_27670 |
| 915 | protein of unknown function (DUF3794) | | CAETHG_2313 | CLJU_c02080 | CLRAG_27680 |
| 916 | hypothetical protein | | CAETHG_2317 | CLJU_c02120 | CLRAG_27720 |
| 917 | Uncharacterized beta-barrel protein YwiB, | | CAETHG_2323 | CLJU_c02180 | CLRAG_27780 |
| | DUF1934 family | | | | |
| 918 | hypothetical protein | | CAETHG_2326 | CLJU_c02210 | CLRAG_27810 |
| 919 | Uncharacterized conserved protein YqhQ | | CAETHG_2329 | CLJU_c02240 | CLRAG_27840 |
| 920 | hypothetical protein | | CAETHG_2332 | CLJU_c02270 | CLRAG_27870 |
| 921 | TATA-box binding | | CAETHG_2351 | CLJU_c02450 | CLRAG_28060 |
| 922 | putative sporulation protein YyaC | | CAETHG_2357 | CLJU_c02520 | CLRAG_28120 |
| 923 | hypothetical protein | | CAETHG_2360 | CLJU_c02600 | CLRAG_28200 |
| 924 | Riboflavin transporter FmnP | | CAETHG_2370 | CLJU_c02690 | CLRAG_28290 |
| 925 | hypothetical protein | | CAETHG_2372 | CLJU_c02700 | CLRAG_28300 |
| 926 | flagellar biosynthesis protein | | CAETHG_2373 | CLJU_c02710 | CLRAG_28310 |
| 927 | hypothetical protein | | CAETHG_2378 | CLJU_c02770 | CLRAG_28370 |
| 928 | DnaJ domain-containing protein | | CAETHG_2379 | CLJU_c02780 | CLRAG_28380 |
| 929 | hypothetical protein | | CAETHG_2380 | CLJU_c02790 | CLRAG_28390 |
| 930 | protein of unknown function (DUF4363) | | CAETHG_2383 | CLJU_c02820 | CLRAG_28420 |
| 931 | Uncharacterized membrane protein YcaP, DUF421 | | CAETHG_2384 | CLJU_c02830 | CLRAG_28430 |
| | family | | | | |
| 932 | uridine kinase | 2.7.1.48 | CAETHG_2385 | CLJU_c02840 | CLRAG_28440 |
| 933 | serine/threonine-protein kinase RsbW | | CAETHG_2403 | CLJU_c02880 | CLRAG_28480 |
| 934 | hypothetical protein | | CAETHG_2405 | CLJU_c02900 | CLRAG_28500 |
| 935 | Uncharacterized membrane protein YdjX, | | CAETHG_2407 | CLJU_c02920 | CLRAG_28520 |
| | TVP38/TMEM64 family, SNARE-associated domain | | | | |
| 936 | CBS domain-containing protein | 1.1.1.205 | CAETHG_2408 | CLJU_c02930 | CLRAG_28530 |
| 937 | hypothetical protein | | CAETHG_2410 | CLJU_c02950 | CLRAG_28560 |
| 938 | hypothetical protein | | CAETHG_2414 | CLJU_c02990 | CLRAG_28600 |
| 939 | Outer membrane lipoprotein-sorting protein | | CAETHG_2417 | CLJU_c03020 | CLRAG_28630 |
| 940 | PDZ domain-containing protein | | CAETHG_2425 | CLJU_c03100 | CLRAG_28710 |
| 941 | Forkhead associated (FHA) domain, binds pSer, | | CAETHG_2428 | CLJU_c03130 | CLRAG_28740 |
| | pThr, pTyr | | | | |
| 942 | inner membrane protein | | CAETHG_2431 | CLJU_c03160 | CLRAG_28770 |
| 943 | UPF0042 nucleotide-binding protein | | CAETHG_2434 | CLJU_c03190 | CLRAG_28800 |
| 944 | conserved hypothetical protein, cofD-related | | CAETHG_2435 | CLJU_c03200 | CLRAG_28810 |
| 945 | hypothetical protein | | CAETHG_2436 | CLJU_c03210 | CLRAG_28820 |
| 946 | Uncharacterized conserved protein YgbK, DUF1537 | | CAETHG_2444 | CLJU_c03820 | CLRAG_28890 |
| | family | | | | |
| 947 | Uncharacterised protein family (UPF0180) | | CAETHG_2451 | CLJU_c03890 | CLRAG_29000 |
| 948 | protein of unknown function (DUF3870) | | CAETHG_2455 | CLJU_c03930 | CLRAG_29040 |
| 949 | EamA domain-containing membrane protein RarD | | CAETHG_2456 | CLJU_c03940 | CLRAG_29050 |
| 950 | Methyltransferase domain-containing protein | | CAETHG_2473 | CLJU_c04100 | CLRAG_26950 |
| 951 | hypothetical protein | | CAETHG_2489 | CLJU_c04210 | CLRAG_26850 |
| 952 | hypothetical protein | | CAETHG_2490 | CLJU_c04220 | CLRAG_26840 |
| 953 | hypothetical protein | | CAETHG_2491 | CLJU_c04230 | CLRAG_26830 |
| 954 | Uncharacterized protein, contains a NRPS | | CAETHG_2492 | CLJU_c04240 | CLRAG_26790 |
| | condensation (elongation) domain | | | | |
| 955 | hypothetical protein | | CAETHG_2500 | CLJU_c04320 | CLRAG_26730 |
| 956 | Putative flippase GtrA (transmembrane | | CAETHG_2518 | CLJU_c04460 | CLRAG_37820 |
| | translocase of bactoprenol-linked glucose) | | | | |
| 957 | Protein of unknown function (DUF2837) | | CAETHG_2522 | CLJU_c04500 | CLRAG_37860 |
| 958 | GPI transamidase subunit PIG-U | | CAETHG_2524 | CLJU_c04520 | CLRAG_37880 |
| 959 | TGF-beta propeptide | | CAETHG_2525 | CLJU_c04530 | CLRAG_37890 |
| 960 | ABC-2 type transport system permease protein | | CAETHG_2529 | CLJU_c04570 | CLRAG_37930 |
| 961 | Uncharacterized membrane protein | | CAETHG_2536 | CLJU_c04640 | CLRAG_38000 |
| 962 | Protein of unknown function DUF116 | | CAETHG_2539 | CLJU_c04670 | CLRAG_38050 |
| 963 | hypothetical protein | | CAETHG_2542 | CLJU_c04700 | CLRAG_38080 |
| 964 | hypothetical protein | | CAETHG_2543 | CLJU_c04710 | CLRAG_38090 |
| 965 | hypothetical protein | | CAETHG_2544 | CLJU_c04720 | CLRAG_38100 |
| 966 | hypothetical protein | | CAETHG_2552 | CLJU_c04800 | CLRAG_38180 |
| 967 | hypothetical protein | | CAETHG_2553 | CLJU_c04810 | CLRAG_38190 |
| 968 | Putative cell wall binding repeat 2 | | CAETHG_2555 | CLJU_c04830 | CLRAG_38210 |
| 969 | spore coat assembly protein SafA/uncharacterized | | CAETHG_2563 | CLJU_c04860 | CLRAG_38250 |
| | protein, YkwD family | | | | |
| 970 | Outer membrane protein TolC | | CAETHG_2583 | CLJU_c05060 | CLRAG_38450 |
| 971 | Outer membrane efflux protein | | CAETHG_2584 | CLJU_c05070 | CLRAG_38460 |
| 972 | ResB family protein | | CAETHG_2585 | CLJU_c05080 | CLRAG_38470 |
| 973 | WG containing repeat-containing protein | | CAETHG_2586 | CLJU_c05090 | CLRAG_38480 |
| 974 | SLAP domain-containing protein | | CAETHG_2587 | CLJU_c05100 | CLRAG_38490 |
| 975 | hypothetical protein (DUF2140) | | CAETHG_2590 | CLJU_c05130 | CLRAG_38520 |
| 976 | Protein of unknown function (DUF1659) | | CAETHG_2594 | CLJU_c05170 | CLRAG_38560 |
| 977 | Protein of unknown function (DUF2922) | | CAETHG_2595 | CLJU_c05180 | CLRAG_38570 |
| 978 | RNA polymerase sporulation-specific sigma factor | | CAETHG_2596 | CLJU_c05190 | CLRAG_38580 |
| 979 | O-antigen polymerase | | CAETHG_2626 | CLJU_c05440 | CLRAG_38900 |
| 980 | Acetyltransferase (isoleucine patch superfamily) | | CAETHG_2627 | CLJU_c05450 | CLRAG_38910 |
| 981 | asparagine synthase (glutamine-hydrolysing) | 6.3.5.4 | CAETHG_2628 | CLJU_c05460 | CLRAG_38920 |
| 982 | Capsule polysaccharide biosynthesis protein | | CAETHG_2629 | CLJU_c05470 | CLRAG_38930 |
| 983 | putative Mg2+ transporter-C (MgtC) family protein | | CAETHG_2654 | CLJU_c05620 | CLRAG_06840 |
| 984 | hypothetical protein | | CAETHG_2659 | CLJU_c05670 | CLRAG_06900 |
| 985 | hypothetical protein | | CAETHG_2660 | CLJU_c05680 | CLRAG_06910 |
| 986 | hypothetical protein | | CAETHG_2661 | CLJU_c05690 | CLRAG_06920 |
| 987 | von Willebrand factor type A domain-containing | | CAETHG_2662 | CLJU_c05700 | CLRAG_06930 |
| | protein | | | | |
| 988 | Tubulin like | | CAETHG_2663 | CLJU_c05710 | CLRAG_06940 |
| 989 | hypothetical protein | | CAETHG_2664 | CLJU_c05720 | CLRAG_06950 |
| 990 | Ca-activated chloride channel family protein | | CAETHG_2666 | CLJU_c05730 | CLRAG_06960 |
| 991 | hypothetical protein | | CAETHG_2667 | CLJU_c05740 | CLRAG_06970 |
| 992 | GTP1/OBG protein | | CAETHG_2673 | CLJU_c05780 | CLRAG_07030 |
| 993 | hypothetical protein | | CAETHG_2674 | CLJU_c05790 | CLRAG_07040 |
| 994 | zinc-ribbon domain-containing protein | | CAETHG_2675 | CLJU_c05800 | CLRAG_07050 |
| 995 | Protein of unknown function (DUF1861) | | CAETHG_2682 | CLJU_c05870 | CLRAG_07110 |
| 996 | VanZ like family protein | | CAETHG_2690 | CLJU_c05930 | CLRAG_07160 |
| 997 | hypothetical protein | | CAETHG_2699 | CLJU_c06010 | CLRAG_07240 |
| 998 | Putative Mn2+ efflux pump MntP | | CAETHG_2705 | CLJU_c06060 | CLRAG_07330 |
| 999 | hypothetical protein | | CAETHG_2707 | CLJU_c06080 | CLRAG_07350 |
| 1000 | Uncharacterized membrane protein YkvA, | | CAETHG_2711 | CLJU_c06120 | CLRAG_07400 |
| | DUF1232 family | | | | |
| 1001 | hypothetical protein | | CAETHG_2713 | CLJU_c06140 | CLRAG_07420 |
| 1002 | Peptidase family S41 | | CAETHG_2742 | CLJU_c06460 | CLRAG_30440 |
| 1003 | hypothetical protein | | CAETHG_2763 | CLJU_c06720 | CLRAG_18520 |
| 1004 | hypothetical protein | | CAETHG_2766 | CLJU_c06750 | CLRAG_18550 |
| 1005 | hypothetical protein | | CAETHG_2771 | CLJU_c06800 | CLRAG_18600 |
| 1006 | Carbonic anhydrase or acetyltransferase, | | CAETHG_2776 | CLJU_c06850 | CLRAG_18690 |
| | isoleucine patch superfamily | | | | |
| 1007 | hypothetical protein | | CAETHG_2778 | CLJU_c06870 | CLRAG_18710 |
| 1008 | Peptidoglycan/LPS O-acetylase OafA/YrhL, | | CAETHG_2781 | CLJU_c06910 | CLRAG_18750 |
| | contains acyltransferase and SGNH-hydrolase | | | | |
| | domains | | | | |
| 1009 | Membrane protein implicated in regulation of | | CAETHG_2783 | CLJU_c06930 | CLRAG_18770 |
| | membrane protease activity | | | | |
| 1010 | hypothetical protein | | CAETHG_2787 | CLJU_c06970 | CLRAG_18810 |
| 1011 | Acetyltransferase (GNAT) domain-containing | | CAETHG_2809 | CLJU_c07170 | CLRAG_26670 |
| | protein | | | | |
| 1012 | protein of unknown function (DUF4321) | | CAETHG_2811 | CLJU_c07190 | CLRAG_26650 |
| 1013 | radical SAM-linked protein | | CAETHG_2826 | CLJU_c07340 | CLRAG_26500 |
| 1014 | hypothetical protein | | CAETHG_2829 | CLJU_c07370 | CLRAG_26470 |
| 1015 | 2-iminobutanoate/2-iminopropanoate deaminase | | CAETHG_2834 | CLJU_c07420 | CLRAG_26420 |
| 1016 | putative redox protein | | CAETHG_2854 | CLJU_c07610 | CLRAG_25180 |
| 1017 | Ribosomal protein L7/L12 C-terminal domain- | | CAETHG_2876 | CLJU_c07830 | CLRAG_25300 |
| | containing protein | | | | |
| 1018 | protein of unknown function DUF4412 | | CAETHG_2878 | CLJU_c07850 | CLRAG_25330 |
| 1019 | hypothetical protein | | CAETHG_2879 | CLJU_c07860 | CLRAG_25340 |
| 1020 | competence protein ComEC | | CAETHG_2881 | CLJU_c07880 | CLRAG_25360 |
| 1021 | hypothetical protein | | CAETHG_2886 | CLJU_c07930 | CLRAG_25410 |
| 1022 | 16S rRNA (uracill498-N3)-methyltransferase | | CAETHG_2894 | CLJU_c08010 | CLRAG_08350 |
| 1023 | hypothetical protein | | CAETHG_2899 | CLJU_c08050 | CLRAG_08310 |
| 1024 | sporulation protein YqfC | | CAETHG_2900 | CLJU_c08060 | CLRAG_08300 |
| 1025 | probable rRNA maturation factor | | CAETHG_2903 | CLJU_c08080 | CLRAG_08270 |
| 1026 | protein of unknown function (DUF4342) | | CAETHG_2907 | CLJU_c08120 | CLRAG_08230 |
| 1027 | hypothetical protein | | CAETHG_2914 | CLJU_c08190 | CLRAG_18740 |
| 1028 | PH domain-containing protein | | CAETHG_2918 | CLJU_c08230 | CLRAG_08160 |
| 1029 | tRNA (adenine22-N1)-methyltransferase | | CAETHG_2919 | CLJU_c08240 | CLRAG_08150 |
| 1030 | hypothetical protein | | CAETHG_2921 | CLJU_c08260 | CLRAG_08130 |
| 1031 | hypothetical protein | | CAETHG_2923 | CLJU_c08280 | CLRAG_08100 |
| 1032 | hypothetical protein | | CAETHG_2940 | CLJU_c08470 | CLRAG_07990 |
| 1033 | hypothetical protein | | CAETHG_2956 | CLJU_c08620 | CLRAG_07870 |
| 1034 | hypothetical protein | | CAETHG_2958 | CLJU_c08640 | CLRAG_07850 |
| 1035 | NADH-quinone oxidoreductase subunit F | | CAETHG_2962 | CLJU_c08680 | CLRAG_07810 |
| 1036 | hypothetical protein | | CAETHG_2970 | CLJU_c08760 | CLRAG_07730 |
| 1037 | hypothetical protein | | CAETHG_2977 | CLJU_c08820 | CLRAG_07670 |
| 1038 | hypothetical protein | | CAETHG_2982 | CLJU_c08870 | CLRAG_07610 |
| 1039 | Ig-like domain-containing protein | | CAETHG_2993 | CLJU_c08990 | CLRAG_07520 |
| 1040 | Uncharacterized protein YpuA, DUF1002 family | | CAETHG_2995 | CLJU_c09010 | CLRAG_14010 |
| 1041 | hypothetical protein | | CAETHG_2996 | CLJU_c09020 | CLRAG_14000 |
| 1042 | Protein of unknown function (DUF1292) | | CAETHG_3030 | CLJU_c09350 | CLRAG_13750 |
| 1043 | hypothetical protein | | CAETHG_3033 | CLJU_c09380 | CLRAG_13720 |
| 1044 | FlgN protein | | CAETHG_3045 | CLJU_c09500 | CLRAG_13600 |
| 1045 | flagellar assembly factor FliW | | CAETHG_3048 | CLJU_c09530 | CLRAG_13570 |
| 1046 | hypothetical protein | | CAETHG_3051 | CLJU_c09560 | CLRAG_13550 |
| 1047 | hypothetical protein | | CAETHG_3057 | CLJU_c09620 | CLRAG_13520 |
| 1048 | IS66 C-terminal element | | CAETHG_3069 | CLJU_c09760, | CLRAG_16790 |
| | | | | CLJU_c05380 | |
| 1049 | hypothetical protein | | CAETHG_3077 | CLJU_c09840, | CLRAG_16740 |
| | | | | CLJU_c05400 | |
| 1050 | hypothetical protein | | CAETHG_3095 | CLJU_c10040 | CLRAG_13320 |
| 1051 | Bacterial Ig-like domain | | CAETHG_3098 | CLJU_c10080 | CLRAG_13290 |
| 1052 | Uncharacterized membrane protein, DUF441 | | CAETHG_3100 | CLJU_c10100 | CLRAG_13270 |
| | family | | | | |
| 1053 | Protein of unknown function (DUF3867) | | CAETHG_3101 | CLJU_c10110 | CLRAG_13260 |
| 1054 | phage uncharacterized protein TIGR01671 | | CAETHG_3102 | CLJU_c10120 | CLRAG_13250 |
| 1055 | hypothetical protein | | CAETHG_3103 | CLJU_c10130 | CLRAG_13240 |
| 1056 | hypothetical protein | | CAETHG_3107 | CLJU_c10170 | CLRAG_13200 |
| 1057 | hypothetical protein | | CAETHG_3132 | CLJU_c10420 | CLRAG_12950 |
| 1058 | hypothetical protein | | CAETHG_3133 | CLJU_c10430 | CLRAG_12940 |
| 1059 | hypothetical protein | | CAETHG_3136 | CLJU_c10460 | CLRAG_12910 |
| 1060 | hypothetical protein | | CAETHG_3139 | CLJU_c10490 | CLRAG_12880 |
| 1061 | MraZ protein | | CAETHG_3143 | CLJU_c10530 | CLRAG_12810 |
| 1062 | cell division protein FtsL | | CAETHG_3145 | CLJU_c10550 | CLRAG_12790 |
| 1063 | hypothetical protein | | CAETHG_3155 | CLJU_c10650 | CLRAG_12690 |
| 1064 | cell division inhibitor SepF | | CAETHG_3156 | CLJU_c10660 | CLRAG_12680 |
| 1065 | YggT family protein | | CAETHG_3157 | CLJU_c10670 | CLRAG_12670 |
| 1066 | hypothetical protein | | CAETHG_3167 | CLJU_c10770 | CLRAG_12570 |
| 1067 | Protein-S-isoprenylcysteine O-methyltransferase | | CAETHG_3171 | CLJU_c10820 | CLRAG_22440 |
| | Ste14 | | | | |
| 1068 | cyclic lactone autoinducer peptide | | CAETHG_3176 | CLJU_c28500, | CLRAG_03770 |
| | | | | CLJU_c25570 | |
| 1069 | hypothetical protein | | CAETHG_3178 | CLJU_c10880 | CLRAG_02280 |
| 1070 | type IV pilus assembly protein PilA | | CAETHG_3181 | CLJU_c10920 | CLRAG_12540 |
| 1071 | prepilin-type N-terminal cleavage/methylation | | CAETHG_3182 | CLJU_c10930 | CLRAG_12530 |
| | domain-containing protein | | | | |
| 1072 | prepilin-type N-terminal cleavage/methylation | | CAETHG_3184 | CLJU_c10950 | CLRAG_12510 |
| | domain-containing protein | | | | |
| 1073 | hypothetical protein | | CAETHG_3185 | CLJU_c10960 | CLRAG_12500 |
| 1074 | hypothetical protein | | CAETHG_3186 | CLJU_c10970 | CLRAG_12490 |
| 1075 | hypothetical protein | | CAETHG_3187 | CLJU_c10980 | CLRAG_12480 |
| 1076 | hypothetical protein | | CAETHG_3188 | CLJU_c10990 | CLRAG_12470 |
| 1077 | hypothetical protein | | CAETHG_3191 | CLJU_c11020 | CLRAG_12440 |
| 1078 | hypothetical protein | | CAETHG_3213 | CLJU_c11230 | CLRAG_12230 |
| 1079 | sporulation protein YtfJ | | CAETHG_3222 | CLJU_c11310 | CLRAG_12140 |
| 1080 | Protein of unknown function (DUF2953) | | CAETHG_3223 | CLJU_c11320 | CLRAG_12130 |
| 1081 | hypothetical protein | | CAETHG_3225 | CLJU_c11340 | CLRAG_12110 |
| 1082 | hypothetical protein | | CAETHG_3234 | CLJU_c11430 | CLRAG_12020 |
| 1083 | protein of unknown function (DUF4397) | | CAETHG_3235 | CLJU_c11440 | CLRAG_12010 |
| 1084 | DOmain of unknown function (DUF4883) | | CAETHG_3236 | CLJU_c11450 | CLRAG_12000 |
| 1085 | DNA-binding transcriptional regulator, FrmR family | | CAETHG_3238 | CLJU_c11470 | CLRAG_11980 |
| 1086 | Uncharacterized membrane protein YjjB, DUF3815 | | CAETHG_3239 | CLJU_c11480 | CLRAG_11970 |
| | family | | | | |
| 1087 | Uncharacterized membrane protein YjjP, DUF1212 | | CAETHG_3240 | CLJU_c11490 | CLRAG_11960 |
| | family | | | | |
| 1088 | hypothetical protein | | CAETHG_3244 | CLJU_c11530 | CLRAG_11920 |
| 1089 | PAP2 superfamily protein | | CAETHG_3267 | CLJU_c11760 | CLRAG_11780 |
| 1090 | hypothetical protein | | CAETHG_3270 | CLJU_c11790 | CLRAG_11750 |
| 1091 | Flavin reductase like domain-containing protein | | CAETHG_3273 | CLJU_c11820 | CLRAG_11720 |
| 1092 | Uncharacterized protein YrrD, contains PRC-barrel | | CAETHG_3295 | CLJU_c12130 | CLRAG_11670 |
| | domain | | | | |
| 1093 | Uncharacterized protein, UPF0297 family | | CAETHG_3298 | CLJU_c12160 | CLRAG_11640 |
| 1094 | Protein of unknown function (DUF1292) | | CAETHG_3300 | CLJU_c12180 | CLRAG_11620 |
| 1095 | Cell fate regulator YlbF, YheA/YmcA/DUF963 | | CAETHG_3303 | CLJU_c12210 | CLRAG_11590 |
| | family (controls sporulation, competence, biofilm | | | | |
| | development) | | | | |
| 1096 | conserved hypothetical protein | | CAETHG_3318 | CLJU_c12360 | CLRAG_11440 |
| 1097 | hypothetical protein | | CAETHG_3326 | CLJU_c12440 | CLRAG_11360 |
| 1098 | putative radical SAM enzyme, TIGR03279 family | | CAETHG_3328 | CLJU_c12460 | CLRAG_11340 |
| 1099 | TIGR00255 family protein | | CAETHG_3332 | CLJU_c12500 | CLRAG_11300 |
| 1100 | hypothetical protein | | CAETHG_3333 | CLJU_c12510 | CLRAG_11290 |
| 1101 | 16S rRNA (guanine966-N2)-methyltransferase | | CAETHG_3352 | CLJU_c12710 | CLRAG_11100 |
| 1102 | hypothetical protein | | CAETHG_3354 | CLJU_c12730 | CLRAG_11080 |
| 1103 | Predicted nucleotidyltransferase | | CAETHG_3357 | CLJU_c12750 | CLRAG_11050 |
| 1104 | uncharacterized protein | | CAETHG_3360 | CLJU_c12790 | CLRAG_11020 |
| 1105 | Histone acetyltransferase, component of the RNA | | CAETHG_3365 | CLJU_c12840 | CLRAG_10970 |
| | polymerase elongator complex | | | | |
| 1106 | hypothetical protein | | CAETHG_3369 | CLJU_c12870 | CLRAG_10930 |
| 1107 | hypothetical protein | | CAETHG_3372 | CLJU_c12900 | CLRAG_10900 |
| 1108 | putative endonuclease | | CAETHG_3379 | CLJU_c12970 | CLRAG_10830 |
| 1109 | ribosome maturation factor RimP | | CAETHG_3394 | CLJU_c13110 | CLRAG_10680 |
| 1110 | hypothetical protein | | CAETHG_3396 | CLJU_c13130 | CLRAG_10660 |
| 1111 | sporulation protein, YlmC/YmxH family | | CAETHG_3406 | CLJU_c13230 | CLRAG_10560 |
| 1112 | ribonucrease Y | | CAETHG_3412 | CLJU_c13290 | CLRAG_10500 |
| 1113 | EamA-like transporter family protein | | CAETHG_3416 | CLJU_c13330 | CLRAG_10460 |
| 1114 | hypothetical protein | | CAETHG_3419 | CLJU_c13360 | CLRAG_10430 |
| 1115 | hypothetical protein | | CAETHG_3422 | CLJU_c13380 | CLRAG_10410 |
| 1116 | BlaI family transcriptional regulator, penicillinase | | CAETHG_3431 | CLJU_c13550, | CLRAG_10320 |
| | repressor | | | CLJU_c13470 | |
| 1117 | protein of unknown function (DUF4179) | | CAETHG_3441 | CLJU_c13580 | CLRAG_10280 |
| 1118 | hypothetical protein | | CAETHG_3447 | CLJU_c13650 | CLRAG_10220 |
| 1119 | uncharacterized protein, PH0010 | | CAETHG_3448 | CLJU_c13660 | CLRAG_10210 |
| | family/AmmeMemoRadiSam system protein | | | | |
| | A/AmmeMemoRadiSam system protein B | | | | |
| 1120 | Branched-chain amino acid transport protein | | CAETHG_3452 | CLJU_c13700 | CLRAG_10170 |
| 1121 | Phenylpyruvate tautomerase PptA, 4- | | CAETHG_3455 | CLJU_c13730 | CLRAG_10120 |
| | oxalocrotonate tautomerase family | | | | |
| 1122 | Predicted PurR-regulated permease PerM | | CAETHG_3457 | CLJU_c13750 | CLRAG_10100 |
| 1123 | hypothetical protein | | CAETHG_3467 | CLJU_c13840 | CLRAG_10000 |
| 1124 | Uncharacterized protein, pyridoxamine 5′- | | CAETHG_3468 | CLJU_c13850 | CLRAG_09990 |
| | phosphate oxidase (PNPOx-like) family | | | | |
| 1125 | hypothetical protein | | CAETHG_3475, | CLJU_c13930, | CLRAG_09230 |
| | | | CAETHG_3476 | CLJU_c13920 | |
| 1126 | Coenzyme PQQ synthesis protein D (PqqD) | | CAETHG_3478 | CLJU_c13950 | CLRAG_09260 |
| 1127 | Winged helix-turn-helix DNA-binding | | CAETHG_3482 | CLJU_c13990 | CLRAG_09320 |
| 1128 | Protein of unknown function (DUF3793) | | CAETHG_3485 | CLJU_c14010 | CLRAG_09340 |
| 1129 | Cupin domain protein | | CAETHG_3502 | CLJU_c14200 | CLRAG_09530 |
| 1130 | hypothetical protein | | CAETHG_3548 | CLJU_c34970 | CLRAG_16880 |
| 1131 | L-cysteine desulfidase | | CAETHG_3563 | CLJU_c14640 | CLRAG_09920 |
| 1132 | diguanylate cyclase (GGDEF) domain-containing | | CAETHG_3564 | CLJU_c14650 | CLRAG_20550 |
| | protein | | | | |
| 1133 | diguanylate cyclase (GGDEF) domain-containing | | CAETHG_3580 | CLJU_c14800 | CLRAG_20310 |
| | protein | | | | |
| 1134 | conserved domain-containing protein | | CAETHG_3591 | CLJU_c14850 | CLRAG_20270 |
| 1135 | conserved domain-containing protein | | CAETHG_3592 | CLJU_c14860 | CLRAG_20260 |
| 1136 | Uncharacterized membrane protein YczE | | CAETHG_3593 | CLJU_c14870 | CLRAG_20250 |
| 1137 | hypothetical protein | | CAETHG_3612 | CLJU_c15100 | CLRAG_24270 |
| 1138 | hypothetical protein | | CAETHG_3613 | CLJU_c15110 | CLRAG_24230 |
| 1139 | 5-methylcytosine-specific restriction enzyme B | | CAETHG_3617 | CLJU_c15150 | CLRAG_24200 |
| 1140 | hypothetical protein | | CAETHG_3621 | CLJU_c15190 | CLRAG_24160 |
| 1141 | hypothetical protein | | CAETHG_3622 | CLJU_c15200 | CLRAG_24150 |
| 1142 | ABC-2 type transport system permease protein | | CAETHG_3640 | CLJU_c15380 | CLRAG_24080 |
| 1143 | ABC-2 type transport system permease protein | | CAETHG_3641 | CLJU_c15400 | CLRAG_24070 |
| 1144 | HlyD family secretion protein | | CAETHG_3642 | CLJU_c15410 | CLRAG_24060 |
| 1145 | hypothetical protein | | CAETHG_3644 | CLJU_c15430 | CLRAG_24040 |
| 1146 | Pentapeptide repeat-containing protein | | CAETHG_3657 | CLJU_c15620 | CLRAG_32630 |
| 1147 | C_GCAxxG_C_C family probable redox protein | | CAETHG_3662 | CLJU_c15630 | CLRAG_32680 |
| 1148 | Glycopeptide antibiotics resistance protein | | CAETHG_3675 | CLJU_c15680 | CLRAG_32830 |
| 1149 | protein of unknown function (DUF4367) | | CAETHG_3679 | CLJU_c15710 | CLRAG_32870 |
| 1150 | O-methyltransferase | | CAETHG_3683 | CLJU_c15750 | CLRAG_32910 |
| 1151 | putative sporulation protein YtaF | | CAETHG_3694 | CLJU_c15940 | CLRAG_32970 |
| 1152 | putative conserved protein UCP010219 | | CAETHG_3700 | CLJU_c16060 | CLRAG_33030 |
| 1153 | protein of unknown function (DUF4363) | | CAETHG_3732 | CLJU_c16380 | CLRAG_33180 |
| 1154 | Uncharacterized membrane protein YcaP, DUF421 | | CAETHG_3733 | CLJU_c16390 | CLRAG_33190 |
| | family | | | | |
| 1155 | protein of unknown function (DUF3870) | | CAETHG_3734 | CLJU_c16400 | CLRAG_33200 |
| 1156 | hypothetical protein | | CAETHG_3818 | CLJU_c17060 | CLRAG_33930 |
| 1157 | Peptidase A4 family protein | | CAETHG_3820 | CLJU_c17080 | CLRAG_33950 |
| 1158 | hypothetical protein | | CAETHG_3821 | CLJU_c17090 | CLRAG_33960 |
| 1159 | hypothetical protein | | CAETHG_3836, | CLJU_c17240, | CLRAG_34210 |
| | | | CAETHG_3837 | CLJU_c17230 | |
| 1160 | DNA-binding transcriptional regulator, MerR family | | CAETHG_3839 | CLJU_c17260 | CLRAG_34230 |
| 1161 | protein of unknown function (DUF4342) | | CAETHG_3843 | CLJU_c17300 | CLRAG_29280 |
| 1162 | 23S rRNA (pseudouridine1915-N3)- | | CAETHG_3853 | CLJU_c17400 | CLRAG_01290 |
| | methyltransferase | | | | |
| 1163 | hypothetical protein | | CAETHG_3858 | CLJU_c17450 | CLRAG_01260 |
| 1164 | hypothetical protein | | CAETHG_3869 | CLJU_c17610 | CLRAG_01130 |
| 1165 | Protein of unknown function (DUF3795) | | CAETHG_3873 | CLJU_c17650 | CLRAG_01090 |
| 1166 | two-component system, response regulator YcbB | | CAETHG_3881 | CLJU_c17730 | CLRAG_00990 |
| 1167 | hypothetical protein | | CAETHG_3883 | CLJU_c17750 | CLRAG_00970 |
| 1168 | hypothetical protein | | CAETHG_3887 | CLJU_c17790 | CLRAG_00920 |
| 1169 | hypothetical protein | | CAETHG_3888 | CLJU_c17800 | CLRAG_00910 |
| 1170 | Peptidase family M28 | | CAETHG_3891 | CLJU_c17830 | CLRAG_00880 |
| 1171 | CheY chemotaxis protein or a CheY-like REC | | CAETHG_3894 | CLJU_c17860 | CLRAG_00800 |
| | (receiver) domain | | | | |
| 1172 | 3-methyladenine DNA glycosylase AlkD | | CAETHG_3896 | CLJU_c17880 | CLRAG_00780 |
| 1173 | EamA domain-containing membrane protein RarD | | CAETHG_3897 | CLJU_c17890 | CLRAG_00760 |
| 1174 | xanthine dehydrogenase accessory factor | | CAETHG_3903 | CLJU_c17940 | CLRAG_00690 |
| 1175 | diguanylate cyclase (GGDEF) domain-containing | | CAETHG_3905 | CLJU_c17960 | CLRAG_00670 |
| | protein | | | | |
| 1176 | Uncharacterized conserved protein YraI | | CAETHG_3911 | CLJU_c18020 | CLRAG_00620 |
| 1177 | Yip1 domain-containing protein | | CAETHG_3916 | CLJU_c18070 | CLRAG_00580 |
| 1178 | SatD family (SatD) | | CAETHG_3926 | CLJU_c18180 | CLRAG_00500 |
| 1179 | SatD family (SatD) | | CAETHG_3927 | CLJU_c18190 | CLRAG_00490 |
| 1180 | Protein of unknown function (DUF3307) | | CAETHG_3928 | CLJU_c18200 | CLRAG_00480 |
| 1181 | EamA-like transporter family protein | | CAETHG_3929 | CLJU_c18210 | CLRAG_00470 |
| 1182 | hypothetical protein | | CAETHG_3930 | CLJU_c18220 | CLRAG_00390 |
| 1183 | Uncharacterized membrane protein | | CAETHG_3931 | CLJU_c18230 | CLRAG_00380 |
| 1184 | hypothetical protein | | CAETHG_3946 | CLJU_c18390 | CLRAG_00290 |
| 1185 | hypothetical protein | | CAETHG_3947 | CLJU_c18400 | CLRAG_00280 |
| 1186 | hypothetical protein | | CAETHG_3948 | CLJU_c18410 | CLRAG_00270 |
| 1187 | hypothetical protein | | CAETHG_3950 | CLJU_c18430 | CLRAG_00250 |
| 1188 | hypothetical protein | | CAETHG_3955 | CLJU_c18480 | CLRAG_00200 |
| 1189 | Glucose inhibited division protein A | | CAETHG_3958 | CLJU_c18510 | CLRAG_00130 |
| 1190 | protein of unknown function (DUF2935) | | CAETHG_3971 | CLJU_c18630 | CLRAG_00020 |
| 1191 | hypothetical protein | 2.7.4.12, | CAETHG_3972 | CLJU_c18640 | CLRAG_00010 |
| | | 2.7.4.9 | | | |
| 1192 | hypothetical protein | | CAETHG_3975 | CLJU_c34960 | CLRAG_26220 |
| 1193 | hypothetical protein | | CAETHG_3982 | CLJU_c18680 | CLRAG_16680 |
| 1194 | hypothetical protein | | CAETHG_3983 | CLJU_c18690 | CLRAG_16670 |
| 1195 | hypothetical protein | | CAETHG_3984 | CLJU_c18700 | CLRAG_16650 |
| 1196 | protein of unknown function (DUF4397) | | CAETHG_3992 | CLJU_c18730 | CLRAG_16560 |
| 1197 | hypothetical protein | | CAETHG_4006 | CLJU_c18830 | CLRAG_16420 |
| 1198 | Uncharacterized membrane-anchored protein YitT, | | CAETHG_4049 | CLJU_c19150 | CLRAG_39900 |
| | contains DUF161 and DUF2179 domains | | | | |
| 1199 | hypothetical protein | | CAETHG_4051 | CLJU_c19170 | CLRAG_39880 |
| 1200 | hypothetical protein | | CAETHG_4053 | CLJU_c19180 | CLRAG_39870 |
| 1201 | Immunity protein 22 | | CAETHG_4059 | CLJU_c19240 | CLRAG_39830 |
| 1202 | hypoxanthine phosphoribosyltransferase | 2.4.2.22, | CAETHG_1290 | CLJU_c33920 | CLRAG_14070 |
| | | 2.4.2.7, | | | |
| | | 2.4.2.8 | | | |
| 1203 | hypoxanthine phosphoribosyltransferase | 2.4.2.22, | CAETHG_1988 | CLJU_c41540 | CLRAG_04670 |
| | | 2.4.2.7, | | | |
| | | 2.4.2.8 | | | |
| 1204 | cyclase | | CAETHG_3264 | CLJU_c11730 | CLRAG_11810 |
| 1205 | glutamine amidotransferase | | CAETHG_3261 | CLJU_c11700 | CLRAG_11840 |
| 1206 | imidazoleglycerol-phosphate dehydratase | 4.2.1.19 | CAETHG_3260 | CLJU_c11690 | CLRAG_11850 |
| 1207 | imidazolonepropionase | 3.5.2.7 | CAETHG_0233 | CLJU_c21470 | CLRAG_31030 |
| 1208 | indole-3-glycerol phosphate synthase | 4.1.1.48 | CAETHG_3704 | CLJU_c16100 | CLRAG_33070 |
| 1209 | IMP dehydrogenase | 1.1.1.205 | CAETHG_1571 | CLJU_c37180 | CLRAG_36620 |
| 1210 | iron (metal) dependent repressor, DtxR family | | CAETHG_1335 | CLJU_c34350 | CLRAG_17700 |
| 1211 | [FeFe] hydrogenase H-cluster maturation GTPase | | CAETHG_2063 | CLJU_c42380 | CLRAG_05500 |
| | HydF | | | | |
| 1212 | epoxyqueuosine reductase | | CAETHG_1774 | CLJU_c39290 | CLRAG_21500 |
| 1213 | beta-aspartyl-dipeptidase (metallo-type) | 3.4.11.1, | CAETHG_0748 | CLJU_c26670 | CLRAG_08540 |
| | | 3.4.11.2, | | | |
| | | 3.4.13.3, | | | |
| | | 3.4.11.23 | | | |
| 1214 | Isoleucyl-tRNA synthetase | | CAETHG_2292 | CLJU_c01890 | CLRAG_27490 |
| 1215 | ketopantoate reductase | 1.1.1.169 | CAETHG_0118, | CLJU_c08720, | CLRAG_25930 |
| | | | CAETHG_2966 | CLJU_c20360 | |
| 1216 | 2-dehydropantoate 2-reductase | | CAETHG_3877 | CLJU_c17690 | CLRAG_01040 |
| 1217 | L-arabinose isomerase | 5.3.1.4 | CAETHG_2228 | CLJU_c01200 | CLRAG_30220 |
| 1218 | L-aspartate oxidase | | CAETHG_0502 | CLJU_c24420 | CLRAG_25150 |
| 1219 | L-ribulose 5-phosphate 4-epimerase | | CAETHG_2229 | CLJU_c01210 | CLRAG_30210 |
| 1220 | L-serine dehydratase | 4.3.1.17, | CAETHG_1224 | CLJU_c33250 | CLRAG_15010 |
| | | 4.3.1.15, | | | |
| | | 4.3.1.19, | | | |
| | | 4.2.1.13 | | | |
| 1221 | L-serine dehydratase | 4.3.1.17, | CAETHG_1225 | CLJU_c33260 | CLRAG_15000 |
| | | 4.3.1.15, | | | |
| | | 4.3.1.19, | | | |
| | | 4.2.1.13 | | | |
| 1222 | L-seryl-tRNA(Sec) selenium transferase | | CAETHG_2839 | CLJU_c07470, | CLRAG_32220 |
| | | | | CLJU_c27710 | |
| 1223 | L-threonine aldolase | 4.1.2.5 | CAETHG_0686 | CLJU_c26170 | CLRAG_04250 |
| 1224 | threonine-phosphate decarboxylase | 4.1.1.81 | CAETHG_1128 | CLJU_c32000 | CLRAG_02630 |
| 1225 | LemA protein | | CAETHG_0069 | CLJU_c19890 | CLRAG_39190 |
| 1226 | leucyl-tRNA synthetase | | CAETHG_2377 | CLJU_c02750 | CLRAG_28350 |
| 1227 | repressor LexA | | CAETHG_0188 | CLJU_c21030 | CLRAG_18990 |
| 1228 | Predicted dehydrogenase | 1.1.1.18 | CAETHG_1307 | CLJU_c34090 | CLRAG_14240 |
| 1229 | lipoic acid synthetase | | CAETHG_1220 | CLJU_c33210 | CLRAG_15050 |
| 1230 | LL-diaminopimelate aminotransferase apoenzyme | 2.6.1.83 | CAETHG_3510 | CLJU_c14280 | CLRAG_09600 |
| 1231 | large subunit ribosomal protein L10 | | CAETHG_1957 | CLJU_c41140 | CLRAG_23330 |
| 1232 | LSU ribosomal protein L11P | | CAETHG_1959 | CLJU_c41160 | CLRAG_23350 |
| 1233 | LSU ribosomal protein L13P | | CAETHG_1914 | CLJU_c40710 | CLRAG_22900 |
| 1234 | LSU ribosomal protein L14P | | CAETHG_1937 | CLJU_c40940 | CLRAG_23130 |
| 1235 | LSU ribosomal protein L15P | | CAETHG_1928 | CLJU_c40850 | CLRAG_23040 |
| 1236 | LSU ribosomal protein L16P | | CAETHG_1940 | CLJU_c40970 | CLRAG_23160 |
| 1237 | LSU ribosomal protein L17P | | CAETHG_1919 | CLJU_c40760 | CLRAG_22950 |
| 1238 | LSU ribosomal protein L20P | | CAETHG_1344 | CLJU_c34440 | CLRAG_14520 |
| 1239 | LSU ribosomal protein L22P | | CAETHG_1942 | CLJU_c40990 | CLRAG_23180 |
| 1240 | LSU ribosomal protein L24P | | CAETHG_1936 | CLJU_c40930 | CLRAG_23120 |
| 1241 | LSU ribosomal protein L27P | | CAETHG_2830 | CLJU_c07380 | CLRAG_26460 |
| 1242 | large subunit ribosomal protein L28 | | CAETHG_3348 | CLJU_c12670 | CLRAG_11140 |
| 1243 | LSU ribosomal protein L29P | | CAETHG_1939 | CLJU_c40960 | CLRAG_23150 |
| 1244 | large subunit ribosomal protein L30 | | CAETHG_1929 | CLJU_c40860 | CLRAG_23050 |
| 1245 | LSU ribosomal protein L32P | | CAETHG_3361 | CLJU_c12800 | CLRAG_11010 |
| 1246 | large subunit ribosomal protein L33 | | CAETHG_1962 | CLJU_c41190 | CLRAG_23380 |
| 1247 | large subunit ribosomal protein L34 | | CAETHG_2123 | CLJU_c42970 | CLRAG_25780 |
| 1248 | LSU ribosomal protein L36P | | CAETHG_1924 | CLJU_c40810 | CLRAG_23000 |
| 1249 | large subunit ribosomal protein L4 | | CAETHG_1946 | CLJU_c41030 | CLRAG_23220 |
| 1250 | large subunit ribosomal protein L9 | | CAETHG_2098 | CLJU_c42730 | CLRAG_25540 |
| 1251 | lysine:proton symporter, AAT family | | CAETHG_0271, | CLJU_c24360, | CLRAG_31350 |
| | | | CAETHG_0496 | CLJU_c21810 | |
| 1252 | DNA-binding transcriptional regulator, LysR family | | CAETHG_3878 | CLJU_c17700 | CLRAG_01030 |
| 1253 | lysyl-tRNA synthetase, class II | | CAETHG_1982 | CLJU_c41480 | CLRAG_04610 |
| 1254 | Sugar phosphate permease | | CAETHG_3582 | CLJU_c14820 | CLRAG_20290 |
| 1255 | Malate/lactate/ureidoglycolate dehydrogenase, | 1.1.1.37 | CAETHG_2689 | CLJU_c05920 | CLRAG_07150 |
| | LDH2 family | | | | |
| 1256 | [acyl-carrier-protein] S-malonyltransferase | | CAETHG_2048 | CLJU_c42170 | CLRAG_05280 |
| 1257 | Mn-containing catalase | | CAETHG_3970 | CLJU_c18620 | CLRAG_00030 |
| 1258 | manganese-dependent inorganic pyrophosphatase | 3.6.1.1 | CAETHG_3137 | CLJU_c10470 | CLRAG_12900 |
| 1259 | mannose-1-phosphate guanylyltransferase | 2.7.7.22 | CAETHG_2296 | CLJU_c01930 | CLRAG_27530 |
| 1260 | mannose-6-phosphate isomerase, type 1 | 5.3.1.8 | CAETHG_1790 | CLJU_c39450 | CLRAG_21660 |
| 1261 | DNA-binding transcriptional regulator, MerR family | | CAETHG_0698 | CLJU_c26220 | CLRAG_04300 |
| 1262 | HDIG domain-containing protein | | CAETHG_1005 | CLJU_c30060 | CLRAG_15690 |
| 1263 | methylenetetra hydrofolate dehydrogenase | 1.5.1.5 | CAETHG_1616 | CLJU_c37630 | CLRAG_37040 |
| | (NADP+)/methenyltetrahydrofolate | | | | |
| | cyclohydrolase | | | | |
| 1264 | methionine adenosyltransferase | 2.5.1.6 | CAETHG_0419, | CLJU_c23550, | CLRAG_28180 |
| | | | CAETHG_2358 | CLJU_c02580 | |
| 1265 | methionyl aminopeptidase | 3.4.11.1, | CAETHG_1486 | CLJU_c35780 | CLRAG_06310 |
| | | 3.4.11.2, | | | |
| | | 3.4.13.3, | | | |
| | | 3.4.11.23 | | | |
| 1266 | 5-methyltetrahydrofolate--homocysteine | 2.1.1.13, | CAETHG_2755 | CLJU_c06650 | CLRAG_18450 |
| | methyltransferase | 2.1.1.14 | | | |
| 1267 | 5-methyltetrahydrofolate--homocysteine | | CAETHG_2843, | CLJU_c07550, | CLRAG_34280 |
| | methyltransferase | | CAETHG_2848 | CLJU_c07500 | |
| 1268 | methionyl-tRNA formyltransferase | | CAETHG_3339 | CLJU_c12570 | CLRAG_11230 |
| 1269 | methionyl-tRNA synthetase | | CAETHG_2275 | CLJU_c01730 | CLRAG_27330 |
| 1270 | methyl-accepting chemotaxis sensory transducer | | CAETHG_0308 | CLJU_c22100 | CLRAG_31620 |
| 1271 | methyl-accepting chemotaxis sensory transducer | | CAETHG_0077 | CLJU_c19970 | CLRAG_39070 |
| | with Cache sensor | | | | |
| 1272 | methyl-accepting chemotaxis sensory transducer | | CAETHG_0229 | CLJU_c21430 | CLRAG_30990 |
| | with Cache sensor | | | | |
| 1273 | methyl-accepting chemotaxis sensory transducer | | CAETHG_0350 | CLJU_c22880 | CLRAG_01910 |
| | with Cache sensor | | | | |
| 1274 | methyl-accepting chemotaxis sensory transducer | | CAETHG_0542, | CLJU_c24770, | CLRAG_18050 |
| | with Cache sensor | | CAETHG_1048 | CLJU_c30430 | |
| 1275 | methyl-accepting chemotaxis sensory transducer | | CAETHG_2402 | CLJU_c02870 | CLRAG_28470 |
| | with Cache sensor | | | | |
| 1276 | methyl-accepting chemotaxis sensory transducer | | CAETHG_2997 | CLJU_c09030 | CLRAG_13990 |
| | with Cache sensor | | | | |
| 1277 | methyl-accepting chemotaxis sensory transducer | | CAETHG_3106 | CLJU_c10160 | CLRAG_13210 |
| | with Cache sensor | | | | |
| 1278 | methyl-accepting chemotaxis sensory transducer | | CAETHG_3430 | CLJU_c13460 | CLRAG_10330 |
| | with Cache sensor | | | | |
| 1279 | methyl-accepting chemotaxis sensory transducer | | CAETHG_3459 | CLJU_c13760 | CLRAG_10080 |
| | with Cache sensor | | | | |
| 1280 | methyl-accepting chemotaxis sensory transducer | | CAETHG_3466 | CLJU_c13830 | CLRAG_10010 |
| | with Cache sensor | | | | |
| 1281 | methyl-accepting chemotaxis sensory transducer | | CAETHG_4020 | CLJU_c18880 | CLRAG_40120 |
| | with Cache sensor | | | | |
| 1282 | methylaspartate ammonia-lyase | | CAETHG_1904 | CLJU_c40610 | CLRAG_22800 |
| 1283 | methylated-DNA-[protein]-cysteine S- | | CAETHG_3895 | CLJU_c17870 | CLRAG_00790 |
| | methyltransferase | | | | |
| 1284 | 5,10-methylenetetrahydrofolate reductase | 1.5.1.20 | CAETHG_1614 | CLJU_c37610 | CLRAG_37020 |
| 1285 | methylglyoxal synthase | | CAETHG_2822 | CLJU_c07300 | CLRAG_26540 |
| 1286 | methyltetrahydrofolate--corrinoid iron-sulfur | | CAETHG_1609 | CLJU_c37560 | CLRAG_36970 |
| | protein Co-methyltransferase | | | | |
| 1287 | adenosylhomocysteine nucleosidase | 3.2.2.16, | CAETHG_3160 | CLJU_c10700 | CLRAG_12640 |
| | | 3.2.2.9 | | | |
| 1288 | Methyltransferase domain-containing protein | | CAETHG_1633 | CLJU_c38050 | CLRAG_37420 |
| 1289 | Mo-nitrogenase MoFe protein subunit NifD | | CAETHG_2570 | CLJU_c04930 | CLRAG_38320 |
| | precursor | | | | |
| 1290 | nitrogenase molybdenum-iron protein beta chain | | CAETHG_2571 | CLJU_c04940 | CLRAG_38330 |
| 1291 | Modular FeS cluster scaffolding protein NifU | | CAETHG_3294 | CLJU_c12120 | CLRAG_11680 |
| 1292 | molybdopterin-guanine dinucleotide biosynthesis | | CAETHG_0227 | CLJU_c21410 | CLRAG_30970 |
| | protein A | | | | |
| 1293 | molybdopterin adenylyltransferase | | CAETHG_0574 | CLJU_c25060 | CLRAG_17720 |
| 1294 | molybdopterin-guanine dinucleotide biosynthesis | 1.1.99.33 | CAETHG_2792 | CLJU_c07010 | CLRAG_18860 |
| | protein B | | | | |
| 1295 | molybdopterin molybdotransferase | 1.1.99.33 | CAETHG_2791 | CLJU_c07000 | CLRAG_18850 |
| 1296 | monosaccharide ABC transporter ATP-binding | | CAETHG_1384 | CLJU_c34860 | CLRAG_26100 |
| | protein, CUT2 family | | | | |
| 1297 | ribose transport system ATP-binding protein | | CAETHG_2236 | CLJU_c01270 | CLRAG_30150 |
| 1298 | monosaccharide ABC transporter membrane | | CAETHG_1382 | CLJU_c34840 | CLRAG_26080 |
| | protein, CUT2 family | | | | |
| 1299 | simple sugar transport system permease protein | | CAETHG_1383 | CLJU_c34850 | CLRAG_26090 |
| 1300 | monosaccharide ABC transporter substrate- | | CAETHG_1385 | CLJU_c34870 | CLRAG_26110 |
| | binding protein, CUT2 family | | | | |
| 1301 | dsDNA-specific endonuclease/ATPase MutS2 | | CAETHG_3607 | CLJU_c15050 | CLRAG_24320 |
| 1302 | N-acetyl-gamma-glutamyl-phosphate reductase | 1.2.1.38 | CAETHG_0241 | CLJU_c21540 | CLRAG_31100 |
| 1303 | N-acetylglutamate kinase | 2.7.2.8 | CAETHG_0239 | CLJU_c21520 | CLRAG_31080 |
| 1304 | N-acetyl-anhydromuramyl-L-alanine amidase | 3.5.1.28 | CAETHG_1654 | CLJU_c32580 | CLRAG_29450 |
| | AmpD | | | | |
| 1305 | N-acetylmuramoyl-L-alanine amidase | 3.5.1.28 | CAETHG_1765 | CLJU_c39200 | CLRAG_21350 |
| 1306 | N-acetylmuramoyl-L-alanine amidase | | CAETHG_1912 | CLJU_c40690 | CLRAG_22880 |
| 1307 | N-acyl-D-amino-acid deacylase | | CAETHG_0452 | CLJU_c23870 | CLRAG_17300 |
| 1308 | amidohydrolase | | CAETHG_2511 | CLJU_c04420 | CLRAG_37750 |
| 1309 | N-carbamoyl-L-amino-acid hydrolase | 3.5.1.6 | CAETHG_1498 | CLJU_c35910 | CLRAG_06410 |
| 1310 | N-formylglutamate amidohydrolase | | CAETHG_0505 | CLJU_c24450 | CLRAG_30120 |
| 1311 | 4-hydroxybutyrate dehydrogenase | 1.1.1.1 | CAETHG_1741 | CLJU_c38930 | CLRAG_21110 |
| 1312 | NAD-dependent deacetylase | | CAETHG_2239 | CLJU_c01320 | CLRAG_26980 |
| 1313 | isocitrate dehydrogenase (NAD+) | 1.1.1.286, | CAETHG_2753 | CLJU_c06630 | CLRAG_18430 |
| | | 1.1.1.41 | | | |
| 1314 | malate dehydrogenase (oxaloacetate- | 1.1.1.37, | CAETHG_1702, | CLJU_c38460, | CLRAG_26900 |
| | decarboxylating) | 1.1.1.40, | CAETHG_2478 | CLJU_c04160 | |
| | | 1.1.1.38, | | | |
| | | 4.1.1.3, | | | |
| | | 1.1.1.39 | | | |
| 1315 | NAD(P)-dependent iron-only hydrogenase catalytic | | CAETHG_3569 | CLJU_c14700 | CLRAG_20490 |
| | subunit | | | | |
| 1316 | NADH-quinone oxidoreductase subunit E | | CAETHG_3571 | CLJU_c14720 | CLRAG_20470 |
| 1317 | NAD(P)H-dependent nitrate reductase catalytic | 1.7.7.2 | CAETHG_0437 | CLJU_c23730 | CLRAG_17440 |
| | subunit | | | | |
| 1318 | NAD(P)H-dependent nitrate reductase diaphorase | | CAETHG_0435 | CLJU_c23710 | CLRAG_17460 |
| | subunit | | | | |
| 1319 | NAD(P)H-dependent nitrate reductase iron-sulfur | | CAETHG_0436 | CLJU_c23720 | CLRAG_17450 |
| | subunit | | | | |
| 1320 | NADH-quinone oxidoreductase subunit E | 1.12.1.4, | CAETHG_2794 | CLJU_c07030 | CLRAG_18880 |
| | | 1.1.99.33 | | | |
| 1321 | NADH-quinone oxidoreductase subunit F | 1.12.1.4, | CAETHG_1577, | CLJU_c07040, | CLRAG_20480 |
| | | 1.1.99.33 | CAETHG_3570, | CLJU_c14710 | |
| | | | CAETHG_2795 | | |
| 1322 | 4Fe—4S dicluster domain-containing protein | 1.12.1.4, | CAETHG_2796 | CLJU_c07050 | CLRAG_18900 |
| | | 1.1.99.33 | | | |
| 1323 | NAD+ diphosphatase | | CAETHG_2205 | CLJU_c00910 | CLRAG_19670 |
| 1324 | glutamate dehydrogenase (NADP+) | | CAETHG_2367 | CLJU_c02660 | CLRAG_28260 |
| 1325 | NADPH-dependent FMN reductase | | CAETHG_0974 | CLJU_c29750 | CLRAG_35750 |
| 1326 | glutamate synthase (NADPH/NADH) small chain | | CAETHG_0477 | CLJU_c24190 | CLRAG_24880 |
| 1327 | carbon-monoxide dehydrogenase catalytic subunit | | CAETHG_1621 | CLJU_c37670 | CLRAG_37080 |
| 1328 | nicotinamidase/pyrazinamidase | 3.5.1.19 | CAETHG_0378 | CLJU_c23150 | CLRAG_01660 |
| 1329 | NAD+ kinase | 2.7.1.23 | CAETHG_3207 | CLJU_c11180 | CLRAG_12280 |
| 1330 | nicotinate phosphoribosyltransferase | 2.4.2.11 | CAETHG_3427 | CLJU_c13430 | CLRAG_10360 |
| 1331 | nicotinate-nucleotide adenylyltransferase | 2.7.7.1, | CAETHG_2832 | CLJU_c07400 | CLRAG_26440 |
| | | 2.7.7.18 | | | |
| 1332 | molybdenum transport protein | | CAETHG_1634 | CLJU_c38070 | CLRAG_37440 |
| 1333 | nicotinate-nucleotide pyrophosphorylase | | CAETHG_0501 | CLJU_c24410 | CLRAG_25140 |
| | [carboxylating] | | | | |
| 1334 | nicotinate-nucleotide-dimethylbenzimidazole | 2.4.2.21 | CAETHG_1122 | CLJU_c31940 | CLRAG_02570 |
| | phosphoribosyltransferase | | | | |
| 1335 | nitrogen regulatory protein P-II family | | CAETHG_2091, | CLJU_c04050, | CLRAG_05670 |
| | | | CAETHG_2468 | CLJU_c42650 | |
| 1336 | nitrogen regulatory protein P-II family | | CAETHG_2568 | CLJU_c04910 | CLRAG_38300 |
| 1337 | nitrogen regulatory protein P-II family | | CAETHG_2569 | CLJU_c04920 | CLRAG_38310 |
| 1338 | nitrogenase molybdenum-iron protein NifN | | CAETHG_2573 | CLJU_c04960 | CLRAG_38350 |
| 1339 | nitrogenase iron protein NifH | 1.18.6.1 | CAETHG_0368, | CLJU_c23050, | CLRAG_01760 |
| | | | CAETHG_0375 | CLJU_c23120 | |
| 1340 | nitrogenase iron protein NifH | 1.18.6.1 | CAETHG_0417, | CLJU_c23530, | CLRAG_38290 |
| | | | CAETHG_2567 | CLJU_c04900 | |
| 1341 | nitrogenase molybdenum-cofactor synthesis | | CAETHG_0374 | CLJU_c23110 | CLRAG_01700 |
| | protein NifE | | | | |
| 1342 | nitrogenase molybdenum-cofactor synthesis | | CAETHG_2572 | CLJU_c04950 | CLRAG_38340 |
| | protein NifE | | | | |
| 1343 | Nitroreductase | | CAETHG_0934 | CLJU_c29400 | CLRAG_35400 |
| 1344 | simple sugar transport system ATP-binding protein | | CAETHG_0998 | CLJU_c29990 | CLRAG_35990 |
| 1345 | nucleoside ABC transporter ATP-binding protein | | CAETHG_1808 | CLJU_c39620 | CLRAG_21810 |
| 1346 | simple sugar transport system permease protein | | CAETHG_0996, | CLJU_c29970, | CLRAG_35970 |
| | | | CAETHG_1806 | CLJU_c39600 | |
| 1347 | simple sugar transport system permease protein | | CAETHG_0997 | CLJU_c29980 | CLRAG_35980 |
| 1348 | nucleoside ABC transporter membrane protein | | CAETHG_1807 | CLJU_c39610 | CLRAG_21800 |
| 1349 | basic membrane protein A | | CAETHG_0999 | CLJU_c30000 | CLRAG_36000 |
| 1350 | nucleoside-binding protein | | CAETHG_1809 | CLJU_c39630 | CLRAG_21820 |
| 1351 | ATP-dependent Lon protease | | CAETHG_1470 | CLJU_c35620 | CLRAG_06150 |
| 1352 | nucleoside-triphosphatase | | CAETHG_3826 | CLJU_c17140 | CLRAG_34000 |
| 1353 | NusA antitermination factor | | CAETHG_3395 | CLJU_c13120 | CLRAG_10670 |
| 1354 | NusB antitermination factor | | CAETHG_3201 | CLJU_c11120 | CLRAG_12340 |
| 1355 | O-acetylhomoserine sulfhydrylase | 2.5.1.48, | CAETHG_2754 | CLJU_c06640 | CLRAG_18440 |
| | | 2.5.1.—, | | | |
| | | 4.2.99.8, | | | |
| | | 4.2.99.10, | | | |
| | | 2.5.1.49, | | | |
| | | 4.2.99.9 | | | |
| 1356 | N6-L-threonylcarbamoyladenine synthase | | CAETHG_1595 | CLJU_c37400 | CLRAG_36840 |
| 1357 | oligopeptidase F. Metallo peptidase. MEROPS | | CAETHG_4039 | CLJU_c19050 | CLRAG_39980 |
| | family M03B | | | | |
| 1358 | putative oligopeptide transporter, OPT family | | CAETHG_3477 | CLJU_c13940 | CLRAG_09250 |
| 1359 | ornithine carbamoyltransferase | 2.1.3.3 | CAETHG_0591 | CLJU_c25230 | CLRAG_03580 |
| 1360 | orotate phosphoribosyltransferase | 2.4.2.10 | CAETHG_1476 | CLJU_c35680 | CLRAG_06210 |
| 1361 | orotidine-5′-phosphate decarboxylase | 4.1.1.23 | CAETHG_1479 | CLJU_c35710 | CLRAG_06240 |
| 1362 | oxygen-independent coproporphyrinogen-3 | | CAETHG_2888 | CLJU_c07950 | CLRAG_25430 |
| | oxidase | | | | |
| 1363 | tripeptide aminopeptidase | | CAETHG_0005, | CLJU_c19290, | CLRAG_39770 |
| | | | CAETHG_0008 | CLJU_c19310 | |
| 1364 | peptide chain release factor 1 | | CAETHG_2331 | CLJU_c02260 | CLRAG_27860 |
| 1365 | peptide chain release factor 3 | | CAETHG_1685 | CLJU_c38280 | CLRAG_20750 |
| 1366 | peptide deformylase | | CAETHG_0293 | CLJU_c21960 | CLRAG_31510 |
| 1367 | peptide deformylase | | CAETHG_3338, | CLJU_c13640, | CLRAG_11240 |
| | | | CAETHG_3446 | CLJU_c12560 | |
| 1368 | peptide deformylase | | CAETHG_3892 | CLJU_c17840 | CLRAG_00870 |
| 1369 | peptidoglycan transpeptidase precursor, ErfK-YbiS- | | CAETHG_3681 | CLJU_c15730 | CLRAG_32890 |
| | YhnG family | | | | |
| 1370 | peptidyl-prolyl cis-trans isomerase B (cyclophilin B) | | CAETHG_0351 | CLJU_c22890 | CLRAG_01900 |
| 1371 | peptidyl-tRNA hydrolase, PTH1 family | | CAETHG_2002 | CLJU_c41730 | CLRAG_04860 |
| 1372 | phage shock protein A (PspA) family protein | | CAETHG_2260 | CLJU_c01540 | CLRAG_27200 |
| 1373 | phenylalanyl-tRNA synthetase beta subunit | | CAETHG_1341 | CLJU_c34410 | CLRAG_14490 |
| 1374 | phenylalanyl-tRNA synthetase, alpha subunit | | CAETHG_1342 | CLJU_c34420 | CLRAG_14500 |
| 1375 | phosphate ABC transporter ATP-binding protein, PhoT family | | CAETHG_3324 | CLJU_c12420 | CLRAG_11380 |
| 1376 | phosphate ABC transporter membrane protein 1, PhoT family | | CAETHG_3322 | CLJU_c12400 | CLRAG_11400 |
| 1377 | phosphate ABC transporter membrane protein 2, PhoT family | | CAETHG_3323 | CLJU_c12410 | CLRAG_11390 |
| 1378 | phosphate ABC transporter substrate-binding protein, PhoT family | | CAETHG_3321 | CLJU_c12390 | CLRAG_11410 |
| 1379 | phosphate:acyl-[acyl carrier protein] | 2.3.1.15 | CAETHG_3362 | CLJU_c12810 | CLRAG_11000 |
| | acyltransferase | | | | |
| 1380 | phosphatidylserine decarboxylase | 4.1.1.65 | CAETHG_2188 | CLJU_c00710 | CLRAG_19870 |
| 1381 | CDP-diacylglycerol---serine O- | 2.7.8.8 | CAETHG_2406 | CLJU_c02910 | CLRAG_28510 |
| | phosphatidyltransferase | | | | |
| 1382 | Phospho-N-acetylmuramoyl-pentapeptide- | 2.7.8.13 | CAETHG_3149 | CLJU_c10590 | CLRAG_12750 |
| | transferase | | | | |
| 1383 | phosphoenolpyruvate carboxykinase (ATP) | 4.1.1.49 | CAETHG_2721 | CLJU_c06210 | CLRAG_07490 |
| 1384 | phosphotransferase system, enzyme I, PtsI | 2.7.1.69 | CAETHG_1896 | CLJU_c40530 | CLRAG_22720 |
| 1385 | phosphoglucomutase | 5.4.2.10, | CAETHG_1320 | CLJU_c34210 | CLRAG_14360 |
| | | 5.4.2.2 | | | |
| 1386 | phosphoglucosamine mutase | 5.4.2.10, | CAETHG_1887 | CLJU_c40440 | CLRAG_22630 |
| | | 5.4.2.2 | | | |
| 1387 | D-3-phosphoglycerate dehydrogenase | 1.1.1.95 | CAETHG_1176 | CLJU_c32780 | CLRAG_15580 |
| 1388 | phosphoglycerate kinase | 2.7.2.3 | CAETHG_1759 | CLJU_c39140 | CLRAG_21290 |
| 1389 | phosphoglycerate mutase | 5.4.2.11 | CAETHG_1757 | CLJU_c39120 | CLRAG_21270 |
| 1390 | phosphoglucomutase | 5.4.2.8 | CAETHG_0898 | CLJU_c29060 | CLRAG_35060 |
| 1391 | hydroxymethylpyrimidine/phosphomethylpyrimidine | 2.7.1.49, | CAETHG_1202 | CLJU_c33040 | CLRAG_15290 |
| | kinase | 2.7.4.7 | | | |
| 1392 | Phosphopantetheine adenylyltransferase | 2.7.7.3 | CAETHG_3353 | CLJU_c12720 | CLRAG_11090 |
| 1393 | phosphopantothenoylcysteine decarboxylase/ | 4.1.1.36, | CAETHG_3336 | CLJU_c12540 | CLRAG_11260 |
| | phosphopantothenate--cysteine ligase | 6.3.2.5 | | | |
| 1394 | phosphopentomutase | 5.4.2.2, | CAETHG_3924 | CLJU_c18150 | CLRAG_00520 |
| | | 5.4.2.7 | | | |
| 1395 | phosphoribosyl-AMP cyclohydrolase | | CAETHG_3265 | CLJU_c11740 | CLRAG_11800 |
| 1396 | phosphoribosyl-ATP pyrophosphatase | 3.5.4.19, | CAETHG_3266 | CLJU_c11750 | CLRAG_11790 |
| | | 3.6.1.31 | | | |
| 1397 | phosphoribosylamine--glycine ligase | 6.3.4.13 | CAETHG_2954 | CLJU_c08600 | CLRAG_07890 |
| 1398 | phosphoribosylaminoimidazole- | 6.3.2.6 | CAETHG_2949 | CLJU_c08550 | CLRAG_07940 |
| | succinocarboxamide synthase | | | | |
| 1399 | phosphoribosylanthranilate isomerase | 5.3.1.24 | CAETHG_3705 | CLJU_c16110 | CLRAG_33080 |
| 1400 | phosphoribosylformylglycinamidine cyclo-ligase | 6.3.3.1 | CAETHG_2951 | CLJU_c08570 | CLRAG_07920 |
| 1401 | phosphoribosylformylglycinamidine synthase | 6.3.5.3 | CAETHG_3245 | CLJU_c11540 | CLRAG_11910 |
| 1402 | HAD-superfamily subfamily IB hydrolase, | 3.1.3.3 | CAETHG_3031 | CLJU_c09360 | CLRAG_13740 |
| | TIGR01490 | | | | |
| 1403 | phosphate acetyltransferase | 2.3.1.8 | CAETHG_3358 | CLJU_c12770 | CLRAG_11040 |
| 1404 | GH3 auxin-responsive promoter | | CAETHG_3993 | CLJU_c18740 | CLRAG_16550 |
| 1405 | Polyferredoxin | | CAETHG_0511 | CLJU_c24510 | CLRAG_30060 |
| 1406 | polyribonucleotide nucleotidyltransferase | | CAETHG_3404 | CLJU_c13210 | CLRAG_10580 |
| 1407 | porphobilinogen synthase | 4.2.1.24 | CAETHG_1124 | CLJU_c31960 | CLRAG_02590 |
| 1408 | positive regulator of sigma(E), RseC/MucC | | CAETHG_3226 | CLJU_c11350 | CLRAG_12100 |
| 1409 | K+-transporting ATPase ATPase A chain | | CAETHG_1801 | CLJU_c39560 | CLRAG_21760 |
| 1410 | K+-transporting ATPase ATPase B chain | | CAETHG_1800 | CLJU_c39550 | CLRAG_21750 |
| 1411 | K+-transporting ATPase ATPase C chain | | CAETHG_1799 | CLJU_c39540 | CLRAG_21740 |
| 1412 | precorrin-2/cobalt-factor-2 C20-methyltransferase | 2.1.1.151, | CAETHG_1117 | CLJU_c31890 | CLRAG_02520 |
| | | 2.1.1.130 | | | |
| 1413 | precorrin-2 dehydrogenase/sirohydrochlorin | 2.1.1.107, | CAETHG_1127 | CLJU_c31990 | CLRAG_02620 |
| | ferrochelatase | 1.3.1.76, | | | |
| | | 4.99.1.4 | | | |
| 1414 | precorrin-6Y C5,15-methyltransferase | 2.1.1.132 | CAETHG_1119 | CLJU_c31910 | CLRAG_02540 |
| | (decarboxylating) | | | | |
| 1415 | prephenate dehydrogenase | 1.3.1.52, | CAETHG_0909 | CLJU_c29170 | CLRAG_35170 |
| | | 1.3.1.13, | | | |
| | | 1.3.1.43, | | | |
| | | 1.3.1.12 | | | |
| 1416 | phosphatidylglycerol:prolipoprotein diacylglycerol | | CAETHG_2935 | CLJU_c08410 | CLRAG_08040 |
| | transferase | | | | |
| 1417 | prolyl-tRNA synthetase | | CAETHG_1694 | CLJU_c38360 | CLRAG_20830 |
| 1418 | ethanolamine utilization protein EutP | | CAETHG_3900 | CLJU_c17920 | CLRAG_00710 |
| 1419 | hypothetical protein | | CAETHG_0742 | CLJU_c26610 | CLRAG_08480 |
| 1420 | hypothetical protein | | CAETHG_2546 | CLJU_c04740 | CLRAG_38120 |
| 1421 | preprotein translocase subunit SecA | | CAETHG_2364 | CLJU_c02630 | CLRAG_28230 |
| 1422 | protein translocase subunit secY/sec61 alpha | | CAETHG_1927 | CLJU_c40840 | CLRAG_23030 |
| 1423 | protein-tyrosine phosphatase | | CAETHG_2335 | CLJU_c02300 | CLRAG_27900 |
| 1424 | PTS system, fructose-specific IIC component | 2.7.1.69 | CAETHG_0142 | CLJU_c20590 | CLRAG_19390 |
| 1425 | PTS system IIA component, Fru family | 2.7.1.69 | CAETHG_0676 | CLJU_c26070 | CLRAG_04160 |
| 1426 | PTS system, fructose-specific IIB-like component | | CAETHG_0674 | CLJU_c26050 | CLRAG_04140 |
| 1427 | PTS system, fructose-specific IIC-like component | 2.7.1.69 | CAETHG_0675 | CLJU_c26060 | CLRAG_04150 |
| 1428 | hypothetical protein | | CAETHG_2387 | CLJU_c02850, | CLRAG_28450 |
| | | | | CLJU_c15580 | |
| 1429 | purine-nucleoside phosphorylase | 2.4.2.23, | CAETHG_0160 | CLJU_c20750 | CLRAG_19250 |
| | | 2.4.2.2, | | | |
| | | 2.4.2.15, | | | |
| | | 2.4.2.4, | | | |
| | | 2.4.2.1 | | | |
| 1430 | purine operon repressor, PurR | | CAETHG_2009 | CLJU_c41800 | CLRAG_04930 |
| 1431 | CoA-substrate-specific enzyme activase, putative | | CAETHG_1482 | CLJU_c35740 | CLRAG_06270 |
| 1432 | 16S rRNA processing protein RimM | | CAETHG_3373 | CLJU_c12910 | CLRAG_10890 |
| 1433 | Benzoyl-CoA reductase/2-hydroxyglutaryl-CoA | | CAETHG_0196 | CLJU_c21100 | CLRAG_30680 |
| | dehydratase subunit, BcrC/BadD/HgdB | | | | |
| 1434 | Benzoyl-CoA reductase/2-hydroxyglutaryl-CoA | | CAETHG_1484 | CLJU_c35760 | CLRAG_06290 |
| | dehydratase subunit, BcrC/BadD/HgdB | | | | |
| 1435 | DNA-3-methyladenine glycosylase | | CAETHG_2298 | CLJU_c01950 | CLRAG_27550 |
| 1436 | small subunit ribosomal protein S1 | | CAETHG_0848 | CLJU_c28540 | CLRAG_34610 |
| 1437 | SSU ribosomal protein S21P | | CAETHG_2898 | CLJU_c08040 | CLRAG_08320 |
| 1438 | molecular chaperone Hsp33 | | CAETHG_1356 | CLJU_c34600 | CLRAG_14680 |
| 1439 | 4-hydroxybenzoate polyprenyltransferase | | CAETHG_3414 | CLJU_c13310 | CLRAG_10480 |
| 1440 | electron transport protein HydN | 1.12.1.4, | CAETHG_2797 | CLJU_c07060 | CLRAG_18910 |
| | | 1.1.99.33 | | | |
| 1441 | LSU ribosomal protein L21P | | CAETHG_2828 | CLJU_c07360 | CLRAG_26480 |
| 1442 | 8-oxo-dGTP diphosphatase | | CAETHG_3520, | CLJU_c14320 | CLRAG_09720 |
| | | | CAETHG_3545 | | |
| 1443 | N-glycosylase/DNA lyase | | CAETHG_1579 | CLJU_c37230 | CLRAG_36670 |
| 1444 | putative hydroxymethylpyrimidine transport | | CAETHG_0607 | CLJU_c25380 | CLRAG_03660 |
| | system substrate-binding protein | | | | |
| 1445 | lipoprotein-releasing system permease protein | | CAETHG_2657 | CLJU_c05650 | CLRAG_06870 |
| 1446 | iron complex transport system substrate-binding | | CAETHG_3822, | CLJU_c17250, | CLRAG_33970 |
| | protein | | CAETHG_3838 | CLJU_c17100 | |
| 1447 | iron complex transport system substrate-binding | | CAETHG_3827 | CLJU_c17150 | CLRAG_34010 |
| | protein | | | | |
| 1448 | iron complex transport system substrate-binding | | CAETHG_3833 | CLJU_c17200 | CLRAG_34200 |
| | protein | | | | |
| 1449 | ABC-2 type transport system ATP-binding protein | | CAETHG_0042, | CLJU_c18350, | CLRAG_39490 |
| | | | CAETHG_3942 | CLJU_c19650 | |
| 1450 | ABC-2 type transport system ATP-binding protein | | CAETHG_0354, | CLJU_c09140, | CLRAG_01870 |
| | | | CAETHG_3008 | CLJU_c37410, | |
| | | | | CLJU_c22920 | |
| 1451 | ABC-2 type transport system ATP-binding protein | | CAETHG_0388 | CLJU_c23250 | CLRAG_01480 |
| 1452 | ABC-2 type transport system ATP-binding protein | | CAETHG_0531 | CLJU_c24660 | CLRAG_18170 |
| 1453 | ABC-2 type transport system ATP-binding protein | | CAETHG_0580 | CLJU_c25120 | CLRAG_03460 |
| 1454 | putative hydroxymethylpyrimidine transport | | CAETHG_0609 | CLJU_c25400 | CLRAG_03680 |
| | system ATP-binding protein | | | | |
| 1455 | ABC-type multidrug transport system, ATPase | | CAETHG_0640 | CLJU_c25710 | CLRAG_03910 |
| | component | | | | |
| 1456 | putative ABC transport system ATP-binding protein | | CAETHG_0646 | CLJU_c25770 | CLRAG_04000 |
| 1457 | ABC-2 type transport system ATP-binding protein | | CAETHG_0658 | CLJU_c25890 | CLRAG_04030 |
| 1458 | putative spermidine/putrescine transport system | | CAETHG_0683 | CLJU_c26140 | CLRAG_04220 |
| | ATP-binding protein | | | | |
| 1459 | NitT/TauT family transport system ATP-binding | 3.A.1.17 | CAETHG_0732 | CLJU_c26510 | CLRAG_08390 |
| | protein | | | | |
| 1460 | Fe—S cluster assembly ATP-binding protein | | CAETHG_0775, | CLJU_c37940, | CLRAG_37330 |
| | | | CAETHG_1631 | CLJU_c26910 | |
| 1461 | ABC-2 type transport system ATP-binding protein | | CAETHG_0791 | CLJU_c27070 | CLRAG_08830 |
| 1462 | ABC-type multidrug transport system, ATPase | | CAETHG_0799 | CLJU_c27130 | CLRAG_20100 |
| | component | | | | |
| 1463 | putative ABC transport system ATP-binding protein | | CAETHG_0804, | CLJU_c01070, | CLRAG_20040 |
| | | | CAETHG_2218, | CLJU_c07680, | |
| | | | CAETHG_2870, | CLJU_c07780, | |
| | | | CAETHG_2861 | CLJU_c27190 | |
| 1464 | putative ABC transport system ATP-binding protein | | CAETHG_1016 | CLJU_c30160 | CLRAG_39150 |
| 1465 | ABC-2 type transport system ATP-binding protein | | CAETHG_1199 | CLJU_c33010 | CLRAG_15320 |
| 1466 | ABC-2 type transport system ATP-binding protein | | CAETHG_1427 | CLJU_c35190 | CLRAG_05700 |
| 1467 | ABC-2 type transport system ATP-binding protein | | CAETHG_1437 | CLJU_c35280 | CLRAG_05810 |
| 1468 | NitT/TauT family transport system ATP-binding | | CAETHG_1443 | CLJU_c35340 | CLRAG_05870 |
| | protein | | | | |
| 1469 | ATP-binding cassette, subfamily F, member 3 | | CAETHG_1582 | CLJU_c37260 | CLRAG_36700 |
| 1470 | ABC-2 type transport system ATP-binding protein | | CAETHG_1584 | CLJU_c37280 | CLRAG_36720 |
| 1471 | putative ABC transport system ATP-binding protein | | CAETHG_1713 | CLJU_c38630 | CLRAG_20950 |
| 1472 | putative ABC transport system ATP-binding protein | | CAETHG_1847, | CLJU_c40000, | CLRAG_22240 |
| | | | CAETHG_3515 | CLJU_c14420 | |
| 1473 | ATPase components of ABC transporters with | | CAETHG_1855 | CLJU_c40060 | CLRAG_22320 |
| | duplicated ATPase domains | | | | |
| 1474 | bacitracin transport system ATP-binding protein | | CAETHG_2195 | CLJU_c00800 | CLRAG_19780 |
| 1475 | ABC-2 type transport system ATP-binding protein | | CAETHG_2530, | CLJU_c04580, | CLRAG_37940 |
| | | | CAETHG_2868 | CLJU_c07760 | |
| 1476 | D-methionine transport system ATP-binding | | CAETHG_2532, | CLJU_c04600, | CLRAG_37960 |
| | protein | | CAETHG_2724 | CLJU_c06280 | |
| 1477 | lipoprotein-releasing system ATP-binding protein | | CAETHG_2658 | CLJU_c05660 | CLRAG_06880 |
| 1478 | bacitracin transport system ATP-binding protein | | CAETHG_2745 | CLJU_c06490 | CLRAG_30410 |
| 1479 | ABC-type nitrate/sulfonate/bicarbonate transport | | CAETHG_2976 | CLJU_c08810 | CLRAG_07680 |
| | system, ATPase component | | | | |
| 1480 | ABC-2 type transport system ATP-binding protein | | CAETHG_3462 | CLJU_c13790 | CLRAG_10050 |
| 1481 | putative ABC transport system ATP-binding protein | | CAETHG_3506 | CLJU_c14240 | CLRAG_09560 |
| 1482 | putative ABC transport system ATP-binding protein | | CAETHG_3512 | CLJU_c14300 | CLRAG_09660 |
| 1483 | putative ABC transport system ATP-binding protein | | CAETHG_3650, | CLJU_c15490, | CLRAG_00560 |
| | | | CAETHG_3918 | CLJU_c18090 | |
| 1484 | iron complex transport system ATP-binding | | CAETHG_3828 | CLJU_c17160 | CLRAG_34020 |
| | protein | | | | |
| 1485 | macrolide transport system ATP-binding/permease | | CAETHG_4036 | CLJU_c19020 | CLRAG_40020 |
| | protein | | | | |
| 1486 | ATP-binding cassette, subfamily B | | CAETHG_3620 | CLJU_c15180 | CLRAG_24170 |
| 1487 | ATP-binding cassette, subfamily B | | CAETHG_4025 | CLJU_c18910 | CLRAG_40060 |
| 1488 | ABC-2 type transport system permease protein | | CAETHG_0530 | CLJU_c24650 | CLRAG_18180 |
| 1489 | putative hydroxymethylpyrimidine transport | | CAETHG_0608 | CLJU_c25390 | CLRAG_03670 |
| | system permease protein | | | | |
| 1490 | putative spermidine/putrescine transport system | | CAETHG_0684 | CLJU_c26150 | CLRAG_04230 |
| | permease protein | | | | |
| 1491 | putative spermidine/putrescine transport system | | CAETHG_0685 | CLJU_c26160 | CLRAG_04240 |
| | permease protein | | | | |
| 1492 | NitT/TauT family transport system permease | 3.A.1.17 | CAETHG_0733 | CLJU_c26520 | CLRAG_08400 |
| | protein | | | | |
| 1493 | NitT/TauT family transport system permease | | CAETHG_1444 | CLJU_c35350 | CLRAG_05880 |
| | protein | | | | |
| 1494 | putative ABC transport system permease protein | | CAETHG_1846 | CLJU_c39990 | CLRAG_22230 |
| 1495 | bacitracin transport system permease protein | | CAETHG_2194 | CLJU_c00790 | CLRAG_19790 |
| 1496 | putative ABC transport system permease protein | | CAETHG_2219 | CLJU_c01080 | CLRAG_30320 |
| 1497 | bacitracin transport system permease protein | | CAETHG_2744 | CLJU_c06480 | CLRAG_30420 |
| 1498 | putative ABC transport system permease protein | | CAETHG_2862 | CLJU_c07690 | CLRAG_25220 |
| 1499 | hypothetical protein | | CAETHG_3464 | CLJU_c13810 | CLRAG_10030 |
| 1500 | putative ABC transport system permease protein | | CAETHG_3507 | CLJU_c14250 | CLRAG_09570 |
| 1501 | iron complex transport system permease protein | | CAETHG_3825 | CLJU_c17130 | CLRAG_33990 |
| 1502 | putative ABC transport system permease protein | | CAETHG_3919 | CLJU_c18100 | CLRAG_00550 |
| 1503 | ABC-2 type transport system permease protein | | CAETHG_3943 | CLJU_c18360 | CLRAG_00310 |
| 1504 | putative spermidine/putrescine transport system | | CAETHG_0682 | CLJU_c26130 | CLRAG_04210 |
| | substrate-binding protein | | | | |
| 1505 | ATP-binding cassette, subfamily B | | CAETHG_4024 | CLJU_c18900 | CLRAG_40070 |
| 1506 | ABC-2 type transport system permease protein | | CAETHG_0043 | CLJU_c19660 | CLRAG_39480 |
| 1507 | acetaldehyde dehydrogenase (acetylating) | | CAETHG_1830 | CLJU_c39840 | CLRAG_22090 |
| 1508 | acetyl-CoA C-acetyltransferase | | CAETHG_0427 | CLJU_c23630 | CLRAG_17540 |
| 1509 | Acetyltransferase (GNAT) family protein | | CAETHG_0216 | CLJU_c21300 | CLRAG_30860 |
| 1510 | Acetyltransferase (GNAT) domain-containing | | CAETHG_0923, | CLJU_c29300, | CLRAG_35310 |
| | protein | | CAETHG_3174 | CLJU_c10850 | |
| 1511 | phosphinothricin acetyltransferase | | CAETHG_1060 | CLJU_c30550 | CLRAG_08730 |
| 1512 | Ribosomal protein S18 acetylase RimI | | CAETHG_1413 | CLJU_c35040 | CLRAG_26300 |
| 1513 | N-acetylglutamate synthase, GNAT family | | CAETHG_1749 | CLJU_c39010 | CLRAG_21190 |
| 1514 | Ribosomal protein S18 acetylase RimI | | CAETHG_2735 | CLJU_c06400 | CLRAG_30450 |
| 1515 | Predicted nucleotide-binding protein, sugar | | CAETHG_0183 | CLJU_c20990 | CLRAG_19030 |
| | kinase/HSP70/actin superfamily | | | | |
| 1516 | CoA-substrate-specific enzyme activase, putative | | CAETHG_1442 | CLJU_c35330 | CLRAG_05860 |
| 1517 | acyl carrier protein | | CAETHG_3363 | CLJU_c12820 | CLRAG_10990 |
| 1518 | medium-chain acyl-[acyl-carrier-protein] hydrolase | | CAETHG_2058 | CLJU_c42340 | CLRAG_05450 |
| 1519 | Acyl-CoA dehydrogenase | 1.3.1.44, | CAETHG_1787 | CLJU_c39420 | CLRAG_21630 |
| | | 1.3.99.2 | | | |
| 1520 | acyl-CoA dehydrogenase | 1.3.1.44, | CAETHG_1789 | CLJU_c39440 | CLRAG_21650 |
| | | 1.3.99.2 | | | |
| 1521 | Crotonobetainyl-CoA:carnitine CoA-transferase | | CAETHG_1788 | CLJU_c39430 | CLRAG_21640 |
| | CaiB | | | | |
| 1522 | Surface polysaccharide O-acyltransferase, integral | | CAETHG_1301 | CLJU_c34030 | CLRAG_14180 |
| | membrane enzyme | | | | |
| 1523 | Fucose 4-O-acetylase | | CAETHG_1311 | CLJU_c34130 | CLRAG_14280 |
| 1524 | adenylate cyclase, class 2 | 4.6.1.1 | CAETHG_2381 | CLJU_c02800 | CLRAG_28400 |
| 1525 | ADP-ribosyl-[dinitrogen reductase] hydrolase | | CAETHG_0078 | CLJU_c19980 | CLRAG_39060 |
| 1526 | hypothetical protein | | CAETHG_0588 | CLJU_c25200 | CLRAG_03550 |
| 1527 | Aldo/keto reductase | | CAETHG_0821 | CLJU_c28210 | CLRAG_09160 |
| 1528 | aryl-alcohol dehydrogenase (NADP+) | | CAETHG_3819 | CLJU_c17070 | CLRAG_33940 |
| 1529 | Aldo/keto reductase | 1.1.1.21 | CAETHG_3890 | CLJU_c17820 | CLRAG_00890 |
| 1530 | Uncharacterized conserved protein YloU, alkaline | | CAETHG_3200 | CLJU_c11110 | CLRAG_12350 |
| | shock protein (Asp23) family | | | | |
| 1531 | Uncharacterized conserved protein YloU, alkaline | | CAETHG_3349 | CLJU_c12680 | CLRAG_11130 |
| | shock protein (Asp23) family | | | | |
| 1532 | D-lactate dehydrogenase | 1.1.1.28 | CAETHG_1147 | CLJU_c32190 | CLRAG_02820 |
| 1533 | Pimeloyl-ACP methyl ester carboxylesterase | | CAETHG_3573, | CLJU_c17490, | CLRAG_20450 |
| | | | CAETHG_3862 | CLJU_c14740 | |
| 1534 | Cystathionine beta-lyase family protein involved in | | CAETHG_0206 | CLJU_c21200 | CLRAG_30780 |
| | aluminum resistance | | | | |
| 1535 | putative selenium metabolism protein SsnA | | CAETHG_0447 | CLJU_c23820 | CLRAG_17350 |
| 1536 | Cytosine/adenosine deaminase | | CAETHG_0680 | CLJU_c26110 | CLRAG_04190 |
| 1537 | Cytosine/adenosine deaminase | 3.5.4.3 | CAETHG_1058 | CLJU_c30530 | CLRAG_15950 |
| 1538 | amidohydrolase | | CAETHG_1246 | CLJU_c33470 | CLRAG_32300 |
| 1539 | amidohydrolase | 3.5.1.47 | CAETHG_3847 | CLJU_c17340 | CLRAG_29240 |
| 1540 | competence protein ComFC | | CAETHG_2362 | CLJU_c02610 | CLRAG_28210 |
| 1541 | L-asparagine transporter | | CAETHG_2486 | CLJU_c04180 | CLRAG_26880 |
| 1542 | Amino acid transporter | | CAETHG_1744 | CLJU_c38960 | CLRAG_21140 |
| 1543 | L-asparagine transporter | | CAETHG_1909 | CLJU_c40660, | CLRAG_25010 |
| | | | | CLJU_c16020 | |
| 1544 | Threonine/homoserine/homoserine lactone efflux | | CAETHG_4019 | CLJU_c18870 | CLRAG_40130 |
| | protein | | | | |
| 1545 | Putative aminopeptidase FrvX | | CAETHG_3608 | CLJU_c15060 | CLRAG_24310 |
| 1546 | Aspartyl aminopeptidase | | CAETHG_0278 | CLJU_c21880 | CLRAG_31420 |
| 1547 | Aspartate/methionine/tyrosine aminotransferase | 2.6.1.23, | CAETHG_0933 | CLJU_c29390 | CLRAG_35390 |
| | | 2.6.1.1 | | | |
| 1548 | aminotransferase | 2.6.1.— | CAETHG_1350 | CLJU_c34540 | CLRAG_14620 |
| 1549 | taurine---2-oxoglutarate transaminase | 2.6.1.62 | CAETHG_1499 | CLJU_c35920 | CLRAG_06420 |
| 1550 | aspartate aminotransferase | 2.6.1.51, | CAETHG_2210 | CLJU_c00960 | CLRAG_19620 |
| | | 2.6.1.44 | | | |
| 1551 | aspartate aminotransferase | 2.6.1.51, | CAETHG_2224 | CLJU_c01130 | CLRAG_30270 |
| | | 2.6.1.44 | | | |
| 1552 | Phenylacetate-coenzyme A ligase PaaK, adenylate- | | CAETHG_0467 | CLJU_c24010 | CLRAG_17150 |
| | forming domain family | | | | |
| 1553 | acyl-CoA synthetase | 6.2.1.3 | CAETHG_1784 | CLJU_c39390 | CLRAG_21600 |
| 1554 | anaerobic ribonucleoside-triphosphate reductase | | CAETHG_2288 | CLJU_c01850 | CLRAG_27450 |
| | activating protein | | | | |
| 1555 | stage II sporulation protein AB (anti-sigma F factor) | | CAETHG_1294 | CLJU_c33960 | CLRAG_14110 |
| 1556 | AraC-type DNA-binding protein | | CAETHG_0064 | CLJU_c19840 | CLRAG_39240 |
| 1557 | Arginine utilization protein RocB | | CAETHG_0384 | CLJU_c23210 | CLRAG_01520 |
| 1558 | arginine decarboxylase | 4.1.1.18, | CAETHG_1321 | CLJU_c34220 | CLRAG_14370 |
| | | 4.1.1.19, | | | |
| | | 4.1.1.17 | | | |
| 1559 | aspartate aminotransferase | 2.6.1.9, | CAETHG_2537 | CLJU_c04650 | CLRAG_38030 |
| | | 2.6.1.58, | | | |
| | | 2.6.1.1 or | | | |
| | | 2.6.1.9, | | | |
| | | 2.6.1.57, | | | |
| | | 2.6.1.23, | | | |
| | | 2.6.1.78, | | | |
| | | 2.6.1.5, | | | |
| | | 2.6.1.1 | | | |
| 1560 | Arsenical resistance operon trans-acting repressor | | CAETHG_3664 | CLJU_c15650 | CLRAG_32710 |
| | ArsD | | | | |
| 1561 | Predicted ATPase | | CAETHG_0931 | CLJU_c29370 | CLRAG_33860 |
| 1562 | DNA helicase-2/ATP-dependent DNA helicase | | CAETHG_1692 | CLJU_c38340 | CLRAG_20810 |
| | PcrA | | | | |
| 1563 | DNA helicase-2/ATP-dependent DNA helicase | | CAETHG_3749 | CLJU_c16530 | CLRAG_33340 |
| | PcrA | | | | |
| 1564 | cell division protease FtsH | | CAETHG_1693 | CLJU_c38350 | CLRAG_20820 |
| 1565 | Superfamily II DNA and RNA helicase | | CAETHG_0361 | CLJU_c22990 | CLRAG_01790 |
| 1566 | Superfamily II DNA and RNA helicase | | CAETHG_3006 | CLJU_c09120 | CLRAG_13900 |
| 1567 | ATP-dependent RNA helicase SUPV3L1/SUV3 | | CAETHG_4041 | CLJU_c19070 | CLRAG_39950 |
| 1568 | hypothetical protein | | CAETHG_1975 | CLJU_c41340 | CLRAG_23530 |
| 1569 | hypothetical protein | | CAETHG_2055 | CLJU_c42310 | CLRAG_05420 |
| 1570 | tRNA threonylcarbamoyladenosine biosynthesis | | CAETHG_1674 | CLJU_c38180 | CLRAG_20640 |
| | protein TsaE | | | | |
| 1571 | ATP-dependent Clp protease ATP-binding subunit | | CAETHG_1974 | CLJU_c41330 | CLRAG_23520 |
| | ClpC | | | | |
| 1572 | cell division transport system ATP-binding protein | | CAETHG_2422 | CLJU_c03070 | CLRAG_28680 |
| 1573 | tRNA(Ile)-lysidine synthase TilS/MesJ | | CAETHG_2551 | CLJU_c04790 | CLRAG_38170 |
| 1574 | ATP-binding cassette, subfamily F, uup | | CAETHG_2933 | CLJU_c08390 | CLRAG_08060 |
| 1575 | magnesium chelatase family protein | | CAETHG_3381 | CLJU_c12980 | CLRAG_10810 |
| 1576 | penicillin-binding protein | | CAETHG_2700 | CLJU_c06020 | CLRAG_07250 |
| 1577 | beta-lactamase class A | | CAETHG_3737 | CLJU_c16430 | CLRAG_33230 |
| 1578 | penicillin-binding protein 1A | | CAETHG_1292 | CLJU_c33940 | CLRAG_14090 |
| 1579 | biotin transport system substrate-specific | | CAETHG_0507 | CLJU_c24470 | CLRAG_30100 |
| | component | | | | |
| 1580 | acetyl-CoA carboxylase biotin carboxyl carrier | 6.3.4.14 | CAETHG_2044 | CLJU_c42140 | CLRAG_05250 |
| | protein | | | | |
| 1581 | biotin synthase | | CAETHG_1691 | CLJU_c38330 | CLRAG_20800 |
| 1582 | Biotin-requiring enzyme | | CAETHG_0132 | CLJU_c20500 | CLRAG_19520 |
| 1583 | 4-azaleucine resistance probable transporter AzlC | | CAETHG_3451 | CLJU_c13690 | CLRAG_10180 |
| 1584 | cation:H+ antiporter | | CAETHG_2476 | CLJU_c04130 | CLRAG_26920 |
| 1585 | cAMP-binding domain of CRP or a regulatory | | CAETHG_3496 | CLJU_c14150 | CLRAG_09470 |
| | subunit of cAMP-dependent protein kinases | | | | |
| 1586 | NDP-sugar epimerase, includes UDP-GlcNAc- | | CAETHG_1317 | CLJU_c34180 | CLRAG_14330 |
| | inverting 4,6-dehydratase FlaA1 and capsular | | | | |
| | polysaccharide biosynthesis protein EpsC | | | | |
| 1587 | Capsular polysaccharide biosynthesis protein | | CAETHG_2591 | CLJU_c05140 | CLRAG_38530 |
| 1588 | protein-tyrosine phosphatase | | CAETHG_2592 | CLJU_c05150 | CLRAG_38540 |
| 1589 | xylulokinase | 2.7.1.17 | CAETHG_3597 | CLJU_c14910 | CLRAG_20240 |
| 1590 | cardiolipin synthase | | CAETHG_0896 | CLJU_c29040 | CLRAG_35040 |
| 1591 | cardiolipin synthase | | CAETHG_2984 | CLJU_c08890 | CLRAG_07590 |
| 1592 | cation diffusion facilitator family transporter | | CAETHG_0534 | CLJU_c24690 | CLRAG_18140 |
| 1593 | glycoside/pentoside/hexuronide:cation symporter, | | CAETHG_2234 | CLJU_c01250 | CLRAG_30170 |
| | GPH family | | | | |
| 1594 | Cd2+/Zn2+-exporting ATPase | | CAETHG_0899 | CLJU_c29070 | CLRAG_35070 |
| 1595 | Ca2+-transporting ATPase | | CAETHG_2779 | CLJU_c06890 | CLRAG_18720 |
| 1596 | ATPase, P-type (transporting), HAD superfamily, | | CAETHG_2880 | CLJU_c07870 | CLRAG_25350 |
| | subfamily IC | | | | |
| 1597 | Ca2+-transporting ATPase | | CAETHG_3138 | CLJU_c10480 | CLRAG_12890 |
| 1598 | hydrophobic/amphiphilic exporter-1, HAE1 family | | CAETHG_0391 | CLJU_c23280 | CLRAG_01450 |
| 1599 | CDP-diacylglycerol--glycerol-3-phosphate 3- | 2.7.8.5 | CAETHG_1386 | CLJU_c34880 | CLRAG_26120 |
| | phosphatidyltransferase | | | | |
| 1600 | rod shape determining protein RodA | | CAETHG_3814 | CLJU_c17020 | CLRAG_33900 |
| 1601 | stage V sporulation protein D (sporulation-specific | | CAETHG_1729, | CLJU_c10560, | CLRAG_21070 |
| | penicillin-binding protein) | | CAETHG_3146 | CLJU_c38810 | |
| 1602 | cell division protein FtsW, lipid II flippase | | CAETHG_2429 | CLJU_c03140 | CLRAG_28750 |
| 1603 | cell division protein FtsW | | CAETHG_3150 | CLJU_c10600 | CLRAG_12740 |
| 1604 | cell division protease FtsH | | CAETHG_2710 | CLJU_c06110 | CLRAG_07390 |
| 1605 | lysozyme | | CAETHG_1001 | CLJU_c30020 | CLRAG_15660 |
| 1606 | N-acetylmuramoyl-L-alanine amidase | | CAETHG_1891 | CLJU_c40480 | CLRAG_22670 |
| 1607 | Beta- N-acetylglucosaminidase | | CAETHG_2267 | CLJU_c01670 | CLRAG_27270 |
| 1608 | LysM domain-containing protein | | CAETHG_2538 | CLJU_c04660 | CLRAG_38040 |
| 1609 | N-acetylmuramoyl-L-alanine amidase | | CAETHG_2554 | CLJU_c04820 | CLRAG_38200 |
| 1610 | Putative cell wall binding repeat 2 | | CAETHG_0540 | CLJU_c24750 | CLRAG_18070 |
| 1611 | Putative cell wall binding repeat 2 | | CAETHG_0856 | CLJU_c28610 | CLRAG_34690 |
| 1612 | pilus assembly protein CpaB | | CAETHG_0879 | CLJU_c28830 | CLRAG_34910 |
| 1613 | Putative cell wall binding repeat 2 | | CAETHG_0988 | CLJU_c29890 | CLRAG_17030 |
| 1614 | Putative cell wall-binding protein | | CAETHG_1543, | CLJU_c36350, | CLRAG_38390 |
| | | | CAETHG_2577 | CLJU_c05000 | |
| 1615 | Putative cell wall binding repeat 2 | | CAETHG_2557 | CLJU_c04840 | CLRAG_38220 |
| 1616 | Putative cell wall binding repeat 2 | | CAETHG_2588 | CLJU_c05110 | CLRAG_38500 |
| 1617 | Putative cell wall binding repeat 2 | | CAETHG_2589 | CLJU_c05120 | CLRAG_38510 |
| 1618 | Putative cell wall-binding protein | | CAETHG_2655, | CLJU_c18600, | CLRAG_00090 |
| | | | CAETHG_3968 | CLJU_c05630, | |
| | | | | CLJU_c32530 | |
| 1619 | Putative cell wall-binding protein | | CAETHG_2960 | CLJU_c08660 | CLRAG_07830 |
| 1620 | Putative cell wall-binding protein | | CAETHG_3980 | CLJU_c18660 | CLRAG_16980 |
| 1621 | cell division initiation protein | | CAETHG_3159 | CLJU_c10690 | CLRAG_12650 |
| 1622 | molecular chaperone DnaJ | | CAETHG_2892 | CLJU_c07990 | CLRAG_25470 |
| 1623 | chemotaxis protein CheX | | CAETHG_4018 | CLJU_c18860 | CLRAG_40140 |
| 1624 | two-component system, chemotaxis family, sensor | | CAETHG_0310 | CLJU_c22120 | CLRAG_31700 |
| | kinase CheA | | | | |
| 1625 | purine-binding chemotaxis protein CheW | | CAETHG_0309 | CLJU_c22110 | CLRAG_31690 |
| 1626 | two-component system, chemotaxis family, | | CAETHG_0311, | CLJU_c22130, | CLRAG_31710 |
| | response regulator CheY | | CAETHG_3040 | CLJU_c09450 | |
| 1627 | Methyl-accepting chemotaxis protein (MCP) | | CAETHG_1064 | CLJU_c30600 | CLRAG_16010 |
| | signalling domain-containing protein | | | | |
| 1628 | Thiamine kinase | | CAETHG_1812 | CLJU_c39660 | CLRAG_21910 |
| 1629 | chromate transporter | | CAETHG_1526 | CLJU_c36160 | CLRAG_24020 |
| 1630 | chromate transporter | | CAETHG_3865 | CLJU_c17570 | CLRAG_01170 |
| 1631 | chromosome partitioning protein | | CAETHG_2114 | CLJU_c42880 | CLRAG_25690 |
| 1632 | chromosome partitioning protein, ParB family | | CAETHG_2115 | CLJU_c42890 | CLRAG_25700 |
| 1633 | 2-aminoadipate transaminase | | CAETHG_0036, | CLJU_c21270, | CLRAG_39530 |
| | | | CAETHG_0213 | CLJU_c19590 | |
| 1634 | CoA-substrate-specific enzyme activase, putative | | CAETHG_0182 | CLJU_c20980 | CLRAG_19040 |
| 1635 | methylmalonyl-CoA mutase C-terminal domain- | | CAETHG_0193, | CLJU_c21080, | CLRAG_30660 |
| | containing protein/methyltransferase cognate | | CAETHG_4045 | CLJU_c19110 | |
| | corrinoid proteins | | | | |
| 1636 | cobalt/nickel transport system ATP-binding protein | | CAETHG_0854 | CLJU_c28590 | CLRAG_34670 |
| 1637 | cobalt/nickel transport system ATP-binding protein | | CAETHG_1131 | CLJU_c32030 | CLRAG_02660 |
| 1638 | energy-coupling factor transport system ATP- | | CAETHG_1917 | CLJU_c40740 | CLRAG_22930 |
| | binding protein | | | | |
| 1639 | energy-coupling factor transport system ATP- | | CAETHG_1918 | CLJU_c40750 | CLRAG_22940 |
| | binding protein | | | | |
| 1640 | energy-coupling factor transport system permease | | CAETHG_1916 | CLJU_c40730 | CLRAG_22920 |
| | protein | | | | |
| 1641 | cobalt/nickel transport system permease protein | | CAETHG_1132 | CLJU_c32040 | CLRAG_02670 |
| 1642 | cobalt/nickel transport system permease protein | | CAETHG_0321 | CLJU_c22230 | CLRAG_31810 |
| 1643 | cobalt/nickel transport system permease protein | | CAETHG_1219 | CLJU_c33200 | CLRAG_15060 |
| 1644 | cobalt/nickel transport system permease protein | | CAETHG_0855 | CLJU_c28600 | CLRAG_34680 |
| 1645 | hypothetical protein | | CAETHG_2719 | CLJU_c06190 | CLRAG_07470 |
| 1646 | CO dehydrogenase maturation factor | | CAETHG_1612 | CLJU_c37590 | CLRAG_37000 |
| 1647 | CO dehydrogenase maturation factor | | CAETHG_1619 | CLJU_c37660 | CLRAG_37070 |
| 1648 | cold shock protein (beta-ribbon, CspA family) | | CAETHG_1223 | CLJU_c33240 | CLRAG_15020 |
| 1649 | competence protein ComEC | | CAETHG_2749 | CLJU_c06590 | CLRAG_18400 |
| 1650 | CopG family transcriptional regulator/antitoxin | | CAETHG_2418 | CLJU_c03030 | CLRAG_28640 |
| | EndoAI | | | | |
| 1651 | methylmalonyl-CoA mutase C-terminal domain- | | CAETHG_0139, | CLJU_c20700, | CLRAG_30740 |
| | containing protein | | CAETHG_0154, | CLJU_c20670, | |
| | | | CAETHG_0150 | CLJU_c20560 | |
| 1652 | Methanogenic corrinoid protein MtbC1 | | CAETHG_0159 | CLJU_c20740 | CLRAG_19260 |
| 1653 | Uroporphyrinogen decarboxylase (URO-D) | | CAETHG_2845, | CLJU_c07570, | CLRAG_32350 |
| | | | CAETHG_2850 | CLJU_c07520 | |
| 1654 | CrcB protein | | CAETHG_0397 | CLJU_c23330 | CLRAG_01410 |
| 1655 | CrcB protein | | CAETHG_0398 | CLJU_c23340 | CLRAG_01400 |
| 1656 | cAMP-binding domain of CRP or a regulatory | | CAETHG_0443 | CLJU_c23780 | CLRAG_17390 |
| | subunit of cAMP-dependent protein kinases | | | | |
| 1657 | CRP/FNR family transcriptional regulator, | | CAETHG_3172 | CLJU_c10830 | CLRAG_03130 |
| | anaerobic regulatory protein | | | | |
| 1658 | MFS transporter, CP family, cyanate transporter | | CAETHG_0544 | CLJU_c24790 | CLRAG_18000 |
| 1659 | Kynurenine formamidase | | CAETHG_0429 | CLJU_c23650 | CLRAG_17520 |
| 1660 | Kynurenine formamidase | | CAETHG_0484, | CLJU_c08710, | CLRAG_24930 |
| | | | CAETHG_2965 | CLJU_c24260 | |
| 1661 | D-3-phosphoglycerate dehydrogenase | 1.1.1.95 | CAETHG_1065 | CLJU_c30610 | CLRAG_16020 |
| 1662 | D-tyrosyl-tRNA(Tyr) deacylase | | CAETHG_1268 | CLJU_c33700 | CLRAG_24580 |
| 1663 | putative selenium metabolism hydrolase | | CAETHG_0446 | CLJU_c23810 | CLRAG_17360 |
| 1664 | polysaccharide deacetylase family sporulation | | CAETHG_1360 | CLJU_c34640 | CLRAG_14720 |
| | protein PdaB | | | | |
| 1665 | EDD domain protein, DegV family | | CAETHG_1763 | CLJU_c39180 | CLRAG_21330 |
| 1666 | putative dehydratase, YjhG/YagF family | 4.2.1.9 | CAETHG_2179 | CLJU_c00610 | CLRAG_19970 |
| 1667 | NAD(P)-dependent dehydrogenase, short-chain | 1.1.1.0, | CAETHG_0079, | CLJU_c19990, | CLRAG_39050 |
| | alcohol dehydrogenase family | 2.3.1.86, | CAETHG_0982 | CLJU_c29830 | |
| | | 1.1.1.100, | | | |
| | | 1.1.1.1, | | | |
| | | 2.3.1.85 | | | |
| 1668 | Predicted dehydrogenase | | CAETHG_0673 | CLJU_c26040 | CLRAG_04130 |
| 1669 | NAD(P)-dependent dehydrogenase, short-chain | 2.3.1.85, | CAETHG_1743 | CLJU_c38950 | CLRAG_21130 |
| | alcohol dehydrogenase family | 2.3.1.86, | | | |
| | | 1.1.1.1, | | | |
| | | 1.1.1.100, | | | |
| | | 1.1.1.0 | | | |
| 1670 | hypothetical protein | | CAETHG_2060 | CLJU_c42360 | CLRAG_05470 |
| 1671 | dGTPase | 3.1.5.1 | CAETHG_2475 | CLJU_c04120 | CLRAG_26930 |
| 1672 | TatD DNase family protein | | CAETHG_2276 | CLJU_c01740 | CLRAG_27340 |
| 1673 | 4-hydroxy-tetrahydrodipicolinate synthase | 4.2.1.52 | CAETHG_2446 | CLJU_c03840 | CLRAG_28910 |
| 1674 | Predicted Fe—Mo cluster-binding protein, NifX | | CAETHG_1623 | CLJU_c37690 | CLRAG_37110 |
| | family | | | | |
| 1675 | cation diffusion facilitator family transporter | | CAETHG_1456 | CLJU_c35480 | CLRAG_06010 |
| 1676 | NRAMP (natural resistance-associated macrophage protein) metal | | CAETHG_2064 | CLJU_c42390 | CLRAG_05530 |
| | ion transporters | | | | |
| 1677 | CBS domain-containing protein | | CAETHG_3696 | CLJU_c15990 | CLRAG_32980 |
| 1678 | NRAMP (natural resistance-associated macrophage protein) metal | | CAETHG_3697 | CLJU_c16000 | CLRAG_32990 |
| | ion transporters | | | | |
| 1679 | Uncharacterized conserved protein YlxW, UPF0749 | | CAETHG_3152 | CLJU_c10620 | CLRAG_12720 |
| | family | | | | |
| 1680 | Uncharacterized conserved protein YlxW, UPF0749 | | CAETHG_3154 | CLJU_c10640 | CLRAG_12700 |
| | family | | | | |
| 1681 | replicative DNA helicase | | CAETHG_2096 | CLJU_c42710 | CLRAG_25520 |
| 1682 | prepilin-type N-terminal cleavage/methylation | | CAETHG_3183 | CLJU_c10940 | CLRAG_12520 |
| | domain-containing protein | | | | |
| 1683 | MutS domain III | | CAETHG_0272 | CLJU_c21820 | CLRAG_31360 |
| 1684 | MutS domain V | | CAETHG_0316 | CLJU_c22180 | CLRAG_31760 |
| 1685 | adenine-specific DNA-methyltransferase | | CAETHG_1296 | CLJU_c33980 | CLRAG_14130 |
| 1686 | DNA replication protein DnaC | | CAETHG_2054 | CLJU_c42230 | CLRAG_05340 |
| 1687 | DnaD and phage-associated domain-containing | | CAETHG_2053 | CLJU_c42220 | CLRAG_05330 |
| | protein | | | | |
| 1688 | competence protein ComEA | | CAETHG_2837 | CLJU_c07450 | CLRAG_32240 |
| 1689 | putative DNA modification/repair radical SAM | | CAETHG_1034 | CLJU_c30270 | CLRAG_15910 |
| | protein | | | | |
| 1690 | Predicted DNA-binding protein, UPF0251 family | | CAETHG_1624 | CLJU_c37700 | CLRAG_37120 |
| 1691 | putative transcriptional regulator | | CAETHG_1667 | CLJU_c32500 | CLRAG_36110 |
| 1692 | diadenylate cyclase | | CAETHG_1972 | CLJU_c41310 | CLRAG_23500 |
| 1693 | electron transport complex, RnfABCDGE type, B | 1.18.1.3 | CAETHG_3232 | CLJU_c11410 | CLRAG_12040 |
| | subunit | | | | |
| 1694 | electron transport complex protein RnfC | 1.18.1.3 | CAETHG_3227 | CLJU_c11360 | CLRAG_12090 |
| 1695 | electron transport complex protein RnfG | 1.18.1.3 | CAETHG_3229 | CLJU_c11380 | CLRAG_12070 |
| 1696 | small GTP-binding protein domain-containing | | CAETHG_0529 | CLJU_c24640 | CLRAG_18260 |
| | protein | | | | |
| 1697 | Endodeoxyribonuclease RusA | | CAETHG_1596 | CLJU_c37440 | CLRAG_36850 |
| 1698 | hypothetical protein | | CAETHG_2823 | CLJU_c07310 | CLRAG_26530 |
| 1699 | phenazine biosynthesis protein PhzF family | | CAETHG_0019 | CLJU_c19420 | CLRAG_39690 |
| 1700 | phenazine biosynthesis protein PhzF family | | CAETHG_3885 | CLJU_c17770 | CLRAG_00940 |
| 1701 | erythromycin esterase | | CAETHG_0637 | CLJU_c25680 | CLRAG_03880 |
| 1702 | NTE family protein | | CAETHG_0169 | CLJU_c20840 | CLRAG_19160 |
| 1703 | hypothetical protein | | CAETHG_1027 | CLJU_c30200 | CLRAG_15800 |
| 1704 | ethanolamine utilization protein EutJ | | CAETHG_1826, | CLJU_c39800, | CLRAG_22050 |
| | | | CAETHG_3282 | CLJU_c11910 | |
| 1705 | ethanolamine utilization protein EutQ | | CAETHG_1821, | CLJU_c39750, | CLRAG_22000 |
| | | | CAETHG_3285 | CLJU_c11940 | |
| 1706 | excinuclease ABC subunit A | | CAETHG_3574 | CLJU_c14750 | CLRAG_20400 |
| 1707 | DNA polymerase-3 subunit epsilon | | CAETHG_4048 | CLJU_c19140 | CLRAG_39940 |
| 1708 | DNA polymerase-3 subunit epsilon | | CAETHG_1533 | CLJU_c36230 | CLRAG_23950 |
| 1709 | Exopolysaccharide biosynthesis protein | | CAETHG_0968 | CLJU_c29700, | CLRAG_35700 |
| | | | | CLJU_c15840 | |
| 1710 | capsular exopolysaccharide family | | CAETHG_2593 | CLJU_c05160 | CLRAG_38550 |
| 1711 | Exopolysaccharide biosynthesis protein | | CAETHG_2955 | CLJU_c08610 | CLRAG_07880 |
| 1712 | hypothetical protein | | CAETHG_1888 | CLJU_c40450 | CLRAG_22640 |
| 1713 | Dehydrogenase (flavoprotein) | | CAETHG_0813 | CLJU_c27270 | CLRAG_08970 |
| 1714 | NADPH-dependent 2,4-dienoyl-CoA reductase, | | CAETHG_0943 | CLJU_c29490 | CLRAG_35480 |
| | sulfur reductase | | | | |
| 1715 | glycolate oxidase | 1.1.3.15 | CAETHG_0117, | CLJU_c05910, | CLRAG_25940 |
| | | | CAETHG_2688, | CLJU_c23190, | |
| | | | CAETHG_0382 | CLJU_c20350 | |
| 1716 | glycolate oxidase | 1.1.3.15 | CAETHG_0244 | CLJU_c21570 | CLRAG_31160 |
| 1717 | glycolate oxidase | 1.1.3.15 | CAETHG_3473 | CLJU_c13900 | CLRAG_09940 |
| 1718 | Succinyl-CoA synthetase, alpha subunit | | CAETHG_0431 | CLJU_c23670 | CLRAG_17500 |
| 1719 | hypothetical protein | | CAETHG_3907 | CLJU_c17980 | CLRAG_00650 |
| 1720 | Radical SAM superfamily enzyme YgiQ, UPF0313 | | CAETHG_0298 | CLJU_c22000 | CLRAG_31530 |
| | family | | | | |
| 1721 | Radical SAM superfamily enzyme, | | CAETHG_0786, | CLJU_c27030, | CLRAG_09540 |
| | MoaA/NifB/PqqE/SkfB family | | CAETHG_2300 | CLJU_c01970 | |
| 1722 | uncharacterized protein | | CAETHG_1274 | CLJU_c33760 | CLRAG_24520 |
| 1723 | hypothetical protein | | CAETHG_2018 | CLJU_c41890 | CLRAG_05000 |
| 1724 | radical SAM family uncharacterized protein | | CAETHG_2825 | CLJU_c07330 | CLRAG_26510 |
| 1725 | electron transport protein HydN | 1.12.1.4, | CAETHG_2799 | CLJU_c07080 | CLRAG_18930 |
| | | 1.1.99.33 | | | |
| 1726 | Fe2+ or Zn2+ uptake regulation protein | | CAETHG_2706 | CLJU_c06070 | CLRAG_07340 |
| 1727 | iron complex transport system ATP-binding | | CAETHG_2679 | CLJU_c05840 | CLRAG_07080 |
| | protein | | | | |
| 1728 | iron complex transport system substrate-binding | | CAETHG_2677 | CLJU_c05820 | CLRAG_07060 |
| | protein | | | | |
| 1729 | iron complex transport system permease protein | | CAETHG_2678 | CLJU_c05830 | CLRAG_07070 |
| 1730 | Uncharacterized 2Fe-2 and 4Fe—4S clusters- | | CAETHG_1606 | CLJU_c37530 | CLRAG_36940 |
| | containing protein, contains DUF4445 domain | | | | |
| 1731 | bacterioferritin | | CAETHG_0047 | CLJU_c19700 | CLRAG_39440 |
| 1732 | ferrous iron transport protein B | | CAETHG_0253 | CLJU_c21660 | CLRAG_31250 |
| 1733 | ferrous iron transport protein A | | CAETHG_0252 | CLJU_c21650 | CLRAG_31240 |
| 1734 | flagellar protein FliO/FliZ | | CAETHG_3123 | CLJU_c10330 | CLRAG_13040 |
| 1735 | flagellar operon protein | | CAETHG_3119 | CLJU_c10290 | CLRAG_13080 |
| 1736 | flagellar basal-body rod modification protein FlgD | | CAETHG_3118 | CLJU_c10280 | CLRAG_13090 |
| 1737 | flagellar hook protein FlgE | | CAETHG_3120 | CLJU_c10300 | CLRAG_13070 |
| 1738 | flagellar hook-length control protein FliK | | CAETHG_3117 | CLJU_c10270 | CLRAG_13100 |
| 1739 | chemotaxis protein MotB | | CAETHG_2252 | CLJU_c01460 | CLRAG_27120 |
| 1740 | flagellar motor switch protein FliN/FliY | | CAETHG_3043 | CLJU_c09480 | CLRAG_13620 |
| 1741 | c-di-GMP-binding flagellar brake protein YcgR, | | CAETHG_3130 | CLJU_c10400 | CLRAG_12970 |
| | contains PilZNR and PilZ domains | | | | |
| 1742 | flagellar protein FlbD | | CAETHG_3121 | CLJU_c10310 | CLRAG_13060 |
| 1743 | flagellar biosynthesis protein FlhG | | CAETHG_3129 | CLJU_c10390 | CLRAG_12980 |
| 1744 | NADH-FMN oxidoreductase RutF, flavin reductase | | CAETHG_0025 | CLJU_c19480 | CLRAG_39630 |
| | (DIM6/NTAB) family | | | | |
| 1745 | NADH-FMN oxidoreductase RutF, flavin reductase | | CAETHG_1451 | CLJU_c35430 | CLRAG_05970 |
| | (DIM6/NTAB) family | | | | |
| 1746 | Flavodoxin | | CAETHG_0034 | CLJU_c19570 | CLRAG_39560 |
| 1747 | Multimeric flavodoxin WrbA | | CAETHG_0543 | CLJU_c24780 | CLRAG_18010 |
| 1748 | Flavodoxin domain-containing protein | | CAETHG_1387 | CLJU_c34890 | CLRAG_26130 |
| 1749 | Flavodoxin | | CAETHG_3868 | CLJU_c17600 | CLRAG_01140 |
| 1750 | flavodoxin, short chain | | CAETHG_3483 | CLJU_c14000 | CLRAG_09330 |
| 1751 | hypothetical protein | | CAETHG_0220 | CLJU_c21340 | CLRAG_30900 |
| 1752 | Flavorubredoxin | | CAETHG_0291 | CLJU_c21940 | CLRAG_31490 |
| 1753 | hypothetical protein | | CAETHG_2057 | CLJU_c42330 | CLRAG_05440 |
| 1754 | FMN-dependent dehydrogenase, includes L-lactate | | CAETHG_1336 | CLJU_c34360 | CLRAG_14440 |
| | dehydrogenase and type II isopentenyl | | | | |
| | diphosphate isomerase | | | | |
| 1755 | peptidyl-prolyl cis-trans isomerase C | | CAETHG_2226 | CLJU_c01180 | CLRAG_30240 |
| 1756 | formate dehydrogenase major subunit | 1.2.1.43 | CAETHG_0084 | CLJU_c20040 | CLRAG_29330 |
| 1757 | FdhD protein | 1.1.99.33 | CAETHG_2793 | CLJU_c07020 | CLRAG_18870 |
| 1758 | Fur family transcriptional regulator, peroxide | | CAETHG_1463 | CLJU_c35550 | CLRAG_06080 |
| | stress response regulator | | | | |
| 1759 | type IV pilus assembly protein PilB | | CAETHG_3179 | CLJU_c10900 | CLRAG_12560 |
| 1760 | diguanylate cyclase (GGDEF) domain-containing | | CAETHG_0826 | CLJU_c28290 | CLRAG_34370 |
| | protein | | | | |
| 1761 | diguanylate cyclase (GGDEF) domain-containing | | CAETHG_1731 | CLJU_c38830 | CLRAG_21090 |
| | protein | | | | |
| 1762 | mannose-1-phosphate guanylyltransferase | | CAETHG_0735 | CLJU_c26540 | CLRAG_08420 |
| 1763 | putative glutamine amidotransferase | | CAETHG_1555 | CLJU_c36940 | CLRAG_36470 |
| 1764 | putative glutamine amidotransferase | | CAETHG_1911 | CLJU_c40680 | CLRAG_22870 |
| 1765 | uncharacterized protein | | CAETHG_0070 | CLJU_c19900 | CLRAG_39180 |
| 1766 | glycine cleavage system H protein | 1.8.1.4, | CAETHG_0475, | CLJU_c37540, | CLRAG_24840 |
| | | 2.1.2.10 | CAETHG_1607 | CLJU_c24170 | |
| 1767 | UDP:flavonoid glycosyltransferase YjiC, YdhE | | CAETHG_3490 | CLJU_c14090 | CLRAG_09440 |
| | family | | | | |
| 1768 | Predicted glycosyl hydrolase, GH43/DUF377 family | | CAETHG_2683 | CLJU_c05880 | CLRAG_07120 |
| 1769 | l,2-diacylglycerol-3-alpha-glucose alpha-1,2- | | CAETHG_0044 | CLJU_c19670 | CLRAG_39470 |
| | galactosyltransferase | | | | |
| 1770 | hypothetical protein | | CAETHG_0223, | CLJU_c12280, | CLRAG_30930 |
| | | | CAETHG_3310 | CLJU_c21370 | |
| 1771 | Glycosyltransferase involved in cell wall | | CAETHG_0736 | CLJU_c26550 | CLRAG_08430 |
| | bisynthesis | | | | |
| 1772 | UDP:flavonoid glycosyltransferase YjiC, YdhE | | CAETHG_0924 | CLJU_c29310 | CLRAG_35320 |
| | family | | | | |
| 1773 | Glycosyl transferase family 2 | | CAETHG_1251 | CLJU_c33520 | CLRAG_24760 |
| 1774 | Glycosyltransferase involved in cell wall | 2.4.1.52 | CAETHG_1303 | CLJU_c34050 | CLRAG_14200 |
| | bisynthesis | | | | |
| 1775 | Glycosyltransferase, catalytic subunit of cellulose | | CAETHG_1313 | CLJU_c34150 | CLRAG_14300 |
| | synthase and poly-beta-1,6-N-acetylglucosamine | | | | |
| | synthase | | | | |
| 1776 | Glycosyltransferase involved in cell wall | | CAETHG_1314 | CLJU_c34160 | CLRAG_14310 |
| | bisynthesis | | | | |
| 1777 | Glycosyltransferase involved in cell wall | | CAETHG_1315 | CLJU_c34170 | CLRAG_14320 |
| | bisynthesis | | | | |
| 1778 | dolichol-phosphate mannosyltransferase | | CAETHG_1737, | CLJU_c38890, | CLRAG_37810 |
| | | | CAETHG_2517 | CLJU_c04450 | |
| 1779 | Glycosyltransferase involved in cell wall | | CAETHG_2302 | CLJU_c01990 | CLRAG_27590 |
| | bisynthesis | | | | |
| 1780 | Glycosyltransferase involved in cell wall | | CAETHG_2309 | CLJU_c02040 | CLRAG_27640 |
| | bisynthesis | | | | |
| 1781 | Dolichyl-phosphate-mannose-protein | | CAETHG_2519 | CLJU_c04470 | CLRAG_37830 |
| | mannosyltransferase | | | | |
| 1782 | Glycosyltransferase involved in cell wall | | CAETHG_2527 | CLJU_c04550 | CLRAG_37910 |
| | bisynthesis | | | | |
| 1783 | exopolysaccharide biosynthesis polyprenyl | | CAETHG_2598, | CLJU_c05410, | CLRAG_38600 |
| | glycosylphosphotransferase | | CAETHG_2622 | CLJU_c05210 | |
| 1784 | rhamnosyltransferase | | CAETHG_2623 | CLJU_c05420 | CLRAG_38880 |
| 1785 | Glycosyl transferases group 1 | | CAETHG_2625 | CLJU_c05430 | CLRAG_38890 |
| 1786 | 4-amino-4-deoxy-L-arabinose transferase | | CAETHG_3709 | CLJU_c16140 | CLRAG_33110 |
| 1787 | Catechol 2,3-dioxygenase | | CAETHG_0065 | CLJU_c19850 | CLRAG_39230 |
| 1788 | DNA-binding transcriptional regulator, GntR family | | CAETHG_0068 | CLJU_c19880 | CLRAG_39200 |
| 1789 | putative GTP pyrophosphokinase | 2.7.6.5 | CAETHG_2376 | CLJU_c02740 | CLRAG_28340 |
| 1790 | GTP-binding protein HflX | | CAETHG_1289 | CLJU_c33910 | CLRAG_14060 |
| 1791 | GTP-binding protein | | CAETHG_1469 | CLJU_c35610 | CLRAG_06140 |
| 1792 | hypothetical protein | | CAETHG_3142 | CLJU_c10520 | CLRAG_12820 |
| 1793 | GTP-binding protein | | CAETHG_3329 | CLJU_c12470 | CLRAG_11330 |
| 1794 | ribosome biogenesis GTPase A | | CAETHG_3377 | CLJU_c12950 | CLRAG_10850 |
| 1795 | GTP-binding protein LepA | | CAETHG_2887 | CLJU_c07940 | CLRAG_25420 |
| 1796 | ribosome biogenesis GTPase | | CAETHG_3345 | CLJU_c12630 | CLRAG_11170 |
| 1797 | HDIG domain-containing protein | | CAETHG_1638 | CLJU_c38120 | CLRAG_37500 |
| 1798 | HD domain-containing protein | | CAETHG_2215 | CLJU_c01040 | CLRAG_30360 |
| 1799 | HD-GYP domain, c-di-GMP phosphodiesterase | | CAETHG_2708 | CLJU_c06090 | CLRAG_07370 |
| | class II (or its inactivated variant) | | | | |
| 1800 | 3′-5′ exoribonuclease | | CAETHG_1750 | CLJU_c39020 | CLRAG_21200 |
| 1801 | molecular chaperone GrpE | | CAETHG_2890 | CLJU_c07970 | CLRAG_25450 |
| 1802 | arsenite transporter, ACR3 family | | CAETHG_3666 | CLJU_c15670 | CLRAG_32730 |
| 1803 | copper ion binding protein | | CAETHG_0556 | CLJU_c24890 | CLRAG_17890 |
| 1804 | DNA helicase-2/ATP-dependent DNA helicase | | CAETHG_0292 | CLJU_c21950 | CLRAG_31500 |
| | PcrA | | | | |
| 1805 | DNA helicase-2/ATP-dependent DNA helicase | | CAETHG_0838 | CLJU_c28400 | CLRAG_34480 |
| | PcrA | | | | |
| 1806 | Superfamily II DNA or RNA helicase, SNF2 family | | CAETHG_3565 | CLJU_c14660 | CLRAG_20540 |
| 1807 | Predicted heme/steroid binding protein | | CAETHG_0859 | CLJU_c28640 | CLRAG_34720 |
| 1808 | histidine triad (HIT) family protein | 3.6.1.17 | CAETHG_2897 | CLJU_c08030 | CLRAG_08330 |
| 1809 | putative holliday junction resolvase | 4.1.2.4 | CAETHG_3299 | CLJU_c12170 | CLRAG_11630 |
| 1810 | hydroxylamine reductase | 1.7.99.1 | CAETHG_0812 | CLJU_c27260 | CLRAG_08960 |
| 1811 | Spore germination protein YaaH | | CAETHG_0066, | CLJU_c18760, | CLRAG_39220 |
| | | | CAETHG_3996 | CLJU_c19860 | |
| 1812 | Predicted amidohydrolase | | CAETHG_0225 | CLJU_c21390 | CLRAG_30950 |
| 1813 | hypothetical protein | | CAETHG_0559 | CLJU_c24920 | CLRAG_17860 |
| 1814 | L-ascorbate metabolism protein UlaG, beta- | | CAETHG_0696 | CLJU_c26200 | CLRAG_04280 |
| | lactamase superfamily | | | | |
| 1815 | ADP-ribose pyrophosphatase | 3.6.1.13 | CAETHG_1133 | CLJU_c32050 | CLRAG_02680 |
| 1816 | Metal-dependent hydrolase, beta-lactamase | | CAETHG_1283 | CLJU_c33850 | CLRAG_24430 |
| | superfamily II | | | | |
| 1817 | hypothetical protein | | CAETHG_1684 | CLJU_c38270 | CLRAG_20740 |
| 1818 | haloacid dehalogenase superfamily, subfamily IA, | | CAETHG_2015 | CLJU_c41860 | CLRAG_04970 |
| | variant 3 with third motif having DD or | | | | |
| | ED/haloacid dehalogenase superfamily, subfamily | | | | |
| | IA, variant 1 with third motif having Dx(3-4)D or | | | | |
| | Dx(3-4)E | | | | |
| 1819 | hypothetical protein | | CAETHG_2183 | CLJU_c00650 | CLRAG_19930 |
| 1820 | endoglucanase | | CAETHG_2220 | CLJU_c01090 | CLRAG_30310 |
| 1821 | endoglucanase | | CAETHG_2221 | CLJU_c01100 | CLRAG_30300 |
| 1822 | endoglucanase | | CAETHG_2222 | CLJU_c01110 | CLRAG_30290 |
| 1823 | L-ascorbate metabolism protein UlaG, beta- | | CAETHG_2290 | CLJU_c01870 | CLRAG_27470 |
| | lactamase superfamily | | | | |
| 1824 | Glyoxylase, beta-lactamase superfamily II | | CAETHG_2656 | CLJU_c05640 | CLRAG_06860 |
| 1825 | putative HD superfamily hydrolase of NAD | | CAETHG_2833 | CLJU_c07410 | CLRAG_26430 |
| | metabolism | | | | |
| 1826 | hydrolase | | CAETHG_2946 | CLJU_c08520 | CLRAG_07970 |
| 1827 | hypothetical protein | | CAETHG_3237 | CLJU_c11460 | CLRAG_11990 |
| 1828 | hypothetical protein | | CAETHG_3242 | CLJU_c11510 | CLRAG_11940 |
| 1829 | 8-oxo-dGTP pyrophosphatase MutT, NUDIX family | | CAETHG_3426 | CLJU_c13420 | CLRAG_10370 |
| 1830 | putative hydrolase of the HAD superfamily | | CAETHG_3815 | CLJU_c17030 | CLRAG_33910 |
| 1831 | hypothetical protein | | CAETHG_3889 | CLJU_c17810 | CLRAG_00900 |
| 1832 | Lysophospholipase L1 | | CAETHG_3944 | CLJU_c18370 | CLRAG_00300 |
| 1833 | hydroxylamine reductase | 1.7.99.1 | CAETHG_2866 | CLJU_c07730 | CLRAG_25270 |
| 1834 | Permease of the drug/metabolite transporter | | CAETHG_3966 | CLJU_c18590 | CLRAG_00110 |
| | (DMT) superfamily | | | | |
| 1835 | Phage integrase family protein | | CAETHG_0785 | CLJU_c27020 | CLRAG_09550 |
| 1836 | integrase/recombinase XerD | | CAETHG_3217 | CLJU_c11260 | CLRAG_12190 |
| 1837 | DJ-1/Pfpl family protein | | CAETHG_3867 | CLJU_c17590 | CLRAG_01150 |
| 1838 | voltage-gated potassium channel | | CAETHG_2684 | CLJU_c05890 | CLRAG_07130 |
| 1839 | Iron only hydrogenase large subunit, C-terminal | | CAETHG_0110 | CLJU_c20290 | CLRAG_26000 |
| | domain | | | | |
| 1840 | Alcohol dehydrogenase, class IV | 1.1.1.1, | CAETHG_2445 | CLJU_c03830 | CLRAG_28900 |
| | | 1.1.1.72, | | | |
| | | 1.1.1.21, | | | |
| | | 1.1.1.2 | | | |
| 1841 | 4Fe—4S dicluster domain-containing protein | | CAETHG_4056 | CLJU_c19210 | CLRAG_39860 |
| 1842 | Multimeric flavodoxin WrbA | | CAETHG_1082 | CLJU_c30780 | CLRAG_16220 |
| 1843 | NADPH-dependent FMN reductase | | CAETHG_3699 | CLJU_c16050 | CLRAG_33020 |
| 1844 | 2-dehydro-3-deoxyphosphogluconate aldolase/ | 4.1.2.14, | CAETHG_3443 | CLJU_c13610 | CLRAG_10260 |
| | (4S)-4-hydroxy-2-oxoglutarate aldolase | 4.1.3.16, | | | |
| | | 4.1.1.3 | | | |
| 1845 | lipid kinase, YegS/Rv2252/BmrU family | | CAETHG_2409 | CLJU_c02940 | CLRAG_28540 |
| 1846 | hypothetical protein | | CAETHG_3350 | CLJU_c12690 | CLRAG_11120 |
| 1847 | lactate permease | | CAETHG_0248 | CLJU_c21610 | CLRAG_31200 |
| 1848 | muramoyltetrapeptide carboxypeptidase | | CAETHG_2241 | CLJU_c01350 | CLRAG_27010 |
| 1849 | acetyl esterase | | CAETHG_1256 | CLJU_c33570 | CLRAG_24710 |
| 1850 | hypothetical protein | | CAETHG_3912 | CLJU_c18030 | CLRAG_00610 |
| 1851 | lipoate-protein ligase A | 2.7.7.63 | CAETHG_3015 | CLJU_c09210 | CLRAG_13820 |
| 1852 | lipoate-protein ligase A | 2.7.7.63 | CAETHG_1221 | CLJU_c33220 | CLRAG_15040 |
| 1853 | UDP-4-amino-4,6-dideoxy-N-acetyl-beta-L- | | CAETHG_2631 | CLJU_c05490 | CLRAG_38950 |
| | altrosamine transaminase | | | | |
| 1854 | L-lysine exporter family protein LysE/ArgO | | CAETHG_1838 | CLJU_c39920 | CLRAG_22170 |
| 1855 | Lysophospholipase L1 | | CAETHG_2947 | CLJU_c08530 | CLRAG_07960 |
| 1856 | regulatory helix-turn-helix protein, lysR family | | CAETHG_1047 | CLJU_c30420 | CLRAG_15930 |
| 1857 | DNA-binding transcriptional regulator, LysR family | | CAETHG_1210 | CLJU_c33120 | CLRAG_15200 |
| 1858 | DNA-binding transcriptional regulator, LysR family | | CAETHG_2037 | CLJU_c42070, | CLRAG_05180 |
| | | | | CLJU_c27740 | |
| 1859 | DNA-binding transcriptional regulator, LysR family | | CAETHG_2069, | CLJU_c42440, | CLRAG_01010 |
| | | | CAETHG_3880 | CLJU_c17720 | |
| 1860 | DNA-binding transcriptional regulator, LysR family | | CAETHG_2089 | CLJU_c42630 | CLRAG_05650 |
| 1861 | DNA-binding transcriptional regulator, LysR family | | CAETHG_3864 | CLJU_c17560 | CLRAG_01180 |
| 1862 | soluble lytic murein transglycosylase | | CAETHG_1257 | CLJU_c33590 | CLRAG_24690 |
| 1863 | septum formation protein | | CAETHG_2812 | CLJU_c07200 | CLRAG_26640 |
| 1864 | magnesium transporter | | CAETHG_0285 | CLJU_c21890 | CLRAG_31430 |
| 1865 | PRC-barrel domain-containing protein | | CAETHG_2412 | CLJU_c02970 | CLRAG_28580 |
| 1866 | o-succinylbenzoate synthase | | CAETHG_3735 | CLJU_c16410 | CLRAG_33210 |
| 1867 | manganese transport protein | | CAETHG_1334, | CLJU_c34340, | CLRAG_06350 |
| | | | CAETHG_1492 | CLJU_c35850 | |
| 1868 | DNA-binding transcriptional regulator, MarR family | | CAETHG_0393 | CLJU_c23290 | CLRAG_01440 |
| 1869 | DNA-binding transcriptional regulator, MarR family | | CAETHG_0620 | CLJU_c25510 | CLRAG_03720 |
| 1870 | DNA-binding transcriptional regulator, MarR family | | CAETHG_1748 | CLJU_c39000 | CLRAG_21180 |
| 1871 | MarR family protein | | CAETHG_2504 | CLJU_c04360 | CLRAG_37690 |
| 1872 | DNA-binding transcriptional regulator, MarR family | | CAETHG_3572, | CLJU_c17480, | CLRAG_20460 |
| | | | CAETHG_3861 | CLJU_c14730 | |
| 1873 | MarR family transcriptional regulator, 2-MHQ and | | CAETHG_3643 | CLJU_c15420 | CLRAG_24050 |
| | catechol-resistance regulon repressor | | | | |
| 1874 | small conductance mechanosensitive channel | | CAETHG_2107 | CLJU_c42810 | CLRAG_25620 |
| 1875 | GTP-binding protein | | CAETHG_3304 | CLJU_c12220 | CLRAG_11580 |
| 1876 | YidC/Oxal family membrane protein insertase | | CAETHG_2120 | CLJU_c42940 | CLRAG_25750 |
| 1877 | 4Fe—4S binding domain-containing protein | | CAETHG_3860 | CLJU_c17470 | CLRAG_01230 |
| 1878 | thiamine biosynthesis lipoprotein | | CAETHG_2065 | CLJU_c42400 | CLRAG_05540 |
| 1879 | regulator of sigma E protease | | CAETHG_3392 | CLJU_c13090 | CLRAG_10700 |
| 1880 | hypothetical protein | | CAETHG_2902 | CLJU_c08070 | CLRAG_08280 |
| 1881 | rhomboid protease GluP | | CAETHG_2709 | CLJU_c06100 | CLRAG_07380 |
| 1882 | Uncharacterized membrane protein YvbJ | | CAETHG_3093 | CLJU_c10020 | CLRAG_13340 |
| 1883 | HlyD family secretion protein | | CAETHG_3917 | CLJU_c18080 | CLRAG_00570 |
| 1884 | regulatory protein, luxR family | | CAETHG_3859 | CLJU_c17460 | CLRAG_01240 |
| 1885 | hypothetical protein | | CAETHG_0485 | CLJU_c24270 | CLRAG_24940 |
| 1886 | LexA-binding, inner membrane-associated putative | | CAETHG_0352 | CLJU_c22900 | CLRAG_01890 |
| | hydrolase | | | | |
| 1887 | Transglutaminase-like superfamily protein | | CAETHG_1447 | CLJU_c35390 | CLRAG_05930 |
| 1888 | diguanylate cyclase (GGDEF) domain-containing | | CAETHG_1514 | CLJU_c36050 | CLRAG_06540 |
| | protein | | | | |
| 1889 | zinc transport system ATP-binding protein | | CAETHG_1108 | CLJU_c31790 | CLRAG_02420 |
| 1890 | zinc transport system permease protein | | CAETHG_1109 | CLJU_c31800 | CLRAG_02430 |
| 1891 | Phosphoribosyl 1,2-cyclic phosphodiesterase | | CAETHG_2028 | CLJU_c41990 | CLRAG_05100 |
| 1892 | CTP:molybdopterin cytidylyltransferase MocA | | CAETHG_0465 | CLJU_c23990 | CLRAG_17170 |
| 1893 | HDIG domain-containing protein | | CAETHG_1673 | CLJU_c38170 | CLRAG_20630 |
| 1894 | HDIG domain-containing protein | 3.1.3.1 | CAETHG_2731 | CLJU_c06360 | CLRAG_30470 |
| 1895 | D-methionine transport system substrate-binding | | CAETHG_1138 | CLJU_c32100 | CLRAG_02730 |
| | protein | | | | |
| 1896 | D-methionine transport system permease protein | | CAETHG_2533 | CLJU_c04610 | CLRAG_37970 |
| 1897 | ABC-type Zn uptake system ZnuABC, Zn-binding component ZnuA | | CAETHG_1672 | CLJU_c38160 | CLRAG_20620 |
| 1898 | zinc transport system substrate-binding protein | | CAETHG_0318 | CLJU_c37880, | CLRAG_31780 |
| | | | | CLJU_c22200 | |
| 1899 | Uncharacterized 2Fe-2 and 4Fe—4S clusters- | | CAETHG_2846 | CLJU_c07530 | CLRAG_32340 |
| | containing protein, contains DUF4445 domain | | | | |
| 1900 | 7,8-dihydropterin-6-yl-methyl-4-(beta-D- | | CAETHG_1393 | CLJU_c34950 | CLRAG_26190 |
| | ribofuranosyl)aminobenzene 5′-phosphate | | | | |
| | synthase | | | | |
| 1901 | ribonuclease J | | CAETHG_3302 | CLJU_c12200 | CLRAG_11600 |
| 1902 | hypothetical protein | | CAETHG_0277 | CLJU_c21870 | CLRAG_31410 |
| 1903 | RNA processing exonuclease, beta-lactamase fold, | | CAETHG_1143 | CLJU_c32150 | CLRAG_02780 |
| | Cft2 family | | | | |
| 1904 | Rubrerythrin | | CAETHG_0302 | CLJU_c22040 | CLRAG_02720 |
| 1905 | methyl-accepting chemotaxis protein | | CAETHG_0353, | CLJU_c22910, | CLRAG_01880 |
| | | | CAETHG_1531 | CLJU_c36200 | |
| 1906 | Ligand-binding sensor domain-containing protein | | CAETHG_0920, | CLJU_c29270, | CLRAG_35280 |
| | | | CAETHG_2802 | CLJU_c07110 | |
| 1907 | methyl-accepting chemotaxis protein | | CAETHG_1062 | CLJU_c30560 | CLRAG_15980 |
| 1908 | methyl-accepting chemotaxis protein | | CAETHG_1248 | CLJU_c33490 | CLRAG_24790 |
| 1909 | methyl-accepting chemotaxis protein | | CAETHG_2454, | CLJU_c14810, | CLRAG_29030 |
| | | | CAETHG_3581 | CLJU_c03920 | |
| 1910 | methyl-accepting chemotaxis protein | | CAETHG_2457 | CLJU_c03950 | CLRAG_29060 |
| 1911 | methyl-accepting chemotaxis protein | | CAETHG_2549 | CLJU_c04770 | CLRAG_38150 |
| 1912 | methyl-accepting chemotaxis sensory transducer | | CAETHG_2701 | CLJU_c06030 | CLRAG_07260 |
| | with Cache sensor | | | | |
| 1913 | Methyl-accepting chemotaxis protein (MCP) | | CAETHG_0750 | CLJU_c26690 | CLRAG_08560 |
| | signalling domain-containing protein | | | | |
| 1914 | Methyl-accepting chemotaxis protein (MCP) | | CAETHG_2090 | CLJU_c42640 | CLRAG_05660 |
| | signalling domain-containing protein | | | | |
| 1915 | methyl-accepting chemotaxis protein | | CAETHG_2990 | CLJU_c08960 | CLRAG_07540 |
| 1916 | methyl-accepting chemotaxis protein | | CAETHG_0493 | CLJU_c24340 | CLRAG_25020 |
| 1917 | Methyl-accepting chemotaxis protein (MCP) | | CAETHG_0504, | CLJU_c37140, | CLRAG_30130 |
| | signalling domain-containing protein | | CAETHG_3957 | CLJU_c18500, | |
| | | | | CLJU_c24440 | |
| 1918 | Methyl-accepting chemotaxis protein (MCP) | | CAETHG_1569, | CLJU_c04190 | CLRAG_26870 |
| | signalling domain-containing protein | | CAETHG_2487 | | |
| 1919 | methyl-accepting chemotaxis protein | | CAETHG_3469 | CLJU_c13860 | CLRAG_09980 |
| 1920 | Methyltransferase domain-containing protein | | CAETHG_0515 | CLJU_c24540 | CLRAG_23640 |
| 1921 | Methyltransferase domain-containing protein | | CAETHG_0523 | CLJU_c24600 | CLRAG_18280 |
| 1922 | Ubiquinone/menaquinone biosynthesis C- | | CAETHG_0593 | CLJU_c25250 | CLRAG_03600 |
| | methylase UbiE | | | | |
| 1923 | tRNAl(Val) A37 N6-methylase TrmN6 | | CAETHG_2253 | CLJU_c01470 | CLRAG_27130 |
| 1924 | putative N6-adenine-specific DNA methylase | | CAETHG_2469 | CLJU_c04060 | CLRAG_29170 |
| 1925 | N-6 DNA Methylase | | CAETHG_2939 | CLJU_c08460 | CLRAG_08000 |
| 1926 | [methyl-Co(III) methanol-specific corrinoid | | CAETHG_0191, | CLJU_c21060, | CLRAG_30640 |
| | protein]:coenzyme M methyltransferase | | CAETHG_4046 | CLJU_c19120 | |
| 1927 | drug resistance transporter, EmrB/QacA subfamily | | CAETHG_0743 | CLJU_c26620 | CLRAG_08490 |
| 1928 | drug resistance transporter, EmrB/QacA subfamily | | CAETHG_0939 | CLJU_c29450 | CLRAG_35440 |
| 1929 | Transmembrane secretion effector | | CAETHG_1057, | CLJU_c02650, | CLRAG_15940 |
| | | | CAETHG_2366 | CLJU_c30520 | |
| 1930 | Major Facilitator Superfamily protein | | CAETHG_1080 | CLJU_c30760 | CLRAG_16200 |
| 1931 | Sugar phosphate permease | | CAETHG_3444 | CLJU_c13620 | CLRAG_10250 |
| 1932 | TatD DNase family protein | | CAETHG_2972 | CLJU_c08780 | CLRAG_07710 |
| 1933 | magnesium transporter | | CAETHG_2242 | CLJU_c01360 | CLRAG_27020 |
| 1934 | microcompartment protein PduB | | CAETHG_0341, | CLJU_c22790, | CLRAG_02000 |
| | | | CAETHG_3278 | CLJU_c11870 | |
| 1935 | BMC domain-containing protein | | CAETHG_1816, | CLJU_c39700, | CLRAG_21950 |
| | | | CAETHG_3290 | CLJU_c11990 | |
| 1936 | BMC domain-containing protein | | CAETHG_1817, | CLJU_c39710, | CLRAG_21960 |
| | | | CAETHG_3289 | CLJU_c11980 | |
| 1937 | ethanolamine utilization protein EutS | | CAETHG_3901 | CLJU_c17930 | CLRAG_00700 |
| 1938 | BMC domain-containing protein | | CAETHG_1820, | CLJU_c39740, | CLRAG_21990 |
| | | | CAETHG_3286 | CLJU_c11950 | |
| 1939 | Carboxysome shell and ethanolamine utilization | | CAETHG_1822 | CLJU_c39760 | CLRAG_22010 |
| | microcompartment protein CcmL/EutN | | | | |
| 1940 | BMC domain-containing protein | | CAETHG_1825, | CLJU_c39790, | CLRAG_22040 |
| | | | CAETHG_3283 | CLJU_c11920 | |
| 1941 | BMC domain-containing protein | | CAETHG_1831 | CLJU_c39850 | CLRAG_22100 |
| 1942 | BMC domain-containing protein | | CAETHG_1832 | CLJU_c39860 | CLRAG_22110 |
| 1943 | ethanolamine utilization protein EutN | | CAETHG_2801 | CLJU_c07100 | CLRAG_18950 |
| 1944 | release factor glutamine methyltransferase | | CAETHG_2330 | CLJU_c02250 | CLRAG_27850 |
| 1945 | molecular chaperone DnaK | | CAETHG_2891 | CLJU_c07980 | CLRAG_25460 |
| 1946 | molybdate transport system ATP-binding protein | | CAETHG_0313 | CLJU_c22150 | CLRAG_31730 |
| 1947 | molybdate transport system permease protein | | CAETHG_0314 | CLJU_c22160 | CLRAG_31740 |
| 1948 | molybdate transport system substrate-binding | | CAETHG_0315 | CLJU_c22170 | CLRAG_31750 |
| | protein | | | | |
| 1949 | putative molybdopterin biosynthesis protein | | CAETHG_2566 | CLJU_c04890 | CLRAG_38280 |
| 1950 | putative molybdopterin biosynthesis protein | | CAETHG_3823 | CLJU_c17110 | CLRAG_33980 |
| 1951 | cyclic pyranopterin monophosphate synthase | | CAETHG_1238 | CLJU_c33380 | CLRAG_14870 |
| | subunit MoaA | | | | |
| 1952 | cyclic pyranopterin phosphate synthase | | CAETHG_0573 | CLJU_c25050 | CLRAG_17730 |
| 1953 | xanthine dehydrogenase accessory factor | | CAETHG_0466 | CLJU_c24070, | CLRAG_17160 |
| | | | | CLJU_c24000 | |
| 1954 | molybdenum-pterin binding domain-containing | | CAETHG_0312 | CLJU_c22140 | CLRAG_31720 |
| | protein | | | | |
| 1955 | molybdenum cofactor synthesis domain- | | CAETHG_3904 | CLJU_c17950 | CLRAG_00680 |
| | containing protein | | | | |
| 1956 | Anaerobic selenocysteine-containing | | CAETHG_3653 | CLJU_c15540 | CLRAG_32620 |
| | dehydrogenase | | | | |
| 1957 | ModE molybdate transport repressor domain- | | CAETHG_0458 | CLJU_c23920 | CLRAG_17240 |
| | containing protein | | | | |
| 1958 | tRNA A37 threonylcarbamoyladenosine | | CAETHG_0226 | CLJU_c21400 | CLRAG_30960 |
| | dehydratase | | | | |
| 1959 | ATP-binding cassette, subfamily B | | CAETHG_2986 | CLJU_c08910 | CLRAG_07570 |
| 1960 | ATP-binding cassette, subfamily B | | CAETHG_2987 | CLJU_c08920 | CLRAG_07560 |
| 1961 | Membrane carboxypeptidase (penicillin-binding | | CAETHG_3692 | CLJU_c15910 | CLRAG_32960 |
| | protein) | | | | |
| 1962 | N-acetylmuramoyl-L-alanine amidase | | CAETHG_2291 | CLJU_c01880 | CLRAG_27480 |
| 1963 | sodium transport system ATP-binding protein | | CAETHG_3610 | CLJU_c15080 | CLRAG_24290 |
| 1964 | sodium transport system permease protein | | CAETHG_3609 | CLJU_c15070 | CLRAG_24300 |
| 1965 | putative efflux protein, MATE family | | CAETHG_1861 | CLJU_c40120 | CLRAG_22350 |
| 1966 | putative efflux protein, MATE family | | CAETHG_2938 | CLJU_c08450 | CLRAG_08010 |
| 1967 | putative efflux protein, MATE family | | CAETHG_3500, | CLJU_c14190, | CLRAG_09520 |
| | | | CAETHG_3720 | CLJU_c16260 | |
| 1968 | putative efflux protein, MATE family | | CAETHG_3625 | CLJU_c15230 | CLRAG_24120 |
| 1969 | Na+/H+-dicarboxylate symporter | | CAETHG_2499 | CLJU_c04310 | CLRAG_26750 |
| 1970 | solute:Na+ symporter, SSS family | | CAETHG_0135 | CLJU_c20530 | CLRAG_19470 |
| 1971 | hypothetical protein | | CAETHG_0410 | CLJU_c23460 | CLRAG_17650 |
| 1972 | Nitroreductase | | CAETHG_0080 | CLJU_c20000 | CLRAG_39010 |
| 1973 | Nitroreductase | | CAETHG_3639 | CLJU_c15370 | CLRAG_24090 |
| 1974 | 2,4-dienoyl-CoA reductase | | CAETHG_0482 | CLJU_c24240 | CLRAG_24910 |
| 1975 | 2,4-dienoyl-CoA reductase | | CAETHG_0205 | CLJU_c21190 | CLRAG_30770 |
| 1976 | 2,4-dienoyl-CoA reductase | | CAETHG_3603 | CLJU_c14990 | CLRAG_24360 |
| 1977 | hypothetical protein | , 1.1.1.2, | CAETHG_0555, | CLJU_c39950, | CLRAG_17900 |
| | | 1.1.1.1, | CAETHG_3954, | CLJU_c18470, | |
| | | 1.1.1.72, | CAETHG_1841 | CLJU_c24880 | |
| | | 1.1.1.21, | | | |
| | | 1.1.1.4 | | | |
| 1978 | NADPH-dependent FMN reductase | | CAETHG_0716 | CLJU_c26350 | CLRAG_04440 |
| 1979 | dinuclear metal center protein, YbgI/SA1388 | | CAETHG_2920 | CLJU_c08250 | CLRAG_08140 |
| | family | | | | |
| 1980 | hydrogenase maturation protease | | CAETHG_0860 | CLJU_c28650 | CLRAG_34730 |
| 1981 | ABC-type nitrate/sulfonate/bicarbonate transport | | CAETHG_2975 | CLJU_c08800 | CLRAG_07690 |
| | system, substrate-binding protein | | | | |
| 1982 | Nitroreductase | | CAETHG_1409 | CLJU_c35000 | CLRAG_26270 |
| 1983 | Nitroreductase | | CAETHG_1432 | CLJU_c35240 | CLRAG_05750 |
| 1984 | Nitroreductase | | CAETHG_1532 | CLJU_c36210 | CLRAG_23980 |
| 1985 | Nitroreductase | | CAETHG_3909 | CLJU_c18000 | CLRAG_00630 |
| 1986 | exonuclease SbcC | | CAETHG_0113 | CLJU_c20320 | CLRAG_25970 |
| 1987 | hypothetical protein | | CAETHG_0426 | CLJU_c23620 | CLRAG_17550 |
| 1988 | hypothetical protein | | CAETHG_2780 | CLJU_c06900 | CLRAG_18730 |
| 1989 | putative hydroxymethylpyrimidine transporter | | CAETHG_4057 | CLJU_c19220 | CLRAG_39850 |
| | CytX | | | | |
| 1990 | Nucleoside-diphosphate-sugar epimerase | 5.1.3.7, | CAETHG_3091 | CLJU_c10000 | CLRAG_13350 |
| | | 5.1.3.2 | | | |
| 1991 | Chromosome partitioning ATPase, Mrp family, | | CAETHG_1625, | CLJU_c01810, | CLRAG_37200 |
| | contains Fe—S cluster | | CAETHG_2284 | CLJU_c37780 | |
| 1992 | phosphatidate cytidylyltransferase | 2.7.7.41 | CAETHG_3390 | CLJU_c13070 | CLRAG_10720 |
| 1993 | ADP-ribose pyrophosphatase YjhB, NUDIX family | | CAETHG_0525 | CLJU_c24610 | CLRAG_18320 |
| 1994 | dATP pyrophosphohydrolase | | CAETHG_0527 | CLJU_c24630 | CLRAG_29990 |
| 1995 | ADP-ribose pyrophosphatase YjhB, NUDIX family | | CAETHG_3606 | CLJU_c15020 | CLRAG_24330 |
| 1996 | NUDIX domain-containing protein | | CAETHG_3872 | CLJU_c17640 | CLRAG_01100 |
| 1997 | Predicted O-methyltransferase YrrM | | CAETHG_3305 | CLJU_c12230 | CLRAG_11570 |
| 1998 | GTP-binding protein | | CAETHG_2831 | CLJU_c07390 | CLRAG_26450 |
| 1999 | knotted carbamoyltransferase YgeW | | CAETHG_0449 | CLJU_c23840 | CLRAG_17330 |
| 2000 | Uncharacterized OsmC-related protein | | CAETHG_0003 | CLJU_c19270 | CLRAG_39790 |
| 2001 | two-component system, OmpR family, sensor | | CAETHG_1797 | CLJU_c39520 | CLRAG_21720 |
| | histidine kinase KdpD | | | | |
| 2002 | Predicted oxidoreductase of the aldo/keto | | CAETHG_1059 | CLJU_c30540 | CLRAG_15970 |
| | reductase family | | | | |
| 2003 | Nitroreductase | | CAETHG_1146 | CLJU_c32180 | CLRAG_02810 |
| 2004 | Glycine/D-amino acid oxidase (deaminating) | | CAETHG_1255 | CLJU_c33560 | CLRAG_24720 |
| 2005 | sarcosine oxidase subunit alpha | 1.5.3.1 | CAETHG_1599 | CLJU_c37470 | CLRAG_36880 |
| 2006 | threonylcarbamoyladenosine tRNA | | CAETHG_2896 | CLJU_c08020 | CLRAG_08340 |
| | methylthiotransferase MtaB | | | | |
| 2007 | NADPH-dependent glutamate synthase beta chain | | CAETHG_2963 | CLJU_c08690 | CLRAG_07800 |
| 2008 | NAD(P)H-flavin reductase | | CAETHG_3965 | CLJU_c18580 | CLRAG_00120 |
| 2009 | putative selenate reductase | | CAETHG_0448 | CLJU_c23830 | CLRAG_17340 |
| 2010 | carbon-monoxide dehydrogenase small subunit | 1.1.1.204, | CAETHG_0455, | CLJU_c29930, | CLRAG_17270 |
| | | 1.17.1.4 | CAETHG_0992 | CLJU_c23890 | |
| 2011 | DNA-binding transcriptional regulator, PadR family | | CAETHG_1380 | CLJU_c34820 | CLRAG_26060 |
| 2012 | penicillin-binding protein 2 | | CAETHG_0109, | CLJU_c07250, | CLRAG_26010 |
| | | | CAETHG_2817 | CLJU_c20280 | |
| 2013 | BlaI family transcriptional regulator, penicillinase | | CAETHG_1721, | CLJU_c38720, | CLRAG_18580 |
| | repressor | | CAETHG_2769 | CLJU_c06780 | |
| 2014 | L-aminopeptidase/D-esterase | | CAETHG_0849 | CLJU_c28550 | CLRAG_34620 |
| 2015 | Zn-dependent protease (includes SpoIVFB) | | CAETHG_0966 | CLJU_c29680 | CLRAG_35680 |
| 2016 | hypothetical protein | | CAETHG_1592 | CLJU_c37360 | CLRAG_36800 |
| 2017 | Cell fate regulator YaaT, PSP1 superfamily | | CAETHG_2248 | CLJU_c01420 | CLRAG_27080 |
| | (controls sporulation, competence, biofilm | | | | |
| | development) | | | | |
| 2018 | spore coat assemly protein | | CAETHG_2311 | CLJU_c02060 | CLRAG_27660 |
| 2019 | Murein DD-endopeptidase MepM and murein | | CAETHG_2354 | CLJU_c02480 | CLRAG_28090 |
| | hydrolase activator NlpD, contain LysM domain | | | | |
| 2020 | hypothetical protein | | CAETHG_2565 | CLJU_c04880 | CLRAG_38270 |
| 2021 | Peptidase family S41 | | CAETHG_3718 | CLJU_c16240 | CLRAG_33150 |
| 2022 | Peptidase family S51 | | CAETHG_1074 | CLJU_c30700 | CLRAG_16130 |
| 2023 | peptide/nickel transport system substrate-binding protein | | CAETHG_1550 | CLJU_c36890 | CLRAG_36420 |
| 2024 | Cell division protein FtsI/penicillin-binding protein | | CAETHG_3307 | CLJU_c12250 | CLRAG_11550 |
| | 2 | | | | |
| 2025 | peptidoglycan glycosyltransferase | | CAETHG_2430 | CLJU_c03150 | CLRAG_28760 |
| 2026 | hypothetical protein | | CAETHG_0014 | CLJU_c19370 | CLRAG_39720 |
| 2027 | hypothetical protein | | CAETHG_0121 | CLJU_c20380 | CLRAG_25910 |
| 2028 | ABC-2 type transport system permease protein | | CAETHG_0355 | CLJU_c22930 | CLRAG_01860 |
| 2029 | ABC-2 type transport system permease protein | | CAETHG_0356 | CLJU_c22940 | CLRAG_01850 |
| 2030 | MFS transporter, NNP family, nitrate/nitrite | | CAETHG_0438 | CLJU_c23740 | CLRAG_17430 |
| | transporter | | | | |
| 2031 | putative MFS transporter, AGZA family, | | CAETHG_0461, | CLJU_c08480, | CLRAG_17210 |
| | xanthine/uracil permease | | CAETHG_2941 | CLJU_c23950 | |
| 2032 | Predicted PurR-regulated permease PerM | | CAETHG_0571 | CLJU_c25030 | CLRAG_17750 |
| 2033 | putative ABC transport system permease protein | | CAETHG_0647 | CLJU_c25780 | CLRAG_04010 |
| 2034 | hypothetical protein | | CAETHG_0729 | CLJU_c26480 | CLRAG_04570 |
| 2035 | putative ABC transport system permease protein | | CAETHG_0805 | CLJU_c27200 | CLRAG_20030 |
| 2036 | putative ABC transport system permease protein | | CAETHG_1015 | CLJU_c30150 | CLRAG_15730 |
| 2037 | Permease of the drug/metabolite transporter | | CAETHG_1141 | CLJU_c32130 | CLRAG_02760 |
| | (DMT) superfamily | | | | |
| 2038 | Predicted arabinose efflux permease, MFS family | | CAETHG_1151, | CLJU_c32230, | CLRAG_03050 |
| | | | CAETHG_2911 | CLJU_c08160 | |
| 2039 | Fucose permease | | CAETHG_1181 | CLJU_c32830 | CLRAG_15500 |
| 2040 | hypothetical protein | | CAETHG_1230 | CLJU_c33310 | CLRAG_14950 |
| 2041 | ABC-2 family transporter protein | | CAETHG_1583 | CLJU_c37270 | CLRAG_36710 |
| 2042 | putative ABC transport system permease protein | | CAETHG_1712 | CLJU_c38620 | CLRAG_20940 |
| 2043 | MFS transporter, DHA2 family, lincomycin | | CAETHG_2540 | CLJU_c04680 | CLRAG_38060 |
| | resistance protein | | | | |
| 2044 | Predicted arabinose efflux permease, MFS family | | CAETHG_2545 | CLJU_c04730 | CLRAG_38110 |
| 2045 | Permease of the drug/metabolite transporter | | CAETHG_2748 | CLJU_c06580 | CLRAG_18390 |
| | (DMT) superfamily | | | | |
| 2046 | ABC-2 type transport system permease protein | | CAETHG_3009 | CLJU_c09150 | CLRAG_13880 |
| 2047 | ABC-2 type transport system permease protein | | CAETHG_3010 | CLJU_c09160 | CLRAG_13870 |
| 2048 | hypothetical protein | | CAETHG_3271 | CLJU_c11800 | CLRAG_11740 |
| 2049 | Predicted PurR-regulated permease PerM | | CAETHG_3296 | CLJU_c12140 | CLRAG_11660 |
| 2050 | hypothetical protein | | CAETHG_3463 | CLJU_c13800 | CLRAG_10040 |
| 2051 | putative ABC transport system permease protein | | CAETHG_3511 | CLJU_c14290 | CLRAG_09650 |
| 2052 | putative membrane protein | | CAETHG_1135 | CLJU_c32070 | CLRAG_02700 |
| 2053 | putative membrane protein | | CAETHG_1136 | CLJU_c32080 | CLRAG_02710 |
| 2054 | YhgE/Pip N-terminal domain-containing | | CAETHG_1232 | CLJU_c33330 | CLRAG_14930 |
| | protein/YhgE/Pip C-terminal domain-containing | | | | |
| | protein | | | | |
| 2055 | DNA polymerase | | CAETHG_2274 | CLJU_c01720 | CLRAG_27320 |
| 2056 | chorismate mutase | | CAETHG_2806 | CLJU_c07140 | CLRAG_26700 |
| 2057 | Inositol hexakisphosphate | | CAETHG_1137, | CLJU_c38430, | CLRAG_08920 |
| | | | CAETHG_1698 | CLJU_c32090 | |
| 2058 | undecaprenyl-diphosphatase | | CAETHG_1152, | CLJU_c32240, | CLRAG_03120 |
| | | | CAETHG_3691 | CLJU_c15820 | |
| 2059 | PAP2 superfamily protein | | CAETHG_1683 | CLJU_c38260 | CLRAG_20730 |
| 2060 | phosphoglycolate phosphatase | 3.2.2.16, | CAETHG_2493 | CLJU_c04250 | CLRAG_26780 |
| | | 3.2.2.9 | | | |
| 2061 | undecaprenyl-diphosphatase | | CAETHG_3695 | CLJU_c15960 | CLRAG_22200 |
| 2062 | phosphoglycolate phosphatase | 3.2.2.16, | CAETHG_3886 | CLJU_c17780 | CLRAG_00930 |
| | | 3.2.2.9 | | | |
| 2063 | inorganic phosphate transporter, PiT family | | CAETHG_3327 | CLJU_c12450 | CLRAG_11350 |
| 2064 | two-component system, OmpR family, phosphate | | CAETHG_3320 | CLJU_c12380 | CLRAG_11420 |
| | regulon sensor histidine kinase PhoR | | | | |
| 2065 | PhoH-like ATPase | | CAETHG_0969 | CLJU_c29710 | CLRAG_35710 |
| 2066 | phosphate transport system protein | | CAETHG_3325 | CLJU_c12430 | CLRAG_11370 |
| 2067 | phosphocarrier protein | | CAETHG_3418 | CLJU_c13350 | CLRAG_10440 |
| 2068 | hypothetical protein | | CAETHG_1545 | CLJU_c36840 | CLRAG_36370 |
| 2069 | phosphoesterase, MJ0936 family | | CAETHG_2564 | CLJU_c04870 | CLRAG_38260 |
| 2070 | phosphoesterase RecJ domain-containing protein | | CAETHG_3400 | CLJU_c13170 | CLRAG_10620 |
| 2071 | putative hydrolase | | CAETHG_1250 | CLJU_c33510 | CLRAG_24770 |
| 2072 | Phosphoglycerate dehydrogenase | 1.1.1.95 | CAETHG_2039 | CLJU_c42090 | CLRAG_05200 |
| 2073 | probable phosphoglycerate mutase | 5.4.2.11 | CAETHG_0712 | CLJU_c26320 | CLRAG_04410 |
| 2074 | 3′,5′-cyclic AMP phosphodiesterase CpdA | | CAETHG_1521 | CLJU_c36110 | CLRAG_06640 |
| 2075 | hypothetical protein | | CAETHG_2374 | CLJU_c02720 | CLRAG_28320 |
| 2076 | Predicted phosphoribosyltransferase | | CAETHG_0721 | CLJU_c26400 | CLRAG_04490 |
| 2077 | dTDP-4-amino-4,6-dideoxygalactose transaminase | | CAETHG_0760 | CLJU_c26790 | CLRAG_08660 |
| 2078 | O-acetyl-ADP-ribose deacetylase (regulator of | | CAETHG_2691 | CLJU_c05940 | CLRAG_07170 |
| | RNase III), contains Macro domain | | | | |
| 2079 | UDP-N-acetylglucosamine 4,6-dehydratase | | CAETHG_2630 | CLJU_c05480 | CLRAG_38940 |
| | (inverting) | | | | |
| 2080 | pseudaminic acid synthase | 4.1.3.19, | CAETHG_2633 | CLJU_c05510 | CLRAG_38970 |
| | | 2.5.1.56 | | | |
| 2081 | UDP-2,4-diacetamido-2,4,6-trideoxy-beta-L- | 2.7.7.43 | CAETHG_2634 | CLJU_c05520 | CLRAG_38980 |
| | altropyranose hydrolase | | | | |
| 2082 | Membrane protein involved in the export of O- | | CAETHG_2635 | CLJU_c05530 | CLRAG_38990 |
| | antigen and teichoic acid | | | | |
| 2083 | spore coat polysaccharide biosynthesis protein | 2.7.7.38 | CAETHG_2632 | CLJU_c05500 | CLRAG_38960 |
| | SpsF | | | | |
| 2084 | Peptidoglycan/xylan/chitin deacetylase, | | CAETHG_0754 | CLJU_c26730 | CLRAG_08600 |
| | PgdA/CDA1 family | | | | |
| 2085 | Peptidoglycan/xylan/chitin deacetylase, | | CAETHG_0834, | CLJU_c38420, | CLRAG_34450 |
| | PgdA/CDAl family | | CAETHG_1697 | CLJU_c28370 | |
| 2086 | peptidoglycan-N-acetylmuramic acid deacetylase | | CAETHG_1297 | CLJU_c33990 | CLRAG_14140 |
| 2087 | Peptidoglycan/xylan/chitin deacetylase, | | CAETHG_1864, | CLJU_c40160, | CLRAG_07460 |
| | PgdA/CDAl family | | CAETHG_2718 | CLJU_c06180 | |
| 2088 | Peptidoglycan/xylan/chitin deacetylase, | | CAETHG_2265 | CLJU_c01600 | CLRAG_27250 |
| | PgdA/CDAl family | | | | |
| 2089 | Polysaccharide deacetylase | | CAETHG_2266 | CLJU_c01610 | CLRAG_27260 |
| 2090 | preprotein translocase subunit SecE | | CAETHG_1961 | CLJU_c41180 | CLRAG_23370 |
| 2091 | putative protease | | CAETHG_1339 | CLJU_c34390 | CLRAG_14470 |
| 2092 | tRNA threonylcarbamoyl adenosine modification | | CAETHG_1675 | CLJU_c38190 | CLRAG_20650 |
| | protein YeaZ | | | | |
| 2093 | carboxyl-terminal processing protease | | CAETHG_2424 | CLJU_c03090 | CLRAG_28700 |
| 2094 | carboxyl-terminal processing protease | | CAETHG_2703 | CLJU_c06040 | CLRAG_07310 |
| 2095 | hypothetical protein | | CAETHG_2704 | CLJU_c06050 | CLRAG_07320 |
| 2096 | 4-methyl-5(b-hydroxyethyl)-thiazole | | CAETHG_3016 | CLJU_c09220 | CLRAG_13810 |
| | monophosphate biosynthesis | | | | |
| 2097 | putative protease | | CAETHG_3306 | CLJU_c12240 | CLRAG_11560 |
| 2098 | ATP-dependent protease ClpP, protease subunit | | CAETHG_3407 | CLJU_c13240 | CLRAG_10550 |
| 2099 | hypothetical protein | | CAETHG_3600 | CLJU_c14960 | CLRAG_24390 |
| 2100 | serpin B | | CAETHG_3677 | CLJU_c15690 | CLRAG_32850 |
| 2101 | Peptidase family M28 | | CAETHG_2368 | CLJU_c02670 | CLRAG_28270 |
| 2102 | protein phosphatase | | CAETHG_3343 | CLJU_c12610 | CLRAG_11190 |
| 2103 | PAS domain S-box-containing protein | | CAETHG_2404 | CLJU_c02890 | CLRAG_28490 |
| 2104 | preprotein translocase subunit SecF | | CAETHG_1272 | CLJU_c33740 | CLRAG_24540 |
| 2105 | preprotein translocase subunit SecD | | CAETHG_1273 | CLJU_c33750 | CLRAG_24530 |
| 2106 | purine catabolism regulatory protein | | CAETHG_0433 | CLJU_c23690 | CLRAG_17480 |
| 2107 | Cytidine and deoxycytidylate deaminase zinc- | 3.5.4.1 | CAETHG_0990 | CLJU_c29910 | CLRAG_35910 |
| | binding region | | | | |
| 2108 | NAD+ diphosphatase | | CAETHG_0028 | CLJU_c19510 | CLRAG_39600 |
| 2109 | pyrimidine operon attenuation protein/uracil | 2.4.2.9 | CAETHG_3164 | CLJU_c10740 | CLRAG_12600 |
| | phosphoribosyltransferase | | | | |
| 2110 | pyruvate formate lyase activating enzyme | | CAETHG_3449 | CLJU_c13670 | CLRAG_10200 |
| 2111 | 4-hydroxy-tetrahydrodipicolinate synthase | 4.2.1.52 | CAETHG_2178 | CLJU_c00600 | CLRAG_19980 |
| 2112 | uncharacterized radical SAM protein YgiQ | | CAETHG_2411 | CLJU_c02960 | CLRAG_28570 |
| 2113 | GTP-binding protein Era | | CAETHG_2905 | CLJU_c08100 | CLRAG_08250 |
| 2114 | fumarate reductase flavoprotein subunit | 1.3.99.1 | CAETHG_2961 | CLJU_c08670 | CLRAG_07820 |
| 2115 | transcriptional regulator, AbrB family | | CAETHG_2257 | CLJU_c01510 | CLRAG_27170 |
| 2116 | DNA-binding transcriptional activator of the SARP | | CAETHG_0235 | CLJU_c21490 | CLRAG_31050 |
| | family | | | | |
| 2117 | PucR C-terminal helix-turn-helix domain- | | CAETHG_1081 | CLJU_c30770 | CLRAG_16210 |
| | containing protein | | | | |
| 2118 | regulatory protein | | CAETHG_1448 | CLJU_c35400 | CLRAG_05940 |
| 2119 | bla regulator protein blaR1 | | CAETHG_3432 | CLJU_c13480 | CLRAG_10310 |
| 2120 | bla regulator protein blaR1 | | CAETHG_4027 | CLJU_c18930 | CLRAG_40040 |
| 2121 | site-specific DNA recombinase | | CAETHG_1440, | CLJU_c35310, | CLRAG_05840 |
| | | | CAETHG_3959 | CLJU_c18520 | |
| 2122 | DNA-binding response regulator, OmpR family, | | CAETHG_0061 | CLJU_c19810 | CLRAG_39270 |
| | contains REC and winged-helix (wHTH) domain | | | | |
| 2123 | diguanylate cyclase (GGDEF) domain-containing | | CAETHG_1072 | CLJU_c30680 | CLRAG_16100 |
| | protein | | | | |
| 2124 | DNA-binding response regulator, OmpR family, | | CAETHG_2216 | CLJU_c01050 | CLRAG_30350 |
| | contains REC and winged-helix (wHTH) domain | | | | |
| 2125 | pilus assembly protein CpaE | | CAETHG_0878 | CLJU_c28820 | CLRAG_34900 |
| 2126 | membrane protein | | CAETHG_1777 | CLJU_c39320 | CLRAG_21530 |
| 2127 | ribonuclease G | | CAETHG_2827 | CLJU_c07350 | CLRAG_26490 |
| 2128 | ribonuclease HI | | CAETHG_2712 | CLJU_c06130 | CLRAG_07410 |
| 2129 | ribonuclease R | | CAETHG_1754 | CLJU_c39090 | CLRAG_21240 |
| 2130 | 23S rRNA pseudouridine955/2504/2580 synthase | 4.2.1.70 | CAETHG_3848 | CLJU_c17350 | CLRAG_29230 |
| 2131 | Ribosomal protein L7Ae | | CAETHG_3397 | CLJU_c13140 | CLRAG_10650 |
| 2132 | ribosome recycling factor | | CAETHG_3388 | CLJU_c13050 | CLRAG_10740 |
| 2133 | ATP-dependent RNA helicase DeaD | | CAETHG_1450 | CLJU_c35420 | CLRAG_05960 |
| 2134 | RNA polymerase sigma-70 factor, ECF subfamily | | CAETHG_0730 | CLJU_c26490 | CLRAG_08370 |
| 2135 | RNA polymerase sigma factor, sigma-70 family | | CAETHG_2597 | CLJU_c05200 | CLRAG_38590 |
| 2136 | RNA polymerase sigma-70 factor, ECF subfamily | | CAETHG_2772 | CLJU_c06810 | CLRAG_18610 |
| 2137 | RNA polymerase sigma-70 factor, ECF subfamily | | CAETHG_3440 | CLJU_c13570 | CLRAG_10290 |
| 2138 | RNA polymerase sigma factor, sigma-70 family | | CAETHG_0660 | CLJU_c25910 | CLRAG_04050 |
| 2139 | hypothetical protein | | CAETHG_1965 | CLJU_c41240 | CLRAG_23430 |
| 2140 | spoIIIJ-associated protein | | CAETHG_2119 | CLJU_c42930 | CLRAG_25740 |
| 2141 | RNA-binding protein YlmH, contains S4-like | | CAETHG_3158 | CLJU_c10680 | CLRAG_12660 |
| | domain | | | | |
| 2142 | Predicted component of the ribosome quality | | CAETHG_3165 | CLJU_c10750 | CLRAG_12590 |
| | control (RQC) complex, YloA/Tae2 family, contains | | | | |
| | fibronectin-binding (FbpA) and DUF814 domains | | | | |
| 2143 | S1 RNA binding domain protein | | CAETHG_1991 | CLJU_c41620 | CLRAG_04750 |
| 2144 | Na+-translocating ferredoxin:NAD+ | 1.16.1.4 | CAETHG_1823 | CLJU_c39770 | CLRAG_22020 |
| | oxidoreductase RNF, RnfC subunit | | | | |
| 2145 | rod shape-determining protein MreD | | CAETHG_2816 | CLJU_c07240 | CLRAG_26600 |
| 2146 | rod shape-determining protein MreC | | CAETHG_2815 | CLJU_c07230 | CLRAG_26610 |
| 2147 | rod shape determining protein RodA | | CAETHG_2821 | CLJU_c07290 | CLRAG_26550 |
| 2148 | Putative rRNA methylase | | CAETHG_0413 | CLJU_c23490 | CLRAG_17620 |
| 2149 | RNA methyltransferase, TrmH family | | CAETHG_1343 | CLJU_c34430 | CLRAG_14510 |
| 2150 | tRNA (cytidine/uridine-2′-O-)-methyltransferase | | CAETHG_1764 | CLJU_c39190 | CLRAG_21340 |
| 2151 | 23S rRNA (guanosine2251-2′-O)-methyltransferase | | CAETHG_1966 | CLJU_c41250 | CLRAG_23440 |
| 2152 | 23S rRNA (cytidinel920-2′-O)/16S rRNA | | CAETHG_3206 | CLJU_c11170 | CLRAG_12290 |
| | (cytidinel409-2′-O)-methyltransferase | | | | |
| 2153 | 16S rRNA (cytosine967-C5)-methyltransferase | | CAETHG_3341 | CLJU_c12590 | CLRAG_11210 |
| 2154 | Rubrerythrin | | CAETHG_0664 | CLJU_c25950 | CLRAG_04090 |
| 2155 | Rubrerythrin | | CAETHG_0887 | CLJU_c28910 | CLRAG_34990 |
| 2156 | Rubrerythrin | | CAETHG_1779 | CLJU_c39340 | CLRAG_21550 |
| 2157 | Rubrerythrin | | CAETHG_1791 | CLJU_c39460 | CLRAG_21670 |
| 2158 | Rubrerythrin | | CAETHG_3813 | CLJU_c17010 | CLRAG_33880 |
| 2159 | 16S rRNA (cytosinel402-N4)-methyltransferase | | CAETHG_3144 | CLJU_c10540 | CLRAG_12800 |
| 2160 | 2-polyprenyl-3-methyl-5-hyd roxy-6-metoxy-1,4- | | CAETHG_1355 | CLJU_c34590 | CLRAG_14670 |
| | benzoquinol methylase | | | | |
| 2161 | Ribosomal 50S subunit-recycling heat shock | | CAETHG_1995 | CLJU_c41660 | CLRAG_04790 |
| | protein, contains S4 domain | | | | |
| 2162 | Methyltransferase domain-containing protein | | CAETHG_0944 | CLJU_c29500 | CLRAG_35490 |
| 2163 | Trans-aconitate methyltransferase | | CAETHG_3173 | CLJU_c10840 | CLRAG_03140 |
| 2164 | RND family efflux transporter, MFP subunit | | CAETHG_0390 | CLJU_c23270 | CLRAG_01460 |
| 2165 | HlyD family secretion protein | | CAETHG_2506, | CLJU_c04380, | CLRAG_37710 |
| | | | CAETHG_2693 | CLJU_c05960 | |
| 2166 | RND family efflux transporter, MFP subunit | | CAETHG_2471, | CLJU_c07590, | CLRAG_29190 |
| | | | CAETHG_2852 | CLJU_c04080 | |
| 2167 | His Kinase A (phospho-acceptor) domain- | | CAETHG_2515 | CLJU_c04430 | CLRAG_37790 |
| | containing protein | | | | |
| 2168 | Signal transduction histidine kinase | | CAETHG_2786 | CLJU_c06960 | CLRAG_18800 |
| 2169 | PAS domain S-box-containing protein | | CAETHG_1004 | CLJU_c30050 | CLRAG_15680 |
| 2170 | Signal transduction histidine kinase | | CAETHG_2550 | CLJU_c04780 | CLRAG_38160 |
| 2171 | PAS domain S-box-containing protein | | CAETHG_0056 | CLJU_c19760 | CLRAG_39380 |
| 2172 | HAMP domain-containing protein | | CAETHG_0577 | CLJU_c25090 | CLRAG_17680 |
| 2173 | DNA-binding response regulator, OmpR family, | | CAETHG_0803 | CLJU_c27180 | CLRAG_20050 |
| | contains REC and winged-helix (wHTH) domain | | | | |
| 2174 | Signal transduction histidine kinase | | CAETHG_1013 | CLJU_c30130 | CLRAG_15710 |
| 2175 | His Kinase A (phospho-acceptor) domain- | | CAETHG_1068 | CLJU_c30640 | CLRAG_16070 |
| | containing protein | | | | |
| 2176 | Signal transduction histidine kinase | | CAETHG_1714 | CLJU_c38640 | CLRAG_20960 |
| 2177 | Signal transduction histidine kinase | | CAETHG_1872 | CLJU_c40260 | CLRAG_07650 |
| 2178 | HAMP domain-containing protein | | CAETHG_2004 | CLJU_c41750 | CLRAG_04880 |
| 2179 | Signal transduction histidine kinase | | CAETHG_2217 | CLJU_c01060 | CLRAG_30340 |
| 2180 | His Kinase A (phospho-acceptor) domain- | | CAETHG_3460 | CLJU_c13770 | CLRAG_10070 |
| | containing protein | | | | |
| 2181 | Signal transduction histidine kinase | | CAETHG_3602 | CLJU_c14980 | CLRAG_24370 |
| 2182 | DNA-binding response regulator, OmpR family, | | CAETHG_2005 | CLJU_c41760 | CLRAG_04890 |
| | contains REC and winged-helix (wHTH) domain | | | | |
| 2183 | diguanylate cyclase (GGDEF) domain-containing | | CAETHG_2642 | CLJU_c05600 | CLRAG_06730 |
| | protein | | | | |
| 2184 | cell division topological specificity factor | | CAETHG_2820 | CLJU_c07280 | CLRAG_26560 |
| 2185 | septum site-determining protein MinD | | CAETHG_2819 | CLJU_c07270 | CLRAG_26570 |
| 2186 | serine protease Do | | CAETHG_2003 | CLJU_c41740 | CLRAG_04870 |
| 2187 | serine protease Do | | CAETHG_2936 | CLJU_c08430 | CLRAG_08030 |
| 2188 | putative serine protein kinase, PrkA | | CAETHG_2503 | CLJU_c04350 | CLRAG_37680 |
| 2189 | Lysophospholipase L1 | | CAETHG_1718, | CLJU_c38700, | CLRAG_08050 |
| | | | CAETHG_2934 | CLJU_c08400 | |
| 2190 | NADP-dependent 3-hydroxy acid dehydrogenase | | CAETHG_3497 | CLJU_c14160 | CLRAG_09480 |
| | YdfG | | | | |
| 2191 | hypothetical protein | 2.3.1.85, | CAETHG_0180 | CLJU_c20960 | CLRAG_19060 |
| | | 2.3.1.86, | | | |
| | | 1.1.1.100, | | | |
| | | 1.1.1.0 | | | |
| 2192 | 3-oxoacyl-[acyl-carrier protein] reductase | 2.3.1.85, | CAETHG_0914 | CLJU_c29220 | CLRAG_35220 |
| | | 2.3.1.86, | | | |
| | | 1.1.1.100, | | | |
| | | 1.1.1.0 | | | |
| 2193 | hypothetical protein | 2.3.1.85, | CAETHG_0739 | CLJU_c26580 | CLRAG_08450 |
| | | 2.3.1.86, | | | |
| | | 1.1.1.100, | | | |
| | | 1.1.1.0 | | | |
| 2194 | RNA polymerase sigma-70 factor, ECF subfamily | | CAETHG_3678 | CLJU_c15700 | CLRAG_32860 |
| 2195 | RNA polymerase sigma-70 factor, ECF subfamily | | CAETHG_2488 | CLJU_c04200 | CLRAG_26860 |
| 2196 | Transcriptional regulator of acetoin/glycerol | | CAETHG_0386, | CLJU_c23230 | CLRAG_01500 |
| | metabolism | | CAETHG_2800 | | |
| 2197 | diguanylate cyclase (GGDEF) domain-containing | | CAETHG_3871 | CLJU_c17630 | CLRAG_01110 |
| | protein | | | | |
| 2198 | signal peptidase II | | CAETHG_3161 | CLJU_c10710 | CLRAG_12630 |
| 2199 | two-component system, chemotaxis family, | | CAETHG_1003 | CLJU_c30040 | CLRAG_15670 |
| | response regulator CheY | | | | |
| 2200 | two-component system, chemotaxis family, | | CAETHG_3429 | CLJU_c13450 | CLRAG_10340 |
| | response regulator CheY | | | | |
| 2201 | Signal transduction histidine kinase | | CAETHG_0023 | CLJU_c19460 | CLRAG_39650 |
| 2202 | Signal transduction histidine kinase | | CAETHG_0040 | CLJU_c19630 | CLRAG_39510 |
| 2203 | Signal transduction histidine kinase | | CAETHG_0062 | CLJU_c19820 | CLRAG_39260 |
| 2204 | hypothetical protein | | CAETHG_2528 | CLJU_c04560 | CLRAG_37920 |
| 2205 | EAL and modified HD-GYP domain-containing | | CAETHG_0011 | CLJU_c19340 | CLRAG_39750 |
| | signal transduction protein | | | | |
| 2206 | PAS domain S-box-containing protein/diguanylate | | CAETHG_1070 | CLJU_c30660 | CLRAG_16080 |
| | cyclase (GGDEF) domain-containing protein | | | | |
| 2207 | PAS domain S-box-containing protein/diguanylate | | CAETHG_3969 | CLJU_c18610 | CLRAG_00040 |
| | cyclase (GGDEF) domain-containing protein | | | | |
| 2208 | c-di-AMP phosphodiesterase, consists of a GGDEF- | | CAETHG_2099 | CLJU_c42740 | CLRAG_25550 |
| | like and DHH domains | | | | |
| 2209 | Single-stranded DNA-binding protein | | CAETHG_1358 | CLJU_c34620 | CLRAG_14700 |
| 2210 | phosphoesterase RecJ domain protein | | CAETHG_1271 | CLJU_c33730 | CLRAG_24550 |
| 2211 | selenium metabolism protein YedF | | CAETHG_0224 | CLJU_c21380 | CLRAG_30940 |
| 2212 | Site-specific recombinase XerD | | CAETHG_0186 | CLJU_c21010 | CLRAG_19010 |
| 2213 | Small, acid-soluble spore protein, alpha/beta type | | CAETHG_0276, | CLJU_c28580, | CLRAG_34650 |
| | | | CAETHG_0853 | CLJU_c21860 | |
| 2214 | Small, acid-soluble spore protein, alpha/beta type | | CAETHG_0541 | CLJU_c24760 | CLRAG_18060 |
| 2215 | Small, acid-soluble spore protein, alpha/beta type | | CAETHG_0971 | CLJU_c30960, | CLRAG_35720 |
| | | | | CLJU_c29720 | |
| 2216 | small acid-soluble spore protein H (minor) | | CAETHG_0980 | CLJU_c29810 | CLRAG_35820 |
| 2217 | Small, acid-soluble spore protein, alpha/beta type | | CAETHG_1354 | CLJU_c34580 | CLRAG_14660 |
| 2218 | DNA processing protein | | CAETHG_3382 | CLJU_c12990 | CLRAG_10800 |
| 2219 | small multidrug resistance pump | | CAETHG_3645 | CLJU_c15440 | CLRAG_32570 |
| 2220 | Superfamily II DNA or RNA helicase, SNF2 family | | CAETHG_0242 | CLJU_c21550 | CLRAG_31110 |
| 2221 | proton glutamate symport protein | | CAETHG_0164 | CLJU_c20790 | CLRAG_19210 |
| 2222 | solute:Na+ symporter, SSS family | | CAETHG_1185 | CLJU_c32870 | CLRAG_15460 |
| 2223 | solute:Na+ symporter, SSS family | | CAETHG_3470 | CLJU_c13870 | CLRAG_09970 |
| 2224 | divalent anion:Na+ symporter, DASS family | | CAETHG_0819 | CLJU_c28190 | CLRAG_09140 |
| 2225 | chromosome partitioning protein, ParB family | | CAETHG_2113 | CLJU_c42870 | CLRAG_25680 |
| 2226 | spore coat protein JB | | CAETHG_1241 | CLJU_c33410 | CLRAG_14840 |
| 2227 | dTDP-4-amino-4,6-dideoxygalactose transaminase | | CAETHG_0759 | CLJU_c26780 | CLRAG_08650 |
| 2228 | Coat F domain-containing protein | | CAETHG_1253 | CLJU_c33540 | CLRAG_24740 |
| 2229 | spore coat protein, CotS family | | CAETHG_2304 | CLJU_c02000 | CLRAG_27600 |
| 2230 | hypothetical protein | | CAETHG_2306 | CLJU_c02020 | CLRAG_27620 |
| 2231 | spore coat protein, CotS family | | CAETHG_2308 | CLJU_c02030 | CLRAG_27630 |
| 2232 | spore coat-associated protein S | | CAETHG_2310 | CLJU_c02050 | CLRAG_27650 |
| 2233 | spore coat protein, CotS family | | CAETHG_2915 | CLJU_c08200 | CLRAG_08190 |
| 2234 | spore coat protein JC | | CAETHG_1242 | CLJU_c33420 | CLRAG_14830 |
| 2235 | Spore coat protein CotF | | CAETHG_0978 | CLJU_c29790 | CLRAG_35790 |
| 2236 | Coat F domain-containing protein | | CAETHG_2174 | CLJU_c00560 | CLRAG_20010 |
| 2237 | spore germination protein KA | | CAETHG_1503 | CLJU_c35950 | CLRAG_06450 |
| 2238 | germination protein, Ger(x)C family | | CAETHG_1504 | CLJU_c35960 | CLRAG_06460 |
| 2239 | spore germination protein (amino acid permease) | | CAETHG_1506 | CLJU_c35980 | CLRAG_06480 |
| 2240 | spore germination protein KB | | CAETHG_3738 | CLJU_c16440, | CLRAG_33240 |
| | | | | CLJU_c16460 | |
| 2241 | spore germination protein KC | | CAETHG_3743 | CLJU_c16480 | CLRAG_33260 |
| 2242 | spore germination protein KA | | CAETHG_3744 | CLJU_c16490 | CLRAG_33270 |
| 2243 | germination protein, Ger(x)C family | | CAETHG_3951 | CLJU_c18440 | CLRAG_00240 |
| 2244 | spore germination protein (amino acid permease) | | CAETHG_3952 | CLJU_c18450 | CLRAG_00230 |
| 2245 | GerA spore germination protein | | CAETHG_3953 | CLJU_c18460 | CLRAG_00220 |
| 2246 | spore germination protein | | CAETHG_2318 | CLJU_c02130 | CLRAG_27730 |
| 2247 | spore germination protein (amino acid | | CAETHG_2319 | CLJU_c02140 | CLRAG_27740 |
| | permease)/germination protein, Ger(x)C family | | | | |
| 2248 | spore maturation protein A | | CAETHG_2272 | CLJU_c01700 | CLRAG_27300 |
| 2249 | spore maturation protein B | | CAETHG_2273 | CLJU_c01710 | CLRAG_27310 |
| 2250 | spore photoproduct lyase | 4.1.99.— | CAETHG_1200 | CLJU_c33020 | CLRAG_15310 |
| 2251 | stage II sporulation protein GA (sporulation sigma- | | CAETHG_3313 | CLJU_c12310 | CLRAG_11490 |
| | E factor processing peptidase) | | | | |
| 2252 | sporulation integral membrane protein YlbJ | | CAETHG_3355 | CLJU_c12740 | CLRAG_11070 |
| 2253 | germination protein YpeB | | CAETHG_2322 | CLJU_c02170 | CLRAG_27770 |
| 2254 | sporulation protein, YlmC/YmxH family | | CAETHG_3316 | CLJU_c12340 | CLRAG_11460 |
| 2255 | SsrA-binding protein | | CAETHG_1753 | CLJU_c39080 | CLRAG_21230 |
| 2256 | stage II sporulation protein D | | CAETHG_2353 | CLJU_c02470 | CLRAG_28080 |
| 2257 | stage II sporulation protein E | | CAETHG_1990 | CLJU_c41560 | CLRAG_04690 |
| 2258 | stage II sporulation protein M | | CAETHG_3215 | CLJU_c11250 | CLRAG_12210 |
| 2259 | stage II sporulation protein P | | CAETHG_2885 | CLJU_c07920 | CLRAG_25400 |
| 2260 | stage II sporulation protein R | | CAETHG_2320 | CLJU_c02150 | CLRAG_27750 |
| 2261 | stage III sporulation protein AA | | CAETHG_3192 | CLJU_c11030 | CLRAG_12430 |
| 2262 | stage III sporulation protein AB | | CAETHG_3193 | CLJU_c11040 | CLRAG_12420 |
| 2263 | stage III sporulation protein AC | | CAETHG_3194 | CLJU_c11050 | CLRAG_12410 |
| 2264 | stage III sporulation protein AD | | CAETHG_3195 | CLJU_c11060 | CLRAG_12400 |
| 2265 | stage III sporulation protein AE | | CAETHG_3196 | CLJU_c11070 | CLRAG_12390 |
| 2266 | stage III sporulation protein AF | | CAETHG_3197 | CLJU_c11080 | CLRAG_12380 |
| 2267 | stage III sporulation protein AG | | CAETHG_3198 | CLJU_c11090 | CLRAG_12370 |
| 2268 | stage III sporulation protein AH | | CAETHG_3199 | CLJU_c11100 | CLRAG_12360 |
| 2269 | stage IV sporulation protein B | | CAETHG_3211 | CLJU_c11210 | CLRAG_12250 |
| 2270 | stage IV sporulation protein FB | | CAETHG_2824 | CLJU_c07320 | CLRAG_26520 |
| 2271 | stage V sporulation protein AC | | CAETHG_0176 | CLJU_c20910 | CLRAG_19100 |
| 2272 | stage V sporulation protein AE | | CAETHG_0178 | CLJU_c20940 | CLRAG_19080 |
| 2273 | stage V sporulation protein B | | CAETHG_0303 | CLJU_c22050 | CLRAG_31570 |
| 2274 | stage V sporulation protein B | | CAETHG_1998 | CLJU_c41690 | CLRAG_04820 |
| 2275 | stage V sporulation protein B | | CAETHG_3849 | CLJU_c17360 | CLRAG_29220 |
| 2276 | stage V sporulation protein G | | CAETHG_2008 | CLJU_c41790 | CLRAG_04920 |
| 2277 | stage V sporulation protein R | | CAETHG_2501 | CLJU_c04330 | CLRAG_37660 |
| 2278 | MFS transporter, SP family, major inositol | | CAETHG_3686 | CLJU_c15780 | CLRAG_32940 |
| | transporter | | | | |
| 2279 | Subtilase family protein | | CAETHG_2038 | CLJU_c42080 | CLRAG_05190 |
| 2280 | methyl-galactoside transport system substrate- | | CAETHG_2989 | CLJU_c08950 | CLRAG_07550 |
| | binding protein | | | | |
| 2281 | carbohydrate diacid regulator | | CAETHG_0822 | CLJU_c28220 | CLRAG_09170 |
| 2282 | sugar fermentation stimulation protein A | | CAETHG_2204 | CLJU_c00900 | CLRAG_19680 |
| 2283 | NAD(P)H-hydrate epimerase | | CAETHG_2416 | CLJU_c03010 | CLRAG_28620 |
| 2284 | Sugar phosphate isomerase/epimerase | | CAETHG_1474 | CLJU_c35660 | CLRAG_06190 |
| 2285 | glucuronide carrier protein | | CAETHG_0138 | CLJU_c20550 | CLRAG_19440 |
| 2286 | Phosphoglycerol transferase MdoB | | CAETHG_0576, | CLJU_c25080, | CLRAG_17690 |
| | | | CAETHG_2971 | CLJU_c08770 | |
| 2287 | Putative cell wall binding repeat 2 | | CAETHG_0972 | CLJU_c29730 | CLRAG_35730 |
| 2288 | Putative cell wall binding repeat 2 | 3.5.1.28 | CAETHG_2558, | CLJU_c04990 | CLRAG_38380 |
| | | | CAETHG_2576 | | |
| 2289 | Ig-like domain (group 4) | | CAETHG_2681 | CLJU_c05860 | CLRAG_07100 |
| 2290 | Ig-like domain (group 4) | | CAETHG_3096 | CLJU_c10060 | CLRAG_13310 |
| 2291 | Ig-like domain (group 4) | | CAETHG_3097 | CLJU_c10070 | CLRAG_13300 |
| 2292 | solute:Na+ symporter, SSS family | | CAETHG_0381 | CLJU_c23180 | CLRAG_01550 |
| 2293 | thiamine biosynthesis protein ThiI | | CAETHG_0832 | CLJU_c28350 | CLRAG_34430 |
| 2294 | thiamine biosynthesis lipoprotein | | CAETHG_1459 | CLJU_c35510 | CLRAG_06040 |
| 2295 | acyl-CoA thioester hydrolase | 3.1.2. | CAETHG_1524 | CLJU_c36140 | CLRAG_06660 |
| 2296 | Predicted transcriptional regulator YdeE, contains | | CAETHG_3624 | CLJU_c15220 | CLRAG_24130 |
| | AraC-type DNA-binding domain | | | | |
| 2297 | DNA gyrase inhibitor GyrI | | CAETHG_3857 | CLJU_c17440 | CLRAG_01270 |
| 2298 | transcription elongation factor GreA | | CAETHG_0720 | CLJU_c26390 | CLRAG_04480 |
| 2299 | transcription elongation factor GreA | | CAETHG_1453 | CLJU_c35450 | CLRAG_05980 |
| 2300 | transcription elongation factor GreA | | CAETHG_1983 | CLJU_c41490 | CLRAG_04620 |
| 2301 | Iron only hydrogenase large subunit, C-terminal | | CAETHG_0119 | CLJU_c20370 | CLRAG_25920 |
| | domain | | | | |
| 2302 | PucR C-terminal helix-turn-helix domain- | | CAETHG_0128 | CLJU_c20460 | CLRAG_19560 |
| | containing protein | | | | |
| 2303 | DNA-binding transcriptional regulator, PucR family | | CAETHG_0158 | CLJU_c20730 | CLRAG_19270 |
| 2304 | GntR family transcriptional regulator, | | CAETHG_0249 | CLJU_c21620 | CLRAG_31210 |
| | transcriptional repressor for pyruvate | | | | |
| | dehydrogenase complex | | | | |
| 2305 | putative transcriptional regulator | | CAETHG_0545, | CLJU_c24800, | CLRAG_17990 |
| | | | CAETHG_3487 | CLJU_c14060 | |
| 2306 | DNA-binding transcriptional regulator, PadR family | | CAETHG_0562 | CLJU_c24950 | CLRAG_17830 |
| 2307 | DNA-binding transcriptional regulator YhcF, GntR | | CAETHG_0581 | CLJU_c25130 | CLRAG_03470 |
| | family | | | | |
| 2308 | thiaminase (transcriptional activator TenA) | | CAETHG_0610 | CLJU_c25410 | CLRAG_03690 |
| 2309 | DNA-binding response regulator, OmpR family, | | CAETHG_0644, | CLJU_c38650, | CLRAG_20970 |
| | contains REC and winged-helix (wHTH) domain | | CAETHG_1715 | CLJU_c25750 | |
| 2310 | Helix-turn-helix domain-containing protein | | CAETHG_0746 | CLJU_c26650 | CLRAG_08520 |
| 2311 | looped-hinge helix DNA binding domain-containing | | CAETHG_0771 | CLJU_c26870 | CLRAG_16110 |
| | protein, AbrB family | | | | |
| 2312 | Response regulator receiver domain-containing | | CAETHG_0913 | CLJU_c29210 | CLRAG_35210 |
| | protein | | | | |
| 2313 | DNA-binding response regulator, OmpR family, | | CAETHG_1014, | CLJU_c13780, | CLRAG_15720 |
| | contains REC and winged-helix (wHTH) domain | | CAETHG_3461 | CLJU_c30140 | |
| 2314 | putative transcriptional regulator | | CAETHG_1179, | CLJU_c18490, | CLRAG_15520 |
| | | | CAETHG_3956 | CLJU_c32810 | |
| 2315 | DNA-binding transcriptional regulator, PadR family | | CAETHG_1191 | CLJU_c32930 | CLRAG_15400 |
| 2316 | DNA-binding transcriptional regulator, MocR | 2.6.1.23, | CAETHG_1287 | CLJU_c33890 | CLRAG_14040 |
| | family, contains an aminotransferase domain | 2.6.1.1 | | | |
| 2317 | DNA-binding transcriptional regulator, MerR family | | CAETHG_1416 | CLJU_c35070 | CLRAG_26330 |
| 2318 | GntR family transcriptional regulator | | CAETHG_1438 | CLJU_c35290 | CLRAG_05820 |
| 2319 | Predicted DNA-binding transcriptional regulator | | CAETHG_1466 | CLJU_c35580 | CLRAG_06110 |
| | YafY, contains an HTH and WYL domains | | | | |
| 2320 | DNA-binding transcriptional regulator, LysR family | | CAETHG_1627 | CLJU_c37800 | CLRAG_37220 |
| 2321 | central glycolytic genes regulator | | CAETHG_1761 | CLJU_c39160 | CLRAG_21310 |
| 2322 | GntR family transcriptional regulator, regulator for | 2.6.1.23, | CAETHG_1837 | CLJU_c39910 | CLRAG_22160 |
| | abcA and norABC | 2.6.1.1 | | | |
| 2323 | Helix-turn-helix | | CAETHG_2022 | CLJU_c41930 | CLRAG_05040 |
| 2324 | hypothetical protein | | CAETHG_2132 | CLJU_c00130 | CLRAG_20190 |
| 2325 | DNA-binding response regulator, OmpR family, | | CAETHG_2197 | CLJU_c00820 | CLRAG_19760 |
| | contains REC and winged-helix (wHTH) domain | | | | |
| 2326 | regulatory protein, Fis family | | CAETHG_2443 | CLJU_c03810 | CLRAG_28880 |
| 2327 | GntR family transcriptional regulator | | CAETHG_2535 | CLJU_c04630 | CLRAG_37990 |
| 2328 | Tetratricopeptide repeat-containing protein | | CAETHG_2714 | CLJU_c06150 | CLRAG_07430 |
| 2329 | Tetratricopeptide repeat-containing protein | | CAETHG_2716 | CLJU_c06160 | CLRAG_07440 |
| 2330 | DNA-binding response regulator, OmpR family, | | CAETHG_2747 | CLJU_c06510 | CLRAG_30390 |
| | contains REC and winged-helix (wHTH) domain | | | | |
| 2331 | Lrp/AsnC family transcriptional regulator, | | CAETHG_2764 | CLJU_c06730 | CLRAG_18530 |
| | regulator for asnA, asnC and gidA | | | | |
| 2332 | DNA-binding response regulator, OmpR family, | | CAETHG_2785 | CLJU_c06950 | CLRAG_18790 |
| | contains REC and winged-helix (wHTH) domain | | | | |
| 2333 | PucR C-terminal helix-turn-helix domain- | | CAETHG_2847 | CLJU_c07540 | CLRAG_32330 |
| | containing protein | | | | |
| 2334 | DNA-binding response regulator, OmpR family, | | CAETHG_2863 | CLJU_c07700 | CLRAG_25240 |
| | contains REC and winged-helix (wHTH) domain | | | | |
| 2335 | cAMP-binding domain of CRP or a regulatory | | CAETHG_2865 | CLJU_c07720 | CLRAG_25260 |
| | subunit of cAMP-dependent protein kinases | | | | |
| 2336 | CBS domain-containing protein | | CAETHG_2908 | CLJU_c08130 | CLRAG_08220 |
| 2337 | DNA-binding transcriptional regulator, MarR family | | CAETHG_2937 | CLJU_c08440 | CLRAG_08020 |
| 2338 | DNA-binding transcriptional regulator, MerR family | | CAETHG_2964 | CLJU_c08700 | CLRAG_07790 |
| 2339 | DNA-binding transcriptional regulator, MocR | 2.6.1.23, | CAETHG_2980 | CLJU_c08850 | CLRAG_07630 |
| | family, contains an aminotransferase domain | 2.6.1.1 | | | |
| 2340 | transcriptional repressor NrdR | | CAETHG_3317 | CLJU_c12350 | CLRAG_11450 |
| 2341 | two-component system, OmpR family, alkaline | | CAETHG_3319 | CLJU_c12370 | CLRAG_11430 |
| | phosphatase synthesis response regulator PhoP | | | | |
| 2342 | arsenate reductase | | CAETHG_3456 | CLJU_c13740 | CLRAG_10110 |
| 2343 | DNA-binding transcriptional regulator, MerR family | | CAETHG_3493 | CLJU_c14120 | CLRAG_09460 |
| 2344 | hypothetical protein | | CAETHG_3568 | CLJU_c14680 | CLRAG_20530 |
| 2345 | PucR C-terminal helix-turn-helix domain- | | CAETHG_3605 | CLJU_c15010 | CLRAG_24340 |
| | containing protein | | | | |
| 2346 | DNA-binding transcriptional regulator, MarR family | | CAETHG_3618 | CLJU_c15160 | CLRAG_24190 |
| 2347 | Predicted DNA-binding transcriptional regulator | | CAETHG_3623 | CLJU_c15210 | CLRAG_24140 |
| | YafY, contains an HTH and WYL domains | | | | |
| 2348 | DNA-binding transcriptional regulator, MerR family | | CAETHG_3652 | CLJU_c15510 | CLRAG_32590 |
| 2349 | putative transcriptional regulator | | CAETHG_3870 | CLJU_c17620 | CLRAG_01120 |
| 2350 | BlaI family transcriptional regulator, penicillinase | | CAETHG_4026 | CLJU_c18920 | CLRAG_40050 |
| | repressor | | | | |
| 2351 | DNA-binding transcriptional regulator, MerR family | | CAETHG_1860 | CLJU_c40110 | CLRAG_22340 |
| 2352 | Transcriptional regulator containing PAS, AAA-type | | CAETHG_1910 | CLJU_c40670 | CLRAG_22860 |
| | ATPase, and DNA-binding Fis domains | | | | |
| 2353 | AraC family transcriptional regulator | | CAETHG_2182 | CLJU_c00640 | CLRAG_19940 |
| 2354 | transcriptional regulator CtsR | | CAETHG_1977 | CLJU_c41360 | CLRAG_23550 |
| 2355 | transcriptional pleiotropic repressor | | CAETHG_3384 | CLJU_c13010 | CLRAG_10780 |
| 2356 | aspartate 4-decarboxylase | | CAETHG_1226 | CLJU_c33270 | CLRAG_14990 |
| 2357 | Transglycosylase SLT domain-containing protein | | CAETHG_1562 | CLJU_c37010 | CLRAG_36550 |
| 2358 | L-threonylcarbamoyladenylate synthase | | CAETHG_2334 | CLJU_c02290 | CLRAG_27890 |
| 2359 | translation initiation factor IF-3 | | CAETHG_0161, | CLJU_c34460, | CLRAG_19240 |
| | | | CAETHG_1346 | CLJU_c20760 | |
| 2360 | hypothetical protein | | CAETHG_2810 | CLJU_c07180 | CLRAG_26660 |
| 2361 | MFS transporter, DHA1 family, | | CAETHG_1485 | CLJU_c35770 | CLRAG_06300 |
| | bicyclomycin/chloramphenicol resistance protein | | | | |
| 2362 | Predicted arabinose efflux permease, MFS family | | CAETHG_3508 | CLJU_c14260 | CLRAG_09580 |
| 2363 | Predicted arabinose efflux permease, MFS family | | CAETHG_3509 | CLJU_c14270 | CLRAG_09590 |
| 2364 | ATP-binding cassette, subfamily B | | CAETHG_0699 | CLJU_c26230 | CLRAG_04350 |
| 2365 | ATP-binding cassette, subfamily B | | CAETHG_0700 | CLJU_c26240 | CLRAG_04360 |
| 2366 | Predicted arabinose efflux permease, MFS family | | CAETHG_2981 | CLJU_c08860 | CLRAG_07620 |
| 2367 | NitT/TauT family transport system substrate-binding protein | | CAETHG_0734 | CLJU_c26530 | CLRAG_08410 |
| 2368 | MFS transporter, putative metabolite:H+ | | CAETHG_0198 | CLJU_c21120 | CLRAG_30690 |
| | symporter | | | | |
| 2369 | MFS transporter, putative metabolite:H+ | | CAETHG_0200 | CLJU_c21140 | CLRAG_30700 |
| | symporter | | | | |
| 2370 | Major Facilitator Superfamily protein | | CAETHG_0565, | CLJU_c36100, | CLRAG_17800 |
| | | | CAETHG_1519 | CLJU_c24980 | |
| 2371 | drug resistance transporter, EmrB/QacA subfamily | | CAETHG_1177, | CLJU_c32790, | CLRAG_15570 |
| | | | CAETHG_1412 | CLJU_c35030 | |
| 2372 | ABC-2 family transporter protein | | CAETHG_1435, | CLJU_c35270, | CLRAG_05790 |
| | | | CAETHG_1436 | CLJU_c35260 | |
| 2373 | bacitracin transport system permease protein | | CAETHG_2193 | CLJU_c00780 | CLRAG_19800 |
| 2374 | Multidrug efflux pump subunit AcrB | | CAETHG_2472, | CLJU_c07600, | CLRAG_26960 |
| | | | CAETHG_2853 | CLJU_c04090 | |
| 2375 | drug resistance transporter, EmrB/QacA subfamily | | CAETHG_2505 | CLJU_c04370 | CLRAG_37700 |
| 2376 | Permease of the drug/metabolite transporter | | CAETHG_2728 | CLJU_c06330 | CLRAG_30500 |
| | (DMT) superfamily | | | | |
| 2377 | bacitracin transport system permease protein | | CAETHG_2743 | CLJU_c06470 | CLRAG_30430 |
| 2378 | Uncharacterized membrane-anchored protein YitT, | | CAETHG_2855 | CLJU_c07620 | CLRAG_25190 |
| | contains DUF161 and DUF2179 domains | | | | |
| 2379 | transposase | | CAETHG_3076 | CLJU_c09830, | CLRAG_16750 |
| | | | | CLJU_c05390 | |
| 2380 | Transposase InsO and inactivated derivatives | | CAETHG_4055 | CLJU_c19200 | CLRAG_37620 |
| 2381 | tRNA modification GTPase | | CAETHG_2118 | CLJU_c42920 | CLRAG_25730 |
| 2382 | tRNA(Ile)-lysidine synthase | | CAETHG_1989 | CLJU_c41550 | CLRAG_04680 |
| 2383 | twitching motility protein PilT | | CAETHG_3309 | CLJU_c12270 | CLRAG_11530 |
| 2384 | Y_Y_Y domain-containing protein | | CAETHG_1563 | CLJU_c37020 | CLRAG_36560 |
| 2385 | Signal transduction histidine kinase | | CAETHG_1843 | CLJU_c39970 | CLRAG_22210 |
| 2386 | Signal transduction histidine kinase | | CAETHG_3499 | CLJU_c14180 | CLRAG_09510 |
| 2387 | Signal transduction histidine kinase | | CAETHG_3690, | CLJU_c15810, | CLRAG_29260 |
| | | | CAETHG_3845 | CLJU_c17320 | |
| 2388 | Signal transduction histidine kinase | | CAETHG_0787 | CLJU_c27040 | CLRAG_08800 |
| 2389 | Signal transduction histidine kinase | | CAETHG_2746 | CLJU_c06500 | CLRAG_30400 |
| 2390 | hypothetical protein | | CAETHG_2864 | CLJU_c07710 | CLRAG_25250 |
| 2391 | DNA-binding response regulator, OmpR family, | | CAETHG_0022 | CLJU_c19450 | CLRAG_39660 |
| | contains REC and winged-helix (wHTH) domain | | | | |
| 2392 | two-component system, response regulator YcbB | | CAETHG_0162 | CLJU_c20770 | CLRAG_19230 |
| 2393 | DNA-binding response regulator, OmpR family, | | CAETHG_0357, | CLJU_c09170, | CLRAG_01840 |
| | contains REC and winged-helix (wHTH) domain | | CAETHG_3011 | CLJU_c22950 | |
| 2394 | DNA-binding response regulator, OmpR family, | | CAETHG_0578 | CLJU_c25100 | CLRAG_17670 |
| | contains REC and winged-helix (wHTH) domain | | | | |
| 2395 | DNA-binding response regulator, OmpR family, | | CAETHG_0641 | CLJU_c25720 | CLRAG_03920 |
| | contains REC and winged-helix (wHTH) domain | | | | |
| 2396 | DNA-binding response regulator, OmpR family, | | CAETHG_1067 | CLJU_c30630 | CLRAG_16060 |
| | contains REC and winged-helix (wHTH) domain | | | | |
| 2397 | arginine utilization regulatory protein | | CAETHG_1186 | CLJU_c32880 | CLRAG_15450 |
| 2398 | two-component system, OmpR family, KDP operon | | CAETHG_1796 | CLJU_c39510 | CLRAG_21710 |
| | response regulator KdpE | | | | |
| 2399 | DNA-binding response regulator, OmpR family, | | CAETHG_1844 | CLJU_c39980 | CLRAG_22220 |
| | contains REC and winged-helix (wHTH) domain | | | | |
| 2400 | DNA-binding response regulator, OmpR family, | | CAETHG_1873, | CLJU_c40270, | CLRAG_07660 |
| | contains REC and winged-helix (wHTH) domain | | CAETHG_2978 | CLJU_c08830 | |
| 2401 | DNA-binding response regulator, OmpR family, | | CAETHG_2516 | CLJU_c04440 | CLRAG_37800 |
| | contains REC and winged-helix (wHTH) domain | | | | |
| 2402 | DNA-binding response regulator, OmpR family, | | CAETHG_2875 | CLJU_c07820 | CLRAG_25280 |
| | contains REC and winged-helix (wHTH) domain | | | | |
| 2403 | DNA-binding response regulator, OmpR family, | | CAETHG_3601 | CLJU_c14970 | CLRAG_24380 |
| | contains REC and winged-helix (wHTH) domain | | | | |
| 2404 | DNA-binding response regulator, OmpR family, | | CAETHG_3689, | CLJU_c15800, | CLRAG_29270 |
| | contains REC and winged-helix (wHTH) domain | | CAETHG_3844 | CLJU_c17310 | |
| 2405 | DNA-binding response regulator, NarL/FixJ family, | | CAETHG_4017 | CLJU_c18850 | CLRAG_40150 |
| | contains REC and HTH domains | | | | |
| 2406 | Signal transduction histidine kinase | | CAETHG_0358 | CLJU_c22960 | CLRAG_01820 |
| 2407 | Signal transduction histidine kinase | | CAETHG_0636 | CLJU_c25670 | CLRAG_03870 |
| 2408 | Signal transduction histidine kinase | | CAETHG_0645 | CLJU_c25760 | CLRAG_03990 |
| 2409 | Sensor_kinase_SpoOB-type, alpha-helical domain | | CAETHG_0844, | CLJU_c10870 | CLRAG_03760 |
| | | | CAETHG_3177 | | |
| 2410 | two-component system, AgrA family, sensor | | CAETHG_1426 | CLJU_c35180 | CLRAG_05690 |
| | histidine kinase AgrC | | | | |
| 2411 | two-component system, LytT family, sensor kinase | | CAETHG_1589 | CLJU_c37330 | CLRAG_36770 |
| 2412 | Signal transduction histidine kinase | | CAETHG_2196 | CLJU_c00810 | CLRAG_19770 |
| 2413 | Signal transduction histidine kinase | | CAETHG_2877 | CLJU_c07840 | CLRAG_25320 |
| 2414 | Signal transduction histidine kinase | | CAETHG_3012 | CLJU_c09180 | CLRAG_13850 |
| 2415 | Signal transduction histidine kinase | | CAETHG_1194 | CLJU_c32960 | CLRAG_15370 |
| 2416 | Histidine kinase-, DNA gyrase B-, and HSP90-like | | CAETHG_1814 | CLJU_c39680 | CLRAG_21930 |
| | ATPase | | | | |
| 2417 | tight adherence protein C | | CAETHG_0874 | CLJU_c28790 | CLRAG_34870 |
| 2418 | tight adherence protein B | | CAETHG_0875 | CLJU_c28800 | CLRAG_34880 |
| 2419 | pilus assembly protein CpaF | | CAETHG_0876 | CLJU_c28810 | CLRAG_34890 |
| 2420 | leader peptidase (prepilin peptidase)/N- | | CAETHG_0885 | CLJU_c28890 | CLRAG_34970 |
| | methyltransferase | | | | |
| 2421 | ubiquinone biosynthesis protein | | CAETHG_2190 | CLJU_c00730 | CLRAG_19850 |
| 2422 | hypothetical protein | | CAETHG_1306 | CLJU_c34080 | CLRAG_14230 |
| 2423 | UDP-N-acetyl-D-glucosamine dehydrogenase | 1.1.1.22 | CAETHG_1308 | CLJU_c34100 | CLRAG_14250 |
| 2424 | N-acetylglucosaminyldiphosphoundecaprenol N- | 2.4.1.187 | CAETHG_1299 | CLJU_c34010 | CLRAG_14160 |
| | acetyl-beta-D-mannosaminyltransferase | | | | |
| 2425 | UDP-N-acetylmuramyl tripeptide synthase | | CAETHG_2720 | CLJU_c06200 | CLRAG_07480 |
| 2426 | UDP-GlcNAc:undecaprenyl-phosphate GlcNAc-1- | 2.7.8.— | CAETHG_2340 | CLJU_c02340 | CLRAG_27950 |
| | phosphate transferase | | | | |
| 2427 | Nucleotide-binding universal stress protein, UspA | | CAETHG_1201, | CLJU_c37680, | CLRAG_15300 |
| | family | | CAETHG_1622 | CLJU_c33030 | |
| 2428 | DNA polymerase | | CAETHG_2223 | CLJU_c01120 | CLRAG_30280 |
| 2429 | uracil permease | | CAETHG_0422 | CLJU_c23580 | CLRAG_17590 |
| 2430 | nucleobase:cation symporter-1, NCS1 family | | CAETHG_1497 | CLJU_c35900 | CLRAG_06400 |
| 2431 | uracil permease | | CAETHG_3163 | CLJU_c10730 | CLRAG_12610 |
| 2432 | uroporphyrinogen decarboxylase | | CAETHG_0192 | CLJU_c21070 | CLRAG_30650 |
| 2433 | Uroporphyrinogen-III decarboxylase | | CAETHG_0141 | CLJU_c20580 | CLRAG_19400 |
| 2434 | Uroporphyrinogen decarboxylase (URO-D) | | CAETHG_0151, | CLJU_c20680, | CLRAG_19290 |
| | | | CAETHG_0155 | CLJU_c20710 | |
| 2435 | uroporphyrinogen decarboxylase | | CAETHG_0564, | CLJU_c24990, | CLRAG_17790 |
| | | | CAETHG_0566 | CLJU_c24970 | |
| 2436 | uroporphyrinogen decarboxylase | | CAETHG_1370 | CLJU_c27560, | CLRAG_30730 |
| | | | | CLJU_c34730 | |
| 2437 | Protein-arginine kinase activator protein McsA | | CAETHG_1976 | CLJU_c41350 | CLRAG_23540 |
| 2438 | Vancomycin resistance protein YoaR, contains | | CAETHG_1249, | CLJU_c18940, | CLRAG_24780 |
| | peptidoglycan-binding and VanW domains | | CAETHG_4028 | CLJU_c33500 | |
| 2439 | Vancomycin resistance protein YoaR, contains | | CAETHG_1769 | CLJU_c39250 | CLRAG_21460 |
| | peptidoglycan-binding and VanW domains | | | | |
| 2440 | putative peptidoglycan lipid II flippase | | CAETHG_1310 | CLJU_c34120 | CLRAG_14270 |
| 2441 | H+/Cl− antiporter ClcA | | CAETHG_0387 | CLJU_c23240 | CLRAG_01490 |
| 2442 | xanthine dehydrogenase accessory factor | | CAETHG_2768 | CLJU_c06770 | CLRAG_18570 |
| 2443 | selenium-dependent xanthine dehydrogenase | | CAETHG_0457 | CLJU_c23910 | CLRAG_17250 |
| 2444 | CO or xanthine dehydrogenase, FAD-binding | | CAETHG_0454 | CLJU_c23880 | CLRAG_17280 |
| | subunit | | | | |
| 2445 | CO or xanthine dehydrogenase, FAD-binding | 1.1.1.204, | CAETHG_0991 | CLJU_c29920 | CLRAG_35920 |
| | subunit | 1.17.1.4 | | | |
| 2446 | xanthine phosphoribosyltransferase | 2.4.2.22, | CAETHG_3614 | CLJU_c15120 | CLRAG_24220 |
| | | 2.4.2.8 | | | |
| 2447 | xanthine permease | | CAETHG_0450 | CLJU_c23850 | CLRAG_17320 |
| 2448 | nucleobase:cation symporter-2, NCS2 family | | CAETHG_3615 | CLJU_c15130 | CLRAG_24210 |
| 2449 | XTP/dITP diphosphohydrolase | | CAETHG_1546 | CLJU_c36850 | CLRAG_36380 |
| 2450 | Sugar kinase of the NBD/HSP70 family, may | | CAETHG_3936 | CLJU_c18290 | CLRAG_00330 |
| | contain an N-terminal HTH domain | | | | |
| 2451 | hypothetical protein | | CAETHG_2502 | CLJU_c04340 | CLRAG_37670 |
| 2452 | replication restart DNA helicase PriA | | CAETHG_2985 | CLJU_c08900 | CLRAG_07580 |
| 2453 | Predicted Zn-dependent peptidase | | CAETHG_3017 | CLJU_c09230 | CLRAG_13800 |
| 2454 | zinc transporter, ZIP family | | CAETHG_2333 | CLJU_c02280 | CLRAG_27880 |
| 2455 | (R,R)-butanediol dehydrogenase/meso- | 1.1.1.4, | CAETHG_0385 | CLJU_c23220 | CLRAG_01510 |
| | butanediol dehydrogenase/diacetyl reductase | 1.1.1.1 | | | |
| 2456 | hypothetical protein | | CAETHG_3340 | CLJU_c12580 | CLRAG_11220 |
| 2457 | Predicted Zn-dependent peptidase | | CAETHG_1449 | CLJU_c35410 | CLRAG_05950 |
| 2458 | Predicted Zn-dependent peptidase | | CAETHG_3405 | CLJU_c13220 | CLRAG_10570 |
| 2459 | Glyoxylase, beta-lactamase superfamily II | 3.1.2.6 | CAETHG_1267 | CLJU_c33690 | CLRAG_24590 |
| 2460 | Glyoxylase, beta-lactamase superfamily II | | CAETHG_2957 | CLJU_c08630 | CLRAG_07860 |
| 2461 | hypothetical protein | | CAETHG_3638 | CLJU_c15360 | CLRAG_24100 |
| 2462 | putrescine carbamoyltransferase | | CAETHG_2082 | CLJU_c42560 | CLRAG_09040 |
| 2463 | pyrimidine-nucleoside phosphorylase | 2.4.2.23, | CAETHG_3925 | CLJU_c18160 | CLRAG_00510 |
| | | 2.4.2.2, | | | |
| | | 2.4.2.3, | | | |
| | | 2.4.2.4, | | | |
| | | 2.4.2.1 | | | |
| 2464 | pyrroline-5-carboxylate reductase | 1.5.1.2 | CAETHG_1593 | CLJU_c37380 | CLRAG_36820 |
| 2465 | pyruvate carboxylase | | CAETHG_1594 | CLJU_c37390 | CLRAG_36830 |
| 2466 | formate C-acetyltransferase | 2.3.1.54 | CAETHG_0667 | CLJU_c25980 | CLRAG_04120 |
| 2467 | formate C-acetyltransferase | 2.3.1.54 | CAETHG_1829 | CLJU_c39830 | CLRAG_22080 |
| 2468 | pyruvate formate lyase activating enzyme | 2.3.1.54 | CAETHG_0666 | CLJU_c25970 | CLRAG_04110 |
| 2469 | pyruvate formate lyase activating enzyme | 2.3.1.54 | CAETHG_1828 | CLJU_c39820 | CLRAG_22070 |
| 2470 | pyruvate kinase | 2.7.1.40 | CAETHG_2441 | CLJU_c03260 | CLRAG_28860 |
| 2471 | pyruvate phosphate dikinase | 2.7.9.1 | CAETHG_2909 | CLJU_c08140 | CLRAG_08210 |
| 2472 | putative pyruvate formate lyase activating enzyme | | CAETHG_2021 | CLJU_c41920 | CLRAG_05030 |
| 2473 | pyruvate-ferredoxin/flavodoxin oxidoreductase | 1.2.7.1 | CAETHG_0928, | CLJU_c09340, | CLRAG_35360 |
| | | | CAETHG_3029 | CLJU_c29340 | |
| 2474 | queuine tRNA-ribosyltransferase | | CAETHG_1278 | CLJU_c33800 | CLRAG_24480 |
| 2475 | quinolinate synthetase | | CAETHG_0503 | CLJU_c24430 | CLRAG_25160 |
| 2476 | biotin synthase | 2.8.1.6 | CAETHG_0339 | CLJU_c22770 | CLRAG_02020 |
| 2477 | recombination protein RecA | | CAETHG_3411 | CLJU_c13280 | CLRAG_10510 |
| 2478 | replication restart DNA helicase PriA | | CAETHG_3337 | CLJU_c12550 | CLRAG_11250 |
| 2479 | diguanylate cyclase (GGDEF) domain-containing | | CAETHG_3590, | CLJU_c15740, | CLRAG_20280 |
| | protein | | CAETHG_3682 | CLJU_c14840 | |
| 2480 | 3,4-dihydroxy 2-butanone 4-phosphate synthase/ | 3.5.4.25 | CAETHG_0305 | CLJU_c22070 | CLRAG_31590 |
| | GTP cyclohydrolase II | | | | |
| 2481 | riboflavin synthase alpha chain | 2.5.1.9 | CAETHG_0306 | CLJU_c22080 | CLRAG_31600 |
| 2482 | ribonuclease P protein component | | CAETHG_2122 | CLJU_c42960 | CLRAG_25770 |
| 2483 | ribonuclease PH | | CAETHG_1547 | CLJU_c36860 | CLRAG_36390 |
| 2484 | ribonucleoside-diphosphate reductase class II | | CAETHG_2775 | CLJU_c06840 | CLRAG_18680 |
| 2485 | ribonucleoside-triphosphate reductase class III | 1.17.4.2 | CAETHG_2287 | CLJU_c01840 | CLRAG_27440 |
| | catalytic subunit | | | | |
| 2486 | hypothetical protein | | CAETHG_2324 | CLJU_c02190 | CLRAG_27790 |
| 2487 | ribose ABC transporter membrane protein | | CAETHG_2237 | CLJU_c01280 | CLRAG_30140 |
| 2488 | ribose-5-phosphate isomerase | 5.3.1.6 | CAETHG_2336 | CLJU_c02310 | CLRAG_27910 |
| 2489 | ribose transport system substrate-binding protein | | CAETHG_2235 | CLJU_c01260 | CLRAG_30160 |
| 2490 | ribose-phosphate pyrophosphokinase | 2.7.6.1 | CAETHG_2006 | CLJU_c41770 | CLRAG_04900 |
| 2491 | 23S rRNA pseudouridine2605 synthase | 4.2.1.70 | CAETHG_0222 | CLJU_c21360 | CLRAG_30920 |
| 2492 | 23S rRNA pseudouridine1911/1915/1917 synthase | | CAETHG_2835 | CLJU_c07430 | CLRAG_32260 |
| 2493 | 23S rRNA pseudouridine1911/1915/1917 synthase | 4.2.1.70 | CAETHG_3162 | CLJU_c10720 | CLRAG_12620 |
| 2494 | ribosomal protein L11 methyltransferase | | CAETHG_2893 | CLJU_c08000 | CLRAG_08360 |
| 2495 | large subunit ribosomal protein L19 | | CAETHG_3375 | CLJU_c12930 | CLRAG_10870 |
| 2496 | large subunit ribosomal protein L7A | | CAETHG_1953 | CLJU_c41100 | CLRAG_23290 |
| 2497 | 16S rRNA pseudouridine516 synthase | 4.2.1.70 | CAETHG_1561 | CLJU_c37000 | CLRAG_36540 |
| 2498 | ribosome-binding factor A | | CAETHG_3399 | CLJU_c13160 | CLRAG_10630 |
| 2499 | Sugar (pentulose or hexulose) kinase | 2.7.1.47, | CAETHG_2230 | CLJU_c01220 | CLRAG_30200 |
| | | 2.7.1.16 | | | |
| 2500 | ribulose-phosphate 3-epimerase | 5.1.3.1 | CAETHG_3346 | CLJU_c12640 | CLRAG_11160 |
| 2501 | RNA polymerase sigma-70 factor, ECF subfamily | | CAETHG_0987 | CLJU_c29880 | CLRAG_35890 |
| 2502 | RNA polymerase sporulation-specific sigma factor | | CAETHG_1293 | CLJU_c33950 | CLRAG_14100 |
| 2503 | RNA polymerase sporulation-specific sigma factor | | CAETHG_3308 | CLJU_c12260 | CLRAG_11540 |
| 2504 | RNA polymerase, sigma 28 subunit, | | CAETHG_3131 | CLJU_c10410 | CLRAG_12960 |
| | SigD/FliA/WhiG | | | | |
| 2505 | RNA polymerase, sigma 29 subunit, SigE | | CAETHG_3314 | CLJU_c12320 | CLRAG_11480 |
| 2506 | RNA polymerase sporulation-specific sigma factor | | CAETHG_1964 | CLJU_c41230 | CLRAG_23420 |
| 2507 | RNA polymerase, sigma 54 subunit, RpoN/SigL | | CAETHG_1762 | CLJU_c39170 | CLRAG_21320 |
| 2508 | RNA polymerase, sigma 70 subunit, RpoD | | CAETHG_2917 | CLJU_c08220 | CLRAG_08170 |
| 2509 | RNA polymerase, sigma subunit, RpsG/SigG | | CAETHG_3315 | CLJU_c12330 | CLRAG_11470 |
| 2510 | host factor-I protein | | CAETHG_0207 | CLJU_c21210 | CLRAG_30790 |
| 2511 | RNase HII | | CAETHG_3378 | CLJU_c12960 | CLRAG_10840 |
| 2512 | ribonuclease-3 | | CAETHG_3364 | CLJU_c12830 | CLRAG_10980 |
| 2513 | ribonuclease Z | | CAETHG_0745 | CLJU_c26640 | CLRAG_08510 |
| 2514 | rod shape-determining protein MreB | | CAETHG_2356, | CLJU_c07220, | CLRAG_28110 |
| | | | CAETHG_2814 | CLJU_c02500 | |
| 2515 | 23S rRNA (uracill939-C5)-methyltransferase | | CAETHG_2442 | CLJU_c03270 | CLRAG_28870 |
| 2516 | S-adenosylmethionine:tRNA ribosyltransferase- | | CAETHG_1279 | CLJU_c33810 | CLRAG_24470 |
| | isomerase | | | | |
| 2517 | S-ribosylhomocysteine lyase/quorum-sensing | | CAETHG_0412 | CLJU_c23480 | CLRAG_17630 |
| | autoinducer 2 (AI-2) synthesis protein LuxS | | | | |
| 2518 | RND family efflux transporter, MFP subunit | | CAETHG_2507 | CLJU_c04390 | CLRAG_37720 |
| 2519 | selenocysteine-specific elongation factor | | CAETHG_2840 | CLJU_c07480, | CLRAG_32210 |
| | | | | CLJU_c27700 | |
| 2520 | selenophosphate synthase | 2.7.9.3 | CAETHG_2838 | CLJU_c07460, | CLRAG_32230 |
| | | | | CLJU_c27720 | |
| 2521 | two-component system, sensor histidine kinase | | CAETHG_0163 | CLJU_c20780 | CLRAG_19220 |
| | YcbA | | | | |
| 2522 | septum site-determining protein MinC | | CAETHG_2818 | CLJU_c07260 | CLRAG_26580 |
| 2523 | serine O-acetyltransferase | 2.3.1.31, | CAETHG_1775 | CLJU_c39300 | CLRAG_21510 |
| | | 2.3.1.30 | | | |
| 2524 | glycine hydroxymethyltransferase | 2.1.2.1 | CAETHG_3241 | CLJU_c11500 | CLRAG_11950 |
| 2525 | serine/threonine protein kinase | | CAETHG_3344 | CLJU_c12620 | CLRAG_11180 |
| 2526 | seryl-tRNA synthetase | | CAETHG_2137 | CLJU_c00170 | CLRAG_20150 |
| 2527 | shikimate dehydrogenase | 1.1.1.282, | CAETHG_0904 | CLJU_c29120 | CLRAG_35120 |
| | | 1.1.1.25 | | | |
| 2528 | shikimate kinase | 2.7.1.71 | CAETHG_0903 | CLJU_c29110 | CLRAG_35110 |
| 2529 | Predicted kinase | | CAETHG_3445 | CLJU_c13630 | CLRAG_10240 |
| 2530 | Transcriptional regulator containing PAS, AAA-type | | CAETHG_0105, | CLJU_c20240, | CLRAG_17190 |
| | ATPase, and DNA-binding Fis domains | | CAETHG_0463 | CLJU_c23970 | |
| 2531 | signal peptidase I | | CAETHG_2696 | CLJU_c05980 | CLRAG_07210 |
| 2532 | signal peptidase I | | CAETHG_3376 | CLJU_c12940 | CLRAG_10860 |
| 2533 | signal recognition particle subunit FFH/SRP54 | | CAETHG_3370 | CLJU_c12880 | CLRAG_10920 |
| | (srp54) | | | | |
| 2534 | fused signal recognition particle receptor | | CAETHG_3368 | CLJU_c12860 | CLRAG_10940 |
| 2535 | PAS domain S-box-containing protein | | CAETHG_0552, | CLJU_c24870, | CLRAG_17930 |
| | | | CAETHG_0554 | CLJU_c24850 | |
| 2536 | single-strand binding protein | | CAETHG_2104 | CLJU_c42780 | CLRAG_25590 |
| 2537 | single-strand DNA-binding protein | | CAETHG_3105 | CLJU_c10150 | CLRAG_13220 |
| 2538 | sirohydrochlorin cobaltochelatase | 4.99.1.3 | CAETHG_1113 | CLJU_c31850 | CLRAG_02480 |
| 2539 | Small basic protein | | CAETHG_3153 | CLJU_c10630 | CLRAG_12710 |
| 2540 | sodium/proton-potassium antiporter GerN, CPA2 | | CAETHG_2991 | CLJU_c08970 | CLRAG_07530 |
| | family | | | | |
| 2541 | spermidine synthase | 2.5.1.16 | CAETHG_0820 | CLJU_c28200 | CLRAG_09150 |
| 2542 | Regulator of protease activity HflC, | | CAETHG_2784 | CLJU_c06940 | CLRAG_18780 |
| | stomatin/prohibitin superfamily | | | | |
| 2543 | two-component system, response regulator, stage | | CAETHG_3212 | CLJU_c11220 | CLRAG_12240 |
| | 0 sporulation protein A | | | | |
| 2544 | anti-sigma B factor antagonist | | CAETHG_2401 | CLJU_c02860 | CLRAG_28460 |
| 2545 | stage II sporulation protein D | | CAETHG_0618 | CLJU_c25490 | CLRAG_03710 |
| 2546 | N-acetylmuramoyl-L-alanine amidase | | CAETHG_2413 | CLJU_c02980 | CLRAG_28590 |
| 2547 | spore germination protein KA | | CAETHG_1745 | CLJU_c38970 | CLRAG_21150 |
| 2548 | spore germination protein KB | | CAETHG_1747 | CLJU_c38990 | CLRAG_21170 |
| 2549 | spore germination protein KC | | CAETHG_1746 | CLJU_c38980 | CLRAG_21160 |
| 2550 | putative sporulation protein YtaF | | CAETHG_2680 | CLJU_c05850 | CLRAG_07090 |
| 2551 | SSU ribosomal protein S12P methylthiotransferase | | CAETHG_3409 | CLJU_c13260 | CLRAG_10530 |
| 2552 | SSU ribosomal protein S14P | | CAETHG_1934 | CLJU_c40910 | CLRAG_23100 |
| 2553 | SSU ribosomal protein S15P | | CAETHG_3403 | CLJU_c13200 | CLRAG_10590 |
| 2554 | small subunit ribosomal protein S16 | | CAETHG_3371 | CLJU_c12890 | CLRAG_10910 |
| 2555 | SSU ribosomal protein S17P | | CAETHG_1938 | CLJU_c40950 | CLRAG_23140 |
| 2556 | SSU ribosomal protein S18P | | CAETHG_2103 | CLJU_c42770 | CLRAG_25580 |
| 2557 | small subunit ribosomal protein S20 | | CAETHG_2883 | CLJU_c07900 | CLRAG_25380 |
| 2558 | small subunit ribosomal protein S2 | | CAETHG_3385 | CLJU_c13020 | CLRAG_10770 |
| 2559 | putative sigma-54 modulation protein | | CAETHG_2363 | CLJU_c02620 | CLRAG_28220 |
| 2560 | small subunit ribosomal protein S9 | | CAETHG_1913 | CLJU_c40700 | CLRAG_22890 |
| 2561 | putative DeoR family transcriptional regulator, | | CAETHG_2355 | CLJU_c02490 | CLRAG_28100 |
| | stage III sporulation protein D | | | | |
| 2562 | stage IV sporulation protein A | | CAETHG_3331 | CLJU_c12490 | CLRAG_11310 |
| 2563 | stage V sporulation protein S | | CAETHG_3413 | CLJU_c13300 | CLRAG_10490 |
| 2564 | Serine protease, subtilisin family | | CAETHG_3433 | CLJU_c13490, | CLRAG_10300 |
| | | | | CLJU_c13560 | |
| 2565 | succinate dehydrogenase/fumarate reductase | 1.3.99.1 | CAETHG_0342 | CLJU_c22800 | CLRAG_01990 |
| | flavoprotein subunit | | | | |
| 2566 | succinyl-diaminopimelate desuccinylase | | CAETHG_3852 | CLJU_c17390 | CLRAG_01300 |
| 2567 | MFS transporter, sugar porter (SP) family | | CAETHG_3935 | CLJU_c18280 | CLRAG_00340 |
| 2568 | TadE-like protein | | CAETHG_0881 | CLJU_c28850 | CLRAG_34930 |
| 2569 | 2-hydroxy-3-oxopropionate reductase | 1.1.1.60 | CAETHG_2186 | CLJU_c28090, | CLRAG_19890 |
| | | | | CLJU_c00680 | |
| 2570 | thiamine pyrophosphokinase | 2.7.6.2 | CAETHG_3347 | CLJU_c12650 | CLRAG_11150 |
| 2571 | thiamine-phosphate diphosphorylase | 2.5.1.3 | CAETHG_1204 | CLJU_c33060 | CLRAG_15270 |
| 2572 | thiamine-phosphate pyrophosphorylase | 2.5.1.3 | CAETHG_3428 | CLJU_c13440 | CLRAG_10350 |
| 2573 | Predicted thioesterase | 3.1.2. | CAETHG_1780 | CLJU_c39350 | CLRAG_21560 |
| 2574 | thioredoxin | | CAETHG_1893 | CLJU_c40500 | CLRAG_22690 |
| 2575 | thioredoxin reductase (NADPH) | 1.6.4.5, | CAETHG_1892 | CLJU_c40490 | CLRAG_22680 |
| | | 1.8.1.9 | | | |
| 2576 | threonine dehydratase | 4.3.1.19 | CAETHG_3611 | CLJU_c15090 | CLRAG_24280 |
| 2577 | threonine kinase | | CAETHG_1111 | CLJU_c31830 | CLRAG_02460 |
| 2578 | threonine synthase | 4.2.3.1, | CAETHG_1217 | CLJU_c33180 | CLRAG_15110 |
| | | 4.2.99.2 | | | |
| 2579 | threonyl-tRNA synthetase | | CAETHG_1347 | CLJU_c34470 | CLRAG_14550 |
| 2580 | dTMP kinase | 2.7.4.12, | CAETHG_2245 | CLJU_c01390 | CLRAG_27050 |
| | | 2.7.4.9 | | | |
| 2581 | Tetratricopeptide repeat-containing protein | | CAETHG_1319 | CLJU_c34200 | CLRAG_14350 |
| 2582 | Tetratricopeptide repeat-containing protein | | CAETHG_1525 | CLJU_c36150 | CLRAG_06670 |
| 2583 | Tetratricopeptide repeat-containing protein | | CAETHG_1894 | CLJU_c40510 | CLRAG_22700 |
| 2584 | TPR repeat-containing protein | | CAETHG_1897 | CLJU_c40540 | CLRAG_22730 |
| 2585 | fructose-6-phosphate aldolase, TalC/MipB family | 2.2.1.2 | CAETHG_0665 | CLJU_c25960 | CLRAG_04100 |
| 2586 | transaldolase | 2.2.1.2 | CAETHG_1810 | CLJU_c39640 | CLRAG_21880 |
| 2587 | transcription antitermination protein nusG | | CAETHG_1960 | CLJU_c41170 | CLRAG_23360 |
| 2588 | transcription termination factor Rho | | CAETHG_2327 | CLJU_c02220 | CLRAG_27820 |
| 2589 | transcription-repair coupling factor | | CAETHG_2001 | CLJU_c41720 | CLRAG_04850 |
| 2590 | uncharacterized protein | | CAETHG_2369 | CLJU_c02680 | CLRAG_28280 |
| 2591 | DNA-binding transcriptional regulator LsrR, DeoR | | CAETHG_0480 | CLJU_c24220 | CLRAG_24900 |
| | family | | | | |
| 2592 | BlaI family transcriptional regulator, penicillinase | | CAETHG_0584 | CLJU_c25160 | CLRAG_03500 |
| | repressor | | | | |
| 2593 | DNA-binding transcriptional regulator, MerR family | | CAETHG_0633 | CLJU_c25640 | CLRAG_03840 |
| 2594 | Lrp/AsnC family transcriptional regulator, leucine- | | CAETHG_0697 | CLJU_c26210 | CLRAG_04290 |
| | responsive regulatory protein | | | | |
| 2595 | MerR HTH family regulatory protein | | CAETHG_0758 | CLJU_c26770 | CLRAG_08640 |
| 2596 | DNA-binding transcriptional regulator, PadR family | | CAETHG_0778 | CLJU_c26940 | CLRAG_08790 |
| 2597 | Sigma-54 interaction domain-containing protein | | CAETHG_1556 | CLJU_c36950 | CLRAG_36490 |
| 2598 | redox-sensing transcriptional repressor | | CAETHG_1581 | CLJU_c37250 | CLRAG_36690 |
| 2599 | DNA-binding transcriptional regulator, LysR family | | CAETHG_1782 | CLJU_c39370 | CLRAG_21580 |
| 2600 | Helix-turn-helix | | CAETHG_1862 | CLJU_c40130 | CLRAG_22360 |
| 2601 | mRNA interferase MazF | | CAETHG_2419 | CLJU_c03040 | CLRAG_28650 |
| 2602 | Lrp/AsnC family transcriptional regulator, leucine- | | CAETHG_2477 | CLJU_c04150 | CLRAG_26910 |
| | responsive regulatory protein | | | | |
| 2603 | AraC-type DNA-binding protein | | CAETHG_2497 | CLJU_c04290 | CLRAG_26770 |
| 2604 | DNA-binding response regulator, OmpR family, | | CAETHG_2531, | CLJU_c04590, | CLRAG_37950 |
| | contains REC and winged-helix (wHTH) domain | | CAETHG_2867 | CLJU_c07750 | |
| 2605 | Transcriptional regulator, contains XRE-family HTH | | CAETHG_3453 | CLJU_c13710 | CLRAG_10130 |
| | domain | | | | |
| 2606 | AbrB family transcriptional regulator, stage V | | CAETHG_1999 | CLJU_c41700 | CLRAG_04830 |
| | sporulation protein T | | | | |
| 2607 | AraC-type DNA-binding protein | | CAETHG_0926 | CLJU_c29330 | CLRAG_35340 |
| 2608 | transcriptional regulator, AraC family | | CAETHG_1193 | CLJU_c32950 | CLRAG_15380 |
| 2609 | transcriptional regulator, AraC family | | CAETHG_1411 | CLJU_c35020 | CLRAG_26280 |
| 2610 | AraC-type DNA-binding protein | | CAETHG_3626 | CLJU_c15240 | CLRAG_24110 |
| 2611 | transcriptional regulator, ArgR family | | CAETHG_3019, | CLJU_c11190, | CLRAG_13780 |
| | | | CAETHG_3208 | CLJU_c09250 | |
| 2612 | ArsR family transcriptional regulator | | CAETHG_0267 | CLJU_c21790 | CLRAG_31330 |
| 2613 | transcriptional regulator, ArsR family | | CAETHG_0947 | CLJU_c29530 | CLRAG_35520 |
| 2614 | DNA-binding transcriptional regulator, ArsR family | | CAETHG_2289 | CLJU_c01860 | CLRAG_27460 |
| 2615 | transcriptional regulator, ArsR family | | CAETHG_3663 | CLJU_c15640 | CLRAG_32690 |
| 2616 | Lrp/AsnC family transcriptional regulator, leucine- | | CAETHG_0190 | CLJU_c21050 | CLRAG_30630 |
| | responsive regulatory protein | | | | |
| 2617 | transcriptional regulator, BadM/Rrf2 family | | CAETHG_1298 | CLJU_c34000 | CLRAG_14150 |
| 2618 | transcriptional regulator, BadM/Rrf2 family | | CAETHG_3292 | CLJU_c12100 | CLRAG_11700 |
| 2619 | transcriptional regulator, DeoR family | | CAETHG_0144 | CLJU_c20610 | CLRAG_19370 |
| 2620 | transcriptional regulator, DeoR family | | CAETHG_0677 | CLJU_c26080 | CLRAG_04170 |
| 2621 | transcriptional regulator, DeoR family | | CAETHG_3685 | CLJU_c15770 | CLRAG_32930 |
| 2622 | DNA-binding transcriptional regulator, MocR | 2.6.1.23, | CAETHG_0037 | CLJU_c19600 | CLRAG_39520 |
| | family, contains an aminotransferase domain | 2.6.1.1 | | | |
| 2623 | DNA-binding transcriptional regulator, MocR | 2.6.1.23, | CAETHG_1134 | CLJU_c32060 | CLRAG_02690 |
| | family, contains an aminotransferase domain | 2.6.1.1 | | | |
| 2624 | DNA-binding transcriptional regulator YhcF, GntR | | CAETHG_1908 | CLJU_c40650 | CLRAG_22840 |
| | family | | | | |
| 2625 | GntR family transcriptional regulator, arabinose | | CAETHG_2231 | CLJU_c01230 | CLRAG_30190 |
| | operon transcriptional repressor | | | | |
| 2626 | GntR family transcriptional regulator | | CAETHG_2767 | CLJU_c06760 | CLRAG_18560 |
| 2627 | GntR family transcriptional regulator, | | CAETHG_3474 | CLJU_c13910 | CLRAG_09220 |
| | transcriptional repressor for pyruvate | | | | |
| | dehydrogenase complex | | | | |
| 2628 | regulatory protein, gntR family | | CAETHG_3915 | CLJU_c18060 | CLRAG_00590 |
| 2629 | transcriptional regulator, HxlR family | | CAETHG_0486 | CLJU_c37760, | CLRAG_24950 |
| | | | | CLJU_c24280 | |
| 2630 | transcriptional regulator, HxlR family | | CAETHG_3884 | CLJU_c17760 | CLRAG_00950 |
| 2631 | transcriptional regulator, IclR family | | CAETHG_0932 | CLJU_c29380 | CLRAG_35380 |
| 2632 | transcriptional regulator, IclR family | | CAETHG_2177 | CLJU_c00590 | CLRAG_19990 |
| 2633 | transcriptional regulator, IclR family | | CAETHG_3442 | CLJU_c13600 | CLRAG_10270 |
| 2634 | transcriptional regulator, LacI family | | CAETHG_2293 | CLJU_c01900 | CLRAG_27500 |
| 2635 | DNA-binding transcriptional regulator, LysR family | | CAETHG_0059 | CLJU_c19790 | CLRAG_39340 |
| 2636 | DNA-binding transcriptional regulator, LysR family | | CAETHG_1229 | CLJU_c33300 | CLRAG_14960 |
| 2637 | transcriptional regulator, LysR family | | CAETHG_1742 | CLJU_c38940 | CLRAG_21120 |
| 2638 | transcriptional regulator, LytTR family | | CAETHG_0532 | CLJU_c24670 | CLRAG_18160 |
| 2639 | transcriptional regulator, LytTR family | | CAETHG_0727 | CLJU_c26460 | CLRAG_04550 |
| 2640 | DNA-binding transcriptional regulator, MarR family | | CAETHG_0345 | CLJU_c22830 | CLRAG_01960 |
| 2641 | transcriptional regulator, MarR family | | CAETHG_2051 | CLJU_c42200 | CLRAG_05310 |
| 2642 | DNA-binding transcriptional regulator, MarR family | | CAETHG_2541 | CLJU_c04690 | CLRAG_38070 |
| 2643 | transcriptional regulator, MarR family | | CAETHG_3646 | CLJU_c15450 | CLRAG_32580 |
| 2644 | transcriptional regulator, MarR family | | CAETHG_4023 | CLJU_c18890 | CLRAG_40080 |
| 2645 | DNA-binding transcriptional regulator, MerR family | | CAETHG_3243 | CLJU_c11520 | CLRAG_11930 |
| 2646 | poly-beta-hydroxybutyrate-responsive repressor | | CAETHG_0340 | CLJU_c22780 | CLRAG_02010 |
| 2647 | DNA-binding transcriptional regulator, PadR family | | CAETHG_0551 | CLJU_c24840 | CLRAG_17940 |
| 2648 | DNA-binding transcriptional regulator, PadR family | | CAETHG_0984 | CLJU_c29850 | CLRAG_35860 |
| 2649 | DNA-binding transcriptional regulator, PadR family | | CAETHG_1726 | CLJU_c38780 | CLRAG_21040 |
| 2650 | DNA-binding transcriptional regulator, PadR family | | CAETHG_3698 | CLJU_c16040 | CLRAG_33010 |
| 2651 | transcriptional regulator, RpiR family | | CAETHG_0221 | CLJU_c21350 | CLRAG_30910 |
| 2652 | transcriptional regulator, TetR family | | CAETHG_0459 | CLJU_c23930 | CLRAG_17230 |
| 2653 | transcriptional regulator, TetR family | | CAETHG_0631 | CLJU_c25620 | CLRAG_03820 |
| 2654 | transcriptional regulator, TetR family | | CAETHG_0936 | CLJU_c29420 | CLRAG_35410 |
| 2655 | transcriptional regulator, TetR family | | CAETHG_0940 | CLJU_c29460 | CLRAG_35450 |
| 2656 | transcriptional regulator, TetR family | | CAETHG_1388 | CLJU_c34900 | CLRAG_26140 |
| 2657 | transcriptional regulator, TetR family | | CAETHG_1493 | CLJU_c35860 | CLRAG_06360 |
| 2658 | transcriptional regulator, TetR family | | CAETHG_2189 | CLJU_c00720 | CLRAG_19860 |
| 2659 | transcriptional regulator, TetR family | | CAETHG_2470 | CLJU_c04070 | CLRAG_29180 |
| 2660 | transcriptional regulator, TetR family | | CAETHG_3141 | CLJU_c10510 | CLRAG_12830 |
| 2661 | transcriptional regulator, TetR family | | CAETHG_3491 | CLJU_c14100 | CLRAG_09450 |
| 2662 | transcriptional regulator, TraR/DksA family | | CAETHG_3908 | CLJU_c17990 | CLRAG_00640 |
| 2663 | transcriptional regulator, XRE family with cupin | | CAETHG_0377 | CLJU_c23140 | CLRAG_01670 |
| | sensor | | | | |
| 2664 | transcriptional regulator, XRE family with cupin | | CAETHG_3450 | CLJU_c13680 | CLRAG_10190 |
| | sensor | | | | |
| 2665 | glucokinase | 2.7.1.11 | CAETHG_0166 | CLJU_c20810 | CLRAG_19190 |
| 2666 | transketolase | 2.2.1.1 | CAETHG_2420 | CLJU_c03050 | CLRAG_28660 |
| 2667 | transketolase | 2.2.1.1 | CAETHG_2421 | CLJU_c03060 | CLRAG_28670 |
| 2668 | translation elongation factor 2 (EF-2/EF-G) | | CAETHG_1979 | CLJU_c41380 | CLRAG_23570 |
| 2669 | elongation factor G | | CAETHG_1950 | CLJU_c41070 | CLRAG_23260 |
| 2670 | bacterial translation initiation factor 1 (bIF-1) | | CAETHG_1925 | CLJU_c40820 | CLRAG_23010 |
| 2671 | transporter, NhaC family | | CAETHG_2983 | CLJU_c08880 | CLRAG_07600 |
| 2672 | Na+/H+ antiporter NhaD | | CAETHG_0728 | CLJU_c26470 | CLRAG_04560 |
| 2673 | trigger factor | | CAETHG_1473 | CLJU_c35650 | CLRAG_06180 |
| 2674 | triosephosphate isomerase | 5.3.1.1 | CAETHG_1758 | CLJU_c39130 | CLRAG_21280 |
| 2675 | triphosphoribosyl-dephospho-CoA synthase | | CAETHG_0606 | CLJU_c25370 | CLRAG_03640 |
| 2676 | tRNA-specific 2-thiouridylase | | CAETHG_0402 | CLJU_c23380 | CLRAG_01370 |
| 2677 | tRNA (guanine-N(7)-)-methyltransferase | | CAETHG_0964 | CLJU_c29660 | CLRAG_35660 |
| 2678 | tRNA (guanine37-N1)-methyltransferase | | CAETHG_3374 | CLJU_c12920 | CLRAG_10880 |
| 2679 | 23S rRNA (uracil-5-)-methyltransferase RumA | | CAETHG_2969 | CLJU_c08750 | CLRAG_07740 |
| 2680 | tRNA dimethylallyltransferase | | CAETHG_0208 | CLJU_c21220 | CLRAG_30800 |
| 2681 | poly(A) polymerase | | CAETHG_2258 | CLJU_c01520 | CLRAG_27180 |
| 2682 | tRNA pseudouridine38-40 synthase | 4.2.1.70 | CAETHG_1915 | CLJU_c40720 | CLRAG_22910 |
| 2683 | tRNA pseudouridine55 synthase | 4.2.1.70 | CAETHG_3401 | CLJU_c13180 | CLRAG_10610 |
| 2684 | tRNA-2-methylthio-N6-dimethylallyladenosine | | CAETHG_0211 | CLJU_c21250 | CLRAG_30830 |
| | synthase | | | | |
| 2685 | tRNA(adenine34) deaminase | | CAETHG_2192 | CLJU_c00760 | CLRAG_19820 |
| 2686 | tRNA-U20-dihydrouridine synthase | | CAETHG_1985 | CLJU_c41510 | CLRAG_04640 |
| 2687 | tRNA-U20a,U20b-dihydrouridine synthase | | CAETHG_1727 | CLJU_c38790 | CLRAG_21050 |
| 2688 | Trp operon repressor family | | CAETHG_1560 | CLJU_c36990 | CLRAG_36530 |
| 2689 | tryptophan synthase, alpha chain | | CAETHG_3707 | CLJU_c16130 | CLRAG_33100 |
| 2690 | tryptophan synthase beta chain | 4.2.1.20, | CAETHG_3706 | CLJU_c16120 | CLRAG_33090 |
| | | 4.1.2.8 | | | |
| 2691 | tryptophanyl-tRNA synthetase | | CAETHG_1686 | CLJU_c38290 | CLRAG_20760 |
| 2692 | TspO and MBR related proteins | | CAETHG_0611 | CLJU_c25420 | CLRAG_03700 |
| 2693 | HAMP domain-containing protein | | CAETHG_3269 | CLJU_c11780 | CLRAG_11760 |
| 2694 | two component transcriptional regulator, AraC | | CAETHG_1815 | CLJU_c39690 | CLRAG_21940 |
| | family | | | | |
| 2695 | two component transcriptional regulator, LuxR | | CAETHG_0883 | CLJU_c28870 | CLRAG_34950 |
| | family | | | | |
| 2696 | two component transcriptional regulator, LytTR | | CAETHG_1425 | CLJU_c35170 | CLRAG_05680 |
| | family | | | | |
| 2697 | two component transcriptional regulator, LytTR | | CAETHG_1588 | CLJU_c37320 | CLRAG_36760 |
| | family | | | | |
| 2698 | two component transcriptional regulator, LytTR | | CAETHG_3465 | CLJU_c13820 | CLRAG_10020 |
| | family | | | | |
| 2699 | DNA-binding response regulator, OmpR family, | | CAETHG_0041 | CLJU_c19640 | CLRAG_39500 |
| | contains REC and winged-helix (wHTH) domain | | | | |
| 2700 | DNA-binding response regulator, OmpR family, | | CAETHG_1195, | CLJU_c11770, | CLRAG_15360 |
| | contains REC and winged-helix (wHTH) domain | | CAETHG_3268 | CLJU_c38680, | |
| | | | | CLJU_c32970 | |
| 2701 | DNA-binding response regulator, OmpR family, | | CAETHG_3498, | CLJU_c14390, | CLRAG_09500 |
| | contains REC and winged-helix (wHTH) domain | | CAETHG_3513 | CLJU_c14170 | |
| 2702 | two-component system, NarL family, sensor | | CAETHG_0884 | CLJU_c28880 | CLRAG_34960 |
| | histidine kinase DegS | | | | |
| 2703 | leader peptidase (prepilin peptidase)/N- | | CAETHG_2643 | CLJU_c05610 | CLRAG_06740 |
| | methyltransferase | | | | |
| 2704 | type IV pilus assembly protein PilC | | CAETHG_3180 | CLJU_c10910 | CLRAG_12550 |
| 2705 | type III pantothenate kinase | 2.7.1.33 | CAETHG_1986 | CLJU_c41520 | CLRAG_04650 |
| 2706 | flagellum-specific ATP synthase | | CAETHG_3115 | CLJU_c10250 | CLRAG_13120 |
| 2707 | tyrosyl-tRNA synthetase | | CAETHG_1677 | CLJU_c38210 | CLRAG_20670 |
| 2708 | UDP-galactopyranose mutase | 5.4.99.9 | CAETHG_1252 | CLJU_c33530 | CLRAG_24750 |
| 2709 | UDP-glucose 4-epimerase | 5.1.3.7, | CAETHG_0258 | CLJU_c21710 | CLRAG_31300 |
| | | 5.1.3.2 | | | |
| 2710 | UDP-N-acetylglucosamine 1- | 2.5.1.7 | CAETHG_2027, | CLJU_c02460, | CLRAG_05090 |
| | carboxyvinyltransferase | | CAETHG_2352 | CLJU_c41980 | |
| 2711 | UDP-GlcNAc3NAcA epimerase | 5.1.3.14 | CAETHG_1305 | CLJU_c34070 | CLRAG_14220 |
| 2712 | UDP-N-acetylglucosamine 2-epimerase (non- | 5.1.3.14 | CAETHG_2341 | CLJU_c02350 | CLRAG_27960 |
| | hydrolysing) | | | | |
| 2713 | processive 1,2-diacylglycerol beta- | | CAETHG_2321 | CLJU_c02160 | CLRAG_27760 |
| | glucosyltransferase | | | | |
| 2714 | UDP-N-acetylglucosamine-N- | 2.4.1.227 | CAETHG_3028 | CLJU_c09330 | CLRAG_13770 |
| | acetylmuramylpentapeptide N-acetylglucosamine | | | | |
| | transferase | | | | |
| 2715 | UDP-N-acetylmuramate dehydrogenase | 1.1.1.158 | CAETHG_2433 | CLJU_c03180 | CLRAG_28790 |
| 2716 | UDP-N-acetylmuramate--L-alanine ligase | 6.3.2.8 | CAETHG_2010 | CLJU_c41810 | CLRAG_04940 |
| 2717 | UDP-N-acetylmuramoyl-tripeptide--D-alanyl-D- | 6.3.2.10, | CAETHG_3148 | CLJU_c10580 | CLRAG_12760 |
| | alanine ligase | 6.3.2.15 | | | |
| 2718 | UDP-N-acetylmuramoylalanine--D-glutamate | 6.3.2.9 | CAETHG_1980 | CLJU_c41450 | CLRAG_04580 |
| | ligase | | | | |
| 2719 | UDP-N-acetylmuramoylalanyl-D-glutamate--2,6- | 6.3.2.13 | CAETHG_3147 | CLJU_c10570 | CLRAG_12770 |
| | diaminopimelate ligase | | | | |
| 2720 | undecaprenyl diphosphate synthase | | CAETHG_1455 | CLJU_c35470 | CLRAG_06000 |
| 2721 | undecaprenyl diphosphate synthase | | CAETHG_3389 | CLJU_c13060 | CLRAG_10730 |
| 2722 | undecaprenyl-diphosphatase | 3.6.1.27 | CAETHG_2722 | CLJU_c06220 | CLRAG_07500 |
| 2723 | exopolysaccharide biosynthesis polyprenyl | | CAETHG_1300 | CLJU_c34020 | CLRAG_14170 |
| | glycosylphosphotransferase | | | | |
| 2724 | uracil phosphoribosyltransferase | 2.4.2.9 | CAETHG_2337 | CLJU_c02320 | CLRAG_27920 |
| 2725 | uridylate kinase | 2.7.4.14, | CAETHG_3387 | CLJU_c13040 | CLRAG_10750 |
| | | 2.7.4. -, | | | |
| | | 2.7.4.4, | | | |
| | | 2.7.4.22, | | | |
| | | 2.7.4.9 | | | |
| 2726 | urocanate hydratase | 4.2.1.49 | CAETHG_0234 | CLJU_c21480 | CLRAG_31040 |
| 2727 | uroporphyrinogen III methyltransferase/synthase | 2.1.1.107, | CAETHG_1125 | CLJU_c31970 | CLRAG_02600 |
| | | 4.2.1.75, | | | |
| | | 1.3.1.76 | | | |
| 2728 | UTP--glucose-1-phosphate uridylyltransferase | 2.7.7.9 | CAETHG_1318, | CLJU_c04510, | CLRAG_14340 |
| | | | CAETHG_2523 | CLJU_c34190 | |
| 2729 | valyl-tRNA synthetase | | CAETHG_1366 | CLJU_c34690 | CLRAG_14770 |
| 2730 | Glycopeptide antibiotics resistance protein | | CAETHG_3170 | CLJU_c10800 | CLRAG_22450 |
| 2731 | putative peptidoglycan lipid II flippase | | CAETHG_1302 | CLJU_c34040 | CLRAG_14190 |
| 2732 | Xaa-Pro dipeptidase | | CAETHG_0961 | CLJU_c29640 | CLRAG_35630 |
| 2733 | Xaa-Pro aminopeptidase | 3.4.11.1, | CAETHG_3189 | CLJU_c11000 | CLRAG_12460 |
| | | 3.4.11.2, | | | |
| | | 3.4.13.3, | | | |
| | | 3.4.11.23 | | | |
| 2734 | CO or xanthine dehydrogenase, Mo-binding | 1.1.1.204, | CAETHG_0993 | CLJU_c29940 | CLRAG_35940 |
| | subunit | 1.17.1.4 | | | |
| 2735 | xanthine dehydrogenase, molybdenum binding | 1.1.1.204, | CAETHG_0423 | CLJU_c23590 | CLRAG_17580 |
| | subunit apoprotein | 1.17.1.4 | | | |
| 2736 | CO or xanthine dehydrogenase, Mo-binding | | CAETHG_0456 | CLJU_c23900 | CLRAG_17260 |
| | subunit | | | | |
| 2737 | xylulokinase | 2.7.1.17 | CAETHG_3933 | CLJU_c18250 | CLRAG_00350 |
| 2738 | phospholipase C | | CAETHG_0300 | CLJU_c22020 | CLRAG_31550 |
| 2739 | Threonine dehydrogenase | 4.2.1.20, | CAETHG_0553 | CLJU_c24860 | CLRAG_17920 |
| | | 4.1.2.8 | | | |
| 2740 | Fe—S-cluster-containing dehydrogenase component | | CAETHG_0614 | CLJU_c25450 | * |
| 2741 | 1,4-dihydroxy-2-naphthoate prenyltransferase | 2.5.1.— | CAETHG_1874 | CLJU_c40280 | * |
| 2742 | 2-deoxy-D-gluconate 3-dehydrogenase | | CAETHG_0654 | CLJU_c25850 | * |
| 2743 | | | * | CLJU_c40790 | CLRAG_22980 |
| 2744 | 6-phosphogluconate dehydrogenase | 1.1.1.44 | CAETHG_3250 | CLJU_c11590 | * |
| 2745 | 6-pyruvoyltetrahydropterin/6- | | CAETHG_2465 | CLJU_c04020 | * |
| | carboxytetrahydropterin synthase | | | | |
| 2746 | ABC-2 type transport system ATP-binding protein | | CAETHG_0770 | CLJU_c26860 | * |
| 2747 | Abi-like protein | | CAETHG_1172 | CLJU_c32420 | * |
| 2748 | aconitase | 4.2.1.3, | CAETHG_1051, | CLJU_c30460, | * |
| | | 4.2.1.4 | CAETHG_2752 | CLJU_c06620 | |
| 2749 | Amino acid transporter | | CAETHG_2075 | CLJU_c42500 | * |
| 2750 | aldose 1-epimerase | 5.1.3.3 | CAETHG_3934 | CLJU_c18270 | * |
| 2751 | allantoinase | 3.5.2.5 | CAETHG_3635 | CLJU_c15330 | * |
| 2752 | putative amino-acid transport system ATP-binding | | CAETHG_0889 | CLJU_c27640, | * |
| | protein | | | CLJU_c28940 | |
| 2753 | putative amino-acid transport system substrate- | | CAETHG_0891 | CLJU_c28960 | * |
| | binding protein | | | | |
| 2754 | anaerobic sulfite reductase subunit A | 1.8.7.1 | CAETHG_0616 | CLJU_c25470 | * |
| 2755 | anaerobic sulfite reductase subunit B | 1.8.7.1 | CAETHG_0617 | CLJU_c25480 | * |
| 2756 | arginine deiminase | 3.5.3.6 | CAETHG_3021 | CLJU_c09270 | * |
| 2757 | arginine:ornithine antiporter/lysine permease | | CAETHG_3024 | CLJU_c09290, | * |
| | | | | CLJU_c27990 | |
| 2758 | aspartate carbamoyltransferase catalytic subunit | 2.1.3.2 | CAETHG_3631 | CLJU_c15290 | * |
| 2759 | branched-chain amino acid aminotransferase | 2.6.1.67, | CAETHG_2968 | CLJU_c08740 | CLRAG_07750 |
| | | 2.6.1.42, | | | |
| | | 2.6.1.6 | | | |
| 2760 | | | * | CLJU_c23570, | CLRAG_17600 |
| | | | | CLJU_c28000 | |
| 2761 | carbamate kinase | 2.7.2.2 | CAETHG_0421, | CLJU_c15300 | * |
| | | | CAETHG_3632 | | |
| 2762 | carbon storage regulator, CsrA | | CAETHG_3049 | CLJU_c09540 | * |
| 2763 | 4-carboxymuconolactone decarboxylase | 4.1.1.44 | CAETHG_1512 | CLJU_c36030 | * |
| 2764 | [citrate (pro-3S)-lyase] ligase | | CAETHG_0604 | CLJU_c25350 | * |
| 2765 | citrate lyase subunit gamma (acyl carrier protein) | 2.3.3.1 | CAETHG_0603, | CLJU_c30490, | * |
| | | | CAETHG_1054 | CLJU_c25340 | |
| 2766 | citrate lyase subunit alpha/citrate CoA- | 2.3.3.1 | CAETHG_0601, | CLJU_c30470, | * |
| | transferase | | CAETHG_1052 | CLJU_c25320 | |
| 2767 | citrate lyase subunit beta/citryl-CoA lyase | 2.3.3.1 | CAETHG_0602, | CLJU_c30480, | * |
| | | | CAETHG_1053 | CLJU_c25330 | |
| 2768 | D-3-phosphoglycerate dehydrogenase | 1.1.1.95 | CAETHG_3249 | CLJU_c11580 | * |
| 2769 | deoxyribonucleoside regulator | | CAETHG_3920 | CLJU_c18110 | * |
| 2770 | allantoinase | 3.5.2.5 | CAETHG_3636 | CLJU_c15340 | * |
| 2771 | electron transfer flavoprotein alpha subunit | | CAETHG_1868 | CLJU_c40220 | * |
| | apoprotein | | | | |
| 2772 | electron transfer flavoprotein beta subunit | | CAETHG_1869 | CLJU_c40230 | * |
| 2773 | 2,4-dienoyl-CoA reductase | | CAETHG_0869 | CLJU_c28740 | CLRAG_34820 |
| 2774 | 2,4-dienoyl-CoA reductase | | CAETHG_3711 | CLJU_c16160, | * |
| | | | | CLJU_c38590 | |
| 2775 | ethanolamine utilization protein EutN | | CAETHG_1824, | CLJU_c39780, | CLRAG_22030 |
| | | | CAETHG_3284 | CLJU_c11930 | |
| 2776 | nitrogen fixation protein NifB | | CAETHG_0418 | CLJU_c23540 | * |
| 2777 | Fur family transcriptional regulator, ferric uptake | | CAETHG_0018 | CLJU_c19410 | * |
| | regulator | | | | |
| 2778 | ferredoxin like protein | | CAETHG_1866 | CLJU_c40200 | * |
| 2779 | flagellin | | CAETHG_3058 | CLJU_c09630 | * |
| 2780 | diguanylate cyclase (GGDEF) domain-containing | | CAETHG_2464 | CLJU_c04010 | * |
| | protein | | | | |
| 2781 | gluconokinase | 2.7.1.12 | CAETHG_3252 | CLJU_c11610 | * |
| 2782 | Gnt-l system high-affinity gluconate transporter | | CAETHG_0816 | CLJU_c28160 | * |
| 2783 | | | * | CLJU_c28110, | CLRAG_09070 |
| | | | | CLJU_c38380 | |
| 2784 | glycerol 2-dehydrogenase (NAD+) | 1.1.1.6 | CAETHG_0738 | CLJU_c26570 | * |
| 2785 | glycerol dehydratase, cobalamin-independent, | 2.3.1.54 | CAETHG_3274 | CLJU_c11830 | * |
| | large subunit | | | | |
| 2786 | glycerol dehydratase, cobalamin-independent, | 2.3.1.54 | CAETHG_3275 | CLJU_c11840 | * |
| | small subunit | | | | |
| 2787 | putative glycosyltransferase, exosortase G- | | CAETHG_2463 | CLJU_c04000 | * |
| | associated | | | | |
| 2788 | Putative flippase GtrA (transmembrane | | CAETHG_1736 | CLJU_c38880 | * |
| | translocase of bactoprenol-linked glucose) | | | | |
| 2789 | LysR substrate binding domain-containing protein | | CAETHG_0002 | CLJU_c19260 | * |
| 2790 | similar to spore coat protein | | CAETHG_0029 | CLJU_c19520 | * |
| 2791 | vesicle tethering protein | | CAETHG_0039 | CLJU_c19620 | * |
| 2792 | hypothetical protein | | CAETHG_0082 | CLJU_c20020 | CLRAG_32500 |
| 2793 | Molybdopterin or thiamine biosynthesis | | CAETHG_0087, | CLJU_c20230, | CLRAG_29690 |
| | adenylyltransferase | | CAETHG_0104 | CLJU_c20060 | |
| 2794 | hypothetical protein | | CAETHG_0199 | CLJU_c21130 | * |
| 2795 | protein of unknown function (DUF4830) | | CAETHG_0202 | CLJU_c21160 | * |
| 2796 | hypothetical protein | | CAETHG_0203 | CLJU_c21170 | * |
| 2797 | hypothetical protein | | CAETHG_0212 | CLJU_c21260 | * |
| 2798 | hypothetical protein | | CAETHG_0214 | CLJU_c21280 | * |
| 2799 | Protein of unknown function (DUF1177) | | CAETHG_0281 | CLJU_c27890 | * |
| 2800 | hypothetical protein | | CAETHG_0295 | CLJU_c21970 | * |
| 2801 | protein of unknown function (DUF4430) | | CAETHG_0328 | CLJU_c22270, | CLRAG_31880 |
| | | | | CLJU_c22290 | |
| 2802 | Predicted TIM-barrel enzyme | | CAETHG_0362 | CLJU_c23000 | * |
| 2803 | GntR family transcriptional regulator/MocR | | CAETHG_0400 | CLJU_c23360 | * |
| | family aminotransferase | | | | |
| 2804 | GntR family transcriptional regulator/MocR | | CAETHG_0401 | CLJU_c23370 | * |
| | family aminotransferase | | | | |
| 2805 | hypothetical protein | 3.1.4.2, | CAETHG_0414 | CLJU_c23500 | * |
| | | 3.1.4.46 | | | |
| 2806 | hypothetical protein | | CAETHG_0479 | CLJU_c24210 | * |
| 2807 | protein of unknown function (DUF4111) | | CAETHG_0521 | CLJU_c24580 | * |
| 2808 | DNA polymerase beta domain protein region | | CAETHG_0522 | CLJU_c24590 | * |
| 2809 | GrpB domain, predicted nucleotidyltransferase, | | CAETHG_0526 | CLJU_c24620 | CLRAG_30000 |
| | UPF0157 family | | | | |
| 2810 | hypothetical protein | | CAETHG_0623 | CLJU_c30950, | * |
| | | | | CLJU_c27570, | |
| | | | | CLJU_c25540 | |
| 2811 | SdpI/YhfL protein family protein | | CAETHG_0627, | CLJU_c25580, | * |
| | | | CAETHG_3804 | CLJU_c16940 | |
| 2812 | Quinol monooxygenase YgiN | | CAETHG_0629 | CLJU_c25600 | * |
| 2813 | CDP-diacylglycerol--glycerol-3-phosphate 3- | | CAETHG_0642 | CLJU_c25730 | CLRAG_03930 |
| | phosphatidyltransferase | | | | |
| 2814 | hypothetical protein | | CAETHG_0668 | CLJU_c25990 | * |
| 2815 | Uncharacterized conserved protein (DUF2149) | | CAETHG_0669 | CLJU_c26000 | * |
| 2816 | Biopolymer transport protein ExbB/TolQ | | CAETHG_0670 | CLJU_c26010 | * |
| 2817 | Fibronectin type III domain-containing protein | | CAETHG_0672 | CLJU_c26030 | * |
| 2818 | hypothetical protein | | CAETHG_0679 | CLJU_c26100 | * |
| 2819 | Type I phosphodiesterase/nucleotide | | CAETHG_0711 | CLJU_c26310 | * |
| | pyrophosphatase | | | | |
| 2820 | hypothetical protein | | CAETHG_0722 | CLJU_c26410 | * |
| 2821 | ABC-2 family transporter protein | | CAETHG_0769 | CLJU_c26850 | * |
| 2822 | Uncharacterized membrane protein | | CAETHG_0772 | CLJU_c26880 | * |
| 2823 | protein of unknown function (DUF2935) | | CAETHG_0782 | CLJU_c26980 | * |
| 2824 | hypothetical protein | | CAETHG_0783 | CLJU_c26990 | * |
| 2825 | hypothetical protein | | CAETHG_0807 | CLJU_c27220 | * |
| 2826 | Acetyl esterase/lipase | | CAETHG_0864 | CLJU_c28690 | CLRAG_34770 |
| 2827 | hypothetical protein | | CAETHG_0935 | CLJU_c29410 | * |
| 2828 | Protein of unknown function DUF2680 | | CAETHG_0955 | CLJU_c29590 | CLRAG_35580 |
| 2829 | Short C-terminal domain-containing protein | | CAETHG_0958 | CLJU_c29620 | CLRAG_35610 |
| 2830 | 4Fe—4S binding domain-containing protein | | CAETHG_0959 | CLJU_c29630 | CLRAG_35620 |
| 2831 | uroporphyrinogen decarboxylase | | CAETHG_1046 | CLJU_c30410 | * |
| 2832 | Haem-degrading | | CAETHG_1071 | CLJU_c30670 | * |
| 2833 | hypothetical protein | | CAETHG_1089 | CLJU_c30890 | CLRAG_16270 |
| 2834 | protein of unknown function (DUF4829) | | CAETHG_1090 | CLJU_c30900 | CLRAG_16280 |
| 2835 | hypothetical protein | | CAETHG_1092 | CLJU_c30920 | CLRAG_16300 |
| 2836 | protein of unknown function (DUF4829) | | CAETHG_1098 | CLJU_c30930 | CLRAG_16310 |
| 2837 | hypothetical protein | | CAETHG_1103 | CLJU_c31050 | * |
| 2838 | hypothetical protein | | CAETHG_1155 | CLJU_c32270 | CLRAG_23940 |
| 2839 | TfoX N-terminal domain-containing protein | | CAETHG_1161 | CLJU_c32310 | CLRAG_08870 |
| 2840 | hypothetical protein | | CAETHG_1165, | CLJU_c32350 | CLRAG_37640 |
| | | | CAETHG_1658 | | |
| 2841 | Abi-like protein | | CAETHG_1171 | CLJU_c32410 | * |
| 2842 | epoxyqueuosine reductase | | CAETHG_1173 | CLJU_c32750 | * |
| 2843 | hypothetical protein | | CAETHG_1178 | CLJU_c32800 | * |
| 2844 | hypothetical protein | | CAETHG_1326 | CLJU_c34270 | * |
| 2845 | hypothetical protein | | CAETHG_1327 | CLJU_c34280 | * |
| 2846 | hypothetical protein | | CAETHG_1379 | CLJU_c34810 | * |
| 2847 | hypothetical protein | | CAETHG_1391 | CLJU_c34930 | CLRAG_26170 |
| 2848 | Protein of unknown function (DUF1648) | | CAETHG_1410 | CLJU_c35010 | * |
| 2849 | DNA transformation protein | | CAETHG_1419 | CLJU_c35100 | * |
| 2850 | Zn-dependent protease (includes SpoIVFB) | | CAETHG_1422 | CLJU_c35140 | CLRAG_26390 |
| 2851 | hypothetical protein | | CAETHG_1439 | CLJU_c35300 | * |
| 2852 | Protein of unknown function (DUF3892) | | CAETHG_1523 | CLJU_c36130 | * |
| 2853 | hypothetical protein | | CAETHG_1534 | CLJU_c36240 | * |
| 2854 | hypothetical protein | | CAETHG_1541 | CLJU_c36330 | * |
| 2855 | hypothetical protein | | CAETHG_1542 | CLJU_c36340 | * |
| 2856 | hypothetical protein | | CAETHG_1544 | CLJU_c36360 | * |
| 2857 | protein of unknown function (DUF4878) | | CAETHG_1708 | CLJU_c38550 | * |
| 2858 | Protein of unknown function (DUF2889) | | CAETHG_1725 | CLJU_c38770 | * |
| 2859 | hypothetical protein | | CAETHG_1739 | CLJU_c38910 | * |
| 2860 | hypothetical protein | | CAETHG_1768 | CLJU_c39230 | * |
| 2861 | Dolichyl-phosphate-mannose-protein | | CAETHG_1848 | CLJU_c40010 | * |
| | mannosyltransferase | | | | |
| 2862 | protein of unknown function (DUF2935) | | CAETHG_1851 | CLJU_c40040 | * |
| 2863 | hypothetical protein | | CAETHG_1857 | CLJU_c40080 | * |
| 2864 | Calcineurin-like phosphoesterase | | CAETHG_1865 | CLJU_c40180 | * |
| 2865 | YtkA-like | | CAETHG_1878 | CLJU_c40320 | * |
| 2866 | hypothetical protein | | CAETHG_2080 | CLJU_c42540 | * |
| 2867 | hypothetical protein | | CAETHG_2142 | CLJU_c00250 | * |
| 2868 | AAA-like domain-containing protein | | CAETHG_2161 | CLJU_c00430 | * |
| 2869 | hypothetical protein | | CAETHG_2162 | CLJU_c00440 | * |
| 2870 | hypothetical protein | | CAETHG_2163 | CLJU_c00450 | * |
| 2871 | hypothetical protein | | CAETHG_2165 | CLJU_c00460 | * |
| 2872 | DNA-binding transcriptional regulator, XRE-family | | CAETHG_2166 | CLJU_c00470 | CLRAG_33390 |
| | HTH domain | | | | |
| 2873 | Major Facilitator Superfamily protein | | CAETHG_2450 | CLJU_c03880 | * |
| 2874 | hypothetical protein | | CAETHG_2452 | CLJU_c03900 | * |
| 2875 | succinyl-diaminopimelate desuccinylase | | CAETHG_2453 | CLJU_c03910 | * |
| 2876 | Firmicu-CTERM domain-containing protein | | CAETHG_2461 | CLJU_c03980 | * |
| 2877 | exosortase family protein XrtG | | CAETHG_2462 | CLJU_c03990 | * |
| 2878 | hypothetical protein | | CAETHG_2466 | CLJU_c04030 | * |
| 2879 | conserved repeat domain-containing protein | | CAETHG_2495 | CLJU_c04270 | * |
| 2880 | hypothetical protein | | CAETHG_2496 | CLJU_c04280 | * |
| 2881 | ABC-2 type transport system permease protein | | CAETHG_2580 | CLJU_c05030 | CLRAG_38420 |
| 2882 | hypothetical protein | | CAETHG_2582 | CLJU_c05050 | CLRAG_38440 |
| 2883 | hypothetical protein | | CAETHG_2669 | CLJU_c05750 | * |
| 2884 | hypothetical protein | | CAETHG_2671 | CLJU_c05770 | * |
| 2885 | Protein of unknown function (DUF2442) | | CAETHG_2685, | CLJU_c13720 | * |
| | | | CAETHG_3454 | | |
| 2886 | hypothetical protein | | CAETHG_2694 | CLJU_c05970 | * |
| 2887 | hypothetical protein | | CAETHG_2734 | CLJU_c06390 | * |
| 2888 | VWA domain containing CoxE-like protein | | CAETHG_2738 | CLJU_c06420 | * |
| 2889 | hypothetical protein | | CAETHG_2739 | CLJU_c06430 | * |
| 2890 | hypothetical protein | | CAETHG_2740 | CLJU_c06440 | * |
| 2891 | hypothetical protein | | CAETHG_2859 | CLJU_c07660 | * |
| 2892 | hypothetical protein | | CAETHG_2860 | CLJU_c07670 | * |
| 2893 | ABC-2 type transport system permease protein | | CAETHG_2869 | CLJU_c07770 | * |
| 2894 | 2-enoate reductase | | CAETHG_2913 | CLJU_c08180 | * |
| 2895 | hypothetical protein | | CAETHG_2924 | CLJU_c08290 | * |
| 2896 | PD-(D/E)XK nuclease superfamily | | CAETHG_2925 | CLJU_c08300 | * |
| 2897 | hypothetical protein | | CAETHG_2929 | CLJU_c08340 | * |
| 2898 | hypothetical protein | | CAETHG_2930 | CLJU_c08350 | * |
| 2899 | hypothetical protein | | CAETHG_2931 | CLJU_c08370 | * |
| 2900 | Methyltransferase domain-containing protein | | CAETHG_3060 | CLJU_c09650 | * |
| 2901 | Protein of unknown function (DUF2920) | | CAETHG_3061 | CLJU_c09660 | * |
| 2902 | lysine-N-methylase | | CAETHG_3062 | CLJU_c09670 | * |
| 2903 | hypothetical protein | | CAETHG_3063 | CLJU_c09680 | * |
| 2904 | Predicted AAA-ATPase | | CAETHG_3086 | CLJU_c09950, | CLRAG_13410 |
| | | | | CLJU_c09890 | |
| 2905 | Predicted nucleotidyltransferase | | CAETHG_3087 | CLJU_c09960 | * |
| 2906 | Uncharacterized conserved protein YutE, | | CAETHG_3088 | CLJU_c09970 | * |
| | UPF0331/DUF86 family | | | | |
| 2907 | hypothetical protein | | CAETHG_3436 | CLJU_c13520 | * |
| 2908 | Flavodoxin | | CAETHG_3504 | CLJU_c14220 | * |
| 2909 | purine catabolism regulatory protein | | CAETHG_3627 | CLJU_c15250 | * |
| 2910 | (S)-ureidoglycine aminohydrolase | | CAETHG_3629 | CLJU_c15270 | * |
| 2911 | HlyD family secretion protein | | CAETHG_3649 | CLJU_c15480 | * |
| 2912 | hypothetical protein | | CAETHG_3716 | CLJU_c16220 | CLRAG_33130 |
| 2913 | hypothetical protein | | CAETHG_3717 | CLJU_c16230 | CLRAG_33140 |
| 2914 | hypothetical protein | | CAETHG_3729 | CLJU_c16350 | CLRAG_33160 |
| 2915 | hypothetical protein | | CAETHG_3739, | CLJU_c16450, | * |
| | | | CAETHG_3742 | CLJU_c16470 | |
| 2916 | hypothetical protein | | CAETHG_3746 | CLJU_c16500 | * |
| 2917 | hypothetical protein | | CAETHG_3763 | CLJU_c16590 | CLRAG_33490 |
| 2918 | hypothetical protein | | CAETHG_3805 | CLJU_c16950 | * |
| 2919 | protein of unknown function (DUF3784) | | CAETHG_3806 | CLJU_c16960 | * |
| 2920 | hypothetical protein | | CAETHG_3808 | CLJU_c16980 | * |
| 2921 | hypothetical protein | | CAETHG_3816 | CLJU_c17040 | * |
| 2922 | Uncharacterized membrane protein YcaP, DUF421 | | CAETHG_3817 | CLJU_c17050 | * |
| | family | | | | |
| 2923 | Uncharacterized SAM-binding protein YcdF, | | CAETHG_3854 | CLJU_c17410 | * |
| | DUF218 family | | | | |
| 2924 | Protein-S-isoprenylcysteine O-methyltransferase | | CAETHG_3856 | CLJU_c17430 | * |
| | Stel4 | | | | |
| 2925 | chromate transporter | | CAETHG_3866 | CLJU_c17580 | * |
| 2926 | L-2-amino-thiazoline-4-carboxylic acid hydrolase | | CAETHG_3876 | CLJU_c17680 | CLRAG_01060 |
| 2927 | HEPN domain-containing protein | | CAETHG_3937 | CLJU_c18300 | * |
| 2928 | Arylsulfotransferase (ASST) | | CAETHG_3941 | CLJU_c18340 | * |
| 2929 | hypothetical protein | | CAETHG_3945 | CLJU_c18380 | * |
| 2930 | hypothetical protein | | CAETHG_3960 | CLJU_c18530 | * |
| 2931 | hypothetical protein | | CAETHG_3961 | CLJU_c18540 | * |
| 2932 | virus Gpl57 | | CAETHG_3962 | CLJU_c18550 | * |
| 2933 | hypothetical protein | | CAETHG_3963 | CLJU_c18560 | * |
| 2934 | phage-like protein | | CAETHG_3979 | CLJU_c18650 | * |
| 2935 | Methyltransferase domain-containing protein | | CAETHG_4031 | CLJU_c18970 | * |
| 2936 | G5 domain-containing protein | | CAETHG_4035 | CLJU_c19010 | * |
| 2937 | hypothetical protein | | CAETHG_4040 | CLJU_c19060 | * |
| 2938 | Cys/Met metabolism PLP-dependent enzyme | 2.5.1.—, | CAETHG_4050 | CLJU_c19160 | * |
| | | 2.5.1.48, | | | |
| | | 2.5.1.49, | | | |
| | | 4.2.99.9 | | | |
| 2939 | | | * | CLJU_c27480 | CLRAG_08940 |
| 2940 | | | * | CLJU_c25630 | CLRAG_03830 |
| 2941 | | | * | CLJU_c36220, | CLRAG_23960 |
| | | | | CLJU_c36370 | |
| 2942 | | | * | CLJU_c31600 | CLRAG_32430 |
| 2943 | | | * | CLJU_c00980 | CLRAG_19600 |
| 2944 | | | * | CLJU_c31810 | CLRAG_02440 |
| 2945 | | | * | CLJU_c14450 | CLRAG_09750 |
| 2946 | | | * | CLJU_c30730 | CLRAG_16170 |
| 2947 | | | * | CLJU_c07810, | CLRAG_16800 |
| | | | | CLJU_c03710 | |
| 2948 | | | * | CLJU_c31540 | CLRAG_16380 |
| 2949 | | | * | CLJU_c42750 | CLRAG_25560 |
| 2950 | | | * | CLJU_c30820 | CLRAG_16260 |
| 2951 | | | * | CLJU_c22510 | CLRAG_32090 |
| 2952 | Nicotinamidase-related amidase | | CAETHG_2912 | CLJU_c08170 | * |
| 2953 | ketol-acid reductoisomerase | 1.1.1.86, | CAETHG_0122 | CLJU_c20400, | CLRAG_25900 |
| | | 1.1.1.169, | | CLJU_c20390 | |
| | | 5.4.99.3 | | | |
| 2954 | ketol-acid reductoisomerase | 1.1.1.86, | CAETHG_3633 | CLJU_c15310 | * |
| | | 1.1.1.169, | | | |
| | | 5.4.99.3 | | | |
| 2955 | L-rhamnose isomerase | 5.3.1.14 | CAETHG_2086 | CLJU_c42600 | * |
| 2956 | rhamnulokinase | 2.7.1.5 | CAETHG_2087 | CLJU_c42610 | * |
| 2957 | | | * | CLJU_c26820 | CLRAG_08700 |
| 2958 | 5-methyltetrahydrofolate--homocysteine | 2.1.1.13, | CAETHG_0145, | CLJU_c20690, | CLRAG_19360 |
| | methyltransferase | 2.1.1.14 | CAETHG_0153 | CLJU_c20620 | |
| 2959 | methyl-accepting chemotaxis sensory transducer | | CAETHG_2073 | CLJU_c42480 | * |
| | with Cache sensor | | | | |
| 2960 | methyl-accepting chemotaxis sensory transducer | | CAETHG_3026 | CLJU_c09310 | * |
| | with Cache sensor | | | | |
| 2961 | methyl-accepting chemotaxis sensory transducer | | CAETHG_3492 | CLJU_c14110 | * |
| | with Cache sensor | | | | |
| 2962 | | | * | CLJU_c29230 | CLRAG_35230 |
| 2963 | methyl-accepting chemotaxis protein | | CAETHG_1214 | CLJU_c33150 | CLRAG_15140 |
| 2964 | methyl-accepting chemotaxis sensory transducer | | CAETHG_3981 | CLJU_c18670 | * |
| | with TarH sensor/methyl-accepting chemotaxis | | | | |
| | sensory transducer with Cache sensor | | | | |
| 2965 | molybdopterin molybdochelatase | | CAETHG_0098 | CLJU_c20170 | CLRAG_29750 |
| 2966 | molybdopterin molybdochelatase | | CAETHG_0099 | CLJU_c20180 | CLRAG_29740 |
| 2967 | N-acyl-D-amino-acid deacylase | | CAETHG_0995 | CLJU_c29960 | CLRAG_35960 |
| 2968 | allantoate deiminase | | CAETHG_3630 | CLJU_c15280 | * |
| 2969 | malate dehydrogenase (oxaloacetate- | | CAETHG_0605, | CLJU_c30500, | * |
| | decarboxylating) | | CAETHG_1055 | CLJU_c25360 | |
| 2970 | sulfonate transport system substrate-binding | | CAETHG_3940 | CLJU_c18330 | * |
| | protein | | | | |
| 2971 | nitrogenase molybdenum-iron protein alpha chain | | CAETHG_0416 | CLJU_c23520 | * |
| 2972 | nitrogenase molybdenum-iron protein beta chain | | CAETHG_0415 | CLJU_c23510 | * |
| 2973 | non-heme chloroperoxidase | | CAETHG_1085 | CLJU_c30810 | * |
| 2974 | ornithine carbamoyltransferase | 2.1.3.3 | CAETHG_3022 | CLJU_c28010, | * |
| | | | | CLJU_c09280 | |
| 2975 | Site-specific recombinase XerD | | CAETHG_2138 | CLJU_c00210 | * |
| 2976 | Helix-turn-helix domain-containing protein | | CAETHG_3751 | CLJU_c16550 | CLRAG_33360 |
| 2977 | Phosphoglycerate dehydrogenase | 1.1.1.95 | CAETHG_1012 | CLJU_c30120 | * |
| 2978 | prephenate dehydratase | 4.2.1.91, | CAETHG_0619 | CLJU_c25500 | * |
| | | 4.2.1.51 | | | |
| 2979 | Propanediol utilization protein | | CAETHG_1818, | CLJU_c39720, | CLRAG_21970 |
| | | | CAETHG_3288 | CLJU_c11970 | |
| 2980 | iron complex transport system substrate-binding | | CAETHG_3824, | CLJU_c17120, | * |
| | protein | | CAETHG_3830 | CLJU_c17180 | |
| 2981 | tungstate transport system ATP-binding protein | | CAETHG_0097 | CLJU_c20160 | CLRAG_29760 |
| 2982 | putative spermidine/putrescine transport system | | CAETHG_0707 | CLJU_c26270 | * |
| | ATP-binding protein | | | | |
| 2983 | ABC-2 type transport system ATP-binding protein | | CAETHG_2581 | CLJU_c05040 | CLRAG_38430 |
| 2984 | ABC-type lipoprotein export system, ATPase | | CAETHG_3835 | CLJU_c17220 | * |
| | component | | | | |
| 2985 | tungstate transport system substrate-binding | | CAETHG_0095 | CLJU_c20140 | CLRAG_29780 |
| | protein | | | | |
| 2986 | D-methionine transport system substrate-binding | | CAETHG_2726 | CLJU_c06300 | * |
| | protein | | | | |
| 2987 | tungstate transport system permease protein | | CAETHG_0096 | CLJU_c20150 | CLRAG_29770 |
| 2988 | putative spermidine/putrescine transport system | | CAETHG_0708 | CLJU_c26280 | * |
| | permease protein | | | | |
| 2989 | putative spermidine/putrescine transport system | | CAETHG_0709 | CLJU_c26290 | * |
| | permease protein | | | | |
| 2990 | D-methionine transport system permease protein | | CAETHG_2725 | CLJU_c06290 | * |
| 2991 | putative ABC transport system permease protein | | CAETHG_3651 | CLJU_c15500 | * |
| 2992 | ElaA protein | | CAETHG_0806 | CLJU_c27210 | * |
| 2993 | Ribosomal protein S18 acetylase RimI | | CAETHG_1417 | CLJU_c35080 | CLRAG_26340 |
| 2994 | Acetyltransferase (GNAT) domain-containing | | CAETHG_1420 | CLJU_c35110 | * |
| | protein | | | | |
| 2995 | | | * | CLJU_c21000 | CLRAG_19020 |
| 2996 | Acyl-CoA thioester hydrolase/BAAT N-terminal | 3.1.2. | CAETHG_0718 | CLJU_c26370 | CLRAG_04450 |
| | region | | | | |
| 2997 | Imidazolonepropionase | 3.5.2.3 | CAETHG_1002 | CLJU_c30030 | * |
| 2998 | hypothetical protein | | CAETHG_2079 | CLJU_c42530 | * |
| 2999 | amidohydrolase | 3.5.1.47 | CAETHG_2723 | CLJU_c06270 | * |
| 3000 | putative amino-acid transport system permease | | CAETHG_0890 | CLJU_c28950 | * |
| | protein | | | | |
| 3001 | N-acyl-D-amino-acid deacylase | | CAETHG_0259 | CLJU_c21720 | * |
| 3002 | Peptidase family M28 | | CAETHG_1859 | CLJU_c40100 | * |
| 3003 | Sugar phosphate isomerase/epimerase | | CAETHG_0792 | CLJU_c27080 | * |
| 3004 | holo-ACP synthase | | CAETHG_0598 | CLJU_c25290 | * |
| 3005 | AraC-like ligand binding domain-containing protein | | CAETHG_3438 | CLJU_c13540 | * |
| 3006 | AAA domain (dynein-related subfamily) | | CAETHG_2733 | CLJU_c06380 | * |
| 3007 | tripartite ATP-independent transporter solute | | CAETHG_3255 | CLJU_c11640 | * |
| | receptor, DctP family | | | | |
| 3008 | Putative cell wall binding repeat 2 | | CAETHG_0327, | CLJU_c22350, | CLRAG_31870 |
| | | | CAETHG_0335 | CLJU_c22280 | |
| 3009 | protein of unknown function (DUF4430) | | CAETHG_0333 | CLJU_c22330 | CLRAG_31920 |
| 3010 | Putative cell wall-binding protein | | CAETHG_1424 | CLJU_c35160 | * |
| 3011 | Putative cell wall binding repeat 2 | | CAETHG_2687 | CLJU_c05900 | * |
| 3012 | Putative cell wall binding repeat 2 | | CAETHG_3812 | CLJU_c17000 | * |
| 3013 | methyl-accepting chemotaxis protein | | CAETHG_1045 | CLJU_c30400 | * |
| 3014 | Methyl-accepting chemotaxis protein (MCP) | | CAETHG_3863 | CLJU_c17500 | * |
| | signalling domain-containing protein | | | | |
| 3015 | chromate transporter | | CAETHG_1527 | CLJU_c36170 | CLRAG_24010 |
| 3016 | malate:Na+ symporter | 2.3.3.1 | CAETHG_1701, | CLJU_c38450 | * |
| | | | CAETHG_2480 | | |
| 3017 | Methanogenic corrinoid protein MtbC1 | | CAETHG_2844, | CLJU_c07560, | * |
| | | | CAETHG_2849 | CLJU_c07510 | |
| 3018 | GTPase, G3E family | | CAETHG_4042 | CLJU_c19080 | * |
| 3019 | energy-coupling factor transport system ATP- | | CAETHG_0330 | CLJU_c22310 | CLRAG_31900 |
| | binding protein | | | | |
| 3020 | energy-coupling factor transport system permease | | CAETHG_0332 | CLJU_c22320 | CLRAG_31910 |
| | protein | | | | |
| 3021 | nucleoside transport protein | | CAETHG_3923 | CLJU_c18140 | * |
| 3022 | D-3-phosphoglycerate dehydrogenase | 1.1.1.95 | CAETHG_2448 | CLJU_c03860 | * |
| 3023 | D-3-phosphoglycerate dehydrogenase | 1.1.1.95 | CAETHG_3253 | CLJU_c11620 | * |
| 3024 | putative selenium metabolism hydrolase | 3.5.1.14, | CAETHG_0994 | CLJU_c29950 | CLRAG_35950 |
| | | 3.5.1.16 | | | |
| 3025 | death on curing protein | | CAETHG_1097 | CLJU_c32640 | CLRAG_29380 |
| 3026 | MutS domain V | | CAETHG_0794 | CLJU_c27090 | * |
| 3027 | bla regulator protein blaR1 | | CAETHG_2770 | CLJU_c06790 | CLRAG_18590 |
| 3028 | Acetyl esterase/lipase | | CAETHG_0863 | CLJU_c28680 | CLRAG_34760 |
| 3029 | hypothetical protein | | CAETHG_1331 | CLJU_c34310 | * |
| 3030 | Enterochelin esterase | | CAETHG_1418 | CLJU_c35090 | CLRAG_26350 |
| 3031 | | | * | CLJU_c37370 | CLRAG_36810 |
| 3032 | | | * | CLJU_c29650 | CLRAG_35640 |
| 3033 | Ethanolamine utilization cobalamin | | CAETHG_1827, | CLJU_c39810, | CLRAG_22060 |
| | adenosyltransferase | | CAETHG_3281 | CLJU_c11900 | |
| 3034 | putative spermidine/putrescine transport system | | CAETHG_0710 | CLJU_c26300 | * |
| | substrate-binding protein | | | | |
| 3035 | electron transfer flavoprotein-quinone | | CAETHG_1867 | CLJU_c40210 | * |
| | oxidoreductase | | | | |
| 3036 | | | * | CLJU_c00150 | CLRAG_20170 |
| 3037 | hypothetical protein | | CAETHG_0780 | CLJU_c26960 | * |
| 3038 | radical SAM additional 4Fe4S-binding SPASM | | CAETHG_1535 | CLJU_c36250 | * |
| | domain-containing protein | | | | |
| 3039 | ABC-type Fe3+ transport system, substrate-binding | | CAETHG_3829 | CLJU_c17170 | * |
| | protein | | | | |
| 3040 | Uncharacterized 2Fe-2 and 4Fe—4S clusters- | | CAETHG_4047 | CLJU_c19130 | * |
| | containing protein, contains DUF4445 domain | | | | |
| 3041 | flagellar hook-associated protein 3 FlgL | | CAETHG_3047 | CLJU_c09520 | * |
| 3042 | chemotaxis protein MotA | | CAETHG_0049 | CLJU_c19720 | CLRAG_39420 |
| 3043 | chemotaxis protein MotB | | CAETHG_0048 | CLJU_c19710 | CLRAG_39430 |
| 3044 | | | * | CLJU_c09550 | CLRAG_13560 |
| 3045 | | | * | CLJU_c37490 | CLRAG_36900 |
| 3046 | Ribosomal protein S18 acetylase RimI | | CAETHG_2992 | CLJU_c08980 | * |
| 3047 | diguanylate cyclase (GGDEF) domain-containing | | CAETHG_0173 | CLJU_c20880 | * |
| | protein | | | | |
| 3048 | | | * | CLJU_c28080 | CLRAG_08980 |
| 3049 | Glycosyltransferase involved in cell wall | | CAETHG_2774 | CLJU_c06830 | * |
| | bisynthesis | | | | |
| 3050 | Dolichyl-phosphate-mannose-protein | | CAETHG_1738 | CLJU_c38900 | * |
| | mannosyltransferase | | | | |
| 3051 | dolichol-phosphate mannosyltransferase | | CAETHG_2458 | CLJU_c03960 | * |
| 3052 | Tetratricopeptide repeat-containing protein | | CAETHG_3059 | CLJU_c09640 | * |
| 3053 | hypothetical protein | | CAETHG_3085 | CLJU_c09940 | * |
| 3054 | Glycosyltransferase involved in cell wall | | CAETHG_4033 | CLJU_c18990 | * |
| | bisynthesis | | | | |
| 3055 | hypothetical protein | | CAETHG_0379 | CLJU_c23160 | * |
| 3056 | Putative flippase GtrA (transmembrane | | CAETHG_2067 | CLJU_c42420 | * |
| | translocase of bactoprenol-linked glucose) | | | | |
| 3057 | Sulfite exporter TauE/SafE | | CAETHG_1870 | CLJU_c40240 | * |
| 3058 | putative ATPase | | CAETHG_3291 | CLJU_c12000 | * |
| 3059 | Phage integrase family protein | | CAETHG_0767, | CLJU_c14790, | CLRAG_08710 |
| | | | CAETHG_3579 | CLJU_c26830 | |
| 3060 | iron complex transport system ATP-binding | | CAETHG_0088 | CLJU_c20070 | CLRAG_29590 |
| | protein | | | | |
| 3061 | iron complex transport system permease protein | | CAETHG_0089 | CLJU_c20080 | CLRAG_29600 |
| 3062 | Polyferredoxin | | CAETHG_1879 | CLJU_c40330 | * |
| 3063 | iron complex transport system substrate-binding | | CAETHG_0090 | CLJU_c20090 | CLRAG_29660 |
| | protein | | | | |
| 3064 | Protein-S-isoprenylcysteine O-methyltransferase | | CAETHG_2175 | CLJU_c00570 | * |
| | Ste14 | | | | |
| 3065 | L-rhamnose mutarotase | | CAETHG_2083 | CLJU_c42570 | * |
| 3066 | DNA-binding transcriptional regulator, LysR family | | CAETHG_3712 | CLJU_c16170 | * |
| 3067 | hypothetical protein | | CAETHG_0517 | CLJU_c24550 | * |
| 3068 | Transmembrane secretion effector | | CAETHG_0518 | CLJU_c24560 | * |
| 3069 | glycerol uptake facilitator protein | | CAETHG_3280 | CLJU_c11890 | * |
| 3070 | DNA-binding transcriptional regulator, MarR family | | CAETHG_1175 | CLJU_c32770 | * |
| 3071 | DNA-binding transcriptional regulator, MarR family | | CAETHG_1209 | CLJU_c33110 | CLRAG_15220 |
| 3072 | DNA-binding transcriptional regulator, MarR family | | CAETHG_3855 | CLJU_c17420 | * |
| 3073 | Zn-dependent protease (includes SpoIVFB) | | CAETHG_0038 | CLJU_c19610 | * |
| 3074 | energy-coupling factor transport system substrate- | | CAETHG_0329 | CLJU_c22300 | CLRAG_31890 |
| | specific component | | | | |
| 3075 | Calcineurin-like phosphoesterase | | CAETHG_3505 | CLJU_c14230 | * |
| 3076 | methyl-accepting chemotaxis protein | | CAETHG_3913 | CLJU_c18040 | * |
| 3077 | Ubiquinone/menaquinone biosynthesis C- | | CAETHG_0499 | CLJU_c24390 | * |
| | methylase UbiE | | | | |
| 3078 | | | * | CLJU_c22620 | CLRAG_32180 |
| 3079 | | | * | CLJU_c24100 | CLRAG_30580 |
| 3080 | Sugar phosphate permease | | CAETHG_0865 | CLJU_c28700 | CLRAG_34780 |
| 3081 | Predicted arabinose efflux permease, MFS family | | CAETHG_0866 | CLJU_c28710 | CLRAG_34790 |
| 3082 | magnesium transporter | | CAETHG_3007 | CLJU_c09130 | * |
| 3083 | Sulfur carrier protein ThiS (thiamine biosynthesis) | | CAETHG_0086, | CLJU_c20220, | CLRAG_34300 |
| | | | CAETHG_0103 | CLJU_c20050 | |
| 3084 | molybdate transport system substrate-binding | | CAETHG_0671 | CLJU_c26020 | * |
| | protein | | | | |
| 3085 | Molybdopterin oxidoreductase Fe4S4 domain- | 1.7.7.2 | CAETHG_0613 | CLJU_c25440 | * |
| | containing protein | | | | |
| 3086 | molybdenum-pterin binding domain-containing | | CAETHG_0001 | CLJU_c19250 | * |
| | protein | | | | |
| 3087 | | | * | CLJU_c19740 | CLRAG_39400 |
| 3088 | putative efflux protein, MATE family | | CAETHG_1208 | CLJU_c33100 | CLRAG_15230 |
| 3089 | putative efflux protein, MATE family | | CAETHG_0796 | CLJU_c27110 | * |
| 3090 | | | * | CLJU_c22520 | CLRAG_32100 |
| 3091 | | | * | CLJU_c22710 | CLRAG_02070 |
| 3092 | 2,4-dienoyl-CoA reductase | | CAETHG_0867 | CLJU_c28720 | CLRAG_34800 |
| 3093 | Nitroreductase | | CAETHG_3628 | CLJU_c15260 | * |
| 3094 | Ni2+-binding GTPase involved in regulation of | | CAETHG_3834 | CLJU_c17210 | * |
| | expression and maturation of urease and | | | | |
| | hydrogenase | | | | |
| 3095 | sulfonate transport system ATP-binding protein | | CAETHG_3939 | CLJU_c18320 | * |
| 3096 | sulfonate transport system permease protein | | CAETHG_3938 | CLJU_c18310 | * |
| 3097 | | | * | CLJU_c08790 | CLRAG_07700 |
| 3098 | nucleobase:cation symporter-1, NCS1 family | | CAETHG_3634 | CLJU_c15320 | * |
| 3099 | nucleobase:cation symporter-1, NCS1 family | | CAETHG_3637 | CLJU_c15350 | * |
| 3100 | Predicted nucleotidyltransferase | | CAETHG_3082 | CLJU_c09910 | * |
| 3101 | nucleotidyltransferase substrate binding protein, | | CAETHG_3083 | CLJU_c09920 | * |
| | HI0074 family | | | | |
| 3102 | Uncharacterized OsmC-related protein | | CAETHG_1871 | CLJU_c40250 | * |
| 3103 | hypothetical protein | | CAETHG_3168 | CLJU_c10780 | * |
| 3104 | DNA-binding transcriptional regulator, PadR family | | CAETHG_3715 | CLJU_c16210 | CLRAG_33120 |
| 3105 | CubicO group peptidase, beta-lactamase class C | | CAETHG_1716 | CLJU_c38660 | * |
| | family | | | | |
| 3106 | Transglycosylase | | CAETHG_3693 | CLJU_c15930 | * |
| 3107 | | | * | CLJU_c15530 | CLRAG_32610 |
| 3108 | | | * | CLJU_c22570 | CLRAG_32130 |
| 3109 | | | * | CLJU_c22560 | CLRAG_32120 |
| 3110 | | | * | CLJU_c22580, | CLRAG_32140 |
| | | | | CLJU_c22540 | |
| 3111 | | | * | CLJU_c22600 | CLRAG_32160 |
| 3112 | | | * | CLJU_c22590 | CLRAG_32150 |
| 3113 | hypothetical protein | | CAETHG_0948 | CLJU_c29540 | CLRAG_35530 |
| 3114 | rarD protein | | CAETHG_1170 | CLJU_c32400 | * |
| 3115 | hypothetical protein | | CAETHG_2070 | CLJU_c42450 | * |
| 3116 | putative ABC transport system permease protein | | CAETHG_2871 | CLJU_c07790 | * |
| 3117 | MFS transporter, DHA1 family, tetracycline | | CAETHG_3495 | CLJU_c14140 | * |
| | resistance protein | | | | |
| 3118 | | | * | CLJU_c31740 | CLRAG_02400 |
| 3119 | octaprenyl-diphosphate synthase | 2.5.1.29, | CAETHG_1877 | CLJU_c40310 | * |
| | | 2.5.1.1, | | | |
| | | 2.5.1.10 | | | |
| 3120 | hypothetical protein | | CAETHG_1875 | CLJU_c40290 | * |
| 3121 | serpin B | | CAETHG_0506 | CLJU_c24460 | * |
| 3122 | | | * | CLJU_c38400 | CLRAG_21860 |
| 3123 | Uncharacterized protein, pyridoxamine 5′- | | CAETHG_1566 | CLJU_c37110 | * |
| | phosphate oxidase (PNPOx-like) family | | | | |
| 3124 | small redox-active disulfide protein 2 | | CAETHG_0949 | CLJU_c29550 | CLRAG_35540 |
| 3125 | LuxR family transcriptional regulator, maltose | | CAETHG_0007 | CLJU_c19300 | * |
| | regulon positive regulatory protein | | | | |
| 3126 | MerR HTH family regulatory protein | | CAETHG_0781 | CLJU_c26970 | * |
| 3127 | DNA-binding transcriptional regulator, MerR family | | CAETHG_3503 | CLJU_c14210 | * |
| 3128 | two-component system, CitB family, response | | CAETHG_1703, | CLJU_c38470 | * |
| | regulator MalR | | CAETHG_2485 | | |
| 3129 | | | * | CLJU_c27170 | CLRAG_20060 |
| 3130 | two-component system, response regulator YcbB | | CAETHG_2071 | CLJU_c42460 | * |
| 3131 | two-component system, OmpR family, response | | CAETHG_0296 | CLJU_c21980, | * |
| | regulator VanR | | | CLJU_c14380 | |
| 3132 | RNA polymerase sigma-70 factor, ECF subfamily | | CAETHG_1322 | CLJU_c34230 | * |
| 3133 | protein of unknown function (DUF4179) | | CAETHG_1323 | CLJU_c34240 | * |
| 3134 | Methyltransferase domain-containing protein | | CAETHG_1536 | CLJU_c36260 | CLRAG_23930 |
| 3135 | two-component system, CitB family, sensor | | CAETHG_1704, | CLJU_c38480 | * |
| | histidine kinase MalK | | CAETHG_2484 | | |
| 3136 | His Kinase A (phospho-acceptor) domain- | | CAETHG_3648 | CLJU_c15470 | * |
| | containing protein | | | | |
| 3137 | Signal transduction histidine kinase | | CAETHG_2579 | CLJU_c05020 | CLRAG_38410 |
| 3138 | PAS domain S-box-containing protein | | CAETHG_0093, | CLJU_c20120, | CLRAG_29640 |
| | | | CAETHG_0101 | CLJU_c20200 | |
| 3139 | Signal transduction histidine kinase | | CAETHG_0957 | CLJU_c29610 | CLRAG_35600 |
| 3140 | Signal transduction histidine kinase | | CAETHG_2872 | CLJU_c07800 | * |
| 3141 | spermidine/putrescine transport system substrate- | | CAETHG_0260 | CLJU_c21730 | * |
| | binding protein | | | | |
| 3142 | spermidine/putrescine transport system permease | | CAETHG_0261 | CLJU_c21740 | * |
| | protein | | | | |
| 3143 | spermidine/putrescine transport system permease | | CAETHG_0262 | CLJU_c21750 | * |
| | protein | | | | |
| 3144 | spore coat protein | | CAETHG_0032 | CLJU_c19550 | * |
| 3145 | similar to spore coat protein | | CAETHG_0033 | CLJU_c19560 | * |
| 3146 | Coat F domain-containing protein | | CAETHG_0030 | CLJU_c19530 | * |
| 3147 | spore germination protein (amino acid permease) | | CAETHG_3655 | CLJU_c15600 | * |
| 3148 | spore germination protein KA | | CAETHG_2942 | CLJU_c08490 | * |
| 3149 | spore germination protein KC | | CAETHG_2943 | CLJU_c08500 | * |
| 3150 | spore germination protein KA | | CAETHG_3654 | CLJU_c15590 | * |
| 3151 | spore germination protein | | CAETHG_3656 | CLJU_c15610 | * |
| 3152 | Sugar phosphate isomerase/epimerase | | CAETHG_0868 | CLJU_c28730 | CLRAG_34810 |
| 3153 | glycoside/pentoside/hexuronide:cation symporter, | | CAETHG_0649 | CLJU_c25800 | * |
| | GPH family | | | | |
| 3154 | glycoside/pentoside/hexuronide:cation symporter, | | CAETHG_0655 | CLJU_c25860 | * |
| | GPH family | | | | |
| 3155 | sugar (Glycoside-Pentoside-Hexuronide) | | CAETHG_3437 | CLJU_c13530 | * |
| | transporter | | | | |
| 3156 | Ig-like domain (group 2) | | CAETHG_0334 | CLJU_c22340 | CLRAG_31930 |
| 3157 | Ig-like domain (group 2) | | CAETHG_0337 | CLJU_c22360 | CLRAG_32000 |
| 3158 | Putative intracellular protease/amidase | | CAETHG_1423 | CLJU_c35150 | * |
| 3159 | DNA-binding transcriptional regulator, MarR family | | CAETHG_0017 | CLJU_c19400 | * |
| 3160 | Predicted TIM-barrel enzyme | | CAETHG_0365 | CLJU_c23020 | * |
| 3161 | LuxR family transcriptional regulator, maltose | | CAETHG_0394 | CLJU_c23300 | * |
| | regulon positive regulatory protein | | | | |
| 3162 | DNA-binding transcriptional regulator, MocR | | CAETHG_0937 | CLJU_c29430 | CLRAG_35420 |
| | family, contains an aminotransferase domain | | | | |
| 3163 | ArsR family transcriptional regulator | | CAETHG_0950, | CLJU_c29560 | CLRAG_35550 |
| | | | CAETHG_3674 | | |
| 3164 | DNA-binding transcriptional regulator, MerR family | | CAETHG_1513 | CLJU_c36040 | * |
| 3165 | DNA-binding transcriptional regulator, XRE-family | | CAETHG_1707 | CLJU_c38540 | * |
| | HTH domain | | | | |
| 3166 | DNA-binding transcriptional regulator, MerR family | | CAETHG_1858 | CLJU_c40090 | * |
| 3167 | regulatory protein, Fis family | | CAETHG_2076, | CLJU_c42510, | * |
| | | | CAETHG_2078 | CLJU_c42520 | |
| 3168 | DNA-binding response regulator, OmpR family, | | CAETHG_2578 | CLJU_c05010 | CLRAG_38400 |
| | contains REC and winged-helix (wHTH) domain | | | | |
| 3169 | FCD domain-containing protein | | CAETHG_2773 | CLJU_c06820 | CLRAG_18620 |
| 3170 | Sugar-specific transcriptional regulator TrmB | | CAETHG_3027 | CLJU_c09320 | * |
| 3171 | DNA-binding transcriptional regulator, MarR family | | CAETHG_3494 | CLJU_c14130 | * |
| 3172 | DNA-binding transcriptional regulator, MerR family | | CAETHG_3594 | CLJU_c14880 | * |
| 3173 | DNA-binding transcriptional regulator, MarR family | | CAETHG_3874 | CLJU_c17660 | CLRAG_01070 |
| 3174 | GntR family transcriptional regulator/MocR | 2.6.1.23, | CAETHG_3893 | CLJU_c17850 | * |
| | family aminotransferase | 2.6.1.1 | | | |
| 3175 | | | * | CLJU_c35940 | CLRAG_06440 |
| 3176 | protein of unknown function (DUF4132) | | CAETHG_2741 | CLJU_c06450 | * |
| 3177 | HlyD family secretion protein | | CAETHG_0323 | CLJU_c22250 | CLRAG_31830 |
| 3178 | Sugar phosphate permease | | CAETHG_1781 | CLJU_c39360 | * |
| 3179 | Predicted arabinose efflux permease, MFS family | | CAETHG_1839 | CLJU_c39930 | * |
| 3180 | MFS transporter, SP family, inositol transporter | | CAETHG_2084 | CLJU_c42580 | * |
| 3181 | MFS transporter, ACS family, glucarate transporter | | CAETHG_2449 | CLJU_c03870 | * |
| 3182 | putative ABC transport system permease protein | | CAETHG_3516 | CLJU_c14430 | * |
| 3183 | | | * | CLJU_c22740 | CLRAG_02050 |
| 3184 | Histidine kinase-, DNA gyrase B-, and HSP90-like | | CAETHG_3514 | CLJU_c14400 | * |
| | ATPase | | | | |
| 3185 | DNA-binding response regulator, OmpR family, | | CAETHG_0956 | CLJU_c29600 | CLRAG_35590 |
| | contains REC and winged-helix (wHTH) domain | | | | |
| 3186 | DNA-binding response regulator, OmpR family, | | CAETHG_3647 | CLJU_c15460 | * |
| | contains REC and winged-helix (wHTH) domain | | | | |
| 3187 | Histidine kinase-, DNA gyrase B-, and HSP90-like | | CAETHG_3277 | CLJU_c11860 | * |
| | ATPase | | | | |
| 3188 | integrase/recombinase XerD | | CAETHG_3750 | CLJU_c16540 | CLRAG_33350 |
| 3189 | demethylmenaquinone methyltransferase/2- | 2.1.1.— | CAETHG_1876 | CLJU_c40300 | * |
| | methoxy-6-polyprenyl-1,4-benzoquinol methylase | | | | |
| 3190 | | | * | CLJU_c22750 | CLRAG_02040 |
| 3191 | uroporphyrinogen decarboxylase | | CAETHG_0197 | CLJU_c21110 | * |
| 3192 | Uroporphyrinogen decarboxylase (URO-D) | | CAETHG_0140 | CLJU_c20570 | * |
| 3193 | Vancomycin resistance protein YoaR, contains | | CAETHG_3566 | CLJU_c14670 | * |
| | peptidoglycan-binding and VanW domains | | | | |
| 3194 | aryl-alcohol dehydrogenase | 1.1.1.1 | CAETHG_3710 | CLJU_c16150, | * |
| | | | | CLJU_c38580 | |
| 3195 | pyridoxal 5′-phosphate synthase pdxS subunit | | CAETHG_1804 | CLJU_c39580 | * |
| 3196 | 5′-phosphate synthase pdxT subunit | | CAETHG_1805 | CLJU_c39590 | * |
| 3197 | | | * | CLJU_c24080 | CLRAG_30600 |
| 3198 | rhamnulose-1-phosphate aldolase | 4.1.2.19 | CAETHG_2085 | CLJU_c42590 | * |
| 3199 | | | * | CLJU_c00990 | CLRAG_19590 |
| 3200 | | | * | CLJU_c32540 | CLRAG_23690 |
| 3201 | shikimate dehydrogenase | 1.1.1.282, | CAETHG_0870 | CLJU_c28750 | CLRAG_34830 |
| | | 1.1.1.25 | | | |
| 3202 | shikimate kinase | | CAETHG_1641 | CLJU_c38140 | * |
| 3203 | spore germination protein KB | | CAETHG_2945 | CLJU_c08510 | * |
| 3204 | Tat proofreading chaperone TorD | | CAETHG_0615 | CLJU_c25460 | * |
| 3205 | DNA-binding transcriptional regulator, LysR family | | CAETHG_0612 | CLJU_c25430, | * |
| | | | | CLJU_c38560 | |
| 3206 | DNA-binding transcriptional regulator, MarR family | | CAETHG_0797 | CLJU_c27120 | * |
| 3207 | DNA-binding transcriptional regulator, LysR family | | CAETHG_0872 | CLJU_c28770 | CLRAG_34850 |
| 3208 | transcriptional regulator-like protein | | CAETHG_2927 | CLJU_c08320 | * |
| 3209 | transcriptional regulator, DeoR family | | CAETHG_0706 | CLJU_c26260 | * |
| 3210 | transcriptional regulator, DeoR family | | CAETHG_2088 | CLJU_c42620 | * |
| 3211 | transcriptional regulator, HxlR family | | CAETHG_0717, | CLJU_c26360, | * |
| | | | CAETHG_3595 | CLJU_c14890 | |
| 3212 | transcriptional regulator, HxlR family | | CAETHG_1567 | CLJU_c37120 | * |
| 3213 | transcriptional regulator, HxlR family | | CAETHG_3910 | CLJU_c18010 | * |
| 3214 | transcriptional regulator, MarR family with | | CAETHG_2176 | CLJU_c00580 | * |
| | acetyltransferase activity | | | | |
| 3215 | DNA-binding transcriptional regulator, PadR family | | CAETHG_1325, | CLJU_c34330, | * |
| | | | CAETHG_1333 | CLJU_c34260 | |
| 3216 | | | * | CLJU_c35130 | CLRAG_26380 |
| 3217 | transcriptional regulator, RpiR family | | CAETHG_3246 | CLJU_c11550 | * |
| 3218 | transcriptional regulator, TetR family | | CAETHG_1840 | CLJU_c39940 | * |
| 3219 | transcriptional regulator, TetR family | | CAETHG_2851 | CLJU_c07580 | CLRAG_32480 |
| 3220 | transcriptional regulator, TetR family | | CAETHG_2910 | CLJU_c08150 | * |
| 3221 | transketolase | 2.2.1.1 | CAETHG_0652 | CLJU_c25830 | * |
| 3222 | transketolase | 2.2.1.1 | CAETHG_0651 | CLJU_c25820 | * |
| 3223 | L-threonylcarbamoyladenylate synthase | | CAETHG_0779 | CLJU_c26950 | * |
| 3224 | Di- and tricarboxylate transporter | | CAETHG_0481 | CLJU_c24230 | * |
| 3225 | aldehyde:ferredoxin oxidoreductase | 1.2.7.5 | CAETHG_0092, | CLJU_c20110, | CLRAG_29650 |
| | | | CAETHG_0102 | CLJU_c20210 | |
| 3226 | AraC-type DNA-binding protein | | CAETHG_0656 | CLJU_c25870 | * |
| 3227 | Helix-turn-helix domain-containing protein | | CAETHG_3276 | CLJU_c11850 | * |
| 3228 | DNA-binding response regulator, OmpR family, | | CAETHG_0788 | CLJU_c27050 | * |
| | contains REC and winged-helix (wHTH) domain | | | | |
| 3229 | tyrosyl-tRNA synthetase | | CAETHG_1330 | CLJU_c34300 | * |
| 3230 | uroporphyrinogen decarboxylase | | CAETHG_4044 | CLJU_c19100 | * |
| 3231 | Uroporphyrinogen-III decarboxylase | | CAETHG_4043 | CLJU_c19090 | * |
| 3232 | UV-damage endonuclease | | CAETHG_3553 | CLJU_c14460 | * |
| 3233 | xylulokinase | 2.7.1.17 | CAETHG_3248 | CLJU_c11570 | * |
| 3234 | S-(hydroxymethyl)glutathione dehydrogenase/ | 1.1.1.1 | CAETHG_0031 | CLJU_c19540 | * |
| | alcohol dehydrogenase | | | | |
| 3235 | (R,R)-butanediol dehydrogenase/meso- | 1.1.1.1 | CAETHG_0650 | CLJU_c25810 | * |
| | butanediol dehydrogenase/diacetyl reductase | | | | |
| 3236 | (R,R)-butanediol dehydrogenase/meso- | 1.1.1.4 | CAETHG_0653 | CLJU_c25840 | * |
| | butanediol dehydrogenase/diacetyl reductase | | | | |
| 3237 | L-iditol 2-dehydrogenase | | CAETHG_3247 | CLJU_c11560 | * |
| 3238 | Molybdopterin oxidoreductase | 1.7.7.2, , | CAETHG_0085, | * | CLRAG_18830 |
| | | 1.2.1.43, | CAETHG_2789 | | |
| | | 1.1.99.33 | | | |
| 3239 | stage V sporulation protein AD | | CAETHG_0177 | * | CLRAG_19090 |
| 3240 | Spo0E like sporulation regulatory protein | | CAETHG_0289 | * | CLRAG_31470 |
| 3241 | hypothetical protein | | CAETHG_0626 | * | CLRAG_03780 |
| 3242 | hypothetical protein | | CAETHG_0703 | * | CLRAG_04380 |
| 3243 | hypothetical protein | | CAETHG_0704 | * | CLRAG_04390 |
| 3244 | hypothetical protein | | CAETHG_0705 | * | CLRAG_04400 |
| 3245 | hypothetical protein | | CAETHG_0927 | * | CLRAG_35350 |
| 3246 | hypothetical protein | | CAETHG_0945 | * | CLRAG_35500 |
| 3247 | hypothetical protein | | CAETHG_1024 | * | CLRAG_15770 |
| 3248 | GHKL domain-containing protein | | CAETHG_1025 | * | CLRAG_15780 |
| 3249 | two component transcriptional regulator, LytTR | | CAETHG_1026 | * | CLRAG_15790 |
| | family | | | | |
| 3250 | hypothetical protein | | CAETHG_1077, | * | CLRAG_16250 |
| | | | CAETHG_1086 | | |
| 3251 | Protein of unknown function (DUF1064) | | CAETHG_1093 | * | CLRAG_32410 |
| 3252 | hypothetical protein | | CAETHG_1511 | * | CLRAG_06530 |
| 3253 | hypothetical protein | | CAETHG_1637 | * | CLRAG_37490 |
| 3254 | DNA binding domain-containing protein, | | CAETHG_1666 | * | CLRAG_36100 |
| | excisionase family | | | | |
| 3255 | hypothetical protein | | CAETHG_1668 | * | CLRAG_36120 |
| 3256 | putative transcriptional regulator | | CAETHG_1669 | * | CLRAG_20580 |
| 3257 | DNA-binding transcriptional regulator, XRE-family | | CAETHG_1670 | * | CLRAG_20590 |
| | HTH domain | | | | |
| 3258 | Site-specific recombinase XerD | | CAETHG_1671 | * | CLRAG_20600 |
| 3259 | hypothetical protein | | CAETHG_1682 | * | CLRAG_20720 |
| 3260 | hypothetical protein | | CAETHG_1700 | * | CLRAG_20890 |
| 3261 | PadR family transcriptional regulator, regulatory | | CAETHG_1722 | * | CLRAG_20990 |
| | protein PadR | | | | |
| 3262 | Uncharacterized membrane-anchored protein | | CAETHG_1723 | * | CLRAG_21000 |
| 3263 | hypothetical protein | | CAETHG_2061 | * | CLRAG_05480 |
| 3264 | transcriptional regulator, RpiR family | | CAETHG_2187 | * | CLRAG_19880 |
| 3265 | hypothetical protein | | CAETHG_2338 | * | CLRAG_27930 |
| 3266 | Predicted transcriptional regulator containing CBS | | CAETHG_2437 | * | CLRAG_28830 |
| | domains | | | | |
| 3267 | hypothetical protein | | CAETHG_2668 | * | CLRAG_07000 |
| 3268 | NUDIX domain-containing protein | | CAETHG_2670 | * | CLRAG_30010 |
| 3269 | hypothetical protein | | CAETHG_2702 | * | CLRAG_07270 |
| 3270 | similar to stage IV sporulation protein | | CAETHG_2901 | * | CLRAG_08290 |
| 3271 | Putative motility protein | | CAETHG_3094 | * | CLRAG_13330 |
| 3272 | hypothetical protein | | CAETHG_3216 | * | CLRAG_12200 |
| 3273 | hypothetical protein | | CAETHG_3380 | * | CLRAG_10820 |
| 3274 | hypothetical protein | | CAETHG_3458 | * | CLRAG_10090 |
| 3275 | Signal transduction histidine kinase | | CAETHG_3575 | * | CLRAG_20390 |
| 3276 | Two-component response regulator, SAPR family, | | CAETHG_3576 | * | CLRAG_20380 |
| | consists of REC, wHTH and BTAD domains | | | | |
| 3277 | hypothetical protein | | CAETHG_3967 | * | CLRAG_00100 |
| 3278 | protein of unknown function (DUF1540) | | CAETHG_4060 | * | CLRAG_39820 |
| 3279 | | | * | CLJU_c03970 | * |
| 3280 | | | * | CLJU_c32730 | CLRAG_15600 |
| 3281 | | | * | CLJU_c27620 | * |
| 3282 | | | * | CLJU_c28050 | * |
| 3283 | | | * | CLJU_c27830, | * |
| | | | | CLJU_c27350 | |
| 3284 | CAAX protease self-immunity | | CAETHG_2013 | CLJU_c41840 | * |
| 3285 | | | * | CLJU_c27820, | * |
| | | | | CLJU_c27340 | |
| 3286 | | | * | CLJU_c14360 | * |
| 3287 | Predicted amidohydrolase | | CAETHG_1376 | CLJU_c34790 | * |
| 3288 | DNA-3-methyladenine glycosylase I | | CAETHG_1539 | CLJU_c36310 | * |
| 3289 | 2-iminobutanoate/2-iminopropanoate deaminase | | CAETHG_1883 | CLJU_c40370 | * |
| 3290 | flagellar hook-associated protein 2 | | CAETHG_3053 | CLJU_c09580 | * |
| 3291 | fumarate reductase flavoprotein subunit | | CAETHG_1032 | CLJU_c30250 | * |
| 3292 | | | * | CLJU_c28100 | * |
| 3293 | | | * | CLJU_c27840, | * |
| | | | | CLJU_c27360 | |
| 3294 | | | * | CLJU_c27770, | * |
| | | | | CLJU_c27290 | |
| 3295 | | | * | CLJU_c27780, | * |
| | | | | CLJU_c27300 | |
| 3296 | | | * | CLJU_c27790, | * |
| | | | | CLJU_c27310 | |
| 3297 | | | * | CLJU_c27490 | * |
| 3298 | | | * | CLJU_c27900 | * |
| 3299 | hypothetical protein | | CAETHG_0091 | CLJU_c20100 | * |
| 3300 | protein of unknown function (DUF4445) | | CAETHG_0194 | CLJU_c21090 | * |
| 3301 | hypothetical protein | | CAETHG_0784 | CLJU_c27000 | * |
| 3302 | Putative ABC-transporter type IV | | CAETHG_0808 | CLJU_c27230 | * |
| 3303 | hypothetical protein | | CAETHG_0810 | CLJU_c27240 | * |
| 3304 | hypothetical protein | | CAETHG_0818 | CLJU_c28180 | * |
| 3305 | hypothetical protein | | CAETHG_1009 | CLJU_c30100 | * |
| 3306 | hypothetical protein | | CAETHG_1010 | CLJU_c30110 | * |
| 3307 | PucR C-terminal helix-turn-helix domain- | | CAETHG_1028 | CLJU_c30210 | * |
| | containing protein | | | | |
| 3308 | Protein of unknown function (DUF1097) | | CAETHG_1030 | CLJU_c30230 | * |
| 3309 | hypothetical protein | | CAETHG_1036 | CLJU_c30290 | * |
| 3310 | Putative cell wall-binding protein | | CAETHG_1038 | CLJU_c30320 | * |
| 3311 | hypothetical protein | | CAETHG_1039 | CLJU_c30330 | * |
| 3312 | Microcystin-dependent protein | | CAETHG_1040 | CLJU_c30340 | * |
| 3313 | Acetyltransferase (GNAT) domain-containing | | CAETHG_1041 | CLJU_c30350 | * |
| | protein | | | | |
| 3314 | hypothetical protein | | CAETHG_1042 | CLJU_c30360 | * |
| 3315 | hypothetical protein | | CAETHG_1043 | CLJU_c30370 | * |
| 3316 | putative transport protein | | CAETHG_1049 | CLJU_c30440 | * |
| 3317 | N-terminal 7TM region of histidine kinase | 2.1.1.258 | CAETHG_1069 | CLJU_c30650 | * |
| 3318 | hypothetical protein | | CAETHG_1156 | CLJU_c32280 | * |
| 3319 | hypothetical protein | | CAETHG_1159 | CLJU_c32290 | * |
| 3320 | DnaD and phage-associated domain-containing | | CAETHG_1160 | CLJU_c32300 | * |
| | protein | | | | |
| 3321 | hypothetical protein | | CAETHG_1162 | CLJU_c32320 | * |
| 3322 | Putative zincin peptidase | | CAETHG_1169 | CLJU_c32390 | * |
| 3323 | hypothetical protein | | CAETHG_1328 | CLJU_c34290 | * |
| 3324 | PucR C-terminal helix-turn-helix domain- | | CAETHG_1369 | CLJU_c34720 | * |
| | containing protein | | | | |
| 3325 | hypothetical protein | | CAETHG_1377 | CLJU_c34800 | * |
| 3326 | hypothetical protein | | CAETHG_1445 | CLJU_c35360 | * |
| 3327 | Aminoacyl-tRNA editing domain-containing | | CAETHG_1452 | CLJU_c35440 | * |
| | protein | | | | |
| 3328 | hypothetical protein | | CAETHG_1489 | CLJU_c35810 | * |
| 3329 | EAL domain-containing protein | | CAETHG_1490 | CLJU_c35830 | * |
| 3330 | EAL domain-containing protein | | CAETHG_1491 | CLJU_c35840 | * |
| 3331 | hypothetical protein | | CAETHG_1732 | CLJU_c38840 | * |
| 3332 | hypothetical protein | | CAETHG_1850 | CLJU_c40030 | * |
| 3333 | Nuclease-related domain-containing protein | | CAETHG_1854 | CLJU_c40050 | * |
| 3334 | Uncharacterized membrane protein YeiH | | CAETHG_1880 | CLJU_c40340 | * |
| 3335 | phage regulatory protein, rha family | | CAETHG_2141 | CLJU_c00240 | * |
| 3336 | hypothetical protein | | CAETHG_2143 | CLJU_c00260 | * |
| 3337 | hypothetical protein | | CAETHG_2144 | CLJU_c00270 | * |
| 3338 | hypothetical protein | | CAETHG_2145 | CLJU_c00280 | * |
| 3339 | hypothetical protein | | CAETHG_2146 | CLJU_c00290 | * |
| 3340 | hypothetical protein | | CAETHG_2147 | CLJU_c00300, | * |
| | | | | CLJU_c36470 | |
| 3341 | hypothetical protein | | CAETHG_2148 | CLJU_c00310 | * |
| 3342 | hypothetical protein | | CAETHG_2150 | CLJU_c00330 | * |
| 3343 | hypothetical protein | | CAETHG_2151 | CLJU_c00340 | * |
| 3344 | hypothetical protein | | CAETHG_2153 | CLJU_c00360 | * |
| 3345 | hypothetical protein | | CAETHG_2154 | CLJU_c00370 | * |
| 3346 | hypothetical protein | | CAETHG_2156 | CLJU_c00380 | * |
| 3347 | hypothetical protein | | CAETHG_2157 | CLJU_c00390 | * |
| 3348 | hypothetical protein | | CAETHG_2159 | CLJU_c00410 | * |
| 3349 | hypothetical protein | | CAETHG_2160 | CLJU_c00420 | * |
| 3350 | transposase, IS605 OrfB family, central region | | CAETHG_2167 | CLJU_c00480 | * |
| 3351 | hypothetical protein | | CAETHG_2168 | CLJU_c00500 | * |
| 3352 | hypothetical protein | | CAETHG_2169 | CLJU_c00510 | * |
| 3353 | hypothetical protein | | CAETHG_2170 | CLJU_c00520 | * |
| 3354 | hypothetical protein | | CAETHG_2171 | CLJU_c00530 | * |
| 3355 | Uncharacterized conserved protein | | CAETHG_2172 | CLJU_c00540 | * |
| 3356 | hypothetical protein | | CAETHG_2737 | CLJU_c06410 | * |
| 3357 | hypothetical protein | | CAETHG_2805 | CLJU_c07130 | * |
| 3358 | hypothetical protein | | CAETHG_2842 | CLJU_c07490 | * |
| 3359 | hypothetical protein | | CAETHG_2858 | CLJU_c07650 | * |
| 3360 | protein FliT | | CAETHG_3054 | CLJU_c09590 | * |
| 3361 | Glycosyl transferases group 1 | | CAETHG_3068 | CLJU_c09750 | * |
| 3362 | hypothetical protein | | CAETHG_3081 | CLJU_c09900 | * |
| 3363 | hypothetical protein | | CAETHG_3084 | CLJU_c09930 | * |
| 3364 | carbamoyl-phosphate synthase large subunit | | CAETHG_3089 | CLJU_c09980 | * |
| 3365 | carbamoyl-phosphate synthase large subunit | | CAETHG_3090 | CLJU_c09990 | * |
| 3366 | hypothetical protein | | CAETHG_3175 | CLJU_c10860 | * |
| 3367 | Protein of unknown function (DUF2975) | | CAETHG_3486 | CLJU_c14050 | * |
| 3368 | Radical SAM superfamily enzyme YgiQ, UPF0313 | | CAETHG_3488 | CLJU_c14070 | * |
| | family | | | | |
| 3369 | ATP-binding cassette, subfamily B | | CAETHG_3489 | CLJU_c14080 | * |
| 3370 | heat shock protein DnaJ domain protein | | CAETHG_3554 | CLJU_c14470 | * |
| 3371 | HIRAN domain-containing protein | | CAETHG_3555 | CLJU_c14480 | * |
| 3372 | Acetyl esterase/lipase | | CAETHG_3596 | CLJU_c14900 | * |
| 3373 | hypothetical protein | | CAETHG_3616 | CLJU_c15140 | * |
| 3374 | protein of unknown function (DUF4179) | | CAETHG_3714 | CLJU_c16200 | * |
| 3375 | hypothetical protein | | CAETHG_3725 | CLJU_c16310 | * |
| 3376 | hypothetical protein | | CAETHG_3727 | CLJU_c16330 | * |
| 3377 | hypothetical protein | | CAETHG_3831 | CLJU_c17190 | * |
| 3378 | protein of unknown function (DUF4386) | | CAETHG_3875 | CLJU_c17670 | * |
| 3379 | Glucose inhibited division protein A | | CAETHG_3964 | CLJU_c18570 | * |
| 3380 | hypothetical protein | | CAETHG_4030 | CLJU_c18960 | * |
| 3381 | ABC transporter | | CAETHG_4034 | CLJU_c19000 | * |
| 3382 | Transposase and inactivated derivatives | | CAETHG_4054 | CLJU_c19190 | * |
| 3383 | | | * | CLJU_c27970 | * |
| 3384 | | | * | CLJU_c27920 | * |
| 3385 | | | * | CLJU_c27910 | * |
| 3386 | | | * | CLJU_c27880 | * |
| 3387 | | | * | CLJU_c27760 | * |
| 3388 | | | * | CLJU_c27750 | * |
| 3389 | | | * | CLJU_c27600 | * |
| 3390 | | | * | CLJU_c27590 | * |
| 3391 | | | * | CLJU_c16900 | * |
| 3392 | | | * | CLJU_c27370 | * |
| 3393 | | | * | CLJU_c27400 | * |
| 3394 | | | * | CLJU_c27420 | * |
| 3395 | | | * | CLJU_c27430 | * |
| 3396 | | | * | CLJU_c27460 | * |
| 3397 | | | * | CLJU_c27470 | * |
| 3398 | | | * | CLJU_c27500 | * |
| 3399 | | | * | CLJU_c27540 | * |
| 3400 | | | * | CLJU_c18820 | * |
| 3401 | | | * | CLJU_c18810 | * |
| 3402 | | | * | CLJU_c18800, | * |
| | | | | CLJU_c18780 | |
| 3403 | | | * | CLJU_c18790 | * |
| 3404 | | | * | CLJU_c18770 | * |
| 3405 | | | * | CLJU_c18710 | * |
| 3406 | | | * | CLJU_c30980 | * |
| 3407 | | | * | CLJU_c37150 | * |
| 3408 | | | * | CLJU_c28930 | * |
| 3409 | | | * | CLJU_c29510 | * |
| 3410 | | | * | CLJU_c15790 | * |
| 3411 | | | * | CLJU_c15950 | * |
| 3412 | | | * | CLJU_c31700 | * |
| 3413 | | | * | CLJU_c31690 | * |
| 3414 | | | * | CLJU_c31680 | * |
| 3415 | | | * | CLJU_c31670 | * |
| 3416 | | | * | CLJU_c31660 | * |
| 3417 | | | * | CLJU_c31650 | * |
| 3418 | | | * | CLJU_c31360 | * |
| 3419 | | | * | CLJU_c30940 | * |
| 3420 | | | * | CLJU_c36400 | * |
| 3421 | | | * | CLJU_c31730 | * |
| 3422 | | | * | CLJU_c31760 | * |
| 3423 | | | * | CLJU_c31780 | * |
| 3424 | | | * | CLJU_c14410 | * |
| 3425 | | | * | CLJU_c14620 | * |
| 3426 | | | * | CLJU_c15560 | * |
| 3427 | | | * | CLJU_c05760 | * |
| 3428 | | | * | CLJU_c10050 | * |
| 3429 | | | * | CLJU_c28140 | * |
| 3430 | | | * | CLJU_c27980 | * |
| 3431 | | | * | CLJU_c42660 | * |
| 3432 | | | * | CLJU_c23010 | * |
| 3433 | | | * | CLJU_c31370 | * |
| 3434 | | | * | CLJU_c31320 | * |
| 3435 | | | * | CLJU_c31300 | * |
| 3436 | | | * | CLJU_c31290 | * |
| 3437 | | | * | CLJU_c31280 | * |
| 3438 | | | * | CLJU_c31270 | * |
| 3439 | | | * | CLJU_c31260 | * |
| 3440 | | | * | CLJU_c31250 | * |
| 3441 | | | * | CLJU_c31240 | * |
| 3442 | | | * | CLJU_c31230 | * |
| 3443 | | | * | CLJU_c31210 | * |
| 3444 | | | * | CLJU_c31200 | * |
| 3445 | | | * | CLJU_c31190 | * |
| 3446 | | | * | CLJU_c31180 | * |
| 3447 | | | * | CLJU_c31170 | * |
| 3448 | | | * | CLJU_c31160 | * |
| 3449 | | | * | CLJU_c31130 | * |
| 3450 | | | * | CLJU_c31120 | * |
| 3451 | | | * | CLJU_c31110 | * |
| 3452 | | | * | CLJU_c31090 | * |
| 3453 | | | * | CLJU_c38410 | * |
| 3454 | | | * | CLJU_c08360 | * |
| 3455 | | | * | CLJU_c03680 | CLRAG_17070 |
| 3456 | | | * | CLJU_c09700 | CLRAG_13470 |
| 3457 | | | * | CLJU_c16600 | CLRAG_33500 |
| 3458 | | | * | CLJU_c16640 | CLRAG_33530 |
| 3459 | | | * | CLJU_c16650 | CLRAG_33540 |
| 3460 | | | * | CLJU_c16660 | CLRAG_33560 |
| 3461 | | | * | CLJU_c16680 | CLRAG_33580 |
| 3462 | | | * | CLJU_c16690 | CLRAG_33590 |
| 3463 | | | * | CLJU_c16700 | CLRAG_33600 |
| 3464 | | | * | CLJU_c18720 | CLRAG_16640 |
| 3465 | | | * | CLJU_c22370 | CLRAG_32010 |
| 3466 | | | * | CLJU_c22380 | CLRAG_32020 |
| 3467 | | | * | CLJU_c22400, | CLRAG_32040 |
| | | | | CLJU_c22430 | |
| 3468 | | | * | CLJU_c22410, | CLRAG_32050 |
| | | | | CLJU_c22440 | |
| 3469 | | | * | CLJU_c22460 | CLRAG_32070 |
| 3470 | | | * | CLJU_c22470 | CLRAG_32080 |
| 3471 | | | * | CLJU_c22550 | CLRAG_32110 |
| 3472 | | | * | CLJU_c22700 | CLRAG_02150 |
| 3473 | | | * | CLJU_c29580 | CLRAG_35570 |
| 3474 | | | * | CLJU_c31450 | CLRAG_16350 |
| 3475 | | | * | CLJU_c31570 | CLRAG_16390 |
| 3476 | | | * | CLJU_c32430 | CLRAG_03340 |
| 3477 | | | * | CLJU_c32490 | CLRAG_03370 |
| 3478 | | | * | CLJU_c32680 | CLRAG_29350 |
| 3479 | | | * | CLJU_c32690 | CLRAG_15640 |
| 3480 | | | * | CLJU_c32700 | CLRAG_15630 |
| 3481 | | | * | CLJU_c32710 | CLRAG_15620 |
| 3482 | | | * | CLJU_c32720 | CLRAG_15610 |
| 3483 | | | * | CLJU_c37770 | CLRAG_37190 |
| 3484 | | | * | CLJU_c37900 | CLRAG_37290 |
| 3485 | | | * | CLJU_c37950 | CLRAG_37340 |
| 3486 | | | * | CLJU_c37960 | CLRAG_37350 |
| 3487 | 4Fe—4S dicluster domain-containing protein | | CAETHG_2998 | CLJU_c09040 | * |
| 3488 | IstB-like ATP binding protein | | CAETHG_3079 | CLJU_c09880 | * |
| 3489 | | | * | CLJU_c27650 | * |
| 3490 | | | * | CLJU_c27380, | * |
| | | | | CLJU_c30970 | |
| 3491 | Multimeric flavodoxin WrbA | | CAETHG_1029 | CLJU_c30220 | * |
| 3492 | | | * | CLJU_c26180 | CLRAG_04260 |
| 3493 | peptide deformylase | | CAETHG_3721 | CLJU_c16270 | * |
| 3494 | | | * | CLJU_c03640 | * |
| 3495 | | | * | CLJU_c31220 | * |
| 3496 | Site-specific recombinase XerD | | CAETHG_3070 | CLJU_c09770 | * |
| 3497 | Homeodomain, phBC6A51-type | | CAETHG_2149 | CLJU_c00320 | * |
| 3498 | phage tail tape measure protein, TP901 family, | | CAETHG_2158 | CLJU_c00400 | * |
| | core region | | | | |
| 3499 | | | * | CLJU_c36480 | * |
| 3500 | | | * | CLJU_c31330 | * |
| 3501 | | | * | CLJU_c31310 | * |
| 3502 | | | * | CLJU_c31070 | * |
| 3503 | | | * | CLJU_c16810 | CLRAG_33720 |
| 3504 | | | * | CLJU_c38600 | * |
| 3505 | | | * | CLJU_c14340 | * |
| 3506 | exo-poly-alpha-galacturonosidase | | CAETHG_0930 | CLJU_c29360 | * |
| 3507 | hypothetical protein | | CAETHG_1050 | CLJU_c30450 | * |
| 3508 | | | * | CLJU_c38080 | CLRAG_37450 |
| 3509 | maltose O-acetyltransferase | | CAETHG_0520 | CLJU_c24570 | * |
| 3510 | putative acetyltransferase | | CAETHG_1163 | CLJU_c32330 | * |
| 3511 | | | * | CLJU_c27630 | * |
| 3512 | | | * | CLJU_c37730 | CLRAG_37150 |
| 3513 | | | * | CLJU_c37740 | CLRAG_37160 |
| 3514 | | | * | CLJU_c32660 | CLRAG_29360 |
| 3515 | | | * | CLJU_c27610 | * |
| 3516 | | | * | CLJU_c27950 | * |
| 3517 | | | * | CLJU_c31080 | * |
| 3518 | | | * | CLJU_c30310 | * |
| 3519 | 2-hydroxycarboxylate transporter family protein | | CAETHG_0599 | CLJU_c25300 | * |
| 3520 | MFS transporter, CP family, cyanate transporter | | CAETHG_3728 | CLJU_c16340 | * |
| 3521 | RraA family | | CAETHG_1374 | CLJU_c34770 | * |
| 3522 | | | * | CLJU_c37720 | CLRAG_37140 |
| 3523 | | | * | CLJU_c37750 | CLRAG_37170 |
| 3524 | | | * | CLJU_c12090 | * |
| 3525 | | | * | CLJU_c12040 | * |
| 3526 | | | * | CLJU_c12080 | * |
| 3527 | | | * | CLJU_c12070 | * |
| 3528 | | | * | CLJU_c12060 | * |
| 3529 | | | * | CLJU_c12050 | * |
| 3530 | putative transcriptional regulator | | CAETHG_2140 | CLJU_c00230 | * |
| 3531 | | | * | CLJU_c16610 | CLRAG_33510 |
| 3532 | | | * | CLJU_c32480 | CLRAG_03360 |
| 3533 | | | * | CLJU_c32510 | CLRAG_03390 |
| 3534 | Fur family transcriptional regulator, ferric uptake | | CAETHG_3722 | CLJU_c16280 | * |
| | regulator | | | | |
| 3535 | | | * | CLJU_c37710 | CLRAG_37130 |
| 3536 | flagellin N-terminal helical region | | CAETHG_3092 | CLJU_c10010 | * |
| 3537 | flagellin | | CAETHG_3065 | CLJU_c09710 | * |
| 3538 | flagellin | | CAETHG_3055 | CLJU_c09600 | * |
| 3539 | | | * | CLJU_c37820 | CLRAG_37240 |
| 3540 | General stress protein 26 | | CAETHG_1154 | CLJU_c32260 | * |
| 3541 | | | * | CLJU_c15570 | * |
| 3542 | Glycosyltransferase involved in cell wall | | CAETHG_2599 | CLJU_c05220 | * |
| | bisynthesis | | | | |
| 3543 | Glycosyl transferases group 1 | | CAETHG_2602 | CLJU_c05230 | * |
| 3544 | hypothetical protein | | CAETHG_2604 | CLJU_c05250 | * |
| 3545 | Glycosyltransferase, GT2 family | | CAETHG_4032 | CLJU_c18980 | * |
| 3546 | | | * | CLJU_c14310 | * |
| 3547 | | | * | CLJU_c27410 | * |
| 3548 | Phage integrase family protein | | CAETHG_3072 | CLJU_c09790 | * |
| 3549 | | | * | CLJU_c38030 | CLRAG_37400 |
| 3550 | | | * | CLJU_c38040 | CLRAG_37410 |
| 3551 | | | * | CLJU_c37220 | * |
| 3552 | DNA-binding transcriptional regulator, LysR family | | CAETHG_0635 | CLJU_c25660 | * |
| 3553 | DNA-binding transcriptional regulator, LysR family | | CAETHG_1372 | CLJU_c34750 | * |
| 3554 | | | * | CLJU_c37910 | CLRAG_37300 |
| 3555 | DNA-binding transcriptional regulator, MarR family | | CAETHG_3724 | CLJU_c16300 | * |
| 3556 | GHKL domain-containing protein | | CAETHG_3730 | CLJU_c16360 | * |
| 3557 | hypothetical protein | | CAETHG_1007 | CLJU_c30090 | * |
| 3558 | Helix-turn-helix | | CAETHG_2014 | CLJU_c41850 | * |
| 3559 | | | * | CLJU_c38710 | * |
| 3560 | | | * | CLJU_c28070 | * |
| 3561 | | | * | CLJU_c37860 | CLRAG_37250 |
| 3562 | | | * | CLJU_c38000, | CLRAG_37370 |
| | | | | CLJU_c38020 | |
| 3563 | | | * | CLJU_c16030 | * |
| 3564 | Na+/H+ antiporter NhaC | | CAETHG_1537 | CLJU_c36290 | * |
| 3565 | methyl-accepting chemotaxis protein | | CAETHG_3723 | CLJU_c16290 | * |
| 3566 | | | * | CLJU_c28030 | * |
| 3567 | | | * | CLJU_c15520 | CLRAG_32600 |
| 3568 | Methyltransferase domain-containing protein | | CAETHG_1164 | CLJU_c32340 | * |
| 3569 | | | * | CLJU_c22690 | CLRAG_02160 |
| 3570 | | | * | CLJU_c28120 | CLRAG_09080 |
| 3571 | | | * | CLJU_c38100 | CLRAG_37470 |
| 3572 | | | * | CLJU_c38090 | CLRAG_37460 |
| 3573 | | | * | CLJU_c27930 | * |
| 3574 | Nucleoside-diphosphate-sugar epimerase | | CAETHG_0073 | CLJU_c19930 | * |
| 3575 | NADPH-dependent glutamate synthase beta chain | | CAETHG_1033 | CLJU_c30260 | * |
| 3576 | | | * | CLJU_c38670 | * |
| 3577 | | | * | CLJU_c22630 | CLRAG_02220 |
| 3578 | | | * | CLJU_c22640 | CLRAG_02210 |
| 3579 | | | * | CLJU_c22670 | CLRAG_02180 |
| 3580 | | | * | CLJU_c22650 | CLRAG_02200 |
| 3581 | | | * | CLJU_c22660 | CLRAG_02190 |
| 3582 | | | * | CLJU_c27860 | * |
| 3583 | | | * | CLJU_c37160 | * |
| 3584 | | | * | CLJU_c37870, | CLRAG_37260 |
| | | | | CLJU_c37890 | |
| 3585 | Transcriptional regulator, contains XRE-family HTH | | CAETHG_2139 | CLJU_c00220 | * |
| | domain | | | | |
| 3586 | | | * | CLJU_c31380 | * |
| 3587 | phage-like integrase | | CAETHG_3071 | CLJU_c09780 | * |
| 3588 | Phage integrase family protein | | CAETHG_3073 | CLJU_c09800 | * |
| 3589 | Phage integrase, N-terminal SAM-like domain | | CAETHG_3074 | CLJU_c09810 | * |
| 3590 | | | * | CLJU_c32470 | CLRAG_03350 |
| 3591 | Phage integrase family protein | | CAETHG_0071 | CLJU_c19910 | * |
| 3592 | | | * | CLJU_c03690 | CLRAG_17060 |
| 3593 | phage terminase small subunit | | CAETHG_2152 | CLJU_c00350 | * |
| 3594 | hypothetical protein | | CAETHG_1031 | CLJU_c30240 | * |
| 3595 | undecaprenyl-diphosphatase | | CAETHG_3807 | CLJU_c16970 | * |
| 3596 | D-3-phosphoglycerate dehydrogenase | | CAETHG_1373 | CLJU_c34760 | * |
| 3597 | | | * | CLJU_c32650 | CLRAG_29370 |
| 3598 | | | * | CLJU_c27940 | * |
| 3599 | | | * | CLJU_c27960 | * |
| 3600 | DNA-binding transcriptional regulator, MerR family | | CAETHG_0072 | CLJU_c19920 | * |
| 3601 | | | * | CLJU_c27580 | * |
| 3602 | | | * | CLJU_c27510 | * |
| 3603 | PAS domain S-box-containing protein | | CAETHG_0094, | CLJU_c24140, | * |
| | | | CAETHG_0472 | CLJU_c24060, | |
| | | | | CLJU_c20130 | |
| 3604 | | | * | CLJU_c21760 | * |
| 3605 | spore germination protein KC | | CAETHG_1735 | CLJU_c38870 | * |
| 3606 | spore germination protein KA | | CAETHG_1733 | CLJU_c38850 | * |
| 3607 | Spore germination protein | | CAETHG_1734 | CLJU_c38860 | * |
| 3608 | | | * | CLJU_c38390 | * |
| 3609 | | | * | CLJU_c22420, | CLRAG_32060 |
| | | | | CLJU_c22450 | |
| 3610 | DNA-binding transcriptional regulator, MarR family | | CAETHG_0719 | CLJU_c26380 | * |
| 3611 | PAS domain S-box-containing protein | | CAETHG_1884 | CLJU_c40410 | * |
| 3612 | DNA-binding transcriptional regulator, MarR family | | CAETHG_3726 | CLJU_c16320 | * |
| 3613 | | | * | CLJU_c27390 | * |
| 3614 | | | * | CLJU_c15550 | * |
| 3615 | | | * | CLJU_c07090, | CLRAG_02170 |
| | | | | CLJU_c22680 | |
| 3616 | Sugar phosphate permease | | CAETHG_1375 | CLJU_c34780 | * |
| 3617 | | | * | CLJU_c37430 | * |
| 3618 | | | * | CLJU_c37420 | * |
| 3619 | Transposase DDE domain-containing protein | | CAETHG_0888 | CLJU_c28920 | * |
| 3620 | Transposase | | CAETHG_3075 | CLJU_c09820 | * |
| 3621 | hypothetical protein | | CAETHG_3078 | CLJU_c09860 | * |
| 3622 | | | * | CLJU_c38690 | * |
| 3623 | | | * | CLJU_c14350 | * |
| 3624 | | | * | CLJU_c29570 | CLRAG_35560 |
| 3625 | RNA polymerase sigma-70 factor, ECF subfamily | | CAETHG_3713 | CLJU_c16190 | * |
| 3626 | | | * | CLJU_c27730 | * |
| 3627 | | | * | CLJU_c14370 | * |
| 3628 | | | * | CLJU_c27800, | * |
| | | | | CLJU_c27320 | |
| 3629 | | | * | CLJU_c27810, | * |
| | | | | CLJU_c27330 | |
| 3630 | threonine synthase | 4.2.3.1, | CAETHG_1882 | CLJU_c40360 | * |
| | | 4.2.99.2 | | | |
| 3631 | Glycosyltransferase involved in cell wall | | CAETHG_3067 | CLJU_c09740 | * |
| | bisynthesis | | | | |
| 3632 | transcriptional regulator of aroF, aroG, tyrA and | | CAETHG_1538 | CLJU_c36300 | * |
| | aromatic amino acid transport | | | | |
| 3633 | | | * | CLJU_c28060, | * |
| | | | | CLJU_c28040 | |
| 3634 | DNA-binding transcriptional regulator, GntR family | | CAETHG_1056 | CLJU_c30510 | * |
| 3635 | transcriptional regulator, HxlR family | | CAETHG_1153 | CLJU_c32250 | * |
| 3636 | DNA-binding transcriptional regulator, MarR family | | CAETHG_3719 | CLJU_c16250 | * |
| 3637 | | | * | CLJU_c27850 | * |
| 3638 | | | * | CLJU_c38610 | * |
| 3639 | | | * | CLJU_c14330 | * |
| 3640 | | | * | CLJU_c26190 | CLRAG_04270 |
| 3641 | | | * | CLJU_c12020 | * |
| 3642 | transporter, NhaC family | | CAETHG_1881 | CLJU_c40350 | * |
| 3643 | Di- and tricarboxylate transporter | | CAETHG_0600 | CLJU_c25310 | * |
| 3644 | two component transcriptional regulator, LytTR | | CAETHG_1006 | CLJU_c30080 | * |
| | family | | | | |
| 3645 | two component transcriptional regulator, LytTR | | CAETHG_3731 | CLJU_c16370 | * |
| | family | | | | |
| 3646 | uroporphyrinogen decarboxylase | | CAETHG_1037 | CLJU_c30300 | * |
| 3647 | hypothetical protein | | CAETHG_0137 | * | * |
| 3648 | hypothetical protein | | CAETHG_0195 | * | * |
| 3649 | hypothetical protein | | CAETHG_0270 | * | * |
| 3650 | ABC transporter ATP-binding protein | | CAETHG_0294 | * | * |
| 3651 | hypothetical protein | | CAETHG_0453 | * | * |
| 3652 | elongation factor with tetracycline resistance | 6.3.5.5 | CAETHG_0514 | * | * |
| | domain containing protein | | | | |
| 3653 | hypothetical protein | 2.7.7.22 | CAETHG_0516 | * | * |
| 3654 | hypothetical protein | | CAETHG_0524 | * | * |
| 3655 | hypothetical protein | | CAETHG_0528 | * | * |
| 3656 | hypothetical protein | | CAETHG_0549 | * | * |
| 3657 | Nicotinamidase-related amidase | 3.3.2.1 | CAETHG_0695 | * | * |
| 3658 | hypothetical protein | | CAETHG_0701 | * | * |
| 3659 | hypothetical protein | | CAETHG_0763 | * | * |
| 3660 | hypothetical protein | | CAETHG_0793 | * | * |
| 3661 | Transposase InsO and inactivated derivatives | | CAETHG_0814, | * | * |
| | | | CAETHG_2268, | | |
| | | | CAETHG_1100 | | |
| 3662 | transposase | | CAETHG_0815, | * | * |
| | | | CAETHG_2269, | | |
| | | | CAETHG_1101 | | |
| 3663 | Na+:H+ antiporter, NhaA family | | CAETHG_0953 | * | CLRAG_21450 |
| 3664 | HxlR family transcriptional regulator | | CAETHG_0960 | * | * |
| 3665 | hypothetical protein | 1.3.99.1 | CAETHG_1022 | * | CLRAG_15760 |
| 3666 | CRISPR-associated protein Cas2 | | CAETHG_1394 | * | * |
| 3667 | CRISP-associated protein Cas1 | | CAETHG_1395 | * | * |
| 3668 | CRISPR-associated exonuclease Cas4 | | CAETHG_1396 | * | * |
| 3669 | hypothetical protein | | CAETHG_1434 | * | * |
| 3670 | carbon-monoxide dehydrogenase catalytic subunit | | CAETHG_1620 | * | * |
| 3671 | hypothetical protein | | CAETHG_1636 | * | * |
| 3672 | ATP-dependent DNA helicase RecG | | CAETHG_1648 | * | CLRAG_29890 |
| 3673 | PilZ domain-containing protein | | CAETHG_1696 | * | CLRAG_20850 |
| 3674 | hypothetical protein | | CAETHG_2261 | * | * |
| 3675 | PAS domain S-box-containing protein | | CAETHG_2512 | * | CLRAG_37760 |
| 3676 | 2,3-dimethylmalate lyase | | CAETHG_2513 | * | CLRAG_37770 |
| 3677 | Sugar phosphate permease | | CAETHG_2514 | * | CLRAG_37780 |
| 3678 | YgiT-type zinc finger domain-containing protein | | CAETHG_2556 | * | * |
| 3679 | REP element-mobilizing transposase RayT | | CAETHG_2561 | * | CLRAG_38230 |
| 3680 | hypothetical protein | | CAETHG_2562 | * | * |
| 3681 | WxcM-like, C-terminal | | CAETHG_2608 | * | * |
| 3682 | dTDP-4-amino-4,6-dideoxygalactose transaminase | | CAETHG_2609 | * | * |
| 3683 | mannose-1-phosphate guanylyltransferase/ | | CAETHG_2636 | * | CLRAG_38780 |
| | mannose-6-phosphate isomerase | | | | |
| 3684 | type IV pilus assembly protein PilA | | CAETHG_2644 | * | CLRAG_06750 |
| 3685 | type II secretion system protein E (GspE) | | CAETHG_2645 | * | CLRAG_06760 |
| 3686 | type IV pilus assembly protein PilC | | CAETHG_2646 | * | CLRAG_06770 |
| 3687 | type IV pilus assembly protein PilM | | CAETHG_2647 | * | CLRAG_06780 |
| 3688 | type IV pilus assembly protein PilO | | CAETHG_2649 | * | CLRAG_06790 |
| 3689 | prepilin-type N-terminal cleavage/methylation | | CAETHG_2650 | * | CLRAG_06800 |
| | domain-containing protein | | | | |
| 3690 | hypothetical protein | | CAETHG_2651 | * | CLRAG_06810 |
| 3691 | prepilin-type N-terminal cleavage/methylation | | CAETHG_2652 | * | CLRAG_06820 |
| | domain-containing protein | | | | |
| 3692 | PilX N-terminal | | CAETHG_2653 | * | CLRAG_06830 |
| 3693 | putative esterase | | CAETHG_2672 | * | * |
| 3694 | Purple acid Phosphatase, N-terminal domain | | CAETHG_2695 | * | * |
| 3695 | methyl-accepting chemotaxis sensory transducer | | CAETHG_2856 | * | * |
| 3696 | hypothetical protein | | CAETHG_2944 | * | * |
| 3697 | hypothetical protein | | CAETHG_3435 | * | * |
| 3698 | ATP-dependent helicase IRC3 | | CAETHG_3519 | * | CLRAG_09710 |
| 3699 | hypothetical protein | | CAETHG_3521 | * | CLRAG_09730 |
| 3700 | hypothetical protein | | CAETHG_3522 | * | CLRAG_09740 |
| 3701 | AAA domain (dynein-related subfamily) | | CAETHG_3525 | * | * |
| 3702 | hypothetical protein | | CAETHG_3526 | * | * |
| 3703 | hypothetical protein | | CAETHG_3538 | * | CLRAG_20350 |
| 3704 | DNA repair exonuclease SbcCD nuclease subunit | | CAETHG_3542 | * | CLRAG_09860 |
| 3705 | Uncharacterized protein YhaN | | CAETHG_3543 | * | CLRAG_09870 |
| 3706 | PD-(D/E)XK nuclease superfamily protein | | CAETHG_3544 | * | CLRAG_09670 |
| 3707 | hypothetical protein | | CAETHG_3546 | * | CLRAG_09690 |
| 3708 | ATP-dependent helicase IRC3 | | CAETHG_3547 | * | * |
| 3709 | hypothetical protein | | CAETHG_3549 | * | * |
| 3710 | hypothetical protein | | CAETHG_3598 | * | * |
| 3711 | DNA-binding transcriptional regulator, MarR family | | CAETHG_3658 | * | CLRAG_32640 |
| 3712 | Methyltransferase domain-containing protein | | CAETHG_3659 | * | CLRAG_32650 |
| 3713 | phosphinothricin acetyltransferase | | CAETHG_3660 | * | CLRAG_32660 |
| 3714 | C_GCAxxG_C_C family probable redox protein | | CAETHG_3661 | * | CLRAG_32670 |
| 3715 | DGC domain-containing protein | | CAETHG_3668 | * | CLRAG_32750 |
| 3716 | DGC domain-containing protein | | CAETHG_3669 | * | CLRAG_32760 |
| 3717 | glycine cleavage system H protein | | CAETHG_3670 | * | CLRAG_32770 |
| 3718 | NAD-dependent dihydropyrimidine | | CAETHG_3671 | * | CLRAG_32780 |
| | dehydrogenase, PreA subunit | | | | |
| 3719 | hypothetical protein | | CAETHG_3672 | * | CLRAG_32790 |
| 3720 | hypothetical protein | | CAETHG_3673 | * | CLRAG_32810 |
| 3721 | zinc-dependent peptidase | | CAETHG_3676 | * | CLRAG_32840 |
| 3722 | DNA-binding transcriptional regulator, XRE-family | | CAETHG_3752 | * | * |
| | HTH domain | | | | |
| 3723 | hypothetical protein | | CAETHG_3753 | * | CLRAG_33400 |
| 3724 | hypothetical protein | | CAETHG_3754 | * | CLRAG_33410 |
| 3725 | DNA segregation ATPase FtsK/SpoIIIE, S-DNA-T | | CAETHG_3755 | * | CLRAG_33420 |
| | family | | | | |
| 3726 | restriction system protein | | CAETHG_3757 | * | CLRAG_33440 |
| 3727 | DNA helicase-2/ATP-dependent DNA helicase | | CAETHG_3803 | * | CLRAG_33820 |
| | PcrA | | | | |
| 3728 | hypothetical protein | | CAETHG_3810 | * | * |
| 3729 | arsenical pump membrane protein | | CAETHG_3985 | * | CLRAG_16620 |
| 3730 | protein of unknown function (DUF3794) | | CAETHG_3987 | * | CLRAG_16610 |
| 3731 | hypothetical protein | | CAETHG_3988 | * | CLRAG_16600 |
| 3732 | hypothetical protein | | CAETHG_3989, | * | CLRAG_16590 |
| | | | CAETHG_3990 | | |
| 3733 | hypothetical protein | | CAETHG_3991 | * | CLRAG_16570 |
| 3734 | Spo0E like sporulation regulatory protein | | CAETHG_3995 | * | CLRAG_16530 |
| 3735 | Cupin domain-containing protein | 5.3.1.8 | CAETHG_3997 | * | CLRAG_16510 |
| 3736 | autoinducer 2 (AI-2) kinase | | CAETHG_3998 | * | CLRAG_16500 |
| 3737 | DNA-binding transcriptional regulator LsrR, DeoR | | CAETHG_3999 | * | CLRAG_16490 |
| | family | | | | |
| 3738 | AI-2 transport system ATP-binding protein | | CAETHG_4000 | * | CLRAG_16480 |
| 3739 | AI-2 transport system permease protein | | CAETHG_4001 | * | CLRAG_16470 |
| 3740 | AI-2 transport system permease protein | | CAETHG_4002 | * | CLRAG_16460 |
| 3741 | AI-2 transport system substrate-binding protein | | CAETHG_4003 | * | CLRAG_16450 |
| 3742 | putative autoinducer-2 (AI-2) aldolase | | CAETHG_4004 | * | CLRAG_16440 |
| 3743 | hypothetical protein | | CAETHG_4005 | * | CLRAG_16430 |
| 3744 | Glycosyltransferase, GT2 family | | CAETHG_4007, | * | CLRAG_16410 |
| | | | CAETHG_4012 | | |
| 3745 | Peptidoglycan/xylan/chitin deacetylase, | | CAETHG_4008 | * | CLRAG_40260 |
| | PgdA/CDA1 family | | | | |
| 3746 | UDPglucose 6-dehydrogenase | 1.1.1.22 | CAETHG_4009 | * | CLRAG_40250 |
| 3747 | Glycosyltransferase like family 2 | | CAETHG_4010 | * | CLRAG_40230 |
| 3748 | Glycosyl transferase family 2 | | CAETHG_4011, | * | CLRAG_40210 |
| | | | CAETHG_4013, | | |
| | | | CAETHG_4014 | | |
| 3749 | Spore maturation protein CgeB | | CAETHG_4015 | * | CLRAG_40170 |
| 3750 | hypothetical protein | | CAETHG_4016 | * | CLRAG_40160 |
| 3751 | hypothetical protein | | CAETHG_4021 | * | CLRAG_40100 |
| 3752 | hypothetical protein | | CAETHG_4022 | * | CLRAG_40090 |
| 3753 | | | * | * | CLRAG_38840 |
| 3754 | | | * | * | CLRAG_39170 |
| 3755 | | | * | * | CLRAG_39160 |
| 3756 | | | * | * | CLRAG_39140 |
| 3757 | | | * | * | CLRAG_39110 |
| 3758 | | | * | * | CLRAG_39100 |
| 3759 | | | * | * | CLRAG_29850 |
| 3760 | | | * | * | CLRAG_33000 |
| 3761 | | | * | * | CLRAG_33280 |
| 3762 | | | * | * | CLRAG_33290 |
| 3763 | | | * | * | CLRAG_40220 |
| 3764 | | | * | * | CLRAG_17980 |
| 3765 | | | * | * | CLRAG_03540 |
| 3766 | | | * | * | CLRAG_06340 |
| 3767 | | | * | * | CLRAG_06490 |
| 3768 | | | * | * | CLRAG_09680 |
| 3769 | | | * | * | CLRAG_32280 |
| 3770 | | | * | * | CLRAG_32270 |
| 3771 | | | * | * | CLRAG_32400 |
| 3772 | | | * | * | CLRAG_32390 |
| 3773 | | | * | * | CLRAG_32380 |
| 3774 | | | * | * | CLRAG_32370 |
| 3775 | | | * | * | CLRAG_24810 |
| 3776 | | | * | * | CLRAG_30620 |
| 3777 | | | * | * | CLRAG_30760 |
| 3778 | | | * | * | CLRAG_32420 |
| 3779 | | | * | * | CLRAG_01000 |
| 3780 | | | * | * | CLRAG_00960 |
| 3781 | | | * | * | CLRAG_02850 |
| 3782 | | | * | * | CLRAG_02860 |
| 3783 | | | * | * | CLRAG_02870 |
| 3784 | | | * | * | CLRAG_02880 |
| 3785 | | | * | * | CLRAG_02900 |
| 3786 | | | * | * | CLRAG_02910 |
| 3787 | | | * | * | CLRAG_02920 |
| 3788 | | | * | * | CLRAG_02950 |
| 3789 | | | * | * | CLRAG_02960 |
| 3790 | | | * | * | CLRAG_02970 |
| 3791 | | | * | * | CLRAG_02980 |
| 3792 | | | * | * | CLRAG_02990 |
| 3793 | | | * | * | CLRAG_03000 |
| 3794 | | | * | * | CLRAG_03010 |
| 3795 | | | * | * | CLRAG_03310 |
| 3796 | | | * | * | CLRAG_03330 |
| 3797 | | | * | * | CLRAG_15190 |
| 3798 | | | * | * | CLRAG_15180 |
| 3799 | | | * | * | CLRAG_09910 |
| 3800 | | | * | * | CLRAG_32560 |
| 3801 | | | * | * | CLRAG_30530 |
| 3802 | | | * | * | CLRAG_32700 |
| 3803 | | | * | * | CLRAG_14810 |
| 3804 | | | * | * | CLRAG_25230 |
| 3805 | | | * | * | CLRAG_25290 |
| 3806 | | | * | * | CLRAG_20440 |
| 3807 | | | * | * | CLRAG_37570 |
| 3808 | | | * | * | CLRAG_26360 |
| 3809 | | | * | * | CLRAG_37610 |
| 3810 | | | * | * | CLRAG_36020 |
| 3811 | | | * | * | CLRAG_36040 |
| 3812 | | | * | * | CLRAG_36050 |
| 3813 | | | * | * | CLRAG_36060 |
| 3814 | | | * | * | CLRAG_09130 |
| 3815 | | | * | * | CLRAG_05640 |
| 3816 | | | * | * | CLRAG_16370 |
| 3817 | | | * | * | CLRAG_23850 |
| 3818 | | | * | * | CLRAG_13360 |
| 3819 | | | * | * | CLRAG_13380 |
| 3820 | | | * | CLJU_c01630, | * |
| | | | | CLJU_c38500, | |
| | | | | CLJU_c37040, | |
| | | | | CLJU_c31010, | |
| | | | | CLJU_c17540 | |
| 3821 | | | * | CLJU_c14540 | * |
| 3822 | | | * | CLJU_c15870 | * |
| 3823 | | | * | CLJU_c03760 | * |
| 3824 | | | * | CLJU_c37840 | * |
| 3825 | | | * | CLJU_c00490 | * |
| 3826 | | | * | CLJU_c01550 | * |
| 3827 | | | * | CLJU_c01590 | * |
| 3828 | | | * | CLJU_c02010 | * |
| 3829 | | | * | CLJU_c02510 | * |
| 3830 | | | * | CLJU_c03280 | * |
| 3831 | | | * | CLJU_c03290 | * |
| 3832 | | | * | CLJU_c03300 | * |
| 3833 | | | * | CLJU_c03340 | * |
| 3834 | | | * | CLJU_c03350 | * |
| 3835 | | | * | CLJU_c03360 | * |
| 3836 | | | * | CLJU_c03410 | * |
| 3837 | | | * | CLJU_c03450 | * |
| 3838 | | | * | CLJU_c03460 | * |
| 3839 | | | * | CLJU_c03490 | * |
| 3840 | | | * | CLJU_c03500 | * |
| 3841 | | | * | CLJU_c03510 | * |
| 3842 | | | * | CLJU_c03570 | * |
| 3843 | | | * | CLJU_c03580 | * |
| 3844 | | | * | CLJU_c03590 | * |
| 3845 | | | * | CLJU_c03600 | * |
| 3846 | | | * | CLJU_c03630 | * |
| 3847 | | | * | CLJU_c03700 | * |
| 3848 | | | * | CLJU_c03730 | * |
| 3849 | | | * | CLJU_c03750 | * |
| 3850 | | | * | CLJU_c03790 | * |
| 3851 | | | * | CLJU_c03800 | * |
| 3852 | | | * | CLJU_c04140 | * |
| 3853 | | | * | CLJU_c04260 | * |
| 3854 | | | * | CLJU_c04540 | * |
| 3855 | | | * | CLJU_c04850 | * |
| 3856 | | | * | CLJU_c05260 | * |
| 3857 | | | * | CLJU_c05360 | * |
| 3858 | | | * | CLJU_c05370 | * |
| 3859 | | | * | CLJU_c05590 | * |
| 3860 | | | * | CLJU_c05810 | * |
| 3861 | | | * | CLJU_c06880 | * |
| 3862 | | | * | CLJU_c07740 | * |
| 3863 | | | * | CLJU_c08420 | * |
| 3864 | | | * | CLJU_c08940 | * |
| 3865 | | | * | CLJU_c09720 | * |
| 3866 | | | * | CLJU_c09850 | * |
| 3867 | | | * | CLJU_c10030 | * |
| 3868 | | | * | CLJU_c10790 | * |
| 3869 | | | * | CLJU_c10810 | * |
| 3870 | | | * | CLJU_c10890 | * |
| 3871 | | | * | CLJU_c12030 | * |
| 3872 | | | * | CLJU_c12660 | * |
| 3873 | | | * | CLJU_c12760 | * |
| 3874 | | | * | CLJU_c13500 | * |
| 3875 | | | * | CLJU_c13510 | * |
| 3876 | | | * | CLJU_c13590 | * |
| 3877 | | | * | CLJU_c14040 | * |
| 3878 | | | * | CLJU_c14440 | * |
| 3879 | | | * | CLJU_c14490 | * |
| 3880 | | | * | CLJU_c14500 | * |
| 3881 | | | * | CLJU_c14510 | * |
| 3882 | | | * | CLJU_c14520 | * |
| 3883 | | | * | CLJU_c14530 | * |
| 3884 | | | * | CLJU_c14550 | * |
| 3885 | | | * | CLJU_c14560 | * |
| 3886 | | | * | CLJU_c14570 | * |
| 3887 | | | * | CLJU_c14580 | * |
| 3888 | | | * | CLJU_c14590 | * |
| 3889 | | | * | CLJU_c14600 | * |
| 3890 | | | * | CLJU_c14610 | * |
| 3891 | | | * | CLJU_c14690 | * |
| 3892 | | | * | CLJU_c14760 | * |
| 3893 | | | * | CLJU_c14830 | * |
| 3894 | | | * | CLJU_c15030 | * |
| 3895 | | | * | CLJU_c15040 | * |
| 3896 | | | * | CLJU_c15390 | * |
| 3897 | | | * | CLJU_c15850 | * |
| 3898 | | | * | CLJU_c15880 | * |
| 3899 | | | * | CLJU_c15890 | * |
| 3900 | | | * | CLJU_c15900 | * |
| 3901 | | | * | CLJU_c15970 | * |
| 3902 | | | * | CLJU_c15980 | * |
| 3903 | | | * | CLJU_c16010 | * |
| 3904 | | | * | CLJU_c16180 | * |
| 3905 | | | * | CLJU_c16560 | * |
| 3906 | | | * | CLJU_c16570 | * |
| 3907 | | | * | CLJU_c16620 | * |
| 3908 | | | * | CLJU_c16630 | * |
| 3909 | | | * | CLJU_c16670 | * |
| 3910 | | | * | CLJU_c16710 | * |
| 3911 | | | * | CLJU_c16730 | * |
| 3912 | | | * | CLJU_c16740 | * |
| 3913 | | | * | CLJU_c16750 | * |
| 3914 | | | * | CLJU_c16760 | * |
| 3915 | | | * | CLJU_c16780 | * |
| 3916 | | | * | CLJU_c16790 | * |
| 3917 | | | * | CLJU_c16800 | * |
| 3918 | | | * | CLJU_c16820 | * |
| 3919 | | | * | CLJU_c16850 | * |
| 3920 | | | * | CLJU_c16860 | * |
| 3921 | | | * | CLJU_c16870 | * |
| 3922 | | | * | CLJU_c16880 | * |
| 3923 | | | * | CLJU_c16890 | * |
| 3924 | | | * | CLJU_c16910 | * |
| 3925 | | | * | CLJU_c16920 | * |
| 3926 | | | * | CLJU_c18170 | * |
| 3927 | | | * | CLJU_c18840 | * |
| 3928 | | | * | CLJU_c22480 | * |
| 3929 | | | * | CLJU_c22490 | * |
| 3930 | | | * | CLJU_c23350 | * |
| 3931 | | | * | CLJU_c24820 | * |
| 3932 | | | * | CLJU_c27010 | * |
| 3933 | | | * | CLJU_c27870 | * |
| 3934 | | | * | CLJU_c28150 | * |
| 3935 | | | * | CLJU_c30070 | * |
| 3936 | | | * | CLJU_c30180 | * |
| 3937 | | | * | CLJU_c30190 | * |
| 3938 | | | * | CLJU_c30380 | * |
| 3939 | | | * | CLJU_c30570 | * |
| 3940 | | | * | CLJU_c30580 | * |
| 3941 | | | * | CLJU_c30830 | * |
| 3942 | | | * | CLJU_c30840 | * |
| 3943 | | | * | CLJU_c30910 | * |
| 3944 | | | * | CLJU_c31060 | * |
| 3945 | | | * | CLJU_c31100 | * |
| 3946 | | | * | CLJU_c31140 | * |
| 3947 | | | * | CLJU_c31150 | * |
| 3948 | | | * | CLJU_c31350 | * |
| 3949 | | | * | CLJU_c31410 | * |
| 3950 | | | * | CLJU_c31420 | * |
| 3951 | | | * | CLJU_c31430 | * |
| 3952 | | | * | CLJU_c31440 | * |
| 3953 | | | * | CLJU_c31470 | * |
| 3954 | | | * | CLJU_c31500 | * |
| 3955 | | | * | CLJU_c31520 | * |
| 3956 | | | * | CLJU_c31530 | * |
| 3957 | | | * | CLJU_c31560 | * |
| 3958 | | | * | CLJU_c31580 | * |
| 3959 | | | * | CLJU_c31770 | * |
| 3960 | | | * | CLJU_c32440 | * |
| 3961 | | | * | CLJU_c32450 | * |
| 3962 | | | * | CLJU_c32460 | * |
| 3963 | | | * | CLJU_c32590 | * |
| 3964 | | | * | CLJU_c32600 | * |
| 3965 | | | * | CLJU_c32610 | * |
| 3966 | | | * | CLJU_c32620 | * |
| 3967 | | | * | CLJU_c32670 | * |
| 3968 | | | * | CLJU_c33460 | * |
| 3969 | | | * | CLJU_c35370 | * |
| 3970 | | | * | CLJU_c35820 | * |
| 3971 | | | * | CLJU_c35990 | * |
| 3972 | | | * | CLJU_c36270 | * |
| 3973 | | | * | CLJU_c36280 | * |
| 3974 | | | * | CLJU_c36380 | * |
| 3975 | | | * | CLJU_c36390 | * |
| 3976 | | | * | CLJU_c36420 | * |
| 3977 | | | * | CLJU_c36430 | * |
| 3978 | | | * | CLJU_c36490 | * |
| 3979 | | | * | CLJU_c36540 | * |
| 3980 | | | * | CLJU_c36580 | * |
| 3981 | | | * | CLJU_c36590 | * |
| 3982 | | | * | CLJU_c36600 | * |
| 3983 | | | * | CLJU_c36610 | * |
| 3984 | | | * | CLJU_c36630 | * |
| 3985 | | | * | CLJU_c36640 | * |
| 3986 | | | * | CLJU_c37980 | * |
| 3987 | | | * | CLJU_c37990 | * |
| 3988 | | | * | CLJU_c38570 | * |
| 3989 | | | * | CLJU_c38730 | * |
| 3990 | | | * | CLJU_c38750 | * |
| 3991 | | | * | CLJU_c38760 | * |
| 3992 | | | * | CLJU_c39050 | * |
| 3993 | | | * | CLJU_c40150 | * |
| 3994 | | | * | CLJU_c40170 | * |
| 3995 | | | * | CLJU_c40190 | * |
| 3996 | | | * | CLJU_c40380 | * |
| 3997 | | | * | CLJU_c40400 | * |
| 3998 | | | * | CLJU_c42670 | * |
| 3999 | | | * | CLJU_c14630 | * |
| 4000 | | | * | CLJU_c30860 | * |
| 4001 | | | * | CLJU_c05280 | * |
| 4002 | | | * | CLJU_c05240 | * |
| 4003 | | | * | CLJU_c16840 | * |
| 4004 | | | * | CLJU_c03390, | * |
| | | | | CLJU_c36550 | |
| 4005 | | | * | CLJU_c03550 | * |
| 4006 | | | * | CLJU_c03530 | * |
| 4007 | | | * | CLJU_c03370 | * |
| 4008 | | | * | CLJU_c03380, | * |
| | | | | CLJU_c36560 | |
| 4009 | | | * | CLJU_c03420 | * |
| 4010 | | | * | CLJU_c03430, | * |
| | | | | CLJU_c36520 | |
| 4011 | | | * | CLJU_c03560 | * |
| 4012 | | | * | CLJU_c03650 | * |
| 4013 | | | * | CLJU_c03660 | * |
| 4014 | | | * | CLJU_c03670 | * |
| 4015 | | | * | CLJU_c16720 | * |
| 4016 | | | * | CLJU_c16770 | * |
| 4017 | | | * | CLJU_c31460 | * |
| 4018 | | | * | CLJU_c31480 | * |
| 4019 | | | * | CLJU_c31590 | * |
| 4020 | | | * | CLJU_c31630 | * |
| 4021 | | | * | CLJU_c31640 | * |
| 4022 | | | * | CLJU_c36530 | * |
| 4023 | | | * | CLJU_c36570 | * |
| 4024 | | | * | CLJU_c27680 | * |
| 4025 | | | * | CLJU_c22500 | * |
| 4026 | | | * | CLJU_c27660 | * |
| 4027 | | | * | CLJU_c28970 | * |
| 4028 | | | * | CLJU_c28980 | * |
| 4029 | | | * | CLJU_c28990 | * |
| 4030 | | | * | CLJU_c01650, | * |
| | | | | CLJU_c38520, | |
| | | | | CLJU_c37060, | |
| | | | | CLJU_c31030, | |
| | | | | CLJU_c17520 | |
| 4031 | | | * | CLJU_c30170 | * |
| 4032 | | | * | CLJU_c16830 | * |
| 4033 | | | * | CLJU_c14770 | * |
| 4034 | | | * | CLJU_c36450 | * |
| 4035 | | | * | CLJU_c37830 | * |
| 4036 | | | * | CLJU_c18950 | * |
| 4037 | | | * | CLJU_c30990 | * |
| 4038 | | | * | CLJU_c36410 | * |
| 4039 | | | * | CLJU_c31510 | * |
| 4040 | | | * | CLJU_c30850 | * |
| 4041 | | | * | CLJU_c37850 | * |
| 4042 | | | * | CLJU_c05270 | * |
| 4043 | | | * | CLJU_c05290 | * |
| 4044 | | | * | CLJU_c05300 | * |
| 4045 | | | * | CLJU_c09730 | * |
| 4046 | | | * | CLJU_c36460 | * |
| 4047 | | | * | CLJU_c32630 | * |
| 4048 | | | * | CLJU_c14020 | * |
| 4049 | | | * | CLJU_c14030 | * |
| 4050 | | | * | CLJU_c25560, | * |
| | | | | CLJU_c28490, | |
| | | | | CLJU_c27520 | |
| 4051 | | | * | CLJU_c38740 | * |
| 4052 | | | * | CLJU_c27670 | * |
| 4053 | | | * | CLJU_c28020 | * |
| 4054 | | | * | CLJU_c03480 | * |
| 4055 | | | * | CLJU_c22530 | * |
| 4056 | | | * | CLJU_c27550 | * |
| 4057 | | | * | CLJU_c15830 | * |
| 4058 | | | * | CLJU_c03780 | * |
| 4059 | | | * | CLJU_c15860 | * |
| 4060 | | | * | CLJU_c03610 | * |
| 4061 | | | * | CLJU_c36680 | * |
| 4062 | | | * | CLJU_c03540 | * |
| 4063 | | | * | CLJU_c36650 | * |
| 4064 | | | * | CLJU_c03740 | * |
| 4065 | | | * | CLJU_c03440, | * |
| | | | | CLJU_c36500 | |
| 4066 | | | * | CLJU_c31610 | * |
| 4067 | | | * | CLJU_c03520 | * |
| 4068 | | | * | CLJU_c31400 | * |
| 4069 | | | * | CLJU_c31390 | * |
| 4070 | | | * | CLJU_c31620 | * |
| 4071 | | | * | CLJU_c03470 | * |
| 4072 | | | * | CLJU_c03620 | * |
| 4073 | | | * | CLJU_c03720 | * |
| 4074 | | | * | CLJU_c31490 | * |
| 4075 | | | * | CLJU_c36440 | * |
| 4076 | | | * | CLJU_c16580 | * |
| 4077 | | | * | CLJU_c30870 | * |
| 4078 | | | * | CLJU_c03400 | * |
| 4079 | | | * | CLJU_c03770 | * |
| 4080 | | | * | CLJU_c36620 | * |
| 4081 | | | * | CLJU_c01620, | * |
| | | | | CLJU_c38490, | |
| | | | | CLJU_c37030, | |
| | | | | CLJU_c31000, | |
| | | | | CLJU_c17550 | |
| 4082 | | | * | CLJU_c39040 | * |
| 4083 | | | * | CLJU_c22390 | * |
| 4084 | | | * | CLJU_c31550 | * |
| 4085 | | | * | CLJU_c31710 | * |
| 4086 | | | * | CLJU_c31720 | * |
| 4087 | | | * | CLJU_c36660 | * |
| 4088 | | | * | CLJU_c36670 | * |
| 4089 | | | * | CLJU_c09870 | * |
| 4090 | | | * | CLJU_c37080 | * |
| 4091 | | | * | CLJU_c01640, | * |
| | | | | CLJU_c38510, | |
| | | | | CLJU_c37050, | |
| | | | | CLJU_c31020, | |
| | | | | CLJU_c17530 | |
| 4092 | | | * | CLJU_c03310 | * |
| 4093 | | | * | CLJU_c28530 | * |
| 4094 | | | * | CLJU_c39030 | * |
| 4095 | | | * | CLJU_c39060, | * |
| | | | | CLJU_c39070 | |
| 4096 | | | * | CLJU_c01660, | * |
| | | | | CLJU_c38530, | |
| | | | | CLJU_c37070, | |
| | | | | CLJU_c31040, | |
| | | | | CLJU_c17510 | |
| 4097 | | | * | CLJU_c03330 | * |
| 4098 | | | * | CLJU_c03320 | * |
| 4099 | | | * | CLJU_c15920 | * |
| 4100 | | | * | CLJU_c36510 | * |
| 4101 | tripeptide aminopeptidase | | CAETHG_0006 | * | * |
| 4102 | hypothetical protein | | CAETHG_0053 | * | * |
| 4103 | Response regulator receiver domain-containing | | CAETHG_0055 | * | * |
| | protein | | | | |
| 4104 | exonuclease SbcC | | CAETHG_0114 | * | * |
| 4105 | methyl-accepting chemotaxis protein | | CAETHG_0120 | * | * |
| 4106 | hypothetical protein | | CAETHG_0152 | * | * |
| 4107 | 2Fe—2S iron-sulfur cluster binding domain- | | CAETHG_0157 | * | * |
| | containing protein | | | | |
| 4108 | Tetratricopeptide repeat-containing protein | | CAETHG_0236 | * | * |
| 4109 | Glycerophosphoryl diester phosphodiesterase | | CAETHG_0268 | * | * |
| | family protein | | | | |
| 4110 | Leucyl aminopeptidase (aminopeptidase T) | | CAETHG_0279 | * | * |
| 4111 | protein AroM | | CAETHG_0280 | * | * |
| 4112 | hypothetical protein | | CAETHG_0282 | * | * |
| 4113 | Uncharacterized membrane protein, oligopeptide | | CAETHG_0283 | * | * |
| | transporter (OPT) family | | | | |
| 4114 | purine catabolism regulatory protein | | CAETHG_0284 | * | * |
| 4115 | hypothetical protein | | CAETHG_0325 | * | * |
| 4116 | hypothetical protein | | CAETHG_0326 | * | * |
| 4117 | hypothetical protein | | CAETHG_0331 | * | * |
| 4118 | hypothetical protein | | CAETHG_0336 | * | * |
| 4119 | AcrB/AcrD/AcrF family protein | | CAETHG_0392 | * | * |
| 4120 | hypothetical protein | | CAETHG_0399 | * | * |
| 4121 | MFS transporter, NNP family, nitrate/nitrite | | CAETHG_0439 | * | * |
| | transporter | | | | |
| 4122 | hypothetical protein | | CAETHG_0489 | * | * |
| 4123 | hypothetical protein | | CAETHG_0494 | * | * |
| 4124 | hypothetical protein | | CAETHG_0519 | * | * |
| 4125 | hypothetical protein | | CAETHG_0547 | * | * |
| 4126 | hypothetical protein | | CAETHG_0548 | * | * |
| 4127 | hypothetical protein | | CAETHG_0567 | * | * |
| 4128 | ATP-dependent DNA helicase RecQ | | CAETHG_0595 | * | * |
| 4129 | hypothetical protein | | CAETHG_0624 | * | * |
| 4130 | hypothetical protein | | CAETHG_0632 | * | * |
| 4131 | endoglucanase | | CAETHG_0687 | * | * |
| 4132 | hypothetical protein | | CAETHG_0688 | * | * |
| 4133 | hypothetical protein | | CAETHG_0689 | * | * |
| 4134 | SNF2 family N-terminal domain-containing protein | | CAETHG_0690 | * | * |
| 4135 | SNF2 family N-terminal domain-containing protein | | CAETHG_0691 | * | * |
| 4136 | radical SAM additional 4Fe4S-binding SPASM | | CAETHG_0692 | * | * |
| | domain-containing protein | | | | |
| 4137 | hypothetical protein | | CAETHG_0693 | * | * |
| 4138 | hypothetical protein | | CAETHG_0694, | * | * |
| | | | CAETHG_3528 | | |
| 4139 | epoxyqueuosine reductase | | CAETHG_0713 | * | * |
| 4140 | hypothetical protein | | CAETHG_0766 | * | * |
| 4141 | hypothetical protein | | CAETHG_0790 | * | * |
| 4142 | hypothetical protein | | CAETHG_0798 | * | * |
| 4143 | hypothetical protein | | CAETHG_0809 | * | * |
| 4144 | PD-(D/E)XK nuclease superfamily protein | | CAETHG_0837 | * | * |
| 4145 | cyclic lactone autoinducer peptide | | CAETHG_0845 | * | * |
| 4146 | hypothetical protein | | CAETHG_0851 | * | * |
| 4147 | hypothetical protein | | CAETHG_0877 | * | * |
| 4148 | Uncharacterized membrane protein YcaP, DUF421 | | CAETHG_0951 | * | * |
| | family | | | | |
| 4149 | hypothetical protein | | CAETHG_0952 | * | * |
| 4150 | putative transposase | | CAETHG_0954 | * | * |
| 4151 | esterase | | CAETHG_0962 | * | * |
| 4152 | GDSL-like Lipase/Acylhydrolase family protein | | CAETHG_0963 | * | * |
| 4153 | PhoH-like ATPase | | CAETHG_0970 | * | * |
| 4154 | GHKL domain-containing protein | | CAETHG_1008 | * | * |
| 4155 | hypothetical protein | | CAETHG_1011 | * | * |
| 4156 | hypothetical protein | | CAETHG_1017 | * | * |
| 4157 | hypothetical protein | | CAETHG_1018 | * | * |
| 4158 | hypothetical protein | | CAETHG_1019 | * | * |
| 4159 | hypothetical protein | | CAETHG_1020 | * | * |
| 4160 | Superfamily II DNA or RNA helicase | | CAETHG_1021 | * | * |
| 4161 | hypothetical protein | | CAETHG_1023 | * | * |
| 4162 | methyl-accepting chemotaxis protein | | CAETHG_1061 | * | * |
| 4163 | Transglutaminase-like superfamily protein | | CAETHG_1087 | * | * |
| 4164 | Putative cell wall-binding protein | | CAETHG_1088 | * | * |
| 4165 | hypothetical protein | | CAETHG_1094 | * | * |
| 4166 | protein of unknown function (DUF4868) | | CAETHG_1095 | * | * |
| 4167 | hypothetical protein | | CAETHG_1096 | * | * |
| 4168 | hypothetical protein | | CAETHG_1099 | * | * |
| 4169 | Predicted secreted protein | | CAETHG_1102 | * | * |
| 4170 | Fis1 C-terminal tetratricopeptide repeat- | | CAETHG_1104 | * | * |
| | containing protein | | | | |
| 4171 | phage-like protein | | CAETHG_1105 | * | * |
| 4172 | hypothetical protein | | CAETHG_1106 | * | * |
| 4173 | hypothetical protein | | CAETHG_1107 | * | * |
| 4174 | hypothetical protein | | CAETHG_1157 | * | * |
| 4175 | hypothetical protein | | CAETHG_1158 | * | * |
| 4176 | hypothetical protein | | CAETHG_1213 | * | * |
| 4177 | hypothetical protein | | CAETHG_1233 | * | * |
| 4178 | capD, capsular polysaccharide biosynthesis protein | | CAETHG_1316 | * | * |
| 4179 | tyrosyl-tRNA synthetase | | CAETHG_1329 | * | * |
| 4180 | hypothetical protein | | CAETHG_1362 | * | * |
| 4181 | hypothetical protein | | CAETHG_1378 | * | * |
| 4182 | CRISPR-associated endonuclease/helicase Cas3 | | CAETHG_1397 | * | * |
| 4183 | CRISPR-associated protein Cas5h | | CAETHG_1398 | * | * |
| 4184 | CRISPR-associated protein Csh2 | | CAETHG_1399 | * | * |
| 4185 | CRISPR-associated protein Csh1 | | CAETHG_1400 | * | * |
| 4186 | CRISPR-associated endoribonuclease Cas6 | | CAETHG_1401 | * | * |
| 4187 | hypothetical protein | | CAETHG_1402, | * | * |
| | | | CAETHG_1403 | | |
| 4188 | hypothetical protein | | CAETHG_1404 | * | * |
| 4189 | hypothetical protein | | CAETHG_1405 | * | * |
| 4190 | hypothetical protein | | CAETHG_1406 | * | * |
| 4191 | hypothetical protein | | CAETHG_1507 | * | * |
| 4192 | uroporphyrinogen-III decarboxylase-like protein | | CAETHG_1520 | * | * |
| 4193 | Four helix bundle sensory module for signal | | CAETHG_1530 | * | * |
| | transduction | | | | |
| 4194 | iron-dependent hydrogenase | | CAETHG_1575 | * | * |
| 4195 | NADH-quinone oxidoreductase subunit G | | CAETHG_1576 | * | * |
| 4196 | NADH-quinone oxidoreductase subunit E | | CAETHG_1578 | * | * |
| 4197 | hypothetical protein | | CAETHG_1640 | * | * |
| 4198 | hypothetical protein | | CAETHG_1642 | * | * |
| 4199 | hypothetical protein | | CAETHG_1643 | * | * |
| 4200 | hypothetical protein | | CAETHG_1645 | * | * |
| 4201 | hypothetical protein | | CAETHG_1646 | * | * |
| 4202 | hypothetical protein | | CAETHG_1647 | * | * |
| 4203 | PLD-like domain-containing protein | | CAETHG_1649 | * | * |
| 4204 | Adenine-specific DNA methylase, contains a Zn- | | CAETHG_1650 | * | * |
| | ribbon domain | | | | |
| 4205 | Protein of unknown function (DUF3780) | | CAETHG_1651 | * | * |
| 4206 | Chitobiase/beta-hexosaminidase C-terminal | | CAETHG_1652 | * | * |
| | domain-containing protein | | | | |
| 4207 | conserved hypothetical protein | | CAETHG_1653 | * | * |
| 4208 | hypothetical protein | | CAETHG_1659 | * | * |
| 4209 | hypothetical protein | | CAETHG_1660 | * | * |
| 4210 | hypothetical protein | | CAETHG_1661 | * | * |
| 4211 | hypothetical protein | | CAETHG_1662 | * | * |
| 4212 | hypothetical protein | | CAETHG_1663 | * | * |
| 4213 | hypothetical protein | | CAETHG_1664 | * | * |
| 4214 | aspartate kinase | | CAETHG_1689 | * | * |
| 4215 | transcriptional regulator, TetR family | | CAETHG_1705 | * | * |
| 4216 | hypothetical protein | | CAETHG_1706 | * | * |
| 4217 | regulatory protein, luxR family | | CAETHG_1709 | * | * |
| 4218 | hypothetical protein | | CAETHG_1710 | * | * |
| 4219 | Peptidase family M28 | | CAETHG_1711 | * | * |
| 4220 | chloramphenicol O-acetyltransferase type A | | CAETHG_1717 | * | * |
| 4221 | Helix-turn-helix | | CAETHG_1724 | * | * |
| 4222 | hypothetical protein | | CAETHG_1752 | * | * |
| 4223 | endonuclease-3 | | CAETHG_1772 | * | * |
| 4224 | hypothetical protein | | CAETHG_1803 | * | * |
| 4225 | putative ABC transport system permease protein | | CAETHG_1845 | * | * |
| 4226 | hypothetical protein | | CAETHG_1852 | * | * |
| 4227 | hypothetical protein | | CAETHG_1853 | * | * |
| 4228 | hypothetical protein | | CAETHG_1922 | * | * |
| 4229 | hypothetical protein | | CAETHG_2012 | * | * |
| 4230 | hypothetical protein | | CAETHG_2030 | * | * |
| 4231 | [acyl-carrier-protein] S-malonyltransferase | | CAETHG_2047 | * | * |
| 4232 | regulatory protein, Fis family | | CAETHG_2077 | * | * |
| 4233 | hypothetical protein | | CAETHG_2155 | * | * |
| 4234 | hypothetical protein | | CAETHG_2164 | * | * |
| 4235 | Protein of unknown function (DUF1015) | | CAETHG_2212 | * | * |
| 4236 | hypothetical protein | | CAETHG_2225, | * | * |
| | | | CAETHG_2727 | | |
| 4237 | GntR family transcriptional regulator, arabinose | | CAETHG_2232 | * | * |
| | operon transcriptional repressor | | | | |
| 4238 | hypothetical protein | | CAETHG_2238 | * | * |
| 4239 | hypothetical protein | | CAETHG_2280 | * | * |
| 4240 | spore coat protein, CotS family | | CAETHG_2303 | * | * |
| 4241 | hypothetical protein | | CAETHG_2305 | * | * |
| 4242 | spore coat protein | | CAETHG_2307 | * | * |
| 4243 | hypothetical protein | | CAETHG_2361 | * | * |
| 4244 | hypothetical protein | | CAETHG_2371 | * | * |
| 4245 | Transposase | | CAETHG_2386 | * | * |
| 4246 | benzoate membrane transport protein | | CAETHG_2388 | * | * |
| 4247 | nicotinate-nucleotide pyrophosphorylase | 2.4.2.19 | CAETHG_2389 | * | * |
| | (carboxylating) | | | | |
| 4248 | molybdenum cofactor synthesis domain- | | CAETHG_2390 | * | * |
| | containing protein | | | | |
| 4249 | 2-keto-4-pentenoate hydratase | | CAETHG_2391 | * | * |
| 4250 | BFD-like [2Fe—2S] binding domain-containing | | CAETHG_2392 | * | * |
| | protein | | | | |
| 4251 | hypothetical protein | | CAETHG_2393 | * | * |
| 4252 | Protein of unknown function DUF111 | | CAETHG_2394 | * | * |
| 4253 | hypothetical protein | | CAETHG_2395 | * | * |
| 4254 | transcriptional regulator, IclR family | | CAETHG_2396 | * | * |
| 4255 | uncharacterized protein | | CAETHG_2397 | * | * |
| 4256 | 4-hydroxy-tetrahydrodipicolinate synthase | 4.2.1.52 | CAETHG_2398 | * | * |
| 4257 | Four helix bundle sensory module for signal transduction | | CAETHG_2399 | * | * |
| 4258 | methyl-accepting chemotaxis protein | | CAETHG_2400 | * | * |
| 4259 | Pyruvate kinase, barrel domain | | CAETHG_2440 | * | * |
| 4260 | Collagen triple helix repeat-containing protein | | CAETHG_2494 | * | * |
| 4261 | carbamoyl-phosphate synthase small subunit | | CAETHG_2509 | * | * |
| 4262 | hypothetical protein | | CAETHG_2526 | * | * |
| 4263 | putative ABC transport system ATP-binding protein | | CAETHG_2559 | * | * |
| 4264 | putative ABC transport system permease protein | | CAETHG_2560 | * | * |
| 4265 | glycosyltransferase | | CAETHG_2600 | * | * |
| 4266 | hypothetical protein | | CAETHG_2601 | * | * |
| 4267 | O-antigen ligase like membrane protein | | CAETHG_2603 | * | * |
| 4268 | hypothetical protein | | CAETHG_2605 | * | * |
| 4269 | Glycosyltransferase, GT2 family | | CAETHG_2606 | * | * |
| 4270 | transferase hexapeptide (six repeat-containing protein) | | CAETHG_2607 | * | * |
| 4271 | Membrane protein involved in the export of O-antigen and teichoic | | CAETHG_2610 | * | * |
| | acid | | | | |
| 4272 | Glycosyltransferase involved in cell wall | | CAETHG_2611 | * | * |
| | bisynthesis | | | | |
| 4273 | hypothetical protein | | CAETHG_2612 | * | * |
| 4274 | Glycosyltransferase involved in cell wall | | CAETHG_2613 | * | * |
| | bisynthesis | | | | |
| 4275 | hypothetical protein | | CAETHG_2614 | * | * |
| 4276 | glycosyltransferase | | CAETHG_2624 | * | * |
| 4277 | hypothetical protein | | CAETHG_2648 | * | * |
| 4278 | hypothetical protein | | CAETHG_2665 | * | * |
| 4279 | hypothetical protein | | CAETHG_2676 | * | * |
| 4280 | hypothetical protein | | CAETHG_2686 | * | * |
| 4281 | Helix-turn-helix domain-containing protein | | CAETHG_2715 | * | * |
| 4282 | hypothetical protein | | CAETHG_2736 | * | * |
| 4283 | argininosuccinate synthase | | CAETHG_2760 | * | * |
| 4284 | hypothetical protein | | CAETHG_2804 | * | * |
| 4285 | selenocysteine-specific elongation factor | | CAETHG_2841 | * | * |
| 4286 | Four helix bundle sensory module for signal transduction | | CAETHG_2857 | * | * |
| 4287 | 16S rRNA (uracill498-N3)-methyltransferase | | CAETHG_2895 | * | * |
| 4288 | arginine:ornithine antiporter/lysine permease | | CAETHG_3023 | * | * |
| 4289 | flaG, flagellar protein | | CAETHG_3050 | * | * |
| 4290 | Glycosyl transferase family 2 | | CAETHG_3066 | * | * |
| 4291 | AAA-like domain-containing protein | | CAETHG_3080 | * | * |
| 4292 | hypothetical protein | | CAETHG_3169 | * | * |
| 4293 | hypothetical protein | | CAETHG_3210 | * | * |
| 4294 | hypothetical protein | | CAETHG_3356 | * | * |
| 4295 | hypothetical protein | | CAETHG_3366 | * | * |
| 4296 | hypothetical protein | | CAETHG_3421 | * | * |
| 4297 | 5′-phosphate synthase pdxT subunit | | CAETHG_3434 | * | * |
| 4298 | BlaI family transcriptional regulator, penicillinase repressor | | CAETHG_3439 | * | * |
| 4299 | Protein of unknown function DUF3793 | | CAETHG_3484 | * | * |
| 4300 | Na+-driven multidrug efflux pump | | CAETHG_3501 | * | * |
| 4301 | Uncharacterized conserved protein | | CAETHG_3517 | * | * |
| 4302 | hypothetical protein | | CAETHG_3518 | * | * |
| 4303 | hypothetical protein | | CAETHG_3523 | * | * |
| 4304 | chromosome partitioning protein | | CAETHG_3524 | * | * |
| 4305 | hypothetical protein | | CAETHG_3527 | * | * |
| 4306 | Uncharacterized conserved protein | | CAETHG_3529 | * | * |
| 4307 | hypothetical protein | | CAETHG_3530 | * | * |
| 4308 | His-Xaa-Ser system protein HxsD | | CAETHG_3531 | * | * |
| 4309 | His-Xaa-Ser system protein HxsD | | CAETHG_3532 | * | * |
| 4310 | hypothetical protein | | CAETHG_3533 | * | * |
| 4311 | Type IV leader peptidase family protein | | CAETHG_3534 | * | * |
| 4312 | hypothetical protein | | CAETHG_3535 | * | * |
| 4313 | His-Xaa-Ser system radical SAM maturase HxsC | | CAETHG_3536 | * | * |
| 4314 | His-Xaa-Ser system radical SAM maturase HxsB | | CAETHG_3537 | * | * |
| 4315 | hypothetical protein | | CAETHG_3539 | * | * |
| 4316 | hypothetical protein | | CAETHG_3540 | * | * |
| 4317 | radical SAM additional 4Fe4S-binding SPASM domain-containing | | CAETHG_3541 | * | * |
| | protein | | | | |
| 4318 | Resolvase, N terminal domain | | CAETHG_3550 | * | * |
| 4319 | Phage-related protein | | CAETHG_3551 | * | * |
| 4320 | Helix-turn-helix | | CAETHG_3552 | * | * |
| 4321 | Diadenosine tetraphosphate (Ap4A) hydrolase | | CAETHG_3556 | * | * |
| 4322 | ATP-dependent helicase IRC3 | | CAETHG_3557 | * | * |
| 4323 | Predicted house-cleaning noncanonical NTP pyrophosphatase, all- | | CAETHG_3558 | * | * |
| | alpha NTP-PPase (MazG) superfamily | | | | |
| 4324 | AAA domain (dynein-related subfamily) | | CAETHG_3559 | * | * |
| 4325 | hypothetical protein | | CAETHG_3560 | * | * |
| 4326 | hypothetical protein | | CAETHG_3561 | * | * |
| 4327 | Immunity protein Imm6 | | CAETHG_3562 | * | * |
| 4328 | Helix-turn-helix domain-containing protein | | CAETHG_3567 | * | * |
| 4329 | Putative cell wall-binding protein | | CAETHG_3577 | * | * |
| 4330 | transcriptional regulator, IclR family | | CAETHG_3583 | * | * |
| 4331 | hydroxymethylglutaryl-CoA synthase | | CAETHG_3584 | * | * |
| 4332 | acetyl-CoA C-acetyltransferase | | CAETHG_3585 | * | * |
| 4333 | hypothetical protein | | CAETHG_3586 | * | * |
| 4334 | Predicted arabinose efflux permease, MFS family | | CAETHG_3587 | * | * |
| 4335 | Predicted arabinose efflux permease, MFS family | | CAETHG_3588 | * | * |
| 4336 | hypothetical protein | | CAETHG_3589 | * | * |
| 4337 | hypothetical protein | | CAETHG_3599 | * | * |
| 4338 | hypothetical protein | | CAETHG_3667 | * | * |
| 4339 | hypothetical protein | | CAETHG_3687 | * | * |
| 4340 | hypothetical protein | | CAETHG_3688 | * | * |
| 4341 | tryptophan synthase, alpha chain | | CAETHG_3708 | * | * |
| 4342 | Spore germination protein | | CAETHG_3745 | * | * |
| 4343 | hypothetical protein | | CAETHG_3756 | * | * |
| 4344 | hypothetical protein | | CAETHG_3758 | * | * |
| 4345 | hypothetical protein | | CAETHG_3759 | * | * |
| 4346 | IseA DL-endopeptidase inhibitor | | CAETHG_3760 | * | * |
| 4347 | putative transcriptional regulator | | CAETHG_3761 | * | * |
| 4348 | hypothetical protein | | CAETHG_3762 | * | * |
| 4349 | hypothetical protein | | CAETHG_3764 | * | * |
| 4350 | hypothetical protein | | CAETHG_3765 | * | * |
| 4351 | hypothetical protein | | CAETHG_3766 | * | * |
| 4352 | RNA-dependent RNA polymerase | | CAETHG_3767 | * | * |
| 4353 | hypothetical protein | | CAETHG_3768 | * | * |
| 4354 | Excinuclease ABC C subunit domain protein | | CAETHG_3769 | * | * |
| 4355 | hypothetical protein | | CAETHG_3770 | * | * |
| 4356 | Protein of unknown function (DUF4236) | | CAETHG_3771 | * | * |
| 4357 | hypothetical protein | | CAETHG_3772 | * | * |
| 4358 | hypothetical protein | | CAETHG_3773, | * | * |
| | | | CAETHG_3801 | | |
| 4359 | hypothetical protein | | CAETHG_3774 | * | * |
| 4360 | hypothetical protein | | CAETHG_3775 | * | * |
| 4361 | hypothetical protein | | CAETHG_3776 | * | * |
| 4362 | phage terminase, small subunit, putative, P27 | | CAETHG_3777 | * | * |
| | family | | | | |
| 4363 | Phage terminase-like protein, large subunit, contains N-terminal | | CAETHG_3778 | * | * |
| | HTH domain | | | | |
| 4364 | hypothetical protein | | CAETHG_3779 | * | * |
| 4365 | phage portal protein, HK97 family | | CAETHG_3780 | * | * |
| 4366 | ATP-dependent protease ClpP, protease subunit | | CAETHG_3781 | * | * |
| 4367 | phage major capsid protein, HK97 family | | CAETHG_3782 | * | * |
| 4368 | uncharacterized phage protein (possible DNA packaging) | | CAETHG_3783 | * | * |
| 4369 | Phage head-tail joining protein | | CAETHG_3784 | * | * |
| 4370 | Bacteriophage HK97-gp10, putative tail- | | CAETHG_3785 | * | * |
| | component | | | | |
| 4371 | hypothetical protein | | CAETHG_3786 | * | * |
| 4372 | Phage tail sheath protein | | CAETHG_3787 | * | * |
| 4373 | Phage tail tube protein | | CAETHG_3788 | * | * |
| 4374 | hypothetical protein | | CAETHG_3789 | * | * |
| 4375 | SLT domain-containing protein | | CAETHG_3790 | * | * |
| 4376 | hypothetical protein | | CAETHG_3791 | * | * |
| 4377 | NlpC/P60 family protein | | CAETHG_3792 | * | * |
| 4378 | Protein of unknown function (DUF2577) | | CAETHG_3793 | * | * |
| 4379 | Protein of unknown function (DUF2634) | | CAETHG_3794 | * | * |
| 4380 | Uncharacterized phage protein gp47/JayE | | CAETHG_3795 | * | * |
| 4381 | hypothetical protein (DUF2313) | | CAETHG_3796 | * | * |
| 4382 | parallel beta-helix repeat (two copies) | | CAETHG_3797 | * | * |
| 4383 | Haemolysin XhlA | | CAETHG_3798 | * | * |
| 4384 | Lyzozyme M1 (1,4-beta-N-acetylmuramidase), GH25 family | | CAETHG_3799 | * | * |
| 4385 | hypothetical protein | | CAETHG_3800 | * | * |
| 4386 | PemK-like, MazF-like toxin of type II toxin-antitoxin system | | CAETHG_3802 | * | * |
| 4387 | hypothetical protein | | CAETHG_3811 | * | * |
| 4388 | extracellular solute-binding protein | | CAETHG_3832 | * | * |
| 4389 | hypothetical protein | | CAETHG_3902 | * | * |
| 4390 | hypothetical protein | | CAETHG_3973 | * | * |
| 4391 | hypothetical protein | | CAETHG_3974 | * | * |
| 4392 | Broad-specificity NMP kinase | | CAETHG_3976 | * | * |
| 4393 | Methyltransferase domain-containing protein | | CAETHG_3977 | * | * |
| 4394 | protein of unknown function (DUF4351) | | CAETHG_3978 | * | * |
| 4395 | hypothetical protein | | CAETHG_3986 | * | * |
| 4396 | Collagen triple helix repeat-containing protein | | CAETHG_4029 | * | * |
| 4397 | hypothetical protein | | CAETHG_4052 | * | * |
| 4398 | hypothetical protein | | * | * | CLRAG_00050 |
| 4399 | multidrug resistance protein MdtC | | * | * | CLRAG_00060 |
| 4400 | multidrug resistance protein MdtA precursor | | * | * | CLRAG_00070 |
| 4401 | fatty acid metabolism regulator protein | | * | * | CLRAG_00080 |
| 4402 | right origin-binding protein | | * | * | CLRAG_00140 |
| 4403 | hypothetical protein | | * | * | CLRAG_00150 |
| 4404 | hypothetical protein | | * | * | CLRAG_00160 |
| 4405 | hypothetical protein | | * | * | CLRAG_00170 |
| 4406 | xylulose kinase | | * | * | CLRAG_00360 |
| 4407 | nickel-binding periplasmic protein precursor | | * | * | CLRAG_00400 |
| 4408 | glutathione import ATP-binding protein GsiA | | * | * | CLRAG_00410 |
| 4409 | oligopeptide transport ATP-binding protein OppD | | * | * | CLRAG_00420 |
| 4410 | glutathione transport system permease protein | | * | * | CLRAG_00430 |
| | GsiD | | | | |
| 4411 | glutathione transport system permease protein | | * | * | CLRAG_00440 |
| | GsiC | | | | |
| 4412 | nickel transport system permease protein NikB | | * | * | CLRAG_00450 |
| 4413 | putative methyltransferase YcgJ | | * | * | CLRAG_00460 |
| 4414 | serine/threonine transporter SstT | | * | * | CLRAG_00740 |
| 4415 | oxygen regulatory protein NreC | | * | * | CLRAG_00750 |
| 4416 | putative oxidoreductase YdhV | | * | * | CLRAG_00770 |
| 4417 | cell division inhibitor MinD | | * | * | CLRAG_00810 |
| 4418 | hypothetical protein | | * | * | CLRAG_00820 |
| 4419 | hypothetical protein | | * | * | CLRAG_00830 |
| 4420 | hypothetical protein | | * | * | CLRAG_00840 |
| 4421 | hypothetical protein | | * | * | CLRAG_00850 |
| 4422 | putative ABC transporter ATP-binding protein YxlF | | * | * | CLRAG_00860 |
| 4423 | hypothetical protein | | * | * | CLRAG_01050 |
| 4424 | demethylrebeccamycin-D-glucose O-methyltransferase | | * | * | CLRAG_01080 |
| 4425 | hypothetical protein | | * | * | CLRAG_01160 |
| 4426 | putative electron transport protein YccM | | * | * | CLRAG_01220 |
| 4427 | ribonuclease D | | * | * | CLRAG_01280 |
| 4428 | HTH-type transcriptional regulatory protein GabR | | * | * | CLRAG_01380 |
| 4429 | hypothetical protein | | * | * | CLRAG_01560 |
| 4430 | CRISPR associated protein Cas6 | | * | * | CLRAG_01570 |
| 4431 | CRISPR-associated protein (cas_TM1802) | | * | * | CLRAG_01580 |
| 4432 | hypothetical protein | | * | * | CLRAG_01590 |
| 4433 | CRISPR-associated protein (Cas_Cas5) | | * | * | CLRAG_01600 |
| 4434 | CRISPR-associated nuclease/helicase Cas3 | | * | * | CLRAG_01610 |
| 4435 | hypothetical protein | | * | * | CLRAG_01620 |
| 4436 | CRISPR-associated endonuclease Cas1 | | * | * | CLRAG_01630 |
| 4437 | CRISPR-associated endoribonuclease Cas2 | | * | * | CLRAG_01640 |
| 4438 | hypothetical protein | | * | * | CLRAG_01830 |
| 4439 | uroporphyrinogen decarboxylase | | * | * | CLRAG_02060 |
| 4440 | magnesium-chelatase 38 kDa subunit | | * | * | CLRAG_02080 |
| 4441 | magnesium-chelatase 38 kDa subunit | | * | * | CLRAG_02090 |
| 4442 | aerobic cobaltochelatase subunit CobN | | * | * | CLRAG_02100 |
| 4443 | N-acetylmuramoyl-L-alanine amidase LytC | | * | * | CLRAG_02110 |
| | precursor | | | | |
| 4444 | hemin transport system permease protein HmuU | | * | * | CLRAG_02120 |
| 4445 | putative siderophore transport system ATP-binding protein YusV | | * | * | CLRAG_02130 |
| 4446 | vitamin B12-binding protein precursor | | * | * | CLRAG_02140 |
| 4447 | hypothetical protein | | * | * | CLRAG_02230 |
| 4448 | YqaJ-like viral recombinase domain protein | | * | * | CLRAG_02240 |
| 4449 | hypothetical protein | | * | * | CLRAG_02250 |
| 4450 | HTH-type transcriptional regulator ImmR | | * | * | CLRAG_02260 |
| 4451 | hypothetical protein | | * | * | CLRAG_02270 |
| 4452 | hypothetical protein | | * | * | CLRAG_02300 |
| 4453 | hypothetical protein | | * | * | CLRAG_02310 |
| 4454 | GTP pyrophosphokinase YjbM | | * | * | CLRAG_02320 |
| 4455 | hypothetical protein | | * | * | CLRAG_02330 |
| 4456 | 50S ribosomal protein L22/unknown domain fusion protein | | * | * | CLRAG_02340 |
| 4457 | hypothetical protein | | * | * | CLRAG_02350 |
| 4458 | hypothetical protein | | * | * | CLRAG_02360 |
| 4459 | hypothetical protein | | * | * | CLRAG_02370 |
| 4460 | hypothetical protein | | * | * | CLRAG_02380 |
| 4461 | hypothetical protein | | * | * | CLRAG_02390 |
| 4462 | propanediol utilization protein PduA | | * | * | CLRAG_02940 |
| 4463 | biotin transporter BioY2 | | * | * | CLRAG_03060 |
| 4464 | biotin synthase | | * | * | CLRAG_03070 |
| 4465 | cocaine esterase | | * | * | CLRAG_03080 |
| 4466 | HTH-type transcriptional repressor Bm3R1 | | * | * | CLRAG_03090 |
| 4467 | hypothetical protein | | * | * | CLRAG_03100 |
| 4468 | hypothetical protein | | * | * | CLRAG_03110 |
| 4469 | hypothetical protein | | * | * | CLRAG_03150 |
| 4470 | homoserine/homoserine lactone efflux protein | | * | * | CLRAG_03160 |
| 4471 | HTH-type transcriptional regulatory protein GabR | | * | * | CLRAG_03170 |
| 4472 | putative sensory transducer protein YfmS | | * | * | CLRAG_03180 |
| 4473 | ferredoxin | | * | * | CLRAG_03190 |
| 4474 | benzylsuccinate synthase alpha subunit | | * | * | CLRAG_03200 |
| 4475 | hypothetical protein | | * | * | CLRAG_03210 |
| 4476 | hypothetical protein | | * | * | CLRAG_03220 |
| 4477 | hypothetical protein | | * | * | CLRAG_03230 |
| 4478 | hypothetical protein | | * | * | CLRAG_03260 |
| 4479 | putative peptidoglycan binding domain protein | | * | * | CLRAG_03270 |
| 4480 | galactoside O-acetyltransferase | | * | * | CLRAG_03280 |
| 4481 | putative ribosomal N-acetyltransferase YdaF | | * | * | CLRAG_03290 |
| 4482 | regulatory protein SoxS | | * | * | CLRAG_03300 |
| 4483 | hypothetical protein | | * | * | CLRAG_03320 |
| 4484 | helix-turn-helix | | * | * | CLRAG_03380 |
| 4485 | hypothetical protein | | * | * | CLRAG_03410 |
| 4486 | hypothetical protein | | * | * | CLRAG_03420 |
| 4487 | hypothetical protein | | * | * | CLRAG_03430 |
| 4488 | hypothetical protein | | * | * | CLRAG_03440 |
| 4489 | CAAX amino terminal protease self- immunity | | * | * | CLRAG_03800 |
| 4490 | putative ribosomal N-acetyltransferase YdaF | | * | * | CLRAG_03860 |
| 4491 | hypothetical protein | | * | * | CLRAG_03940 |
| 4492 | DNA-binding transcriptional repressor MarR | | * | * | CLRAG_03950 |
| 4493 | flavodoxin | | * | * | CLRAG_03960 |
| 4494 | sensor protein kinase WalK | | * | * | CLRAG_04310 |
| 4495 | transcriptional regulatory protein WalR | | * | * | CLRAG_04320 |
| 4496 | TVP38/TMEM64 family inner membrane protein | | * | * | CLRAG_04330 |
| | YdjZ | | | | |
| 4497 | phosphatidylcholine synthase | | * | * | CLRAG_04340 |
| 4498 | Ktr system potassium uptake protein A | | * | * | CLRAG_04460 |
| 4499 | Ktr system potassium uptake protein B | | * | * | CLRAG_04470 |
| 4500 | putative sensory transducer protein YfmS | | * | * | CLRAG_04500 |
| 4501 | N-acetylmuramoyl-L-alanine amidase LytC | | * | * | CLRAG_05510 |
| | precursor | | | | |
| 4502 | hypothetical protein | | * | * | CLRAG_05520 |
| 4503 | methyl-accepting chemotaxis protein McpA | | * | * | CLRAG_05560 |
| 4504 | nitrogenase molybdenum-iron protein alpha chain | | * | * | CLRAG_05590 |
| 4505 | nitrogenase molybdenum-iron protein beta chain | | * | * | CLRAG_05600 |
| 4506 | FeMo cofactor biosynthesis protein NifB | | * | * | CLRAG_05620 |
| 4507 | methyl-accepting chemotaxis protein 4 | | * | * | CLRAG_05770 |
| 4508 | FIST N domain protein | | * | * | CLRAG_05780 |
| 4509 | hypothetical protein | | * | * | CLRAG_05830 |
| 4510 | 6-aminohexanoate-dimer hydrolase | | * | * | CLRAG_05890 |
| 4511 | UvrABC system protein A | | * | * | CLRAG_05900 |
| 4512 | HTH-type transcriptional activator Btr | | * | * | CLRAG_05910 |
| 4513 | carbon starvation protein A | | * | * | CLRAG_06580 |
| 4514 | oligopeptide transport ATP-binding protein OppD | | * | * | CLRAG_06890 |
| 4515 | epoxyqueuosine reductase | | * | * | CLRAG_06980 |
| 4516 | hypothetical protein | | * | * | CLRAG_07010 |
| 4517 | GDSL-like lipase/acylhydrolase | | * | * | CLRAG_07020 |
| 4518 | 3′,5′-cyclic adenosine monophosphate phosphodiesterase CpdA | | * | * | CLRAG_07200 |
| 4519 | chromosome segregation protein | | * | * | CLRAG_07280 |
| 4520 | internalin-A precursor | | * | * | CLRAG_07290 |
| 4521 | internalin-A precursor | | * | * | CLRAG_07300 |
| 4522 | hypothetical protein | | * | * | CLRAG_07360 |
| 4523 | HTH-type transcriptional regulator CynR | | * | * | CLRAG_08110 |
| 4524 | hypothetical protein | | * | * | CLRAG_08690 |
| 4525 | alkaline phosphatase synthesis transcriptional regulatory protein | | * | * | CLRAG_08810 |
| | PhoP | | | | |
| 4526 | hypothetical protein | | * | * | CLRAG_08840 |
| 4527 | hypothetical protein | | * | * | CLRAG_08850 |
| 4528 | hypothetical protein | | * | * | CLRAG_08860 |
| 4529 | type-1 restriction enzyme R protein | | * | * | CLRAG_08890 |
| 4530 | type I restriction modification DNA specificity domain protein | | * | * | CLRAG_08900 |
| 4531 | putative type I restriction enzymeP M protein | | * | * | CLRAG_08910 |
| 4532 | glutamate racemase | | * | * | CLRAG_08930 |
| 4533 | ferredoxin | | * | * | CLRAG_08950 |
| 4534 | methyl-accepting chemotaxis protein McpC | | * | * | CLRAG_08990 |
| 4535 | N-substituted formamide deformylase precursor | | * | * | CLRAG_09020 |
| 4536 | multidrug export protein MepA | | * | * | CLRAG_09030 |
| 4537 | spore germination protein GerE | | * | * | CLRAG_09050 |
| 4538 | carboxylesterase NlhH | | * | * | CLRAG_09110 |
| 4539 | hypothetical protein | | * | * | CLRAG_09120 |
| 4540 | hypothetical protein | | * | * | CLRAG_09200 |
| 4541 | multiple antibiotic resistance protein MarA | | * | * | CLRAG_09210 |
| 4542 | cyclic di-GMP phosphodiesterase response regulator RpfG | | * | * | CLRAG_09300 |
| 4543 | peroxide-responsive repressor PerR | | * | * | CLRAG_09310 |
| 4544 | putative sensory transducer protein YfmS | | * | * | CLRAG_09350 |
| 4545 | copper-sensing transcriptional repressor CsoR | | * | * | CLRAG_09360 |
| 4546 | putative copper-exporting P-type ATPase V | | * | * | CLRAG_09370 |
| 4547 | DNA-binding transcriptional regulator AraC | | * | * | CLRAG_09380 |
| 4548 | alpha/beta hydrolase family protein | | * | * | CLRAG_09390 |
| 4549 | right origin-binding protein | | * | * | CLRAG_09400 |
| 4550 | demethylrebeccamycin-D-glucose O-methyltransferase | | * | * | CLRAG_09410 |
| 4551 | iron import ATP-binding/permease protein IrtA | | * | * | CLRAG_09420 |
| 4552 | putative multidrug export ATP-binding/permease protein | | * | * | CLRAG_09430 |
| 4553 | hypothetical protein | | * | * | CLRAG_09490 |
| 4554 | hypothetical protein | | * | * | CLRAG_09610 |
| 4555 | hypothetical protein | | * | * | CLRAG_09620 |
| 4556 | hypothetical protein | | * | * | CLRAG_09630 |
| 4557 | chromosome partition protein Smc | | * | * | CLRAG_09640 |
| 4558 | hypothetical protein | | * | * | CLRAG_09700 |
| 4559 | phosphoadenosine phosphosulfate reductase | | * | * | CLRAG_09760 |
| 4560 | hypothetical protein | | * | * | CLRAG_09770 |
| 4561 | hypothetical protein | | * | * | CLRAG_09780 |
| 4562 | hypothetical protein | | * | * | CLRAG_09790 |
| 4563 | cysteine desulfurase SufS | | * | * | CLRAG_09800 |
| 4564 | type III restriction enzyme, res subunit | | * | * | CLRAG_09810 |
| 4565 | hypothetical protein | | * | * | CLRAG_09820 |
| 4566 | chromosome partition protein Smc | | * | * | CLRAG_09830 |
| 4567 | hypothetical protein | | * | * | CLRAG_09840 |
| 4568 | RNA polymerase sigma factor RpoS | | * | * | CLRAG_09850 |
| 4569 | endonuclease/exonuclease/phosphatase family protein | | * | * | CLRAG_09880 |
| 4570 | hypothetical protein | | * | * | CLRAG_09890 |
| 4571 | plasmid pRiA4b ORF-3-like protein | | * | * | CLRAG_09900 |
| 4572 | hypothetical protein | | * | * | CLRAG_09930 |
| 4573 | hydroperoxy fatty acid reductase gpx1 | | * | * | CLRAG_10140 |
| 4574 | organic hydroperoxide resistance transcriptional regulator | | * | * | CLRAG_10150 |
| 4575 | alanine--tRNA ligase | | * | * | CLRAG_10160 |
| 4576 | hypothetical protein | | * | * | CLRAG_10960 |
| 4577 | hypothetical protein | | * | * | CLRAG_11060 |
| 4578 | replication-associated recombination protein A | | * | * | CLRAG_11710 |
| 4579 | putative multidrug resistance protein EmrK | | * | * | CLRAG_12840 |
| 4580 | hypothetical protein | | * | * | CLRAG_13370 |
| 4581 | hypothetical protein | | * | * | CLRAG_13390 |
| 4582 | hypothetical protein | | * | * | CLRAG_13400 |
| 4583 | hypothetical protein | | * | * | CLRAG_13420 |
| 4584 | SPBc2 prophage-derived glycosyltransferase SunS | | * | * | CLRAG_13430 |
| 4585 | hypothetical protein | | * | * | CLRAG_13440 |
| 4586 | hypothetical protein | | * | * | CLRAG_13450 |
| 4587 | flagellin | | * | * | CLRAG_13460 |
| 4588 | hypothetical protein | | * | * | CLRAG_13490 |
| 4589 | flagellar hook-associated protein FlgL | | * | * | CLRAG_13580 |
| 4590 | N-acetylmuramoyl-L-alanine amidase LytC | | * | * | CLRAG_13940 |
| | precursor | | | | |
| 4591 | ABC transporter permease protein YxdM | | * | * | CLRAG_14390 |
| 4592 | sensor histidine kinase GraS | | * | * | CLRAG_14410 |
| 4593 | glyoxylate/hydroxypyruvate reductase A | | * | * | CLRAG_14820 |
| 4594 | sugar phosphatase YidA | | * | * | CLRAG_14920 |
| 4595 | phosphomethylpyrimidine synthase | | * | * | CLRAG_15080 |
| 4596 | amidophosphoribosyltransferase precursor | | * | * | CLRAG_15090 |
| 4597 | amidophosphoribosyltransferase precursor | | * | * | CLRAG_15100 |
| 4598 | hypothetical protein | | * | * | CLRAG_15150 |
| 4599 | isoprenyl transferase | | * | * | CLRAG_15210 |
| 4600 | putative sugar kinase YdjH | | * | * | CLRAG_15530 |
| 4601 | permease for cytosine/purines, uracil, thiamine, allantoin | | * | * | CLRAG_15540 |
| 4602 | ADP-ribosylglycohydrolase | | * | * | CLRAG_15550 |
| 4603 | putative HTH-type transcriptional regulator YurK | | * | * | CLRAG_15560 |
| 4604 | ATP-binding region | | * | * | CLRAG_15700 |
| 4605 | hypothetical protein | | * | * | CLRAG_15750 |
| 4606 | reactivating factor for ethanolamine ammonia | | * | * | CLRAG_15830 |
| | lyase | | | | |
| 4607 | formamidase | | * | * | CLRAG_15860 |
| 4608 | Low-affinity putrescine importer PlaP | | * | * | CLRAG_15870 |
| 4609 | hypothetical protein | | * | * | CLRAG_15890 |
| 4610 | acetoacetate decarboxylase (ADC) | | * | * | CLRAG_15900 |
| 4611 | putative diguanylate cyclase YedQ | | * | * | CLRAG_15960 |
| 4612 | putative thiazole biosynthetic enzyme | | * | * | CLRAG_15990 |
| 4613 | AB hydrolase superfamily protein YdjP | | * | * | CLRAG_16030 |
| 4614 | putrescine importer PuuP | | * | * | CLRAG_16040 |
| 4615 | hypothetical protein | | * | * | CLRAG_16090 |
| 4616 | chagasin family peptidase inhibitor I42 | | * | * | CLRAG_16320 |
| 4617 | hypothetical protein | | * | * | CLRAG_16330 |
| 4618 | hypothetical protein | | * | * | CLRAG_16340 |
| 4619 | hypothetical protein | | * | * | CLRAG_16360 |
| 4620 | putative glycosyl transferase | | * | * | CLRAG_16400 |
| 4621 | methyl-accepting chemotaxis protein 4 | | * | * | CLRAG_16630 |
| 4622 | outer membrane protein assembly factor BamE | | * | * | CLRAG_16660 |
| 4623 | hypothetical protein | | * | * | CLRAG_16690 |
| 4624 | hypothetical protein | | * | * | CLRAG_16700 |
| 4625 | hypothetical protein | | * | * | CLRAG_16710 |
| 4626 | hypothetical protein | | * | * | CLRAG_16720 |
| 4627 | hypothetical protein | | * | * | CLRAG_16730 |
| 4628 | tyrosine recombinase XerD | | * | * | CLRAG_16760 |
| 4629 | tyrosine recombinase XerC | | * | * | CLRAG_16770 |
| 4630 | tyrosine recombinase XerD | | * | * | CLRAG_16780 |
| 4631 | chaperone protein DnaJ | | * | * | CLRAG_16810 |
| 4632 | tetratricopeptide repeat protein | | * | * | CLRAG_16820 |
| 4633 | chaperone protein DnaK | | * | * | CLRAG_16830 |
| 4634 | protein GrpE | | * | * | CLRAG_16840 |
| 4635 | hypothetical protein | | * | * | CLRAG_16850 |
| 4636 | hypothetical protein | | * | * | CLRAG_16860 |
| 4637 | hypothetical protein | | * | * | CLRAG_16870 |
| 4638 | hypothetical protein | | * | * | CLRAG_16890 |
| 4639 | hypothetical protein | | * | * | CLRAG_16900 |
| 4640 | hypothetical protein | | * | * | CLRAG_16910 |
| 4641 | GTPase Der | | * | * | CLRAG_16920 |
| 4642 | hypothetical protein | | * | * | CLRAG_16930 |
| 4643 | putative diguanylate cyclase YdaM | | * | * | CLRAG_16940 |
| 4644 | putative diguanylate cyclase YdaM | | * | * | CLRAG_16950 |
| 4645 | multidrug resistance protein Stp | | * | * | CLRAG_16960 |
| 4646 | nuclease-related domain protein | | * | * | CLRAG_16970 |
| 4647 | hypothetical protein | | * | * | CLRAG_16990 |
| 4648 | hypothetical protein | | * | * | CLRAG_17010 |
| 4649 | hypothetical protein | | * | * | CLRAG_17020 |
| 4650 | hypothetical protein | | * | * | CLRAG_17040 |
| 4651 | hypothetical protein | | * | * | CLRAG_17050 |
| 4652 | HTH-type transcriptional activator mta | | * | * | CLRAG_17100 |
| 4653 | hypothetical protein | | * | * | CLRAG_17290 |
| 4654 | hypothetical protein | | * | * | CLRAG_17960 |
| 4655 | putative methyltransferase YcgJ | | * | * | CLRAG_18120 |
| 4656 | putative xanthine dehydrogenase subunit A | | * | * | CLRAG_18190 |
| 4657 | FmdE, molybdenum formylmethanofuran dehydrogenase operon | | * | * | CLRAG_18200 |
| 4658 | hypothetical protein | | * | * | CLRAG_18210 |
| 4659 | vitamin B12-binding protein | | * | * | CLRAG_18220 |
| 4660 | S-adenosyl-L-methionine-binding protein | | * | * | CLRAG_18230 |
| 4661 | molybdopterin molybdenumtransferase | | * | * | CLRAG_18240 |
| 4662 | molybdate transporter ATP-binding protein | | * | * | CLRAG_18250 |
| 4663 | hypothetical protein | | * | * | CLRAG_18270 |
| 4664 | acetyltransferase (GNAT) family protein | | * | * | CLRAG_18290 |
| 4665 | hypothetical protein | | * | * | CLRAG_18300 |
| 4666 | hypothetical protein | | * | * | CLRAG_18310 |
| 4667 | HTH-type transcriptional regulator GltR | | * | * | CLRAG_18330 |
| 4668 | putative NAD(P)H-dependent FMN-containing oxidoreductase | | * | * | CLRAG_18340 |
| | YwqN | | | | |
| 4669 | flavodoxin | | * | * | CLRAG_18350 |
| 4670 | hypothetical protein | | * | * | CLRAG_18360 |
| 4671 | l-deoxy-D-xylulose-5-phosphate synthase | | * | * | CLRAG_18640 |
| 4672 | 3-oxoacyl-[acyl-carrier-protein] reductase FabG | | * | * | CLRAG_18650 |
| 4673 | putative tartrate transporter | | * | * | CLRAG_18660 |
| 4674 | hypothetical protein | | * | * | CLRAG_18970 |
| 4675 | hypothetical protein | | * | * | CLRAG_19420 |
| 4676 | methionine synthase | | * | * | CLRAG_19430 |
| 4677 | hypothetical protein | | * | * | CLRAG_19450 |
| 4678 | penicillinase repressor | | * | * | CLRAG_19480 |
| 4679 | regulatory protein BlaR1 | | * | * | CLRAG_19490 |
| 4680 | isoprenylcysteine carboxyl methyltransferase (ICMT) family protein | | * | * | CLRAG_20000 |
| 4681 | hypothetical protein | | * | * | CLRAG_20110 |
| 4682 | hypothetical protein | | * | * | CLRAG_20340 |
| 4683 | hypothetical protein | | * | * | CLRAG_20360 |
| 4684 | N-acetylmuramoyl-L-alanine amidase LytC | | * | * | CLRAG_20370 |
| | precursor | | | | |
| 4685 | hypothetical protein | | * | * | CLRAG_20420 |
| 4686 | transposon Tn10 TetD protein | | * | * | CLRAG_20430 |
| 4687 | ankyrin repeats (3 copies) | | * | * | CLRAG_20500 |
| 4688 | hypothetical protein | | * | * | CLRAG_20510 |
| 4689 | putative multidrug resistance protein EmrY | | * | * | CLRAG_20520 |
| 4690 | hypothetical protein | | * | * | CLRAG_20910 |
| 4691 | CsgBAC operon transcriptional regulatory protein | | * | * | CLRAG_20920 |
| 4692 | peptidase family M28 | | * | * | CLRAG_20930 |
| 4693 | putative penicillin-binding protein PbpX | | * | * | CLRAG_20980 |
| 4694 | HTH-type transcriptional activator RhaR | | * | * | CLRAG_21010 |
| 4695 | electron transport complex subunit RsxB | | * | * | CLRAG_21020 |
| 4696 | hypothetical protein | | * | * | CLRAG_21030 |
| 4697 | hypothetical protein | | * | * | CLRAG_21080 |
| 4698 | hypothetical protein | | * | * | CLRAG_21220 |
| 4699 | spore protein YkvP | | * | * | CLRAG_21370 |
| 4700 | peptidoglycan-N-acetylmuramic acid deacetylase PdaA precursor | | * | * | CLRAG_21380 |
| 4701 | spore protein YkvP | | * | * | CLRAG_21390 |
| 4702 | hypothetical protein | | * | * | CLRAG_21410 |
| 4703 | hypothetical protein | | * | * | CLRAG_21420 |
| 4704 | putative sulfoacetate transporter SauU | | * | * | CLRAG_21570 |
| 4705 | hypothetical protein | | * | * | CLRAG_21770 |
| 4706 | triosephosphate isomerase | | * | * | CLRAG_21830 |
| 4707 | hypothetical protein | | * | * | CLRAG_21840 |
| 4708 | putative glucarate transporter | | * | * | CLRAG_21850 |
| 4709 | putative sugar phosphate isomerase YwlF | | * | * | CLRAG_21870 |
| 4710 | HTH-type transcriptional repressor GlcR | | * | * | CLRAG_21890 |
| 4711 | cyclic pyranopterin monophosphate synthase | | * | * | CLRAG_22260 |
| 4712 | 4-hydroxyphenylacetate decarboxylase large | | * | * | CLRAG_22270 |
| | subunit | | | | |
| 4713 | 4-hydroxyphenylacetate decarboxylase activating enzyme | | * | * | CLRAG_22280 |
| 4714 | methyl-accepting chemotaxis protein 3 | | * | * | CLRAG_22290 |
| 4715 | hypothetical protein | | * | * | CLRAG_22300 |
| 4716 | nuclease-related domain protein | | * | * | CLRAG_22310 |
| 4717 | divergent AAA domain protein | | * | * | CLRAG_22380 |
| 4718 | HTH-type transcriptional regulator AcrR | | * | * | CLRAG_22390 |
| 4719 | multidrug resistance protein 3 | | * | * | CLRAG_22400 |
| 4720 | hypothetical protein | | * | * | CLRAG_22410 |
| 4721 | hypothetical protein | | * | * | CLRAG_22420 |
| 4722 | ferredoxin | | * | * | CLRAG_22430 |
| 4723 | hypothetical protein | | * | * | CLRAG_22460 |
| 4724 | hypothetical protein | | * | * | CLRAG_22470 |
| 4725 | hypothetical protein | | * | * | CLRAG_22480 |
| 4726 | hypothetical protein | | * | * | CLRAG_22490 |
| 4727 | hypothetical protein | | * | * | CLRAG_22500 |
| 4728 | hypothetical protein | | * | * | CLRAG_22510 |
| 4729 | modification methylase HaeIII | | * | * | CLRAG_22520 |
| 4730 | hypothetical protein | | * | * | CLRAG_22530 |
| 4731 | hypothetical protein | | * | * | CLRAG_22540 |
| 4732 | sensor protein FixL | | * | * | CLRAG_22550 |
| 4733 | hypothetical protein | | * | * | CLRAG_22560 |
| 4734 | hypothetical protein | | * | * | CLRAG_22570 |
| 4735 | transposon Tn7 transposition protein TnsC | | * | * | CLRAG_22580 |
| 4736 | transposon Tn7 transposition protein TnsB | | * | * | CLRAG_22590 |
| 4737 | transposon Tn7 transposition protein TnsA | | * | * | CLRAG_22600 |
| 4738 | ribosomal RNA small subunit methyltransferase A | | * | * | CLRAG_23630 |
| 4739 | hypothetical protein | | * | * | CLRAG_23650 |
| 4740 | hypothetical protein | | * | * | CLRAG_23660 |
| 4741 | hypothetical protein | | * | * | CLRAG_23670 |
| 4742 | hypothetical protein | | * | * | CLRAG_23680 |
| 4743 | hypothetical protein | | * | * | CLRAG_23740 |
| 4744 | anaerobic benzoate catabolism transcriptional regulator | | * | * | CLRAG_23750 |
| 4745 | hypothetical protein | | * | * | CLRAG_23760 |
| 4746 | hypothetical protein | | * | * | CLRAG_23770 |
| 4747 | hypothetical protein | | * | * | CLRAG_23780 |
| 4748 | HNH endonuclease | | * | * | CLRAG_23790 |
| 4749 | hypothetical protein | | * | * | CLRAG_23800 |
| 4750 | hypothetical protein | | * | * | CLRAG_23810 |
| 4751 | DNA replication and repair protein RecF | | * | * | CLRAG_23820 |
| 4752 | hypothetical protein | | * | * | CLRAG_23830 |
| 4753 | hypothetical protein | | * | * | CLRAG_23860 |
| 4754 | serine/threonine-protein kinase PrkC | | * | * | CLRAG_23870 |
| 4755 | hypothetical protein | | * | * | CLRAG_23880 |
| 4756 | galactoside O-acetyltransferase | | * | * | CLRAG_23890 |
| 4757 | hypothetical protein | | * | * | CLRAG_23900 |
| 4758 | Hca operon transcriptional activator | | * | * | CLRAG_23910 |
| 4759 | NADH oxidase | | * | * | CLRAG_23920 |
| 4760 | demethylmenaquinone methyltransferase | | * | * | CLRAG_23970 |
| 4761 | hypothetical protein | | * | * | CLRAG_24030 |
| 4762 | hypothetical protein | | * | * | CLRAG_24240 |
| 4763 | hypothetical protein | | * | * | CLRAG_24250 |
| 4764 | hypothetical protein | | * | * | CLRAG_24260 |
| 4765 | CAAX amino terminal protease self- immunity | | * | * | CLRAG_24400 |
| 4766 | outer membrane protein assembly factor BamD | | * | * | CLRAG_24860 |
| 4767 | putative cell wall binding repeat 2 | | * | * | CLRAG_24870 |
| 4768 | hypothetical protein | | * | * | CLRAG_25030 |
| 4769 | altronate dehydratase | | * | * | CLRAG_25040 |
| 4770 | 2-keto-3-deoxygluconate permease | | * | * | CLRAG_25050 |
| 4771 | 2-dehydro-3-deoxygluconokinase | | * | * | CLRAG_25060 |
| 4772 | transcriptional regulator KdgR | | * | * | CLRAG_25080 |
| 4773 | methyl-accepting chemotaxis protein 4 | | * | * | CLRAG_25200 |
| 4774 | ammonium transporter NrgA | | * | * | CLRAG_25480 |
| 4775 | ribosomal-protein-S5-alanine N-acetyltransferase | | * | * | CLRAG_26200 |
| 4776 | acetyltransferase (GNAT) family protein | | * | * | CLRAG_26210 |
| 4777 | hypothetical protein | | * | * | CLRAG_26230 |
| 4778 | multidrug-efflux transporter 1 regulator | | * | * | CLRAG_26240 |
| 4779 | putative protease YdeA | | * | * | CLRAG_26400 |
| 4780 | N-acetylmuramoyl-L-alanine amidase LytC | | * | * | CLRAG_26410 |
| | precursor | | | | |
| 4781 | putative transposase | | * | * | CLRAG_26740 |
| 4782 | hypothetical protein | | * | * | CLRAG_26800 |
| 4783 | tyrosine recombinase XerC | | * | * | CLRAG_26810 |
| 4784 | tyrosine recombinase XerD | | * | * | CLRAG_26820 |
| 4785 | hypothetical protein | | * | * | CLRAG_27210 |
| 4786 | hypothetical protein | | * | * | CLRAG_27610 |
| 4787 | diacylglycerol kinase | | * | * | CLRAG_28550 |
| 4788 | sodium/glucose cotransporter | | * | * | CLRAG_28930 |
| 4789 | 3-oxoacyl-[acyl-carrier-protein] reductase FabG | | * | * | CLRAG_28940 |
| 4790 | BNR/Asp-box repeat protein | | * | * | CLRAG_28950 |
| 4791 | toxin-antitoxin biofilm protein TabA | | * | * | CLRAG_28960 |
| 4792 | inner membrane transport protein YajR | | * | * | CLRAG_28970 |
| 4793 | N-acetylmuramoyl-L-alanine amidase LytC | | * | * | CLRAG_28990 |
| | precursor | | | | |
| 4794 | hypothetical protein | | * | * | CLRAG_29010 |
| 4795 | staygreen protein | | * | * | CLRAG_29020 |
| 4796 | methyl-accepting chemotaxis protein McpC | | * | * | CLRAG_29070 |
| 4797 | nitrogen regulatory protein P-II | | * | * | CLRAG_29090 |
| 4798 | hypothetical protein | | * | * | CLRAG_29100 |
| 4799 | ammonia channel precursor | | * | * | CLRAG_29110 |
| 4800 | chemotaxis protein CheY | | * | * | CLRAG_29140 |
| 4801 | CheY-P phosphatase CheC | | * | * | CLRAG_29150 |
| 4802 | cyclic di-GMP phosphodiesterase Gmr | | * | * | CLRAG_29160 |
| 4803 | hypothetical protein | | * | * | CLRAG_29340 |
| 4804 | hypothetical protein | | * | * | CLRAG_29390 |
| 4805 | chaperone protein CIpB | | * | * | CLRAG_29400 |
| 4806 | hypothetical protein | | * | * | CLRAG_29410 |
| 4807 | hypothetical protein | | * | * | CLRAG_29420 |
| 4808 | hypothetical protein | | * | * | CLRAG_29430 |
| 4809 | hypothetical protein | | * | * | CLRAG_29440 |
| 4810 | transcriptional regulatory protein ZraR | | * | * | CLRAG_29480 |
| 4811 | YvrJ protein family protein | | * | * | CLRAG_29810 |
| 4812 | hypothetical protein | | * | * | CLRAG_29880 |
| 4813 | hypothetical protein | | * | * | CLRAG_29900 |
| 4814 | hypothetical protein | | * | * | CLRAG_29910 |
| 4815 | hypothetical protein | | * | * | CLRAG_29920 |
| 4816 | chaperone protein CIpB | | * | * | CLRAG_29930 |
| 4817 | hypothetical protein | | * | * | CLRAG_29940 |
| 4818 | ubiquinone/menaquinone biosynthesis C-methyltransferase UbiE | | * | * | CLRAG_29950 |
| 4819 | heme response regulator HssR | | * | * | CLRAG_29960 |
| 4820 | alkaline phosphatase synthesis sensor protein | | * | * | CLRAG_29970 |
| | PhoR | | | | |
| 4821 | purine efflux pump PbuE | | * | * | CLRAG_29980 |
| 4822 | transposase | | * | * | CLRAG_30020 |
| 4823 | N-acyl homoserine lactonase | | * | * | CLRAG_30030 |
| 4824 | aldehyde oxidoreductase | | * | * | CLRAG_30550 |
| 4825 | Na(+)-translocating NADH-quinone reductase subunit F | | * | * | CLRAG_30670 |
| 4826 | hypothetical protein | | * | * | CLRAG_30720 |
| 4827 | inner membrane protein YdgC | | * | * | CLRAG_30750 |
| 4828 | ribosomal small subunit pseudouridine synthase A | | * | * | CLRAG_31120 |
| 4829 | hypothetical protein | | * | * | CLRAG_31130 |
| 4830 | LysM domain protein | | * | * | CLRAG_31150 |
| 4831 | gluconate 5-dehydrogenase | | * | * | CLRAG_31660 |
| 4832 | chemotaxis response regulator protein-glutamate methylesterase | | * | * | CLRAG_31670 |
| 4833 | hypothetical protein | | * | * | CLRAG_31950 |
| 4834 | bacterial Ig-like domain (group 2) | | * | * | CLRAG_31970 |
| 4835 | hypothetical protein | | * | * | CLRAG_31980 |
| 4836 | bacterial Ig-like domain (group 2) | | * | * | CLRAG_32030 |
| 4837 | hypothetical protein | | * | * | CLRAG_32190 |
| 4838 | hypothetical protein | | * | * | CLRAG_32360 |
| 4839 | hypothetical protein | | * | * | CLRAG_32440 |
| 4840 | hypothetical protein | | * | * | CLRAG_32450 |
| 4841 | recombination and repair protein RecT | | * | * | CLRAG_32460 |
| 4842 | hypothetical protein | | * | * | CLRAG_32490 |
| 4843 | formate dehydrogenase H | | * | * | CLRAG_32520 |
| 4844 | hypothetical protein | | * | * | CLRAG_32740 |
| 4845 | hypothetical protein | | * | * | CLRAG_32950 |
| 4846 | hypothetical protein | | * | * | CLRAG_33250 |
| 4847 | hypothetical protein | | * | * | CLRAG_33300 |
| 4848 | hypothetical protein | | * | * | CLRAG_33320 |
| 4849 | tyrosine recombinase XerC | | * | * | CLRAG_33370 |
| 4850 | hypothetical protein | | * | * | CLRAG_33380 |
| 4851 | hypothetical protein | | * | * | CLRAG_33430 |
| 4852 | hypothetical protein | | * | * | CLRAG_33450 |
| 4853 | hypothetical protein | | * | * | CLRAG_33460 |
| 4854 | helix-turn-helix | | * | * | CLRAG_33470 |
| 4855 | hypothetical protein | | * | * | CLRAG_33480 |
| 4856 | hypothetical protein | | * | * | CLRAG_33520 |
| 4857 | hypothetical protein | | * | * | CLRAG_33550 |
| 4858 | hypothetical protein | | * | * | CLRAG_33570 |
| 4859 | hypothetical protein | | * | * | CLRAG_33610 |
| 4860 | hypothetical protein | | * | * | CLRAG_33620 |
| 4861 | hypothetical protein | | * | * | CLRAG_33630 |
| 4862 | DEAD/DEAH box helicase | | * | * | CLRAG_33640 |
| 4863 | hypothetical protein | | * | * | CLRAG_33650 |
| 4864 | hypothetical protein | | * | * | CLRAG_33660 |
| 4865 | hypothetical protein | | * | * | CLRAG_33670 |
| 4866 | hypothetical protein | | * | * | CLRAG_33680 |
| 4867 | hypothetical protein | | * | * | CLRAG_33690 |
| 4868 | N-6 DNA methylase | | * | * | CLRAG_33700 |
| 4869 | hypothetical protein | | * | * | CLRAG_33710 |
| 4870 | hypothetical protein | | * | * | CLRAG_33730 |
| 4871 | hypothetical protein | | * | * | CLRAG_33740 |
| 4872 | hypothetical protein | | * | * | CLRAG_33750 |
| 4873 | hypothetical protein | | * | * | CLRAG_33760 |
| 4874 | DNA-invertase hin | | * | * | CLRAG_33770 |
| 4875 | hypothetical protein | | * | * | CLRAG_33780 |
| 4876 | hypothetical protein | | * | * | CLRAG_33790 |
| 4877 | outer membrane protein assembly factor BamD | | * | * | CLRAG_33800 |
| 4878 | tetratricopeptide repeat protein | | * | * | CLRAG_33810 |
| 4879 | hypothetical protein | | * | * | CLRAG_33830 |
| 4880 | group II intron-encoded protein LtrA | | * | * | CLRAG_33840 |
| 4881 | transposase IS200 like protein | | * | * | CLRAG_33850 |
| 4882 | hypothetical protein | | * | * | CLRAG_33920 |
| 4883 | bacterial Ig-like domain (group 2) | | * | * | CLRAG_34030 |
| 4884 | hypothetical protein | | * | * | CLRAG_34040 |
| 4885 | hypothetical protein | | * | * | CLRAG_34050 |
| 4886 | hypothetical protein | | * | * | CLRAG_34060 |
| 4887 | hypothetical protein | | * | * | CLRAG_34070 |
| 4888 | putative HMP/thiamine import ATP-binding protein YkoD | | * | * | CLRAG_34080 |
| 4889 | energy-coupling factor transporter ATP-binding protein EcfA2 | | * | * | CLRAG_34090 |
| 4890 | energy-coupling factor transporter transmembrane protein EcfT | | * | * | CLRAG_34100 |
| 4891 | oxidoreductase molybdopterin binding domain | | * | * | CLRAG_34110 |
| | protein | | | | |
| 4892 | hypothetical protein | | * | * | CLRAG_34120 |
| 4893 | cell-cell adhesion domain protein | | * | * | CLRAG_34130 |
| 4894 | kappa-carrageenase precursor | | * | * | CLRAG_34140 |
| 4895 | hypothetical protein | | * | * | CLRAG_34150 |
| 4896 | 3′,5′-cyclic adenosine monophosphate phosphodiesterase CpdA | | * | * | CLRAG_34160 |
| 4897 | hypothetical protein | | * | * | CLRAG_34170 |
| 4898 | NHL repeat protein | | * | * | CLRAG_34180 |
| 4899 | N-acetylmuramoyl-L-alanine amidase LytC | | * | * | CLRAG_34190 |
| | precursor | | | | |
| 4900 | IS2 transposase TnpB | | * | * | CLRAG_34250 |
| 4901 | transposase | | * | * | CLRAG_34260 |
| 4902 | transposase | | * | * | CLRAG_34270 |
| 4903 | formate dehydrogenase H | | * | * | CLRAG_34290 |
| 4904 | hypothetical protein | | * | * | CLRAG_34320 |
| 4905 | hypothetical protein | | * | * | CLRAG_34330 |
| 4906 | hypothetical protein | | * | * | CLRAG_34340 |
| 4907 | hypothetical protein | | * | * | CLRAG_35240 |
| 4908 | hypothetical protein | | * | * | CLRAG_35800 |
| 4909 | carboxylesterase NlhH | | * | * | CLRAG_35840 |
| 4910 | group II intron, maturase-specific domain | | * | * | CLRAG_36010 |
| 4911 | type-1 restriction enzyme EcoKI specificity protein | | * | * | CLRAG_36030 |
| 4912 | hypothetical protein | | * | * | CLRAG_36070 |
| 4913 | acetyltransferase (GNAT) family protein | | * | * | CLRAG_36080 |
| 4914 | hypothetical protein | | * | * | CLRAG_36160 |
| 4915 | hypothetical protein | | * | * | CLRAG_36170 |
| 4916 | type III restriction enzyme, res subunit | | * | * | CLRAG_36180 |
| 4917 | RecBCD enzyme subunit RecD | | * | * | CLRAG_36190 |
| 4918 | phage integrase family protein | | * | * | CLRAG_36200 |
| 4919 | FRG domain protein | | * | * | CLRAG_36210 |
| 4920 | serine/threonine exchanger SteT | | * | * | CLRAG_36480 |
| 4921 | hypothetical protein | | * | * | CLRAG_36660 |
| 4922 | hypothetical protein | | * | * | CLRAG_37100 |
| 4923 | putative metal chaperone YciC | | * | * | CLRAG_37520 |
| 4924 | acetyltransferase (GNAT) family protein | | * | * | CLRAG_37540 |
| 4925 | chagasin family peptidase inhibitor I42 | | * | * | CLRAG_37550 |
| 4926 | hypothetical protein | | * | * | CLRAG_37560 |
| 4927 | hypothetical protein | | * | * | CLRAG_37580 |
| 4928 | hypothetical protein | | * | * | CLRAG_37590 |
| 4929 | hypothetical protein | | * | * | CLRAG_37600 |
| 4930 | hypothetical protein | | * | * | CLRAG_37630 |
| 4931 | hypothetical protein | | * | * | CLRAG_37900 |
| 4932 | N-acetylmuramoyl-L-alanine amidase LytC | | * | * | CLRAG_38010 |
| | precursor | | | | |
| 4933 | internalin B precursor | | * | * | CLRAG_38020 |
| 4934 | putative transposase, YhgA-like | | * | * | CLRAG_38240 |
| 4935 | GDP-mannose 4,6-dehydratase | | * | * | CLRAG_38610 |
| 4936 | GDP-L-fucose synthase | | * | * | CLRAG_38620 |
| 4937 | putative N-acetylmannosaminyltransferase | | * | * | CLRAG_38630 |
| 4938 | galactoside O-acetyltransferase | | * | * | CLRAG_38640 |
| 4939 | spore coat protein SA | | * | * | CLRAG_38650 |
| 4940 | hypothetical protein | | * | * | CLRAG_38660 |
| 4941 | hypothetical protein | | * | * | CLRAG_38670 |
| 4942 | D-inositol 3-phosphate glycosyltransferase | | * | * | CLRAG_38680 |
| 4943 | putative glycosyl transferase | | * | * | CLRAG_38690 |
| 4944 | hypothetical protein | | * | * | CLRAG_38700 |
| 4945 | hypothetical protein | | * | * | CLRAG_38710 |
| 4946 | glycosyl transferase family 11 | | * | * | CLRAG_38720 |
| 4947 | polysaccharide biosynthesis protein | | * | * | CLRAG_38730 |
| 4948 | UDP-glucose 6-dehydrogenase YwqF | | * | * | CLRAG_38740 |
| 4949 | UDP-glucose 4-epimerase | | * | * | CLRAG_38760 |
| 4950 | acyltransferase family protein | | * | * | CLRAG_38770 |
| 4951 | hypothetical protein | | * | * | CLRAG_38790 |
| 4952 | putative acetyltransferase | | * | * | CLRAG_38800 |
| 4953 | hypothetical protein | | * | * | CLRAG_38810 |
| 4954 | acyltransferase family protein | | * | * | CLRAG_38820 |
| 4955 | hypothetical protein | | * | * | CLRAG_38850 |
| 4956 | hypothetical protein | | * | * | CLRAG_38860 |
| 4957 | hypothetical protein | | * | * | CLRAG_39020 |
| 4958 | p-aminobenzoyl-glutamate hydrolase subunit B | | * | * | CLRAG_39040 |
| 4959 | sensor histidine kinase YycG | | * | * | CLRAG_39300 |
| 4960 | ABC-2 family transporter protein | | * | * | CLRAG_39320 |
| 4961 | ABC-2 family transporter protein | | * | * | CLRAG_39330 |
| 4962 | epoxyqueuosine reductase | | * | * | CLRAG_39350 |
| 4963 | ATP-dependent RNA helicase RhlE | | * | * | CLRAG_39540 |
| 4964 | hypothetical protein | | * | * | CLRAG_39580 |
| 4965 | HTH-type transcriptional regulator CysL | | * | * | CLRAG_39800 |
| 4966 | molybdenum-pterin-binding protein MopA | | * | * | CLRAG_39810 |
| 4967 | methionine gamma-lyase | | * | * | CLRAG_39890 |
| 4968 | putative transposase DNA-binding domain protein | | * | * | CLRAG_39910 |
| 4969 | putative transposase | | * | * | CLRAG_39920 |
| 4970 | helix-turn-helix domain protein | | * | * | CLRAG_39930 |
| 4971 | ferrous iron transport protein B | | * | * | CLRAG_39960 |
| 4972 | FeoA domain protein | | * | * | CLRAG_39970 |
| 4973 | hypothetical protein | | * | * | CLRAG_39990 |
| 4974 | hypothetical protein | | * | * | CLRAG_40000 |
| 4975 | hypothetical protein | | * | * | CLRAG_40110 |
| 4976 | putative glycosyl transferase | | * | * | CLRAG_40180 |

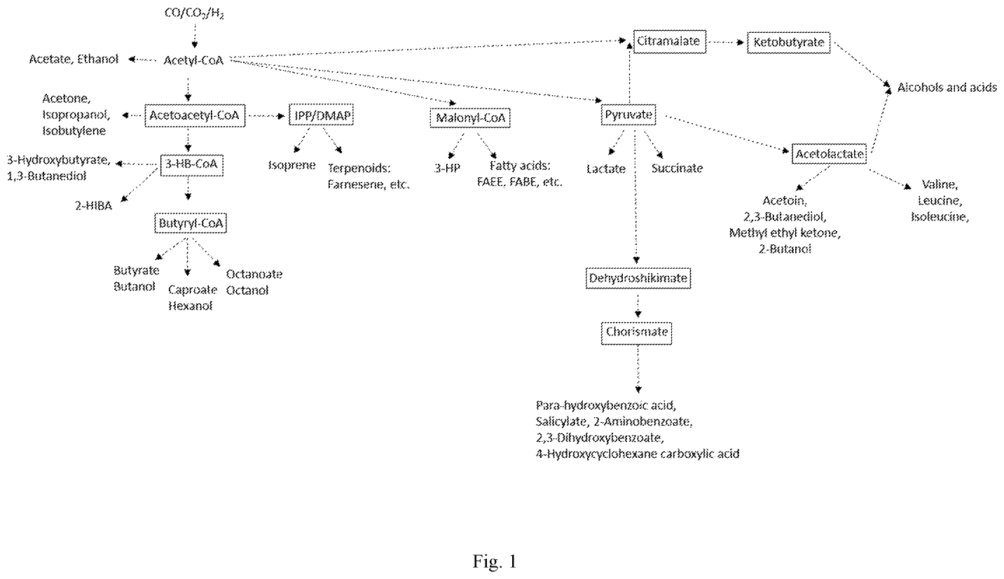

The inventors have further identified key metabolic pathways and key metabolic nodes in Wood-Ljungdahl microorganisms (FIG. 1). The invention further provides microorgansims with disrupted genes to strategically divert carbon flux is away from nonessential or undesirable metabolic nodes and through target metabolic nodes. Such strains have improved production of products downstream of those target metabolic nodes.

The invention finally provides methods of producting products by culturing the microorganism of the invention in the presence of a substrate, such as a gaseous substrate comprising one or more of CO, CO₂, and/or H₂. Possible combinations of disrupted genes for optimizing production of particular products are described in Examples 2-19.

As described elsewhere in this application, such products may include native or non-native products of Wood-Ljungdahl microorganisms. For example, such products include, but are not limited to acetyl-CoA, ethanol, acetate, butanol, butyrate, butyryl-CoA, 2,3-butanediol, lactate, butene, butadiene, methyl ethyl ketone, ethylene, acetone, isopropanol, lipids, 3-hydroxypropionate (3-HP), isoprene, farnesene, fatty acids (fatty acid ethyl esters, fatty acid butyl esters), 2-butanol, 1,2-propanediol, 1-propanol, chorismate-derived products, 3-hydroxybutyrate, 1,3-butanediol, C6-C8 alcohols (hexanol, heptanol, octanol), caproate, octanoate, isopentenyl pyrophosphate (IPP), dimethylallyl pyrophosphate (DMAPP), acetoacetyl-CoA, 3-hydroxybutyrate-CoA (3-HB-CoA), malonyl-CoA, pyruvate, dehydroshikimate, chorismate, para-hydroxybenzoic acid, salicylate, 2-aminobenzoate, 2,3-dihydroxybenzoate, 2-hydroxycyclohexane carboxylic acid, citramalate, ketobutyrate, acetolactate, acetoin, valine, leucine, and isoleucine.

### EXAMPLES

The following examples further illustrate the invention but, of course, should not be construed to limit its scope in any way.

### Example 1

This example describes metabolic modeling in Wood-Ljungdahl microorganisms.

A genome-scale metabolic model of Clostridium autoethanogenum like the one described by Marcellin, Green Chem, 18: 3020-3028, 2016 was utilized. This model was used to simulate the design, construction, in silico growth and screening of strains with disruptive gene mutations to predict those that would produce higher yields of native compounds. In addition, new genome-scale models were built for a number non-native compound-producing strains. For these, heterologous genes and metabolic reactions were added to the wild type Clostridium autoethanogenum model structure to represent the incorporation of the non-native compound production pathway. Although the model used for the experimental work described herein is based on Clostridium autoethanogenum, the results can reasonably be expected to apply to other Wood-Ljungdahl microorganisms as well, given similarities in metabolism.

For each chemical production strain, millions of mutant strains incorporating different combinations of disruptive gene mutations were built in silico. Boolean gene-protein-reaction associations were used to determine which metabolic reactions were inactivated upon disruption of a gene (Thiele, Nature Protocols, 5: 93-121, 2010). The design, construction and screening of mutant strains was carried out using cameo version 0.11.2 (Sonnenschein, Biosustain/Cameo: 0.11.0, doi: 10.5281/zenodo.835730, 2017) and evolutionary algorithms implemented by inspyred version 1.0.1.

The growth of these mutant strains was simulated using two constraint-based computational modeling techniques: flux balance analysis (FBA) and linear minimization of metabolic adjustment (LMOMA). These growth simulation techniques are used to capture two likely metabolic phenotypes, following genetic perturbation (Maia, Proceedings of the Genetic and Evolutionary Computation Conference Companion on—GECCO '17, New York, N.Y., ACM Press, 1661-1668, 2017). An experimental metabolic flux profile was constructed and used as the reference state for LMOMA simulations. Growth simulations were run using scripts from cobrapy version 0.8.2 (Ebrahim., COBRApy: COnstraints-Based Reconstruction and Analysis for Python, BMC Syst Biol, 7: 74, 2013), with optlang version 1.2.3 (Jensen, Optlang: An Algebraic Modeling Language for Mathematical Optimization,” The Journal of Open Source Software, 2, doi:10.21105/joss.00139, 2017) as the solver interface and Gurobi Optimizer version 7.0.2 as the optimization solver.

Growth rates and key metabolic fluxes including those for fermentation products were recorded and used to screen strains. For each strain simulation, the biomass-product coupled yield (BPCY) and the and the carbon molar yield were calculated. These yields were used to determine the fitness score.

In addition, flux variability analysis (FVA) was carried out to determine whether the mutant strain requires production of the compound of interest for growth to occur (growth-coupled strain designs). If the minimum boundary flux of the compound of interest was greater than zero during growth, the strain was classified as growth-coupling. These growth-coupled strain designs should allow greater fermentation stability during continuous fermentation. This minimum flux was converted to carbon yield (FVA minimum yield) and used to compare the level of growth coupling between strains.

### Example 2

This example describes disruptions for improved production of acetate in Wood-Ljungdahl microorganisms. Metabolic modeling experiments were performed as described in Example 1. Acetate is a native product of Wood-Ljungdahl microorganisms.

| | | # | | | FVA | |
|---|---|---|---|---|---|---|
| | | Disrupted | Disrupted | Disrupted | minimum | Fitness |
| # | Technique | genes | genes | reactions | yield | score |
| 1 | Yield - | 6 | CAETHG_1371, | Adenosine 5′-monophosphate phosphohydrolase (H2O + | 0.327282 | 0.093692 |
| | LMOMA | | CAETHG_2721, | AMP <=> Phosphate + H+ + Adenosine), | | |
| | | | CAETHG_2753, | Phosphoenolpyruvate carboxykinase (PPCK) (ATP + | | |
| | | | CAETHG_2754, | Oxaloacetate + H+ --> ADP + CO2 + Phosphoenolpyruvate), | | |
| | | | CAETHG_3293, | Isocitrate dehydrogenase (NAD + Isocitrate <=> NADH + | | |
| | | | CAETHG_0909 | CO2 + 2-Oxoglutarate + H+), Homocysteine synthase (O- | | |
| | | | | acetylhomoserine sulfhydrolase) (H2S + O-Acetyl-L- | | |
| | | | | homoserine --> Acetate + H+ + Homocysteine), Cysteine | | |
| | | | | desulfhydrase (H2O + L-Cysteine <=> NH3 + Pyruvate + | | |
| | | | | H2S), Prephenate:NAD + oxidoreductase(decarboxylating) | | |
| | | | | (NAD + Prephenate --> NADH + CO2 + p- | | |
| | | | | hydroxyphenylpyruvate) | | |
| 2 | Yield - | 5 | CAETHG_1371, | Adenosine 5′-monophosphate phosphohydrolase (H2O + | 0.327819 | 0.093362 |
| | LMOMA | | CAETHG_2721, | AMP <=> Phosphate + H+ + Adenosine), | | |
| | | | CAETHG_2753, | Phosphoenolpyruvate carboxykinase (PPCK) (ATP + | | |
| | | | CAETHG_2754, | Oxaloacetate + H+ --> ADP + CO2 + Phosphoenolpyruvate), | | |
| | | | CAETHG_3293 | Isocitrate dehydrogenase (NAD + Isocitrate <=> NADH + | | |
| | | | | CO2 + 2-Oxoglutarate + H+), Homocysteine synthase (O- | | |
| | | | | acetylhomoserine sulfhydrolase) (H2S + O-Acetyl-L- | | |
| | | | | homoserine --> Acetate + H+ + Homocysteine), Cysteine | | |
| | | | | desulfhydrase (H2O + L-Cysteine <=> NH3 + Pyruvate + H2S) | | |
| 3 | Yield - | 6 | CAETHG_1371, | Adenosine 5′-monophosphate phosphohydrolase (H2O + | 0.326972 | 0.09307 |
| | LMOMA | | CAETHG_2721, | AMP <=> Phosphate + H+ + Adenosine), | | |
| | | | CAETHG_2751, | Phosphoenolpyruvate carboxykinase (PPCK) (ATP + | | |
| | | | CAETHG_2753, | Oxaloacetate + H+ --> ADP + CO2 + Phosphoenolpyruvate), | | |
| | | | CAETHG_3293, | Citramalate synthase (H2O + Pyruvate + Acetyl-CoA <=> | | |
| | | | CAETHG_0909 | CoA + Citramalate), Isocitrate dehydrogenase (NAD + | | |
| | | | | Isocitrate <=> NADH + CO2 + 2-Oxoglutarate + H+), Cysteine | | |
| | | | | desulfhydrase (H2O + L-Cysteine <=> NH3 + Pyruvate + | | |
| | | | | H2S), Prephenate:NAD + oxidoreductase(decarboxylating) | | |
| | | | | (NAD + Prephenate --> NADH + CO2 + p- | | |
| | | | | hydroxyphenylpyruvate) | | |
| 4 | Yield - | 5 | CAETHG_1371, | Adenosine 5′-monophosphate phosphohydrolase (H2O + | 0.326957 | 0.092998 |
| | LMOMA | | CAETHG_2721, | AMP <=> Phosphate + H+ + Adenosine), | | |
| | | | CAETHG_2753, | Phosphoenolpyruvate carboxykinase (PPCK) (ATP + | | |
| | | | CAETHG_3293, | Oxaloacetate + H+ --> ADP + CO2 + Phosphoenolpyruvate), | | |
| | | | CAETHG_0909 | Isocitrate dehydrogenase (NAD + Isocitrate <=> NADH + | | |
| | | | | CO2 + 2-Oxoglutarate + H+), Cysteine desulfhydrase (H2O + | | |
| | | | | L-Cysteine <=> NH3 + Pyruvate + H2S), Prephenate:NAD + | | |
| | | | | oxidoreductase(decarboxylating) (NAD + Prephenate --> | | |
| | | | | NADH + CO2 + p-hydroxyphenylpyruvate) | | |
| 5 | Yield - | 4 | CAETHG_0233, | 4-imidazolone-5-propanoate amidohydrolase (H2O + 4- | 0.322658 | 0.092572 |
| | LMOMA | | CAETHG_2753, | Imidazolone-5-propanoate --> N-Formimino-L-glutamate), | | |
| | | | CAETHG_2932, | Isocitrate dehydrogenase (NAD + Isocitrate <=> NADH + | | |
| | | | CAETHG_3293 | CO2 + 2-Oxoglutarate + H+), Alpha-acetolactate | | |
| | | | | decarboxylase (ALCTT --> CO2 + (R)-Acetoin), Cysteine | | |
| | | | | desulfhydrase (H2O + L-Cysteine <=> NH3 + Pyruvate + H2S) | | |
| 6 | Yield - | 4 | CAETHG_2753, | Isocitrate dehydrogenase (NAD + Isocitrate <=> NADH + | 0.322819 | 0.092318 |
| | LMOMA | | CAETHG_2932, | CO2 + 2-Oxoglutarate + H+), Alpha-acetolactate | | |
| | | | CAETHG_3021, | decarboxylase (ALCTT --> CO2 + (R)-Acetoin), L-Arginine | | |
| | | | CAETHG_3293 | iminohydrolase (H2O + L-Arginine <=> NH3 + Citrulline), | | |
| | | | | Cysteine desulfhydrase (H2O + L-Cysteine <=> NH3 + | | |
| | | | | Pyruvate + H2S) | | |
| 7 | Yield - | 3 | CAETHG_2753, | Isocitrate dehydrogenase (NAD + Isocitrate <=> NADH + | 0.322658 | 0.09231 |
| | LMOMA | | CAETHG_2932, | CO2 + 2-Oxoglutarate + H+), Alpha-acetolactate | | |
| | | | CAETHG_3293 | decarboxylase (ALCTT --> CO2 + (R)-Acetoin), Cysteine | | |
| | | | | desulfhydrase (H2O + L-Cysteine <=> NH3 + Pyruvate + H2S) | | |
| 8 | Yield - | 3 | CAETHG_2753, | Isocitrate dehydrogenase (NAD + Isocitrate <=> NADH + | 0.325822 | 0.091762 |
| | LMOMA | | CAETHG_3293, | CO2 + 2-Oxoglutarate + H+), Cysteine desulfhydrase (H2O + | | |
| | | | CAETHG_3924 | L-Cysteine <=> NH3 + Pyruvate + H2S), D-Ribose 1,5- | | |
| | | | | phosphomutase (Ribose 1-phosphate <=> ribose-5- | | |
| | | | | phosphate) | | |
| 9 | Yield - | 3 | CAETHG_0160, | N-Ribosylnicotinamide:orthophosphate ribosyltransferase | 0.325822 | 0.091762 |
| | LMOMA | | CAETHG_2753, | (Phosphate + N-Ribosylnicotinamide <=> Nicotinamide + | | |
| | | | CAETHG_3293 | Ribose 1-phosphate), Isocitrate dehydrogenase (NAD + | | |
| | | | | Isocitrate <=> NADH + CO2 + 2-Oxoglutarate + H+), Cysteine | | |
| | | | | desulfhydrase (H2O + L-Cysteine <=> NH3 + Pyruvate + H2S) | | |
| 10 | Yield - | 3 | CAETHG_1371, | Adenosine 5′-monophosphate phosphohydrolase (H2O + | 0.325822 | 0.091762 |
| | LMOMA | | CAETHG_2753, | AMP <=> Phosphate + H+ + Adenosine), Isocitrate | | |
| | | | CAETHG_3293 | dehydrogenase (NAD + Isocitrate <=> NADH + CO2 + 2- | | |
| | | | | Oxoglutarate + H+), Cysteine desulfhydrase (H2O + L- | | |
| | | | | Cysteine <=> NH3 + Pyruvate + H2S) | | |
| 11 | Yield - | 3 | CAETHG_2721, | Phosphoenolpyruvate carboxykinase (PPCK) (ATP + | 0.324411 | 0.0913 |
| | LMOMA | | CAETHG_2753, | Oxaloacetate + H+ --> ADP + CO2 + Phosphoenolpyruvate), | | |
| | | | CAETHG_3293 | Isocitrate dehydrogenase (NAD + Isocitrate <=> NADH + | | |
| | | | | CO2 + 2-Oxoglutarate + H+), Cysteine desulfhydrase (H2O + | | |
| | | | | L-Cysteine <=> NH3 + Pyruvate + H2S) | | |
| 12 | Yield - | 3 | CAETHG_2721, | Phosphoenolpyruvate carboxykinase (PPCK) (ATP + | 0.327495 | 0.090854 |
| | LMOMA | | CAETHG_3293, | Oxaloacetate + H+ --> ADP + CO2 + Phosphoenolpyruvate), | | |
| | | | CAETHG_3924 | Cysteine desulfhydrase (H2O + L-Cysteine <=> NH3 + | | |
| | | | | Pyruvate + H2S), D-Ribose 1,5-phosphomutase (Ribose 1- | | |
| | | | | phosphate <=> ribose-5-phosphate) | | |
| 13 | Yield - | 3 | CAETHG_2751, | Citramalate synthase (H2O + Pyruvate + Acetyl-CoA <=> | 0.322674 | 0.090674 |
| | LMOMA | | CAETHG_2753, | CoA + Citramalate), Isocitrate dehydrogenase (NAD + | | |
| | | | CAETHG_3293 | Isocitrate <=> NADH + CO2 + 2-Oxoglutarate + H+), Cysteine | | |
| | | | | desulfhydrase (H2O + L-Cysteine <=> NH3 + Pyruvate + H2S) | | |
| 14 | Yield - | 3 | CAETHG_0233, | 4-imidazolone-5-propanoate amidohydrolase (H2O + 4- | 0.322658 | 0.09027 |
| | LMOMA | | CAETHG_2753, | Imidazolone-5-propanoate --> N-Formimino-L-glutamate), | | |
| | | | CAETHG_3293 | Isocitrate dehydrogenase (NAD + Isocitrate <=> NADH + | | |
| | | | | CO2 + 2-Oxoglutarate + H+), Cysteine desulfhydrase (H2O + | | |
| | | | | L-Cysteine <=> NH3 + Pyruvate + H2S) | | |
| 15 | Yield - | 3 | CAETHG_0234, | 4,5-Dihydro-4-oxo-5-imidazolepropanoate hydro-lyase (4- | 0.322658 | 0.09027 |
| | LMOMA | | CAETHG_2753, | Imidazolone-5-propanoate <=> H2O + Urocanate), | | |
| | | | CAETHG_3293 | Isocitrate dehydrogenase (NAD + Isocitrate <=> NADH + | | |
| | | | | CO2 + 2-Oxoglutarate + H+), Cysteine desulfhydrase (H2O + | | |
| | | | | L-Cysteine <=> NH3 + Pyruvate + H2S) | | |
| 16 | Yield - | 3 | CAETHG_2753, | Isocitrate dehydrogenase (NAD + Isocitrate <=> NADH + | 0.322819 | 0.090256 |
| | LMOMA | | CAETHG_3021, | CO2 + 2-Oxoglutarate + H+), L-Arginine iminohydrolase | | |
| | | | CAETHG_3293 | (H2O + L-Arginine <=> NH3 + Citrulline), Cysteine | | |
| | | | | desulfhydrase (H2O + L-Cysteine <=> NH3 + Pyruvate + H2S) | | |
| 17 | Yield - | 2 | CAETHG_2753, | Isocitrate dehydrogenase (NAD + Isocitrate <=> NADH + | 0.322658 | 0.090044 |
| | LMOMA | | CAETHG_3293 | CO2 + 2-Oxoglutarate + H+), Cysteine desulfhydrase (H2O + | | |
| 18 | Yield - | 2 | CAETHG_2932, | Alpha-acetolactate decarboxylase (ALCTT --> CO2 + (R)- | 0.322658 | 0.089638 |
| | LMOMA | | CAETHG_3293 | Acetoin), Cysteine desulfhydrase (H2O + L-Cysteine <=> | | |
| | | | | NH3 + Pyruvate + H2S) | | |
| 19 | Yield - | 2 | CAETHG_0160, | N-Ribosylnicotinamide:orthophosphate ribosyltransferase | 0.325822 | 0.0885 |
| | LMOMA | | CAETHG_3293 | (Phosphate + N-Ribosylnicotinamide <=> Nicotinamide + | | |
| | | | | Ribose 1-phosphate), Cysteine desulfhydrase (H2O + L- | | |
| | | | | Cysteine <=> NH3 + Pyruvate + H2S) | | |
| 20 | Yield - | 2 | CAETHG_0160, | N-Ribosylnicotinamide:orthophosphate ribosyltransferase | 0.328306 | 0.087946 |
| | LMOMA | | CAETHG_0909 | (Phosphate + N-Ribosylnicotinamide <=> Nicotinamide + | | |
| | | | | Ribose 1-phosphate), Prephenate:NAD + | | |
| | | | | oxidoreductase(decarboxylating) (NAD + Prephenate --> | | |
| | | | | NADH + CO2 + p-hydroxyphenylpyruvate) | | |
| 21 | Yield - | 2 | CAETHG_1270, | AMP:pyrophosphate phosphoribosyltransferase (PPi + | 0.328306 | 0.087946 |
| | LMOMA | | CAETHG_0909 | AMP <=> PRPP + Adenine), Prephenate:NAD + | | |
| | | | | oxidoreductase(decarboxylating) (NAD + Prephenate --> | | |
| | | | | NADH + CO2 + p-hydroxyphenylpyruvate) | | |
| 22 | Yield - | 2 | CAETHG_0160, | N-Ribosylnicotinamide:orthophosphate ribosyltransferase | 0.330562 | 0.08744 |
| | LMOMA | | CAETHG_2721 | (Phosphate + N-Ribosylnicotinamide <=> Nicotinamide + | | |
| | | | | Ribose 1-phosphate), Phosphoenolpyruvate carboxykinase | | |
| | | | | (PPCK) (ATP + Oxaloacetate + H+ --> ADP + CO2 + | | |
| | | | | Phosphoenolpyruvate) | | |
| 23 | Yield - | 1 | CAETHG_0909 | Prephenate:NAD + oxidoreductase(decarboxylating) (NAD + | 0.32509 | 0.0873 |
| | LMOMA | | | Prephenate --> NADH + CO2 + p-hydroxyphenylpyruvate) | | |
| 24 | Yield - | 1 | CAETHG_0160 | N-Ribosylnicotinamide:orthophosphate ribosyltransferase | 0.32887 | 0.087246 |
| | LMOMA | | | (Phosphate + N-Ribosylnicotinamide <=> Nicotinamide + | | |
| | | | | Ribose 1-phosphate) | | |
| 25 | Yield - | 1 | CAETHG_1371 | Adenosine 5′-monophosphate phosphohydrolase (H2O + | 0.32887 | 0.087246 |
| | LMOMA | | | AMP <=> Phosphate + H+ + Adenosine) | | |
| 26 | Yield - | 1 | CAETHG_1270 | AMP:pyrophosphate phosphoribosyltransferase (PPi + | 0.32887 | 0.087246 |
| | LMOMA | | | AMP <=> PRPP + Adenine) | | |
| 27 | Yield - | 1 | CAETHG_3924 | D-Ribose 1,5-phosphomutase (Ribose 1-phosphate <=> | 0.32887 | 0.087246 |
| | LMOMA | | | ribose-5-phosphate) | | |
| 28 | Yield - | 1 | CAETHG_2721 | Phosphoenolpyruvate carboxykinase (PPCK) (ATP + | 0.327443 | 0.0872 |
| | LMOMA | | | Oxaloacetate + H+ --> ADP + CO2 + Phosphoenolpyruvate) | | |
| 29 | Yield - | 1 | CAETHG_3021 | L-Arginine iminohydrolase (H2O + L-Arginine <=> NH3 + | 0.325831 | 0.08702 |
| | LMOMA | | | Citrulline) | | |

### Example 3

This example describes disruptions for improved production of ethanol in Wood-Ljungdahl microorganisms. Metabolic modeling experiments were performed as described in Example 1. Ethanol is a native product of Wood-Ljungdahl microorganisms.

| | | # | | | FVA | |
|---|---|---|---|---|---|---|
| | | Disrupted | Disrupted | Disrupted | minimum | Fitness |
| # | Technique | genes | genes | reactions | yield | score |
| 1 | Yield - | 4 | CAETHG_2932, | Alpha-acetolactate decarboxylase (ALCTT --> CO2 + (R)- | 0.22708 | 0.205528 |
| | LMOMA | | CAETHG_3359, | Acetoin), ATP:acetate phosphotransferase (ATP + H+ + | | |
| | | | CAETHG_3510, | Propionate --> ADP + Propionyl phosphate), 2,6- | | |
| | | | CAETHG_0686 | Diaminoheptanedioate:2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate), L-Threonine | | |
| | | | | acetaldehyde-lyase (L-Threonine --> Glycine + Acetaldehyde) | | |
| 2 | Yield - | 5 | CAETHG_2932, | Alpha-acetolactate decarboxylase (ALCTT --> CO2 + (R)- | 0.230473 | 0.205316 |
| | LMOMA | | CAETHG_3021, | Acetoin), L-Arginine iminohydrolase (H2O + L-Arginine <=> | | |
| | | | CAETHG_3359, | NH3 + Citrulline), ATP:acetate phosphotransferase (ATP + | | |
| | | | CAETHG_3924, | H+ + Propionate --> ADP + Propionyl phosphate), D-Ribose | | |
| | | | CAETHG_0498 | 1,5-phosphomutase (Ribose 1-phosphate <=> ribose-5- | | |
| | | | | phosphate), Cystathionine beta lyase (H2O + Cystathionine --> | | |
| | | | | NH3 + Pyruvate + Homocysteine) | | |
| 3 | Yield - | 5 | CAETHG_1270, | AMP:pyrophosphate phosphoribosyltransferase (PPi + AMP <=> | 0.230473 | 0.205316 |
| | LMOMA | | CAETHG_2932, | PRPP + Adenine), Alpha-acetolactate decarboxylase | | |
| | | | CAETHG_3021, | (ALCTT --> CO2 + (R)-Acetoin), L-Arginine iminohydrolase | | |
| | | | CAETHG_3359, | (H2O + L-Arginine <=> NH3 + Citrulline), ATP:acetate | | |
| | | | CAETHG_0498 | phosphotransferase (ATP + H+ + Propionate --> ADP + | | |
| | | | | Propionyl phosphate), Cystathionine beta lyase (H2O + | | |
| | | | | Cystathionine --> NH3 + Pyruvate + Homocysteine) | | |
| 4 | Yield - | 4 | CAETHG_2932, | Alpha-acetolactate decarboxylase (ALCTT --> CO2 + (R)- | 0.230404 | 0.205306 |
| | LMOMA | | CAETHG_3359, | Acetoin), ATP:acetate phosphotransferase (ATP + H+ + | | |
| | | | CAETHG_3924, | Propionate --> ADP + Propionyl phosphate), D-Ribose 1,5- | | |
| | | | CAETHG_0498 | phosphomutase (Ribose 1-phosphate <=> ribose-5- | | |
| | | | | phosphate), Cystathionine beta lyase (H2O + Cystathionine --> | | |
| | | | | NH3 + Pyruvate + Homocysteine) | | |
| 5 | Yield - | 4 | CAETHG_0160, | N-Ribosylnicotinamide:orthophosphate ribosyltransferase | 0.230404 | 0.205306 |
| | LMOMA | | CAETHG_2932, | (Phosphate + N-Ribosylnicotinamide <=> Nicotinamide + | | |
| | | | CAETHG_3359, | Ribose 1-phosphate), Alpha-acetolactate decarboxylase | | |
| | | | CAETHG_0498 | (ALCTT --> CO2 + (R)-Acetoin), ATP:acetate | | |
| | | | | phosphotransferase (ATP + H+ + Propionate --> ADP + | | |
| | | | | Propionyl phosphate), Cystathionine beta lyase (H2O + | | |
| | | | | Cystathionine --> NH3 + Pyruvate + Homocysteine) | | |
| 6 | Yield - | 4 | CAETHG_1270, | AMP:pyrophosphate phosphoribosyltransferase (PPi + AMP <=> | 0.230404 | 0.205306 |
| | LMOMA | | CAETHG_2932, | PRPP + Adenine), Alpha-acetolactate decarboxylase | | |
| | | | CAETHG_3359, | (ALCTT --> CO2 + (R)-Acetoin), ATP:acetate | | |
| | | | CAETHG_0498 | phosphotransferase (ATP + H+ + Propionate --> ADP + | | |
| | | | | Propionyl phosphate), Cystathionine beta lyase (H2O + | | |
| | | | | Cystathionine --> NH3 + Pyruvate + Homocysteine) | | |
| 7 | Yield - | 3 | CAETHG_1371, | Adenosine 5′-monophosphate phosphohydrolase (H2O + | 0.230833 | 0.205248 |
| | LMOMA | | CAETHG_2932, | AMP <=> Phosphate + H+ + Adenosine), Alpha-acetolactate | | |
| | | | CAETHG_3358 | decarboxylase (ALCTT --> CO2 + (R)-Acetoin), Phosphate | | |
| | | | | transacetylase (Phosphate + Acetyl-CoA + H+ <=> CoA + | | |
| | | | | Acetylphosphate) | | |
| 8 | Yield - | 3 | CAETHG_2932, | Alpha-acetolactate decarboxylase (ALCTT --> CO2 + (R)- | 0.230833 | 0.205248 |
| | LMOMA | | CAETHG_3359, | Acetoin), ATP:acetate phosphotransferase (ATP + H+ + | | |
| | | | CAETHG_3924 | Propionate --> ADP + Propionyl phosphate), D-Ribose 1,5- | | |
| | | | | phosphomutase (Ribose 1-phosphate <=> ribose-5- | | |
| | | | | phosphate) | | |
| 9 | Yield - | 3 | CAETHG_0160, | N-Ribosylnicotinamide:orthophosphate ribosyltransferase | 0.230833 | 0.205248 |
| | LMOMA | | CAETHG_2932, | {Phosphate + N-Ribosylnicotinamide <=> Nicotinamide + | | |
| | | | CAETHG_3358 | Ribose 1-phosphate), Alpha-acetolactate decarboxylase | | |
| | | | | (ALCTT --> CO2 + (R)-Acetoin), Phosphate transacetylase | | |
| | | | | (Phosphate + Acetyl-CoA + H+ <=> CoA + Acetylphosphate) | | |
| 10 | Yield - | 3 | CAETHG_1270, | AMP:pyrophosphate phosphoribosyltransferase (PPi + | 0.230833 | 0.205248 |
| | LMOMA | | CAETHG_2932, | AMP <=> PRPP + Adenine), Alpha-acetolactate decarboxylase | | |
| | | | CAETHG_3358 | (ALCTT --> CO2 + (R)-Acetoin), Phosphate transacetylase | | |
| | | | | (Phosphate + Acetyl-CoA + H+ <=> CoA + Acetylphosphate) | | |
| 11 | Yield - | 3 | CAETHG_2932, | Alpha-acetolactate decarboxylase (ALCTT --> CO2 + (R)- | 0.230833 | 0.205248 |
| | LMOMA | | CAETHG_3358, | Acetoin), Phosphate transacetylase (Phosphate + Acetyl-CoA | | |
| | | | CAETHG_3924 | H+ <=> CoA + Acetylphosphate), D-Ribose 1,5-phosphomutase | | |
| | | | | (Ribose 1-phosphate <=> ribose-5-phosphate) | | |
| 12 | Yield - | 3 | CAETHG_2932, | Alpha-acetolactate decarboxylase (ALCTT --> CO2 + (R)- | 0.228988 | 0.205226 |
| | LMOMA | | CAETHG_3358, | Acetoin), Phosphate transacetylase (Phosphate + Acetyl-CoA + | | |
| | | | CAETHG_0498 | H+ <=> CoA + Acetylphosphate), Cystathionine beta lyase | | |
| | | | | (H2O + Cystathionine --> NH3 + Pyruvate + Homocysteine) | | |
| 13 | Yield - | 3 | CAETHG_2932, | Alpha-acetolactate decarboxylase (ALCTT --> CO2 + (R)- | 0.229481 | 0.205172 |
| | LMOMA | | CAETHG_3021, | Acetoin), L-Arginine iminohydrolase (H2O + L-Arginine <=> | | |
| | | | CAETHG_3358 | NH3 + Citrulline), Phosphate transacetylase (Phosphate + | | |
| | | | | Acetyl-CoA + H+ <=> CoA + Acetylphosphate) | | |
| 14 | Yield - | 2 | CAETHG_2932, | Alpha-acetolactate decarboxylase (ALCTT --> CO2 + (R)- | 0.229408 | 0.205162 |
| | LMOMA | | CAETHG_3359 | Acetoin), ATP:acetate phosphotransferase (ATP + H+ + | | |
| | | | | Propionate --> ADP + Propionyl phosphate) | | |
| 15 | Yield - | 2 | CAETHG_2932, | Alpha-acetolactate decarboxylase (ALCTT --> CO2 + (R)- | 0.229408 | 0.205162 |
| | LMOMA | | CAETHG_3358 | Acetoin), Phosphate transacetylase (Phosphate + Acetyl-CoA + | | |
| | | | | H+ <=> CoA + Acetylphosphate) | | |
| 16 | Yield - | 3 | CAETHG_2721, | Phosphoenolpyruvate carboxykinase (PPCK) (ATP + | 0.188313 | 0.194484 |
| | LMOMA | | CAETHG_2932, | Oxaloacetate + H+ --> ADP + CO2 + Phosphoenolpyruvate), | | |
| | | | CAETHG_3924 | Alpha-acetolactate decarboxylase (ALCTT --> CO2 + (R)- | | |
| | | | | Acetoin), D-Ribose 1,5-phosphomutase (Ribose 1- | | |
| | | | | phosphate <=> ribose-5-phosphate) | | |
| 17 | Yield - | 3 | CAETHG_3021, | L-Arginine iminohydrolase (H2O + L-Arginine <=> NH3 + | 0.221462 | 0.188812 |
| | LMOMA | | CAETHG_3358, | Citrulline), Phosphate transacetylase (Phosphate + Acetyl-CoA + | | |
| | | | CAETHG_3510 | H+ <=> CoA + Acetylphosphate), 2,6- | | |
| | | | | Diaminoheptanedioate:2-oxoglutarate aminotransferase (H2O + | | |
| | | | | L-Glutamate + H+ + tetrahydrodipicolinate <-- 2- | | |
| | | | | Oxoglutarate + LL-2,6-Diaminopimelate) | | |
| 18 | Yield - | 2 | CAETHG_3358, | Phosphate transacetylase (Phosphate + Acetyl-CoA + H+ <=> | 0.221395 | 0.188806 |
| | LMOMA | | CAETHG_3510 | CoA + Acetylphosphate), 2,6-Diaminoheptanedioate:2- | | |
| | | | | oxoglutarate aminotransferase (H2O + L-Glutamate + H+ + | | |
| | | | | tetrahydrodipicolinate <-- 2-Oxoglutarate + Ll-2,6- | | |
| | | | | Diaminopimelate) | | |
| 19 | Yield - | 3 | CAETHG_3358, | Phosphate transacetylase (Phosphate + Acetyl-CoA + H+ <=> | 0.230404 | 0.188442 |
| | LMOMA | | CAETHG_3924, | CoA + Acetylphosphate), D-Ribose 1,5-phosphomutase | | |
| | | | CAETHG_0498 | (Ribose 1-phosphate <=> ribose-5-phosphate), Cystathionine | | |
| | | | | beta lyase (H2O + Cystathionine --> NH3 + Pyruvate + | | |
| | | | | Homocysteine) | | |
| 20 | Yield - | 3 | CAETHG_0160, | N-Ribosylnicotinamide:orthophosphate ribosyltransferase | 0.230404 | 0.188442 |
| | LMOMA | | CAETHG_3358, | (Phosphate + N-Ribosylnicotinamide <=> Nicotinamide + | | |
| | | | CAETHG_0498 | Ribose 1-phosphate), Phosphate transacetylase (Phosphate + | | |
| | | | | Acetyl-CoA + H+ <=> CoA + Acetylphosphate), Cystathionine | | |
| | | | | beta lyase (H2O + Cystathionine --> NH3 + Pyruvate + | | |
| | | | | Homocysteine) | | |
| 21 | Yield - | 3 | CAETHG_1371, | Adenosine 5′-monophosphate phosphohydrolase (H2O + | 0.230404 | 0.188442 |
| | LMOMA | | CAETHG_3358, | AMP <=> Phosphate + H+ + Adenosine), Phosphate | | |
| | | | CAETHG_0498 | transacetylase (Phosphate + Acetyl-CoA + H+ <=> CoA + | | |
| | | | | Acetylphosphate), Cystathionine beta lyase (H2O + | | |
| | | | | Cystathionine --> NH3 Pyruvate + Homocysteine) | | |
| 22 | Yield - | 2 | CAETHG_3358, | Phosphate transacetylase (Phosphate + Acetyl-CoA + H+ <=> | 0.228988 | 0.188404 |
| | LMOMA | | CAETHG_0498 | CoA + Acetylphosphate), Cystathionine beta lyase (H2O + | | |
| | | | | Cystathionine --> NH3 + Pyruvate + Homocysteine) | | |
| 23 | Yield - | 2 | CAETHG_3359, | ATP:acetate phosphotransferase (ATP + H+ + Propionate --> | 0.228988 | 0.188404 |
| | LMOMA | | CAETHG_0498 | ADP + Propionyl phosphate), Cystathionine beta lyase (H2O + | | |
| | | | | Cystathionine --> NH3 + Pyruvate + Homocysteine) | | |
| 24 | Yield - | 3 | CAETHG_3021, | L-Arginine iminohydrolase (H2O + L-Arginine <=> NH3 + | 0.230902 | 0.188398 |
| | LMOMA | | CAETHG_3358, | Citrulline), Phosphate transacetylase (Phosphate + Acetyl-CoA + | | |
| | | | CAETHG_3924 | H+ <=> CoA + Acetylphosphate), D-Ribose 1,5- | | |
| | | | | phosphomutase (Ribose 1-phosphate <=> ribose-5- | | |
| | | | | phosphate) | | |
| 25 | Yield - | 2 | CAETHG_1270, | AMP:pyrophosphate phosphoribosyltransferase (PPi + | 0.230833 | 0.18839 |
| | LMOMA | | CAETHG_3359 | AMP <=> PRPP + Adenine), ATP:acetate phosphotransferase | | |
| | | | | (ATP + H+ + Propionate --> ADP + Propionyl phosphate) | | |
| 26 | Yield - | 2 | CAETHG_1371, | Adenosine 5′-monophosphate phosphohydrolase (H2O + | 0.230833 | 0.18839 |
| | LMOMA | | CAETHG_3359 | AMP <=> Phosphate + H+ + Adenosine), ATP:acetate | | |
| | | | | phosphotransferase (ATP + H+ + Propionate --> ADP + | | |
| | | | | Propionyl phosphate) | | |
| 27 | Yield - | 2 | CAETHG_3359, | ATP:acetate phosphotransferase (ATP + H+ + Propionate --> | 0.230833 | 0.18839 |
| | LMOMA | | CAETHG_3924 | ADP + Propionyl phosphate), D-Ribose 1,5-phosphomutase | | |
| | | | | (Ribose 1-phosphate <=> ribose-5-phosphate) | | |
| 28 | Yield - | 2 | CAETHG_1371, | Adenosine 5′-monophosphate phosphohydrolase (H2O + | 0.230833 | 0.18839 |
| | LMOMA | | CAETHG_3358 | AMP <=> Phosphate + H+ + Adenosine), Phosphate | | |
| | | | | transacetylase (Phosphate + Acetyl-CoA + H+ <=> CoA + | | |
| | | | | Acetylphosphate) | | |
| 29 | Yield - | 1 | CAETHG_3358 | Phosphate transacetylase (Phosphate + Acetyl-CoA + H+ <=> | 0.229408 | 0.188348 |
| | LMOMA | | | CoA + Acetylphosphate) | | |
| 30 | Yield - | 1 | CAETHG_3359 | ATP:acetate phosphotransferase (ATP + H+ + Propionate --> | 0.229408 | 0.188348 |
| | LMOMA | | | ADP + Propionyl phosphate) | | |
| 31 | Yield - | 2 | CAETHG_0160, | N-Ribosylnicotinamide:orthophosphate ribosyltransferase | 0.185926 | 0.186556 |
| | LMOMA | | CAETHG_2932 | (Phosphate + N-Ribosylnicotinamide <=> Nicotinamide + | | |
| | | | | Ribose 1-phosphate), Alpha-acetolactate decarboxylase | | |
| | | | | (ALCTT --> CO2 + (R)-Acetoin) | | |
| 32 | Yield - | 2 | CAETHG_2932, | Alpha-acetolactate decarboxylase (ALCTT --> CO2 + (R)- | 0.185926 | 0.186556 |
| | LMOMA | | CAETHG_3924 | Acetoin), D-Ribose 1,5-phosphomutase (Ribose 1- | | |
| | | | | phosphate <=> ribose-5-phosphate) | | |
| 33 | Yield - | 2 | CAETHG_2932, | Alpha-acetolactate decarboxylase {ALCTT --> CO2 + (R)- | 0.183754 | 0.184984 |
| | LMOMA | | CAETHG_3293 | Acetoin), Cysteine desulfhydrase (H2O + L-Cysteine <=> | | |
| | | | | NH3 + Pyruvate + H2S) | | |
| 34 | Yield - | 2 | CAETHG_2932, | Alpha-acetolactate decarboxylase (ALCTT --> CO2 + (R)- | 0.183906 | 0.184562 |
| | LMOMA | | CAETHG_0498 | Acetoin), Cystathionine beta lyase (H2O + Cystathionine --> | | |
| | | | | NH3 + Pyruvate + Homocysteine) | | |
| 35 | Yield - | 2 | CAETHG_2796, | NADP-dependent electron-bifurcating [FeFe]-hydrogenase | 0.183806 | 0.18449 |
| | LMOMA | | CAETHG_2932 | (Hyt) (NADP + Oxidizedferredoxin + 2.0H2 <=> NADPH + | | |
| | | | | 3.0H+ + Reducedferredoxin), Alpha-acetolactate decarboxylase | | |
| | | | | (ALCTT --> CO2 + (R)-Acetoin) | | |
| 36 | Yield - | 2 | CAETHG_2798, | NADP-dependent electron-bifurcating [FeFe]-hydrogenase | 0.183806 | 0.18449 |
| | LMOMA | | CAETHG_2932 | (Hyt) (NADP + Oxidizedferredoxin + 2.0H2 <=> NADPH + | | |
| | | | | 3.0H+ + Reducedferredoxin), Alpha-acetolactate decarboxylase | | |
| | | | | {ALCTT --> CO2 + (R)-Acetoin) | | |
| 37 | Yield - | 2 | CAETHG_2799, | NADP-dependent electron-bifurcating [FeFe]-hydrogenase | 0.183806 | 0.18449 |
| | LMOMA | | CAETHG_2932 | (Hyt) (NADP + Oxidizedferredoxin + 2.0H2 <=> NADPH + | | |
| | | | | 3.0H+ + Reducedferredoxin), Alpha-acetolactate decarboxylase | | |
| | | | | (ALCTT --> CO2 + (R)-Acetoin) | | |
| 38 | Yield - | 2 | CAETHG_2795, | NADP-dependent electron-bifurcating [FeFe]-hydrogenase | 0.183806 | 0.18449 |
| | LMOMA | | CAETHG 2932 | (Hyt) (NADP + Oxidizedferredoxin + 2.0H2 <=> NADPH + | | |
| | | | | 3.0H+ + Reducedferredoxin), Alpha-acetolactate decarboxylase | | |
| | | | | (ALCTT --> CO2 + (R)-Acetoin) | | |
| 39 | Yield - | 2 | CAETHG_2797, | NADP-dependent electron-bifurcating [FeFe]-hydrogenase | 0.183806 | 0.18449 |
| | LMOMA | | CAETHG_2932 | (Hyt) (NADP + Oxidizedferredoxin + 2.0H2 <=> NADPH + | | |
| | | | | 3.0H+ + Reducedferredoxin), Alpha-acetolactate decarboxylase | | |
| | | | | (ALCTT --> CO2 + (R)-Acetoin) | | |
| 40 | Yield - | 2 | CAETHG_0248, | L-lactate reversible transport via proton symport (H+_ext + L- | 0.183806 | 0.184038 |
| | LMOMA | | CAETHG_2932 | Lactate_ext <=> H+ + L-Lactate), Alpha-acetolactate | | |
| | | | | decarboxylase (ALCTT --> CO2 + (R)-Acetoin) | | |
| 41 | Yield - | 2 | CAETHG_2751, | Citramalate synthase (H2O + Pyruvate + Acetyl-CoA <=> | 0.183456 | 0.184024 |
| | LMOMA | | CAETHG 2932 | CoA + Citramalate), Alpha-acetolactate decarboxylase | | |
| | | | | (ALCTT --> CO2 + (R)-Acetoin) | | |
| 42 | Yield - | 2 | CAETHG_3924, | D-Ribose 1,5-phosphomutase (Ribose 1-phosphate <=> | 0.186014 | 0.183362 |
| | LMOMA | | CAETHG_0498 | ribose-5-phosphate), Cystathionine beta lyase (H2O + | | |
| | | | | Cystathionine --> NH3 + Pyruvate + Homocysteine) | | |
| 43 | Yield - | 1 | CAETHG_0160 | N-Ribosylnicotinamide:orthophosphate ribosyltransferase | 0.185926 | 0.182994 |
| | LMOMA | | | (Phosphate + N-Ribosylnicotinamide <=> Nicotinamide + | | |
| | | | | Ribose 1-phosphate) | | |
| 44 | Yield - | 1 | CAETHG_1270 | AMP:pyrophosphate phosphoribosyltransferase (PPi + | 0.185926 | 0.182994 |
| | LMOMA | | | AMP <=> PRPP + Adenine) | | |
| 45 | Yield - | 1 | CAETHG_3924 | D-Ribose 1,5-phosphomutase (Ribose 1-phosphate <=> | 0.185926 | 0.182994 |
| | LMOMA | | | ribose-5-phosphate) | | |
| 46 | Yield - | 1 | CAETHG_2721 | Phosphoenolpyruvate carboxykinase (PPCK) (ATP + | 0.185926 | 0.182756 |
| | LMOMA | | | Oxaloacetate + H+ --> ADP + CO2 + Phosphoenolpyruvate) | | |
| 47 | Yield - | 1 | CAETHG_3021 | L-Arginine iminohydrolase (H2O + L-Arginine <=> NH3 + | 0.186476 | 0.182602 |
| | LMOMA | | | Citrulline) | | |

### Example 4

This example describes disruptions for improved production of acetone in Wood-Ljungdahl microorganisms. Metabolic modeling experiments were performed as described in Example 1. Production of acetone in Wood-Ljungdahl microorganisms is described, e.g., in WO 2012/115527. The following pathway was used to model acetone production herein: 2.0 Acetyl-CoA-->CoA+Acetoacetyl-CoA; Acetate+Acetoacetyl-CoA-->Acetyl-CoA+Acetoacetate; Acetoacetate-->CO₂+Acetone; Acetone-->Acetone_ext.

| | | # | | | FVA | |
|---|---|---|---|---|---|---|
| | | Disrupted | Disrupted | Disrupted | minimum | Fitness |
| # | Technique | genes | genes | reactions | yield | score |
| 1 | Yield - | 5 | CAETHG_2721, | AcetyTCoAxarbon-dioxide ligase (ADP-forming) (ATP + | 0 | 0.057267 |
| | LMOMA | | CAETHG_2753, | Acetyl-CoA + H2CO3 --> ADP + Phosphate + H+ + Malonyl-CoA), | | |
| | | | CAETHG_3358, | Phosphoenolpyruvate carboxykinase (PPCK) (ATP + Oxaloacetate + | | |
| | | | CAETHG_3510, | H+ --> ADP + CO2 + Phosphoenolpyruvate), Isocitrate | | |
| | | | CAETHG_0909 | dehydrogenase (NAD + Isocitrate <=> NADH + CO2 + 2- | | |
| | | | | Oxoglutarate + H+), Phosphate transacetylase (Phosphate + Acetyl- | | |
| | | | | CoA + H+ <=> CoA + Acetylphosphate), 2,6- | | |
| | | | | Diaminoheptanedioate:2-oxoglutarate aminotransferase (H2O + L- | | |
| | | | | Glutamate + H+ + tetrahydrodipicolinate <-- 2-Oxoglutarate + LL- | | |
| | | | | 2,6-Diaminopimelate), Prephenate:NAD + | | |
| | | | | oxidoreductase(decarboxylating) (NAD + Prephenate --> NADH + | | |
| | | | | CO2 + p-hydroxyphenylpyruvate) | | |
| 2 | Yield - | 4 | CAETHG_2721, | Acetyl-CoA:carbon-dioxide ligase (ADP-forming) (ATP + Acetyl-CoA | 0 | 0.05724 |
| | LMOMA | | CAETHG_3358, | + H2CO3 --> ADP + Phosphate + H+ + Malonyl-CoA), | | |
| | | | CAETHG_3510, | Phosphoenolpyruvate carboxykinase (PPCK) (ATP + Oxaloacetate + | | |
| | | | CAETHG_0909 | H+ ~> ADP + CO2 + Phosphoenolpyruvate), Phosphate | | |
| | | | | transacetylase (Phosphate + Acetyl-CoA + H+ <=> CoA + | | |
| | | | | Acetylphosphate), 2,6-Diaminoheptanedioate:2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate), Prephenate:NAD + | | |
| | | | | oxidoreductase(decarboxylating) (NAD + Prephenate --> NADH + | | |
| | | | | CO2 + p-hydroxyphenylpyruvate) | | |
| 3 | Yield - | 2 | CAETHG_2721, | Acetyl-CoA:carbon-dioxide ligase (ADP-forming) (ATP + | 0 | 0.056697 |
| | LMOMA | | CAETHG_3359 | Acetyl-CoA + H2CO3 --> ADP + Phosphate + H+ + Malonyl-CoA), | | |
| | | | | Phosphoenolpyruvate carboxykinase (PPCK) (ATP + Oxaloacetate + | | |
| | | | | H+ --> ADP + CO2 + Phosphoenolpyruvate), ATP:acetate | | |
| | | | | phosphotransferase (ATP + H+ + Propionate --> ADP + Propionyl | | |
| | | | | phosphate) | | |
| 4 | Yield - | 3 | CAETHG_2721, | Phosphoenolpyruvate carboxykinase (PPCK) (ATP + Oxaloacetate + | 0 | 0.056481 |
| | LMOMA | | CAETHG_3359, | H+ --> ADP + CO2 + Phosphoenolpyruvate), ATP:acetate | | |
| | | | CAETHG_0909 | phosphotransferase (ATP + H+ + Propionate --> ADP + Propionyl | | |
| | | | | phosphate), Prephenate:NAD + oxidoreductase(decarboxylating) | | |
| | | | | (NAD + Prephenate --> NADH + CO2 + p-hydroxyphenylpyruvate) | | |
| 5 | Yield - | 3 | CAETHG_2721, | Phosphoenolpyruvate carboxykinase (PPCK) (ATP + Oxaloacetate + | 0 | 0.056292 |
| | LMOMA | | CAETHG_33S9, | H+ --> ADP + CO2 + Phosphoenolpyruvate), ATP:acetate | | |
| | | | CAETHG_3510 | phosphotransferase (ATP + H+ + Propionate --> ADP + Propionyl | | |
| | | | | phosphate), 2,6-Diaminoheptanedioate:2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 6 | Yield - | 3 | CAETHG_2721, | Phosphoenolpyruvate carboxykinase (PPCK) (ATP + Oxaloacetate + | 0 | 0.056154 |
| | LMOMA | | CAETHG_2753, | H+ --> ADP + CO2 + Phosphoenolpyruvate), Isocitrate | | |
| | | | CAETHG_3359 | dehydrogenase (NAD + Isocitrate <=> NADH + CO2 + 2- | | |
| | | | | Oxoglutarate + H+), ATP:acetate phosphotransferase (ATP + H+ + | | |
| | | | | Propionate --> ADP + Propionyl phosphate) | | |
| 7 | Yield - | 2 | CAETHG_2721, | Phosphoenolpyruvate carboxykinase (PPCK) (ATP + Oxaloacetate + | 0 | 0.056112 |
| | LMOMA | | CAETHG_3358 | H+ --> ADP + CO2 + Phosphoenolpyruvate), Phosphate | | |
| | | | | transacetylase (Phosphate + Acetyl-CoA + H+ <=> CoA + | | |
| | | | | Acetylphosphate) | | |
| 8 | Yield - | 2 | CAETHG_2721, | Phosphoenolpyruvate carboxykinase (PPCK) (ATP + Oxaloacetate + | 0 | 0.056112 |
| | LMOMA | | CAETHG 3359 | H+ --> ADP + CO2 + Phosphoenolpyruvate), ATP:acetate | | |
| | | | | phosphotransferase (ATP + H+ + Propionate --> ADP + Propionyl | | |
| | | | | phosphate) | | |
| 9 | Yield - | 2 | CAETHG_2721, | Phosphoenolpyruvate carboxykinase (PPCK) (ATP + Oxaloacetate + | 0 | 0.056022 |
| | LMOMA | | CAETHG_3359 | H+ --> ADP + CO2 + Phosphoenolpyruvate), ATP:acetate | | |
| | | | | phosphotransferase (ATP + H+ + Propionate --> ADP + Propionyl | | |
| | | | | phosphate) | | |
| 10 | Yield - | 2 | CAETHG_2932, | Alpha-acetolactate decarboxylase (ALCTT --> CO2 + (R)-Acetoin), | 0 | 0.055245 |
| | LMOMA | | CAETHG_3358 | Phosphate transacetylase (Phosphate + Acetyl-CoA + H+ <=> CoA + | | |
| | | | | Acetylphosphate) | | |
| 11 | Yield - | 2 | CAETHG_2932, | Alpha-acetolactate decarboxylase (ALCTT --> CO2 + (R)-Acetoin), | 0 | 0.055245 |
| | LMOMA | | CAETHG_3359 | ATPiacetate phosphotransferase (ATP + H+ + Propionate --> ADP + | | |
| | | | | Propionyl phosphate) | | |
| 12 | Yield - | 3 | CAETHG_0233, | 4-imidazolone-5-propanoate amidohydrolase (H2O + 4- | 0 | 0.05214 |
| | LMOMA | | CAETHG_3359, | Imidazolone-5-propanoate --> N-Formimino-L-glutamate), | | |
| | | | CAETHG_3510 | ATP:acetate phosphotransferase (ATP + H+ + Propionate --> ADP + | | |
| | | | | Propionyl phosphate), 2,6-Diaminoheptanedioate:2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 13 | Yield - | 3 | CAETHG_0234, | 4,5-Dihydro-4-oxo-5-imidazolepropanoate hydro-lyase (4- | 0 | 0.05214 |
| | LMOMA | | CAETHG_3359, | Imidazolone-5-propanoate <=> H2O + Urocanate), ATP:acetate | | |
| | | | CAETHG_3510 | phosphotransferase (ATP + H+ + Propionate --> ADP + Propionyl | | |
| | | | | phosphate), 2,6-Diaminoheptanedioate:2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 14 | Yield - | 2 | CAETHG_3359, | ATP:acetate phosphotransferase (ATP + H+ + Propionate --> ADP + | 0 | 0.052041 |
| | LMOMA | | CAETHG_3510 | Propionyl phosphate), 2,6-Diaminoheptanedioate:2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 15 | Yield - | 2 | CAETHG_3358, | Phosphate transacetylase (Phosphate + Acetyl-CoA + H+ <=> CoA + | 0 | 0.052041 |
| | LMOMA | | CAETHG_3510 | Acetylphosphate), 2,6-Diaminoheptanedioate:2-oxoglutarate | | |
| | | | | aminotransferase (H2O + 1-Glutamate + H+ + | | |
| | | | | tetrahydrodipicolinate <-- 2-Oxoglutarate + LL-2,6- | | |
| | | | | Diaminopimelate) | | |
| 16 | Yield - | 3 | CAETHG_0160, | N-Ribosylnicotinamide:orthophosphate ribosyltransferase | 0 | 0.051891 |
| | LMOMA | | CAETHG_3359, | (Phosphate + N-Ribosylnicotinamide <=> Nicotinamide + Ribose 1- | | |
| | | | CAETHG 0909 | phosphate), ATP:acetate phosphotransferase (ATP + H+ + | | |
| | | | | Propionate --> ADP + Propionyl phosphate), Prephenate:NAD + | | |
| | | | | oxidoreductase(decarboxylating) (NAD + Prephenate --> NADH + | | |
| | | | | CO2 + p-hydroxyphenylpyruvate) | | |
| 17 | Yield - | 3 | CAETHG_0160, | N-Ribosylnicotinamide:orthophosphate ribosyltransferase | 0 | 0.051678 |
| | LMOMA | | CAETHG_3359, | (Phosphate + N-Ribosylnicotinamide <=> Nicotinamide + Ribose 1- | | |
| | | | CAETHG_0498 | phosphate), ATP:acetate phosphotransferase (ATP + H+ + | | |
| | | | | Propionate --> ADP + Propionyl phosphate), Cystathionine beta | | |
| | | | | lyase (H2O + Cystathionine --> NH3 + Pyruvate + Homocysteine) | | |
| 18 | Yield - | 3 | CAETHG_1371, | Adenosine 5′-monophosphate phosphohydrolase (H2O + AMP <=> | 0 | 0.051609 |
| | LMOMA | | CAETHG_2753, | Phosphate + H+ + Adenosine), Isocitrate dehydrogenase (NAD + | | |
| | | | CAETHG_3359 | Isocitrate <=> NADH + CO2 + 2-Oxoglutarate + H+), ATP:acetate | | |
| | | | | phosphotransferase (ATP + H+ + Propionate --> ADP + Propionyl | | |
| | | | | phosphate) | | |
| 19 | Yield - | 3 | CAETHG_0160, | N-Ribosylnicotinamide:orthophosphate ribosyltransferase | 0 | 0.05157 |
| | LMOMA | | CAETHG_3021, | (Phosphate + N-Ribosylnicotinamide <=> Nicotinamide + Ribose 1- | | |
| | | | CAETHG_3359 | phosphate), L-Arginine iminohydrolase (H2O + L-Arginine <=> | | |
| | | | | NH3 + Citrulline), ATP:acetate phosphotransferase (ATP + H+ + | | |
| | | | | Propionate --> ADP + Propionyl phosphate) | | |
| 20 | Yield - | 2 | CAETHG_0160, | N-Ribosylnicotinamide:orthophosphate ribosyltransferase | 0 | 0.051567 |
| | LMOMA | | CAETHG_3359 | (Phosphate + N-Ribosylnicotinamide <=> Nicotinamide + Ribose 1- | | |
| | | | | phosphate), ATP:acetate phosphotransferase (ATP + H+ + | | |
| | | | | Propionate --> ADP + Propionyl phosphate) | | |
| 21 | Yield - | 2 | CAETHG_3358, | Phosphate transacetylase (Phosphate + Acetyl-CoA + H+ <=> CoA + | 0 | 0.051567 |
| | LMOMA | | CAETHG_3924 | Acetylphosphate), D-Ribose 1,5-phosphomutase (Ribose 1- | | |
| | | | | phosphate <=> ribose-S-phosphate) | | |
| 22 | Yield - | 2 | CAETHG_1371, | Adenosine 5′-monophosphate phosphohydrolase (H2O + AMP <=> | 0 | 0.051567 |
| | LMOMA | | CAETHG_3359 | Phosphate + H+ + Adenosine), ATP:acetate phosphotransferase | | |
| | | | | (ATP + H+ + Propionate --> ADP + Propionyl phosphate) | | |
| 23 | Yield - | 2 | CAETHG_3359, | ATP:acetate phosphotransferase (ATP + H+ + Propionate --> ADP + | 0 | 0.051567 |
| | LMOMA | | CAETHG 3924 | Propionyl phosphate), D-Ribose 1,5-phosphomutase (Ribose 1- | | |
| | | | | phosphate <=> ribose-5-phosphate) | | |
| 24 | Yield - | 2 | CAETHG_1270, | AMP:pyrophosphate phosphoribosyltransferase (PPi + AMP <=> | 0 | 0.051561 |
| | LMOMA | | CAETHG_3359 | PRPP + Adenine), ATP:acetate phosphotransferase (ATP + H+ + | | |
| | | | | Propionate --> ADP + Propionyl phosphate) | | |
| 25 | Yield - | 2 | CAETHG_3359, | ATP:acetate phosphotransferase (ATP + H+ + Propionate --> ADP + | 0 | 0.048294 |
| | LMOMA | | CAETHG_0686 | Propionyl phosphate), L-Threonine acetaldehyde-lyase (L- | | |
| | | | | Threonine --> Glycine + Acetaldehyde) | | |
| 26 | Yield - | 2 | CAETHG_3359, | ATP:acetate phosphotransferase (ATP + H+ + Propionate --> ADP + | 0 | 0.046551 |
| | LMOMA | | CAETHG_0498 | Propionyl phosphate), Cystathionine beta lyase (H2O + | | |
| | | | | Cystathionine --> NH3 + Pyruvate + Homocysteine) | | |
| 27 | Yield - | 2 | CAETHG_3293, | Cysteine desulfhydrase (H2O + L-Cysteine <=> NH3 + Pyruvate + | 0 | 0.045591 |
| | LMOMA | | CAETHG_3359 | H2S), ATP:acetate phosphotransferase (ATP + H+ + Propionate --> | | |
| | | | | ADP + Propionyl phosphate) | | |
| 28 | Yield - | 2 | CAETHG_3299, | 2-Deoxy-D-ribose-5-phosphate acetaldehyde-lyase (deoxyribose-5- | 0 | 0.045288 |
| | LMOMA | | CAETHG 3359 | phosphate <=> Acetaldehyde + Glyceraldehyde3-phosphate), | | |
| | | | | ATP:acetate phosphotransferase (ATP + H+ + Propionate --> ADP + | | |
| | | | | Propionyl phosphate) | | |
| 29 | Yield - | 2 | CAETHG_2475, | dGTP triphosphohydrolase (H2O + dGTP --> H+ + Deoxyguanosine + | 0 | 0.045285 |
| | LMOMA | | CAETHG_3359 | Triphosphate), ATP:acetate phosphotransferase (ATP + H+ + | | |
| | | | | Propionate --> ADP + Propionyl phosphate) | | |
| 30 | Yield - | 2 | CAETHG_3021, | L-Arginine iminohydrolase (H2O + L-Arginine <=> NH3 + Citrulline), | 0 | 0.044994 |
| | LMOMA | | CAETHG_3359 | ATP:acetate phosphotransferase (ATP + H+ + Propionate --> ADP + | | |
| | | | | Propionyl phosphate) | | |
| 31 | Yield - | 2 | CAETHG_3164, | UMP:pyrophosphate phosphoribosyltransferase (Uracil + PRPP --> | 0 | 0.044943 |
| | LMOMA | | CAETHG_3359 | PPi + UMP), ATP:acetate phosphotransferase (ATP + H+ + | | |
| | | | | Propionate --> ADP + Propionyl phosphate) | | |
| 32 | Yield - | 1 | CAETHG_3359 | ATP:acetate phosphotransferase (ATP + H+ + Propionate --> ADP + | 0 | 0.04494 |
| | LMOMA | | | Propionyl phosphate) | | |
| 33 | Yield - | 1 | CAETHG_3358 | Phosphate transacetylase (Phosphate + Acetyl-CoA + H+ <=> CoA + | 0 | 0.04494 |
| | LMOMA | | | Acetylphosphate) | | |
| 34 | Yield - | 2 | CAETHG_0234, | 4,5-Dihydro-4-oxo-5-imidazolepropanoate hydro-lyase (4- | 0 | 0.020286 |
| | LMOMA | | CAETHG_2751 | Imidazolone-5-propanoate <=> H2O + Urocanate), Citramalate | | |
| | | | | synthase (H2O + Pyruvate + Acetyl-CoA <=> CoA + Citramalate) | | |
| 35 | Yield - | 1 | CAETHG_2751 | Citramalate synthase (H2O + Pyruvate + Acetyl-CoA <=> CoA + | 0 | 0.020283 |
| | LMOMA | | | Citramalate) | | |
| 36 | Yield - | 1 | CAETHG_1371 | Adenosine 5′-monophosphate phosphohydrolase (H2O + AMP <=> | 0 | 0.020094 |
| | LMOMA | | | Phosphate + H+ + Adenosine) | | |
| 37 | Yield - | 1 | CAETHG 1270 | AMP:pyrophosphate phosphoribosyltransferase (PPi + AMP <=> | 0 | 0.020094 |
| | LMOMA | | | PRPP + Adenine) | | |
| 38 | Yield - | 1 | CAETHG_3924 | D-Ribose 1,5-phosphomutase (Ribose 1-phosphate <=> ribose-5- | 0 | 0.020094 |
| | LMOMA | | | phosphate) | | |

### Example 5

This example describes disruptions for improved production of isopropanol in Wood-Ljungdahl microorganisms. Metabolic modeling experiments were performed as described in Example 1. Production of isopropanol in Wood-Ljungdahl microorganisms is described, e.g., in WO 2012/115527. The following pathway was used to model isopropanol production herein: 2.0 Acetyl-CoA-->CoA+Acetoacetyl-CoA; Acetate+Acetoacetyl-CoA-->Acetyl-CoA+Acetoacetate; Acetoacetate-->CO₂+Acetone; Isopropanol-->Isopropanol_ext.

| | | # | | | FVA | |
|---|---|---|---|---|---|---|
| | | Disrupted | Disrupted | Disrupted | minimum | Fitness |
| # | Technique | genes | genes | reactions | yield | score |
| 1 | Yield - | 6 | CAETHG_2753, | Isocitrate dehydrogenase (NAD + Isocitrate <=> NADH + CO2 + 2- | 0 | 0.04386 |
| | LMOMA | | CAETHG_2932, | Oxoglutarate + H+), Alpha-acetolactate decarboxylase (ALCTT --> | | |
| | | | CAETHG_3021, | CO2 + (R)-Acetoin), L-Arginine iminohydrolase (H2O + L- | | |
| | | | CAETHG_3358, | Arginine <=> NH3 + Citrulline), Phosphate transacetylase | | |
| | | | CAETHG_0498, | (Phosphate + Acetyl-CoA + H+ <=> CoA + Acetylphosphate), | | |
| | | | CAETHG_0686 | Cystathionine beta lyase (H2O + Cystathionine --> NH3 + | | |
| | | | | Pyruvate + Homocysteine), L-Threonine acetaldehyde-lyase | | |
| | | | | (L-Threonine --> Glycine + Acetaldehyde) | | |
| 2 | Yield - | 5 | CAETHG_2753, | Isocitrate dehydrogenase (NAD + Isocitrate <=> NADH + CO2 + 2- | 0 | 0.043854 |
| | LMOMA | | CAETHG_2932, | Oxoglutarate + H+), Alpha-acetolactate decarboxylase (ALCTT --> | | |
| | | | CAETHG_3358, | CO2 + (R)-Acetoin), Phosphate transacetylase (Phosphate + Acetyl- | | |
| | | | CAETHG_0498, | CoA + H+ <=> CoA + Acetylphosphate), Cystathionine beta lyase | | |
| | | | CAETHG_0686 | (H2O + Cystathionine --> NH3 + Pyruvate + Homocysteine), L- | | |
| | | | | Threonine acetaldehyde-lyase (L-Threonine --> Glycine + | | |
| | | | | Acetaldehyde) | | |
| 3 | Yield - | 6 | CAETHG_2932, | Alpha-acetolactate decarboxylase (ALCTT --> CO2 + (R)-Acetoin), L- | 0 | 0.043791 |
| | LMOMA | | CAETHG_3021, | Arginine iminohydrolase (H2O + L-Arginine <=> NH3 + Citrulline), | | |
| | | | CAETHG_3358, | Phosphate transacetylase (Phosphate + Acetyl-CoA + H+ <=> CoA + | | |
| | | | CAETHG_3510, | Acetylphosphate), 2,6-Diaminoheptanedioate:2-oxoglutarate | | |
| | | | CAETHG_0498, | aminotransferase (H2O + L-Glutamate + H+ + | | |
| | | | CAETHG_0686 | tetrahydrodipicolinate <-- 2-Oxoglutarate + LL-2,6- | | |
| | | | | Diaminopimelate), Cystathionine beta lyase (H2O + Cystathionine --> | | |
| | | | | NHB + Pyruvate + Homocysteine), L-Threonine acetaldehyde- | | |
| | | | | lyase (L-Threonine --> Glycine + Acetaldehyde) | | |
| 4 | Yield - | 5 | CAETHG_2932, | Alpha-acetolactate decarboxylase (ALCTT --> CO2 + (R)-Acetoin), | 0 | 0.043782 |
| | LMOMA | | CAETHG_3358, | Phosphate transacetylase (Phosphate + Acetyl-CoA + H+ <=> CoA + | | |
| | | | CAETHG_3510, | Acetylphosphate), 2,6-Diaminoheptanedioate:2-oxoglutarate | | |
| | | | CAETHG_0498, | aminotransferase (H2O + L-Glutamate + H+ + | | |
| | | | CAETHG_0686 | tetrahydrodipicolinate <-- 2-Oxoglutarate + LL-2,6- | | |
| | | | | Diaminopimelate), Cystathionine beta lyase (H2O + Cystathionine --> | | |
| | | | | NH3 + Pyruvate + Homocysteine), L-Threonine acetaldehyde- | | |
| | | | | lyase (L-Threonine --> Glycine + Acetaldehyde) | | |
| 5 | Yield - | 5 | CAETHG_2932, | Alpha-acetolactate decarboxylase (ALCTT --> CO2 + (R)-Acetoin), L- | 0 | 0.04377 |
| | LMOMA | | CAETHG_3021, | Arginine iminohydrolase (H2O + L-Arginine <=> NH3 + Citrulline), | | |
| | | | CAETHG_3358, | Phosphate transacetylase (Phosphate + Acetyl-CoA + H+ <=> CoA + | | |
| | | | CAETHG_0498, | Acetylphosphate), Cystathionine beta lyase (H2O + Cystathionine --> | | |
| | | | CAETHG_0686 | NH3 + Pyruvate + Homocysteine), L-Threonine acetaldehyde- | | |
| | | | | lyase (L-Threonine --> Glycine + Acetaldehyde) | | |
| 6 | Yield - | 4 | CAETHG_2932, | Alpha-acetolactate decarboxylase {ALCTT --> CO2 + (R)-Acetoin), | 0 | 0.043758 |
| | LMOMA | | CAETHG_3359, | ATP:acetate phosphotransferase (ATP + H+ + Propionate --> ADP + | | |
| | | | CAETHG_0498, | Propionyl phosphate), Cystathionine beta lyase (H2O + | | |
| | | | CAETHG_0686 | Cystathionine --> NH3 + Pyruvate + Homocysteine), L-Threonine | | |
| | | | | acetaldehyde-lyase (L-Threonine --> Glycine + Acetaldehyde) | | |
| 7 | Yield - | 4 | CAETHG_2932, | Alpha-acetolactate decarboxylase (ALCTT --> CO2 + (R)-Acetoin), | 0 | 0.043758 |
| | LMOMA | | CAETHG_3358, | Phosphate transacetylase (Phosphate + Acetyl-CoA + H+ <=> CoA + | | |
| | | | CAETHG_0498, | Acetylphosphate), Cystathionine beta lyase (H2O + Cystathionine --> | | |
| | | | CAETHG_0686 | NH3 + Pyruvate + Homocysteine), L-Threonine acetaldehyde- | | |
| | | | | lyase (L-Threonine --> Glycine + Acetaldehyde) | | |
| 8 | Yield - | 3 | CAETHG_2932, | Alpha-acetolactate decarboxylase (ALCTT --> CO2 + (R)-Acetoin), | 0 | 0.043644 |
| | LMOMA | | CAETHG_3368, | Phosphate transacetylase (Phosphate + Acetyl-CoA + H + <=> CoA + | | |
| | | | CAETHG 0686 | Acetylphosphate), L-Threonine acetaldehyde-lyase (L-Threonine --> | | |
| | | | | Glycine + Acetaldehyde) | | |
| 9 | Yield - | 3 | CAETHG_2932, | Alpha-acetolactate decarboxylase (ALCTT --> CO2 + (R)-Acetoin), | 0 | 0.043644 |
| | LMOMA | | CAETHG_3359, | ATP:acetate phosphotransferase (ATP + H+ + Propionate --> ADP + | | |
| | | | CAETHG_0686 | Propionyl phosphate), L-Threonine acetaldehyde-lyase (L- | | |
| | | | | Threonine --> Glycine + Acetaldehyde) | | |
| 10 | Yield - | 4 | CAETHG_2932, | Alpha-acetolactate decarboxylase (ALCTT --> CO2 + (R)-Acetoin), L- | 0 | 0.043107 |
| | LMOMA | | CAETHG_3021, | Arginine iminohydrolase (H2O + L-Arginine <=> NH3 + Citrulline), | | |
| | | | CAETHG_3359, | ATP:acetate phosphotransferase (ATP + H+ + Propionate --> ADP + | | |
| | | | CAETHG_0498 | Propionyl phosphate), Cystathionine beta lyase (H2O + | | |
| | | | | Cystathionine --> NH3 + Pyruvate + Homocysteine) | | |
| 11 | Yield - | 3 | CAETHG_2932, | Alpha-acetolactate decarboxylase (ALCTT --> CO2 + (R)-Acetoin), | 0 | 0.043095 |
| | LMOMA | | CAETHG_3358, | Phosphate transacetylase (Phosphate + Acetyl-CoA + H+ <=> CoA + | | |
| | | | CAETHG_0498 | Acetylphosphate), Cystathionine beta lyase (H2O + Cystathionine --> | | |
| | | | | NH3 + Pyruvate + Homocysteine) | | |
| 12 | Yield - | 3 | CAETHG_2932, | Alpha-acetolactate decarboxylase (ALCTT --> CO2 + (R)-Acetoin), | 0 | 0.043095 |
| | LMOMA | | CAETHG_3359, | ATP:acetate phosphotransferase (ATP + H+ + Propionate --> ADP + | | |
| | | | CAETHG_0498 | Propionyl phosphate), Cystathionine beta lyase (H2O + | | |
| | | | | Cystathionine --> NH3 + Pyruvate + Homocysteine) | | |
| 13 | Yield - | 3 | CAETHG_1371, | Adenosine 5′-monophosphate phosphohydrolase (H2O + AMP <=> | 0 | 0.04308 |
| | LMOMA | | CAETHG_2932, | Phosphate + H+ + Adenosine), Alpha-acetolactate decarboxylase | | |
| | | | CAETHG_3359 | (ALCTT --> CO2 + (R)-Acetoin), ATP:acetate phosphotransferase | | |
| | | | | (ATP + H+ + Propionate --> ADP + Propionyl phosphate) | | |
| 14 | Yield - | 3 | CAETHG_0160, | N-Ribosylnicotinamide:orthophosphate ribosyltransferase | 0 | 0.04308 |
| | LMOMA | | CAETHG_2932, | (Phosphate + N-Ribosylnicotinamide <=> Nicotinamide + Ribose 1- | | |
| | | | CAETHG_3359 | phosphate), Alpha-acetolactate decarboxylase (ALCTT --> CO2 + | | |
| | | | | (R)-Acetoin), ATP:acetate phosphotransferase (ATP + H+ + | | |
| | | | | Propionate - > ADP + Propionyl phosphate) | | |
| 15 | Yield - | 3 | CAETHG 2932, | Alpha-acetolactate decarboxylase (ALCTT --> CO2 + (R)-Acetoin), | 0 | 0.043029 |
| | LMOMA | | CAETHG_3021, | L-Arginine iminohydrolase (H2O + L-Arginine <=> NH3 + | | |
| | | | CAETHG_3359 | Citrulline), ATPracetate phosphotransferase (ATP + H+ + | | |
| | | | | Propionate --> ADP + Propionyl phosphate) | | |
| 16 | Yield - | 3 | 0, | Acetyl-CoA:carbon-dioxide ligase (ADP-forming) (ATP + Acetyl- | 0 | 0.043023 |
| | LMOMA | | CAETHG_2932, | CoA + H2CO3 --> ADP + Phosphate + H+ + Malonyl-CoA), Alpha- | | |
| | | | CAETHG_3359 | acetolactate decarboxylase (ALCTT --> CO2 + (R)-Acetoin), | | |
| | | | | ATP:acetate phosphotransferase (ATP + H+ + Propionate --> ADP + | | |
| | | | | Propionyl phosphate) | | |
| 17 | Yield - | 2 | CAETHG_2932, | Alpha-acetolactate decarboxylase (ALCTT --> CO2 + (R)-Acetoin), | 0 | 0.043017 |
| | LMOMA | | CAETHG_3359 | ATP:acetate phosphotransferase (ATP + H+ + Propionate --> ADP + | | |
| | | | | Propionyl phosphate) | | |
| 18 | Yield - | 2 | CAETHG_2932, | Alpha-acetolactate decarboxylase (ALCTT --> CO2 + (R)-Acetoin), | 0 | 0.043017 |
| | LMOMA | | CAETHG_3358 | Phosphate transacetylase (Phosphate + Acetyl-CoA + H+ <=> CoA + | | |
| | | | | Acetylphosphate) | | |
| 19 | Yield - | 3 | CAETHG_3359, | ATP:acetate phosphotransferase (ATP + H+ + Propionate --> ADP + | 0 | 0.037755 |
| | LMOMA | | CAETHG_0498, | Propionyl phosphate), Cystathionine beta lyase (H2O + | | |
| | | | CAETHG_0686 | Cystathionine --> NH3 + Pyruvate + Homocysteine), L-Threonine | | |
| | | | | acetaldehyde-lyase (L-Threonine --> Glycine + Acetaldehyde) | | |
| 20 | Yield - | 3 | CAETHG_3021, | L-Arginine iminohydrolase (H2O + L-Arginine <=> NH3 + Citrulline), | 0 | 0.037077 |
| | LMOMA | | CAETHG_3359, | ATP:acetate phosphotransferase (ATP+ H+ + Propionate --> ADP + | | |
| | | | CAETHG_0686 | Propionyl phosphate), L-Threonine acetaldehyde-lyase (L- | | |
| | | | | Threonine --> Glycine + Acetaldehyde) | | |
| 21 | Yield - | 2 | CAETHG_3358, | Phosphate transacetylase (Phosphate + Acetyl-CoA + H+ <=> CoA + | 0 | 0.037038 |
| | IMOMA | | CAETHG_0686 | Acetylphosphate), L-Threonine acetaldehyde-lyase (L-Threonine --> | | |
| | | | | Glycine + Acetaldehyde) | | |
| 22 | Yield - | 2 | CAETHG_3359, | ATP:acetate phosphotransferase (ATP + H+ + Propionate --> ADP + | 0 | 0.037038 |
| | LMOMA | | CAETHG_0686 | Propionyl phosphate), L-Threonine acetaldehyde-lyase (L- | | |
| | | | | Threonine --> Glycine + Acetaldehyde) | | |
| 23 | Yield - | 3 | CAETHG_2441, | Pyruvate kinase (ADP + Phosphoenolpyruvate --> ATP + Pyruvate), | 0 | 0.036639 |
| | LMOMA | | CAETHG_3293, | Cysteine desulfhydrase (H2O + L-Cysteine <=> NH3 + Pyruvate + | | |
| | | | CAETHG_3359 | H2S), ATP:acetate phosphotransferase (ATP + H+ + Propionate --> | | |
| | | | | ADP + Propionyl phosphate) | | |
| 24 | Yield - | 3 | CAETHG_3293, | Cysteine desulfhydrase (H2O + L-Cysteine <=> NH3 + Pyruvate + | 0 | 0.036468 |
| | LMOMA | | CAETHG_3359, | H2S), ATP:acetate phosphotransferase (ATP + H+ + Propionate --> | | |
| | | | CAETHG_0498 | ADP + Propionyl phosphate), Cystathionine beta lyase (H2O + | | |
| | | | | Cystathionine --> NH3 + Pyruvate + Homocysteine) | | |
| 25 | Yield - | 2 | CAETHG_3293, | Cysteine desulfhydrase (H2O + L-Cysteine <=> NH3 + Pyruvate + | 0 | 0.035355 |
| | LMOMA | | CAETHG_3359 | H2S), ATP:acetate phosphotransferase (ATP + H+ + Propionate --> | | |
| | | | | ADP + Propionyl phosphate) | | |
| 26 | Yield - | 2 | CAETHG_0234, | 4,5-Dihydro-4-oxo-5-imidazolepropanoate hydro-lyase (4- | 0 | 0.031752 |
| | LMOMA | | CAETHG_3359 | Imidazolone-5-propanoate <=> H2O + Urocanate), ATP:acetate | | |
| | | | | phosphotransferase (ATP + H+ + Propionate --> ADP + Propionyl | | |
| | | | | phosphate) | | |
| 27 | Yield - | 2 | CAETHG_0233, | 4-imidazolone-5-propanoate amidohydrolase (H2O + 4- | 0 | 0.031752 |
| | LMOMA | | CAETHG_3359 | Imidazolone-5-propanoate --> N-Formimino-L-glutamate), | | |
| | | | | ATP:acetate phosphotransferase (ATP + H+ + Propionate --> ADP + | | |
| | | | | Propionyl phosphate) | | |
| 28 | Yield - | 2 | CAETHG_3021, | L-Arginine iminohydrolase (H2O + L-Arginine <=> NH3 + Citrulline), | 0 | 0.031737 |
| | LMOMA | | CAETHG_3359 | ATP:acetate phosphotransferase (ATP + H+ + Propionate --> ADP + | | |
| | | | | Propionyl phosphate) | | |
| 29 | Yield - | 2 | CAETHG_2475, | dGTP triphosphohydrolase (H2O + dGTP --> H+ + Deoxyguanosine + | 0 | 0.031731 |
| | LMOMA | | CAETHG_3359 | Triphosphate), ATP:acetate phosphotransferase (ATP + H+ + | | |
| | | | | Propionate --> ADP + Propionyl phosphate) | | |
| 30 | Yield - | 2 | CAETHG_2475, | dGTP triphosphohydrolase (H2O + dGTP --> H+ + Deoxyguanosine + | 0 | 0.031731 |
| | LMOMA | | CAETHG_3358 | Triphosphate), Phosphate transacetylase (Phosphate + Acetyl-CoA + | | |
| | | | | H+ <=> CoA + Acetylphosphate) | | |
| 31 | Yield - | 1 | CAETHG_3358 | Phosphate transacetylase (Phosphate + Acetyl-CoA + H+ <=> CoA + | 0 | 0.031707 |
| | LMOMA | | | Acetylphosphate) | | |
| 32 | Yield - | 1 | CAETHG_3359 | ATP:acetate phosphotransferase (ATP + H+ + Propionate --> ADP + | 0 | 0.031707 |
| | LMOMA | | | Propionyl phosphate) | | |

### Example 6

This example describes disruptions for improved production of lactate in Wood-Ljungdahl microorganisms. Metabolic modeling experiments were performed as described in Example 1. Lactate is a native product of Wood-Ljungdahl microorganisms.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | BPCY- | 4 | CAETHG_2753, | Isocitrate dehydrogenase (NAD + Isocitrate <=> NADH + CO2 + 2- | 0 | 0.011882 |
| | FBA | | CAETHG_2909, | Oxoglutarate + H+), ATP:pyruvate, orthophosphate | | |
| | | | CAETHG_3293, | phosphotransferase (ATP + Phosphate + Pyruvate + H+ --> PPi + | | |
| | | | CAETHG_3358 | AMP + Phosphoenolpyruvate), Cysteine desulfhydrase (H2O + L- | | |
| | | | | Cysteine <=> NH3 + Pyruvate + H2S), Phosphate transacetylase | | |
| | | | | (Phosphate + Acetyl-CoA + H+ <=> CoA + Acetylphosphate) | | |
| 2 | BPCY- | 4 | CAETHG_2753, | Isocitrate dehydrogenase (NAD + Isocitrate <=> NADH + CO2 + 2- | 0 | 0.011882 |
| | FBA | | CAETHG_2909, | Oxoglutarate + H+), ATP:pyruvate, orthophosphate | | |
| | | | CAETHG_3293, | phosphotransferase (ATP + Phosphate + Pyruvate + H+ --> PPi + | | |
| | | | CAETHG_3359 | AMP + Phosphoenolpyruvate), Cysteine desulfhydrase (H2O + L- | | |
| | | | | Cysteine <=> NH3 + Pyruvate + H2S), ATP:acetate | | |
| | | | | phosphotransferase (ATP + H+ + Propionate --> ADP + Propionyl | | |
| | | | | phosphate) | | |
| 3 | BPCY - | 4 | CAETHG_2751, | Citramalate synthase (H2O + Pyruvate + Acetyl-CoA <=> CoA + | 0 | 0.011873 |
| | FBA | | CAETHG_2753, | Citramalate), Isocitrate dehydrogenase (NAD + Isocitrate <=> | | |
| | | | CAETHG_2909, | NADH + CO2 + 2-Oxoglutarate + H+), ATP:pyruvate, orthophosphate | | |
| | | | CAETHG_3358 | phosphotransferase (ATP + Phosphate + Pyruvate + H+ --> PPi + | | |
| | | | | AMP + Phosphoenolpyruvate), Phosphate transacetylase | | |
| | | | | (Phosphate + Acetyl-CoA + H+ <=> CoA + Acetylphosphate) | | |
| 4 | BPCY - | 4 | CAETHG_2753, | Isocitrate dehydrogenase (NAD + Isocitrate <=> NADH + CO2 + 2- | 0 | 0.011786 |
| | F8A | | CAETHG_2909, | Oxoglutarate + H+), ATP:pyruvate, orthophosphate | | |
| | | | CAETHG_3358, | phosphotransferase (ATP + Phosphate + Pyruvate + H+ --> PPi + | | |
| | | | CAETHG_0498 | AMP + Phosphoenolpyruvate), Phosphate transacetylase | | |
| | | | | (Phosphate + Acetyl-CoA + H+ <=> CoA + Acetylphosphate), | | |
| | | | | Cystathionine beta lyase (H2O + Cystathionine --> NH3 + Pyruvate + | | |
| | | | | Homocysteine) | | |
| 5 | BPCY- | 3 | CAETHG_2753, | Isocitrate dehydrogenase (NAD + Isocitrate <=> NADH + CO2 + 2- | 0 | 0.011696 |
| | FBA | | CAETHG_2909, | Oxoglutarate + H+), ATP:pyruvate, orthophosphate | | |
| | | | CAETHG_3358 | phosphotransferase (ATP + Phosphate + Pyruvate + H+ --> PPi + | | |
| | | | | AMP + Phosphoenolpyruvate), Phosphate transacetylase | | |
| | | | | (Phosphate + Acetyl-CoA + H+ <=> CoA + Acetylphosphate) | | |
| 6 | BPCY- | 3 | CAETHG_2751, | Citramalate synthase (H2O + Pyruvate + Acetyl-CoA <=> CoA + | 0 | 0.010722 |
| | FBA | | CAETHG_2909, | Citramalate), ATP:pyruvate, orthophosphate phosphotransferase | | |
| | | | CAETHG_3358 | (ATP + Phosphate + Pyruvate + H+ --> PPi + AMP + | | |
| | | | | Phosphoenolpyruvate), Phosphate transacetylase (Phosphate + | | |
| | | | | Acetyl-CoA + H+ <=> CoA + Acetylphosphate) | | |
| 7 | BPCY- | 2 | CAETHG_2909, | ATP:pyruvate, orthophosphate phosphotransferase (ATP + | 0 | 0.010534 |
| | FBA | | CAETHG_3359 | Phosphate + Pyruvate + H+ --> PPi + AMP + Phosphoenolpyruvate), | | |
| | | | | ATP:acetate phosphotransferase (ATP + H+ + Propionate --> ADP + | | |
| | | | | Propionyl phosphate) | | |
| 8 | BPCY - | 2 | CAETHG_2909, | ATP:pyruvate, orthophosphate phosphotransferase (ATP + | 0 | 0.010534 |
| | FBA | | CAETHG_3358 | Phosphate + Pyruvate + H+ --> PPi + AMP + Phosphoenolpyruvate), | | |
| | | | | Phosphate transacetylase (Phosphate + Acetyl-CoA + H+ <=> CoA + | | |
| | | | | Acetylphosphate) | | |
| 9 | BPCY- | 2 | CAETHG_2909, | ATP:pyruvate, orthophosphate phosphotransferase (ATP + | 0 | 0.010387 |
| | FBA | | CAETHG_3358 | Phosphate + Pyruvate + H+ --> PPi + AMP + Phosphoenolpyruvate), | | |
| | | | | Phosphate transacetylase (Phosphate + Acetyl-CoA + H+ <=> CoA + | | |
| | | | | Acetylphosphate) | | |
| 10 | BPCY- | 3 | CAETHG_2753, | Isocitrate dehydrogenase (NAD + Isocitrate <=> NADH + CO2 + 2- | 0 | 0.010038 |
| | FBA | | CAETHG_3293, | Oxoglutarate + H+), Cysteine desulfhydrase (H2O + L-Cysteine <=> | | |
| | | | CAETHG_3358 | NH3 + Pyruvate + H2S), Phosphate transacetylase (Phosphate + | | |
| | | | | Acetyl-CoA + H+ <=> CoA + Acetylphosphate) | | |
| 11 | BPCY- | 3 | CAETHG_2751, | Citramalate synthase (H2O + Pyruvate + Acetyl-CoA <=> CoA + | 0 | 0.00988 |
| | FBA | | CAETHG_2753, | Citramalate), Isocitrate dehydrogenase (NAD + Isocitrate <=> | | |
| | | | CAETHG_3358 | NADH + CO2 + 2-Oxoglutarate + H+), Phosphate transacetylase | | |
| | | | | (Phosphate + Acetyl-CoA + H+ <=> CoA + Acetylphosphate) | | |
| 12 | BPCY - | 2 | CAETHG_2753, | Isocitrate dehydrogenase (NAD + Isocitrate <=> NADH + CO2 + 2- | 0 | 0.009678 |
| | FBA | | CAETHG_3358 | Oxoglutarate + H+), Phosphate transacetylase (Phosphate + Acetyl- | | |
| | | | | CoA + H+ <=> CoA + Acetylphosphate) | | |
| 13 | BPCY - | 2 | CAETHG_2753, | Isocitrate dehydrogenase (NAD + Isocitrate <=> NADH + CO2 + 2- | 0 | 0.009586 |
| | FBA | | CAETHG_3358 | Oxoglutarate + H+), Phosphate transacetylase (Phosphate + Acetyl- | | |
| | | | | CoA + H+ <=> CoA + Acetylphosphate) | | |
| 14 | BPCY- | 2 | CAETHG_2751, | Citramalate synthase (H2O + Pyruvate + Acetyl-CoA <=> CoA + | 0 | 0.008686 |
| | FBA | | CAETHG_3359 | Citramalate), ATP:acetate phosphotransferase (ATP + H+ + | | |
| | | | | Propionate --> ADP + Propionyl phosphate) | | |
| 15 | BPCY - | 2 | CAETHG_2751, | Citramalate synthase (H2O + Pyruvate + Acetyl-CoA <=> CoA + | 0 | 0.008686 |
| | FBA | | CAETHG_3358 | Citramalate), Phosphate transacetylase (Phosphate + Acetyl-CoA + | | |
| | | | | H+ <=> CoA + Acetylphosphate) | | |
| 16 | BPCY - | 1 | CAETHG_3359 | ATP:acetate phosphotransferase (ATP + H+ + Propionate --> ADP + | 0 | 0.008474 |
| | FBA | | | Propionyl phosphate) | | |
| 17 | BPCY- | 1 | CAETHG_3358 | Phosphate transacetylase (Phosphate + Acetyl-CoA + H+ <=> CoA + | 0 | 0.008474 |
| | FBA | | | Acetylphosphate) | | |
| 18 | BPCY- | 2 | CAETHG_2753, | Isocitrate dehydrogenase (NAD + Isocitrate <=> NADH + CO2 + 2- | 0 | 0.001316 |
| | FBA | | CAETHG_2909 | Oxoglutarate + H+), ATP:pyruvate, orthophosphate | | |
| | | | | phosphotransferase (ATP + Phosphate + Pyruvate + H+ --> PPi + | | |
| | | | | AMP + Phosphoenolpyruvate) | | |
| 19 | Yield - | 5 | CAETHG_2210, | NADP-dependent electron-bifurcating [FeFe]-hydrogenase (Hyt) | 0 | 0.036108 |
| | LMOMA | | CAETHG_2224, | (NADP + Oxidizedferredoxin + 2.0H2 <=> NADPFI + 3.0H+ + | | |
| | | | CAETHG_2798, | Reducedferredoxin), Alpha-acetolactate decarboxylase (ALCTT --> | | |
| | | | CAETHG_2932, | CO2 + (R)-Acetoin), 2-Deoxy-D-ribose-5-phosphate acetaldehyde- | | |
| | | | CAETHG_3299 | lyase (deoxyribose-5-phosphate <=> Acetaldehyde + | | |
| | | | | Glyceraldehyde3-phosphate) | | |
| 20 | Yield - | 5 | CAETHG_2210, | NADP-dependent electron-bifurcating [FeFe]-hydrogenase (Hyt) | 0 | 0.035997 |
| | LMOMA | | CAETHG_2224, | (NADP + Oxidizedferredoxin + 2.0H2 <=> NADPH + 3.0H+ + | | |
| | | | CAETHG_2798, | Reducedferredoxin), Alpha-acetolactate decarboxylase (ALCTT --> | | |
| | | | CAETHG_29B2, | CO2 + (R)-Acetoin), l-Arginine iminohydrolase (H2O + | | |
| | | | CAETHG_3021 | L-Arginine <=> NH3 + Citrulline) | | |
| 21 | Yield - | 5 | CAETHG_2210, | dGTP triphosphohydrolase (H2O + dGTP --> H+ + Deoxyguanosine + | 0 | 0.035826 |
| | LMOMA | | CAETHG_2224, | Triphosphate), NADP-dependent electron-bifurcating [FeFe]- | | |
| | | | CAETHG_2475, | hydrogenase (Hyt) (NADP + Oxidizedferredoxin + 2.0H2 <=> | | |
| | | | CAETHG_2798, | NADPH + 3.0H+ + Reducedferredoxin), Alpha acetolactate | | |
| | | | CAETHG_29B2 | decarboxylase (ALCTT --> CO2 + (R)-Acetoin) | | |
| 22 | Yield - | 5 | CAETHG_2210, | NADP-dependent electron-bifurcating [FeFe]-hydrogenase (Hyt) | 0 | 0.035799 |
| | LMOMA | | CAETHG_2224, | (NADP + Oxidizedferredoxin + 2.0H2 <=> NADPH + 3.0H+ + | | |
| | | | CAETHG_2798, | Reducedferredoxin), Alpha-acetolactate decarboxylase (ALCTT --> | | |
| | | | CAETHG_2932, | CO2 + (R)-Acetoin), UMP:pyrophosphate | | |
| | | | CAETHG_3164 | phosphoribosyltransferase (Uracil + PRPP --> PPi + UMP) | | |
| 23 | Yield - | 4 | CAETHG_2210, | NADP-dependent electron-bifurcating [FeFe]-hydrogenase (Hyt) | 0 | 0.035754 |
| | LMOMA | | CAETHG_2224, | (NADP + Oxidizedferredoxin + 2.0H2 <=> NADPH + 3.0H+ + | | |
| | | | CAETHG_2799, | Reducedferredoxin), Alpha-acetolactate decarboxylase (ALCTT --> | | |
| | | | CAETHG_2932 | CO2 + (R)-Acetoin) | | |
| 24 | Yield - | 4 | CAETHG_2210, | NADP-dependent electron-bifurcating [FeFe]-hydrogenase (Hyt) | 0 | 0.035754 |
| | LMOMA | | CAETHG_2224, | (NADP + Oxidizedferredoxin + 2.0H2 <=> NADPH + 3.0H+ + | | |
| | | | CAETHG_2795, | Reducedferredoxin), Alpha-acetolactate decarboxylase (ALCTT --> | | |
| | | | CAETHG_2932 | CO2 + (R)-Acetoin) | | |
| 25 | Yield - | 4 | CAETHG_2210, | NADP-dependent electron-bifurcating [FeFe]-hydrogenase (Hyt) | 0 | 0.035754 |
| | LMOMA | | CAETHG_2224, | (NADP + Oxidizedferredoxin + 2.0H2 <=> NADPH + 3.0H+ + | | |
| | | | CAETHG_2794, | Reducedferredoxin), Alpha-acetolactate decarboxylase (ALCTT --> | | |
| | | | CAETHG_2932 | CO2 + (R)-Acetoin) | | |
| 26 | Yield - | 4 | CAETHG_2210, | NADP-dependent electron-bifurcating [FeFe]-hydrogenase <Hyt) | 0 | 0.035754 |
| | LMOMA | | CAETHG_2224, | (NADP + Oxidizedferredoxin + 2.0H2 <=> NADPH + 3.0H+ + | | |
| | | | CAETHG_2798, | Reducedferredoxin), Alpha-acetolactate decarboxylase (ALCTT --> | | |
| | | | CAETHG_2932 | CO2 + (R)-Acetoin) | | |
| 27 | Yield - | 4 | CAETHG_2210, | NADP-dependent electron-bifurcating [FeFe]-hydrogenase (Hyt) | 0 | 0.035748 |
| | LMOMA | | CAETHG_2224, | (NADP + Oxidizedferredoxin + 2.0H2 <=> NADPH + 3.0H+ + | | |
| | | | CAETHG_2798, | Reducedferredoxin), Alpha-acetolactate decarboxylase (ALCTT --> | | |
| | | | CAETHG_29B2 | CO2 + (R)-Acetoin) | | |
| 28 | Yield - | 5 | CAETHG_2210, | Alpha-acetolactate decarboxylase (ALCTT --> CO2 + (R)-Acetoin), 2- | 0 | 0.034086 |
| | LMOMA | | CAETHG_2211, | Deoxy-D-ribose-5-phosphate acetaldehyde-lyase (deoxyribose-5- | | |
| | | | CAETHG_2224, | phosphate <=> Acetaldehyde + Glyceraldehyde3-phosphate) | | |
| | | | CAETHG_2932, | | | |
| | | | CAETHG_3299 | | | |
| 29 | Yield - | 5 | CAETHG_1757, | Alpha-acetolactate decarboxylase (ALCTT --> CO2 + (R)-Acetoin), 2- | 0 | 0.034086 |
| | LMOMA | | CAETHG_2210, | Deoxy-D-ribose-5-phosphate acetaldehyde-lyase (deoxyribose-5- | | |
| | | | CAETHG_2224, | phosphate <=> Acetaldehyde + Glyceraldehyde3-phosphate) | | |
| | | | CAETHG_2932, | | | |
| | | | CAETHG_3299 | | | |
| 30 | Yield - | 5 | CAETHG_2210, | dCMP aminohydrolase (H2O + H+ + dCMP --> NH3 + dUMP), Alpha- | 0 | 0.034086 |
| | LMOMA | | CAETHG_2224, | acetolactate decarboxylase (ALCTT --> CO2 + (R)-Acetoin), 2-Deoxy- | | |
| | | | CAETHG_2339, | D-ribose-5-phosphate acetaldehyde-lyase (deoxyribose-5- | | |
| | | | CAETHG_2932, | phosphate <=> Acetaldehyde + Glyceraldehyde3-phosphate) | | |
| | | | CAETHG_3299 | | | |
| 31 | Yield - | 5 | CAETHG_2210, | Alpha-acetolactate decarboxylase (ALCTT --> CO2 + (R)-Acetoin), 2- | 0 | 0.034086 |
| | LMOMA | | CAETHG_2224, | Deoxy-D-ribose-5-phosphate acetaldehyde-lyase (deoxyribose-5- | | |
| | | | CAETHG_2932, | phosphate <=> Acetaldehyde + Glyceraldehyde3-phosphate), | | |
| | | | CAETHG_3299, | Arsenate transporter (Arsenate --> Arsenate_ext) | | |
| | | | CAETHG_3985 | | | |
| 32 | Yield - | 5 | CAETHG_2210, | N-Acetylneuraminate pyruvate-lyase (pyruvate-phosphorylating) | 0 | 0.034086 |
| | LMOMA | | CAETHG_2224, | (H2O + Phosphoenolpyruvate + N-Acetyl-D-mannosamine --> | | |
| | | | CAETHG_2633, | Phosphate + H+ + Neu5Ac), Alpha-acetolactate decarboxylase | | |
| | | | CAETHG_2932, | (ALCTT --> CO2 + (R)-Acetoin), 2-Deoxy-D-ribose-5-phosphate | | |
| | | | CAETHG_3299 | acetaldehyde-lyase (deoxyribose-5-phosphate <=> Acetaldehyde + | | |
| | | | | Glyceraldehyde3 phosphate) | | |
| 33 | Yield - | 6 | CAETHG_2210, | Alpha-acetolactate decarboxylase (ALCTT --> CO2 + (R)-Acetoin), 2- | 0 | 0.034086 |
| | LMOMA | | CAETHG_2224, | Deoxy-D-ribose-5-phosphate acetaldehyde-lyase (deoxyribose-5- | | |
| | | | CAETHG_2548, | phosphate <=> Acetaldehyde + Glyceraldehyde3-phosphate), | | |
| | | | CAETHG_2932, | thiazole phosphate synthesis (ATP + L-Tyrosine + L-Cysteine + 1- | | |
| | | | CAETHG_3299, | deoxy-D-xylulose5-phosphate --> H2O + CO2 + PPi + AMP + L- | | |
| | | | CAETHG_0832 | Alanine + 4-Methyl-5--2-phosphoethyl-thiazole + 4-Hydroxy- | | |
| | | | | benzylalcohol) | | |
| 34 | Yield - | 7 | CAETHG_2210, | Alpha-acetolactate decarboxylase (ALCTT --> CO2 + (R)-Acetoin), 2- | 0 | 0.034086 |
| | LMOMA | | CAETHG_2224, | Deoxy-D-ribose-5-phosphate acetaldehyde-lyase (deoxyribose-5- | | |
| | | | CAETHG_2932, | phosphate <=> Acetaldehyde + Glyceraldehyde3-phosphate) | | |
| | | | CAETHG_3299, | | | |
| | | | CAETHG_3850, | | | |
| | | | CAETHG_0417, | | | |
| | | | CAETHG_0461 | | | |
| 35 | Yield - | 4 | CAETHG_2210, | Alpha-acetolactate decarboxylase (ALCTT --> CO2 + (R)-Acetoin), 2- | 0 | 0.034086 |
| | LMOMA | | CAETHG_2224, | Deoxy-D-ribose-5-phosphate acetaldehyde-lyase (deoxyribose-5- | | |
| | | | CAETHG_2932, | phosphate <=> Acetaldehyde + Glyceraldehyde3-phosphate) | | |
| | | | CAETHG_3299 | | | |
| 36 | Yield - | 5 | CAETHG_2210, | Alpha-acetolactate decarboxylase (ALCTT --> CO2 + (R)-Acetoin), 2- | 0 | 0.034086 |
| | LMOMA | | CAETHG_2224, | Deoxy-D-ribose-5-phosphate acetaldehyde-lyase (deoxyribose-5- | | |
| | | | CAETHG_2618, | phosphate <=> Acetaldehyde + Glyceraldehyde3-phosphate) | | |
| | | | CAETHG_2932, | | | |
| | | | CAETHG_3299 | | | |
| 37 | Yield - | 3 | CAETHG_2107, | Potassium uptake (K+_ext <=> K+), Alpha-acetolactate | 0 | 0.026043 |
| | LMOMA | | CAETHG_2932, | decarboxylase (ALCTT --> CO2 + (R)-Acetoin), L-Arginine | | |
| | | | CAETHG_3021 | iminohydrolase (H2O + L-Arginine <=> NH3 + Citrulline) | | |
| 38 | Yield - | 2 | CAETHG_2932, | Alpha-acetolactate decarboxylase (ALCTT --> CO2 + (R)-Acetoin), L- | 0 | 0.025995 |
| | LMOMA | | CAETHG_3021 | Arginine iminohydrolase (H2O + L-Arginine <=> NH3 + Citrulline) | | |
| 39 | Yield - | 2 | CAETHG_2107, | Potassium uptake (K+_ext <=> K+), Alpha-acetolactate | 0 | 0.024423 |
| | LMOMA | | CAETHG_2932 | decarboxylase (ALCTT --> CO2 + (R)-Acetoin) | | |
| 40 | Yield - | 1 | CAETHG_2932 | Alpha-acetolactate decarboxylase (ALCTT --> CO2 + (R)-Acetoin) | 0 | 0.024375 |
| | LMOMA | | | | | |
| 41 | Yield - | 2 | CAETHG_1225, | L-serine ammonia-lyase (L-Serine --> NH3 + Pyruvate), N- | 0 | 0.021384 |
| | LMOMA | | CAETHG_0160 | Ribosylnicotinamide:orthophosphate ribosyltransferase | | |
| | | | | (Phosphate + N-Ribosylnicotinamide <=> Nicotinamide + Ribose 1- | | |
| | | | | phosphate) | | |
| 42 | Yield - | 1 | CAETHG_0160 | N-Ribosylnicotinamide:orthophosphate ribosyltransferase | 0 | 0.021204 |
| | LMOMA | | | (Phosphate + N-Ribosylnicotinamide <=> Nicotinamide + Ribose 1- | | |
| | | | | phosphate) | | |
| 43 | Yield - | 1 | CAETHG_1371 | Adenosine 5′-monophosphate phosphohydrolase (H2O + AMP <=> | 0 | 0.021204 |
| | LMOMA | | | Phosphate + H+ + Adenosine) | | |
| 44 | Yield - | 1 | CAETHG_1270 | AMP:pyrophosphate phosphoribosyltransferase (PPi + AMP <=> | 0 | 0.021204 |
| | LMOMA | | | PRPP + Adenine) | | |
| 45 | Yield - | 1 | CAETHG_3924 | D-Ribose 1,5-phosphomutase (Ribose 1-phosphate <=> ribose-5- | 0 | 0.021204 |
| | LMOMA | | | phosphate) | | |
| 46 | Yield - | 2 | CAETHG_2721, | Phosphoenolpyruvate carboxykinase (PPCK) (ATP + Oxaloacetate + | 0 | 0.021012 |
| | LMOMA | | CAETHG_3021 | H+ --> ADP + CO2 + Phosphoenolpyruvate), L-Arginine | | |
| | | | | iminohydrolase (H2O + L-Arginine <=> NH3 + Citrulline) | | |
| 47 | Yield - | 2 | CAETHG_2107, | Potassium uptake (K+_ext <=> K+), Phosphoenolpyruvate | 0 | 0.020931 |
| | LMOMA | | CAETHG_2721 | carboxykinase (PPCK) (ATP + Oxaloacetate + H+ --> ADP + CO2 + | | |
| | | | | Phosphoenolpyruvate) | | |
| 48 | Yield - | 1 | CAETHG_2721 | Phosphoenolpyruvate carboxykinase (PPCK) (ATP + Oxaloacetate + | 0 | 0.020928 |
| | LMOMA | | | H+ --> ADP + CO2 + Phosphoenolpyruvate) | | |
| 49 | Yield - | 2 | CAETHG_2475, | dGTP triphosphohydrolase (H2O + dGTP --> H+ + Deoxyguanosine + | 0 | 0.020073 |
| | LMOMA | | CAETHG_3021 | Triphosphate), L-Arginine iminohydrolase (H2O + L-Arginine <=> | | |
| | | | | NH3 + Citrulline) | | |
| 50 | Yield - | 1 | CAETHG_3021 | L-Arginine iminohydrolase (H2O + L-Arginine <=> NH3 + Citrulline) | 0 | 0.020058 |
| | LMOMA | | | | | |

### Example 7

This example describes disruptions for improved production of 1,3-butanediol in Wood-Ljungdahl microorganisms. Metabolic modeling experiments were performed as described in Example 1. Production of 1,3-butanediol in Wood-Ljungdahl microorganisms is described, e.g., in WO 2017/0066498. The following pathway was used to model 1,3-butanediol production herein: 2.0 Acetyl-CoA-->CoA+Acetoacetyl-CoA; NADPH+H++Acetoacetyl-CoA-->NADP+(R)-3-Hydroxybutyryl-CoA; Phosphate+(R)-3-Hydroxybutyryl-CoA-->CoA+(R)-3-hydroxybutyryl-Phosphate; ADP+(R)-3-hydroxybutyryl-Phosphate-->ATP+(R)-3-Hydroxybutyrate; (R)-3-Hydroxybutyrate+Reducedferredoxin-->Oxidizedferredoxin+(R)-3-hydroxybutyraldehyde; NADPH+H++(R)-3-hydroxybutyraldehyde-->NADP+13BDO; NADH+H++(R)-3-hydroxybutyraldehyde-->NAD+13BDO; 13BDO-->13BDO_ext.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | BPCY- | 4 | CAETHG_2751, | Citramalate synthase (H2O + Pyruvate + Acetyl-CoA <=> CoA + | 0.05315 | 0.005908 |
| | FBA | | CAETHG_2909, | Citramalate), ATP: pyruvate, orthophosphate phosphotransferase | | |
| | | | CAETHG_3293, | (ATP + Phosphate + Pyruvate + H+ --> PPi + AMP + | | |
| | | | CAETHG_3358 | Phosphoenolpyruvate), Cysteine desulfhydrase (H2O + L- | | |
| | | | | Cysteine <=> NH3 + Pyruvate + H2S), Phosphate transacetylase | | |
| | | | | (Phosphate + Acetyl-CoA + H+ <=> CoA + Acetylphosphate) | | |
| 2 | BPCY- | 3 | CAETHG_2909, | ATP: pyruvate, orthophosphate phosphotransferase (ATP + | 0.05193 | 0.005808 |
| | FBA | | CAETHG_3293, | Phosphate + Pyruvate + H+ --> PPi + AMP + | | |
| | | | CAETHG_3358 | Phosphoenolpyruvate), Cysteine desulfhydrase (H2O + L- | | |
| | | | | Cysteine <=> NH3 + Pyruvate + H2S), Phosphate transacetylase | | |
| | | | | (Phosphate + Acetyl-CoA + H+ <=> CoA + Acetylphosphate) | | |
| 3 | BPCY- | 3 | CAETHG_2751, | Citramalate synthase (H2O + Pyruvate + Acetyl-CoA <=> CoA + | 0.05085 | 0.005774 |
| | FBA | | CAETHG_2909, | Citramalate), ATP: pyruvate, orthophosphate phosphotransferase | | |
| | | | CAETHG_3358 | (ATP + Phosphate + Pyruvate + H+ --> PPi + AMP + | | |
| | | | | Phosphoenolpyruvate), Phosphate transacetylase (Phosphate + | | |
| | | | | Acetyl-CoA + H+ <=> CoA + Acetylphosphate) | | |
| 4 | BPCY- | 3 | CAETHG_2751, | Citramalate synthase (H2O + Pyruvate + Acetyl-CoA <=> CoA + | 0.05085 | 0.005774 |
| | FBA | | CAETHG_2909, | Citramalate), ATP: pyruvate, orthophosphate phosphotransferase | | |
| | | | CAETHG_3359 | (ATP + Phosphate + Pyruvate + H+ --> PPi + AMP + | | |
| | | | | Phosphoenolpyruvate), ATP: acetate phosphotransferase (ATP + | | |
| | | | | H+ + Propionate --> ADP + Propionyl phosphate) | | |
| 5 | BPCY- | 3 | CAETHG_2909, | ATP: pyruvate, orthophosphate phosphotransferase (ATP + | 0.05054 | 0.005734 |
| | FBA | | CAETHG_3358, | Phosphate + Pyruvate + H+ --> PPi + AMP + | | |
| | | | CAETHG_0498 | Phosphoenolpyruvate), Phosphate transacetylase (Phosphate + | | |
| | | | | Acetyl-CoA + H+ <=> CoA + Acetylphosphate), Cystathionine | | |
| | | | | beta lyase (H2O + Cystathionine --> NH3 + Pyruvate + | | |
| | | | | Homocysteine) | | |
| 6 | BPCY- | 2 | CAETHG_2909, | ATP: pyruvate, orthophosphate phosphotransferase (ATP + | 0.04960 | 0.005672 |
| | FBA | | CAETHG_3359 | Phosphate + Pyruvate + H+ --> PPi + AMP + | | |
| | | | | Phosphoenolpyruvate), ATP: acetate phosphotransferase (ATP + | | |
| | | | | H+ + Propionate --> ADP + Propionyl phosphate) | | |
| 7 | BPCY- | 2 | CAETHG_2909, | ATP: pyruvate, orthophosphate phosphotransferase (ATP + | 0.04960 | 0.005672 |
| | FBA | | CAETHG_3358 | Phosphate + Pyruvate + H+ --> PPi + AMP + | | |
| | | | | Phosphoenolpyruvate), Phosphate transacetylase (Phosphate + | | |
| | | | | Acetyl-CoA + H+ <=> CoA + Acetylphosphate) | | |
| 8 | BPCY- | 3 | CAETHG_2751, | Citramalate synthase (H2O + Pyruvate + Acetyl-CoA <=> CoA + | 0.04193 | 0.004892 |
| | FBA | | CAETHG_3293, | Citramalate), Cysteine desulfhydrase (H2O + L-Cysteine <=> | | |
| | | | CAETHG_3358 | NH3 + Pyruvate + H2S), Phosphate transacetylase (Phosphate + | | |
| | | | | Acetyl-CoA + H+ <=> CoA + Acetylphosphate) | | |
| 9 | BPCY- | 2 | CAETHG_3293, | Cysteine desulfhydrase (H2O + L-Cysteine <=> NH3 + Pyruvate + | 0.04068 | 0.004788 |
| | FBA | | CAETHG_3359 | H2S), ATP: acetate phosphotransferase (ATP + H+ + | | |
| | | | | Propionate --> ADP + Propionyl phosphate) | | |
| 10 | BPCY- | 2 | CAETHG_3293, | Cysteine desulfhydrase (H2O + L-Cysteine <=> NH3 + Pyruvate + | 0.04068 | 0.004788 |
| | FBA | | CAETHG_3358 | H2S), Phosphate transacetylase (Phosphate + Acetyl-CoA + | | |
| | | | | H+ <=> CoA + Acetylphosphate) | | |
| 11 | BPCY- | 2 | CAETHG_2751, | Citramalate synthase (H2O + Pyruvate + Acetyl-CoA <=> CoA + | 0.03923 | 0.004684 |
| | FBA | | CAETHG_3359 | Citramalate), ATP: acetate phosphotransferase (ATP + H+ + | | |
| | | | | Propionate --> ADP + Propionyl phosphate) | | |
| 12 | BPCY- | 2 | CAETHG_2751, | Citramalate synthase (H2O + Pyruvate + Acetyl-CoA <=> CoA + | 0.03923 | 0.004684 |
| | FBA | | CAETHG_3358 | Citramalate), Phosphate transacetylase (Phosphate + | | |
| | | | | Acetyl-CoA + H+ <=> CoA + Acetylphosphate) | | |
| 13 | BPCY- | 2 | CAETHG_3359, | ATP: acetate phosphotransferase (ATP + H+ + Propionate --> | 0.03904 | 0.004667 |
| | FBA | | CAETHG_0498 | ADP + Propionyl phosphate), Cystathionine beta lyase (H2O + | | |
| | | | | Cystathionine --> NH3 + Pyruvate + Homocysteine) | | |
| 14 | BPCY- | 2 | CAETHG_3358, | Phosphate transacetylase (Phosphate + Acetyl-CoA + H+ <=> | 0.03904 | 0.004667 |
| | FBA | | CAETHG_0498 | CoA + Acetylphosphate), Cystathionine beta lyase (H2O + | | |
| | | | | Cystathionine --> NH3 + Pyruvate + Homocysteine) | | |
| 15 | BPCY- | 1 | CAETHG_3359 | ATP: acetate phosphotransferase (ATP + H+ + Propionate --> | 0.03795 | 0.004575 |
| | FBA | | | ADP + Propionyl phosphate) | | |
| 16 | BPCY- | 1 | CAETHG_3358 | Phosphate transacetylase (Phosphate + Acetyl-CoA + H+ <=> | 0.03795 | 0.004575 |
| | FBA | | | CoA + Acetylphosphate) | | |

### Example 8

This example describes disruptions for improved production of 2,3-butanediol in Wood-Ljungdahl microorganisms. Metabolic modeling experiments were performed as described in Example 1. 2,3-butanediol is a native product of at least some Wood-Ljungdahl microorganisms.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | Yield- | 5 | CAETHG_1147, | Lactate dehydrogenase (NAD + D-Lactate <=> NADH + | 0.033681 | 0.049774 |
| | LMOMA | | CAETHG_2753, | Pyruvate + H+), Isocitrate dehydrogenase (NAD + Isocitrate <=> | | |
| | | | CAETHG_3359, | NADH + CO2 + 2-Oxoglutarate + H+), ATP: acetate | | |
| | | | CAETHG_3510, | phosphotransferase (ATP + H+ + Propionate --> ADP + | | |
| | | | CAETHG_0909 | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Prephenate: NAD+ | | |
| | | | | oxidoreductase(decarboxylating) (NAD + Prephenate --> | | |
| | | | | NADH + CO2 + p-hydroxyphenylpyruvate) | | |
| 2 | Yield- | 4 | CAETHG_1147, | Lactate dehydrogenase (NAD + D-Lactate <=> NADH + | 0.033681 | 0.049768 |
| | LMOMA | | CAETHG_2753, | Pyruvate + H+), Isocitrate dehydrogenase (NAD + Isocitrate <=> | | |
| | | | CAETHG_3359, | NADH + CO2 + 2-Oxoglutarate + H+), ATP: acetate | | |
| | | | CAETHG_3510 | phosphotransferase (ATP + H+ + Propionate --> ADP + | | |
| | | | | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 3 | Yield- | 4 | CAETHG_0248, | L-lactate reversible transport via proton symport (H+_ext + | 0.033681 | 0.049768 |
| | LMOMA | | CAETHG_2753, | L-Lactate_ext <=> H+ + L-Lactate), Isocitrate dehydrogenase | | |
| | | | CAETHG_3359, | (NAD + Isocitrate <=> NADH + CO2 + 2-Oxoglutarate + H+), | | |
| | | | CAETHG_3510 | ATP: acetate phosphotransferase (ATP + H+ + Propionate --> | | |
| | | | | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 4 | Yield- | 3 | CAETHG_0248, | L-lactate reversible transport via proton symport (H+_ext + | 0.03068 | 0.04884 |
| | LMOMA | | CAETHG_3359, | L-Lactate_ext <=> H+ + L-Lactate), ATP: acetate | | |
| | | | CAETHG_3510 | phosphotransferase (ATP + H+ + Propionate --> ADP + | | |
| | | | | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 5 | Yield- | 4 | CAETHG_0248, | L-lactate reversible transport via proton symport (H+_ext + | 0 | 0.026026 |
| | LMOMA | | CAETHG_2721, | L-Lactate_ext <=> H+ + L-Lactate), Phosphoenolpyruvate | | |
| | | | CAETHG_3327, | carboxykinase (PPCK) (ATP + Oxaloacetate + H+ --> ADP + | | |
| | | | CAETHG_3359 | CO2 + Phosphoenolpyruvate), phosphate ABC transporter | | |
| | | | | permease protein (PPi + H+ --> PPi_ext + H+_ext), | | |
| | | | | ATP: acetate phosphotransferase (ATP + H+ + Propionate --> | | |
| | | | | ADP + Propionyl phosphate) | | |
| 6 | Yield- | 4 | CAETHG_0248, | L-lactate reversible transport via proton symport (H+_ext + | 0 | 0.02553 |
| | LMOMA | | CAETHG_3359, | L-Lactate_ext <=> H+ + L-Lactate), ATP: acetate | | |
| | | | CAETHG_3924, | phosphotransferase (ATP + H+ + Propionate --> ADP + | | |
| | | | CAETHG_0476 | Propionyl phosphate), D-Ribose 1,5-phosphomutase (Ribose | | |
| | | | | 1-phosphate <=> ribose-5-phosphate), S- | | |
| | | | | aminomethyldihydrolipoylprotein: (66)-tetrahydrofolate | | |
| | | | | (Tetrahydrofolate + S-Aminomethyldihydrolipoylprotein <=> | | |
| | | | | NH3 + 5-10-Methylenetetrahydrofolate + | | |
| | | | | Dihydrolipolprotein) | | |
| 7 | Yield- | 4 | CAETHG_0248, | L-lactate reversible transport via proton symport (H+_ext + | 0 | 0.02553 |
| | LMOMA | | CAETHG_3359, | L-Lactate_ext <=> H+ + L-Lactate), ATP: acetate | | |
| | | | CAETHG_3924, | phosphotransferase (ATP + H+ + Propionate --> ADP + | | |
| | | | CAETHG_0475 | Propionyl phosphate), D-Ribose 1,5-phosphomutase (Ribose | | |
| | | | | 1-phosphate <=> ribose-5-phosphate), S- | | |
| | | | | aminomethyldihydrolipoylprotein: (6S)-tetrahydrofolate | | |
| | | | | (Tetrahydrofolate + S-Aminomethyldihydrolipoylprotein <=> | | |
| | | | | NH3 + 5-10-Methylenetetrahydrofolate + | | |
| | | | | Dihydrolipolprotein) | | |
| 8 | Yield- | 4 | CAETHG_0248, | L-lactate reversible transport via proton symport (H+_ext + | 0 | 0.02553 |
| | LMOMA | | CAETHG_3359, | L-Lactate_ext <=> H+ + L-Lactate), ATP: acetate | | |
| | | | CAETHG_3924, | phosphotransferase (ATP + H+ + Propionate --> ADP + | | |
| | | | CAETHG_0474 | Propionyl phosphate), D-Ribose 1,5-phosphomutase (Ribose | | |
| | | | | 1-phosphate <=> ribose-5-phosphate), Glycinedipoylprotein | | |
| | | | | oxidoreductase (decarboxylating and acceptor- | | |
| | | | | aminomethylating) (Glycine + H+ + Lipoylprotein --> CO2 + S- | | |
| | | | | Aminomethyldihydrolipoylprotein) | | |
| 9 | Yield- | 4 | CAETHG_1270, | AMP: pyrophosphate phosphoribosyltransferase (PPi + AMP <=> | 0 | 0.025452 |
| | LMOMA | | CAETHG_0248, | PRPP + Adenine), L-lactate reversible transport via | | |
| | | | CAETHG_2753, | proton symport (H+_ext + L-Lactate_ext <=> H+ + L-Lactate), | | |
| | | | CAETHG_3359 | Isocitrate dehydrogenase (NAD + Isocitrate <=> NADH + | | |
| | | | | CO2 + 2-Oxoglutarate + H+), ATP: acetate phosphotransferase | | |
| | | | | (ATP + H+ + Propionate --> ADP + Propionyl phosphate) | | |
| 10 | Yield- | 4 | CAETHG_0248, | L-lactate reversible transport via proton symport (H+_ext + | 0 | 0.025452 |
| | LMOMA | | CAETHG_2753, | L-Lactate_ext <=> H+ + L-Lactate), Isocitrate dehydrogenase | | |
| | | | CAETHG_3359, | (NAD + Isocitrate <=> NADH + CO2 + 2-Oxoglutarate + H+), | | |
| | | | CAETHG_3924 | ATP: acetate phosphotransferase (ATP + H+ + Propionate --> | | |
| | | | | ADP + Propionyl phosphate), D-Ribose 1,5-phosphomutase | | |
| | | | | (Ribose 1-phosphate <=> ribose-5-phosphate) | | |
| 11 | Yield- | 4 | CAETHG_0248, | L-lactate reversible transport via proton symport (H+_ext + | 0 | 0.025422 |
| | LMOMA | | CAETHG_3359, | L-Lactate_ext <=> H+ + L-Lactate), ATP: acetate | | |
| | | | CAETHG_3924, | phosphotransferase (ATP + H+ + Propionate --> ADP + | | |
| | | | CAETHG_0498 | Propionyl phosphate), D-Ribose 1,5-phosphomutase (Ribose | | |
| | | | | 1-phosphate <=> ribose-5-phosphate), Cystathionine beta | | |
| | | | | synthase (L-Serine + Homocysteine <=> H2O + Cystathionine) | | |
| 12 | Yield- | 3 | CAETHG_1371, | 5′-nucleotidase (dUMP) (H2O + dUMP --> Phosphate + H+ + | 0 | 0.025412 |
| | LMOMA | | CAETHG_0248, | Deoxyuridine), L-lactate reversible transport via proton | | |
| | | | CAETHG_3359 | symport (H+_ext + L-Lactate_ext <=> H+ + L-Lactate), | | |
| | | | | ATP: acetate phosphotransferase (ATP + H+ + Propionate --> | | |
| | | | | ADP + Propionyl phosphate) | | |
| 13 | Yield- | 3 | CAETHG_1147, | Lactate dehydrogenase (NAD + D-Lactate <=> NADH + | 0 | 0.025412 |
| | LMOMA | | CAETHG_3359, | Pyruvate + H+), ATP: acetate phosphotransferase (ATP + H+ + | | |
| | | | CAETHG_3924 | Propionate --> ADP + Propionyl phosphate), D-Ribose 1,5- | | |
| | | | | phosphomutase (Ribose 1-phosphate <=> ribose-5- | | |
| | | | | phosphate) | | |
| 14 | Yield- | 3 | CAETHG_0160, | L-lactate reversible transport via proton symport (H+_ext + | 0 | 0.025412 |
| | LMOMA | | CAETHG_0248, | L-Lactate_ext <=> H+ + L-Lactate), ATP: acetate | | |
| | | | CAETHG_3359 | phosphotransferase (ATP + H+ + Propionate --> ADP + | | |
| | | | | Propionyl phosphate) | | |
| 15 | Yield- | 3 | CAETHG_0248, | L-lactate reversible transport via proton symport (H+_ext + | 0 | 0.025412 |
| | LMOMA | | CAETHG_3359, | L-Lactate_ext <=> H+ + L-Lactate), ATP: acetate | | |
| | | | CAETHG_3924 | phosphotransferase (ATP + H+ + Propionate --> ADP + | | |
| | | | | Propionyl phosphate), D-Ribose 1,5-phosphomutase (Ribose | | |
| | | | | 1-phosphate <=> ribose-5-phosphate) | | |
| 16 | Yield- | 3 | CAETHG_0248, | L-lactate reversible transport via proton symport (H+_ext + | 0 | 0.02517 |
| | LMOMA | | CAETHG_2721, | L-Lactate_ext <=> H+ + L-Lactate), Phosphoenolpyruvate | | |
| | | | CAETHG_3359 | carboxykinase (PPCK) (ATP + Oxaloacetate + H+ --> ADP + | | |
| | | | | CO2 + Phosphoenolpyruvate), ATP: acetate | | |
| | | | | phosphotransferase (ATP + H+ + Propionate --> ADP + | | |
| | | | | Propionyl phosphate) | | |
| 17 | Yield- | 3 | CAETHG_1147, | Lactate dehydrogenase (NAD + D-Lactate <=> NADH + | 0 | 0.024914 |
| | LMOMA | | CAETHG_2753, | Pyruvate + H+), Isocitrate dehydrogenase (NAD + Isocitrate <=> | | |
| | | | CAETHG_3359 | NADH + CO2 + 2-Oxoglutarate + H+), ATP: acetate | | |
| | | | | phosphotransferase (ATP + H+ + Propionate --> ADP + | | |
| | | | | Propionyl phosphate) | | |
| 18 | Yield- | 3 | CAETHG_0248, | L-lactate reversible transport via proton symport (H+_ext + | 0 | 0.024914 |
| | LMOMA | | CAETHG_2753, | L-Lactate_ext <=> H+ + L-Lactate), Isocitrate dehydrogenase | | |
| | | | CAETHG_3359 | (NAD + Isocitrate <=> NADH + CO2 + 2-Oxoglutarate + H+), | | |
| | | | | ATP: acetate phosphotransferase (ATP + H+ + Propionate --> | | |
| | | | | ADP + Propionyl phosphate) | | |
| 19 | Yield- | 3 | CAETHG_0248, | L-lactate reversible transport via proton symport (H+_ext + | 0 | 0.0249 |
| | LMOMA | | CAETHG_3299, | L-Lactate_ext <=> H+ + L-Lactate), 2-Deoxy-D-ribose-5- | | |
| | | | CAETHG_3359 | phosphate acetaldehyde-lyase (deoxyribose-5-phosphate <=> | | |
| | | | | Acetaldehyde + Glyceraldehyde3-phosphate), | | |
| | | | | ATP: acetate phosphotransferase (ATP + H+ + Propionate --> | | |
| | | | | ADP + Propionyl phosphate) | | |
| 20 | Yield- | 4 | CAETHG_0248, | L-lactate reversible transport via proton symport (H+_ext + | 0 | 0.0249 |
| | LMOMA | | CAETHG_2475, | L-Lactate_ext <=> H+ + L-Lactate), dGTP triphosphohydrolase | | |
| | | | CAETHG_3164, | (H2O + dGTP --> H+ + Deoxyguanosine + Triphosphate), | | |
| | | | CAETHG_3359 | UMP: pyrophosphate phosphoribosyltransferase (Uracil + | | |
| | | | | PRPP --> PPi + UMP), ATP: acetate phosphotransferase (ATP + | | |
| | | | | H+ + Propionate --> ADP + Propionyl phosphate) | | |
| 21 | Yield- | 3 | CAETHG_0248, | L-lactate reversible transport via proton symport (H+_ext + | 0 | 0.024882 |
| | LMOMA | | CAETHG_2475, | L-Lactate_ext <=> H+ + L-Lactate), dGTP triphosphohydrolase | | |
| | | | CAETHG_3359 | (H2O + dGTP --> H+ + Deoxyguanosine + Triphosphate), | | |
| | | | | ATP: acetate phosphotransferase (ATP + H+ + Propionate --> | | |
| | | | | ADP + Propionyl phosphate) | | |
| 22 | Yield- | 3 | CAETHG_1607, | S-aminomethyldihydrolipoylprotein: (6S)-tetrahydrofolate | 0 | 0.024808 |
| | LMOMA | | CAETHG_0248, | (Tetrahydrofolate + S-Aminomethyldihydrolipoylprotein <=> | | |
| | | | CAETHG_3359 | NH3 + 5-10-Methylenetetrahydrofolate + | | |
| | | | | Dihydrolipolprotein), L-lactate reversible transport via | | |
| | | | | proton symport (H+_ext + L-Lactate_ext <=> H+ + L-Lactate), | | |
| | | | | ATP: acetate phosphotransferase (ATP + H+ + Propionate --> | | |
| | | | | ADP + Propionyl phosphate) | | |
| 23 | Yield- | 3 | CAETHG_0248, | L-lactate reversible transport via proton symport (H+_ext + | 0 | 0.024808 |
| | LMOMA | | CAETHG_3359, | L-Lactate_ext <=> H+ + L-Lactate), ATP: acetate | | |
| | | | CAETHG_0475 | phosphotransferase (ATP + H+ + Propionate --> ADP + | | |
| | | | | Propionyl phosphate), S- | | |
| | | | | aminomethyldihydrolipoylprotein: (6S)-tetrahydrofolate | | |
| | | | | (Tetrahydrofolate + S-Aminomethyldihydrolipoylprotein <=> | | |
| | | | | NH3 + 5-10-Methylenetetrahydrofolate + | | |
| | | | | Dihydrolipolprotein) | | |
| 24 | Yield- | 3 | CAETHG_0248, | L-lactate reversible transport via proton symport (H+_ext + | 0 | 0.024808 |
| | LMOMA | | CAETHG_3359, | L-Lactate_ext <=> H+ + L-Lactate), ATP: acetate | | |
| | | | CAETHG_0474 | phosphotransferase (ATP + H+ + Propionate --> ADP + | | |
| | | | | Propionyl phosphate), Glycinedipoylprotein oxidoreductase | | |
| | | | | (decarboxylating and acceptor-aminomethylating) (Glycine + | | |
| | | | | H+ + Lipoylprotein --> CO2 + 5- | | |
| | | | | Aminomethyldihydrolipoylprotein) | | |
| 25 | Yield- | 3 | CAETHG_0248, | L-lactate reversible transport via proton symport (H+_ext + | 0 | 0.024802 |
| | LMOMA | | CAETHG_3327, | L-Lactate_ext <=> H+ + L-Lactate), phosphate ABC | | |
| | | | CAETHG_3359 | transporter permease protein (PPi + H+ --> PPi_ext + | | |
| | | | | H+_ext), ATP: acetate phosphotransferase (ATP + H+ + | | |
| | | | | Propionate --> ADP + Propionyl phosphate) | | |
| 26 | Yield- | 3 | CAETHG_0248, | L-lactate reversible transport via proton symport (H+_ext + | 0 | 0.024788 |
| | LMOMA | | CAETHG_3359, | L-Lactate_ext <=> H+ + L-Lactate), ATP: acetate | | |
| | | | CAETHG_0473 | phosphotransferase (ATP + H+ + Propionate --> ADP + | | |
| | | | | Propionyl phosphate), Glycinedipoylprotein oxidoreductase | | |
| | | | | (decarboxylating and acceptor-aminomethylating) (Glycine + | | |
| | | | | H+ + Lipoylprotein --> CO2 + 5- | | |
| | | | | Aminomethyldihydrolipoylprotein) | | |
| 27 | Yield- | 3 | CAETHG_1147, | Lactate dehydrogenase (NAD + D-Lactate <=> NADH + | 0 | 0.024756 |
| | LMOMA | | CAETHG_3359, | Pyruvate + H+), ATP: acetate phosphotransferase (ATP + H+ + | | |
| | | | CAETHG_0498 | Propionate --> ADP + Propionyl phosphate), Cystathionine | | |
| | | | | beta synthase (L-Serine + Homocysteine <=> H2O + | | |
| | | | | Cystathionine) | | |
| 28 | Yield- | 3 | CAETHG_0248, | L-lactate reversible transport via proton symport (H+_ext + | 0 | 0.024426 |
| | LMOMA | | CAETHG_3164, | L-Lactate_ext <=> H+ + L-Lactate), UMP: pyrophosphate | | |
| | | | CAETHG_3359 | phosphoribosyltransferase (Uracil + PRPP --> PPi + UMP), | | |
| | | | | ATP: acetate phosphotransferase (ATP + H+ + Propionate --> | | |
| | | | | ADP + Propionyl phosphate) | | |
| 29 | Yield- | 2 | CAETHG_1147, | Lactate dehydrogenase (NAD + D-Lactate <=> NADH + | 0 | 0.024408 |
| | LMOMA | | CAETHG_3359 | Pyruvate + H+), ATP: acetate phosphotransferase (ATP + H+ + | | |
| | | | | Propionate --> ADP + Propionyl phosphate) | | |
| 30 | Yield- | 2 | CAETHG_0248, | L-lactate reversible transport via proton symport (H+_ext + | 0 | 0.024408 |
| | LMOMA | | CAETHG_3359 | L-Lactate_ext <=> H+ + L-Lactate), ATP: acetate | | |
| | | | | phosphotransferase (ATP + H+ + Propionate --> ADP + | | |
| | | | | Propionyl phosphate) | | |
| 31 | Yield- | 4 | CAETHG_0160, | L-lactate reversible transport via proton symport (H+_ext + | 0 | 0.013792 |
| | LMOMA | | CAETHG_0248, | L-Lactate_ext <=> H+ + L-Lactate), Isocitrate dehydrogenase | | |
| | | | CAETHG_2753, | (NAD + Isocitrate <=> NADH + CO2 + 2-Oxoglutarate + H+), | | |
| | | | CAETHG_3327 | phosphate ABC transporter permease protein (PPi + H+ --> | | |
| | | | | PPi_ext + H+_ext) | | |
| 32 | Yield- | 4 | CAETHG_0248, | L-lactate reversible transport via proton symport (H+_ext + | 0 | 0.013792 |
| | LMOMA | | CAETHG_2753, | L-Lactate_ext <=> H+ + L-Lactate), Isocitrate dehydrogenase | | |
| | | | CAETHG_3327, | (NAD + Isocitrate <=> NADH + CO2 + 2-Oxoglutarate + H+), | | |
| | | | CAETHG_3924 | phosphate ABC transporter permease protein (PPi + H+ --> | | |
| | | | | PPi_ext + H+_ext), D-Ribose 1,5-phosphomutase (Ribose 1- | | |
| | | | | phosphate <=> ribose-5-phosphate) | | |
| 33 | Yield- | 4 | CAETHG_1270, | AMP: pyrophosphate phosphoribosyltransferase (PPi + AMP <=> | 0 | 0.013792 |
| | LMOMA | | CAETHG_0248, | PRPP + Adenine), L-lactate reversible transport via | | |
| | | | CAETHG_2753, | proton symport (H+_ext +L-Lactate_ext <=> H+ + L-Lactate), | | |
| | | | CAETHG_3327 | Isocitrate dehydrogenase (NAD + Isocitrate <=> NADH + | | |
| | | | | CO2 + 2-Oxoglutarate + H+), phosphate ABC transporter | | |
| | | | | permease protein (PPi + H+ --> PPi_ext + H+_ext) | | |
| 34 | Yield- | 4 | CAETHG_2753, | Isocitrate dehydrogenase (NAD + Isocitrate <=> NADH + | 0 | 0.013708 |
| | LMOMA | | CAETHG_3359, | CO2 + 2-Oxoglutarate + H+), ATP: acetate phosphotransferase | | |
| | | | | (ATP + H+ + Propionate --> ADP + Propionyl phosphate), D- | | |
| | | | CAETHG_3924, | Ribose 1,5-phosphomutase (Ribose 1-phosphate <=> ribose- | | |
| | | | CAETHG_0498 | 5-phosphate), Cystathionine beta synthase (L-Serine + | | |
| | | | | Homocysteine <=> H2O + Cystathionine) | | |
| 35 | Yield- | 3 | CAETHG_2753, | Isocitrate dehydrogenase (NAD + Isocitrate <=> NADH + | 0 | 0.0137 |
| | LMOMA | | CAETHG_3359, | CO2 + 2-Oxoglutarate + H+), ATP: acetate phosphotransferase | | |
| | | | CAETHG_3924 | (ATP + H+ + Propionate --> ADP + Propionyl phosphate), D- | | |
| | | | | Ribose 1,5-phosphomutase (Ribose 1-phosphate <=> ribose- | | |
| | | | | 5-phosphate) | | |
| 36 | Yield- | 3 | CAETHG_1270, | AMP: pyrophosphate phosphoribosyltransferase (PPi + | 0 | 0.0137 |
| | LMOMA | | CAETHG_2753, | AMP <=> PRPP + Adenine), Isocitrate dehydrogenase (NAD + | | |
| | | | CAETHG_3359 | Isocitrate <=> NADH + CO2 + 2-Oxoglutarate + H+), | | |
| | | | | ATP: acetate phosphotransferase (ATP + H+ + Propionate --> | | |
| | | | | ADP + Propionyl phosphate) | | |
| 37 | Yield- | 3 | 0, | Acetyl-CoA: carbon-dioxide ligase (ADP-forming) (ATP + | 0 | 0.013628 |
| | LMOMA | | CAETHG_2753, | Acetyl-CoA + H2CO3 --> ADP + Phosphate + H+ + Malonyl- | | |
| | | | CAETHG_3359 | CoA), Isocitrate dehydrogenase (NAD + Isocitrate <=> | | |
| | | | | NADH + CO2 + 2-Oxoglutarate + H+), ATP: acetate | | |
| | | | | phosphotransferase (ATP + H+ + Propionate --> ADP + | | |
| | | | | Propionyl phosphate) | | |
| 38 | Yield- | 2 | CAETHG_3359, | ATP: acetate phosphotransferase (ATP + H+ + Propionate --> | 0.03068 | 0.013564 |
| | LMOMA | | CAETHG_3510 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <2 Oxoglutarate + LL-2,6- | | |
| | | | | Diaminopimelate) | | |
| 39 | Yield- | 2 | CAETHG_2721, | Phosphoenolpyruvate carboxykinase (PPCK) (ATP + | 0 | 0.01353 |
| | LMOMA | | CAETHG_3359 | Oxaloacetate + H+ --> ADP + CO2 + Phosphoenolpyruvate), | | |
| | | | | ATP: acetate phosphotransferase (ATP + H+ + Propionate --> | | |
| | | | | ADP + Propionyl phosphate) | | |
| 40 | Yield- | 2 | CAETHG_2753, | Isocitrate dehydrogenase (NAD + Isocitrate <=> NADH + | 0 | 0.013514 |
| | LMOMA | | CAETHG_3359 | CO2 + 2-Oxoglutarate + H+), ATP: acetate phosphotransferase | | |
| | | | | (ATP + H+ + Propionate --> ADP + Propionyl phosphate) | | |
| 41 | Yield- | 2 | CAETHG_0248, | L-lactate reversible transport via proton symport (H+_ext + | 0 | 0.011978 |
| | LMOMA | | CAETHG_2753 | L-Lactate_ext <=> H+ + L-Lactate), Isocitrate dehydrogenase | | |
| | | | | (NAD + Isocitrate <=> NADH + CO2 + 2-Oxoglutarate + H+) | | |
| 42 | Yield- | 2 | CAETHG_2753, | Isocitrate dehydrogenase (NAD + Isocitrate <=> NADH + | 0 | 0.011892 |
| | LMOMA | | CAETHG_3924 | CO2 + 2-Oxoglutarate + H+), D-Ribose 1,5-phosphomutase | | |
| | | | | (Ribose 1-phosphate <=> ribose-5-phosphate) | | |
| 43 | Yield- | 2 | CAETHG_0160, | Isocitrate dehydrogenase (NAD + Isocitrate <=> NADH + | 0 | 0.011892 |
| | LMOMA | | CAETHG_2753 | CO2 + 2-Oxoglutarate + H+) | | |
| 44 | Yield- | 2 | CAETHG_2753, | Isocitrate dehydrogenase (NAD + Isocitrate <=> NADH + | 0 | 0.011848 |
| | LMOMA | | CAETHG_0498 | CO2 + 2-Oxoglutarate + H+), Cystathionine beta synthase (L- | | |
| | | | | Serine + Homocysteine <=> H2O + Cystathionine) | | |
| 45 | Yield- | 2 | CAETHG_2753, | Isocitrate dehydrogenase (NAD + Isocitrate <=> NADH + | 0 | 0.01184 |
| | LMOMA | | CAETHG_3327 | CO2 + 2-Oxoglutarate + H+), phosphate ABC transporter | | |
| | | | | permease protein (PPi + H+ --> PPi_ext + H+_ext) | | |
| 46 | Yield- | 2 | CAETHG_2721, | Phosphoenolpyruvate carboxykinase (PPCK) (ATP + | 0 | 0.011832 |
| | LMOMA | | CAETHG_2753 | Oxaloacetate + H+ --> ADP + CO2 + Phosphoenolpyruvate), | | |
| | | | | Isocitrate dehydrogenase (NAD + Isocitrate <=> NADH + | | |
| | | | | CO2 + 2-Oxoglutarate + H+) | | |
| 47 | Yield- | 1 | CAETHG_2753 | Isocitrate dehydrogenase (NAD + Isocitrate <=> NADH + | 0 | 0.011772 |
| | LMOMA | | | CO2 + 2-Oxoglutarate + H+) | | |

### Example 9

This example describes disruptions for improved production of 2-butanol in Wood-Ljungdahl microorganisms. Metabolic modeling experiments were performed as described in Example 1. Production of 2-butanol in Wood-Ljungdahl microorganisms is described, e.g., in WO 2013/185123. The following pathway was used to model 2-butanol production herein: NADH+H++(R)-Acetoin-->NAD+meso-2,3-Butanediol; meso-2,3-Butanediol-->H2O+MEK; MEK+NADPH+H+-->2-butanol+NADP; 2-butanol-->2-butanol ext.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | Yield- | | CAETHG_2721, | Phosphoenolpyruvate carboxykinase (PPCK) (ATP + | 0 | 0.020756 |
| | LMOMA | 4 | CAETHG_3924, | Oxaloacetate + H+ --> ADP + CO2 + Phosphoenolpyruvate), | | |
| | | | CAETHG_0498, | D-Ribose 1,5-phosphomutase (Ribose 1-phosphate <=> | | |
| | | | CAETHG_0686 | ribose-5-phosphate), Cystathionine beta lyase (H2O + | | |
| | | | | Cystathionine --> NH3 + Pyruvate + Homocysteine), L- | | |
| | | | | Threonine acetaldehyde-lyase (L-Threonine --> Glycine + | | |
| | | | | Acetaldehyde) | | |
| 2 | Yield- | 4 | CAETHG_1371, | Adenosine 5′-monophosphate phosphohydrolase (H2O + | 0 | 0.020756 |
| | LMOMA | | CAETHG_2721, | AMP <=> Phosphate + H+ + Adenosine), | | |
| | | | CAETHG_0498, | Phosphoenolpyruvate carboxykinase (PPCK) (ATP + | | |
| | | | CAETHG_0686 | Oxaloacetate + H+ --> ADP + CO2 + Phosphoenolpyruvate), | | |
| | | | | Cystathionine beta lyase (H2O + Cystathionine --> NH3 + | | |
| | | | | Pyruvate + Homocysteine), L-Threonine acetaldehyde-lyase | | |
| | | | | (L-Threonine --> Glycine + Acetaldehyde) | | |
| 3 | Yield- | 4 | CAETHG_1270, | AMP: pyrophosphate phosphoribosyltransferase (PPi + AMP <=> | 0 | 0.020756 |
| | LMOMA | | CAETHG_2721, | PRPP + Adenine), Phosphoenolpyruvate carboxykinase | | |
| | | | CAETHG_0498, | (PPCK) (ATP + Oxaloacetate + H+ --> ADP + CO2 + | | |
| | | | CAETHG_0686 | Phosphoenolpyruvate), Cystathionine beta lyase (H2O + | | |
| | | | | Cystathionine --> NH3 + Pyruvate + Homocysteine), L- | | |
| | | | | Threonine acetaldehyde-lyase (L-Threonine --> Glycine + | | |
| | | | | Acetaldehyde) | | |
| 4 | Yield- | 4 | CAETHG_1371, | Adenosine 5′-monophosphate phosphohydrolase (H2O + | 0 | 0.02064 |
| | LMOMA | | CAETHG_2721, | AMP <=> Phosphate + H+ + Adenosine), | | |
| | | | CAETHG_3293, | Phosphoenolpyruvate carboxykinase (PPCK) (ATP + | | |
| | | | CAETHG_0686 | Oxaloacetate + H+ --> ADP + CO2 + Phosphoenolpyruvate), | | |
| | | | | Cysteine desulfhydrase (H2O + L-Cysteine <=> NH3 + | | |
| | | | | Pyruvate + H2S), L-Threonine acetaldehyde-lyase (L- | | |
| | | | | Threonine --> Glycine + Acetaldehyde) | | |
| 5 | Yield- | 4 | CAETHG_1270, | AMP: pyrophosphate phosphoribosyltransferase (PPi + AMP <=> | 0 | 0.02064 |
| | LMOMA | | CAETHG_2721, | PRPP + Adenine), Phosphoenolpyruvate carboxykinase | | |
| | | | CAETHG_3293, | (PPCK) (ATP + Oxaloacetate + H+ --> ADP + CO2 + | | |
| | | | CAETHG_0686 | Phosphoenolpyruvate), Cysteine desulfhydrase (H2O + L- | | |
| | | | | Cysteine <=> NH3 + Pyruvate + H2S), L-Threonine | | |
| | | | | acetaldehyde-lyase (L-Threonine --> Glycine + Acetaldehyde) | | |
| 6 | Yield- | 4 | CAETHG_1371, | Adenosine 5′-monophosphate phosphohydrolase (H2O + | 0 | 0.020476 |
| | LMOMA | | CAETHG_2721, | AMP <=> Phosphate + H+ + Adenosine), | | |
| | | | CAETHG_3021, | Phosphoenolpyruvate carboxykinase (PPCK) (ATP + | | |
| | | | CAETHG_0686 | Oxaloacetate + H+ --> ADP + CO2 + Phosphoenolpyruvate), L- | | |
| | | | | Arginine iminohydrolase (H2O + L-Arginine <=> NH3 + | | |
| | | | | Citrulline), L-Threonine acetaldehyde-lyase (L-Threonine --> | | |
| | | | | Glycine + Acetaldehyde) | | |
| 7 | Yield- | 4 | CAETHG_2721, | Phosphoenolpyruvate carboxykinase (PPCK) (ATP + | 0 | 0.020476 |
| | LMOMA | | CAETHG_3021, | Oxaloacetate + H+ --> ADP + CO2 + Phosphoenolpyruvate), L- | | |
| | | | CAETHG_3924, | Arginine iminohydrolase (H2O + L-Arginine <=> NH3 + | | |
| | | | CAETHG_0686 | Citrulline), D-Ribose 1,5-phosphomutase (Ribose 1- | | |
| | | | | phosphate <=> ribose-5-phosphate), L-Threonine | | |
| | | | | acetaldehyde-lyase (L-Threonine --> Glycine + Acetaldehyde) | | |
| 8 | Yield- | 3 | CAETHG_1371, | Adenosine 5′-monophosphate phosphohydrolase (H2O + | 0 | 0.020468 |
| | LMOMA | | CAETHG_2721, | AMP <=> Phosphate + H+ + Adenosine), | | |
| | | | CAETHG_0686 | Phosphoenolpyruvate carboxykinase (PPCK) (ATP + | | |
| | | | | Oxaloacetate + H+ --> ADP + CO2 + Phosphoenolpyruvate), L- | | |
| | | | | Threonine acetaldehyde-lyase (L-Threonine --> Glycine + | | |
| | | | | Acetaldehyde) | | |
| 9 | Yield- | 3 | CAETHG_1270, | AMP: pyrophosphate phosphoribosyltransferase (PPi + AMP <=> | 0 | 0.020468 |
| | LMOMA | | CAETHG_2721, | PRPP + Adenine), Phosphoenolpyruvate carboxykinase | | |
| | | | CAETHG_0686 | (PPCK) (ATP + Oxaloacetate + H+ --> ADP + CO2 + | | |
| | | | | Phosphoenolpyruvate), L-Threonine acetaldehyde-lyase (L- | | |
| | | | | Threonine --> Glycine + Acetaldehyde) | | |
| 10 | Yield- | 3 | CAETHG_2721, | Phosphoenolpyruvate carboxykinase (PPCK) (ATP + | 0 | 0.020468 |
| | LMOMA | | CAETHG_3924, | Oxaloacetate + H+ --> ADP + CO2 + Phosphoenolpyruvate), | | |
| | | | CAETHG_0686 | D-Ribose 1,5-phosphomutase (Ribose 1-phosphate <=> | | |
| | | | | ribose-5-phosphate), L-Threonine acetaldehyde-lyase (L- | | |
| | | | | Threonine --> Glycine + Acetaldehyde) | | |
| 11 | Yield- | 4 | CAETHG_2721, | Phosphoenolpyruvate carboxykinase (PPCK) (ATP + | 0 | 0.020756 |
| | LMOMA | | CAETHG_3924, | Oxaloacetate + H+ --> ADP + CO2 + Phosphoenolpyruvate), | | |
| | | | CAETHG_0498, | D-Ribose 1,5-phosphomutase (Ribose 1-phosphate <=> | | |
| | | | CAETHG_0686 | ribose-5-phosphate), Cystathionine beta lyase (H2O + | | |
| | | | | Cystathionine --> NH3 + Pyruvate + Homocysteine), L- | | |
| | | | | Threonine acetaldehyde-lyase (L-Threonine --> Glycine + | | |
| | | | | Acetaldehyde) | | |

### Example 10

This example describes disruptions for improved production of 2-hydroxyisobutyric acid in Wood-Ljungdahl microorganisms. Metabolic modeling experiments were performed as described in Example 1. Production of 2-hydroxyisobutyric acid in Wood-Ljungdahl microorganisms is described, e.g., in WO 2017/0066498.

The following pathway was used to model 2-hydroxyisobutyric acid production in rows 1-40 below: 2.0 Acetyl-CoA-->CoA+Acetoacetyl-CoA; NADH+H++Acetoacetyl-CoA-->NAD+(S)-3-Hydroxybutyryl-CoA; (S)-3-Hydroxybutyryl-CoA-->2HIB-CoA; 2HIB-CoA+H₂O-->2hib+CoA; 2hib-->2hib_ext.

The following pathway was used to model 2-hydroxyisobutyric acid production in rows 41-93 below: 2.0 Acetyl-CoA-->CoA+Acetoacetyl-CoA; NADH+H++Acetoacetyl-CoA-->NAD+(S)-3-Hydroxybutyryl-CoA; (S)-3-Hydroxybutyryl-CoA-->2HIB-CoA; 2HIB-CoA+ADP+Phosphate-->2hib+ATP+CoA; 2hib-->2hib_ext.

The following pathway was used to model 2-hydroxyisobutyric acid production in rows 94-103 below: 2.0 Acetyl-CoA-->CoA+Acetoacetyl-CoA; NADH+H++Acetoacetyl-CoA-->NAD+(S)-3-Hydroxybutyryl-CoA; (S)-3-Hydroxybutyryl-CoA-->2HIB-CoA; 2HIB-CoA+Acetate-->2hib+Acetyl-CoA; 2hib-->2hib_ext.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | Yield- | 4 | CAETHG_2721, | Phosphoenolpyruvate carboxykinase (PPCK) (ATP + | 0 | 0.074552 |
| | LMOMA | | CAETHG_2932, | Oxaloacetate + H+ --> ADP + CO2 + | | |
| | | | CAETHG_3359, | Phosphoenolpyruvate), Alpha-acetolactate decarboxylase | | |
| | | | CAETHG_0498 | (ALCTT --> CO2 + (R)-Acetoin), ATP: acetate | | |
| | | | | phosphotransferase (ATP + H+ + Propionate --> ADP + | | |
| | | | | Propionyl phosphate), Cystathionine beta lyase (H2O + | | |
| | | | | Cystathionine --> NH3 + Pyruvate + Homocysteine) | | |
| 2 | Yield- | 4 | CAETHG_2721, | Phosphoenolpyruvate carboxykinase (PPCK) (ATP + | 0 | 0.074552 |
| | LMOMA | | CAETHG_2932, | Oxaloacetate + H+ --> ADP + CO2 + | | |
| | | | CAETHG_3358, | Phosphoenolpyruvate), Alpha-acetolactate decarboxylase | | |
| | | | CAETHG_0498 | (ALCTT --> CO2 + (R)-Acetoin), Phosphate transacetylase | | |
| | | | | (Phosphate + Acetyl-CoA + H+ <=> CoA + | | |
| | | | | Acetylphosphate), Cystathionine beta lyase (H2O + | | |
| | | | | Cystathionine --> NH3 + Pyruvate + Homocysteine) | | |
| 3 | Yield- | 3 | CAETHG_2721, | Phosphoenolpyruvate carboxykinase (PPCK) (ATP + | 0 | 0.071296 |
| | LMOMA | | CAETHG_2932, | Oxaloacetate + H+ --> ADP + CO2 + | | |
| | | | CAETHG_3358 | Phosphoenolpyruvate), Alpha-acetolactate decarboxylase | | |
| | | | | (ALCTT --> CO2 + (R)-Acetoin), Phosphate transacetylase | | |
| | | | | (Phosphate + Acetyl-CoA + H+ <=> CoA + | | |
| | | | | Acetylphosphate) | | |
| 4 | Yield- | 4 | CAETHG_0160, | N-Ribosylnicotinamide: orthophosphate | 0 | 0.05776 |
| | LMOMA | | CAETHG_2932, | ribosyltransferase (Phosphate + N-Ribosylnicotinamide <=> | | |
| | | | CAETHG_3358, | Nicotinamide + Ribose 1-phosphate), Alpha- | | |
| | | | CAETHG_0498 | acetolactate decarboxylase (ALCTT --> CO2 + (R)-Acetoin), | | |
| | | | | Phosphate transacetylase (Phosphate + Acetyl-CoA + H+ <=> | | |
| | | | | CoA + Acetylphosphate), Cystathionine beta lyase | | |
| | | | | (H2O + Cystathionine --> NH3 + Pyruvate + | | |
| | | | | Homocysteine) | | |
| 5 | Yield- | 4 | CAETHG_2475, | dGTP triphosphohydrolase (H2O + dGTP --> H+ + | 0 | 0.055144 |
| | LMOMA | | CAETHG_2932, | Deoxyguanosine + Triphosphate), Alpha-acetolactate | | |
| | | | CAETHG_3358, | decarboxylase (ALCTT --> CO2 + (R)-Acetoin), Phosphate | | |
| | | | CAETHG_0498 | transacetylase (Phosphate + Acetyl-CoA + H+ <=> CoA + | | |
| | | | | Acetylphosphate), Cystathionine beta lyase (H2O + | | |
| | | | | Cystathionine --> NH3 + Pyruvate + Homocysteine) | | |
| 6 | Yield- | 3 | CAETHG_2932, | Alpha-acetolactate decarboxylase (ALCTT --> CO2 + (R)- | 0 | 0.055008 |
| | LMOMA | | CAETHG_3358, | Acetoin), Phosphate transacetylase (Phosphate + Acetyl- | | |
| | | | CAETHG_0686 | CoA + H+ <=> CoA + Acetylphosphate), L-Threonine | | |
| | | | | acetaldehyde-lyase (L-Threonine --> Glycine + | | |
| | | | | Acetaldehyde) | | |
| 7 | Yield- | 3 | CAETHG_2932, | Alpha-acetolactate decarboxylase (ALCTT --> CO2 + (R)- | 0 | 0.054724 |
| | LMOMA | | CAETHG_3358, | Acetoin), Phosphate transacetylase (Phosphate + Acetyl- | | |
| | | | CAETHG_0498 | CoA + H+ <=> CoA + Acetylphosphate), Cystathionine beta | | |
| | | | | lyase (H2O + Cystathionine --> NH3 + Pyruvate + | | |
| | | | | Homocysteine) | | |
| 8 | Yield- | 4 | CAETHG_2107, | Potassium uptake (K+_ext <=> K+), Alpha-acetolactate | 0 | 0.054364 |
| | LMOMA | | CAETHG_2932, | decarboxylase (ALCTT --> CO2 + (R)-Acetoin), Phosphate | | |
| | | | CAETHG_3358, | transacetylase (Phosphate + Acetyl-CoA + H+ <=> CoA + | | |
| | | | CAETHG_3924 | Acetylphosphate), D-Ribose 1,5-phosphomutase (Ribose | | |
| | | | | 1-phosphate <=> ribose-5-phosphate) | | |
| 9 | Yield- | 4 | CAETHG_1371, | Adenosine 5′-monophosphate phosphohydrolase (H2O + | 0 | 0.054364 |
| | LMOMA | | CAETHG_2107, | AMP <=> Phosphate + H+ + Adenosine), Potassium | | |
| | | | CAETHG_2932, | uptake (K+_ext <=> K+), Alpha-acetolactate | | |
| | | | CAETHG_3358 | decarboxylase (ALCTT --> CO2 + (R)-Acetoin), Phosphate | | |
| | | | | transacetylase (Phosphate + Acetyl-CoA + H+ <=> CoA + | | |
| | | | | Acetylphosphate) | | |
| 10 | Yield- | 3 | CAETHG_2932, | Alpha-acetolactate decarboxylase (ALCTT --> CO2 + (R)- | 0 | 0.05436 |
| | LMOMA | | CAETHG_3358, | Acetoin), Phosphate transacetylase (Phosphate + Acetyl- | | |
| | | | CAETHG_3924 | CoA + H+ <=> CoA + Acetylphosphate), D-Ribose 1,5- | | |
| | | | | phosphomutase (Ribose 1-phosphate <=> ribose-5- | | |
| | | | | phosphate) | | |
| 11 | Yield- | 3 | CAETHG_1270, | AMP: pyrophosphate phosphoribosyltransferase (PPi + | 0 | 0.05436 |
| | LMOMA | | CAETHG_2932, | AMP <=> PRPP + Adenine), Alpha-acetolactate | | |
| | | | CAETHG_3358 | decarboxylase (ALCTT --> CO2 + (R)-Acetoin), Phosphate | | |
| | | | | transacetylase (Phosphate + Acetyl-CoA + H+ <=> CoA + | | |
| | | | | Acetylphosphate) | | |
| 12 | Yield- | 3 | CAETHG_1371, | Adenosine 5′-monophosphate phosphohydrolase (H2O + | 0 | 0.05436 |
| | LMOMA | | CAETHG_2932, | AMP <=> Phosphate + H+ + Adenosine), Alpha- | | |
| | | | CAETHG_3358 | acetolactate decarboxylase (ALCTT --> CO2 + (R)-Acetoin), | | |
| | | | | Phosphate transacetylase (Phosphate + Acetyl-CoA + H+ <=> | | |
| | | | | CoA + Acetylphosphate) | | |
| 13 | Yield- | 3 | CAETHG_0160, | N-Ribosylnicotinamide: orthophosphate | 0 | 0.05436 |
| | LMOMA | | CAETHG_2932, | ribosyltransferase (Phosphate + N-Ribosylnicotinamide <=> | | |
| | | | CAETHG_3358 | Nicotinamide + Ribose 1-phosphate), Alpha- | | |
| | | | | acetolactate decarboxylase (ALCTT --> CO2 + (R)-Acetoin), | | |
| | | | | Phosphate transacetylase (Phosphate + Acetyl-CoA + H+ <=> | | |
| | | | | CoA + Acetylphosphate) | | |
| 14 | Yield- | 3 | CAETHG_2932, | Alpha-acetolactate decarboxylase (ALCTT --> CO2 + (R)- | 0 | 0.051804 |
| | LMOMA | | CAETHG_3299, | Acetoin), 2-Deoxy-D-ribose-5-phosphate acetaldehyde- | | |
| | | | CAETHG_3358 | lyase (deoxyribose-5-phosphate <=> Acetaldehyde + | | |
| | | | | Glyceraldehyde3-phosphate), Phosphate transacetylase | | |
| | | | | (Phosphate + Acetyl-CoA + H+ <=> CoA + | | |
| | | | | Acetylphosphate) | | |
| 15 | Yield- | 3 | CAETHG_2475, | dGTP triphosphohydrolase (H2O + dGTP --> H+ + | 0 | 0.051796 |
| | LMOMA | | CAETHG_2932, | Deoxyguanosine + Triphosphate), Alpha-acetolactate | | |
| | | | CAETHG_3358 | decarboxylase (ALCTT --> CO2 + (R)-Acetoin), Phosphate | | |
| | | | | transacetylase (Phosphate + Acetyl-CoA + H+ <=> CoA + | | |
| | | | | Acetylphosphate) | | |
| 16 | Yield- | 3 | CAETHG_2932, | Alpha-acetolactate decarboxylase (ALCTT --> CO2 + (R)- | 0 | 0.051492 |
| | LMOMA | | CAETHG_3021, | Acetoin), L-Arginine iminohydrolase (H2O + L-Arginine <=> | | |
| | | | CAETHG_3358 | NH3 + Citrulline), Phosphate transacetylase | | |
| | | | | (Phosphate + Acetyl-CoA + H+ <=> CoA + | | |
| | | | | Acetylphosphate) | | |
| 17 | Yield- | 4 | CAETHG_2107, | Potassium uptake (K+_ext <=> K+), Alpha-acetolactate | 0 | 0.051376 |
| | LMOMA | | CAETHG_2932, | decarboxylase (ALCTT --> CO2 + (R)-Acetoin), | | |
| | | | | UMP: pyrophosphate phosphoribosyltransferase (Uracil + | | |
| | | | CAETHG_3164, | PRPP --> PPi + UMP), Phosphate transacetylase | | |
| | | | CAETHG_3358 | (Phosphate + Acetyl-CoA + H+ <=> CoA + | | |
| | | | | Acetylphosphate) | | |
| 18 | Yield- | 3 | CAETHG_2107, | Potassium uptake (K+_ext <=> K+), Alpha-acetolactate | 0 | 0.051368 |
| | LMOMA | | CAETHG_2932, | decarboxylase (ALCTT --> CO2 + (R)-Acetoin), Phosphate | | |
| | | | CAETHG_3358 | transacetylase (Phosphate + Acetyl-CoA + H+ <=> CoA + | | |
| | | | | Acetylphosphate) | | |
| 19 | Yield- | 2 | CAETHG_2932, | Alpha-acetolactate decarboxylase (ALCTT --> CO2 + (R)- | 0 | 0.051364 |
| | LMOMA | | CAETHG_3358 | Acetoin), Phosphate transacetylase (Phosphate + Acetyl- | | |
| | | | | CoA + H+ <=> CoA + Acetylphosphate) | | |
| 20 | Yield- | 2 | CAETHG_2932, | Alpha-acetolactate decarboxylase (ALCTT --> CO2 + (R)- | 0 | 0.051364 |
| | LMOMA | | CAETHG_3359 | Acetoin), ATP: acetate phosphotransferase (ATP + H+ + | | |
| | | | | Propionate --> ADP + Propionyl phosphate) | | |
| 21 | Yield- | 3 | CAETHG_2751, | Citramalate synthase (H2O + Pyruvate + Acetyl-CoA <=> | 0 | 0.047452 |
| | LMOMA | | CAETHG_3358, | CoA + Citramalate), Phosphate transacetylase (Phosphate + | | |
| | | | CAETHG_0498 | Acetyl-CoA + H+ <=> CoA + Acetylphosphate), | | |
| | | | | Cystathionine beta lyase (H2O + Cystathionine --> NH3 + | | |
| | | | | Pyruvate + Homocysteine) | | |
| 22 | Yield- | 3 | CAETHG_3021, | L-Arginine iminohydrolase (H2O + L-Arginine <=> NH3 + | 0 | 0.045856 |
| | LMOMA | | CAETHG_3358, | Citrulline), Phosphate transacetylase (Phosphate + Acetyl- | | |
| | | | CAETHG_0498 | CoA + H+ <=> CoA + Acetylphosphate), Cystathionine beta | | |
| | | | | lyase (H2O + Cystathionine --> NH3 + Pyruvate + | | |
| | | | | Homocysteine) | | |
| 23 | Yield- | 3 | CAETHG_2475, | dGTP triphosphohydrolase (H2O + dGTP --> H+ + | 0 | 0.04582 |
| | LMOMA | | CAETHG_3358, | Deoxyguanosine + Triphosphate), Phosphate | | |
| | | | CAETHG_0498 | transacetylase (Phosphate + Acetyl-CoA + H+ <=> CoA + | | |
| | | | | Acetylphosphate), Cystathionine beta lyase (H2O + | | |
| | | | | Cystathionine --> NH3 + Pyruvate + Homocysteine) | | |
| 24 | Yield- | 2 | CAETHG_3359, | ATP: acetate phosphotransferase (ATP + H+ + Propionate--> | 0 | 0.045768 |
| | LMOMA | | CAETHG_0498 | ADP + Propionyl phosphate), Cystathionine beta lyase | | |
| | | | | (H2O + Cystathionine --> NH3 + Pyruvate + | | |
| | | | | Homocysteine) | | |
| 25 | Yield- | 2 | CAETHG_3358, | Phosphate transacetylase (Phosphate + Acetyl-CoA + H+ <=> | 0 | 0.045768 |
| | LMOMA | | CAETHG_0498 | CoA + Acetylphosphate), Cystathionine beta lyase | | |
| | | | | (H2O + Cystathionine --> NH3 + Pyruvate + | | |
| | | | | Homocysteine) | | |
| 26 | Yield- | 2 | CAETHG_3359, | ATP: acetate phosphotransferase (ATP + H+ + Propionate--> | 0 | 0.04472 |
| | LMOMA | | CAETHG_3924 | ADP + Propionyl phosphate), D-Ribose 1,5- | | |
| | | | | phosphomutase (Ribose 1-phosphate <=> ribose-5- | | |
| | | | | phosphate) | | |
| 27 | Yield- | 2 | CAETHG_1371, | Adenosine 5′-monophosphate phosphohydrolase (H2O + | 0 | 0.04472 |
| | LMOMA | | CAETHG_3359 | AMP <=> Phosphate + H+ + Adenosine), ATP: acetate | | |
| | | | | phosphotransferase (ATP + H+ + Propionate --> ADP + | | |
| | | | | Propionyl phosphate) | | |
| 28 | Yield- | 2 | CAETHG_1270, | AMP: pyrophosphate phosphoribosyltransferase (PPi + | 0 | 0.04472 |
| | LMOMA | | CAETHG_3359 | AMP <=> PRPP + Adenine), ATP: acetate | | |
| | | | | phosphotransferase (ATP + H+ + Propionate --> ADP + | | |
| | | | | Propionyl phosphate) | | |
| 29 | Yield- | 2 | CAETHG_0160, | N-Ribosylnicotinamide: orthophosphate | 0 | 0.04472 |
| | LMOMA | | CAETHG_3359 | ribosyltransferase (Phosphate + N-Ribosylnicotinamide <=> | | |
| | | | | Nicotinamide + Ribose 1-phosphate), ATP: acetate | | |
| | | | | phosphotransferase (ATP + H+ + Propionate --> ADP + | | |
| | | | | Propionyl phosphate) | | |
| 30 | Yield- | 2 | CAETHG_3359, | ATP: acetate phosphotransferase (ATP + H+ + Propionate--> | 0 | 0.04366 |
| | LMOMA | | CAETHG_0686 | ADP + Propionyl phosphate), L-Threonine | | |
| | | | | acetaldehyde-lyase (L-Threonine --> Glycine + | | |
| | | | | Acetaldehyde) | | |
| 31 | Yield- | 2 | CAETHG_3021, | L-Arginine iminohydrolase (H2O + L-Arginine <=> NH3 + | 0 | 0.043384 |
| | LMOMA | | CAETHG_3359 | Citrulline), ATP: acetate phosphotransferase (ATP + H+ + | | |
| | | | | Propionate --> ADP + Propionyl phosphate) | | |
| 32 | Yield- | 2 | CAETHG_2107, | Potassium uptake (K+_ext <=> K+), ATP: acetate | 0 | 0.0433 |
| | LMOMA | | CAETHG_3359 | phosphotransferase (ATP + H+ + Propionate --> ADP + | | |
| | | | | Propionyl phosphate) | | |
| 33 | Yield- | 1 | CAETHG_3358 | Phosphate transacetylase (Phosphate + Acetyl-CoA + H+ <=> | 0 | 0.043296 |
| | LMOMA | | | CoA + Acetylphosphate) | | |
| 34 | Yield- | 1 | CAETHG_3359 | ATP: acetate phosphotransferase (ATP + H+ + Propionate--> | 0 | 0.043296 |
| | LMOMA | | | ADP + Propionyl phosphate) | | |
| 35 | Yield- | 1 | CAETHG_1371 | Adenosine 5′-monophosphate phosphohydrolase (H2O + | 0 | 0.020432 |
| | LMOMA | | | AMP <=> Phosphate + H+ + Adenosine) | | |
| 36 | Yield- | 1 | CAETHG_0160 | N-Ribosylnicotinamide: orthophosphate | 0 | 0.020432 |
| | LMOMA | | | ribosyltransferase (Phosphate + N-Ribosylnicotinamide <=> | | |
| | | | | Nicotinamide + Ribose 1-phosphate) | | |
| 37 | Yield- | 1 | CAETHG_1270 | AMP: pyrophosphate phosphoribosyltransferase (PPi + | 0 | 0.020432 |
| | LMOMA | | | AMP <=> PRPP + Adenine) | | |
| 38 | Yield- | 1 | CAETHG_3924 | D-Ribose 1,5-phosphomutase (Ribose 1-phosphate <=> | 0 | 0.020432 |
| | LMOMA | | | ribose-5-phosphate) | | |
| 39 | Yield- | 1 | CAETHG_2721 | Phosphoenolpyruvate carboxykinase (PPCK) (ATP + | 0 | 0.020356 |
| | LMOMA | | | Oxaloacetate + H+ --> ADP + CO2 + | | |
| | | | | Phosphoenolpyruvate) | | |
| 40 | Yield- | 1 | CAETHG_3021 | L-Arginine iminohydrolase (H2O + L-Arginine <=> NH3 + | 0 | 0.020028 |
| | LMOMA | | | Citrulline) | | |
| 41 | BPCY- | 1 | CAETHG_3358 | Phosphate transacetylase (Phosphate + Acetyl-CoA + H+ <=> | 0.21671 | 0.048129 |
| | FBA | | | CoA + Acetylphosphate) | | |
| 42 | BPCY- | 1 | CAETHG_3359 | ATP: acetate phosphotransferase (ATP + H+ + Propionate--> | 0.21671 | 0.048129 |
| | FBA | | | ADP + Propionyl phosphate) | | |
| 43 | BPCY- | 1 | CAETHG_2909 | ATP: pyruvate, orthophosphate phosphotransferase (ATP + | 0.21241 | 0.000573 |
| | FBA | | | Phosphate + Pyruvate + H+ --> PPi + AMP + | | |
| | | | | Phosphoenolpyruvate) | | |
| 44 | Yield- | 4 | CAETHG_0234, | 4,5-Dihydro-4-oxo-5-imidazolepropanoate hydro-lyase (4- | 0.21241 | 0.11296 |
| | LMOMA | | CAETHG_2751, | Imidazolone-5-propanoate <=> H2O + Urocanate), | | |
| | | | CAETHG_3359, | Citramalate synthase (H2O + Pyruvate + Acetyl-CoA <=> | | |
| | | | CAETHG_3510 | CoA + Citramalate), ATP: acetate phosphotransferase (ATP + | | |
| | | | | H+ + Propionate --> ADP + Propionyl phosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 45 | Yield- | 4 | CAETHG_0233, | 4-imidazolone-5-propanoate amidohydrolase (H2O + 4- | 0.21058 | 0.11296 |
| | LMOMA | | CAETHG_2751, | Imidazolone-5-propanoate --> N-Formimino-L- | | |
| | | | CAETHG_3359, | glutamate), Citramalate synthase (H2O + Pyruvate + | | |
| | | | CAETHG_3510 | Acetyl-CoA <=> CoA + Citramalate), ATP: acetate | | |
| | | | | phosphotransferase (ATP + H+ + Propionate --> ADP + | | |
| | | | | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 46 | Yield- | 2 | CAETHG_3358, | Phosphate transacetylase (Phosphate + Acetyl-CoA + H+ <=> | 0.21058 | 0.111576 |
| | LMOMA | | CAETHG_3510 | CoA + Acetylphosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 47 | Yield- | 2 | CAETHG_3359, | ATP: acetate phosphotransferase (ATP + H+ + Propionate--> | 0.21544 | 0.111576 |
| | LMOMA | | CAETHG_3510 | ADP + Propionyl phosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 48 | Yield- | 4 | CAETHG_2721, | Phosphoenolpyruvate carboxykinase (PPCK) (ATP + | 0.21528 | 0.104448 |
| | LMOMA | | CAETHG_2753, | Oxaloacetate + H+ --> ADP + CO2 + | | |
| | | | CAETHG_3358, | Phosphoenolpyruvate), Isocitrate dehydrogenase (NAD + | | |
| | | | CAETHG_0498 | Isocitrate <=> NADH + CO2 + 2-Oxoglutarate + H+), | | |
| | | | | Phosphate transacetylase (Phosphate + Acetyl-CoA + H+ <=> | | |
| | | | | CoA + Acetylphosphate), Cystathionine beta lyase | | |
| | | | | (H2O + Cystathionine --> NH3 + Pyruvate + | | |
| | | | | Homocysteine) | | |
| 49 | Yield- | 3 | CAETHG_2721, | Phosphoenolpyruvate carboxykinase (PPCK) (ATP + | 0.21528 | 0.104424 |
| | LMOMA | | CAETHG_3358, | Oxaloacetate + H+ --> ADP + CO2 + | | |
| | | | CAETHG_0498 | Phosphoenolpyruvate), Phosphate transacetylase | | |
| | | | | (Phosphate + Acetyl-CoA + H+ <=> CoA + | | |
| | | | | Acetylphosphate), Cystathionine beta lyase (H2O + | | |
| | | | | Cystathionine --> NH3 + Pyruvate + Homocysteine) | | |
| 50 | Yield- | 4 | CAETHG_2721, | Phosphoenolpyruvate carboxykinase (PPCK) (ATP + | 0.21528 | 0.102516 |
| | LMOMA | | CAETHG_3021, | Oxaloacetate + H+ --> ADP + CO2 + | | |
| | | | CAETHG_3358, | Phosphoenolpyruvate), L-Arginine iminohydrolase (H2O + | | |
| | | | CAETHG_3924 | L-Arginine <=> NH3 + Citrulline), Phosphate | | |
| | | | | transacetylase (Phosphate + Acetyl-CoA + H+ <=> CoA + | | |
| | | | | Acetylphosphate), D-Ribose 1,5-phosphomutase (Ribose | | |
| | | | | 1-phosphate <=> ribose-5-phosphate) | | |
| 51 | Yield- | 3 | CAETHG_1371, | Adenosine 5′-monophosphate phosphohydrolase (H2O + | 0.21221 | 0.102476 |
| | LMOMA | | CAETHG_2721, | AMP <=> Phosphate + H+ + Adenosine), | | |
| | | | | CAETHG_3358 Phosphoenolpyruvate | | |
| | | | | carboxykinase (PPCK) (ATP + | | |
| | | | | Oxaloacetate + H+ --> ADP + CO2 + | | |
| | | | | Phosphoenolpyruvate), Phosphate transacetylase | | |
| | | | | (Phosphate + Acetyl-CoA + H+ <=> CoA + | | |
| | | | | Acetylphosphate) | | |
| 52 | Yield- | 3 | CAETHG_2721, | Phosphoenolpyruvate carboxykinase (PPCK) (ATP + | 0.21204 | 0.102476 |
| | LMOMA | | CAETHG_3358, | Oxaloacetate + H+ --> ADP + CO2 + | | |
| | | | CAETHG_3924 | Phosphoenolpyruvate), Phosphate transacetylase | | |
| | | | | (Phosphate + Acetyl-CoA + H+ <=> CoA + | | |
| | | | | Acetylphosphate), D-Ribose 1,5-phosphomutase (Ribose | | |
| | | | | 1-phosphate <=> ribose-5-phosphate) | | |
| 53 | Yield- | 3 | CAETHG_1270, | AMP: pyrophosphate phosphoribosyltransferase (PPi + | 0.21204 | 0.102464 |
| | LMOMA | | CAETHG_2721, | AMP <=> PRPP + Adenine), Phosphoenolpyruvate | | |
| | | | CAETHG_3358 | carboxykinase (PPCK) (ATP + Oxaloacetate + H+ --> ADP + | | |
| | | | | CO2 + Phosphoenolpyruvate), Phosphate transacetylase | | |
| | | | | (Phosphate + Acetyl-CoA + H+ <=> CoA + | | |
| | | | | Acetylphosphate) | | |
| 54 | Yield- | 3 | CAETHG_2721, | Phosphoenolpyruvate carboxykinase (PPCK) (ATP + | 0.21204 | 0.102348 |
| | LMOMA | | CAETHG_3021, | Oxaloacetate + H+ --> ADP + CO2 + | | |
| | | | CAETHG_3358 | Phosphoenolpyruvate), L-Arginine iminohydrolase (H2O + | | |
| | | | | L-Arginine <=> NH3 + Citrulline), Phosphate | | |
| | | | | transacetylase (Phosphate + Acetyl-CoA + H+ <=> CoA + | | |
| | | | | Acetylphosphate) | | |
| 55 | Yield- | 3 | CAETHG_2721, | Phosphoenolpyruvate carboxykinase (PPCK) (ATP + | 0.21204 | 0.102324 |
| | LMOMA | | CAETHG_2753, | Oxaloacetate + H+ --> ADP + CO2 + | | |
| | | | CAETHG_3358 | Phosphoenolpyruvate), Isocitrate dehydrogenase (NAD + | | |
| | | | | Isocitrate <=> NADH + CO2 + 2-Oxoglutarate + H+), | | |
| | | | | Phosphate transacetylase (Phosphate + Acetyl-CoA + H+ <=> | | |
| | | | | CoA + Acetylphosphate) | | |
| 56 | Yield- | 3 | CAETHG_2107, | Potassium uptake (K+_ext <=> K+), Phosphoenolpyruvate | 0.21353 | 0.102304 |
| | LMOMA | | CAETHG_2721, | carboxykinase (PPCK) (ATP + Oxaloacetate + H+ --> ADP + | | |
| | | | CAETHG_3358 | CO2 + Phosphoenolpyruvate), Phosphate transacetylase | | |
| | | | | (Phosphate + Acetyl-CoA + H+ <=> CoA + | | |
| | | | | Acetylphosphate) | | |
| 57 | Yield- | 2 | CAETHG_2721, | Phosphoenolpyruvate carboxykinase (PPCK) (ATP + | 0.21353 | 0.1023 |
| | LMOMA | | CAETHG_3358 | Oxaloacetate + H+ --> ADP + CO2 + | | |
| | | | | Phosphoenolpyruvate), Phosphate transacetylase | | |
| | | | | (Phosphate + Acetyl-CoA + H+ <=> CoA + | | |
| | | | | Acetylphosphate) | | |
| 58 | Yield- | 2 | CAETHG_2721, | Phosphoenolpyruvate carboxykinase (PPCK) (ATP + | 0.21241 | 0.1023 |
| | LMOMA | | CAETHG_3359 | Oxaloacetate + H+ --> ADP + CO2 + | | |
| | | | | Phosphoenolpyruvate), ATP: acetate phosphotransferase | | |
| | | | | (ATP + H+ + Propionate --> ADP + Propionyl phosphate) | | |
| 59 | Yield- | 3 | CAETHG_1371, | Adenosine 5′-monophosphate phosphohydrolase (H2O + | 0.21241 | 0.090748 |
| | LMOMA | | CAETHG_2932, | AMP <=> Phosphate + H+ + Adenosine), Alpha- | | |
| | | | CAETHG_3358 | acetolactate decarboxylase (ALCTT --> CO2 + (R)-Acetoin), | | |
| | | | | Phosphate transacetylase (Phosphate + Acetyl-CoA + H+ <=> | | |
| | | | | CoA + Acetylphosphate) | | |
| 60 | Yield- | 3 | CAETHG_1270, | AMP: pyrophosphate phosphoribosyltransferase (PPi + | 0.21171 | 0.090656 |
| | LMOMA | | CAETHG_2932, | AMP <=> PRPP + Adenine), Alpha-acetolactate | | |
| | | | CAETHG_3358 | decarboxylase (ALCTT --> CO2 + (R)-Acetoin), Phosphate | | |
| | | | | transacetylase (Phosphate + Acetyl-CoA + H+ <=> CoA + | | |
| | | | | Acetylphosphate) | | |
| 61 | Yield- | 2 | CAETHG_2932, | Alpha-acetolactate decarboxylase (ALCTT --> CO2 + (R)- | 0.21171 | 0.089548 |
| | LMOMA | | CAETHG_3359 | Acetoin), ATP: acetate phosphotransferase (ATP + H+ + | | |
| | | | | Propionate --> ADP + Propionyl phosphate) | | |
| 62 | Yield- | 2 | CAETHG_2932, | Alpha-acetolactate decarboxylase (ALCTT --> CO2 + (R)- | 0.21171 | 0.089548 |
| | LMOMA | | CAETHG_3358 | Acetoin), Phosphate transacetylase (Phosphate + Acetyl- | | |
| | | | | CoA + H+ <=> CoA + Acetylphosphate) | | |
| 63 | Yield- | 3 | CAETHG_2475, | dGTP triphosphohydrolase (H2O + dGTP --> H+ + | 0.21646 | 0.083936 |
| | LMOMA | | CAETHG_3358, | Deoxyguanosine + Triphosphate), Phosphate | | |
| | | | CAETHG_0686 | transacetylase (Phosphate + Acetyl-CoA + H+ <=> CoA + | | |
| | | | | Acetylphosphate), L-Threonine acetaldehyde-lyase (L- | | |
| | | | | Threonine --> Glycine + Acetaldehyde) | | |
| 64 | Yield- | 2 | CAETHG_3359, | ATP: acetate phosphotransferase (ATP + H+ + Propionate--> | 0.21753 | 0.083768 |
| | LMOMA | | CAETHG_0686 | ADP + Propionyl phosphate), L-Threonine | | |
| | | | | acetaldehyde-lyase (L-Threonine --> Glycine + | | |
| | | | | Acetaldehyde) | | |
| 65 | Yield- | 2 | CAETHG_3358, | Phosphate transacetylase (Phosphate + Acetyl-CoA + H+ <=> | 0.21753 | 0.083768 |
| | LMOMA | | CAETHG_0686 | CoA + Acetylphosphate), L-Threonine acetaldehyde- | | |
| | | | | lyase (L-Threonine --> Glycine + Acetaldehyde) | | |
| 66 | Yield- | 3 | CAETHG_0160, | N-Ribosylnicotinamide: orthophosphate | 0.21753 | 0.082284 |
| | LMOMA | | CAETHG_3358, | ribosyltransferase (Phosphate + N-Ribosylnicotinamide <=> | | |
| | | | CAETHG_0498 | Nicotinamide + Ribose 1-phosphate), Phosphate | | |
| | | | | transacetylase (Phosphate + Acetyl-CoA + H+ <=> CoA + | | |
| | | | | Acetylphosphate), Cystathionine beta lyase (H2O + | | |
| | | | | Cystathionine --> NH3 + Pyruvate + Homocysteine) | | |
| 67 | Yield- | 3 | CAETHG_2475, | dGTP triphosphohydrolase (H2O + dGTP --> H+ + | 0.21753 | 0.080496 |
| | LMOMA | | CAETHG_3358, | Deoxyguanosine + Triphosphate), Phosphate | | |
| | | | CAETHG_0498 | transacetylase (Phosphate + Acetyl-CoA + H+ <=> CoA + | | |
| | | | | Acetylphosphate), Cystathionine beta lyase (H2O + | | |
| | | | | Cystathionine --> NH3 + Pyruvate + Homocysteine) | | |
| 68 | Yield- | 3 | CAETHG_2475, | dGTP triphosphohydrolase (H2O + dGTP --> H+ + | 0.21353 | 0.080496 |
| | LMOMA | | CAETHG_3359, | Deoxyguanosine + Triphosphate), ATP: acetate | | |
| | | | CAETHG_0498 | phosphotransferase (ATP + H+ + Propionate --> ADP + | | |
| | | | | Propionyl phosphate), Cystathionine beta lyase (H2O + | | |
| | | | | Cystathionine --> NH3 + Pyruvate + Homocysteine) | | |
| 69 | Yield- | 2 | CAETHG_3359, | ATP: acetate phosphotransferase (ATP + H+ + Propionate--> | 0.21353 | 0.080204 |
| | LMOMA | | CAETHG_0498 | ADP + Propionyl phosphate), Cystathionine beta lyase | | |
| | | | | (H2O + Cystathionine --> NH3 + Pyruvate + | | |
| | | | | Homocysteine) | | |
| 70 | Yield- | 2 | CAETHG_3358, | Phosphate transacetylase (Phosphate + Acetyl-CoA + H+ <=> | 0.21353 | 0.080204 |
| | LMOMA | | CAETHG_0498 | CoA + Acetylphosphate), Cystathionine beta lyase | | |
| | | | | (H2O + Cystathionine --> NH3 + Pyruvate + | | |
| | | | | Homocysteine) | | |
| 71 | Yield- | 2 | CAETHG_0160, | N-Ribosylnicotinamide: orthophosphate | 0.21353 | 0.077112 |
| | LMOMA | | CAETHG_3359 | ribosyltransferase (Phosphate + N-Ribosylnicotinamide <=> | | |
| | | | | Nicotinamide + Ribose 1-phosphate), ATP: acetate | | |
| | | | | phosphotransferase (ATP + H+ + Propionate --> ADP + | | |
| | | | | Propionyl phosphate) | | |
| 72 | Yield- | 2 | CAETHG_1371, | Adenosine 5′-monophosphate phosphohydrolase (H2O + | 0.21241 | 0.077112 |
| | LMOMA | | CAETHG_3359 | AMP <=> Phosphate + H+ + Adenosine), ATP: acetate | | |
| | | | | phosphotransferase (ATP + H+ + Propionate --> ADP + | | |
| | | | | Propionyl phosphate) | | |
| 73 | Yield- | 2 | CAETHG_3359, | ATP: acetate phosphotransferase (ATP + H+ + Propionate- | 0.21236 | 0.077112 |
| | LMOMA | | CAETHG_3924 | -> ADP + Propionyl phosphate), D-Ribose 1,5- | | |
| | | | | phosphomutase (Ribose 1-phosphate <=> ribose-5- | | |
| | | | | phosphate) | | |
| 74 | Yield- | 2 | CAETHG_1270, | AMP: pyrophosphate phosphoribosyltransferase (PPi + | 0.21241 | 0.077028 |
| | LMOMA | | CAETHG_3359 | AMP <=> PRPP + Adenine), ATP: acetate | | |
| | | | | phosphotransferase (ATP + H+ + Propionate --> ADP + | | |
| | | | | Propionyl phosphate) | | |
| 75 | Yield- | 2 | CAETHG_3299, | 2-Deoxy-D-ribose-5-phosphate acetaldehyde-lyase | 0.21241 | 0.075868 |
| | LMOMA | | CAETHG_3359 | (deoxyribose-5-phosphate <=> Acetaldehyde + | | |
| | | | | Glyceraldehyde3-phosphate), ATP: acetate | | |
| | | | | phosphotransferase (ATP + H+ + Propionate --> ADP + | | |
| | | | | Propionyl phosphate) | | |
| 76 | Yield- | 2 | CAETHG_3021, | L-Arginine iminohydrolase (H2O + L-Arginine <=> NH3 + | 0.21241 | 0.075712 |
| | LMOMA | | CAETHG_3359 | Citrulline), ATP: acetate phosphotransferase (ATP + H+ + | | |
| | | | | Propionate --> ADP + Propionyl phosphate) | | |
| 77 | Yield- | 2 | 0, | Acetyl-CoA: carbon-dioxide ligase (ADP-forming) (ATP + | 0.21241 | 0.075524 |
| | LMOMA | | CAETHG_3359 | Acetyl-CoA + H2CO3 --> ADP + Phosphate + H+ + Malonyl- | | |
| | | | | CoA), ATP: acetate phosphotransferase (ATP + H+ + | | |
| | | | | Propionate --> ADP + Propionyl phosphate) | | |
| 78 | Yield- | 2 | CAETHG_2107, | Potassium uptake (K+_ext <=> K+), ATP: acetate | 0.21241 | 0.075492 |
| | LMOMA | | CAETHG_3359 | phosphotransferase (ATP + H+ + Propionate --> ADP + | | |
| | | | | Propionyl phosphate) | | |
| 79 | Yield- | 2 | CAETHG_2107, | Potassium uptake (K+_ext <=> K+), Phosphate | 0.21241 | 0.075492 |
| | LMOMA | | CAETHG_3358 | transacetylase (Phosphate + Acetyl-CoA + H+ <=> CoA + | | |
| | | | | Acetylphosphate) | | |
| 80 | Yield- | 2 | CAETHG_3164, | UMP: pyrophosphate phosphoribosyltransferase (Uracil + | 0.21241 | 0.075488 |
| | LMOMA | | CAETHG_3359 | PRPP --> PPi + UMP), ATP: acetate phosphotransferase | | |
| | | | | (ATP + H+ + Propionate --> ADP + Propionyl phosphate) | | |
| 81 | Yield- | 2 | CAETHG_3164, | UMP: pyrophosphate phosphoribosyltransferase (Uracil + | 0.21671 | 0.075488 |
| | LMOMA | | CAETHG_3358 | PRPP --> PPi + UMP), Phosphate transacetylase | | |
| | | | | (Phosphate + Acetyl-CoA + H+ <=> CoA + | | |
| | | | | Acetylphosphate) | | |
| 82 | Yield- | 1 | CAETHG_3358 | Phosphate transacetylase (Phosphate + Acetyl-CoA + H+ <=> | 0.21671 | 0.075484 |
| | LMOMA | | | CoA + Acetylphosphate) | | |
| 83 | Yield- | 1 | CAETHG_3359 | ATP: acetate phosphotransferase (ATP + H+ + Propionate--> | 0.21241 | 0.075484 |
| | LMOMA | | | ADP + Propionyl phosphate) | | |
| 84 | Yield- | 2 | CAETHG_1224, | L-serine ammonia-lyase (L-Serine --> NH3 + Pyruvate), N- | 0 | 0.0213 |
| | LMOMA | | CAETHG_0160 | Ribosylnicotinamide: orthophosphate ribosyltransferase | | |
| | | | | (Phosphate + N-Ribosylnicotinamide <=> Nicotinamide + | | |
| | | | | Ribose 1-phosphate) | | |
| 85 | Yield- | 2 | CAETHG_0160, | N-Ribosylnicotinamide: orthophosphate | 0 | 0.021224 |
| | LMOMA | | CAETHG_2107 | ribosyltransferase (Phosphate + N-Ribosylnicotinamide <=> | | |
| | | | | Nicotinamide + Ribose 1-phosphate), Potassium | | |
| | | | | uptake (K+_ext <=> K+) | | |
| 86 | Yield- | 1 | CAETHG_1270 | AMP: pyrophosphate phosphoribosyltransferase (PPi + | 0 | 0.02122 |
| | LMOMA | | | AMP <=> PRPP + Adenine) | | |
| 87 | Yield- | 1 | CAETHG_3924 | D-Ribose 1,5-phosphomutase (Ribose 1-phosphate <=> | 0 | 0.02122 |
| | LMOMA | | | ribose-5-phosphate) | | |
| 88 | Yield- | 1 | CAETHG_1371 | Adenosine 5′-monophosphate phosphohydrolase (H2O + | 0 | 0.02122 |
| | LMOMA | | | AMP <=> Phosphate + H+ + Adenosine) | | |
| 89 | Yield- | 1 | CAETHG_0160 | N-Ribosylnicotinamide: orthophosphate | 0 | 0.02122 |
| | LMOMA | | | ribosyltransferase (Phosphate + N-Ribosylnicotinamide <=> | | |
| | | | | Nicotinamide + Ribose 1-phosphate) | | |
| 90 | Yield- | 2 | CAETHG_0248, | L-lactate reversible transport via proton symport (H+_ext + | 0 | 0.020644 |
| | LMOMA | | CAETHG_2721 | L-Lactate_ext <=> H+ + L-Lactate), | | |
| | | | | Phosphoenolpyruvate carboxykinase (PPCK) (ATP + | | |
| | | | | Oxaloacetate + H+ --> ADP + CO2 + | | |
| | | | | Phosphoenolpyruvate) | | |
| 91 | Yield- | 2 | CAETHG_2107, | Potassium uptake (K+_ext <=> K+), Phosphoenolpyruvate | 0 | 0.020584 |
| | LMOMA | | CAETHG_2721 | carboxykinase (PPCK) (ATP + Oxaloacetate + H+ --> ADP + | | |
| | | | | CO2 + Phosphoenolpyruvate) | | |
| 92 | Yield- | 1 | CAETHG_2721 | Phosphoenolpyruvate carboxykinase (PPCK) (ATP + | 0 | 0.02058 |
| | LMOMA | | | Oxaloacetate + H+ --> ADP + CO2 + | | |
| | | | | Phosphoenolpyruvate) | | |
| 93 | Yield- | 1 | CAETHG_3021 | L-Arginine iminohydrolase (H2O + L-Arginine <=> NH3 + | 0 | 0.02006 |
| | LMOMA | | | Citrulline) | | |
| 94 | BPCY- | 4 | CAETHG_2751, | Citramalate synthase (H2O + Pyruvate + Acetyl-CoA <=> | 0.05233 | 0.012686 |
| | FBA | | CAETHG_2753, | CoA + Citramalate), Isocitrate dehydrogenase (NAD + | | |
| | | | CAETHG_2909, | Isocitrate <=> NADH + CO2 + 2-Oxoglutarate + H+), | | |
| | | | CAETHG_3293 | ATP: pyruvate, orthophosphate phosphotransferase (ATP + | | |
| | | | | Phosphate + Pyruvate + H+ --> PPi + AMP + | | |
| | | | | Phosphoenolpyruvate), Cysteine desulfhydrase (H2O + L- | | |
| | | | | Cysteine <=> NH3 + Pyruvate + H2S) | | |
| 95 | BPCY- | 3 | CAETHG_2753, | Isocitrate dehydrogenase (NAD + Isocitrate <=> NADH + | 0.02686 | 0.00855 |
| | FBA | | CAETHG_2909, | CO2 + 2-Oxoglutarate + H+), | | |
| | | | CAETHG_0498 | ATP: pyruvate, orthophosphate phosphotransferase (ATP + | | |
| | | | | Phosphate + Pyruvate + H+ --> PPi + AMP + | | |
| | | | | Phosphoenolpyruvate), Cystathionine beta lyase (H2O + | | |
| | | | | Cystathionine --> NH3 + Pyruvate + Homocysteine) | | |
| 96 | BPCY- | 3 | CAETHG_2751, | Citramalate synthase (H2O + Pyruvate + Acetyl-CoA <=> | 0.02545 | 0.008366 |
| | FBA | | CAETHG_2753, | CoA + Citramalate), Isocitrate dehydrogenase (NAD + | | |
| | | | CAETHG_2909 | Isocitrate <=> NADH + CO2 + 2-Oxoglutarate + H+), | | |
| | | | | ATP: pyruvate, orthophosphate phosphotransferase (ATP + | | |
| | | | | Phosphate + Pyruvate + H+ --> PPi + AMP + | | |
| | | | | Phosphoenolpyruvate) | | |
| 97 | BPCY- | 2 | CAETHG_2753, | Isocitrate dehydrogenase (NAD + Isocitrate <=> NADH + | 0.01873 | 0.007195 |
| | FBA | | CAETHG_2909 | CO2 + 2-Oxoglutarate + H+), | | |
| | | | | ATP: pyruvate, orthophosphate phosphotransferase (ATP + | | |
| | | | | Phosphate + Pyruvate + H+ --> PPi + AMP + | | |
| | | | | Phosphoenolpyruvate) | | |
| 98 | BPCY- | 3 | CAETHG_2751, | Citramalate synthase (H2O + Pyruvate + Acetyl-CoA <=> | 0 | 0.002528 |
| | FBA | | CAETHG_2909, | CoA + Citramalate), ATP: pyruvate, orthophosphate | | |
| | | | CAETHG_3293 | phosphotransferase (ATP + Phosphate + Pyruvate + H+ --> | | |
| | | | | PPi + AMP + Phosphoenolpyruvate), Cysteine | | |
| | | | | desulfhydrase (H2O + L-Cysteine <=> NH3 + Pyruvate + | | |
| | | | | H2S) | | |
| 99 | BPCY- | 3 | CAETHG_2751, | Citramalate synthase (H2O + Pyruvate + Acetyl-CoA <=> | 0 | 0.002508 |
| | FBA | | CAETHG_2909, | CoA + Citramalate), ATP: pyruvate, orthophosphate | | |
| | | | CAETHG_3611 | phosphotransferase (ATP + Phosphate + Pyruvate + H+ --> | | |
| | | | | PPi + AMP + Phosphoenolpyruvate), Threonine | | |
| | | | | dehydratase (L-Threonine --> NH3 + 2-Oxobutyrate) | | |
| 100 | BPCY- | 2 | CAETHG_2751, | Citramalate synthase (H2O + Pyruvate + Acetyl-CoA <=> | 0 | 0.001842 |
| | FBA | | CAETHG_2909 | CoA + Citramalate), ATP: pyruvate, orthophosphate | | |
| | | | | phosphotransferase (ATP + Phosphate + Pyruvate + H+ --> | | |
| | | | | PPi + AMP + Phosphoenolpyruvate) | | |
| 101 | BPCY- | 2 | CAETHG_2909, | ATP: pyruvate, orthophosphate phosphotransferase (ATP + | 0 | 0.001323 |
| | FBA | | CAETHG_3293 | Phosphate + Pyruvate + H+ --> PPi + AMP + | | |
| | | | | Phosphoenolpyruvate), Cysteine desulfhydrase (H2O + L- | | |
| | | | | Cysteine <=> NH3 + Pyruvate + H2S) | | |
| 102 | BPCY- | 2 | CAETHG_2753, | Isocitrate dehydrogenase (NAD + Isocitrate <=> NADH + | 0 | 0.000573 |
| | FBA | | CAETHG_3293 | CO2 + 2-Oxoglutarate + H+), Cysteine desulfhydrase (H2O + | | |
| | | | | L-Cysteine <=> NH3 + Pyruvate + H2S) | | |
| 103 | BPCY- | 1 | CAETHG_2909 | ATP: pyruvate, orthophosphate phosphotransferase (ATP + | 0 | 0.000573 |
| | FBA | | | Phosphate + Pyruvate + H+ --> PPi + AMP + | | |
| | | | | Phosphoenolpyruvate) | | |

### Example 11

This example describes disruptions for improved production of 3-hydroxybutyrate in Wood-Ljungdahl microorganisms. Metabolic modeling experiments were performed as described in Example 1. Production of 3-hydroxybutyrate in Wood-Ljungdahl microorganisms is described, e.g., in WO 2017/066498. The following pathway was used to model 3-hydroxybutyrate production herein: 3-Hydroxybutyrate-->3-Hydroxybutyrate_ext; Acetate+Acetoacetyl-CoA-->Acetyl-CoA+Acetoacetate; NADH+H++Acetoacetate-->NAD+3-Hydroxybutyrate; 2.0 Acetyl-CoA-->CoA+Acetoacetyl-CoA.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | BPCY- | 3 | CAETHG_2753, | Isocitrate dehydrogenase (NAD + Isocitrate <=> NADH + | 0.01233 | 0.00434 |
| | FBA | | CAETHG_2909, | CO2 + 2-Oxoglutarate + H+), ATP: pyruvate, orthophosphate | | |
| | | | CAETHG_3293 | phosphotransferase (ATP + Phosphate + Pyruvate + H+ --> | | |
| | | | | PPi + AMP + Phosphoenolpyruvate), Cysteine desulfhydrase | | |
| | | | | (H2O + L-Cysteine <=> NH3 + Pyruvate + H2S) | | |
| 2 | BPCY- | 3 | CAETHG_2753, | Isocitrate dehydrogenase (NAD + Isocitrate <=> NADH + | 0 | 0.002583 |
| | FBA | | CAETHG_2909, | CO2 + 2-Oxoglutarate + H+), ATP: pyruvate, orthophosphate | | |
| | | | CAETHG_0498 | phosphotransferase (ATP + Phosphate + Pyruvate + H+ --> | | |
| | | | | PPi + AMP + Phosphoenolpyruvate), Cystathionine beta | | |
| | | | | lyase (H2O + Cystathionine --> NH3 + Pyruvate + | | |
| | | | | Homocysteine) | | |
| 3 | BPCY- | 3 | CAETHG_2751, | Citramalate synthase (H2O + Pyruvate + Acetyl-CoA <=> | 0 | 0.002477 |
| | FBA | | CAETHG_2753, | CoA + Citramalate), Isocitrate dehydrogenase (NAD + | | |
| | | | CAETHG_2909 | Isocitrate <=> NADH + CO2 + 2-Oxoglutarate + H+), | | |
| | | | | ATP: pyruvate, orthophosphate phosphotransferase (ATP + | | |
| | | | | Phosphate + Pyruvate + H+ --> PPi + AMP + | | |
| | | | | Phosphoenolpyruvate) | | |
| 4 | BPCY- | 2 | CAETHG_2753, | Isocitrate dehydrogenase (NAD + Isocitrate <=> NADH + | 0 | 0.001788 |
| | FBA | | CAETHG_2909 | CO2 + 2-Oxoglutarate + H+), ATP: pyruvate, orthophosphate | | |
| | | | | phosphotransferase (ATP + Phosphate + Pyruvate + H+ --> | | |
| | | | | PPi + AMP + Phosphoenolpyruvate) | | |
| 5 | BPCY- | 2 | CAETHG_2753, | Isocitrate dehydrogenase (NAD + Isocitrate <=> NADH + | 0 | 0.001475 |
| | FBA | | CAETHG_2909 | CO2 + 2-Oxoglutarate + H+), ATP: pyruvate, orthophosphate | | |
| | | | | phosphotransferase (ATP + Phosphate + Pyruvate + H+ --> | | |
| | | | | PPi + AMP + Phosphoenolpyruvate) | | |
| 6 | Yield- | 6 | CAETHG_0160, | N-Ribosylnicotinamide: orthophosphate ribosyltransferase | 0 | 0.090072 |
| | LMOMA | | CAETHG_2721, | (Phosphate + N-Ribosylnicotinamide <=> Nicotinamide + | | |
| | | | CAETHG_2932, | Ribose 1-phosphate), Phosphoenolpyruvate carboxykinase | | |
| | | | CAETHG_3359, | (PPCK) (ATP + Oxaloacetate + H+ --> ADP + CO2 + | | |
| | | | CAETHG_0475, | Phosphoenolpyruvate), Alpha-acetolactate decarboxylase | | |
| | | | CAETHG_0498 | (ALCTT --> CO2 + (R)-Acetoin), ATP: acetate | | |
| | | | | phosphotransferase (ATP + H+ + Propionate --> ADP + | | |
| | | | | Propionyl phosphate), Cystathionine beta lyase (H2O + | | |
| | | | | Cystathionine --> NH3 + Pyruvate + Homocysteine) | | |
| 7 | Yield- | 5 | CAETHG_0160, | N-Ribosylnicotinamide: orthophosphate ribosyltransferase | 0 | 0.088912 |
| | LMOMA | | CAETHG_2721, | (Phosphate + N-Ribosylnicotinamide <=> Nicotinamide + | | |
| | | | CAETHG_2932, | Ribose 1-phosphate), Phosphoenolpyruvate carboxykinase | | |
| | | | CAETHG_3359, | (PPCK) (ATP + Oxaloacetate + H+ --> ADP + CO2 + | | |
| | | | CAETHG_0475 | Phosphoenolpyruvate), Alpha-acetolactate decarboxylase | | |
| | | | | (ALCTT --> CO2 + (R)-Acetoin), ATP: acetate | | |
| | | | | phosphotransferase (ATP + H+ + Propionate --> ADP + | | |
| | | | | Propionyl phosphate) | | |
| 8 | Yield- | 5 | CAETHG_0160, | N-Ribosylnicotinamide: orthophosphate ribosyltransferase | 0 | 0.088912 |
| | LMOMA | | CAETHG_2721, | (Phosphate + N-Ribosylnicotinamide <=> Nicotinamide + | | |
| | | | CAETHG_2932, | Ribose 1-phosphate), Phosphoenolpyruvate carboxykinase | | |
| | | | CAETHG_3358, | (PPCK) (ATP + Oxaloacetate + H+ --> ADP + CO2 + | | |
| | | | CAETHG_0475 | Phosphoenolpyruvate), Alpha-acetolactate decarboxylase | | |
| | | | | (ALCTT --> CO2 + (R)-Acetoin), Phosphate transacetylase | | |
| | | | | (Phosphate + Acetyl-CoA + H+ <=> CoA + Acetylphosphate) | | |
| 9 | Yield- | 4 | CAETHG_2721, | Phosphoenolpyruvate carboxykinase (PPCK) (ATP + | 0 | 0.08852 |
| | LMOMA | | CAETHG_2932, | Oxaloacetate + H+ --> ADP + CO2 + Phosphoenolpyruvate), | | |
| | | | CAETHG_3359, | Alpha-acetolactate decarboxylase (ALCTT --> CO2 + (R)- | | |
| | | | CAETHG_3510 | Acetoin), ATP: acetate phosphotransferase (ATP + H+ + | | |
| | | | | Propionate --> ADP + Propionyl phosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 10 | Yield- | 4 | CAETHG_2721, | Phosphoenolpyruvate carboxykinase (PPCK) (ATP + | 0 | 0.0884 |
| | LMOMA | | CAETHG_2932, | Oxaloacetate + H+ --> ADP + CO2 + Phosphoenolpyruvate), | | |
| | | | CAETHG_3359, | Alpha-acetolactate decarboxylase (ALCTT --> CO2 + (R)- | | |
| | | | CAETHG_0474 | Acetoin), ATP: acetate phosphotransferase (ATP + H+ + | | |
| | | | | Propionate --> ADP + Propionyl phosphate), | | |
| | | | | Glycinedipoylprotein oxidoreductase (decarboxylating and | | |
| | | | | acceptor-aminomethylating) (Glycine + H+ + Lipoylprotein --> | | |
| | | | | CO2 + S-Aminomethyldihydrolipoylprotein) | | |
| 11 | Yield- | 4 | CAETHG_2721, | Phosphoenolpyruvate carboxykinase (PPCK) (ATP + | 0 | 0.0884 |
| | LMOMA | | CAETHG_2932, | Oxaloacetate + H+ --> ADP + CO2 + Phosphoenolpyruvate), | | |
| | | | CAETHG_3359, | Alpha-acetolactate decarboxylase (ALCTT --> CO2 + (R)- | | |
| | | | CAETHG_0475 | Acetoin), ATP: acetate phosphotransferase (ATP + H+ + | | |
| | | | | Propionate --> ADP + Propionyl phosphate) | | |
| 12 | Yield- | 3 | CAETHG_3359, | ATP: acetate phosphotransferase (ATP + H+ + Propionate --> | 0 | 0.087852 |
| | LMOMA | | CAETHG_3510, | ADP + Propionyl phosphate), 2,6-Diaminoheptanedioate: 2- | | |
| | | | CAETHG_0498 | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 13 | Yield- | 3 | CAETHG_3021, | L-Arginine iminohydrolase (H2O + L-Arginine <=> NH3 + | 0 | 0.087504 |
| | LMOMA | | CAETHG_3359, | Citrulline), ATP: acetate phosphotransferase (ATP + H+ + | | |
| | | | CAETHG_3510 | Propionate --> ADP + Propionyl phosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 14 | Yield- | 2 | CAETHG_3359, | ATP: acetate phosphotransferase (ATP + H+ + Propionate --> | 0 | 0.087464 |
| | LMOMA | | CAETHG_3510 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 15 | Yield- | 2 | CAETHG_2721, | Phosphoenolpyruvate carboxykinase (PPCK) (ATP + | 0 | 0.079284 |
| | LMOMA | | CAETHG_3359 | Oxaloacetate + H+ --> ADP + CO2 + Phosphoenolpyruvate), | | |
| | | | | ATP: acetate phosphotransferase (ATP + H+ + Propionate --> | | |
| | | | | ADP + Propionyl phosphate) | | |
| 16 | Yield- | 2 | CAETHG_2721, | Phosphoenolpyruvate carboxykinase (PPCK) (ATP + | 0 | 0.079284 |
| | LMOMA | | CAETHG_3358 | Oxaloacetate + H+ --> ADP + CO2 + Phosphoenolpyruvate), | | |
| | | | | Phosphate transacetylase (Phosphate + Acetyl-CoA +H+ <=> | | |
| | | | | CoA + Acetylphosphate) | | |
| 17 | Yield- | 2 | CAETHG_2932, | Alpha-acetolactate decarboxylase (ALCTT --> CO2 + (R)- | 0 | 0.073692 |
| | LMOMA | | CAETHG_3358 | Acetoin), Phosphate transacetylase (Phosphate + Acetyl- | | |
| | | | | CoA + H+ <=> CoA + Acetylphosphate) | | |
| 18 | Yield- | 2 | CAETHG_3358, | Phosphate transacetylase (Phosphate + Acetyl-CoA + H+ <=> | 0 | 0.057428 |
| | LMOMA | | CAETHG_0686 | CoA + Acetylphosphate), L-Threonine acetaldehyde- | | |
| | | | | lyase (L-Threonine --> Glycine + Acetaldehyde) | | |
| 19 | Yield- | 2 | CAETHG_3358, | Phosphate transacetylase (Phosphate + Acetyl-CoA + H+ <=> | 0 | 0.054844 |
| | LMOMA | | CAETHG_0498 | CoA + Acetylphosphate), Cystathionine beta lyase (H2O + | | |
| | | | | Cystathionine --> NH3 + Pyruvate + Homocysteine) | | |
| 20 | Yield- | 2 | CAETHG_1371, | Adenosine 5′-monophosphate phosphohydrolase (H2O + | 0 | 0.054664 |
| | LMOMA | | CAETHG_3358 | AMP <=> Phosphate + H+ + Adenosine), Phosphate | | |
| | | | | transacetylase (Phosphate + Acetyl-CoA + H+ <=> CoA + | | |
| | | | | Acetylphosphate) | | |
| 21 | Yield- | 2 | CAETHG_3358, | Phosphate transacetylase (Phosphate + Acetyl-CoA + H+ <=> | 0 | 0.054664 |
| | LMOMA | | CAETHG_3924 | CoA + Acetylphosphate), D-Ribose 1,5-phosphomutase | | |
| | | | | (Ribose 1-phosphate <=> ribose-5-phosphate) | | |
| 22 | Yield- | 2 | CAETHG_0160, | N-Ribosylnicotinamide: orthophosphate ribosyltransferase | 0 | 0.054664 |
| | LMOMA | | CAETHG_3359 | (Phosphate + N-Ribosylnicotinamide <=> Nicotinamide + | | |
| | | | | Ribose 1-phosphate), ATP: acetate phosphotransferase (ATP + | | |
| | | | | H+ + Propionate --> ADP + Propionyl phosphate) | | |
| 23 | Yield- | 2 | CAETHG_0160, | N-Ribosylnicotinamide: orthophosphate ribosyltransferase | 0 | 0.054664 |
| | LMOMA | | CAETHG_3358 | (Phosphate + N-Ribosylnicotinamide <=> Nicotinamide + | | |
| | | | | Ribose 1-phosphate), Phosphate transacetylase (Phosphate + | | |
| | | | | Acetyl-CoA + H+ <=> CoA + Acetylphosphate) | | |
| 24 | Yield- | 2 | CAETHG_1270, | AMP: pyrophosphate phosphoribosyltransferase (PPi + AMP <=> | 0 | 0.05458 |
| | LMOMA | | CAETHG_3358 | PRPP + Adenine), Phosphate transacetylase (Phosphate + | | |
| | | | | Acetyl-CoA + H+ <=> CoA + Acetylphosphate) | | |
| 25 | Yield- | 2 | CAETHG_2475, | dGTP triphosphohydrolase (H2O + dGTP --> H+ + | 0 | 0.053004 |
| | LMOMA | | CAETHG_3358 | Deoxyguanosine + Triphosphate), Phosphate transacetylase | | |
| | | | | (Phosphate + Acetyl-CoA + H+ <=> CoA + Acetylphosphate) | | |
| 26 | Yield- | 2 | CAETHG_2799, | NADP-dependent electron-bifurcating [FeFe]-hydrogenase | 0 | 0.052724 |
| | LMOMA | | CAETHG_3358 | (Hyt) (NADP + Oxidizedferredoxin + 2.0 H2 <=> NADPH + 3.0 | | |
| | | | | H+ + Reducedferredoxin), Phosphate transacetylase | | |
| | | | | (Phosphate + Acetyl-CoA + H+ <=> CoA + Acetylphosphate) | | |
| 27 | Yield- | 2 | CAETHG_2789, | Formate hydrogen-lyase (also known as hydrogen- | 0 | 0.052724 |
| | LMOMA | | CAETHG_3358 | dependent CO2 reductase) (CO2 + H2 --> Formate + H+), | | |
| | | | | Phosphate transacetylase (Phosphate + Acetyl-CoA + H+ <=> | | |
| | | | | CoA + Acetylphosphate) | | |
| 28 | Yield- | 2 | CAETHG_2795, | NADP-dependent electron-bifurcating [FeFe]-hydrogenase | 0 | 0.052724 |
| | LMOMA | | CAETHG_3358 | (Hyt) (NADP + Oxidizedferredoxin + 2.0 H2 <=> NADPH + 3.0 | | |
| | | | | H+ + Reducedferredoxin), Phosphate transacetylase | | |
| | | | | (Phosphate + Acetyl-CoA + H+ <=> CoA + Acetylphosphate) | | |
| 29 | Yield- | 2 | CAETHG_2794, | NADP-dependent electron-bifurcating [FeFe]-hydrogenase | 0 | 0.052724 |
| | LMOMA | | CAETHG_3358 | (Hyt) (NADP + Oxidizedferredoxin + 2.0 H2 <=> NADPH + 3.0 | | |
| | | | | H+ + Reducedferredoxin), Phosphate transacetylase | | |
| | | | | (Phosphate + Acetyl-CoA + H+ <=> CoA + Acetylphosphate) | | |
| 30 | Yield- | 2 | CAETHG_2796, | NADP-dependent electron-bifurcating [FeFe]-hydrogenase | 0 | 0.052724 |
| | LMOMA | | CAETHG_3358 | (Hyt) (NADP + Oxidizedferredoxin + 2.0 H2 <=> NADPH + 3.0 | | |
| | | | | H+ + Reducedferredoxin), Phosphate transacetylase | | |
| | | | | (Phosphate + Acetyl-CoA + H+ <=> CoA + Acetylphosphate) | | |
| 31 | Yield- | 2 | CAETHG_2791, | Formate hydrogen-lyase (also known as hydrogen- | 0 | 0.052724 |
| | LMOMA | | CAETHG_3358 | dependent CO2 reductase) (CO2 + H2 --> Formate + H+), | | |
| | | | | Phosphate transacetylase (Phosphate + Acetyl-CoA + H+ <=> | | |
| | | | | CoA + Acetylphosphate) | | |
| 32 | Yield- | 2 | CAETHG_2790, | Formate hydrogen-lyase (also known as hydrogen- | 0 | 0.052724 |
| | LMOMA | | CAETHG_3358 | dependent CO2 reductase) (CO2 + H2 --> Formate + H+), | | |
| | | | | Phosphate transacetylase (Phosphate + Acetyl-CoA + H+ <=> | | |
| | | | | CoA + Acetylphosphate) | | |
| 33 | Yield- | 2 | CAETHG_2798, | NADP-dependent electron-bifurcating [FeFe]-hydrogenase | 0 | 0.052724 |
| | LMOMA | | CAETHG_3358 | (Hyt) (NADP + Oxidizedferredoxin + 2.0 H2 <=> NADPH + 3.0 | | |
| | | | | H+ + Reducedferredoxin), Phosphate transacetylase | | |
| | | | | (Phosphate + Acetyl-CoA + H+ <=> CoA + Acetylphosphate) | | |
| 34 | Yield- | 2 | CAETHG_2793, | Formate hydrogen-lyase (also known as hydrogen- | 0 | 0.052724 |
| | LMOMA | | CAETHG_3358 | dependent CO2 reductase) (CO2 + H2 --> Formate + H+), | | |
| | | | | Phosphate transacetylase (Phosphate + Acetyl-CoA + H+ <=> | | |
| | | | | CoA + Acetylphosphate) | | |
| 35 | Yield- | 2 | CAETHG_3293, | Cysteine desulfhydrase (H2O + L-Cysteine <=> NH3 + | 0 | 0.052712 |
| | LMOMA | | CAETHG_3358 | Pyruvate + H2S), Phosphate transacetylase (Phosphate + | | |
| | | | | Acetyl-CoA + H+ <=> CoA + Acetylphosphate) | | |
| 36 | Yield- | 2 | CAETHG_3021, | L-Arginine iminohydrolase (H2O + L-Arginine <=> NH3 + | 0 | 0.052652 |
| | LMOMA | | CAETHG_3358 | Citrulline), Phosphate transacetylase (Phosphate + Acetyl- | | |
| | | | | CoA + H+ <=> CoA + Acetylphosphate) | | |
| 37 | Yield- | 2 | 0, | Acetyl-CoA: carbon-dioxide ligase (ADP-forming) (ATP + | 0 | 0.052616 |
| | LMOMA | | CAETHG_3358 | Acetyl-CoA + H2CO3 --> ADP + Phosphate + H+ + Malonyl- | | |
| | | | | CoA), Phosphate transacetylase (Phosphate + Acetyl-CoA + | | |
| | | | | H+ <=> CoA + Acetylphosphate) | | |
| 38 | Yield- | 2 | CAETHG_0233, | 4-imidazolone-5-propanoate amidohydrolase (H2O + 4- | 0 | 0.052616 |
| | LMOMA | | CAETHG_3358 | Imidazolone-5-propanoate > N Formimino-L-glutamate), | | |
| | | | | Phosphate transacetylase (Phosphate + Acetyl-CoA + H+ <=> | | |
| | | | | CoA + Acetylphosphate) | | |
| 39 | Yield- | 2 | CAETHG_0234, | 4,5-Dihydro-4-oxo-5-imidazolepropanoate hydro-lyase (4- | 0 | 0.052616 |
| | LMOMA | | CAETHG_3358 | Imidazolone-5-propanoate <=> H2O + Urocanate), | | |
| | | | | Phosphate transacetylase (Phosphate + Acetyl-CoA + H+ <=> | | |
| | | | | CoA + Acetylphosphate) | | |
| 40 | Yield- | 2 | CAETHG_0234, | 4,5-Dihydro-4-oxo-5-imidazolepropanoate hydro-lyase (4- | 0 | 0.052616 |
| | LMOMA | | CAETHG_3359 | Imidazolone-5-propanoate <=> H2O + Urocanate), | | |
| | | | | ATP: acetate phosphotransferase (ATP + H+ + Propionate --> | | |
| | | | | ADP + Propionyl phosphate) | | |
| 41 | Yield- | 2 | CAETHG_3164, | UMP: pyrophosphate phosphoribosyltransferase (Uracil + | 0 | 0.052584 |
| | LMOMA | | CAETHG_3358 | PRPP --> PPi + UMP), Phosphate transacetylase (Phosphate + | | |
| | | | | Acetyl-CoA + H+ <=> CoA + Acetylphosphate) | | |
| 42 | Yield- | 2 | CAETHG_2107, | Potassium uptake (K+_ext <=> K+), Phosphate | 0 | 0.05258 |
| | LMOMA | | CAETHG_3358 | transacetylase (Phosphate + Acetyl-CoA + H+ <=> CoA + | | |
| | | | | Acetylphosphate) | | |
| 43 | Yield- | 1 | CAETHG_3359 | ATP: acetate phosphotransferase (ATP + H+ + Propionate --> | 0 | 0.052576 |
| | LMOMA | | | ADP + Propionyl phosphate) | | |
| 44 | Yield- | 1 | CAETHG_3358 | Phosphate transacetylase (Phosphate + Acetyl-CoA + H+ <=> | 0 | 0.052576 |
| | LMOMA | | | CoA + Acetylphosphate) | | |
| 45 | Yield- | 2 | CAETHG_2721, | Phosphoenolpyruvate carboxykinase (PPCK) (ATP + | 0 | 0.02074 |
| | LMOMA | | CAETHG_2751 | Oxaloacetate + H+ --> ADP + CO2 + Phosphoenolpyruvate), | | |
| | | | | Citramalate synthase (H2O + Pyruvate + Acetyl-CoA <=> | | |
| | | | | CoA + Citramalate) | | |
| 46 | Yield- | 2 | CAETHG_2721, | Phosphoenolpyruvate carboxykinase (PPCK) (ATP + | 0 | 0.0207 |
| | LMOMA | | CAETHG_0498 | Oxaloacetate + H+ --> ADP + CO2 + Phosphoenolpyruvate), | | |
| | | | | Cystathionine beta lyase (H2O + Cystathionine --> NH3 + | | |
| | | | | Pyruvate + Homocysteine) | | |
| 47 | Yield- | 2 | CAETHG_1270, | AMP: pyrophosphate phosphoribosyltransferase (PPi + AMP <=> | 0 | 0.020628 |
| | LMOMA | | CAETHG_2721 | PRPP + Adenine), Phosphoenolpyruvate carboxykinase | | |
| | | | | (PPCK) (ATP + Oxaloacetate + H+ --> ADP + CO2 + | | |
| | | | | Phosphoenolpyruvate) | | |
| 48 | Yield- | 2 | CAETHG_2721, | Phosphoenolpyruvate carboxykinase (PPCK) (ATP + | 0 | 0.020628 |
| | LMOMA | | CAETHG_3924 | Oxaloacetate + H+ --> ADP + CO2 + Phosphoenolpyruvate), | | |
| | | | | D-Ribose 1,5-phosphomutase (Ribose 1-phosphate <=> | | |
| | | | | ribose-5-phosphate) | | |
| 49 | Yield- | 2 | CAETHG_1224, | L-serine ammonia-lyase (L-Serine --> NH3 + Pyruvate), | 0 | 0.02054 |
| | LMOMA | | CAETHG_2721 | Phosphoenolpyruvate carboxykinase (PPCK) (ATP + | | |
| | | | | Oxaloacetate + H+ --> ADP + CO2 + Phosphoenolpyruvate) | | |
| 50 | Yield- | 2 | CAETHG_1225, | L-serine ammonia-lyase (L-Serine --> NH3 + Pyruvate), | 0 | 0.02054 |
| | LMOMA | | CAETHG_2721 | Phosphoenolpyruvate carboxykinase (PPCK) (ATP + | | |
| | | | | Oxaloacetate + H+ --> ADP + CO2 + Phosphoenolpyruvate) | | |
| 51 | Yield- | 2 | CAETHG_2107, | Potassium uptake (K+_ext <=> K+), Phosphoenolpyruvate | 0 | 0.020448 |
| | LMOMA | | CAETHG_2721 | carboxykinase (PPCK) (ATP + Oxaloacetate + H+ --> ADP + | | |
| | | | | CO2 + Phosphoenolpyruvate) | | |
| 52 | Yield- | 1 | CAETHG_2721 | Phosphoenolpyruvate carboxykinase (PPCK) (ATP + | 0 | 0.020444 |
| | LMOMA | | | Oxaloacetate + H+ --> ADP + CO2 + Phosphoenolpyruvate) | | |
| 53 | Yield- | 1 | CAETHG_1270 | AMP: pyrophosphate phosphoribosyltransferase (PPi + AMP <=> | 0 | 0.020432 |
| | LMOMA | | | PRPP + Adenine) | | |
| 54 | Yield- | 1 | CAETHG_0160 | N-Ribosylnicotinamide: orthophosphate ribosyltransferase | 0 | 0.020432 |
| | LMOMA | | | (Phosphate + N-Ribosylnicotinamide <=> Nicotinamide + | | |
| | | | | Ribose 1-phosphate) | | |
| 55 | Yield- | 1 | CAETHG_1371 | Adenosine 5′-monophosphate phosphohydrolase (H2O + | 0 | 0.020432 |
| | LMOMA | | | AMP <=> Phosphate + H+ + Adenosine) | | |
| 56 | Yield- | 1 | CAETHG_3924 | D-Ribose 1,5-phosphomutase (Ribose 1-phosphate <=> | 0 | 0.020432 |
| | LMOMA | | | ribose-5-phosphate) | | |
| 57 | Yield- | 2 | CAETHG_0234, | 4,5-Dihydro-4-oxo-5-imidazolepropanoate hydro-lyase (4- | 0 | 0.020032 |
| | LMOMA | | CAETHG_3021 | Imidazolone-5-propanoate <=> H2O + Urocanate), L- | | |
| | | | | Arginine iminohydrolase (H2O + L-Arginine <=> NH3 + | | |
| | | | | Citrulline) | | |

### Example 12

This example describes disruptions for improved production of methyl ethyl ketone (MEK), i.e., 2-butanone, in Wood-Ljungdahl microorganisms. Metabolic modeling experiments were performed as described in Example 1. Production of MEK in Wood-Ljungdahl microorganisms is described, e.g., in WO 2012/024522 and WO 2013/185123. The following pathway was used to model methyl ethyl ketone production herein: NADH+H++(R)-Acetoin-->NAD+meso-2,3-Butanediol; meso-2,3-Butanediol-->H₂O+MEK; H++MEK-->H+_ext+MEK_ext.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | Yield- | 5 | 0, | Acetyl-CoA: carbon-dioxide ligase (ADP-forming) (ATP + | 0.04120 | 0.028524 |
| | LMOMA | | CAETHG_0160, | Acetyl-CoA + H2CO3 --> ADP + Phosphate + H+ + Malonyl- | | |
| | | | CAETHG_3359, | CoA), N-Ribosylnicotinamide: orthophosphate | | |
| | | | CAETHG_3510, | ribosyltransferase (Phosphate + N-Ribosylnicotinamide <=> | | |
| | | | CAETHG_0498 | Nicotinamide + Ribose 1-phosphate), ATP: acetate | | |
| | | | | phosphotransferase (ATP + H+ + Propionate --> ADP + | | |
| | | | | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 2 | Yield- | 5 | 0, | Acetyl-CoA: carbon-dioxide ligase (ADP-forming) (ATP + | 0.04120 | 0.028524 |
| | LMOMA | | CAETHG_1371, | Acetyl-CoA + H2CO3 --> ADP + Phosphate + H+ + Malonyl- | | |
| | | | CAETHG_3359, | CoA), Adenosine 5′-monophosphate phosphohydrolase (H2O + | | |
| | | | CAETHG_3510, | AMP <=> Phosphate + H+ + Adenosine), ATP: acetate | | |
| | | | CAETHG_0498 | phosphotransferase (ATP + H+ + Propionate --> ADP + | | |
| | | | | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 3 | Yield- | 5 | 0, | Acetyl-CoA: carbon-dioxide ligase (ADP-forming) (ATP + | 0.04120 | 0.028524 |
| | LMOMA | | CAETHG_3359, | Acetyl-CoA + H2CO3 --> ADP + Phosphate + H+ + Malonyl- | | |
| | | | CAETHG_3510, | CoA), ATP: acetate phosphotransferase (ATP + H+ + | | |
| | | | CAETHG_3924, | Propionate --> ADP + Propionyl phosphate), 2,6- | | |
| | | | CAETHG_0498 | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate), D-Ribose 1,5- | | |
| | | | | phosphomutase (Ribose 1-phosphate <=> ribose-5- | | |
| | | | | phosphate), Cystathionine beta lyase (H2O + Cystathionine --> | | |
| | | | | NH3 + Pyruvate + Homocysteine) | | |
| 4 | Yield- | 5 | 0, | Acetyl-CoA: carbon-dioxide ligase (ADP-forming) (ATP + | 0.04120 | 0.028524 |
| | LMOMA | | CAETHG_1270, | Acetyl-CoA + H2CO3 --> ADP + Phosphate + H+ + Malonyl- | | |
| | | | CAETHG_3359, | CoA), AMP: pyrophosphate phosphoribosyltransferase (PPi + | | |
| | | | CAETHG_3510, | AMP <=> PRPP + Adenine), ATP: acetate phosphotransferase | | |
| | | | CAETHG_0498 | (ATP + H+ + Propionate --> ADP + Propionyl phosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate), Cystathionine beta | | |
| | | | | lyase (H2O + Cystathionine --> NH3 + Pyruvate + | | |
| | | | | Homocysteine) | | |
| 5 | Yield- | 5 | 0, | Acetyl-CoA: carbon-dioxide ligase (ADP-forming) (ATP + | 0.04120 | 0.028508 |
| | LMOMA | | CAETHG_0160, | Acetyl-CoA + H2CO3 --> ADP + Phosphate + H+ + Malonyl- | | |
| | | | CAETHG_3359, | CoA), N-Ribosylnicotinamide: orthophosphate | | |
| | | | CAETHG_3510, | ribosyltransferase (Phosphate + N-Ribosylnicotinamide <=> | | |
| | | | CAETHG_0498 | Nicotinamide + Ribose 1-phosphate), ATP: acetate | | |
| | | | | phosphotransferase (ATP + H+ + Propionate --> ADP + | | |
| | | | | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 6 | Yield- | 5 | 0, | Acetyl-CoA: carbon-dioxide ligase (ADP-forming) (ATP + | 0.04120 | 0.028476 |
| | LMOMA | | CAETHG_0160, | Acetyl-CoA + H2CO3 --> ADP + Phosphate + H+ + Malonyl- | | |
| | | | CAETHG_3021, | CoA), N-Ribosylnicotinamide: orthophosphate | | |
| | | | CAETHG_3359, | ribosyltransferase (Phosphate + N-Ribosylnicotinamide <=> | | |
| | | | CAETHG_3510 | Nicotinamide + Ribose 1-phosphate), L-Arginine | | |
| | | | | iminohydrolase (H2O + L-Arginine <=> NH3 + Citrulline), | | |
| | | | | ATP: acetate phosphotransferase (ATP + H+ + Propionate --> | | |
| | | | | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 7 | Yield- | 5 | CAETHG_0160, | N-Ribosylnicotinamide: orthophosphate ribosyltransferase | 0.04287 | 0.028464 |
| | LMOMA | | CAETHG_2751, | (Phosphate + N-Ribosylnicotinamide <=> Nicotinamide + | | |
| | | | CAETHG_3359, | Ribose 1-phosphate), Citramalate synthase (H2O + Pyruvate + | | |
| | | | CAETHG_3510, | Acetyl-CoA <=> CoA + Citramalate), ATP: acetate | | |
| | | | CAETHG_0498 | phosphotransferase (ATP + H+ + Propionate --> ADP + | | |
| | | | | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 8 | Yield- | 4 | 0, | Acetyl-CoA: carbon-dioxide ligase (ADP-forming) (ATP + | 0.04120 | 0.02844 |
| | LMOMA | | CAETHG_0160, | Acetyl-CoA + H2CO3 --> ADP + Phosphate + H+ + Malonyl- | | |
| | | | CAETHG_3359, | CoA), N-Ribosylnicotinamide: orthophosphate | | |
| | | | CAETHG_3510 | ribosyltransferase (Phosphate + N-Ribosylnicotinamide <=> | | |
| | | | | Nicotinamide + Ribose 1-phosphate), ATP: acetate | | |
| | | | | phosphotransferase (ATP + H+ + Propionate --> ADP + | | |
| | | | | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 9 | Yield- | 4 | 0, | Acetyl-CoA: carbon-dioxide ligase (ADP-forming) (ATP + | 0.04120 | 0.02844 |
| | LMOMA | | CAETHG_3359, | Acetyl-CoA + H2CO3 --> ADP + Phosphate + H+ + Malonyl- | | |
| | | | CAETHG_3510, | CoA), ATP: acetate phosphotransferase (ATP + H+ + | | |
| | | | CAETHG_3924 | Propionate --> ADP + Propionyl phosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate), D-Ribose 1,5- | | |
| | | | | phosphomutase (Ribose 1-phosphate <=> ribose-5- | | |
| | | | | phosphate) | | |
| 10 | Yield- | 4 | 0, | Acetyl-CoA: carbon-dioxide ligase (ADP-forming) (ATP + | 0.04120 | 0.02844 |
| | LMOMA | | CAETHG_1270, | Acetyl-CoA + H2CO3 --> ADP + Phosphate + H+ + Malonyl- | | |
| | | | CAETHG_3359, | CoA), AMP: pyrophosphate phosphoribosyltransferase (PPi + | | |
| | | | CAETHG_3510 | AMP <=> PRPP + Adenine), ATP: acetate phosphotransferase | | |
| | | | | (ATP + H+ + Propionate --> ADP + Propionyl phosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 11 | Yield- | 4 | CAETHG_3021, | L-Arginine iminohydrolase (H2O + L-Arginine <=> NH3 + | 0.04120 | 0.028296 |
| | LMOMA | | CAETHG_3359, | Citrulline), ATP: acetate phosphotransferase (ATP + H+ + | | |
| | | | CAETHG_3510, | Propionate --> ADP + Propionyl phosphate), 2,6- | | |
| | | | CAETHG_0498 | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate), Cystathionine beta | | |
| | | | | lyase (H2O + Cystathionine --> NH3 + Pyruvate + | | |
| | | | | Homocysteine) | | |
| 12 | Yield- | 3 | CAETHG_3359, | ATP: acetate phosphotransferase (ATP + H+ + Propionate --> | 0.04120 | 0.02828 |
| | LMOMA | | CAETHG_3510, | ADP + Propionyl phosphate), 2,6-Diaminoheptanedioate: 2- | | |
| | | | CAETHG_0498 | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 13 | Yield- | 3 | CAETHG_3021, | L-Arginine iminohydrolase (H2O + L-Arginine <=> NH3 + | 0.04120 | 0.028256 |
| | LMOMA | | CAETHG_3359, | Citrulline), ATP: acetate phosphotransferase (ATP + H+ + | | |
| | | | CAETHG_3510 | Propionate --> ADP + Propionyl phosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 14 | Yield- | 3 | CAETHG_3359, | ATP: acetate phosphotransferase (ATP + H+ + Propionate --> | 0.04120 | 0.028252 |
| | LMOMA | | CAETHG_3510, | ADP + Propionyl phosphate), 2,6-Diaminoheptanedioate: 2- | | |
| | | | CAETHG_0498 | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 15 | Yield- | 3 | CAETHG_3293, | Cysteine desulfhydrase (H2O + L-Cysteine <=> NH3 + | 0.04120 | 0.028248 |
| | LMOMA | | CAETHG_3359, | Pyruvate + H25), ATP: acetate phosphotransferase (ATP + H+ + | | |
| | | | CAETHG_3510 | Propionate --> ADP + Propionyl phosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 16 | Yield- | 2 | CAETHG_3359, | ATP: acetate phosphotransferase (ATP + H+ + Propionate --> | 0.04120 | 0.02824 |
| | LMOMA | | CAETHG_3510 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 17 | Yield- | 2 | CAETHG_3358, | Phosphate transacetylase (Phosphate + Acetyl-CoA + H+ <=> | 0 | 0.02824 |
| | LMOMA | | CAETHG_3510 | CoA + Acetylphosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 18 | Yield- | 3 | CAETHG_3021, | L-Arginine iminohydrolase (H2O + L-Arginine <=> NH3 + | 0 | 0.028224 |
| | LMOMA | | CAETHG_3359, | Citrulline), ATP: acetate phosphotransferase (ATP + H+ + | | |
| | | | CAETHG_3510 | Propionate --> ADP + Propionyl phosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 19 | Yield- | 2 | CAETHG_3359, | ATP: acetate phosphotransferase (ATP + H+ + Propionate --> | 0 | 0.028216 |
| | LMOMA | | CAETHG_3510 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 20 | Yield- | 3 | CAETHG_0160, | N-Ribosylnicotinamide: orthophosphate ribosyltransferase | 0 | 0.02774 |
| | LMOMA | | CAETHG_3359, | (Phosphate + N-Ribosylnicotinamide <=> Nicotinamide + | | |
| | | | CAETHG_0498 | Ribose 1-phosphate), ATP: acetate phosphotransferase (ATP + | | |
| | | | | H+ + Propionate --> ADP + Propionyl phosphate), | | |
| | | | | Cystathionine beta lyase (H2O + Cystathionine --> NH3 + | | |
| | | | | Pyruvate + Homocysteine) | | |
| 21 | Yield- | 3 | CAETHG_3359, | ATP: acetate phosphotransferase (ATP + H+ + Propionate --> | 0 | 0.02774 |
| | LMOMA | | CAETHG_3924, | ADP + Propionyl phosphate), D-Ribose 1,5-phosphomutase | | |
| | | | CAETHG_0498 | (Ribose 1-phosphate <=> ribose-5-phosphate), Cystathionine | | |
| | | | | beta lyase (H2O + Cystathionine --> NH3 + Pyruvate + | | |
| | | | | Homocysteine) | | |
| 22 | Yield- | 3 | CAETHG_1270, | AMP: pyrophosphate phosphoribosyltransferase (PPi + AMP <=> | 0 | 0.02774 |
| | LMOMA | | CAETHG_3359, | PRPP + Adenine), ATP: acetate phosphotransferase (ATP + | | |
| | | | CAETHG_0498 | H+ + Propionate --> ADP + Propionyl phosphate), | | |
| | | | | Cystathionine beta lyase (H2O + Cystathionine --> NH3 + | | |
| | | | | Pyruvate + Homocysteine) | | |
| 23 | Yield- | 3 | CAETHG_1371, | Adenosine 5′-monophosphate phosphohydrolase (H2O + | 0 | 0.02774 |
| | LMOMA | | CAETHG_3359, | AMP <=> Phosphate + H+ + Adenosine), ATP: acetate | | |
| | | | CAETHG_0498 | phosphotransferase (ATP + H+ + Propionate --> ADP + | | |
| | | | | Propionyl phosphate), Cystathionine beta lyase (H2O + | | |
| | | | | Cystathionine --> NH3 + Pyruvate + Homocysteine) | | |
| 24 | Yield- | 2 | CAETHG_3359, | ATP: acetate phosphotransferase (ATP + H+ + Propionate --> | 0 | 0.027712 |
| | LMOMA | | CAETHG_0498 | ADP + Propionyl phosphate), Cystathionine beta lyase (H2O + | | |
| | | | | Cystathionine --> NH3 + Pyruvate + Homocysteine) | | |
| 25 | Yield- | 3 | CAETHG_3021, | L-Arginine iminohydrolase (H2O + L-Arginine <=> NH3 + | 0 | 0.027688 |
| | LMOMA | | CAETHG_3359, | Citrulline), ATP: acetate phosphotransferase (ATP + H+ + | | |
| | | | CAETHG_3924 | Propionate --> ADP + Propionyl phosphate), D-Ribose 1,5- | | |
| | | | | phosphomutase (Ribose 1-phosphate <=> ribose-5- | | |
| | | | | phosphate) | | |
| 26 | Yield- | 2 | CAETHG_1270, | AMP: pyrophosphate phosphoribosyltransferase (PPi + AMP <=> | 0 | 0.027676 |
| | LMOMA | | CAETHG_3359 | PRPP + Adenine), ATP: acetate phosphotransferase (ATP + | | |
| | | | | H+ + Propionate --> ADP + Propionyl phosphate) | | |
| 27 | Yield- | 2 | CAETHG_0160, | N-Ribosylnicotinamide: orthophosphate ribosyltransferase | 0 | 0.027676 |
| | LMOMA | | CAETHG_3359 | (Phosphate + N-Ribosylnicotinamide <=> Nicotinamide + | | |
| | | | | Ribose 1-phosphate), ATP: acetate phosphotransferase (ATP + | | |
| | | | | H+ + Propionate --> ADP + Propionyl phosphate) | | |
| 28 | Yield- | 2 | CAETHG_1371, | Adenosine 5′-monophosphate phosphohydrolase (H2O + | 0 | 0.027676 |
| | LMOMA | | CAETHG_3359 | AMP <=> Phosphate + H+ + Adenosine), ATP: acetate | | |
| | | | | phosphotransferase (ATP + H+ + Propionate --> ADP + | | |
| | | | | Propionyl phosphate) | | |
| 29 | Yield- | 2 | CAETHG_3359, | ATP: acetate phosphotransferase (ATP + H+ + Propionate --> | 0 | 0.027676 |
| | LMOMA | | CAETHG_3924 | ADP + Propionyl phosphate), D-Ribose 1,5-phosphomutase | | |
| | | | | (Ribose 1-phosphate <=> ribose-5-phosphate) | | |
| 30 | Yield- | 2 | CAETHG_1270, | AMP: pyrophosphate phosphoribosyltransferase (PPi + AMP <=> | 0 | 0.027676 |
| | LMOMA | | CAETHG_3358 | PRPP + Adenine), Phosphate transacetylase (Phosphate + | | |
| | | | | Acetyl-CoA + H+ <=> CoA + Acetylphosphate) | | |
| 31 | Yield- | 2 | CAETHG_3021, | L-Arginine iminohydrolase (H2O + L-Arginine <=> NH3 + | 0 | 0.027656 |
| | LMOMA | | CAETHG_3359 | Citrulline), ATP: acetate phosphotransferase (ATP + H+ + | | |
| | | | | Propionate --> ADP + Propionyl phosphate) | | |
| 32 | Yield- | 1 | CAETHG_3359 | ATP: acetate phosphotransferase (ATP + H+ + Propionate --> | 0 | 0.027644 |
| | LMOMA | | | ADP + Propionyl phosphate) | | |
| 33 | Yield- | 1 | CAETHG_3358 | Phosphate transacetylase (Phosphate + Acetyl-CoA + H+ <=> | 0 | 0.027644 |
| | LMOMA | | | CoA + Acetylphosphate) | | |

### Example 13

This example describes disruptions for improved production of acetolactate in Wood-Ljungdahl microorganisms. Metabolic modeling experiments were performed as described in Example 1. Acetolactate is a native product of at least some Wood-Ljungdahl microorganisms.

| | | # | | | FVA | |
|---|---|---|---|---|---|---|
| | | Disrupted | Disrupted | | minimum | Fitness |
| # | Technique | genes | genes | Disrupted reactions | yield | score |
| 1 | Yield- | 5 | CAETHG_2751, | Citramalate synthase (H2O + Pyruvate + Acetyl-CoA <=> CoA + | 0.079303 | 0.19563 |
| | LMOMA | | CAETHG_2932, | Citramalate), Alpha-acetolactate decarboxylase (ALCTT --> | | |
| | | | CAETHG_3359, | CO2 + (R)-Acetoin), ATP: acetate phosphotransferase (ATP + | | |
| | | | CAETHG_3510, | H+ + Propionate --> ADP + Propionyl phosphate), 2,6- | | |
| | | | CAETHG_0498 | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate), Cystathionine beta | | |
| | | | | lyase (H2O + Cystathionine --> NH3 + Pyruvate + | | |
| | | | | Homocysteine) | | |
| 2 | Yield- | 5 | CAETHG_1371, | Adenosine 5′-monophosphate phosphohydrolase (H2O + AMP <=> | 0.077232 | 0.195495 |
| | LMOMA | | CAETHG_2932, | Phosphate + H+ + Adenosine), Alpha-acetolactate | | |
| | | | CAETHG_3359, | decarboxylase (ALCTT --> CO2 + (R)-Acetoin), ATP: acetate | | |
| | | | CAETHG_3510, | phosphotransferase (ATP + H+ + Propionate --> ADP + | | |
| | | | CAETHG_0498 | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 3 | Yield- | 5 | CAETHG_0160, | N-Ribosylnicotinamide: orthophosphate ribosyltransferase | 0.077232 | 0.195495 |
| | LMOMA | | CAETHG_2932, | (Phosphate + N-Ribosylnicotinamide <=> Nicotinamide + | | |
| | | | CAETHG_3359, | Ribose 1-phosphate), Alpha-acetolactate decarboxylase | | |
| | | | CAETHG_3510, | (ALCTT --> CO2 + (R)-Acetoin), ATP: acetate | | |
| | | | CAETHG_0498 | phosphotransferase (ATP + H+ + Propionate --> ADP + | | |
| | | | | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 4 | Yield- | 5 | CAETHG_2932, | Alpha-acetolactate decarboxylase (ALCTT --> CO2 + (R)- | 0.077232 | 0.195495 |
| | LMOMA | | CAETHG_3359, | Acetoin), ATP: acetate phosphotransferase (ATP + H+ + | | |
| | | | CAETHG_3510, | Propionate --> ADP + Propionyl phosphate), 2,6- | | |
| | | | CAETHG_3924, | Diaminoheptanedioate: 2-oxoglutarate aminotransferase | | |
| | | | CAETHG_0498 | (H2O + L-Glutamate + H+ + tetrahydrodipicolinate <-- 2- | | |
| | | | | Oxoglutarate + LL-2,6-Diaminopimelate), D-Ribose 1,5- | | |
| | | | | phosphomutase (Ribose 1-phosphate <=> ribose-5- | | |
| | | | | phosphate), Cystathionine beta lyase (H2O + Cystathionine --> | | |
| | | | | NH3 + Pyruvate + Homocysteine) | | |
| 5 | Yield- | 5 | CAETHG_1270, | AMP: pyrophosphate phosphoribosyltransferase (PPi + AMP <=> | 0.077232 | 0.195495 |
| | LMOMA | | CAETHG_2932, | PRPP + Adenine), Alpha-acetolactate decarboxylase | | |
| | | | CAETHG_3359, | (ALCTT --> CO2 + (R)-Acetoin), ATP: acetate | | |
| | | | CAETHG_3510, | phosphotransferase (ATP + H+ + Propionate --> ADP + | | |
| | | | CAETHG_0498 | Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 6 | Yield- | 4 | CAETHG_2721, | Phosphoenolpyruvate carboxykinase (PPCK) (ATP + | 0 | 0.16721 |
| | LMOMA | | CAETHG_2753, | Oxaloacetate + H+ --> ADP + CO2 + Phosphoenolpyruvate), | | |
| | | | CAETHG_3358, | Isocitrate dehydrogenase (NAD + Isocitrate <=> NADH + CO2 + | | |
| | | | CAETHG_0686 | 2-Oxoglutarate + H+), Phosphate transacetylase (Phosphate + | | |
| | | | | Acetyl-CoA + H+ <=> CoA + Acetylphosphate), L-Threonine | | |
| | | | | acetaldehyde-lyase (L-Threonine --> Glycine + Acetaldehyde) | | |
| 7 | Yield- | 4 | CAETHG_2753, | Isocitrate dehydrogenase (NAD + Isocitrate <=> NADH + CO2 + | 0 | 0.16456 |
| | LMOMA | | CAETHG_3293, | 2-Oxoglutarate + H+), Cysteine desulfhydrase (H2O + L- | | |
| | | | CAETHG_3358, | Cysteine <=> NH3 + Pyruvate + H2S), Phosphate | | |
| | | | CAETHG_0686 | transacetylase (Phosphate + Acetyl-CoA + H+ <=> CoA + | | |
| | | | | Acetylphosphate), L-Threonine acetaldehyde-lyase (L- | | |
| | | | | Threonine --> Glycine + Acetaldehyde) | | |
| 8 | Yield- | 4 | CAETHG_3358, | Phosphate transacetylase (Phosphate + Acetyl-CoA + H+ <=> | 0 | 0.16301 |
| | LMOMA | | CAETHG_3510, | CoA + Acetylphosphate), 2,6-Diaminoheptanedioate: 2- | | |
| | | | CAETHG_3924, | oxoglutarate aminotransferase (H2O + L-Glutamate + H+ + | | |
| | | | CAETHG_0498 | tetrahydrodipicolinate <-- 2-Oxoglutarate + LL-2,6- | | |
| | | | | Diaminopimelate), D-Ribose 1,5-phosphomutase (Ribose 1- | | |
| | | | | phosphate <=> ribose-5-phosphate), Cystathionine beta lyase | | |
| | | | | (H2O + Cystathionine --> NH3 + Pyruvate + Homocysteine) | | |
| 9 | Yield- | 3 | CAETHG_3358, | Phosphate transacetylase (Phosphate + Acetyl-CoA + H+ <=> | 0 | 0.162785 |
| | LMOMA | | CAETHG_3510, | CoA + Acetylphosphate), 2,6-Diaminoheptanedioate: 2- | | |
| | | | CAETHG_0498 | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 10 | Yield- | 3 | CAETHG_3358, | Phosphate transacetylase (Phosphate + Acetyl-CoA + H+ <=> | 0 | 0.16169 |
| | LMOMA | | CAETHG_3510, | CoA + Acetylphosphate), 2,6-Diaminoheptanedioate: 2- | | |
| | | | CAETHG_3924 | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), D-Ribose 1,5-phosphomutase (Ribose 1- | | |
| | | | | phosphate <=> ribose-5-phosphate) | | |
| 11 | Yield- | 3 | CAETHG_1371, | Adenosine 5′-monophosphate phosphohydrolase (H2O + AMP <=> | 0 | 0.16169 |
| | LMOMA | | CAETHG_3358, | Phosphate + H+ + Adenosine), Phosphate transacetylase | | |
| | | | CAETHG_3510 | (Phosphate + Acetyl-CoA + H+ <=> CoA + Acetylphosphate), | | |
| | | | | 2,6-Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 12 | Yield- | 3 | CAETHG_0160, | N-Ribosylnicotinamide: orthophosphate ribosyltransferase | 0 | 0.16169 |
| | LMOMA | | CAETHG_3358, | (Phosphate + N-Ribosylnicotinamide <=> Nicotinamide + | | |
| | | | CAETHG_3510 | Ribose 1-phosphate), Phosphate transacetylase (Phosphate + | | |
| | | | | Acetyl-CoA + H+ <=> CoA + Acetylphosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 13 | Yield- | 3 | CAETHG_2932, | Alpha-acetolactate decarboxylase (ALCTT --> CO2 + (R)- | 0.077231 | 0.15909 |
| | LMOMA | | CAETHG_3358, | Acetoin), Phosphate transacetylase (Phosphate + Acetyl-CoA + | | |
| | | | CAETHG_3510 | H+ <=> CoA + Acetylphosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 14 | Yield- | 3 | CAETHG_3293, | Cysteine desulfhydrase (H2O + L-Cysteine <=> NH3 + Pyruvate + | 0 | 0.15344 |
| | LMOMA | | CAETHG_3358, | H2S), Phosphate transacetylase (Phosphate + Acetyl-CoA + | | |
| | | | CAETHG_3510 | H+ <=> CoA + Acetylphosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 15 | Yield- | 3 | CAETHG_3358, | Phosphate transacetylase (Phosphate + Acetyl-CoA + H+ <=> | 0 | 0.150755 |
| | LMOMA | | CAETHG_3510, | CoA + Acetylphosphate), 2,6-Diaminoheptanedioate: 2- | | |
| | | | CAETHG_0909 | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Prephenate: NAD+ | | |
| | | | | oxidoreductase(decarboxylating) (NAD + Prephenate --> | | |
| | | | | NADH + CO2 + p-hydroxyphenylpyruvate) | | |
| 16 | Yield- | 3 | CAETHG_3021, | L-Arginine iminohydrolase (H2O + L-Arginine <=> NH3 + | 0 | 0.15038 |
| | LMOMA | | CAETHG_3358, | Citrulline), Phosphate transacetylase (Phosphate + Acetyl-CoA + | | |
| | | | CAETHG_3510 | H+ <=> CoA + Acetylphosphate), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 17 | Yield- | 2 | CAETHG_3359, | ATP: acetate phosphotransferase (ATP + H+ + Propionate --> | 0 | 0.150325 |
| | LMOMA | | CAETHG_3510 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 18 | Yield- | 2 | CAETHG_3358, | Phosphate transacetylase (Phosphate + Acetyl-CoA + H+ <=> | 0 | 0.150325 |
| | LMOMA | | CAETHG_3510 | CoA + Acetylphosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 19 | Yield- | 4 | CAETHG_0160, | N-Ribosylnicotinamide: orthophosphate ribosyltransferase | 0 | 0.13579 |
| | LMOMA | | CAETHG_2753, | (Phosphate + N-Ribosylnicotinamide <=> Nicotinamide + | | |
| | | | CAETHG_3358, | Ribose 1-phosphate), Isocitrate dehydrogenase (NAD+ | | |
| | | | CAETHG_0686 | Isocitrate <=> NADH + CO2 + 2-Oxoglutarate + H+), Phosphate | | |
| | | | | transacetylase (Phosphate + Acetyl-CoA + H+ <=> CoA + | | |
| | | | | Acetylphosphate), L-Threonine acetaldehyde-lyase (L- | | |
| | | | | Threonine --> Glycine + Acetaldehyde) | | |
| 20 | Yield- | 3 | CAETHG_2753, | Isocitrate dehydrogenase (NAD + Isocitrate <=> NADH + CO2 + | 0 | 0.1302 |
| | LMOMA | | CAETHG_3358, | 2-Oxoglutarate + H+), Phosphate transacetylase (Phosphate + | | |
| | | | CAETHG_0686 | Acetyl-CoA + H+ <=> CoA + Acetylphosphate), L-Threonine | | |
| | | | | acetaldehyde-lyase (L-Threonine --> Glycine + Acetaldehyde) | | |
| 21 | Yield- | 3 | CAETHG_2721, | Phosphoenolpyruvate carboxykinase (PPCK) (ATP + | 0 | 0.127735 |
| | LMOMA | | CAETHG_3358, | Oxaloacetate + H+ --> ADP + CO2 + Phosphoenolpyruvate), | | |
| | | | CAETHG_0686 | Phosphate transacetylase (Phosphate + Acetyl-CoA + H+ <=> | | |
| | | | | CoA + Acetylphosphate), L-Threonine acetaldehyde-lyase (L- | | |
| | | | | Threonine --> Glycine + Acetaldehyde) | | |
| 22 | Yield- | 2 | CAETHG_2721, | Phosphoenolpyruvate carboxykinase (PPCK) (ATP + | 0 | 0.12009 |
| | LMOMA | | CAETHG_3358 | Oxaloacetate + H+ --> ADP + CO2 + Phosphoenolpyruvate), | | |
| | | | | Phosphate transacetylase (Phosphate + Acetyl-CoA + H+ <=> | | |
| | | | | CoA + Acetylphosphate) | | |
| 23 | Yield- | 3 | CAETHG_1371, | Adenosine 5′-monophosphate phosphohydrolase (H2O + AMP <=> | 0 | 0.110555 |
| | LMOMA | | CAETHG_3358, | Phosphate + H+ + Adenosine), Phosphate transacetylase | | |
| | | | CAETHG_0686 | (Phosphate + Acetyl-CoA + H+ <=> CoA + Acetylphosphate), L- | | |
| | | | | Threonine acetaldehyde-lyase (L-Threonine --> Glycine + | | |
| | | | | Acetaldehyde) | | |
| 24 | Yield- | 3 | CAETHG_0160, | N-Ribosylnicotinamide: orthophosphate ribosyltransferase | 0 | 0.110555 |
| | LMOMA | | CAETHG_3358, | (Phosphate + N-Ribosylnicotinamide <=> Nicotinamide + | | |
| | | | CAETHG_0686 | Ribose 1-phosphate), Phosphate transacetylase (Phosphate + | | |
| | | | | Acetyl-CoA + H+ <=> CoA + Acetylphosphate), L-Threonine | | |
| | | | | acetaldehyde-lyase (L-Threonine --> Glycine + Acetaldehyde) | | |
| 25 | Yield- | 3 | CAETHG_3358, | Phosphate transacetylase (Phosphate + Acetyl-CoA + H+ <=> | 0 | 0.10917 |
| | LMOMA | | CAETHG_0498, | CoA + Acetylphosphate), Cystathionine beta lyase (H2O + | | |
| | | | CAETHG_0686 | Cystathionine --> NH3 + Pyruvate + Homocysteine), L- | | |
| | | | | Threonine acetaldehyde-lyase (L-Threonine --> Glycine + | | |
| | | | | Acetaldehyde) | | |
| 26 | Yield- | 3 | CAETHG_3021, | L-Arginine iminohydrolase (H2O + L-Arginine <=> NH3 + | 0 | 0.10734 |
| | LMOMA | | CAETHG_3358, | Citrulline), Phosphate transacetylase (Phosphate + Acetyl-CoA + | | |
| | | | CAETHG_0686 | H+ <=> CoA + Acetylphosphate), L-Threonine acetaldehyde- | | |
| | | | | lyase (L-Threonine --> Glycine + Acetaldehyde) | | |
| 27 | Yield- | 3 | CAETHG_3164, | UMP: pyrophosphate phosphoribosyltransferase (Uracil + | 0 | 0.10725 |
| | LMOMA | | CAETHG_3358, | PRPP --> PPi + UMP), Phosphate transacetylase (Phosphate + | | |
| | | | CAETHG_0686 | Acetyl-CoA + H+ <=> CoA + Acetylphosphate), L-Threonine | | |
| | | | | acetaldehyde-lyase (L-Threonine --> Glycine + Acetaldehyde) | | |
| 28 | Yield- | 2 | CAETHG_3358, | Phosphate transacetylase (Phosphate + Acetyl-CoA + H+ <=> | 0 | 0.10722 |
| | LMOMA | | CAETHG_0686 | CoA + Acetylphosphate), L-Threonine acetaldehyde-lyase (L- | | |
| | | | | Threonine --> Glycine + Acetaldehyde) | | |
| 29 | Yield- | 2 | CAETHG_3359, | ATP: acetate phosphotransferase (ATP + H+ + Propionate --> | 0 | 0.10722 |
| | LMOMA | | CAETHG_0686 | ADP + Propionyl phosphate), L-Threonine acetaldehyde-lyase | | |
| | | | | (L-Threonine --> Glycine + Acetaldehyde) | | |
| 30 | Yield- | 3 | CAETHG_3358, | Phosphate transacetylase (Phosphate + Acetyl-CoA + H+ <=> | 0 | 0.104905 |
| | LMOMA | | CAETHG_3924, | CoA + Acetylphosphate), D-Ribose 1,5-phosphomutase | | |
| | | | CAETHG_0498 | (Ribose 1-phosphate <=> ribose-5-phosphate), Cystathionine | | |
| | | | | beta lyase (H2O + Cystathionine --> NH3 + Pyruvate + | | |
| | | | | Homocysteine) | | |
| 31 | Yield- | 3 | CAETHG_1270, | AMP: pyrophosphate phosphoribosyltransferase (PPi + AMP <=> | 0 | 0.104905 |
| | LMOMA | | CAETHG_3358, | PRPP + Adenine), Phosphate transacetylase (Phosphate + | | |
| | | | CAETHG_0498 | Acetyl-CoA + H+ <=> CoA + Acetylphosphate), Cystathionine | | |
| | | | | beta lyase (H2O + Cystathionine --> NH3 + Pyruvate + | | |
| | | | | Homocysteine) | | |
| 32 | Yield- | 3 | CAETHG_0160, | N-Ribosylnicotinamide: orthophosphate ribosyltransferase | 0 | 0.104905 |
| | LMOMA | | CAETHG_3358, | (Phosphate + N-Ribosylnicotinamide <=> Nicotinamide + | | |
| | | | CAETHG_0498 | Ribose 1-phosphate), Phosphate transacetylase (Phosphate + | | |
| | | | | Acetyl-CoA + H+ <=> CoA + Acetylphosphate), Cystathionine | | |
| | | | | beta lyase (H2O + Cystathionine --> NH3 + Pyruvate + | | |
| | | | | Homocysteine) | | |
| 33 | Yield- | 3 | CAETHG_3299, | 2-Deoxy-D-ribose-5-phosphate acetaldehyde-lyase | 0 | 0.10331 |
| | LMOMA | | CAETHG_3358, | (deoxyribose-5-phosphate <=> Acetaldehyde + | | |
| | | | CAETHG_0498 | Glyceraldehyde3-phosphate), Phosphate transacetylase | | |
| | | | | (Phosphate + Acetyl-CoA + H+ <=> CoA + Acetylphosphate), | | |
| | | | | Cystathionine beta lyase (H2O + Cystathionine --> NH3 + | | |
| | | | | Pyruvate + Homocysteine) | | |
| 34 | Yield- | 3 | CAETHG_3021, | L-Arginine iminohydrolase (H2O + L-Arginine <=> NH3 + | 0 | 0.10228 |
| | LMOMA | | CAETHG_3358, | Citrulline), Phosphate transacetylase (Phosphate + Acetyl-CoA + | | |
| | | | CAETHG_0498 | H+ <=> CoA + Acetylphosphate), Cystathionine beta lyase | | |
| | | | | (H2O + Cystathionine --> NH3 + Pyruvate + Homocysteine) | | |
| 35 | Yield- | 3 | CAETHG_3164, | UMP: pyrophosphate phosphoribosyltransferase (Uracil + | 0 | 0.10218 |
| | LMOMA | | CAETHG_3358, | PRPP --> PPi + UMP), Phosphate transacetylase (Phosphate + | | |
| | | | CAETHG_0498 | Acetyl-CoA + H+ <=> CoA + Acetylphosphate), Cystathionine | | |
| | | | | beta lyase (H2O + Cystathionine --> NH3 + Pyruvate + | | |
| | | | | Homocysteine) | | |
| 36 | Yield- | 2 | CAETHG_3358, | Phosphate transacetylase (Phosphate + Acetyl-CoA + H+ <=> | 0 | 0.102155 |
| | LMOMA | | CAETHG_0498 | CoA + Acetylphosphate), Cystathionine beta lyase (H2O + | | |
| | | | | Cystathionine --> NH3 + Pyruvate + Homocysteine) | | |
| 37 | Yield- | 2 | CAETHG_1371, | Adenosine 5′-monophosphate phosphohydrolase (H2O + AMP <=> | 0 | 0.10147 |
| | LMOMA | | CAETHG_3358 | Phosphate + H+ + Adenosine), Phosphate transacetylase | | |
| | | | | (Phosphate + Acetyl-CoA + H+ <=> CoA + Acetylphosphate) | | |
| 38 | Yield- | 2 | CAETHG_1270, | AMP: pyrophosphate phosphoribosyltransferase (PPi + AMP <=> | 0 | 0.10147 |
| | LMOMA | | CAETHG_3358 | PRPP + Adenine), Phosphate transacetylase (Phosphate + | | |
| | | | | Acetyl-CoA + H+ <=> CoA + Acetylphosphate) | | |
| 39 | Yield- | 2 | CAETHG_3358, | Phosphate transacetylase (Phosphate + Acetyl-CoA + H+ <=> | 0 | 0.10147 |
| | LMOMA | | CAETHG_3924 | CoA + Acetylphosphate), D-Ribose 1,5-phosphomutase | | |
| | | | | (Ribose 1-phosphate <=> ribose-5-phosphate) | | |
| 40 | Yield- | 2 | CAETHG_0160, | N-Ribosylnicotinamide: orthophosphate ribosyltransferase | 0 | 0.10147 |
| | LMOMA | | CAETHG_3358 | (Phosphate + N-Ribosylnicotinamide <=> Nicotinamide + | | |
| | | | | Ribose 1-phosphate), Phosphate transacetylase (Phosphate + | | |
| | | | | Acetyl-CoA + H+ <=> CoA + Acetylphosphate) | | |
| 41 | Yield- | 2 | CAETHG_3299, | 2-Deoxy-D-ribose-5-phosphate acetaldehyde-lyase | 0 | 0.09974 |
| | LMOMA | | CAETHG_3358 | (deoxyribose-5-phosphate <=> Acetaldehyde + | | |
| | | | | Glyceraldehyde3-phosphate), Phosphate transacetylase | | |
| | | | | (Phosphate + Acetyl-CoA + H+ <=> CoA + Acetylphosphate) | | |
| 42 | Yield- | 2 | CAETHG_2475, | dGTP triphosphohydrolase (H2O + dGTP --> H+ + | 0 | 0.099725 |
| | LMOMA | | CAETHG_3358 | Deoxyguanosine + Triphosphate), Phosphate transacetylase | | |
| | | | | (Phosphate + Acetyl-CoA + H+ <=> CoA + Acetylphosphate) | | |
| 43 | Yield- | 2 | CAETHG_3021, | L-Arginine iminohydrolase (H2O + L-Arginine <=> NH3 + | 0 | 0.0988 |
| | LMOMA | | CAETHG_3358 | Citrulline), Phosphate transacetylase (Phosphate + Acetyl-CoA + | | |
| | | | | H+ <=> CoA + Acetylphosphate) | | |
| 44 | Yield- | 2 | CAETHG_3164, | UMP: pyrophosphate phosphoribosyltransferase (Uracil + | 0 | 0.098685 |
| | LMOMA | | CAETHG_3358 | PRPP --> PPi + UMP), Phosphate transacetylase (Phosphate + | | |
| | | | | Acetyl-CoA + H+ <=> CoA + Acetylphosphate) | | |
| 45 | Yield- | 2 | CAETHG_2107, | Potassium uptake (K+_ext <=> K+), Phosphate transacetylase | 0 | 0.098675 |
| | LMOMA | | CAETHG_3358 | (Phosphate + Acetyl-CoA + H+ <=> CoA + Acetylphosphate) | | |
| 46 | Yield- | 1 | CAETHG_3358 | Phosphate transacetylase (Phosphate + Acetyl-CoA + H+ <=> | 0 | 0.09867 |
| | LMOMA | | | CoA + Acetylphosphate) | | |
| 47 | Yield- | 1 | CAETHG_3359 | ATP: acetate phosphotransferase (ATP + H+ + Propionate --> | 0 | 0.09867 |
| | LMOMA | | | ADP + Propionyl phosphate) | | |
| 48 | Yield- | 2 | CAETHG_2932, | Alpha-acetolactate decarboxylase (ALCTT --> CO2 + (R)- | 0 | 0.048115 |
| | LMOMA | | CAETHG_3924 | Acetoin), D-Ribose 1,5-phosphomutase (Ribose 1-phosphate <=> | | |
| | | | | ribose-5-phosphate) | | |
| 49 | Yield- | 1 | CAETHG_2932 | Alpha-acetolactate decarboxylase (ALCTT --> CO2 + (R)- | 0 | 0.04552 |
| | LMOMA | | | Acetoin) | | |
| 50 | Yield- | 2 | CAETHG_2721, | Phosphoenolpyruvate carboxykinase (PPCK) (ATP + | 0 | 0.021365 |
| | LMOMA | | CAETHG_0909 | Oxaloacetate + H+ --> ADP + CO2 + Phosphoenolpyruvate), | | |
| | | | | Prephenate: NAD+ oxidoreductase(decarboxylating) (NAD+ | | |
| | | | | Prephenate --> NADH + CO2 + p-hydroxyphenylpyruvate) | | |
| 51 | Yield- | 2 | CAETHG_0248, | L-lactate reversible transport via proton symport (H+_ext + L- | 0 | 0.021235 |
| | LMOMA | | CAETHG_2721 | Lactate_ext <=> H+ + L-Lactate), Phosphoenolpyruvate | | |
| | | | | carboxykinase (PPCK) (ATP + Oxaloacetate + H+ --> ADP + | | |
| | | | | CO2 + Phosphoenolpyruvate) | | |
| 52 | Yield- | 2 | CAETHG_1270, | AMP: pyrophosphate phosphoribosyltransferase (PPi + AMP <=> | 0 | 0.02115 |
| | LMOMA | | CAETHG_2721 | PRPP + Adenine), Phosphoenolpyruvate carboxykinase | | |
| | | | | (PPCK) (ATP + Oxaloacetate + H+ --> ADP + CO2 + | | |
| | | | | Phosphoenolpyruvate) | | |
| 53 | Yield- | 2 | CAETHG_0160, | N-Ribosylnicotinamide: orthophosphate ribosyltransferase | 0 | 0.02115 |
| | LMOMA | | CAETHG_2721 | (Phosphate + N-Ribosylnicotinamide <=> Nicotinamide + | | |
| | | | | Ribose 1-phosphate), Phosphoenolpyruvate carboxykinase | | |
| | | | | (PPCK) (ATP + Oxaloacetate + H+ --> ADP + CO2 + | | |
| | | | | Phosphoenolpyruvate) | | |
| 54 | Yield- | 2 | CAETHG_2721, | Phosphoenolpyruvate carboxykinase (PPCK) (ATP + | 0 | 0.02115 |
| | LMOMA | | CAETHG_3924 | Oxaloacetate + H+ --> ADP + CO2 + Phosphoenolpyruvate), D- | | |
| | | | | Ribose 1,5-phosphomutase (Ribose 1-phosphate <=> ribose-5- | | |
| | | | | phosphate) | | |
| 55 | Yield- | 2 | CAETHG_2721, | Phosphoenolpyruvate carboxykinase (PPCK) (ATP + | 0 | 0.021085 |
| | LMOMA | | CAETHG_2751 | Oxaloacetate + H+ --> ADP + CO2 + Phosphoenolpyruvate), | | |
| | | | | Citramalate synthase (H2O + Pyruvate + Acetyl-CoA <=> CoA + | | |
| | | | | Citramalate) | | |
| 56 | Yield- | 1 | CAETHG_0160 | N-Ribosylnicotinamide: orthophosphate ribosyltransferase | 0 | 0.020825 |
| | LMOMA | | | (Phosphate + N-Ribosylnicotinamide <=> Nicotinamide + | | |
| | | | | Ribose 1-phosphate) | | |
| 57 | Yield- | 1 | CAETHG_1270 | AMP: pyrophosphate phosphoribosyltransferase (PPi + AMP <=> | 0 | 0.020825 |
| | LMOMA | | | PRPP + Adenine) | | |
| 58 | Yield- | 1 | CAETHG_1371 | Adenosine 5′-monophosphate phosphohydrolase (H2O + AMP <=> | 0 | 0.020825 |
| | LMOMA | | | Phosphate + H+ + Adenosine) | | |
| 59 | Yield- | 1 | CAETHG_3924 | D-Ribose 1,5-phosphomutase (Ribose 1-phosphate <=> | 0 | 0.020825 |
| | LMOMA | | | ribose-5-phosphate) | | |
| 60 | Yield- | 2 | CAETHG_2721, | Phosphoenolpyruvate carboxykinase (PPCK) (ATP + | 0 | 0.02046 |
| | LMOMA | | CAETHG_3021 | Oxaloacetate + H+ --> ADP + CO2 + Phosphoenolpyruvate), L- | | |
| | | | | Arginine iminohydrolase (H2O + L-Arginine <=> NH3 + | | |
| | | | | Citrulline) | | |
| 61 | Yield- | 2 | CAETHG_2107, | Potassium uptake (K+_ext <=> K+), Phosphoenolpyruvate | 0 | 0.020425 |
| | LMOMA | | CAETHG_2721 | carboxykinase (PPCK) (ATP + Oxaloacetate + H+ --> ADP + | | |
| | | | | CO2 + Phosphoenolpyruvate) | | |
| 62 | Yield- | 1 | CAETHG_2721 | Phosphoenolpyruvate carboxykinase (PPCK) (ATP + | 0 | 0.02042 |
| | LMOMA | | | Oxaloacetate + H+ --> ADP + CO2 + Phosphoenolpyruvate) | | |
| 63 | Yield- | 1 | CAETHG_2751 | Citramalate synthase (H2O + Pyruvate + Acetyl-CoA <=> CoA + | 0 | 0.020095 |
| | LMOMA | | | Citramalate) | | |
| 64 | Yield- | 1 | CAETHG_3021 | L-Arginine iminohydrolase (H2O + L-Arginine <=> NH3 + | 0 | 0.020045 |
| | LMOMA | | | Citrulline) | | |

### Example 14

This example describes disruptions for improved production of isoprene in Wood-Ljungdahl microorganisms. Metabolic modeling experiments were performed as described in Example 1. Production of isoprene in Wood-Ljungdahl microorganisms is described, e.g., in WO 2013/180584. The following pathway was used to model isoprene production herein: 2.0 Acetyl-CoA-->CoA+Acetoacetyl-CoA; 2.0 NADH+2.0H++(S)-3-Hydroxy-3-methylglutaryl-CoA-->2.0 NAD+CoA+(R)-Mevalonate; ATP+(R)-Mevalonate-->ADP+(R)-5-Phosphomevalonate; ATP+(R)-5-Phosphomevalonate-->ADP+(R)-5-Diphosphomevalonate; ATP+(R)-5-Diphosphomevalonate-->ADP+Phosphate+C02+Isopentenyldiphosphate; Isopentenyldiphosphate-->DMAPP; DMAPP-->PPi+Isoprene; H₂O+Acetyl-CoA+Acetoacetyl-CoA-->CoA+(S)-3-Hydroxy-3-methylglutaryl-CoA; Isoprene-->Isoprene_ext. These disruptions would also be useful for improving production of IPP/DMAPP and/or products downstream of IPP/DMAPP, such as famesene and other terpenoids.

| | | # | | | FVA | |
|---|---|---|---|---|---|---|
| | | Disrupted | Disrupted | | minimum | Fitness |
| # | Technique | genes | genes | Disrupted reactions | yield | score |
| 1 | Yield- | 5 | CAETHG_2062, | Phosphoenolpyruvate carboxykinase (PPCK) (ATP + Oxaloacetate + | 0 | 0.05024 |
| | LMOMA | | CAETHG_2479, | H+ --> ADP + CO2 + Phosphoenolpyruvate), Alpha-acetolactate | | |
| | | | CAETHG_2721, | decarboxylase (ALCTT --> CO2 + (R)-Acetoin), Phosphate | | |
| | | | CAETHG_2932, | transacetylase (Phosphate + Acetyl-CoA + H+ <=> CoA + | | |
| | | | CAETHG_3358 | Acetylphosphate) | | |
| 2 | Yield- | 5 | CAETHG_0160, | N-Ribosylnicotinamide: orthophosphate ribosyltransferase | 0 | 0.049965 |
| | LMOMA | | CAETHG_2721, | (Phosphate + N-Ribosylnicotinamide <=> Nicotinamide + Ribose 1- | | |
| | | | CAETHG_2932, | phosphate), Phosphoenolpyruvate carboxykinase (PPCK) (ATP + | | |
| | | | CAETHG_3358, | Oxaloacetate + H+ --> ADP + CO2 + Phosphoenolpyruvate), Alpha- | | |
| | | | CAETHG_0498 | acetolactate decarboxylase (ALCTT --> CO2 + (R)-Acetoin), | | |
| | | | | Phosphate transacetylase (Phosphate + Acetyl-CoA + H+ <=> CoA + | | |
| | | | | Acetylphosphate), Cystathionine beta lyase (H2O + Cystathionine -- > | | |
| | | | | NH3 + Pyruvate + Homocysteine) | | |
| 3 | Yield- | 5 | CAETHG_0160, | N-Ribosylnicotinamide: orthophosphate ribosyltransferase | 0 | 0.04994 |
| | LMOMA | | CAETHG_2721, | (Phosphate + N-Ribosylnicotinamide <=> Nicotinamide + Ribose 1- | | |
| | | | CAETHG_2932, | phosphate), Phosphoenolpyruvate carboxykinase (PPCK) (ATP + | | |
| | | | CAETHG_3021, | Oxaloacetate + H+ --> ADP + CO2 + Phosphoenolpyruvate), Alpha- | | |
| | | | CAETHG_3358 | acetolactate decarboxylase (ALCTT --> CO2 + (R)-Acetoin), L- | | |
| | | | | Arginine iminohydrolase (H2O + L-Arginine <=> NH3 + Citrulline), | | |
| | | | | Phosphate transacetylase (Phosphate + Acetyl-CoA + H+ <=> CoA + | | |
| | | | | Acetylphosphate) | | |
| 4 | Yield- | 5 | CAETHG_2721, | Phosphoenolpyruvate carboxykinase (PPCK) (ATP + Oxaloacetate + | 0 | 0.04994 |
| | LMOMA | | CAETHG_2932, | H+ --> ADP + CO2 + Phosphoenolpyruvate), Alpha-acetolactate | | |
| | | | CAETHG_3021, | decarboxylase (ALCTT --> CO2 + (R)-Acetoin), L-Arginine | | |
| | | | CAETHG_3358, | iminohydrolase (H2O + L-Arginine <=> NH3 + Citrulline), Phosphate | | |
| | | | CAETHG_3924 | transacetylase (Phosphate + Acetyl-CoA + H+ <=> CoA + | | |
| | | | | Acetylphosphate), D-Ribose 1,5-phosphomutase (Ribose 1- | | |
| | | | | phosphate <=> ribose-5-phosphate) | | |
| 5 | Yield- | 4 | CAETHG_2721, | Phosphoenolpyruvate carboxykinase (PPCK) (ATP + Oxaloacetate + | 0 | 0.04993 |
| | LMOMA | | CAETHG_2932, | H+ --> ADP + CO2 + Phosphoenolpyruvate), Alpha-acetolactate | | |
| | | | CAETHG_3358, | decarboxylase (ALCTT --> CO2 + (R)-Acetoin), Phosphate | | |
| | | | CAETHG_3924 | transacetylase (Phosphate + Acetyl-CoA + H+ <=> CoA + | | |
| | | | | Acetylphosphate), D-Ribose 1,5-phosphomutase (Ribose 1- | | |
| | | | | phosphate <=> ribose-5-phosphate) | | |
| 6 | Yield- | 4 | CAETHG_0160, | N-Ribosylnicotinamide: orthophosphate ribosyltransferase | 0 | 0.04993 |
| | LMOMA | | CAETHG_2721, | (Phosphate + N-Ribosylnicotinamide <=> Nicotinamide + Ribose 1- | | |
| | | | CAETHG_2932, | phosphate), Phosphoenolpyruvate carboxykinase (PPCK) (ATP + | | |
| | | | CAETHG_3358 | Oxaloacetate + H+ --> ADP + CO2 + Phosphoenolpyruvate), Alpha- | | |
| | | | | acetolactate decarboxylase (ALCTT --> CO2 + (R)-Acetoin), | | |
| | | | | Phosphate transacetylase (Phosphate + Acetyl-CoA + H+ <=> CoA + | | |
| | | | | Acetylphosphate) | | |
| 7 | Yield- | 4 | CAETHG_0160, | N-Ribosylnicotinamide: orthophosphate ribosyltransferase | 0 | 0.04993 |
| | LMOMA | | CAETHG_2721, | (Phosphate + N-Ribosylnicotinamide <=> Nicotinamide + Ribose 1- | | |
| | | | CAETHG_2932, | phosphate), Phosphoenolpyruvate carboxykinase (PPCK) (ATP + | | |
| | | | CAETHG_3359 | Oxaloacetate + H+ --> ADP + CO2 + Phosphoenolpyruvate), Alpha- | | |
| | | | | acetolactate decarboxylase (ALCTT --> CO2 + (R)-Acetoin), | | |
| | | | | ATP: acetate phosphotransferase (ATP + H+ + Propionate --> ADP + | | |
| | | | | Propionyl phosphate) | | |
| 8 | Yield- | 4 | CAETHG_1270, | AMP: pyrophosphate phosphoribosyltransferase (PPi + AMP <=> | 0 | 0.04991 |
| | LMOMA | | CAETHG_2721, | PRPP + Adenine), Phosphoenolpyruvate carboxykinase (PPCK) | | |
| | | | CAETHG_2932, | (ATP + Oxaloacetate + H+ --> ADP + CO2 + Phosphoenolpyruvate), | | |
| | | | CAETHG_3358 | Alpha-acetolactate decarboxylase (ALCTT --> CO2 + (R)-Acetoin), | | |
| | | | | Phosphate transacetylase (Phosphate + Acetyl-CoA + H+ <=> CoA + | | |
| | | | | Acetylphosphate) | | |
| 9 | Yield- | 3 | CAETHG_2721, | Phosphoenolpyruvate carboxykinase (PPCK) (ATP + Oxaloacetate + | 0 | 0.049595 |
| | LMOMA | | CAETHG_2932, | H+ --> ADP + CO2 + Phosphoenolpyruvate), Alpha-acetolactate | | |
| | | | CAETHG_3358 | decarboxylase (ALCTT --> CO2 + (R)-Acetoin), Phosphate | | |
| | | | | transacetylase (Phosphate + Acetyl-CoA + H+ <=> CoA + | | |
| | | | | Acetylphosphate) | | |
| 10 | Yield- | 4 | CAETHG_2932, | Alpha-acetolactate decarboxylase (ALCTT --> CO2 + (R)-Acetoin), | 0 | 0.04102 |
| | LMOMA | | CAETHG_3358, | Phosphate transacetylase (Phosphate + Acetyl-CoA + H+ <=> CoA + | | |
| | | | CAETHG_3510, | Acetylphosphate), 2,6-Diaminoheptanedioate: 2-oxoglutarate | | |
| | | | CAETHG_3924 | aminotransferase (H2O + L-Glutamate + H+ + | | |
| | | | | tetrahydrodipicolinate <-- 2-Oxoglutarate + LL-2,6- | | |
| | | | | Diaminopimelate), D-Ribose 1,5-phosphomutase (Ribose 1- | | |
| | | | | phosphate <=> ribose-5-phosphate) | | |
| 11 | Yield- | 4 | CAETHG_1270, | AMP: pyrophosphate phosphoribosyltransferase (PPi + AMP <=> | 0 | 0.04102 |
| | LMOMA | | CAETHG_2932, | PRPP + Adenine), Alpha-acetolactate decarboxylase (ALCTT --> | | |
| | | | CAETHG_3358, | CO2 + (R)-Acetoin), Phosphate transacetylase (Phosphate + Acetyl- | | |
| | | | CAETHG_3510 | CoA + H+ <=> CoA + Acetylphosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 12 | Yield- | 4 | CAETHG_2932, | Alpha-acetolactate decarboxylase (ALCTT --> CO2 + (R)-Acetoin), L- | 0 | 0.04041 |
| | LMOMA | | CAETHG_3021, | Arginine iminohydrolase (H2O + L-Arginine <=> NH3 + Citrulline), | | |
| | | | CAETHG_3358, | Phosphate transacetylase (Phosphate + Acetyl-CoA + H+ <=> CoA + | | |
| | | | CAETHG_3510 | Acetylphosphate), 2,6-Diaminoheptanedioate: 2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 13 | Yield- | 3 | CAETHG_2932, | Alpha-acetolactate decarboxylase (ALCTT --> CO2 + (R)-Acetoin), | 0 | 0.040385 |
| | LMOMA | | CAETHG_3359, | ATP: acetate phosphotransferase (ATP + H+ + Propionate --> ADP + | | |
| | | | CAETHG_3510 | Propionyl phosphate), 2,6-Diaminoheptanedioate: 2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 14 | Yield- | 3 | CAETHG_2932, | Alpha-acetolactate decarboxylase (ALCTT --> CO2 + (R)-Acetoin), | 0 | 0.040385 |
| | LMOMA | | CAETHG_3358, | Phosphate transacetylase (Phosphate + Acetyl-CoA + H+ <=> CoA + | | |
| | | | CAETHG_3510 | Acetylphosphate), 2,6-Diaminoheptanedioate: 2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 15 | Yield- | 3 | CAETHG_1270, | AMP: pyrophosphate phosphoribosyltransferase (PPi + AMP <=> | 0 | 0.035525 |
| | LMOMA | | CAETHG_2932, | PRPP + Adenine), Alpha-acetolactate decarboxylase (ALCTT --> | | |
| | | | CAETHG_3358 | CO2 + (R)-Acetoin), Phosphate transacetylase (Phosphate + | | |
| | | | | Acetyl-CoA + H+ <=> CoA + Acetylphosphate) | | |
| 16 | Yield- | 3 | CAETHG_0160, | N-Ribosylnicotinamide: orthophosphate ribosyltransferase | 0 | 0.035525 |
| | LMOMA | | CAETHG_2932, | (Phosphate + N-Ribosylnicotinamide <=> Nicotinamide + Ribose 1- | | |
| | | | CAETHG_3358 | phosphate), Alpha-acetolactate decarboxylase (ALCTT --> CO2 + | | |
| | | | | (R)-Acetoin), Phosphate transacetylase (Phosphate + Acetyl-CoA + | | |
| | | | | H+ <=> CoA + Acetylphosphate) | | |
| 17 | Yield- | 3 | CAETHG_2932, | Alpha-acetolactate decarboxylase (ALCTT --> CO2 + (R)-Acetoin), | 0 | 0.035525 |
| | LMOMA | | CAETHG_3358, | Phosphate transacetylase (Phosphate + Acetyl-CoA + H+ <=> CoA + | | |
| | | | CAETHG_3924 | Acetylphosphate), D-Ribose 1,5-phosphomutase (Ribose 1- | | |
| | | | | phosphate <=> ribose-5-phosphate) | | |
| 18 | Yield- | 3 | CAETHG_2475, | dGTP triphosphohydrolase (H2O + dGTP --> H+ + Deoxyguanosine + | 0 | 0.03288 |
| | LMOMA | | CAETHG_2932, | Triphosphate), Alpha-acetolactate decarboxylase (ALCTT --> CO2 + | | |
| | | | CAETHG_3358 | (R)-Acetoin), Phosphate transacetylase (Phosphate + Acetyl-CoA + | | |
| | | | | H+ <=> CoA + Acetylphosphate) | | |
| 19 | Yield- | 3 | 0, | Acetyl-CoA: carbon-dioxide ligase (ADP-forming) (ATP + Acetyl- | 0 | 0.03003 |
| | LMOMA | | CAETHG_2932, | CoA + H2CO3 --> ADP + Phosphate + H+ + Malonyl-CoA), Alpha- | | |
| | | | CAETHG_3358 | acetolactate decarboxylase (ALCTT --> CO2 + (R)-Acetoin), | | |
| | | | | Phosphate transacetylase (Phosphate + Acetyl-CoA + H+ <=> CoA + | | |
| | | | | Acetylphosphate) | | |
| 20 | Yield- | 3 | CAETHG_2932, | Alpha-acetolactate decarboxylase (ALCTT --> CO2 + (R)-Acetoin), | 0 | 0.029935 |
| | LMOMA | | CAETHG_3358, | Phosphate transacetylase (Phosphate + Acetyl-CoA + H+ <=> CoA + | | |
| | | | CAETHG_0498 | Acetylphosphate), Cystathionine beta lyase (H2O + | | |
| | | | | Cystathionine -- > NH3 + Pyruvate + Homocysteine) | | |
| 21 | Yield- | 3 | CAETHG_0234, | 4,5-Dihydro-4-oxo-5-imidazolepropanoate hydro-lyase (4- | 0 | 0.029835 |
| | LMOMA | | CAETHG_2932, | Imidazolone-5-propanoate <=> H2O + Urocanate), Alpha- | | |
| | | | CAETHG_3358 | acetolactate decarboxylase (ALCTT --> CO2 + (R)-Acetoin), | | |
| | | | | Phosphate transacetylase (Phosphate + Acetyl-CoA + H+ <=> CoA + | | |
| | | | | Acetylphosphate) | | |
| 22 | Yield- | 3 | CAETHG_0233, | 4-imidazolone-5-propanoate amidohydrolase (H2O + 4- | 0 | 0.029835 |
| | LMOMA | | CAETHG_2932, | Imidazolone-5-propanoate --> N-Formimino-L-glutamate), Alpha- | | |
| | | | CAETHG_3358 | acetolactate decarboxylase (ALCTT --> CO2 + (R)-Acetoin), | | |
| | | | | Phosphate transacetylase (Phosphate + Acetyl-CoA + H+ <=> CoA + | | |
| | | | | Acetylphosphate) | | |
| 23 | Yield- | 3 | CAETHG_2932, | Alpha-acetolactate decarboxylase (ALCTT --> CO2 + (R)-Acetoin), | 0 | 0.02962 |
| | LMOMA | | CAETHG_3164, | UMP: pyrophosphate phosphoribosyltransferase (Uracil + PRPP --> | | |
| | | | CAETHG_3358 | PPi + UMP), Phosphate transacetylase (Phosphate + Acetyl-CoA + | | |
| | | | | H+ <=> CoA + Acetylphosphate) | | |
| 24 | Yield- | 3 | CAETHG_2932, | Alpha-acetolactate decarboxylase (ALCTT --> CO2 + (R)-Acetoin), | 0 | 0.02948 |
| | LMOMA | | CAETHG_3358, | Phosphate transacetylase (Phosphate + Acetyl-CoA + H+ <=> CoA + | | |
| | | | CAETHG_0909 | Acetylphosphate), Prephenate: NAD+ | | |
| | | | | oxidoreductase(decarboxylating) (NAD + Prephenate --> NADH + | | |
| | | | | CO2 + p-hydroxyphenylpyruvate) | | |
| 25 | Yield- | 2 | CAETHG_2932, | Alpha-acetolactate decarboxylase (ALCTT --> CO2 + (R)-Acetoin), | 0 | 0.029455 |
| | LMOMA | | CAETHG_3359 | ATP: acetate phosphotransferase (ATP + H+ + Propionate --> ADP + | | |
| | | | | Propionyl phosphate) | | |
| 26 | Yield- | 2 | CAETHG_2932, | Alpha-acetolactate decarboxylase (ALCTT --> CO2 + (R)-Acetoin), | 0 | 0.029455 |
| | LMOMA | | CAETHG_3358 | Phosphate transacetylase (Phosphate + Acetyl-CoA + H+ <=> CoA + | | |
| | | | | Acetylphosphate) | | |
| 27 | Yield- | 2 | CAETHG_3358, | Phosphate transacetylase (Phosphate + Acetyl-CoA + H+ <=> CoA + | 0 | 0.028035 |
| | LMOMA | | CAETHG_3510 | Acetylphosphate), 2,6-Diaminoheptanedioate: 2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 28 | Yield- | 2 | CAETHG_3358, | Phosphate transacetylase (Phosphate + Acetyl-CoA + H+ <=> CoA + | 0 | 0.027595 |
| | LMOMA | | CAETHG_0498 | Acetylphosphate), Cystathionine beta lyase (H2O + | | |
| | | | | Cystathionine -- > NH3 + Pyruvate + Homocysteine) | | |
| 29 | Yield- | 2 | CAETHG_3021, | L-Arginine iminohydrolase (H2O + L-Arginine <=> NH3 + Citrulline), | 0 | 0.027555 |
| | LMOMA | | CAETHG_3358 | Phosphate transacetylase (Phosphate + Acetyl-CoA + H+ <=> CoA + | | |
| | | | | Acetylphosphate) | | |
| 30 | Yield- | 1 | CAETHG_3358 | Phosphate transacetylase (Phosphate + Acetyl-CoA + H+ <=> CoA + | 0 | 0.02754 |
| | LMOMA | | | Acetylphosphate) | | |
| 31 | Yield- | 1 | CAETHG_3359 | ATP: acetate phosphotransferase (ATP + H+ + Propionate --> ADP + | 0 | 0.02754 |
| | LMOMA | | | Propionyl phosphate) | | |
| 32 | Yield- | 2 | CAETHG_0102, | | 0 | 0.021305 |
| | LMOMA | | CAETHG_0092 | | | |
| 33 | Yield- | 2 | CAETHG_0160, | N-Ribosylnicotinamide: orthophosphate ribosyltransferase | 0 | 0.02015 |
| | LMOMA | | CAETHG_2721 | (Phosphate + N-Ribosylnicotinamide <=> Nicotinamide + Ribose 1- | | |
| | | | | phosphate), Phosphoenolpyruvate carboxykinase (PPCK) (ATP + | | |
| | | | | Oxaloacetate + H+ --> ADP + CO2 + Phosphoenolpyruvate) | | |
| 34 | Yield- | 2 | CAETHG_0160, | N-Ribosylnicotinamide: orthophosphate ribosyltransferase | 0 | 0.020115 |
| | LMOMA | | CAETHG_3021 | (Phosphate + N-Ribosylnicotinamide <=> Nicotinamide + Ribose 1- | | |
| | | | | phosphate), L-Arginine iminohydrolase (H2O + L-Arginine <=> | | |
| | | | | NH3 + Citrulline) | | |
| 35 | Yield- | 1 | CAETHG_0160 | N-Ribosylnicotinamide: orthophosphate ribosyltransferase | 0 | 0.02011 |
| | LMOMA | | | (Phosphate + N-Ribosylnicotinamide <=> Nicotinamide + Ribose 1- | | |
| | | | | phosphate) | | |
| 36 | Yield- | 1 | CAETHG_1371 | Adenosine 5′-monophosphate phosphohydrolase (H2O + AMP <=> | 0 | 0.02011 |
| | LMOMA | | | Phosphate + H+ + Adenosine) | | |
| 37 | Yield- | 1 | CAETHG_1270 | AMP: pyrophosphate phosphoribosyltransferase (PPi + AMP <=> | 0 | 0.02011 |
| | LMOMA | | | PRPP + Adenine) | | |
| 38 | Yield- | 1 | CAETHG_3924 | D-Ribose 1,5-phosphomutase (Ribose 1-phosphate <=> ribose-5- | 0 | 0.02011 |
| | LMOMA | | | phosphate) | | |
| 39 | Yield- | 1 | CAETHG_0498 | Cystathionine beta lyase (H2O + Cystathionine --> NH3 + | 0 | 0.020055 |
| | LMOMA | | | Pyruvate + Homocysteine) | | |
| 40 | Yield- | 1 | CAETHG_2721 | Phosphoenolpyruvate carboxykinase (PPCK) (ATP + Oxaloacetate + | 0 | 0.020055 |
| | LMOMA | | | H+ --> ADP + CO2 + Phosphoenolpyruvate) | | |
| 41 | Yield- | 1 | CAETHG_2751 | Citramalate synthase (H2O + Pyruvate + Acetyl-CoA <=> CoA + | 0 | 0.02003 |
| | LMOMA | | | Citramalate) | | |
| 42 | Yield- | 1 | CAETHG_3021 | L-Arginine iminohydrolase (H2O + L-Arginine <=> NH3 + Citrulline) | 0 | 0.020005 |
| | LMOMA | | | | | |

### Example 15

This example describes disruptions for improved production of adipic acid in Wood-Ljungdahl microorganisms. Metabolic modeling experiments were performed as described in Example 1. Production of adipic acid in Wood-Ljungdahl microorganisms is described, e.g., in WO 2017/0066498. The following pathway was used to model adipic acid production herein: ATP+CoA+Succinate-->ADP+Phosphate+Succinyl-CoA; NAD+CoA+2-Oxoglutarate-->NADH+C02+Succinyl-CoA; Acetyl-CoA+Succinyl-CoA-->CoA+3-Oxo-adipyl-CoA; NADH+H++3-Oxo-adipyl-CoA-->NAD+3-Hydroxyadipyl-CoA; 3-Hydroxyadipyl-CoA-->2,3-Dehydroadipyl-CoA; NADH+H++2,3-Dehydroadipyl-CoA-->NAD+Adipyl-CoA; Phosphate+Adipyl-CoA-->CoA+Adipyl-P; ADP+Adipyl-P-->ATP+Adipate; Adipate-->Adipate_ext.

| | | # | | | FVA | |
|---|---|---|---|---|---|---|
| | | Disrupted | Disrupted | | minimum | Fitness |
| # | Technique | genes | genes | Disrupted reactions | yield | score |
| 1 | BPCY- | 1 | CAETHG_2024 | Glutamine synthetase (GOGAT) (ATP + NH3 + | 0.02858 | 0.003813 |
| | FBA | | | L-Glutamate --> ADP + Phosphate + L-Glutamine + H+) | | |
| 2 | Yield- | 3 | CAETHG_2024, | Glutamine synthetase (GOGAT) (ATP + NH3 + | 0.09198 | 0.057966 |
| | LMOMA | | CAETHG_2062, | L-Glutamate --> ADP + Phosphate + L-Glutamine + H+) | | |
| | | | CAETHG_2479 | | | |
| 3 | Yield- | 5 | CAETHG_2024, | Glutamine synthetase (GOGAT) (ATP + NH3 + L-Glutamate --> | 0.02786 | 0.028758 |
| | LMOMA | | CAETHG_2799, | ADP + Phosphate + L-Glutamine + H+), NADP- | | |
| | | | CAETHG_2909, | dependent electron-bifurcating +FeFe+-hydrogenase (Hyt) | | |
| | | | CAETHG_2932, | (NADP + Oxidizedferredoxin + 2.0 H2 <=> NADPH + 3.0 H+ + | | |
| | | | CAETHG_0909 | Reducedferredoxin), ATP: pyruvate,orthophosphate | | |
| | | | | phosphotransferase (ATP + Phosphate + Pyruvate + H+ --> | | |
| | | | | PPi + AMP + Phosphoenolpyruvate), Alpha-acetolactate | | |
| | | | | decarboxylase (ALCTT --> CO2 + (R)-Acetoin), | | |
| | | | | Prephenate: NAD+ oxidoreductase(decarboxylating) (NAD+ | | |
| | | | | Prephenate --> NADH + CO2 + p-hydroxyphenylpyruvate) | | |
| 4 | Yield- | 4 | CAETHG_2024, | Glutamine synthetase (GOGAT) (ATP + NH3 + L-Glutamate --> | 0.02787 | 0.028566 |
| | LMOMA | | CAETHG_2796, | ADP + Phosphate + L-Glutamine + H+), NADP- | | |
| | | | CAETHG_2909, | dependent electron-bifurcating +FeFe+-hydrogenase (Hyt) | | |
| | | | CAETHG_2932 | (NADP + Oxidizedferredoxin + 2.0 H2 <=> NADPH + 3.0 H+ + | | |
| | | | | Reducedferredoxin), ATP: pyruvate,orthophosphate | | |
| | | | | phosphotransferase (ATP + Phosphate + Pyruvate + H+ --> | | |
| | | | | PPi + AMP + Phosphoenolpyruvate), Alpha-acetolactate | | |
| | | | | decarboxylase (ALCTT --> CO2 + (R)-Acetoin) | | |
| 5 | Yield- | 4 | CAETHG_2024, | Glutamine synthetase (GOGAT) (ATP + NH3 + L-Glutamate --> | 0.02787 | 0.028566 |
| | LMOMA | | CAETHG_2795, | ADP + Phosphate + L-Glutamine + H+), NADP- | | |
| | | | CAETHG_2909, | dependent electron-bifurcating +FeFe+-hydrogenase (Hyt) | | |
| | | | CAETHG_2932 | (NADP + Oxidizedferredoxin + 2.0 H2 <=> NADPH + 3.0 H+ + | | |
| | | | | Reducedferredoxin), ATP: pyruvate,orthophosphate | | |
| | | | | phosphotransferase (ATP + Phosphate + Pyruvate + H+ --> | | |
| | | | | PPi + AMP + Phosphoenolpyruvate), Alpha-acetolactate | | |
| | | | | decarboxylase (ALCTT --> CO2 + (R)-Acetoin) | | |
| 6 | Yield- | 3 | CAETHG_2024, | Glutamine synthetase (GOGAT) (ATP + NH3 + L-Glutamate --> | 0.02787 | 0.028464 |
| | LMOMA | | CAETHG_2909, | ADP + Phosphate + L-Glutamine + H+), | | |
| | | | CAETHG_2932 | ATP: pyruvate,orthophosphate phosphotransferase (ATP + | | |
| | | | | Phosphate + Pyruvate + H+ --> PPi + AMP + | | |
| | | | | Phosphoenolpyruvate), Alpha-acetolactate decarboxylase | | |
| | | | | (ALCTT --> CO2 + (R)-Acetoin) | | |
| 7 | Yield- | 3 | CAETHG_2024, | Glutamine synthetase (GOGAT) (ATP + NH3 + L-Glutamate --> | 0.02858 | 0.028086 |
| | LMOMA | | CAETHG_2794, | ADP + Phosphate + L-Glutamine + H+), NADP- | | |
| | | | CAETHG_2932 | dependent electron-bifurcating +FeFe+-hydrogenase (Hyt) | | |
| | | | | (NADP + Oxidizedferredoxin + 2.0 H2 <=> NADPH + 3.0 H+ + | | |
| | | | | Reducedferredoxin), Alpha-acetolactate decarboxylase | | |
| | | | | (ALCTT --> CO2 + (R)-Acetoin) | | |
| 8 | Yield- | 3 | CAETHG_2024, | Glutamine synthetase (GOGAT) (ATP + NH3 + L-Glutamate --> | 0.02858 | 0.028086 |
| | LMOMA | | CAETHG_2795, | ADP + Phosphate + L-Glutamine + H+), NADP- | | |
| | | | CAETHG_2932 | dependent electron-bifurcating +FeFe+-hydrogenase (Hyt) | | |
| | | | | (NADP + Oxidizedferredoxin + 2.0 H2 <=> NADPH + 3.0 H+ + | | |
| | | | | Reducedferredoxin), Alpha-acetolactate decarboxylase | | |
| | | | | (ALCTT --> CO2 + (R)-Acetoin) | | |
| 9 | Yield- | 3 | CAETHG_2024, | Glutamine synthetase (GOGAT) (ATP + NH3 + L-Glutamate --> | 0.02858 | 0.028086 |
| | LMOMA | | CAETHG_2799, | ADP + Phosphate + L-Glutamine + H+), NADP- | | |
| | | | CAETHG_2932 | dependent electron-bifurcating +FeFe+-hydrogenase (Hyt) | | |
| | | | | (NADP + Oxidizedferredoxin + 2.0 H2 <=> NADPH + 3.0 H+ + | | |
| | | | | Reducedferredoxin), Alpha-acetolactate decarboxylase | | |
| | | | | (ALCTT --> CO2 + (R)-Acetoin) | | |
| 10 | Yield- | 3 | CAETHG_2024, | Glutamine synthetase (GOGAT) (ATP + NH3 + L-Glutamate --> | 0.02858 | 0.028086 |
| | LMOMA | | CAETHG_2796, | ADP + Phosphate + L-Glutamine + H+), NADP- | | |
| | | | CAETHG_2932 | dependent electron-bifurcating +FeFe+-hydrogenase (Hyt) | | |
| | | | | (NADP + Oxidizedferredoxin + 2.0 H2 <=> NADPH + 3.0 H+ + | | |
| | | | | Reducedferredoxin), Alpha-acetolactate decarboxylase | | |
| | | | | (ALCTT --> CO2 + (R)-Acetoin) | | |
| 11 | Yield- | 3 | CAETHG_2024, | Glutamine synthetase (GOGAT) (ATP + NH3 + L-Glutamate --> | 0.02858 | 0.028086 |
| | LMOMA | | CAETHG_2798, | ADP + Phosphate + L-Glutamine + H+), NADP- | | |
| | | | CAETHG_2932 | dependent electron-bifurcating +FeFe+-hydrogenase (Hyt) | | |
| | | | | (NADP + Oxidizedferredoxin + 2.0 H2 <=> NADPH + 3.0 H+ + | | |
| | | | | Reducedferredoxin), Alpha-acetolactate decarboxylase | | |
| | | | | (ALCTT --> CO2 + (R)-Acetoin) | | |
| 12 | Yield- | 3 | CAETHG_2024, | Glutamine synthetase (GOGAT) (ATP + NH3 + L-Glutamate --> | 0.02858 | 0.028086 |
| | LMOMA | | CAETHG_2797, | ADP + Phosphate + L-Glutamine + H+), NADP- | | |
| | | | CAETHG_2932 | dependent electron-bifurcating +FeFe+-hydrogenase (Hyt) | | |
| | | | | (NADP + Oxidizedferredoxin + 2.0 H2 <=> NADPH + 3.0 H+ + | | |
| | | | | Reducedferredoxin), Alpha-acetolactate decarboxylase | | |
| | | | | (ALCTT --> CO2 + (R)-Acetoin) | | |
| 13 | Yield- | 3 | CAETHG_2024, | Glutamine synthetase (GOGAT) (ATP + NH3 + L-Glutamate --> | 0.02854 | 0.028074 |
| | LMOMA | | CAETHG_2932, | ADP + Phosphate + L-Glutamine + H+), Alpha- | | |
| | | | CAETHG_3021 | acetolactate decarboxylase (ALCTT --> CO2 + (R)-Acetoin), | | |
| | | | | L-Arginine iminohydrolase (H2O + L-Arginine <=> NH3 + | | |
| | | | | Citrulline) | | |
| 14 | Yield- | 3 | 0, | Acetyl-CoA: carbon-dioxide ligase (ADP-forming) (ATP + 0.02858 | | 0.028032 |
| | LMOMA | | CAETHG_2024, | Acetyl-CoA + H2CO3 --> ADP + Phosphate + H+ + Malonyl- | | |
| | | | CAETHG_2932 | CoA), Glutamine synthetase (GOGAT) (ATP + NH3 + L- | | |
| | | | | Glutamate --> ADP + Phosphate + L-Glutamine + H+), | | |
| | | | | Alpha-acetolactate decarboxylase (ALCTT --> CO2 + (R)- | | |
| | | | | Acetoin) | | |
| 15 | Yield- | 2 | CAETHG_2024, | Glutamine synthetase (GOGAT) (ATP + NH3 + L-Glutamate --> | 0.02858 | 0.028002 |
| | LMOMA | | CAETHG_2932 | ADP + Phosphate + L-Glutamine + H+), Alpha- | | |
| | | | | acetolactate decarboxylase (ALCTT --> CO2 + (R)-Acetoin) | | |
| 16 | Yield- | 2 | CAETHG_2024, | Glutamine synthetase (GOGAT) (ATP + NH3 + L-Glutamate --> | 0.02858 | 0.027156 |
| | LMOMA | | CAETHG_2475 | ADP + Phosphate + L-Glutamine + H+), dGTP | | |
| | | | | triphosphohydrolase (H2O + dGTP --> H+ + | | |
| | | | | Deoxyguanosine + Triphosphate) | | |
| 17 | Yield- | 2 | CAETHG_2024, | Glutamine synthetase (GOGAT) (ATP + NH3 + L-Glutamate --> | 0.02858 | 0.027156 |
| | LMOMA | | CAETHG_3299 | ADP + Phosphate + L-Glutamine + H+), 2-Deoxy-D- | | |
| | | | | ribose-5-phosphate acetaldehyde-lyase (deoxyribose-5- | | |
| | | | | phosphate <=> Acetaldehyde + Glyceraldehyde3- | | |
| | | | | phosphate) | | |
| 18 | Yield- | 1 | CAETHG_2024 | Glutamine synthetase (GOGAT) (ATP + NH3 + L-Glutamate --> | 0.02858 | 0.02715 |
| | LMOMA | | | ADP + Phosphate + L-Glutamine + H+) | | |
| 19 | Yield- | 2 | CAETHG_2024, | Glutamine synthetase (GOGAT) (ATP + NH3 + L-Glutamate --> | 0.02858 | 0.027126 |
| | LMOMA | | CAETHG_3510 | ADP + Phosphate + L-Glutamine + H+), 2,6- | | |
| | | | | Diaminoheptanedioate: 2-oxoglutarate aminotransferase | | |
| | | | | (H2O + L-Glutamate + H+ + tetrahydrodipicolinate <-- 2- | | |
| | | | | Oxoglutarate + LL-2,6-Diaminopimelate) | | |
| 20 | Yield- | 2 | CAETHG_1371, | Adenosine 5′-monophosphate phosphohydrolase (H2O + | 0 | 0.020616 |
| | LMOMA | | CAETHG_3021 | AMP <=> Phosphate + H+ + Adenosine), L-Arginine | | |
| | | | | iminohydrolase (H2O + L-Arginine <=> NH3 + Citrulline) | | |
| 21 | Yield- | 1 | CAETHG_1371 | Adenosine 5′-monophosphate phosphohydrolase (H2O + | 0 | 0.020592 |
| | LMOMA | | | AMP <=> Phosphate + H+ + Adenosine) | | |
| 22 | Yield- | 1 | CAETHG_3924 | D-Ribose 1,5-phosphomutase (Ribose 1-phosphate <=> | 0 | 0.020592 |
| | LMOMA | | | ribose-5-phosphate) | | |
| 23 | Yield- | 1 | CAETHG_0160 | N-Ribosylnicotinamide: orthophosphate ribosyltransferase 0 | | 0.020592 |
| | LMOMA | | | (Phosphate + N-Ribosylnicotinamide <=> Nicotinamide + | | |
| | | | | Ribose 1-phosphate) | | |
| 24 | Yield- | 1 | CAETHG_1270 | AMP: pyrophosphate phosphoribosyltransferase (PPi + | 0 | 0.020592 |
| | LMOMA | | | AMP <=> PRPP + Adenine) | | |
| 25 | Yield- | 1 | CAETHG_3021 | L-Arginine iminohydrolase (H2O + L-Arginine <=> NH3 + | 0 | 0.020034 |
| | LMOMA | | | Citrulline) | | |

### Example 16

This example describes disruptions for improved production of 2-aminobenzoate in Wood-Ljungdahl microorganisms. Metabolic modeling experiments were performed as described in Example 1. Production of 2-aminobenzoate in Wood-Ljungdahl microorganisms is described, e.g., in WO 2016/191625. The following pathway was used to model 2-aminobenzoate production herein: NH3+Chorismate=>H₂O+Pyruvate+H++2-aminobenzoate.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | Yield- | 5 | 0, | Acetyl-CoA: carbon-dioxide ligase (ADP-forming) (ATP + Acetyl- | 0 | 0.025375 |
| | LMOMA | | CAETHG_3021, | CoA + H2CO3 --> ADP + Phosphate + H+ + Malonyl-CoA), L- | | |
| | | | CAETHG_3359, | Arginine iminohydrolase (H2O + L-Arginine <=> NH3 + Citrulline), | | |
| | | | CAETHG_3510, | ATP: acetate phosphotransferase (ATP + H+ + Propionate --> | | |
| | | | CAETHG_3924 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), D-Ribose 1,5-phosphomutase (Ribose 1- | | |
| | | | | phosphate <=> ribose-5-phosphate) | | |
| 2 | Yield- | 4 | 0, | Acetyl-CoA: carbon-dioxide ligase (ADP-forming) (ATP + Acetyl- | 0 | 0.025368 |
| | LMOMA | | CAETHG_3359, | CoA + H2CO3 --> ADP + Phosphate + H+ + Malonyl-CoA), | | |
| | | | CAETHG_3510, | ATP: acetate phosphotransferase (ATP + H+ + Propionate --> | | |
| | | | CAETHG_3924 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), D-Ribose 1,5-phosphomutase (Ribose 1- | | |
| | | | | phosphate <=> ribose-5-phosphate) | | |
| 3 | Yield- | 4 | 0, | Acetyl-CoA: carbon-dioxide ligase (ADP-forming) (ATP + Acetyl- | 0 | 0.025368 |
| | LMOMA | | CAETHG_1270, | CoA + H2CO3 --> ADP + Phosphate + H+ + Malonyl-CoA), | | |
| | | | CAETHG_3359, | AMP: pyrophosphate phosphoribosyltransferase (PPi + AMP <=> | | |
| | | | CAETHG_3510 | PRPP + Adenine), ATP: acetate phosphotransferase (ATP + H+ + | | |
| | | | | Propionate --> ADP + Propionyl phosphate), 2,6- | | |
| | | | | Diaminoheptanedioate: 2-oxoglutarate aminotransferase (H2O + | | |
| | | | | L-Glutamate + H+ + tetrahydrodipicolinate <-- 2-Oxoglutarate + | | |
| | | | | LL-2,6-Diaminopimelate) | | |
| 4 | Yield- | 4 | CAETHG_2753, | Isocitrate dehydrogenase (NAD + Isocitrate <=> NADH + CO2 + 2- | 0 | 0.025361 |
| | LMOMA | | CAETHG_3359, | Oxoglutarate + H+), ATP: acetate phosphotransferase (ATP + H+ + | | |
| | | | CAETHG_3510, | Propionate --> ADP + Propionyl phosphate), 2,6- | | |
| | | | CAETHG_0686 | Diaminoheptanedioate: 2-oxoglutarate aminotransferase (H2O + | | |
| | | | | L-Glutamate + H+ + tetrahydrodipicolinate <-- 2-Oxoglutarate + | | |
| | | | | LL-2,6-Diaminopimelate), L-Threonine acetaldehyde-lyase (L- | | |
| | | | | Threonine --> Glycine + Acetaldehyde) | | |
| 5 | Yield- | 5 | 0, | Acetyl-CoA: carbon-dioxide ligase (ADP-forming) (ATP + Acetyl- | 0 | 0.025305 |
| | LMOMA | | CAETHG_2932, | CoA + H2CO3 --> ADP + Phosphate + H+ + Malonyl-CoA), Alpha- | | |
| | | | CAETHG_3359, | acetolactate decarboxylase (ALCTT --> CO2 + (R)-Acetoin), | | |
| | | | CAETHG_3510, | ATP: acetate phosphotransferase (ATP + H+ + Propionate --> | | |
| | | | CAETHG_0498 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 6 | Yield- | 5 | CAETHG_2932, | Alpha-acetolactate decarboxylase (ALCTT --> CO2 + (R)-Acetoin), | 0 | 0.025277 |
| | LMOMA | | CAETHG_3359, | ATP: acetate phosphotransferase (ATP + H+ + Propionate --> | | |
| | | | CAETHG_3510, | ADP + Propionyl phosphate), 2,6-Diaminoheptanedioate: 2- | | |
| | | | CAETHG_3924, | oxoglutarate aminotransferase (H2O + L-Glutamate + H+ + | | |
| | | | CAETHG_0498 | tetrahydrodipicolinate <-- 2-Oxoglutarate + LL-2,6- | | |
| | | | | Diaminopimelate), D-Ribose 1,5-phosphomutase (Ribose 1- | | |
| | | | | phosphate <=> ribose-5-phosphate), Cystathionine beta lyase | | |
| | | | | (H2O + Cystathionine --> NH3 + Pyruvate + Homocysteine) | | |
| 7 | Yield- | 3 | 0, | Acetyl-CoA: carbon-dioxide ligase (ADP-forming) (ATP + Acetyl- | 0 | 0.025256 |
| | LMOMA | | CAETHG_3359, | CoA + H2CO3 --> ADP + Phosphate + H+ + Malonyl-CoA), | | |
| | | | CAETHG_3510 | ATP: acetate phosphotransferase (ATP + H+ + Propionate --> | | |
| | | | | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 8 | Yield- | 4 | CAETHG_0160, | N-Ribosylnicotinamide: orthophosphate ribosyltransferase | 0 | 0.025235 |
| | LMOMA | | CAETHG_2753, | (Phosphate + N-Ribosylnicotinamide <=> Nicotinamide + Ribose 1- | | |
| | | | CAETHG_3358, | phosphate), Isocitrate dehydrogenase (NAD + Isocitrate <=> | | |
| | | | CAETHG_3510 | NADH + CO2 + 2-Oxoglutarate + H+), Phosphate transacetylase | | |
| | | | | (Phosphate + Acetyl-CoA + H+ <=> CoA + Acetylphosphate), 2,6- | | |
| | | | | Diaminoheptanedioate: 2-oxoglutarate aminotransferase (H2O + | | |
| | | | | L-Glutamate + H+ + tetrahydrodipicolinate <-- 2-Oxoglutarate + | | |
| | | | | LL-2,6-Diaminopimelate) | | |
| 9 | Yield- | 3 | CAETHG_3359, | ATP: acetate phosphotransferase (ATP + H+ + Propionate --> | 0 | 0.025221 |
| | LMOMA | | CAETHG_3510, | ADP + Propionyl phosphate), 2,6-Diaminoheptanedioate: 2- | | |
| | | | CAETHG_3924 | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate), D-Ribose 1,5-phosphomutase (Ribose 1- | | |
| | | | | phosphate <=> ribose-5-phosphate) | | |
| 10 | Yield- | 3 | CAETHG_1270, | AMP: pyrophosphate phosphoribosyltransferase (PPi + AMP <=> | 0 | 0.025221 |
| | LMOMA | | CAETHG_3359, | PRPP + Adenine), ATP: acetate phosphotransferase (ATP + H+ + | | |
| | | | CAETHG_3510 | Propionate --> ADP + Propionyl phosphate), 2,6- | | |
| | | | | Diaminoheptanedioate: 2-oxoglutarate aminotransferase (H2O + | | |
| | | | | L-Glutamate + H+ + tetrahydrodipicolinate <-- 2-Oxoglutarate + | | |
| | | | | LL-2,6-Diaminopimelate) | | |
| 11 | Yield- | 3 | CAETHG_0160, | N-Ribosylnicotinamide: orthophosphate ribosyltransferase | 0 | 0.025221 |
| | LMOMA | | CAETHG_3358, | (Phosphate + N-Ribosylnicotinamide <=> Nicotinamide + Ribose 1- | | |
| | | | CAETHG_3510 | phosphate), Phosphate transacetylase (Phosphate + Acetyl-CoA + | | |
| | | | | H+ <=> CoA + Acetylphosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 12 | Yield- | 3 | CAETHG_0160, | N-Ribosylnicotinamide: orthophosphate ribosyltransferase | 0 | 0.025221 |
| | LMOMA | | CAETHG_3359, | (Phosphate + N-Ribosylnicotinamide <=> Nicotinamide + Ribose 1- | | |
| | | | CAETHG_3510 | phosphate), ATP: acetate phosphotransferase (ATP + H+ + | | |
| | | | | Propionate --> ADP + Propionyl phosphate), 2,6- | | |
| | | | | Diaminoheptanedioate: 2-oxoglutarate aminotransferase (H2O + | | |
| | | | | L-Glutamate + H+ + tetrahydrodipicolinate <-- 2-Oxoglutarate + | | |
| | | | | LL-2,6-Diaminopimelate) | | |
| 13 | Yield- | 4 | CAETHG_3021, | L-Arginine iminohydrolase (H2O + L-Arginine <=> NH3 + | 0 | 0.025088 |
| | LMOMA | | CAETHG_3293, | Citrulline), Cysteine desulfhydrase (H2O + L-Cysteine <=> NH3 + | | |
| | | | CAETHG_3359, | Pyruvate + H2S), ATP: acetate phosphotransferase (ATP + H+ + | | |
| | | | CAETHG_3510 | Propionate --> ADP + Propionyl phosphate), 2,6- | | |
| | | | | Diaminoheptanedioate: 2-oxoglutarate aminotransferase (H2O + | | |
| | | | | L-Glutamate + H+ + tetrahydrodipicolinate <-- 2-Oxoglutarate + | | |
| | | | | LL-2,6-Diaminopimelate) | | |
| 14 | Yield- | 3 | CAETHG_3293, | Cysteine desulfhydrase (H2O + L-Cysteine <=> NH3 + Pyruvate + | 0 | 0.025081 |
| | LMOMA | | CAETHG_3359, | H2S), ATP: acetate phosphotransferase (ATP + H+ + Propionate --> | | |
| | | | CAETHG_3510 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 15 | Yield- | 3 | CAETHG_3021, | L-Arginine iminohydrolase (H2O + L-Arginine <=> NH3 + | 0 | 0.025081 |
| | LMOMA | | CAETHG_3359, | Citrulline), ATP: acetate phosphotransferase (ATP + H+ + | | |
| | | | CAETHG_3510 | Propionate --> ADP + Propionyl phosphate), 2,6- | | |
| | | | | Diaminoheptanedioate: 2-oxoglutarate aminotransferase (H2O + | | |
| | | | | L-Glutamate + H+ + tetrahydrodipicolinate <-- 2-Oxoglutarate + | | |
| | | | | LL-2,6-Diaminopimelate) | | |
| 16 | Yield- | 2 | CAETHG_3359, | ATP: acetate phosphotransferase (ATP + H+ + Propionate --> | 0 | 0.025074 |
| | LMOMA | | CAETHG_3510 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 17 | Yield- | 2 | CAETHG_3358, | Phosphate transacetylase (Phosphate + Acetyl-CoA + H+ <=> | 0 | 0.025074 |
| | LMOMA | | CAETHG_3510 | CoA + Acetylphosphate), 2,6-Diaminoheptanedioate: 2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 18 | Yield- | 2 | CAETHG_3359, | ATP: acetate phosphotransferase (ATP + H+ + Propionate --> | 0 | 0.025067 |
| | LMOMA | | CAETHG_3510 | ADP + Propionyl phosphate), 2,6-Diaminoheptanedioate: 2- | | |
| | | | | oxoglutarate aminotransferase (H2O + L-Glutamate + H+ + | | |
| | | | | tetrahydrodipicolinate <-- 2-Oxoglutarate + LL-2,6- | | |
| | | | | Diaminopimelate) | | |
| 19 | Yield- | 3 | CAETHG_3021, | L-Arginine iminohydrolase (H2O + L-Arginine <=> NH3 + | 0 | 0.024752 |
| | LMOMA | | CAETHG_3359, | Citrulline), ATP: acetate phosphotransferase (ATP + H+ + | | |
| | | | CAETHG_0498 | Propionate --> ADP + Propionyl phosphate), Cystathionine beta | | |
| | | | | lyase (H2O + Cystathionine --> NH3 + Pyruvate + Homocysteine) | | |
| 20 | Yield- | 2 | CAETHG_3359, | ATP: acetate phosphotransferase (ATP + H+ + Propionate --> | 0 | 0.024745 |
| | LMOMA | | CAETHG_0498 | ADP + Propionyl phosphate), Cystathionine beta lyase (H2O + | | |
| | | | | Cystathionine --> NH3 + Pyruvate + Homocysteine) | | |
| 21 | Yield- | 2 | CAETHG_3358, | Phosphate transacetylase (Phosphate + Acetyl-CoA + H+ <=> | 0 | 0.024738 |
| | LMOMA | | CAETHG_0498 | CoA + Acetylphosphate), Cystathionine beta lyase (H2O + | | |
| | | | | Cystathionine --> NH3 + Pyruvate + Homocysteine) | | |
| 22 | Yield- | 2 | CAETHG_3021, | L-Arginine iminohydrolase (H2O + L-Arginine <=> NH3 + | 0 | 0.024724 |
| | LMOMA | | CAETHG_3359 | Citrulline), ATP: acetate phosphotransferase (ATP + H+ + | | |
| | | | | Propionate --> ADP + Propionyl phosphate) | | |
| 23 | Yield- | 1 | CAETHG_3358 | Phosphate transacetylase (Phosphate + Acetyl-CoA + H+ <=> | 0 | 0.024717 |
| | LMOMA | | | CoA + Acetylphosphate) | | |
| 24 | Yield- | 1 | CAETHG_3359 | ATP: acetate phosphotransferase (ATP + H+ + Propionate --> | 0 | 0.024717 |
| | LMOMA | | | ADP + Propionyl phosphate) | | |
| 25 | Yield- | 1 | CAETHG_3293 | Cysteine desulfhydrase (H2O + L-Cysteine <=> NH3 + Pyruvate + | 0 | 0.020384 |
| | LMOMA | | | H2S) | | |
| 26 | Yield- | 2 | CAETHG_0498, | Cystathionine beta lyase (H2O + Cystathionine --> NH3 + | 0 | 0.020307 |
| | LMOMA | | CAETHG_0686 | Pyruvate + Homocysteine), L-Threonine acetaldehyde-lyase | | |
| | | | | (L-Threonine -- > Glycine + Acetaldehyde) | | |
| 27 | Yield- | 2 | CAETHG_2751, | Citramalate synthase (H2O + Pyruvate + Acetyl-CoA <=> CoA + | 0 | 0.020167 |
| | LMOMA | | CAETHG_0498 | Citramalate), Cystathionine beta lyase (H2O + Cystathionine --> | | |
| | | | | NH3 + Pyruvate + Homocysteine) | | |
| 28 | Yield- | 2 | CAETHG_3021, | L-Arginine iminohydrolase (H2O + L-Arginine <=> NH3 + | 0 | 0.020132 |
| | LMOMA | | CAETHG_0498 | Citrulline), Cystathionine beta lyase (H2O + Cystathionine --> | | |
| | | | | NH3 + Pyruvate + Homocysteine) | | |
| 29 | Yield- | 1 | CAETHG_0498 | Cystathionine beta lyase (H2O + Cystathionine --> NH3 + | 0 | 0.020125 |
| | LMOMA | | | Pyruvate + Homocysteine) | | |
| 30 | Yield- | 2 | CAETHG_2751, | Citramalate synthase (H2O + Pyruvate + Acetyl-CoA <=> CoA + | 0 | 0.020076 |
| | LMOMA | | CAETHG_3021 | Citramalate), L-Arginine iminohydrolase (H2O + L-Arginine <=> | | |
| | | | | NH3 + Citrulline) | | |
| 31 | Yield- | 1 | CAETHG_2751 | Citramalate synthase (H2O + Pyruvate + Acetyl-CoA <=> CoA + | 0 | 0.020069 |
| | LMOMA | | | Citramalate) | | |
| 32 | Yield- | 1 | CAETHG_0160 | N-Ribosylnicotinamide: orthophosphate ribosyltransferase | 0 | 0.020013 |
| | LMOMA | | | (Phosphate + N-Ribosylnicotinamide <=> Nicotinamide + Ribose 1- | | |
| | | | | phosphate) | | |
| 33 | Yield- | 1 | CAETHG_1371 | Adenosine 5′-monophosphate phosphohydrolase (H2O + | 0 | 0.020013 |
| | LMOMA | | | AMP <=> Phosphate + H+ + Adenosine) | | |
| 34 | Yield- | 1 | CAETHG_1270 | AMP: pyrophosphate phosphoribosyltransferase (PPi + AMP <=> | 0 | 0.020013 |
| | LMOMA | | | PRPP + Adenine) | | |
| 35 | Yield- | 1 | CAETHG_3924 | D-Ribose 1,5-phosphomutase (Ribose 1-phosphate <=> ribose-5- | 0 | 0.020013 |
| | LMOMA | | | phosphate) | | |
| 36 | Yield- | 1 | CAETHG_0686 | L-Threonine acetaldehyde-lyase (L-Threonine --> Glycine + | 0 | 0.020006 |
| | LMOMA | | | Acetaldehyde) | | |

### Example 17

This example describes disruptions for improved production of salicylate in Wood-Ljungdahl microorganisms. Metabolic modeling experiments were performed as described in Example 1. Production of salicylate in Wood-Ljungdahl microorganisms is described, e.g., in WO 2016/191625. The following pathway was used to model salicylate production herein: Chorismate-->Isochorismate; Isochorismate-->Salicylate+Pyruvate.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | Yield- | 3 | 0, | Acetyl-CoA: carbon-dioxide ligase (ADP-forming) (ATP + Acetyl- | 0 | 0.020174 |
| | LMOMA | | CAETHG_3164, | CoA + H2CO3 --> ADP + Phosphate + H+ + Malonyl-CoA), | | |
| | | | CAETHG_0686 | UMP: pyrophosphate phosphoribosyltransferase (Uracil + PRPP --> | | |
| | | | | PPi + UMP), L-Threonine acetaldehyde-lyase (L-Threonine --> | | |
| | | | | Glycine + Acetaldehyde) | | |
| 2 | Yield- | 3 | CAETHG_3021, | L-Arginine iminohydrolase (H2O + L-Arginine <=> NH3 + | 0 | 0.020174 |
| | LMOMA | | CAETHG_3299, | Citrulline), 2-Deoxy-D-ribose-5-phosphate acetaldehyde-lyase | | |
| | | | CAETHG_0686 | (deoxyribose-5-phosphate <=> Acetaldehyde + Glyceraldehyde3- | | |
| | | | | phosphate), L-Threonine acetaldehyde-lyase (L-Threonine --> | | |
| | | | | Glycine + Acetaldehyde) | | |
| 3 | Yield- | 1 | CAETHG_0686 | L-Threonine acetaldehyde-lyase (L-Threonine --> Glycine + | 0 | 0.020167 |
| | LMOMA | | | Acetaldehyde) | | |

### Example 18

This example describes disruptions for improved production of chorismate in Wood-Ljungdahl microorganisms. Metabolic modeling experiments were performed as described in Example 1. Production of chorismate in Wood-Ljungdahl microorganisms is described, e.g., in WO 2016/191625. Chorismate is a native product of at least some Wood-Ljungdahl microorganisms. These same disruptions would be expected to similarly increase flux through dehydroshikimate, a metabolic node just upstream of chorismate.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | Yield- | 4 | CAETHG_1270, | AMP: pyrophosphate phosphoribosyltransferase (PPi + AMP <=> | 0 | 0.02128 |
| | LMOMA | | CAETHG_2107, | PRPP + Adenine), Potassium uptake (K+_ext <=> K+), L-Arginine | | |
| | | | CAETHG_3021, | iminohydrolase (H2O + L-Arginine <=> NH3 + Citrulline), | | |
| | | | CAETHG_0498 | Cystathionine beta lyase (H2O + Cystathionine --> NH3 + | | |
| | | | | Pyruvate + Homocysteine) | | |
| 2 | Yield- | 4 | CAETHG_0160, | N-Ribosylnicotinamide: orthophosphate ribosyltransferase | 0 | 0.02128 |
| | LMOMA | | CAETHG_3021, | (Phosphate + N-Ribosylnicotinamide <=> Nicotinamide + Ribose 1- | | |
| | | | CAETHG_3510, | phosphate), L-Arginine iminohydrolase (H2O + L-Arginine <=> | | |
| | | | CAETHG_0498 | NH3 + Citrulline), 2,6-Diaminoheptanedioate: 2-oxoglutarate | | |
| | | | | aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 3 | Yield- | 4 | CAETHG_0160, | N-Ribosylnicotinamide: orthophosphate ribosyltransferase | 0 | 0.02128 |
| | LMOMA | | CAETHG_2107, | (Phosphate + N-Ribosylnicotinamide <=> Nicotinamide + Ribose 1- | | |
| | | | CAETHG_3021, | phosphate), Potassium uptake (K+_ext <=> K+), L-Arginine | | |
| | | | CAETHG_0498 | iminohydrolase (H2O + L-Arginine <=> NH3 + Citrulline), | | |
| | | | | Cystathionine beta lyase (H2O + Cystathionine --> NH3 + | | |
| | | | | Pyruvate + Homocysteine) | | |
| 4 | Yield- | 4 | CAETHG_3021, | L-Arginine iminohydrolase (H2O + L-Arginine <=> NH3 + | 0 | 0.02128 |
| | LMOMA | | CAETHG_3510, | Citrulline), 2,6-Diaminoheptanedioate: 2-oxoglutarate | | |
| | | | CAETHG_3924, | aminotransferase (H2O + L-Glutamate + H+ + | | |
| | | | CAETHG_0498 | tetrahydrodipicolinate <-- 2-Oxoglutarate + LL-2,6- | | |
| | | | | Diaminopimelate), D-Ribose 1,5-phosphomutase (Ribose 1- | | |
| | | | | phosphate <=> ribose-5-phosphate), Cystathionine beta lyase | | |
| | | | | (H2O + Cystathionine --> NH3 + Pyruvate + Homocysteine) | | |
| 5 | Yield- | 3 | CAETHG_1270, | AMP: pyrophosphate phosphoribosyltransferase (PPi + AMP <=> | 0 | 0.02127 |
| | LMOMA | | CAETHG_3021, | PRPP + Adenine), L-Arginine iminohydrolase (H2O + | | |
| | | | CAETHG_0498 | L-Arginine <=> NH3 + Citrulline), Cystathionine beta lyase (H2O + | | |
| | | | | Cystathionine --> NH3 + Pyruvate + Homocysteine) | | |
| 6 | Yield- | 3 | CAETHG_0160, | N-Ribosylnicotinamide: orthophosphate ribosyltransferase | 0 | 0.02127 |
| | LMOMA | | CAETHG_3021, | (Phosphate + N-Ribosylnicotinamide <=> Nicotinamide + Ribose 1- | | |
| | | | CAETHG_0498 | phosphate), L-Arginine iminohydrolase (H2O + L-Arginine <=> | | |
| | | | | NH3 + Citrulline), Cystathionine beta lyase (H2O + | | |
| | | | | Cystathionine -- > NH3 + Pyruvate + Homocysteine) | | |
| 7 | Yield- | 3 | CAETHG_1371, | Adenosine 5′-monophosphate phosphohydrolase (H2O + AMP <=> | 0 | 0.02127 |
| | LMOMA | | CAETHG_3021, | Phosphate + H+ + Adenosine), L-Arginine iminohydrolase | | |
| | | | CAETHG_0498 | (H2O + L-Arginine <=> NH3 + Citrulline), Cystathionine beta lyase | | |
| | | | | (H2O + Cystathionine --> NH3 + Pyruvate + Homocysteine) | | |
| 8 | Yield- | 3 | CAETHG_3021, | L-Arginine iminohydrolase (H2O + L-Arginine <=> NH3 + | 0 | 0.02127 |
| | LMOMA | | CAETHG_3924, | Citrulline), D-Ribose 1,5-phosphomutase (Ribose 1-phosphate <=> | | |
| | | | CAETHG_0498 | ribose-5-phosphate), Cystathionine beta lyase (H2O + | | |
| | | | | Cystathionine --> NH3 + Pyruvate + Homocysteine) | | |
| 9 | Yield- | 3 | CAETHG_0160, | N-Ribosylnicotinamide: orthophosphate ribosyltransferase | 0 | 0.0211 |
| | LMOMA | | CAETHG_2107, | (Phosphate + N-Ribosylnicotinamide <=> Nicotinamide + Ribose 1- | | |
| | | | CAETHG_0498 | phosphate), Potassium uptake (K+_ext <=> K+), Cystathionine | | |
| | | | | beta lyase (H2O + Cystathionine --> NH3 + Pyruvate + | | |
| | | | | Homocysteine) | | |
| 10 | Yield- | 3 | CAETHG_1270, | AMP: pyrophosphate phosphoribosyltransferase (PPi + AMP <=> | 0 | 0.0211 |
| | LMOMA | | CAETHG_3510, | PRPP + Adenine), 2,6-Diaminoheptanedioate: 2-oxoglutarate | | |
| | | | CAETHG_0498 | aminotransferase (H2O + L-Glutamate + H+ + | | |
| | | | | tetrahydrodipicolinate <-- 2-Oxoglutarate + LL-2,6- | | |
| | | | | Diaminopimelate), Cystathionine beta lyase (H2O + | | |
| | | | | Cystathionine --> NH3 + Pyruvate + Homocysteine) | | |
| 11 | Yield- | 3 | CAETHG_1270, | AMP: pyrophosphate phosphoribosyltransferase (PPi + AMP <=> | 0 | 0.0211 |
| | LMOMA | | CAETHG_2107, | PRPP + Adenine), Potassium uptake (K+_ext <=> K+), | | |
| | | | CAETHG_0498 | Cystathionine beta lyase (H2O + Cystathionine --> NH3 + | | |
| | | | | Pyruvate + Homocysteine) | | |
| 12 | Yield- | 2 | CAETHG_3924, | D-Ribose 1,5-phosphomutase (Ribose 1-phosphate <=> ribose-5- | 0 | 0.02109 |
| | LMOMA | | CAETHG_0498 | phosphate), Cystathionine beta lyase (H2O + Cystathionine --> | | |
| | | | | NH3 + Pyruvate + Homocysteine) | | |
| 13 | Yield- | 2 | CAETHG_1270, | AMP: pyrophosphate phosphoribosyltransferase (PPi + AMP <=> | 0 | 0.02109 |
| | LMOMA | | CAETHG_0498 | PRPP + Adenine), Cystathionine beta lyase (H2O + | | |
| | | | | Cystathionine -- > NH3 + Pyruvate + Homocysteine) | | |
| 14 | Yield- | 2 | CAETHG_1371, | Adenosine 5′-monophosphate phosphohydrolase (H2O + AMP <=> | 0 | 0.02109 |
| | LMOMA | | CAETHG_0498 | Phosphate + H+ + Adenosine), Cystathionine beta lyase (H2O + | | |
| | | | | Cystathionine --> NH3 + Pyruvate + Homocysteine) | | |
| 15 | Yield- | 2 | CAETHG_0160, | N-Ribosylnicotinamide: orthophosphate ribosyltransferase | 0 | 0.02109 |
| | LMOMA | | CAETHG_0498 | (Phosphate + N-Ribosylnicotinamide <=> Nicotinamide + Ribose 1- | | |
| | | | | phosphate), Cystathionine beta lyase (H2O + Cystathionine --> | | |
| | | | | NH3 + Pyruvate + Homocysteine) | | |
| 16 | Yield- | 2 | CAETHG_3021, | L-Arginine iminohydrolase (H2O + L-Arginine <=> NH3 + | 0 | 0.02065 |
| | LMOMA | | CAETHG_3924 | Citrulline), D-Ribose 1,5-phosphomutase (Ribose 1-phosphate <=> | | |
| | | | | ribose-5-phosphate) | | |
| 17 | Yield- | 2 | CAETHG_0248, | L-lactate reversible transport via proton symport (H+_ext + L- | 0 | 0.02064 |
| | LMOMA | | CAETHG_3924 | Lactate_ext <=> H+ + L-Lactate), D-Ribose 1,5-phosphomutase | | |
| | | | | (Ribose 1-phosphate <=> ribose-5-phosphate) | | |
| 18 | Yield- | 1 | CAETHG_1371 | Adenosine 5′-monophosphate phosphohydrolase (H2O + AMP <=> | 0 | 0.02062 |
| | LMOMA | | | Phosphate + H+ + Adenosine) | | |
| 19 | Yield- | 1 | CAETHG_3924 | D-Ribose 1,5-phosphomutase (Ribose 1-phosphate <=> ribose-5- | 0 | 0.02062 |
| | LMOMA | | | phosphate) | | |
| 20 | Yield- | 1 | CAETHG_0160 | N-Ribosylnicotinamide: orthophosphate ribosyltransferase | 0 | 0.02062 |
| | LMOMA | | | (Phosphate + N-Ribosylnicotinamide <=> Nicotinamide + Ribose 1- | | |
| | | | | phosphate) | | |
| 21 | Yield- | 1 | CAETHG_1270 | AMP: pyrophosphate phosphoribosyltransferase (PPi + AMP <=> | 0 | 0.02062 |
| | LMOMA | | | PRPP + Adenine) | | |
| 22 | Yield- | 1 | CAETHG_3021 | L-Arginine iminohydrolase (H2O + L-Arginine <=> | 0 | 0.02003 |
| | LMOMA | | | NH3 + Citrulline) | | |

### Example 19

This example describes disruptions for improved production of famesene in Wood-Ljungdahl microorganisms. Metabolic modeling experiments were performed as described in Example 1. Production of famesene in Wood-Ljungdahl microorganisms is described, e.g., in WO 2013/180584. The following pathway was used to model famesene production herein: 2.0 Acetyl-CoA-->CoA+Acetoacetyl-CoA; H₂O+Acetyl-CoA+Acetoacetyl-CoA-->CoA+(S)-3-Hydroxy-3-methylglutaryl-CoA; 2.0 NADH+2.0H++(S)-3-Hydroxy-3-methylglutaryl-CoA-->2.0 NAD+CoA+(R)-Mevalonate; ATP+(R)-Mevalonate-->ADP+(R)-5-Phosphomevalonate; ATP+(R)-5-Diphosphomevalonate-->ADP+Phosphate+CO2+Isopentenyldiphosphate; ATP+(R)-5-Diphosphomevalonate-->ADP+Phosphate+CO2+Isopentenyldiphosphate; Isopentenyldiphosphate-->DMAPP; 2.0 Isopentenyldiphosphate+DMAPP-->PPi+trans,trans-famesyl-diphosphate; trans,trans-famesyl-diphosphate-->Famesene+PPi; Famesene-->Famesene_ext.

| | | # | | | FVA | |
|---|---|---|---|---|---|---|
| | | Disrupted | | | minimum | Fitness |
| # | Technique | genes | Disrupted genes | Disrupted reactions | yield | score |
| 1 | Yield- | 5 | CAETHG_2062, | Cysteine desulfhydrase (H2O + L-Cysteine <=> NH3 + Pyruvate + | 0 | 0.023775 |
| | LMOMA | | CAETHG_2479, | H2S), ATP: acetate phosphotransferase (ATP + H+ + Propionate --> | | |
| | | | CAETHG_3293, | ADP + Propionyl phosphate), 2,6-Diaminoheptanedioate: 2- | | |
| | | | CAETHG_3359, | oxoglutarate aminotransferase (H2O + L-Glutamate + H+ + | | |
| | | | CAETHG_3510 | tetrahydrodipicolinate <-- 2-Oxoglutarate + LL-2,6- | | |
| | | | | Diaminopimelate) | | |
| 2 | Yield- | 5 | CAETHG_2062, | ATP: acetate phosphotransferase (ATP + H+ + Propionate --> | 0 | 0.02376 |
| | LMOMA | | CAETHG_2479, | ADP + Propionyl phosphate), 2,6-Diaminoheptanedioate: 2- | | |
| | | | CAETHG_3359, | oxoglutarate aminotransferase (H2O + L-Glutamate + H+ + | | |
| | | | CAETHG_3510, | tetrahydrodipicolinate <-- 2-Oxoglutarate + LL-2,6- | | |
| | | | CAETHG_0686 | Diaminopimelate), L-Threonine acetaldehyde-lyase | | |
| | | | | (L-Threonine --> Glycine + Acetaldehyde) | | |
| 3 | Yield- | 6 | CAETHG_2062, | Citramalate synthase (H2O + Pyruvate + Acetyl-CoA <=> CoA + | 0 | 0.02376 |
| | LMOMA | | CAETHG_2479, | Citramalate), ATP: pyruvate,orthophosphate phosphotransferase | | |
| | | | CAETHG_2751, | (ATP + Phosphate + Pyruvate + H+ --> PPi + AMP + | | |
| | | | CAETHG_2909, | Phosphoenolpyruvate), ATP: acetate phosphotransferase (ATP + | | |
| | | | CAETHG_3359, | H+ + Propionate --> ADP + Propionyl phosphate), 2,6- | | |
| | | | CAETHG_3510 | Diaminoheptanedioate: 2-oxoglutarate aminotransferase (H2O + | | |
| | | | | L-Glutamate + H+ + tetrahydrodipicolinate <-- 2-Oxoglutarate + | | |
| | | | | LL-2,6-Diaminopimelate) | | |
| 4 | Yield- | 5 | CAETHG_2062, | Alpha-acetolactate decarboxylase (ALCTT --> CO2 + (R)-Acetoin), | 0 | 0.02376 |
| | LMOMA | | CAETHG_2479, | ATP: acetate phosphotransferase (ATP + H+ + Propionate --> | | |
| | | | CAETHG_2932, | ADP+ Propionyl phosphate), 2,6-Diaminoheptanedioate: 2- | | |
| | | | CAETHG_3359, | oxoglutarate aminotransferase (H2O + L-Glutamate + H+ + | | |
| | | | CAETHG_3510 | tetrahydrodipicolinate <-- 2-Oxoglutarate + LL-2,6- | | |
| | | | | Diaminopimelate) | | |
| 5 | Yield- | 4 | CAETHG_2062, | ATP: acetate phosphotransferase (ATP + H+ + Propionate --> | 0 | 0.023745 |
| | LMOMA | | CAETHG_2479, | ADP + Propionyl phosphate), 2,6-Diaminoheptanedioate: 2- | | |
| | | | CAETHG_3359, | oxoglutarate aminotransferase (H2O + L-Glutamate + H+ + | | |
| | | | CAETHG_3510 | tetrahydrodipicolinate <-- 2-Oxoglutarate + LL-2,6- | | |
| | | | | Diaminopimelate) | | |
| 6 | Yield- | 4 | CAETHG_2062, | ATP: acetate phosphotransferase (ATP + H+ + Propionate --> | 0 | 0.02373 |
| | LMOMA | | CAETHG_2479, | ADP + Propionyl phosphate), 2,6-Diaminoheptanedioate: 2- | | |
| | | | CAETHG_3359, | oxoglutarate aminotransferase (H2O + L-Glutamate + H+ + | | |
| | | | CAETHG_3510 | tetrahydrodipicolinate <-- 2-Oxoglutarate + LL-2,6- | | |
| | | | | Diaminopimelate) | | |
| 7 | Yield- | 5 | CAETHG_2062, | Alpha-acetolactate decarboxylase (ALCTT --> CO2 + (R)-Acetoin), | 0 | 0.02364 |
| | LMOMA | | CAETHG_2479, | ATP: acetate phosphotransferase (ATP + H+ + Propionate --> | | |
| | | | CAETHG_2932, | ADP + Propionyl phosphate), L-Threonine acetaldehyde-lyase (L- | | |
| | | | CAETHG_3359, | Threonine --> Glycine + Acetaldehyde) | | |
| | | | CAETHG_0686 | | | |
| 8 | Yield- | 4 | 0, | Acetyl-CoA: carbon-dioxide ligase (ADP-forming) (ATP + Acetyl- | 0 | 0.0234 |
| | LMOMA | | CAETHG_1270, | CoA + H2CO3 --> ADP + Phosphate + H+ + Malonyl-CoA), | | |
| | | | CAETHG_3359, | AMP: pyrophosphate phosphoribosyltransferase (PPi + AMP <=> | | |
| | | | CAETHG_0498 | PRPP + Adenine), ATP: acetate phosphotransferase (ATP + H+ + | | |
| | | | | Propionate --> ADP + Propionyl phosphate), Cystathionine beta | | |
| | | | | lyase (H2O + Cystathionine --> NH3 + Pyruvate + Homocysteine) | | |
| 9 | Yield- | 3 | 0, | Acetyl-CoA: carbon-dioxide ligase (ADP-forming) (ATP + Acetyl- | 0 | 0.023385 |
| | LMOMA | | CAETHG_1270, | CoA + H2CO3 --> ADP + Phosphate + H+ + Malonyl-CoA), | | |
| | | | CAETHG_3359 | AMP: pyrophosphate phosphoribosyltransferase (PPi + AMP <=> | | |
| | | | | PRPP + Adenine), ATP: acetate phosphotransferase (ATP + H+ + | | |
| | | | | Propionate --> ADP + Propionyl phosphate) | | |
| 10 | Yield- | 3 | 0, | Acetyl-CoA: carbon-dioxide ligase (ADP-forming) (ATP + Acetyl- | 0 | 0.023325 |
| | LMOMA | | CAETHG_3359, | CoA + H2CO3 --> ADP + Phosphate + H+ + Malonyl-CoA), | | |
| | | | CAETHG_0498 | ATP: acetate phosphotransferase (ATP + H+ + Propionate --> | | |
| | | | | ADP + Propionyl phosphate), Cystathionine beta lyase (H2O + | | |
| | | | | Cystathionine --> NH3 + Pyruvate + Homocysteine) | | |
| 11 | Yield- | 3 | CAETHG_1270, | AMP: pyrophosphate phosphoribosyltransferase (PPi + AMP <=> | 0 | 0.02331 |
| | LMOMA | | CAETHG_3359, | PRPP + Adenine), ATP: acetate phosphotransferase (ATP + H+ + | | |
| | | | CAETHG_0498 | Propionate --> ADP + Propionyl phosphate), Cystathionine beta | | |
| | | | | lyase (H2O + Cystathionine --> NH3 + Pyruvate + Homocysteine) | | |
| 12 | Yield- | 2 | CAETHG_1371, | Adenosine 5′-monophosphate phosphohydrolase (H2O + AMP <=> | 0 | 0.023295 |
| | LMOMA | | CAETHG_3358 | Phosphate + H+ + Adenosine), Phosphate transacetylase | | |
| | | | | (Phosphate + Acetyl-CoA + H+ <=> CoA + Acetylphosphate) | | |
| 13 | Yield- | 2 | CAETHG_1371, | Adenosine 5′-monophosphate phosphohydrolase (H2O + AMP <=> | 0 | 0.023295 |
| | LMOMA | | CAETHG_3359 | Phosphate + H+ + Adenosine), ATP: acetate | | |
| | | | | phosphotransferase (ATP + H+ + Propionate --> ADP + Propionyl | | |
| | | | | phosphate) | | |
| 14 | Yield- | 2 | CAETHG_1270, | AMP: pyrophosphate phosphoribosyltransferase (PPi + AMP <=> | 0 | 0.023295 |
| | LMOMA | | CAETHG_3359 | PRPP + Adenine), ATP: acetate phosphotransferase (ATP + H+ + | | |
| | | | | Propionate --> ADP + Propionyl phosphate) | | |
| 15 | Yield- | 2 | CAETHG_0160, | N-Ribosylnicotinamide: orthophosphate ribosyltransferase | 0 | 0.023295 |
| | LMOMA | | CAETHG_3359 | (Phosphate + N-Ribosylnicotinamide <=> Nicotinamide + Ribose 1- | | |
| | | | | phosphate), ATP: acetate phosphotransferase (ATP + H+ + | | |
| | | | | Propionate --> ADP + Propionyl phosphate) | | |
| 16 | Yield- | 2 | CAETHG_3359, | ATP: acetate phosphotransferase (ATP + H+ + Propionate --> ADP + | 0 | 0.023295 |
| | LMOMA | | CAETHG_3924 | Propionyl phosphate), D-Ribose 1,5-phosphomutase (Ribose 1- | | |
| | | | | phosphate <=> ribose-5-phosphate) | | |
| 17 | Yield- | 2 | CAETHG_1270, | AMP: pyrophosphate phosphoribosyltransferase (PPi + AMP <=> | 0 | 0.023295 |
| | LMOMA | | CAETHG_3358 | PRPP + Adenine), Phosphate transacetylase (Phosphate + Acetyl- | | |
| | | | | CoA + H+ <=> CoA + Acetylphosphate) | | |
| 18 | Yield- | 2 | CAETHG_3359, | ATP: acetate phosphotransferase (ATP + H+ + Propionate --> | 0 | 0.02268 |
| | LMOMA | | CAETHG_0498 | ADP + Propionyl phosphate), Cystathionine beta lyase (H2O + | | |
| | | | | Cystathionine --> NH3 + Pyruvate + Homocysteine) | | |
| 19 | Yield- | 2 | CAETHG_3358, | Phosphate transacetylase (Phosphate + Acetyl-CoA + H+ <=> | 0 | 0.02268 |
| | LMOMA | | CAETHG_0498 | CoA + Acetylphosphate), Cystathionine beta lyase (H2O + | | |
| | | | | Cystathionine --> NH3 + Pyruvate + Homocysteine) | | |
| 20 | Yield- | 2 | 0, | Acetyl-CoA: carbon-dioxide ligase (ADP-forming) (ATP + Acetyl- | 0 | 0.022665 |
| | LMOMA | | CAETHG_3359 | CoA + H2CO3 --> ADP + Phosphate + H+ + Malonyl-CoA), | | |
| | | | | ATP: acetate phosphotransferase (ATP + H+ + Propionate --> | | |
| | | | | ADP + Propionyl phosphate) | | |
| 21 | Yield- | 2 | CAETHG_3359, | ATP: acetate phosphotransferase (ATP + H+ + Propionate --> | 0 | 0.022665 |
| | LMOMA | | CAETHG_0909 | ADP + Propionyl phosphate), Prephenate: NAD+ | | |
| | | | | oxidoreductase(decarboxylating) (NAD + Prephenate --> NADH + | | |
| | | | | CO2 + p-hydroxyphenylpyruvate) | | |
| 22 | Yield- | 2 | CAETHG_3359, | ATP: acetate phosphotransferase (ATP + H+ + Propionate --> | 0 | 0.022605 |
| | LMOMA | | CAETHG_0686 | ADP + Propionyl phosphate), L-Threonine acetaldehyde-lyase (L- | | |
| | | | | Threonine --> Glycine + Acetaldehyde) | | |
| 23 | Yield- | 2 | CAETHG_2932, | Alpha-acetolactate decarboxylase (ALCTT --> CO2 + (R)-Acetoin), | 0 | 0.02235 |
| | LMOMA | | CAETHG_3359 | ATP: acetate phosphotransferase (ATP + H+ + Propionate --> | | |
| | | | | ADP + Propionyl phosphate) | | |
| 24 | Yield- | 2 | CAETHG_3021, | L-Arginine iminohydrolase (H2O + L-Arginine <=> NH3 + | 0 | 0.022305 |
| | LMOMA | | CAETHG_3359 | Citrulline), ATP: acetate phosphotransferase (ATP + H+ + | | |
| | | | | Propionate --> ADP + Propionyl phosphate) | | |
| 25 | Yield- | 1 | CAETHG_3358 | Phosphate transacetylase (Phosphate + Acetyl-CoA + H+ <=> | 0 | 0.021945 |
| | LMOMA | | | CoA + Acetylphosphate) | | |
| 26 | Yield- | 1 | CAETHG_3359 | ATP: acetate phosphotransferase (ATP + H+ + Propionate --> | 0 | 0.021945 |
| | LMOMA | | | ADP + Propionyl phosphate) | | |
| 27 | Yield- | 2 | CAETHG_1270, | AMP: pyrophosphate phosphoribosyltransferase (PPi + AMP <=> | 0 | 0.020235 |
| | LMOMA | | CAETHG_0686 | PRPP + Adenine), L-Threonine acetaldehyde-lyase (L-Threonine --> | | |
| | | | | Glycine + Acetaldehyde) | | |
| 28 | Yield- | 2 | CAETHG_3293, | Cysteine desulfhydrase (H2O + L-Cysteine <=> NH3 + Pyruvate + | 0 | 0.020205 |
| | LMOMA | | CAETHG_0686 | H2S), L-Threonine acetaldehyde-lyase (L-Threonine --> Glycine + | | |
| | | | | Acetaldehyde) | | |
| 29 | Yield- | 2 | CAETHG_1371, | Adenosine 5′-monophosphate phosphohydrolase (H2O + AMP <=> | 0 | 0.02016 |
| | LMOMA | | CAETHG_2721 | Phosphate + H+ + Adenosine), Phosphoenolpyruvate | | |
| | | | | carboxykinase (PPCK) (ATP + Oxaloacetate + H+ --> ADP + CO2 + | | |
| | | | | Phosphoenolpyruvate) | | |
| 30 | Yield- | 2 | CAETHG_1371, | Adenosine 5′-monophosphate phosphohydrolase (H2O + AMP <=> | 0 | 0.020145 |
| | LMOMA | | CAETHG_0498 | Phosphate + H+ + Adenosine), Cystathionine beta lyase (H2O + | | |
| | | | | Cystathionine --> NH3 + Pyruvate + Homocysteine) | | |
| 31 | Yield- | 2 | CAETHG_2475, | dGTP triphosphohydrolase (H2O + dGTP --> H+ + Deoxyguanosine + | 0 | 0.02013 |
| | LMOMA | | CAETHG_0686 | Triphosphate), L-Threonine acetaldehyde-lyase (L-Threonine --> | | |
| | | | | Glycine + Acetaldehyde) | | |
| 32 | Yield- | 2 | CAETHG_1371, | Adenosine 5′-monophosphate phosphohydrolase (H2O + AMP <=> | 0 | 0.020115 |
| | LMOMA | | CAETHG_2751 | Phosphate + H+ + Adenosine), Citramalate synthase (H2O + | | |
| | | | | Pyruvate + Acetyl-CoA <=> CoA + Citramalate) | | |
| 33 | Yield- | 2 | 0, | Acetyl-CoA: carbon-dioxide ligase (ADP-forming) (ATP + Acetyl- | 0 | 0.0201 |
| | LMOMA | | CAETHG_1371 | CoA + H2CO3 --> ADP + Phosphate + H+ + Malonyl-CoA), | | |
| | | | | Adenosine 5′-monophosphate phosphohydrolase (H2O + AMP <=> | | |
| | | | | Phosphate + H+ + Adenosine) | | |
| 34 | Yield- | 2 | CAETHG_1371, | Adenosine 5′-monophosphate phosphohydrolase (H2O + AMP <=> | 0 | 0.0201 |
| | LMOMA | | CAETHG_3021 | Phosphate + H+ + Adenosine), L-Arginine iminohydrolase | | |
| | | | | (H2O + L-Arginine <=> NH3 + Citrulline) | | |
| 35 | Yield- | 2 | CAETHG_1371, | Adenosine 5′-monophosphate phosphohydrolase (H2O + AMP <=> | 0 | 0.0201 |
| | LMOMA | | CAETHG_0248 | Phosphate + H+ + Adenosine), L-lactate reversible transport | | |
| | | | | via proton symport (H+_ext + L-Lactate_ext <=> H+ + L-Lactate) | | |
| 36 | Yield- | 1 | CAETHG_0686 | L-Threonine acetaldehyde-lyase (L-Threonine --> Glycine + | 0 | 0.0201 |
| | LMOMA | | | Acetaldehyde) | | |
| 37 | Yield- | 1 | CAETHG_1270 | AMP: pyrophosphate phosphoribosyltransferase (PPi + AMP <=> | 0 | 0.020085 |
| | LMOMA | | | PRPP + Adenine) | | |
| 38 | Yield- | 1 | CAETHG_1371 | Adenosine 5′-monophosphate phosphohydrolase (H2O + AMP <=> | 0 | 0.020085 |
| | LMOMA | | | Phosphate + H+ + Adenosine) | | |
| 39 | Yield- | 1 | CAETHG_3924 | D-Ribose 1,5-phosphomutase (Ribose 1-phosphate <=> ribose-5- | 0 | 0.020085 |
| | LMOMA | | | phosphate) | | |
| 40 | Yield- | 1 | CAETHG_0160 | N-Ribosylnicotinamide: orthophosphate ribosyltransferase | 0 | 0.020085 |
| | LMOMA | | | (Phosphate + N-Ribosylnicotinamide <=> Nicotinamide + Ribose 1- | | |
| | | | | phosphate) | | |
| 41 | Yield- | 1 | CAETHG_2721 | Phosphoenolpyruvate carboxykinase (PPCK) (ATP + Oxaloacetate + | 0 | 0.02007 |
| | LMOMA | | | H+ --> ADP + CO2 + Phosphoenolpyruvate) | | |
| 42 | Yield- | 1 | CAETHG_0498 | Cystathionine beta lyase (H2O + Cystathionine --> NH3 + Pyruvate + | 0 | 0.020055 |
| | LMOMA | | | Homocysteine) | | |
| 43 | Yield- | 1 | CAETHG_2751 | Citramalate synthase (H2O + Pyruvate + Acetyl-CoA <=> CoA + | 0 | 0.020025 |
| | LMOMA | | | Citramalate) | | |
| 44 | Yield- | 2 | CAETHG_2909, | ATP: pyruvate,orthophosphate phosphotransferase (ATP + | 0 | 0.020025 |
| | LMOMA | | CAETHG_2932 | Phosphate + Pyruvate + H+ --> PPi + AMP + | | |
| | | | | Phosphoenolpyruvate), Alpha-acetolactate decarboxylase | | |
| | | | | (ALCTT --> CO2 + (R)-Acetoin) | | |
| 45 | Yield- | 1 | CAETHG_3021 | L-Arginine iminohydrolase (H2O + L-Arginine <=> NH3 + | 0 | 0.02001 |
| | LMOMA | | | Citrulline) | | |

### Example 20

This example describes gene disruption targets common across different product pathways. Optimizations were run using an evolutionary algorithm on 444 pathways. Each strain design was scored based on the achieved yield (non-growth coupled designs) and biomass-product coupled yield (growth coupled designs). Each gene was ranked based on how often it appeared in strain designs. 19 genes were commonly identified for disruption in optimized strains.

| | | Growth | Non-growth | Total |
|---|---|---|---|---|
| # | Gene | coupling score | coupling score | score |
| 1 | CAETHG_3359 | 6.039281 | 14.18996 | 20.22924 |
| 2 | CAETHG_2932 | 0 | 16.79651 | 16.79651 |
| 3 | CAETHG_3293 | 2.508019 | 13.12476 | 15.63278 |
| 4 | CAETHG_3510 | 0 | 12.77112 | 12.77112 |
| 5 | CAETHG_3924 | 0 | 11.15045 | 11.15045 |
| 6 | CAETHG_1371 | 0 | 11.03476 | 11.03476 |
| 7 | CAETHG_2006 | 0 | 10.68385 | 10.68385 |
| 8 | CAETHG_2909 | 1.515088 | 8.61957 | 10.13466 |
| 9 | CAETHG_2721 | 0 | 9.82919 | 9.82919 |
| 10 | CAETHG_2753 | 0.50272 | 9.082944 | 9.585664 |
| 11 | CAETHG_0160 | 0 | 8.767024 | 8.767024 |
| 12 | CAETHG_3299 | 0 | 7.876761 | 7.876761 |
| 13 | CAETHG_2751 | 4.149778 | 3.457399 | 7.607177 |
| 14 | CAETHG_0233 | 0 | 7.516631 | 7.516631 |
| 15 | CAETHG_1270 | 0 | 7.501854 | 7.501854 |
| 16 | CAETHG_0234 | 0 | 7.213378 | 7.213378 |
| 17 | CAETHG_2790 | 0 | 6.668087 | 6.668087 |
| 18 | CAETHG_2791 | 0 | 6.554927 | 6.554927 |
| 19 | CAETHG_2796 | 0 | 6.324132 | 6.324132 |

### Example 21

This example describes gene disruption targets to increase target compound production during autotrophic growth. This strategy involves eliminating or decreasing the production of other fermentation byproducts, making the target compound a required growth byproduct. Metabolic modeling experiments were performed as described in Example 1.

Modeling evidence demonstrates that this strategy is appropriate for target compounds whose production imposes minimal ATP burden. This strategy is not well suited for products with significant ATP burden during autotrophic growth. This is because this strategy decreases cellular ATP yields through the elimination of substrate level phosphorylation catalysed by acetate kinase.

In particular, production of products such as acetone, isopropanol, 1,3-butanediol, 3-hydroxybutyrate, 2-hydroxyisobutyrate, 3-hydroxyisovalerate, and adipic acid can be improved by introducing a disruptive mutation into genes encoding acetate kinase and/or phosphate transacetylase, and optionally further introducing a disruptive mutation into one or more genes encoding acetolactate decarboxylase, lactate dehydrogenase, aldehyde dehydrogenase, or citramalate synthase.

Each model was assessed using flux variability analysis to determine the minimum required flux towards the target compound during normal growth. Then, the proposed set of disruptive gene mutations was applied to each model. Flux variability analysis was carried out again to identify any existence of coupling between compound production and growth. Simulations were carried out using cobrapy version 0.13.4.

| | Minimum yield | Minimum yield during |
|---|---|---|
| | during cell | cell growth with target |
| Product | growth | genes disrupted |
| Acetone | 0.00 | 0.301 |
| 1,3-Butanediol | 0.00 | 0.323 |
| 3-Hydroxybutyrate | 0.00 | 0.395 |
| 2-Hydroxyisobutyrate | 0.00 | 0.395 |
| 3-Hydroxyisovalerate | 0.00 | 0.368 |
| Adipic acid | 0.00 | 0.428 |

### Example 22

This example describes increasing target compound production during autotrophic growth on gas mixes with a low proportion of CO by decreasing required acetate co-production. Metabolic modeling experiments were performed as described in Example 1.

The strategy involves adjusting the redox cofactor balance so there is excess NADPH. To maintain redox homeostasis, the cell must make products whose production pathway requires NADPH. As acetate production does not fulfil this, the cell will be required to make other products to achieve maximum growth rates.

Modeling evidence demonstrates that this strategy is appropriate for target compounds with an ATP burden that requires the co-production of acetate. This strategy is also appropriate for strains that produce ethanol as a primary product. This strategy is predicted to work on low CO gases, where the cell can utilise the hydrogenase enzyme to reduce ferredoxin and NAD(P)+. In some cases, the maximum possible yield of the target compound will decrease, as this strategy reduces the efficiency of the energy metabolism of the cell.

In particular, production of products such as ethanol, acetone, isopropanol, 1,3-butanediol, 2-butanol, 2-hydroxyisobutyrate, 3-hydroxyisovalerate, adipic acid, methyl ethyl ketone, isoprene, salicylate, chorismate, and farnesene can be improved by introducing a disruptive mutation into a gene encoding NAD-dependent electron-bifurcating [FeFe]-hydrogenase (e.g., Hyd), and optionally further introducing a disruptive mutation into one or more genes encoding glutamate synthase, citramalate synthase, acetolactate decarboxylase, or lactate dehydrogenase.

Each model was assessed using flux variability analysis to determine the minimum required flux to acetate at high growth rates. Then, the proposed set of disruptive gene mutations was applied to each model. The NAD-dependent hydrogenase (Hyd) was removed from the stoichiometric matrix to represent the knock out of this enzyme. Flux through the glutamate synthase reaction was decreased by 30% to represent a disruption of this enzyme. Flux variability analysis was carried out again to determine the minimum acetate production requirement to achieve maximum growth. Simulations were carried out using cobrapy version 0.13.4

Minimum required acetate yield at highest growth
rate (C-mol product/mol CO + H₂ uptake)

| | | Disrupted NAD- | Disrupted NAD-dependent electron- |
|---|---|---|---|
| | | dependent electron- | bifurcating [FeFe]-hydrogenase |
| | Parental | bifurcating [FeFe]- | (Hyd) and disrupted glutamate |
| Product | strain | hydrogenase (Hyd) | synthase (under expression) |
| Ethanol | 0.430 | 0.356 | 0.0002 |
| Acetone | 0.430 | 0.356 | 0.0002 |
| Isopropanol | 0.430 | 0.356 | 0.0002 |
| 1,3-Butanediol | 0.430 | 0.356 | 0.0000 |
| 2-Butanol | 0.430 | 0.356 | 0.0002 |
| 2-Hydroxyisobutyrate | 0.430 | 0.356 | 0.0000 |
| 3-Hydroxyisovalerate | 0.430 | 0.356 | 0.0000 |
| Adipic acid | 0.430 | 0.356 | 0.0002 |
| Methyl ethyl ketone | 0.430 | 0.356 | 0.0002 |
| Isoprene | 0.430 | 0.355 | 0.0000 |
| Salicylate | 0.430 | 0.356 | 0.0002 |
| Chorismate | 0.430 | 0.356 | 0.0002 |
| Farnesene | 0.430 | 0.356 | 0.0002 |

### Example 23

This example describes increasing flux through acetoacetyl-CoA, a central metabolic node. Increasing flux through this node will increase production of downstream products such as acetone, isopropanol, 3-hydroxyisovaleryl-CoA, 3-hydroxyisovalerate, isobutylene, isopentenyl pyrophosphate (IPP), dimethylallyl pyrophosphate (DMAPP), isoprene, terpenoids such as farnesene, 3-hydroxybutyryl-CoA, crotonyl-CoA, 3-hydroxybutyrate, 3-hydroxybutyrylaldehyde, 1,3-butanediol, 2-hydroxyisobutyryl-CoA, 2-hydroxyisobutyrate, butyryl-CoA, butyrate, butanol, caproate, hexanol, octanoate, octanol, 1,3-hexanediol, 2-buten-1-ol, isovaleryl-CoA, isovalerate, or isoamyl alcohol. Metabolic modeling experiments were performed as described in Example 1.

Most Wood-Ljungdahl microorganisms are not natively capable of converting acetyl-CoA to acetoacetyl-CoA, such that this step may require the introduction of a heterologous enzyme, such as a thiolase (i.e., acetyl-CoA acetyltransferase) (EC 2.3.1.9). The thiolase may be, for example, ThlA from Clostridium acetobutylicum (WP_010966157.1), PhaA from Cupriavidus necator (WP_013956452.1), BktB from Cupriavidus necator (WP_011615089.1), AtoB from Escherichia coli (NP_416728.1), or a similar.

In particular, flux through acetoacetyl-CoA can be improved by introducing a disruptive mutation into one or more genes encoding one or more, two or more, three or more, four or more, or five or more of NAD-dependent electron-bifurcating [FeFe]-hydrogenase (e.g., Hyd), glutamate synthase, citramalate synthase, acetolactate decarboxylase, lactate dehydrogenase, acetate kinase, phosphate transacetylase, or aldehyde dehydrogenase.

| | Reference number |
|---|---|
| Enzyme name | in C. autothanogenum |
| NAD-dependent | CAETHG_1576, CAETHG_1578, |
| electron-bifurcating | CAETHG_3569, CAETHG_3570, |
| [FeFe]-hydrogenase | CAETHG_3571 |
| Glutamate synthase | CAETHG_0477, CAETHG_1580, |
| | CAETHG_3850, CAETHG_3851 |
| Citramalate synthase | CAETHG_2751 |
| Acetolactate decarboxylase | CAETHG_2932 |
| Lactate dehydrogenase | CAETHG_1147 |
| Acetate kinase | CAETHG_3359 |
| Phosphate transacetylase | CAETHG_3358 |
| Aldehyde dehydrogenase | CAETHG_1819, CAETHG_3287, |
| | CAETHG_1830 |

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A non-naturally occurring Wood-Ljungdahl bacterium comprising a heterologous thiolase and a disruptive mutation in one or more genes encoding one or more of NAD-dependent electron-bifurcating [FeFe]-hydrogenase, glutamate synthase, citramalate synthase, acetolactate decarboxylase, lactate dehydrogenase, acetate kinase, phosphate transacetylase, and aldehyde dehydrogenase, wherein the non-naturally occurring bacterium has improved carbon flux through acetoacetyl-CoA compared to a parental bacterium.

2. The non-naturally occurring bacterium of claim 1, wherein expression of the one or more genes is decreased or eliminated compared to the parental bacterium.

3. The non-naturally occurring bacterium of claim 1, wherein the non-naturally occurring bacterium produces a product selected from the group consisting of acetone, isopropanol, 3-hydroxyisovaleryl-CoA, 3-hydroxyisovalerate, isobutylene, isopentenyl pyrophosphate, dimethylallyl pyrophosphate, isoprene, famesene, 3-hydroxybutyryl-CoA, crotonyl-CoA, 3-hydroxybutyrate, 3-hydroxybutyrylaldehyde, 1,3-butanediol, 2-hydroxyisobutyryl-CoA, 2-hydroxyisobutyrate, butyryl-CoA, butyrate, butanol, caproate, hexanol, octanoate, octanol, 1,3-hexanediol, 2-buten-1-ol, isovaleryl-CoA, isovalerate, or isoamyl alcohol.

4. The non-naturally occurring bacterium of claim 1, wherein the parental bacterium is selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi.

5. The non-naturally occurring bacterium of claim 1, wherein the parental bacterium is selected from the group consisting of Clostridium autoethanogenum, Clostridium ljungdahlii, and Clostridium ragsdalei.

6. The non-naturally occurring bacterium of claim 1, wherein the parental bacterium is Clostridium autoethanogenum and:
(a) the NAD-dependent electron-bifurcating [FeFe]-hydrogenase is selected from the group consisting of CAETHG_1576, CAETHG_1578, CAETHG_3569, CAETHG_3570, and CAETHG_3571,
(b) the glutamate synthase is selected from the group consisting of CAETHG_0477, CAETHG_1580, CAETHG_3850, and CAETHG_3851,
(c) the citramalate synthase is CAETHG_2751,
(d) the acetolactate decarboxylase is CAETHG_2932,
(e) the lactate dehydrogenase is CAETHG_1147,
(f) the acetate kinase is CAETHG_3359,
(g) the phosphate transacetylase is CAETHG_3358, or
(h) the aldehyde dehydrogenase is selected from the group consisting of CAETHG_1819, CAETHG_3287, and CAETHG_1830.

7. A method of producing a product by culturing the non-naturally occurring bacterium of claim 1 in the presence of a gaseous substrate comprising one or more of CO, CO₂, and H₂.

8. The method of claim 7, wherein the product is selected from the group consisting of acetone, isopropanol, 3-hydroxyisovaleryl-CoA, 3-hydroxyisovalerate, isobutylene, isopentenyl pyrophosphate, dimethylallyl pyrophosphate, isoprene, farnesene, 3-hydroxybutyryl-CoA, crotonyl-CoA, 3-hydroxybutyrate, 3-hydroxybutyrylaldehyde, 1,3-butanediol, 2-hydroxyisobutyryl-CoA, 2-hydroxyisobutyrate, butyryl-CoA, butyrate, butanol, caproate, hexanol, octanoate, octanol, 1,3-hexanediol, 2-buten-1-ol, isovaleryl-CoA, isovalerate, or isoamyl alcohol.

9. A non-naturally occurring Wood-Ljungdahl bacterium comprising a disruptive mutation in one or more genes, wherein the non-naturally occurring bacterium has improved carbon flux through chorismate compared to a parental bacterium.

10. The non-naturally occurring bacterium of claim 9, wherein the one or more genes encode one or more of purine-nucleoside phosphorylase, lactate permease, cystathionine gamma-lyase, adenine phosphoribosyltransferase, 5′-nucleotidase/3′-nucleotidase/exopolyphosphatase, small conductance mechanosensitive channel, arginine deiminase, LL-diaminopimelate aminotransferase apoenzyme, and phosphopentomutase.

11. The non-naturally occurring bacterium of claim 9, wherein expression of the one or more genes is decreased or eliminated compared to the parental bacterium.

12. The non-naturally occurring bacterium of claim 9, wherein the non-naturally occurring bacterium produces a product selected from the group consisting of chorismate, para-hydroxybenzoic acid, salicylate, 2-aminobenzoate, dihydroxybenzoate, 4-hydroxycyclohexane carboxylic acid, and salts and ions thereof.

13. The non-naturally occurring bacterium of claim 9, wherein the parental bacterium is selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi.

14. The non-naturally occurring bacterium of claim 9, wherein the parental bacterium is selected from the group consisting of Clostridium autoethanogenum, Clostridium ljungdahlii, and Clostridium ragsdalei.

15. The non-naturally occurring bacterium of claim 9, wherein:
(a) the parental bacterium is Clostridium autoethanogenum and the one or more genes encode one or more of CAETHG_0160, CAETHG_0248, CAETHG_0498, CAETHG_1270, CAETHG_1371, CAETHG_2107, CAETHG_3021, CAETHG_3510, and CAETHG_3924,
(b) the parental bacterium is Clostridium ljungdahlii and the one or more genes encode one or more of CLJU_c20750, CLJU_c21610, CLJU_c24380, CLJU_c33720, CLJU_c34740, CLJU_c42810, CLJU_c09270, CLJU_c14280, and CLJU_c18150, or
(c) the parental bacterium is Clostridium ragsdalei and the one or more genes encode one or more of CLRAG_19250, CLRAG_31200, CLRAG_25120, CLRAG_24560, CLRAG_14800, CLRAG_25620, CLRAG_09600, or CLRAG_00520.

16. A method of producing a product by culturing the non-naturally occurring bacterium of claim 9 in the presence of a gaseous substrate comprising one or more of CO, CO₂, and H₂.

17. The method of claim 16, wherein the non-naturally occurring bacterium produces a product selected from the group consisting of chorismate, para-hydroxybenzoic acid, salicylate, 2-aminobenzoate, dihydroxybenzoate, 4-hydroxycyclohexane carboxylic acid, and salts and ions thereof.
